(19) United States
(12) Patent Application Publication
(10) Pub. No.: US 2009/0222957 A1
(43) Pub. Date: Sep. 3, 2009

(54) REGULATORY PROTEIN-REGULATORY REGION ASSOCIATIONS RELATED TO ALKALOID BIOSYNTHESIS

(75) Inventors: Nestor Apuya, Culver City, CA (US); Joon-Hyun Park, Oak Park, CA (US); Steven Craig Bobzin, Malibu, CA (US)

Correspondence Address: FISH & RICHARDSON P.C., P.O. BOX 1022, MINNEAPOLIS, MN, 55440-1022, US

(73) Assignee: Ceres Inc., Thousand Oaks, CA (US)

(21) Appl. No.: 12/296,390

(22) PCT Filed: Apr. 6, 2007
(86) PCT No.: PCT/US2007/008859

§ 371 Date: May 11, 2009

Related U.S. Application Data
(60) Provisional application No. 60/790,489, filed on Apr. 7, 2006.

(51) Int. Cl. A01H 5/00; C12Q 1/00; C12N 5/10

(52) U.S. Cl. 800/298; 435/4; 435/419

(57) ABSTRACT

Materials and methods for identifying regulatory region-regulatory protein associations are disclosed. Materials and methods for modulating expression of a sequence of interest are disclosed.

### TECHNICAL FIELD

This document relates to materials and methods involved in modulating gene expression in plants. For example, this document relates to materials and methods for modulating the expression of nucleic acid sequences of interest, including both endogenous and exogenous nucleic acid sequences, such as those involved in alkaloid biosynthesis.

### INCORPORATION-BY-REFERENCE & TEXTS

The material on the accompanying diskette is hereby incorporated by reference into this application. The accompanying compact discs are identical and contain one file, 11696-140WO2-sequence.txt, which was created on Apr. 6, 2007. The file named 11696-140WO2-sequence.txt is 3,634 KB. The file can be accessed using Microsoft Word on a computer that uses Windows OS.

### BACKGROUND

Plant families that produce alkaloids include the Papaveraceae, Berberidaceae, Leguminosae, Boraginaceae, Apocynaceae, Asclepiadaceae, Liliaceae, Gnetaceae, Erythroxylaceae, Convolvulaceae, Ranunculaeceae, Rubiaceae, Solanaceae, and Rutaceae families. Many alkaloids isolated from such plants are known for their pharmacologic (e.g., narcotic), insecticidal, and physiologic effects. For example, the poppy (Papaveraceae) family contains about 250 species found mainly in the northern temperate regions of the world. The principal morphinan alkaloids in opium poppy (Papaver somniferum) are morphine, codeine, and thebaine, which are used directly or modified using synthetic methods to produce pharmaceutical compounds used for pain management, cough suppression, and addiction.

### SUMMARY

The present invention relates to materials and methods for modulating expression of nucleic acid sequences, such as those encoding polypeptides involved in biosynthesis of alkaloids. For example, the invention relates to the identification of regulatory proteins that are associated with regulatory regions, i.e., regulatory proteins that are capable of interacting either directly or indirectly with regulatory regions of genes encoding enzymes in an alkaloid biosynthesis pathway, and thereby modulating expression, e.g., transcription, of such genes. Modulation of expression can include up-regulation or activation, e.g., an increase of expression relative to basal or native states (e.g., a control level). In other cases, modulation of expression can include down-regulation or repression, e.g., a decrease of expression relative to basal or native states, such as the level in a control. In many cases, a regulatory protein is a transcription factor and its associated regulatory region is a promoter. Regulatory proteins identified as being capable of interacting directly or indirectly with regulatory regions of genes encoding enzymes in an alkaloid biosynthesis pathway can be used to create transgenic plants, e.g., plants capable of producing one or more alkaloids. Such plants can have modulated, e.g., increased, amounts and/or rates of biosynthesis of one or more alkaloid compounds. Regulatory proteins can also be used along with their cognate promoters to modulate transcription of one or more endogenous sequences, e.g., alkaloid biosynthesis genes, in a plant cell. Given the variety of uses of the various alkaloid classes of compounds, it would be useful to control selective expression of one or more proteins, including enzymes, regulatory proteins, and other auxiliary proteins, involved in alkaloid biosynthesis, e.g., to regulate biosynthesis of known and/or novel alkaloids.

In one aspect, a method of determining whether or not a regulatory region is activated by a regulatory protein is provided. The method comprises, or consists essentially of, determining whether or not reporter activity is detected in a plant cell transformed with (a) a recombinant nucleic acid construct comprising a regulatory region operably linked to a nucleic acid encoding a polypeptide having the reporter activity; and (b) a recombinant nucleic acid construct comprising a nucleic acid encoding a regulatory protein comprising a polypeptide sequence having 80% or greater sequence identity to a polypeptide sequence selected from the group consisting of SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NO:200, SEQ ID NO:205, SEQ ID NOs:211-214, SEQ ID NOs:216-223, SEQ ID NOs:225-226, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NO:632, SEQ ID NO:637, SEQ ID NO:639, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NO:703, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-907, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:1119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NO:1212, SEQ ID NOs:1220-1224, SEQ ID NOs:1226-1241, SEQ ID NOs:1243-1246, SEQ ID NO:1248, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NO:1394, SEQ ID NO:1401, SEQ ID NOs:1404-1411, SEQ ID NOs:1413-1414, SEQ ID NO:1421, SEQ ID NOs:1423-1427, SEQ ID NOs:1429-1438, SEQ ID NO:1440, SEQ ID NO:1452, SEQ ID NOs:1476-1484, and the consensus sequences set forth in FIGS. 1-140, where detection of the reporter activity indicates that the regulatory region is activated by the regulatory protein.

The activation can be direct or indirect. The nucleic acid encoding the regulatory protein can be operably linked to a regulatory region, where the regulatory region is capable of modulating expression of the regulatory protein. The regulatory region capable of modulating expression of the regulatory protein can be a promoter. The promoter can be a tissue-preferential promoter, such as a vascular tissue-preferential promoter or a poppy capsule-preferential promoter. The promoter can be an inducible promoter. The promoter can be a cell type-preferential promoter. The cell can be from a stem, seed pod, reproductive, or parenchymal tissue. The cell can be a laticifer, sieve element, or companion cell.

The plant cell can be stably transformed with the recombinant nucleic acid construct comprising a regulatory region operably linked to a nucleic acid encoding a polypeptide having a reporter activity and transiently transformed with the recombinant nucleic acid construct comprising the nucleic acid encoding the regulatory protein. The plant cell can be stably transformed with the recombinant nucleic acid construct comprising the nucleic acid encoding the regulatory protein and transiently transformed with the recombinant nucleic acid construct comprising the regulatory region operably linked to a nucleic acid encoding a polypeptide having a reporter activity. The plant cell can be stably transformed with the recombinant nucleic acid construct comprising the nucleic acid encoding the regulatory protein and stably transformed with the recombinant nucleic acid construct comprising the regulatory region operably linked to a nucleic acid encoding a polypeptide having a reporter activity. The plant cell can be transiently transformed with the recombinant nucleic acid construct comprising the nucleic acid encoding the regulatory protein and transiently transformed with the recombinant nucleic acid construct comprising the regulatory region operably linked to a nucleic acid encoding a polypeptide having a reporter activity.

The reporter activity can be selected from an enzymatic activity and an optical activity. The enzymatic activity can be selected from luciferase activity, neomycin phosphotransferase activity, and phosphinothricin acetyl transferase activity. The optical activity can be bioluminescence, fluorescence, or phosphorescence.

In another aspect, a method of determining whether or not a regulatory region is activated by a regulatory protein is provided. The method comprises determining whether or not reporter activity is detected in a plant cell transformed with (a) a recombinant nucleic acid construct comprising a regulatory region comprising a nucleic acid having 80% or greater sequence identity to a regulatory region selected from the group consisting of SEQ ID NOs:1453-1468 operably linked to a nucleic acid encoding a polypeptide having said reporter activity; and (b) a recombinant nucleic acid construct comprising a nucleic acid encoding a regulatory protein, where detection of the reporter activity indicates that the regulatory region is activated by the regulatory protein.

The regulatory protein can comprise a polypeptide sequence having 80% or greater sequence identity to a polypeptide sequence selected from the group consisting of SEQ ID NOs:80-84, SEQ ID NOs:86-91, SEQ ID NO:93, SEQ ID NOs:95-111, SEQ D NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NO:200, SEQ ID NO:205, SEQ ID NOs:211-214, SEQ ID NOs:216-223, SEQ ID NOs:225-226, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NO:632, SEQ ID NO:637, SEQ ID NO:639, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NO:703, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-907, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:1119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NO:1212, SEQ ID NOs:1220-1224, SEQ ID NOs:1226-1241, SEQ ID NOs:1243-1246, SEQ ID NO:1248, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NO:1394, SEQ ID NO:1401, SEQ ID NOs:1404-1411, SEQ ID NOs:1413-1414, SEQ ID NO:1421, SEQ ID NOs:1423-1427, SEQ ID NOs:1429-1438, SEQ ID NO:1440, SEQ ID NO:1452, SEQ ID NOs:1476-1484, and the consensus sequences set forth in FIGS. 1-140.

In another aspect, a plant cell is provided. The plant cell comprises an exogenous nucleic acid comprising a nucleic acid encoding a regulatory protein comprising a polypeptide sequence having 80% or greater sequence identity to a polypeptide sequence selected from the group consisting of SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NO:200, SEQ ID NO:205, SEQ ID NOs:211-214, SEQ ID NOs:216-223, SEQ ID NOs:225-226, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NO:632, SEQ ID NO:637, SEQ ID NO:639, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NO:703, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-907, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:1119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NO:1212, SEQ ID NOs:1220-1224, SEQ ID NOs:1226-1241, SEQ ID NOs:1243-1246, SEQ ID NO:1248, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NO:1394, SEQ ID NO:1401, SEQ ID NOs:1404-1411, SEQ ID NOs:1413-1414, SEQ ID NO:1421, SEQ ID NOs:1423-1427, SEQ ID NOs:1429-1438, SEQ ID NO:1440, SEQ ID NO:1452, SEQ ID NOs:1476-1484, and the consensus sequences set forth in FIGS. 1-140, wherein the nucleic acid is operably linked to a regulatory region that modulates transcription of the regulatory protein in the plant cell.

The regulatory region can be a promoter. The promoter can be a tissue-preferential promoter. The tissue can be vascular tissue or poppy capsule tissue. The tissue can be stem, seed pod, or parenchymal tissue. The tissue can be a reproductive tissue. The promoter can be a cell type-preferential promoter. The cell can be a laticifer cell, a companion cell, or a sieve element cell. The promoter can be an inducible promoter.

The plant cell can be capable of producing one or more alkaloids. The plant cell can further comprise an endogenous regulatory region that is associated with the regulatory protein. The regulatory protein can modulate transcription of an endogenous gene involved in alkaloid biosynthesis in the cell. The endogenous gene can comprise a coding sequence for an alkaloid biosynthesis enzyme. The endogenous gene can comprise a coding sequence for a regulatory protein involved in alkaloid biosynthesis. The modulation can be an increase in transcription of said endogenous gene.

The endogenous gene can be a tetrahydrobenzylisoquinoline alkaloid biosynthesis enzyme, a benzophenanthridine alkaloid biosynthesis enzyme, a morphinan alkaloid biosynthesis enzyme, a monoterpenoid indole alkaloid biosynthesis enzyme, a bisbenzylisoquinoline alkaloid biosynthesis enzyme, a pyridine, purine, tropane, or quinoline alkaloid biosynthesis enzyme, a terpenoid, betaine, or phenethylamine alkaloid biosynthesis enzyme, or a steroid alkaloid biosynthesis enzyme.

The endogenous gene can be selected from the group consisting of tyrosine decarboxylase (YDC or TYD; EC 4.1.1.25), norcoclaurine synthase (EC 4.2.1.78), coclaurine N-methyltransferase (EC 2.1.1.140), (R,S)-norcoclaurine 6-O-methyl transferase (NOMT; EC 2.1.1.128), S-adenosyl-L-methionine:3′-hydroxy-N-methylcoclaurine 4′-O-methyltransferase 1 (HMCOMT 1; EC 2.1.1.116); S-adenosyl-L-methionine:3′-hydroxy-N-methylcoclaurine 4′-O-methyltransferase 2 (HMCOMT2; EC 2.1.1.116); monophenol monooxygenase (EC1.14.18.1), N-methylcoclaurine 3′-hydroxylase (NMCH; EC 1.14.13.71), (R,S)-reticuline 7-O-methyltransferase (ROMT); berbamunine synthase (EC 1.14.21.3), columbamine O-methyltransferase (EC 2.1.1.118), berberine bridge enzyme (BBE; (EC 1.21.3.3), reticuline oxidase (EC 1.21.3.4), dehydro reticulinium ion reductase (EC 1.5.1.27), (RS)-1-benzyl-1,2,3,4-tetrahydroisoquinoline N-methyltransferase (EC 2.1.1.115), (S)-scoulerine oxidase (EC 1.14.21.2), (S)-cheilanthifoline oxidase (EC 1.14.21.1), (S)-tetrahydroprotoberberine N-methyltransferase (EC 2.1.1.122), (S)-canadine synthase (EC 1.14.21.5), tetrahydroberberine oxidase (EC 1.3.3.8), and columbamine oxidase (EC 1.21.3.2).

The endogenous gene can be selected from the group consisting of those encoding for dihydrobenzophenanthridine oxidase (EC 1.5.3.12), dihydrosanguinarine 10-hydroxylase (EC 1.14.13.56), 10-hydroxydihydrosanguinarine 10-O-methyltransferase (EC 2.1.1.119), dihydrochelirubine 12-hydroxylase (EC 1.14.13.57), and 12-hydroxydihydrochelirubine 12-O-methyltransferase (EC 2.1.1.120).

The endogenous gene can be selected from the group consisting of those encoding for salutaridinol 7-O-acetyltransferase (SAT; EC 2.3.1.150), salutaridine synthase (EC 1.14.21.4), salutaridine reductase (EC 1.1.1.248), morphine 6-dehydrogenase (EC 1.1.1.218); and codeinone reductase (CR; EC 1.1.1.247).

The plant cell can further comprise an exogenous regulatory region operably linked to a sequence of interest, where the exogenous regulatory region is associated with the regulatory protein, and where the exogenous regulatory region comprises a nucleic acid having 80% or greater sequence identity to a regulatory region selected from the group consisting of SEQ ID NOs:1453-1468.

A plant cell described above can be capable of producing one or more alkaloids. An alkaloid can be a morphinan alkaloid, a morphinan analog alkaloid, a tetrahydrobenzylisoquinoline alkaloid, a benzophenanthridine alkaloid, a monoterpenoid indole alkaloid, a bisbenzylisoquinoline alkaloid, a pyridine, purine, tropane, or quinoline alkaloid, a terpenoid, betaine, or phenethylamine alkaloid, or a steroid alkaloid.

A plant cell described above can be a member of the Papaveraceae, Menispermaceae, Lauraceae, Euphorbiaceae, Berberidaceae, Leguminosae, Boraginaceae, Apocynaceae, Asclepiadaceae, Liliaceae, Gnetaceae, Erythroxylaceae, Convolvulaceae, Ranunculaeceae, Rubiaceae, Solanaceae, or Rutaceae families. A plant cell described above can be a member of the species Papaver bracteatum, Papaver orientale, Papaver setigerum, Papaver somniferum, Croton salutaris, Croton balsamifera, Sinomenium acutum, Stephania cepharantha, Stephania zippeliana, Litsea sebiferea, Alseodaphne perakensis, Cocculus laurifolius, Duguetia obovata, Rhizocarya racemifera, or Beilschmiedia oreophila.

A plant cell described above can further comprise a nucleic acid encoding a second regulatory protein operably linked to a second regulatory region that modulates transcription of the second regulatory protein in the plant cell. The nucleic acid encoding a second regulatory protein operably linked to a second regulatory region can be present on a second recombinant nucleic acid construct.

The sequence of interest can comprise a coding sequence for a polypeptide involved in alkaloid biosynthesis. The polypeptide can be a regulatory protein involved in alkaloid biosynthesis. The polypeptide can be an alkaloid biosynthesis enzyme. The enzyme can be a morphinan alkaloid biosynthesis enzyme, a tetrahydrobenzylisoquinoline alkaloid biosynthesis enzyme, a benzophenanthridine alkaloid biosynthesis enzyme, a monoterpenoid indole alkaloid biosynthesis enzyme, a bisbenzylisoquinoline alkaloid biosynthesis enzyme, a pyridine, purine, tropane, or quinoline alkaloid biosynthesis enzyme, a terpenoid, betaine, or phenethylamine alkaloid biosynthesis enzyme, or a steroid alkaloid biosynthesis enzyme.

The enzyme can be selected from the group consisting of salutaridinol 7-O-acetyltransferase (SAT; EC 2.3.1.150), salutaridine synthase (EC 1.14.21.4), salutaridine reductase (EC 1.1.1.248), morphine 6-dehydrogenase (EC 1.1.1.218); and codeinone reductase (CR; EC 1.1.1.247).

The enzyme can be selected from the group consisting of tyrosine decarboxylase (YDC or TYD; EC 4.1.1.25), norcoclaurine synthase (EC 4.2.1.78), coclaurine N-methyltransferase (EC 2.1.1.140), (R,S)-norcoclaurine 6-O-methyl transferase (NOMT; EC 2.1.1.128), S-adenosyl-L-methionine:3′-hydroxy-N-methylcoclaurine 4′-O-methyltransferase 1 (HMCOMT1; EC 2.1.1.116); S-adenosyl-L-methionine:3′-hydroxy-N-methylcoclaurine 4′-O-methyltransferase 2 (HMCOMT2; EC 2.1.1.116); monophenol monooxygenase (EC 1.14.18.1), N-methylcoclaurine 3′-hydroxylase (NMCH; EC 1.14.13.71), (R,S)-reticuline 7-O-methyltransferase (ROMT); berbamunine synthase (EC 1.14.21.3), columbamine O-methyltransferase (EC 2.1.1.118), berberine bridge enzyme (BBE; (EC 1.21.3.3), reticuline oxidase (EC 1.21.3.4), dehydro reticulinium ion reductase (EC 1.5.1.27), (RS)-1-benzyl-1,2,3,4-tetrahydroisoquinoline N-methyltransferase (EC 2.1.1.115), (S)-scoulerine oxidase (EC 1.14.21.2), (S)-cheilanthifoline oxidase (EC 1.14.21.1), (S)-tetrahydroprotoberberine N-methyltransferase (EC 2.1.1.122), (S)-canadine synthase (EC 1.14.21.5), tetrahydroberberine oxidase (EC 1.3.3.8), and columbamine oxidase (EC 1.21.3.2).

The enzyme can be selected from the group consisting of dihydrobenzophenanthridine oxidase (EC 1.5.3.12), dihydrosanguinarine 10-hydroxylase (EC 1.14.13.56), 10-hydroxydihydrosanguinarine 10-O-methyltransferase (EC 2.1.1.119), dihydrochelirubine 12-hydroxylase (EC 1.14.13.57), and 12-hydroxydihydrochelirubine 12-O-methyltransferase (EC 2.1.1.120).

A regulatory protein-regulatory region association can be effective for modulating the amount of at least one alkaloid compound in the cell. An alkaloid compound can be selected from the group consisting of salutaridine, salutaridinol, salutaridinol acetate, thebaine, isothebaine, papaverine, narcotine, noscapine, narceine, hydrastine, oripavine, morphinone, morphine, codeine, codeinone, and neopinone. An alkaloid compound can be selected from the group consisting of berberine, palmatine, tetrahydropalmatine, S-canadine, columbamine, S-tetrahydrocolumbamine, S-scoulerine, S-cheilathifoline, S-stylopine, S-cis-N-methylstylopine, protopine, 6-hydroxyprotopine, R-norreticuline, S-norreticuline, R-reticuline, S-reticuline, 1,2-dehydroreticuline, S-3′-hydroxycoclaurine, S-norcoclaurine, S-coclaurine, S—N-methylcoclaurine, berbamunine, 2′-norberbamunine, and guatteguamerine. An alkaloid compound can be selected from the group consisting of dihydro-sanguinarine, sanguinarine, dihydroxy-dihydro-sanguinarine, 12-hydroxy-dihydrochelirubine, 10-hydroxy-dihydro-sanguinarine, dihydro-macarpine, dihydro-chelirubine, dihydro-sanguinarine, chelirubine, 12-hydroxy-chelirubine, and macarpine.

In another aspect, a Papaveraceae plant is provided. The plant comprises an exogenous nucleic acid comprising a nucleic acid encoding a regulatory protein comprising a polypeptide sequence having 80% or greater sequence identity to a polypeptide sequence selected from the group consisting of SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NO:200, SEQ ID NO:205, SEQ ID NOs:211-214, SEQ ID NOs:216-223, SEQ ID NOs:225-226, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NO:632, SEQ ID NO:637, SEQ ID NO:639, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NO:703, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-907, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:11119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NO:1212, SEQ ID NOs:1220-1224, SEQ ID NOs:1226-1241, SEQ ID NOs:1243-1246, SEQ ID NO:1248, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NO:1394, SEQ ID NO:1401, SEQ ID NOs:1404-1411, SEQ ID NOs:1413-1414, SEQ ID NO:1421, SEQ ID NOs:1423-1427, SEQ ID NOs:1429-1438, SEQ ID NO:1440, SEQ ID NO:1452, SEQ ID NOs:1476-1484, and the consensus sequences set forth in FIGS. 1-140, where the nucleic acid is operably linked to a regulatory region that modulates transcription of the regulatory protein in the plant cell.

In another aspect, a method of expressing a sequence of interest is provided. The method comprises, or consists essentially of, growing a plant cell comprising (a) an exogenous nucleic acid comprising a regulatory region comprising a nucleic acid having 80% or greater sequence identity to a regulatory region selected from the group consisting of SEQ ID NOs:1453-1468, where the regulatory region is operably linked to a sequence of interest; and (b) an exogenous nucleic acid comprising a nucleic acid encoding a regulatory protein comprising a polypeptide sequence having 80% or greater sequence identity to a polypeptide sequence selected from the group consisting of SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NO:200, SEQ ID NO:205, SEQ ID NOs:211-214, SEQ ID NOs:216-223, SEQ ID NOs:225-226, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NO:632, SEQ ID NO:637, SEQ ID NO:639, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NO:703, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-907, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:11119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NO:1212, SEQ ID NOs:1220-1224, SEQ ID NOs:1226-1241, SEQ ID NOs:1243-1246, SEQ ID NO:1248, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NO:1394, SEQ ID NO:1401, SEQ ID NOs:1404-1411, SEQ ID NOs:1413-1414, SEQ ID NO:1421, SEQ ID NOs:1423-1427, SEQ ID NOs:1429-1438, SEQ ID NO:1440, SEQ ID NO:1452, SEQ ID NOs:1476-1484, and the consensus sequences set forth in FIGS. 1-140; where the regulatory region and the regulatory protein are associated, and where the plant cell is grown under conditions effective for the expression of the regulatory protein.

In another aspect, a method of expressing an endogenous sequence of interest is provided. The method comprises, or consists essentially of, growing a plant cell comprising an endogenous regulatory region operably linked to a sequence of interest, where the endogenous regulatory region comprises a nucleic acid having 80% or greater sequence identity to a regulatory region selected from the group consisting of SEQ ID NOs:1453-1468, where the plant cell further comprises a nucleic acid encoding an exogenous regulatory protein, the exogenous regulatory protein comprising a polypeptide sequence having 80% or greater sequence identity to a polypeptide sequence selected from the group consisting of SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NO:200, SEQ ID NO:205, SEQ ID NOs:211-214, SEQ ID NOs:216-223, SEQ ID NOs:225-226, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NO:632, SEQ ID NO:637, SEQ ID NO:639, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NO:703, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-907, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:1119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NO:1212, SEQ ID NOs:1220-1224, SEQ ID NOs:1226-1241, SEQ ID NOs:1243-1246, SEQ ID NO:1248, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NO:1394, SEQ ID NO:1401, SEQ ID NOs:1404-1411, SEQ ID NOs:1413-1414, SEQ ID NO:1421, SEQ ID NOs:1423-1427, SEQ ID NOs:1429-1438, SEQ ID NO:1440, SEQ ID NO:1452, SEQ ID NOs:1476-1484, and the consensus sequences set forth in FIGS. 1-140, where the exogenous regulatory protein and the endogenous regulatory region are associated, and where the plant cell is grown under conditions effective for the expression of the exogenous regulatory protein.

In another aspect, a method of expressing an exogenous sequence of interest is provided. The method comprises, or consists essentially of, growing a plant cell comprising an exogenous regulatory region operably linked to a sequence of interest, where the exogenous regulatory region comprises a nucleic acid having 80% or greater sequence identity to a regulatory region selected from the group consisting of SEQ ID NOs:1453-1468, where the plant cell further comprises a nucleic acid encoding an endogenous regulatory protein, the endogenous regulatory protein comprising a polypeptide sequence having 80% or greater sequence identity to a polypeptide sequence selected from the group consisting of SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NO:200, SEQ ID NO:205, SEQ ID NOs:211-214, SEQ ID NOs:216-223, SEQ ID NOs:225-226, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NO:632, SEQ ID NO:637, SEQ ID NO:639, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NO:703, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-907, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:1119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NO:1212, SEQ ID NOs:1220-1224, SEQ ID NOs:1226-1241, SEQ ID NOs:1243-1246, SEQ ID NO:1248, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NO:1394, SEQ ID NO:1401, SEQ ID NOs:1404-1411, SEQ ID NOs:1413-1414, SEQ ID NO:1421, SEQ ID NOs:1423-1427, SEQ ID NOs:1429-1438, SEQ ID NO:1440, SEQ ID NO:1452, SEQ ID NOs:1476-1484, and the consensus sequences set forth in FIGS. 1-140, where the regulatory region and the regulatory protein are associated, and where the plant cell is grown under conditions effective for the expression of the endogenous regulatory protein.

The sequence of interest can comprise a coding sequence for a polypeptide involved in alkaloid biosynthesis. The nucleic acid encoding the exogenous regulatory protein can be operably linked to a regulatory region capable of modulating expression of the exogenous regulatory protein in the plant cell. The regulatory region capable of modulating expression of the exogenous regulatory protein in the plant cell can be selected from a tissue-specific, cell-specific, organ-specific, or inducible promoter. The regulatory region capable of modulating expression of the exogenous regulatory protein can be a vascular tissue-preferential promoter or a poppy capsule-preferential promoter.

In another aspect, a method of expressing a sequence of interest is provided. The method comprises, or consists essentially of, growing a plant cell comprising an exogenous nucleic acid. The exogenous nucleic acid comprises a nucleic acid encoding a regulatory protein comprising a polypeptide sequence having 80% or greater sequence identity to a polypeptide sequence selected from the group consisting of SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NO:200, SEQ ID NO:205, SEQ ID NOs:211-214, SEQ ID NOs:216-223, SEQ ID NOs:225-226, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NO:632, SEQ ID NO:637, SEQ ID NO:639, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NO:703, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-907, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:1119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NO:1212, SEQ ID NOs:1220-1224, SEQ ID NOs:1226-1241, SEQ ID NOs:1243-1246, SEQ ID NO:1248, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NO:1394, SEQ ID NO:1401, SEQ ID NOs:1404-1411, SEQ ID NOs:1413-1414, SEQ ID NO:1421, SEQ ID NOs:1423-1427, SEQ ID NOs:1429-1438, SEQ ID NO:1440, SEQ ID NO:1452, SEQ ID NOs:1476-1484, and the consensus sequences set forth in FIGS. 1-140. The nucleic acid is operably linked to a regulatory region that modulates transcription of the regulatory protein in the plant cell. The plant cell further comprises an exogenous regulatory region operably linked to a sequence of interest, where the exogenous regulatory region is associated with the regulatory protein, and where the exogenous regulatory region comprises a nucleic acid having 80% or greater sequence identity to a regulatory region selected from the group consisting of SEQ ID NOs:1453-1468. The plant cell is grown under conditions effective for the expression of the regulatory protein.

In another aspect, a method of modulating the expression level of one or more endogenous Papaveraceae genes involved in alkaloid biosynthesis is provided. The method comprises, or consists essentially of, transforming a cell of a member of the Papaveraceae family with a recombinant nucleic acid construct, where the nucleic acid construct comprises a nucleic acid encoding a regulatory protein comprising a polypeptide sequence selected from the group consisting of SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NO:200, SEQ ID NO:205, SEQ ID NOs:211-214, SEQ ID NOs:216-223, SEQ ID NOs:225-226, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NO:632, SEQ ID NO:637, SEQ ID NO:639, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NO:703, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-907, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:1119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NO:1212, SEQ ID NOs:1220-1224, SEQ ID NOs:1226-1241, SEQ ID NOs:1243-1246, SEQ ID NO:1248, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NO:1394, SEQ ID NO:1401, SEQ ID NOs:1404-1411, SEQ ID NOs:1413-1414, SEQ ID NO:1421, SEQ ID NOs:1423-1427, SEQ ID NOs:1429-1438, SEQ ID NO:1440, SEQ ID NO:1452, SEQ ID NOs:1476-1484, and the consensus sequences set forth in FIGS. 1-140, and where the nucleic acid is operably linked to a regulatory region that modulates transcription in the family member.

In another aspect, a method of producing one or more alkaloids in a plant cell is provided. The method comprises or consists essentially of, growing a plant cell comprising an exogenous nucleic acid. The exogenous nucleic acid comprises a nucleic acid encoding a regulatory protein comprising a polypeptide sequence having 80% or greater sequence identity to a polypeptide sequence selected from the group consisting of SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NO:200, SEQ ID NO:205, SEQ ID NOs:211-214, SEQ ID NOs:216-223, SEQ ID NOs:225-226, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NO:632, SEQ ID NO:637, SEQ ID NO:639, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NO:703, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-907, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ED NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:1119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NO:1212, SEQ ID NOs:1220-1224, SEQ ID NOs:1226-1241, SEQ ID NOs:1243-1246, SEQ ID NO:1248, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NO:1394, SEQ ID NO:1401, SEQ ID NOs:1404-1411, SEQ ID NOs:1413-1414, SEQ ID NO:1421, SEQ ID NOs:1423-1427, SEQ ID NOs:1429-1438, SEQ ID NO:1440, SEQ ID NO:1452, SEQ ID NOs:1476-1484, and the consensus sequences set forth in FIGS. 1-140. The nucleic acid is operably linked to a regulatory region that modulates transcription of the regulatory protein in the plant cell. The plant cell further comprises an endogenous regulatory region that is associated with the regulatory protein. The endogenous regulatory region is operably linked to a sequence of interest comprising a coding sequence for a polypeptide involved in alkaloid biosynthesis. The plant cell is capable of producing one or more alkaloids. The plant cell is grown under conditions effective for the expression of the regulatory protein.

In another aspect, a method of producing one or more alkaloids in a plant cell is provided. The method comprises, or consists essentially of, growing a plant cell comprising an exogenous nucleic acid. The exogenous nucleic acid comprises a nucleic acid encoding a regulatory protein comprising a polypeptide sequence having 80% or greater sequence identity to a polypeptide sequence selected from the group consisting of SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NO:200, SEQ ID NO:205, SEQ ID NOs:211-214, SEQ ID NOs:216-223, SEQ ID NOs:225-226, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NO:632, SEQ ID NO:637, SEQ ID NO:639, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NO:703, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-907, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:1119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NO:1212, SEQ ID NOs:1220-1224, SEQ ID NOs:1226-1241, SEQ ID NOs:1243-1246, SEQ ID NO:1248, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NO:1394, SEQ ID NO:1401, SEQ ID NOs:1404-1411, SEQ ID NOs:1413-1414, SEQ ID NO:1421, SEQ ID NOs:1423-1427, SEQ ID NOs:1429-1438, SEQ ID NO:1440, SEQ ID NO:1452, SEQ ID NOs:1476-1484, and the consensus sequences set forth in FIGS. 1-140. The nucleic acid is operably linked to a regulatory region that modulates transcription of the regulatory protein in the plant cell. The plant cell further comprises an exogenous regulatory region operably linked to a sequence of interest. The exogenous regulatory region is associated with the regulatory protein, and the exogenous regulatory region comprises a nucleic acid having 80% or greater sequence identity to a regulatory region selected from the group consisting of SEQ ID NOs:1453-1468. The sequence of interest comprises a coding sequence for a polypeptide involved in alkaloid biosynthesis. The plant cell is grown under conditions effective for the expression of the regulatory protein.

In another aspect, a method of modulating an amount of one or more alkaloid compounds in a Papaveraceae family member is provided. The method comprises, or consists essentially of, transforming a member of the Papaveraceae family with a recombinant nucleic acid construct. The nucleic acid construct comprises a nucleic acid encoding a regulatory protein comprising a polypeptide sequence selected from the group consisting of SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NO:200, SEQ ID NO:205, SEQ ID NOs:211-214, SEQ ID NOs:216-223, SEQ ID NOs:225-226, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NO:632, SEQ ID NO:637, SEQ ID NO:639, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NO:703, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-907, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:1119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NO:1212, SEQ ID NOs:1220-1224, SEQ ID NOs:1226-1241, SEQ ID NOs:1243-1246, SEQ ID NO:1248, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NO:1394, SEQ ID NO:1401, SEQ ID NOs:1404-1411, SEQ ID NOs:1413-1414, SEQ ID NO:1421, SEQ ID NOs:1423-1427, SEQ ID NOs:1429-1438, SEQ ID NO:1440, SEQ ID NO:1452, SEQ ID NOs:1476-1484, and the consensus sequences set forth in FIGS. 1-140. The nucleic acid is operably linked to a regulatory region that modulates transcription in the family member.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is an alignment of the amino acid sequence of Lead cDNA ID 23798983 (SEQ ID NO:80) with homologous and/or orthologous amino acid sequences CeresClone:916120 (SEQ ID NO:81), CeresClone:464614 (SEQ ID NO:82), and gi|62320596 (SEQ ID NO:83). The consensus sequence determined by the alignment is set forth.

FIG. 2 is an alignment of the amino acid sequence of Lead cDNA ID 23389356 (SEQ ID NO:86) with homologous and/or orthologous amino acid sequences CeresClone:1446017 (SEQ ID NO:87), gi|53370700 (SEQ ID NO:88), CeresClone:316709 (SEQ ID NO:89), and CeresClone:284127 (SEQ ID NO:91). The consensus sequence determined by the alignment is set forth.

FIG. 3 is an alignment of the amino acid sequence of Lead cDNA ID 23693590 (SEQ ID NO:95) with homologous and/or orthologous amino acid sequences gi|1370160 (SEQ ID NO:96), gi|560504 (SEQ ID NO:97), CeresClone:6827 (SEQ ID NO:99), gi|5714658 (SEQ ID NO:100), gi|34913324 (SEQ ID NO:102), CeresClone:221941 (SEQ ID NO:103), gi|303730 (SEQ ID NO:104), gi|218228 (SEQ ID NO:105), CeresClone:789317 (SEQ ID NO:106), CeresClone:1068093 (SEQ ID NO:107), gi|974778 (SEQ ID NO:109), gi|3025293 (SEQ ID NO:10), and gi|6688535 (SEQ ID NO:111). The consensus sequence determined by the alignment is set forth.

FIG. 4 is an alignment of the amino acid sequence of Lead cDNA ID 23663607 (SEQ ID NO:115) with homologous and/or orthologous amino acid sequences gi|34911396 (SEQ ID NO:116), gi|12324210 (SEQ ID NO:117), and gi|56784967 (SEQ ID NO:118). The consensus sequence determined by the alignment is set forth.

FIG. 5 is an alignment of the amino acid sequence of Lead cDNA ID 23522096 (5109D12; SEQ ID NO:123) with homologous and/or orthologous amino acid sequences gi|30523252 (SEQ ID NO:124), CeresClone:244495 (SEQ ID NO:125), gi|45181459 (SEQ ID NO:127), gi|52789958 (SEQ ID NO:128), gi|82313 (SEQ ID NO:129), gi|20219014 (SEQ ID NO:130), gi|6580941 (SEQ ID NO:131), gi|45268960 (SEQ ID NO:132), gi|55792842 (SEQ ID NO:133), gi|6580939 (SEQ ID NO:134), gi|46917358 (SEQ ID NO:135), gi|30523364 (SEQ ID NO:136), gi|55792848 (SEQ ID NO:137), gi|22091477 (SEQ ID NO:138), and gi|5031217 (SEQ ID NO:139). The consensus sequence determined by the alignment is set forth.

FIG. 6 is an alignment of the amino acid sequence of Lead cDNA ID 23447462 (5109E7; SEQ ID NO:141) with homologous and/or orthologous amino acid sequence gi|50923905 (SEQ ID NO:142). The consensus sequence determined by the alignment is set forth.

FIG. 7 is an alignment of the amino acid sequence of Lead cDNA ID 23499985 (5109F10; SEQ ID NO:144) with homologous and/or orthologous amino acid sequences gi|1076760 (SEQ ID NO:145), gi|1869928 (SEQ ID NO:147), CeresClone:986028 (SEQ ID NO:148), gi|12039274 (SEQ ID NO:149), and gi|463212 (SEQ ID NO:150). The consensus sequence determined by the alignment is set forth.

FIG. 8 is an alignment of the amino acid sequence of Lead cDNA ID 24374230 (5109G4; SEQ ID NO:158) with homologous and/or orthologous amino acid sequences CeresClone:1507510 (SEQ ID NO:159), CeresClone:602357 (SEQ ID NO:160), gi|50931081 (SEQ ID NO:163), CeresClone:500887 (SEQ ID NO:164), and CeresClone:702388 (SEQ ID NO:166). The consensus sequence determined by the alignment is set forth.

FIG. 9 is an alignment of the amino acid sequence of Lead cDNA ID 23547976 (5109G9; SEQ ID NO:168) with homologous and/or orthologous amino acid sequences CeresClone:1358913 (SEQ ID NO:169), gi|20340241 (SEQ ID NO:170), and gi|37901055 (SEQ ID NO:171). The consensus sequence determined by the alignment is set forth.

FIG. 10 is an alignment of the amino acid sequence of Lead cDNA ID 13653045 (5110A5; SEQ ID NO:173) with homologous and/or orthologous amino acid sequences gi|11385590_T (SEQ ID NO:180), gi|11385596_T (SEQ ID NO:181), gi|57899209_T (SEQ ID NO:182), CeresClone:1563222_T (SEQ ID NO:183), gi|11385602_T (SEQ ID NO:184), and gi|38564733_T (SEQ ID NO:185). The consensus sequence determined by the alignment is set forth.

FIG. 11 is an alignment of the amino acid sequence of Lead cDNA ID 23477523 (5110B9; SEQ ID NO:187) with homologous and/or orthologous amino acid sequences gi|9967526 (SEQ ID NO:188), gi|50511733 (SEQ ID NO:189), and gi|50511731 (SEQ ID NO:190). The consensus sequence determined by the alignment is set forth.

FIG. 12 is an alignment of the amino acid sequence of Lead cDNA ID 13610509 (5110E11; SEQ ID NO:200) with homologous and/or orthologous amino acid sequences CeresClone:514234 (SEQ ID NO:201), gi|66947626 (SEQ ID NO:202), and CeresClone:324706 (SEQ ID NO:203). The consensus sequence determined by the alignment is set forth.

FIG. 13 is an alignment of the amino acid sequence of Lead cDNA ID 23503364 (5110F5; SEQ ID NO:205) with homologous and/or orthologous amino acid sequences CeresClone:475115 (SEQ ID NO:206), CeresClone:925463 (SEQ ID NO:207), gi|34902824 (SEQ ID NO:208), and CeresClone:281953 (SEQ ID NO:209). The consensus sequence determined by the alignment is set forth.

FIG. 14 is an alignment of the amino acid sequence of Lead cDNA ID 12676498 (5110F8; SEQ ID NO:211) with homologous and/or orthologous amino acid sequences gi|34895192 (SEQ ID NO:212) and gi|2959360 (SEQ ID NO:213). The consensus sequence determined by the alignment is set forth.

FIG. 15 is an alignment of the amino acid sequence of Lead cDNA ID 4984839 (5110G8; SEQ ID NO:216) with homologous and/or orthologous amino acid sequences gi|31580813 (SEQ ID NO:217) and gi|30523252 (SEQ ID NO:223). The consensus sequence determined by the alignment is set forth.

FIG. 16 is an alignment of the amino acid sequence of Lead cDNA ID 23544026 (SEQ ID NO:225) with homologous and/or orthologous amino acid sequences CeresClone:2553 (SEQ ID NO:226) and CeresClone:659863 (SEQ ID NO:227). The consensus sequence determined by the alignment is set forth.

FIG. 17 is an alignment of the amino acid sequence of Lead cDNA ID 13579142 (5111E1; SEQ ID NO:229) with homologous and/or orthologous amino acid sequences CeresClone:463860 (SEQ ID NO:230), gi|50927857 (SEQ ID NO:231), CeresClone:296774 (SEQ ID NO:232), and CeresClone:843076 (SEQ ID NO:233). The consensus sequence determined by the alignment is set forth.

FIG. 18 is an alignment of the amino acid sequence of Lead cDNA ID 23365150 (SEQ ID NO:235) with homologous and/or orthologous amino acid sequences gi|4996642 (SEQ ID NO:236), gi|50253202 (SEQ ID NO:237), gi|47900733 (SEQ ID NO:238), gi|7489820 (SEQ ID NO:239), gi|4996644 (SEQ ID NO:240), gi|37051125 (SEQ ID NO:241), CeresClone:543840 (SEQ ID NO:242), gi|33332411 (SEQ ID NO:243), and gi|42556524 (SEQ ID NO:244). The consensus sequence determined by the alignment is set forth.

FIG. 19 is an alignment of the amino acid sequence of Lead cDNA ID 23411827 (SEQ ID NO:246) with homologous and/or orthologous amino acid sequences gi|20259679 (SEQ ID NO:247), gi|34900512 (SEQ ID NO:249), gi|51100730 (SEQ ID NO:250), gi|46395277 (SEQ ID NO:251), CeresClone:374770 (SEQ ID NO:252), gi|5081557 (SEQ ID NO:253), gi|53830033 (SEQ ID NO:254), gi|53801434 (SEQ ID NO:255), gi|53830021 (SEQ ID NO:256), gi|53830029 (SEQ ID NO:257), and gi|53830035 (SEQ ID NO:258). The consensus sequence determined by the alignment is set forth.

FIG. 20 is an alignment of the amino acid sequence of Lead cDNA ID 23370190 (SEQ ID NO:260) with homologous and/or orthologous amino acid sequences CeresClone:287298 (SEQ ID NO:261), CeresClone:533616 (SEQ ID NO:262), gi|38196013 (SEQ ID NO:1476), gi|60460512 (SEQ ID NO:1477), gi|38260661 (SEQ ID NO:1478), CeresClone:1242254 (SEQ ID NO:1479), gi|38260624 (SEQ ID NO:1480), gi|34906436 (SEQ ID NO:1481), gi|56605376 (SEQ ID NO:1482), CeresClone:673872 (SEQ ID NO:1483), and CeresClone:997341 (SEQ ID NO:1484). The consensus sequence determined by the alignment is set forth.

FIG. 21 is an alignment of the amino acid sequence of Lead cDNA ID 23367111 (SEQ ID NO:264) with homologous and/or orthologous amino acid sequences gi|55585713 (SEQ ID NO:265), gi|30526297 (SEQ ID NO:266), gi|57012875 (SEQ ID NO:267), gi|57012757 (SEQ ID NO:268), CeresClone:953351 (SEQ ID NO:269), gi|4099914 (SEQ ID NO:270), gi|50931913 (SEQ ID NO:271), gi|4099921 (SEQ ID NO:272), gi|37625035 (SEQ ID NO:273), CeresClone:326267 (SEQ ID NO:274), gi|28274832 (SEQ ID NO:275), gi|55824383 (SEQ ID NO:276), CeresClone:554848 (SEQ ID NO:277), gi|55419650 (SEQ ID NO:278), and CeresClone:280241 (SEQ ID NO:279). The consensus sequence determined by the alignment is set forth.

FIG. 22 is an alignment of the amino acid sequence of Lead cDNA ID 23364997 (SEQ ID NO:281) with homologous and/or orthologous amino acid sequences gi|11994583 (SEQ ID NO:282), CeresClone:1021269 (SEQ ID NO:283), CeresClone:592400 (SEQ ID NO:284), CeresClone:302213 (SEQ ID NO:285), and gi|50900102 (SEQ ID NO:286). The consensus sequence determined by the alignment is set forth.

FIG. 23 is an alignment of the amino acid sequence of Lead cDNA ID 23376150 (SEQ ID NO:288) with homologous and/or orthologous amino acid sequences gi|32362301 (SEQ ID NO:289), gi|8569103 (SEQ ID NO:290), CeresClone:597353 (SEQ ID NO:291), CeresClone:244954 (SEQ ID NO:292), gi|34105719 (SEQ ID NO:294), gi|34912214 (SEQ ID NO:295), CeresClone:292556 (SEQ ID NO:296), CeresClone:241094 (SEQ ID NO:298), and CeresClone:727806 (SEQ ID NO:299). The consensus sequence determined by the alignment is set forth.

FIG. 24 is an alignment of the amino acid sequence of Lead cDNA ID 23649144 (SEQ ID NO:301) with homologous and/or orthologous amino acid sequences gi|22137220 (SEQ ID NO:302), CeresClone:460973 (SEQ ID NO:303), CeresClone:464226 (SEQ ID NO:304), gi|50915436 (SEQ ID NO:305), CeresClone:1069366 (SEQ ID NO:306), and gi|50915434 (SEQ ID NO:307). The consensus sequence determined by the alignment is set forth.

FIG. 25 is an alignment of the amino acid sequence of Lead cDNA ID 23370269 (SEQ ID NO:309) with homologous and/or orthologous amino acid sequences CeresClone:38635 (SEQ ID NO:310), CeresClone:1375513 (SEQ ID NO:313), CeresClone:1242841 (SEQ ID NO:314), gi|12651665 (SEQ ID NO:315), gi|50939155 (SEQ ID NO:317), CeresClone:1063922 (SEQ ID NO:318), gi|62701860 (SEQ ID NO:319), CeresClone:293659 (SEQ ID NO:320), and CeresClone:1372772 (SEQ ID NO:321). The consensus sequence determined by the alignment is set forth.

FIG. 26 is an alignment of the amino acid sequence of Lead cDNA ID 23420310 (SEQ ID NO:325) with homologous and/or orthologous amino acid sequences gi|10177159 (SEQ ID NO:326), CeresClone:853230 (SEQ ID NO:327), gi|57899525 (SEQ ID NO:328), CeresClone:892520 (SEQ ID NO:330), and CeresClone:303140 (SEQ ID NO:331). The consensus sequence determined by the alignment is set forth.

FIG. 27 is an alignment of the amino acid sequence of Lead cDNA ID 23764087 (SEQ ID NO:333) with homologous and/or orthologous amino acid sequences gi|34910442 (SEQ ID NO:334), gi|45510867 (SEQ ID NO:335), gi|8777442 (SEQ ID NO:336), CeresClone:1242960 (SEQ ID NO:339), gi|6635379 (SEQ ID NO:340), CeresClone:530281 (SEQ ID NO:341), and gi|13924516 (SEQ ID NO:343). The consensus sequence determined by the alignment is set forth.

FIG. 28 is an alignment of the amino acid sequence of Lead cDNA ID 23460392 (SEQ ID NO:345) with homologous and/or orthologous amino acid sequences gi|51971865 (SEQ ID NO:346), gi|7268798 (SEQ ID NO:347), and CeresClone:783489 (SEQ ID NO:348). The consensus sequence determined by the alignment is set forth.

FIG. 29 is an alignment of the amino acid sequence of Lead cDNA ID 23419606 (SEQ ID NO:350) with homologous and/or orthologous amino acid sequence CeresClone:2347 (SEQ ID NO:352). The consensus sequence determined by the alignment is set forth.

FIG. 30 is an alignment of the amino acid sequence of Lead cDNA ID 23740209 (SEQ ID NO:356) with homologous and/or orthologous amino acid sequences gi|50940237 (SEQ ID NO:357), CeresClone:617111 (SEQ ID NO:358), CeresClone:207075 (SEQ ID NO:359), gi|21554154 (SEQ ID NO:360), gi|9759080 (SEQ ID NO:361), and CeresClone:471377 (SEQ ID NO:362). The consensus sequence determined by the alignment is set forth.

FIG. 31 is an alignment of the amino acid sequence of Lead cDNA ID 23374089 (SEQ ID NO:364) with homologous and/or orthologous amino acid sequences gi|50726625 (SEQ ID NO:365) and CeresClone:755158 (SEQ ID NO:366). The consensus sequence determined by the alignment is set forth.

FIG. 32 is an alignment of the amino acid sequence of Lead cDNA ID 23666854 (SEQ ID NO:370) with homologous and/or orthologous amino acid sequences gi|22136722 (SEQ ID NO:373) and gi|7578881 (SEQ ID NO:374). The consensus sequence determined by the alignment is set forth.

FIG. 33 is an alignment of the amino acid sequence of Lead cDNA ID 23662829 (SEQ ID NO:376) with homologous and/or orthologous amino acid sequences CeresClone:12573 (SEQ ID NO:377) and CeresClone:246144 (SEQ ID NO:380). The consensus sequence determined by the alignment is set forth.

FIG. 34 is an alignment of the amino acid sequence of Lead cDNA ID 23698996 (SEQ ID NO:382) with homologous and/or orthologous amino acid sequences gi|50906419 (SEQ ID NO:383)_{y} gi|15220810 (SEQ ID NO:384), and CeresClone:275358 (SEQ ID NO:385). The consensus sequence determined by the alignment is set forth.

FIG. 35 is an alignment of the amino acid sequence of Lead cDNA ID 23369491 (SEQ ID NO:387) with homologous and/or orthologous amino acid sequences CeresClone:463738 (SEQ ID NO:388), gi|50923675 (SEQ ID NO:389), and CeresClone:1213577 (SEQ ID NO:390). The consensus sequence determined by the alignment is set forth.

FIG. 36 is an alignment of the amino acid sequence of Lead cDNA ID 23384563 (SEQ ID NO:392) with homologous and/or orthologous amino acid sequences CeresClone:14909 (SEQ ID NO:393), CeresClone:33126 (SEQ ID NO:394), CeresClone:1338585 (SEQ ID NO:395), gi|39653273 (SEQ ID NO:396), CeresClone:276776 (SEQ ID NO:397), CeresClone:1535974 (SEQ ID NO:398), and CeresClone:240510 (SEQ ID NO:399). The consensus sequence determined by the alignment is set forth.

FIG. 37 is an alignment of the amino acid sequence of Lead cDNA ID 23389848 (SEQ ID NO:401) with homologous and/or orthologous amino acid sequences CeresClone:1388526 (SEQ ID NO:402), gi|55775124 (SEQ ID NO:403), CeresClone:477450 (SEQ ID NO:404), gi|34897896 (SEQ ID NO:405), CeresClone:700178 (SEQ ID NO:406), and gi|48209876 (SEQ ID NO:407). The consensus sequence determined by the alignment is set forth.

FIG. 38 is an alignment of the amino acid sequence of Lead cDNA ID 23384591 (SEQ ID NO:411) with homologous and/or orthologous amino acid sequences gi|9663025 (SEQ ID NO:412), CeresClone:305349 (SEQ ID NO:413), CeresClone:220215 (SEQ ID NO:414), gi|50945933 (SEQ ID NO:415), gi|52077258 (SEQ ID NO:416), and CeresClone:246718 (SEQ ID NO:417). The consensus sequence determined by the alignment is set forth.

FIG. 39 is an alignment of the amino acid sequence of Lead cDNA ID 23382112 (SEQ ID NO:419) with homologous and/or orthologous amino acid sequences gi|15293163 (SEQ ID NO:420), gi|34902154 (SEQ ID NO:421), CeresClone:363807 (SEQ ID NO:422), gi|62546183 (SEQ ID NO:423), gi|15148914 (SEQ ID NO:424), gi|56744294 (SEQ ID NO:425), gi|56785066 (SEQ ID NO:428), gi|51702424 (SEQ ID NO:429), gi|52353038 (SEQ ID NO:430), gi|21105748 (SEQ ID NO:431), and gi|4218535 (SEQ ID NO:432). The consensus sequence determined by the alignment is set forth.

FIG. 40 is an alignment of the amino acid sequence of Lead cDNA ID 23389418 (SEQ ID NO:434) with homologous and/or orthologous amino acid sequences CeresClone:942980 (SEQ ID NO:435), CeresClone:1265097 (SEQ ID NO:436), CeresClone:571184 (SEQ ID NO:437), CeresClone:1052457 (SEQ ID NO:438), CeresClone:1609912 (SEQ ID NO:439), CeresClone:323551 (SEQ ID NO:440), gi|57117314 (SEQ ID NO:441), gi|50928191 (SEQ ID NO:442), gi|50253143 (SEQ ID NO:443), gi|23451086 (SEQ ID NO:444), gi|38228693 (SEQ ID NO:445), gi|37901055 (SEQ ID NO:446), gi|20340241 (SEQ ID NO:447), and gi|20152976 (SEQ ID NO:448). The consensus sequence determined by the alignment is set forth.

FIG. 41 is an alignment of the amino acid sequence of Lead cDNA ID 23374668 (SEQ ID NO:450) with homologous and/or orthologous amino acid sequences gi|10177389 (SEQ ID NO:451), CeresClone:463247 (SEQ ID NO:452), gi|53791916 (SEQ ID NO:453), CeresClone:265056 (SEQ ID NO:454), CeresClone:336108 (SEQ ID NO:455), and CeresClone:906800 (SEQ ID NO:456). The consensus sequence determined by the alignment is set forth.

FIG. 42 is an alignment of the amino acid sequence of Lead cDNA ID 23365920 (SEQ ID NO:458) with homologous and/or orthologous amino acid sequences gi|5616313 (SEQ ID NO:459), CeresClone:751992 (SEQ ID NO:460), CeresClone:833872 (SEQ ID NO:461), gi|62901482 (SEQ ID NO:462), gi|34906988 (SEQ ID NO:463), and CeresClone:1579587 (SEQ ID NO:464). The consensus sequence determined by the alignment is set forth.

FIG. 43 is an alignment of the amino acid sequence of Lead cDNA ID 23370421 (SEQ ID NO: 466) with homologous and/or orthologous amino acid sequences CeresClone:870962 (SEQ ID NO:467), CeresClone:562536 (SEQ ID NO:468), CeresClone:1032823 (SEQ ID NO:469), and CeresClone:314156 (SEQ ID NO:470). The consensus sequence determined by the alignment is set forth.

FIG. 44 is an alignment of the amino acid sequence of Lead cDNA ID 23783423 (SEQ ID NO:472) with homologous and/or orthologous amino acid sequences gi|9367307 (SEQ ID NO:473), gi|62510920 (SEQ ID NO:474), gi|28630957 (SEQ ID NO:475), gi|6175371 (SEQ ID NO:476), gi|33309864 (SEQ ID NO:477), gi|6467974 (SEQ ID NO:478), gi|1483232 (SEQ ID NO:479), CeresClone:510092 (SEQ ID NO:481), gi|29372764 (SEQ ID NO:482), gi|33355661 (SEQ ID NO:483), gi|30090030 (SEQ ID NO:484), gi|58423002 (SEQ ID NO:486), gi|33391153 (SEQ ID NO:487), and gi|39843110 (SEQ ID NO:488). The consensus sequence determined by the alignment is set forth.

FIG. 45 is an alignment of the amino acid sequence of Lead cDNA ID 23538950 (5109B2; SEQ ID NO:494) with homologous and/or orthologous amino acid sequences CeresClone:567184 (SEQ ID NO:496), CeresClone:967417 (SEQ ID NO:497), CeresClone:1360570 (SEQ ID NO:498), CeresClone:701370 (SEQ ID NO:499), gi|5031281 (SEQ ID NO:500), gi|35187687 (SEQ ID NO:501), gi|34910634 (SEQ ID NO:503), and CeresClone:1609861 (SEQ ID NO:504). The consensus sequence determined by the alignment is set forth.

FIG. 46 is an alignment of the amino acid sequence of Lead cDNA ID 24373996 (5109E11; SEQ ID NO:506) with homologous and/or orthologous amino acid sequences CeresClone:563014 (SEQ ID NO:507), gi|22795037 (SEQ ID NO:508), gi|41059804 (SEQ ID NO:509), CeresClone:883322 (SEQ ID NO:511), CeresClone:244940 (SEQ ID NO:512), and gi|50926652 (SEQ ID NO:514). The consensus sequence determined by the alignment is set forth.

FIG. 47 is an alignment of the amino acid sequence of Lead cDNA ID 23539673 (5110C6; SEQ ID NO:516) with homologous and/or orthologous amino acid sequences CeresClone:477085 (SEQ ID NO:517), CeresClone:387243 (SEQ ID NO:518), and gi|50898950 (SEQ ID NO:520). The consensus sequence determined by the alignment is set forth.

FIG. 48 is an alignment of the amino acid sequence of Lead cDNA ID 23357846 (SEQ ID NO:523) with homologous and/or orthologous amino acid sequences CeresClone:539578 (SEQ ID NO:524), CeresClone:596339 (SEQ ID NO:525), gi|6018699 (SEQ ID NO:529), and gi|50725042 (SEQ ID NO:530). The consensus sequence determined by the alignment is set forth.

FIG. 49 is an alignment of the amino acid sequence of Lead cDNA ID 12680548 (SEQ ID NO:532) with homologous and/or orthologous amino acid sequences gi|62632894 (SEQ ID NO:533), CeresClone:1065387 (SEQ ID NO:534), gi|30523250 (SEQ ID NO:537), gi|30523252 (SEQ ID NO:538), gi|30523362 (SEQ ID NO:540), CeresClone:1091989 (SEQ ID NO:541), gi|30523360 (SEQ ID NO:543), and gi|30523366 (SEQ ID NO:546). The consensus sequence determined by the alignment is set forth.

FIG. 50 is an alignment of the amino acid sequence of Lead cDNA ID 23357564 (SEQ ID NO:548) with homologous and/or orthologous amino acid sequences CeresClone:11615 (SEQ ID NO:549), gi|17104699 (SEQ ID NO:550), CeresClone:1027567 (SEQ ID NO:551), CeresClone:1060767 (SEQ ID NO:552), CeresClone:1034616 (SEQ ID NO:553), CeresClone:1058733 (SEQ ID NO:554), gi|2894109 (SEQ ID NO:555), CeresClone:782784 (SEQ ID NO:556), gi|18645 (SEQ ID NO:557), CeresClone:721511 (SEQ ID NO:558), CeresClone:641329 (SEQ ID NO:559), gi|7446213 (SEQ ID NO:560), and gi|1052956 (SEQ ID NO:561). The consensus sequence determined by the alignment is set forth.

FIG. 51 is an alignment of the amino acid sequence of Lead cDNA ID 23660778 (5109A5; SEQ ID NO:565) with homologous and/or orthologous amino acid sequences gi|50251990 (SEQ ID NO:566), CeresClone:304939 (SEQ ID NO:567), and CeresClone:569545 (SEQ ID NO:568). The consensus sequence determined by the alignment is set forth.

FIG. 52 is an alignment of the amino acid sequence of Lead cDNA ID 23653450 (5109C6; SEQ ID NO:574) with homologous and/or orthologous amino acid sequences gi|50938747 (SEQ ID NO:575), CeresClone:458156 (SEQ ID NO:576), and CeresClone:918824 (SEQ ID NO:577). The consensus sequence determined by the alignment is set forth.

FIG. 53 is an alignment of the amino acid sequence of Lead cDNA ID 23467847 (5109D1; SEQ ID NO:579) with homologous and/or orthologous amino acid sequences gi|63252923 (SEQ ID NO:580), CeresClone:363807 (SEQ ID NO:581), gi|58013003 (SEQ ID NO:582), gi|52353038 (SEQ ID NO:583), gi|34902154 (SEQ ID NO:584), gi|21105748 (SEQ ID NO:585), gi|66275772 (SEQ ID NO:586), gi|53749460 (SEQ ID NO:587), and gi|15148914 (SEQ ID NO:588). The consensus sequence determined by the alignment is set forth.

FIG. 54 is an alignment of the amino acid sequence of Lead 5109E2 (cDNA ID 23553534; SEQ ID NO:593) with homologous and/or orthologous amino acid sequences CeresClone:956332 (SEQ ID NO:594), CeresClone:1049567 (SEQ ID NO:595), gi|34898438 (SEQ ID NO:596), and CeresClone:280534 (SEQ ID NO:597). The consensus sequence determined by the alignment is set forth.

FIG. 55 is an alignment of the amino acid sequence of Lead cDNA ID 23498294 (5109F2; SEQ ID NO:599) with homologous and/or orthologous amino acid sequences CeresClone:957882 (SEQ ID NO:600), gi|50726297 (SEQ ID NO:601), CeresClone:739665 (SEQ ID NO:602), CeresClone:294374 (SEQ ID NO:603), CeresClone:656020 (SEQ ID NO:605), and gi|3334756 (SEQ ID NO:606). The consensus sequence determined by the alignment is set forth.

FIG. 56 is an alignment of the amino acid sequence of Lead cDNA ID 23529931 (5109H10; SEQ ID NO:608) with homologous and/or orthologous amino acid sequences CeresClone:1021260 (SEQ ID NO:609) and CeresClone:239775 (SEQ ID NO:610). The consensus sequence determined by the alignment is set forth.

FIG. 57 is an alignment of the amino acid sequence of Lead cDNA ID 23498685 (5109H3; SEQ ID NO:613) with homologous and/or orthologous amino acid sequences gi|52077327 (SEQ ID NO:614), CeresClone:1044645 (SEQ ID NO:615), CeresClone:1548279 (SEQ ID NO:616), and CeresClone:727056 (SEQ ID NO:617). The consensus sequence determined by the alignment is set forth.

FIG. 58 is an alignment of the amino acid sequence of Lead cDNA ID 23515088 (SEQ ID NO:619) with homologous and/or orthologous amino acid sequences gi|50916012 (SEQ ID NO:620), gi|861091 (SEQ ID NO:621), gi|2346972 (SEQ ID NO:622), CeresClone:519630 (SEQ ID NO:623), gi|7228329 (SEQ ID NO:624), gi|2981169 (SEQ ID NO:625), gi|55734108 (SEQ ID NO:626), gi|33331578 (SEQ ID NO:627), gi|51871855 (SEQ ID NO:628), and gi|2058506 (SEQ ID NO:629). The consensus sequence determined by the alignment is set forth.

FIG. 59 is an alignment of the amino acid sequence of Lead cDNA ID 24375036 (5110A2; SEQ ID NO:632) with homologous and/or orthologous amino acid sequences CeresClone:971843 (SEQ ID NO:633), CeresClone:361557 (SEQ ID NO:634), and CeresClone:535370 (SEQ ID NO:635). The consensus sequence determined by the alignment is set forth.

FIG. 60 is an alignment of the amino acid sequence of Lead cDNA ID 23544992 (SEQ ID NO:639) with homologous and/or orthologous amino acid sequences gi|1362020 (SEQ ID NO:640), gi|51536147 (SEQ ID NO:641), CeresClone:1060169 (SEQ ID NO:642), CeresClone:1461776 (SEQ ID NO:645), and gi|18390109 (SEQ ID NO:646). The consensus sequence determined by the alignment is set forth.

FIG. 61 is an alignment of the amino acid sequence of Lead cDNA ID 23517564 (5110B2; SEQ ID NO:648) with homologous and/or orthologous amino acid sequences CeresClone:936276 (SEQ ID NO:649) and CeresClone:234834 (SEQ ID NO:650). The consensus sequence determined by the alignment is set forth.

FIG. 62 is an alignment of the amino acid sequence of Lead cDNA ID 23502669 (5110B7; SEQ ID NO:652) with homologous and/or orthologous amino acid sequences gi|20502805 (SEQ ID NO:653), gi|34912988 (SEQ ID NO:654), and gi|20467991 (SEQ ID NO:655). The consensus sequence determined by the alignment is set forth.

FIG. 63 is an alignment of the amino acid sequence of Lead cDNA ID 23515246 (5110D5; SEQ ID NO:659) with homologous and/or orthologous amino acid sequences gi|50911537 (SEQ ID NO:660) and CeresClone:788036 (SEQ ID NO:662). The consensus sequence determined by the alignment is set forth.

FIG. 64 is an alignment of the amino acid sequence of Lead cDNA ID 24380616 (5110E4; SEQ ID NO:664) with homologous and/or orthologous amino acid sequences CeresClone:280261 (SEQ ID NO:665), gi|50947859 (SEQ ID NO:666), and CeresClone:1325022 (SEQ ID NO:669). The consensus sequence determined by the alignment is set forth.

FIG. 65 is an alignment of the amino acid sequence of Lead cDNA ID 23467433 (5110E7; SEQ ID NO:674) with homologous and/or orthologous amino acid sequences CeresClone:265352 (SEQ ID NO:676) and gi|50928925 (SEQ ID NO:677). The consensus sequence determined by the alignment is set forth.

FIG. 66 is an alignment of the amino acid sequence of Lead cDNA ID 23524514 (5110F4; SEQ ID NO:686) with homologous and/or orthologous amino acid sequences CeresClone:566396 (SEQ ID NO:690), gi|5139697 (SEQ ID NO:691), and gi|53748471 (SEQ ID NO:693). The consensus sequence determined by the alignment is set forth.

FIG. 67 is an alignment of the amino acid sequence of Lead cDNA ID 23503210 (5110G1; SEQ ID NO:695) with homologous and/or orthologous amino acid sequence CeresClone:654820 (SEQ ID NO:696). The consensus sequence determined by the alignment is set forth.

FIG. 68 is an alignment of the amino acid sequence of Lead cDNA ID 23494809 (5110G5; SEQ ID NO:698) with homologous and/or orthologous amino acid sequence gi|32455231 (SEQ ID NO:699). The consensus sequence determined by the alignment is set forth.

FIG. 69 is an alignment of the amino acid sequence of Lead cDNA ID 23740916 (SEQ ID NO:703) with homologous and/or orthologous amino acid sequences CeresClone:114879 (SEQ ID NO:705), CeresClone:524672 (SEQ ID NO:707), CeresClone:570129 (SEQ ID NO:708), and gi|53793441 (SEQ ID NO:709). The consensus sequence determined by the alignment is set forth.

FIG. 70 is an alignment of the amino acid sequence of Lead cDNA ID 23363175 (SEQ ID NO:711) with homologous and/or orthologous amino acid sequences gi|34896098 (SEQ ID NO:712), CeresClone:930868 (SEQ ID NO:713), and gi|50949055 (SEQ ID NO:714). The consensus sequence determined by the alignment is set forth.

FIG. 71 is an alignment of the amino acid sequence of Lead cDNA ID 23421865 (SEQ ID NO:716) with homologous and/or orthologous amino acid sequences gi|27808566 (SEQ ID NO:717), CeresClone:710195 (SEQ ID NO:718), and CeresClone:222899 (SEQ ID NO:719). The consensus sequence determined by the alignment is set forth.

FIG. 72 is an alignment of the amino acid sequence of Lead cDNA ID 23417641 (SEQ ID NO:721) with homologous and/or orthologous amino acid sequences CeresClone:982869 (SEQ ID NO:722), gi|20258977 (SEQ ID NO:723), CeresClone:538662 (SEQ ID NO:724), gi|18874263 (SEQ ID NO:725), gi|56605378 (SEQ ID NO:726), gi|51557078 (SEQ ID NO:727), CeresClone:833986 (SEQ ID NO:729), and gi|53749253 (SEQ ID NO:730). The consensus sequence determined by the alignment is set forth.

FIG. 73 is an alignment of the amino acid sequence of Lead cDNA ID 23751471 (SEQ ID NO:732) with homologous and/or orthologous amino acid sequences CeresClone:212540 (SEQ ID NO:733), gi|50939031 (SEQ ID NO:734), CeresClone:700212 (SEQ ID NO:735), CeresClone:1341109 (SEQ ID NO:736), CeresClone: 16467 (SEQ ID NO:740), and CeresClone:36048 (SEQ ID NO:746). The consensus sequence determined by the alignment is set forth.

FIG. 74 is an alignment of the amino acid sequence of Lead cDNA ID 23773450 (SEQ ID NO:748) with homologous and/or orthologous amino acid sequences gi|50251892 (SEQ ID NO:750), gi|44888603 (SEQ ID NO:751), gi|3688591 (SEQ ID NO:752), gi|13958339 (SEQ ID NO:753), gi|28630959 (SEQ ID NO:754), gi|40644776 (SEQ ID NO:755), gi|47681319 (SEQ ID NO:756), gi|7544096 (SEQ ID NO:757), and gi|20385586 (SEQ ID NO:758). The consensus sequence determined by the alignment is set forth.

FIG. 75 is an alignment of the amino acid sequence of Lead cDNA ID 23760303 (SEQ ID NO:760) with homologous and/or orthologous amino acid sequences gi|50947859 (SEQ ID NO:761), CeresClone:1325022 (SEQ ID NO:763), and CeresClone:1343742 (SEQ ID NO:764). The consensus sequence determined by the alignment is set forth.

FIG. 76 is an alignment of the amino acid sequence of Lead cDNA ID 23772039 (SEQ ID NO:766) with homologous and/or orthologous amino acid sequence CeresClone:864432 (SEQ ID NO:767). The consensus sequence determined by the alignment is set forth.

FIG. 77 is an alignment of the amino acid sequence of Lead cDNA ID 23792467 (SEQ ID NO:769) with homologous and/or orthologous amino acid sequences gi|32470645 (SEQ ID NO:770), CeresClone:537360 (SEQ ID NO:771), gi|4835766 (SEQ ID NO:773), CeresClone:677527 (SEQ ID NO:774), and gi|4519671 (SEQ ID NO:775). The consensus sequence determined by the alignment is set forth.

FIG. 78 is an alignment of the amino acid sequence of Lead cDNA ID 23401404 (SEQ ID NO:777) with homologous and/or orthologous amino acid sequences gi|34910914 (SEQ ID NO:778), CeresClone:1064154 (SEQ ID NO:779), CeresClone:113582 (SEQ ID NO:780), gi|21536857 (SEQ ID NO:781), gi|2894109 (SEQ ID NO:782), CeresClone:686294 (SEQ ID NO:783), gi|436424 (SEQ ID NO:784), gi|950053 (SEQ ID NO:785), gi|7446213 (SEQ ID NO:786), gi|729737 (SEQ ID NO:787), gi|7446231 (SEQ ID NO:788), gi|729736 (SEQ ID NO:789), and gi|1052956 (SEQ ID NO:790). The consensus sequence determined by the alignment is set forth.

FIG. 79 is an alignment of the amino acid sequence of Lead cDNA ID 23365746 (SEQ ID NO:792) with homologous and/or orthologous amino acid sequences gi|34907424 (SEQ ID NO:793), CeresClone:475016 (SEQ ID NO:794), and CeresClone:1571937 (SEQ ID NO:795). The consensus sequence determined by the alignment is set forth.

FIG. 80 is an alignment of the amino acid sequence of Lead cDNA ID 23765347 (SEQ ID NO:797) with homologous and/or orthologous amino acid sequences gi|50944571 (SEQ ID NO:798), CeresClone:239069 (SEQ ID NO:799), CeresClone:677527 (SEQ ID NO:800), CeresClone:242603 (SEQ ID NO:802), CeresClone:38327 (SEQ ID NO:803), CeresClone:463968 (SEQ ID NO:805), CeresClone:6626 (SEQ ID NO:806), CeresClone:581430 (SEQ ID NO:809), and gi|32470645 (SEQ ID NO:810). The consensus sequence determined by the alignment is set forth.

FIG. 81 is an alignment of the amino acid sequence of Lead cDNA ID 23768927 (SEQ ID NO:812) with homologous and/or orthologous amino acid sequences gi|51964894_T (SEQ ID NO:816), gi|16974539_T (SEQ ID NO:817), and CeresClone:557659_T (SEQ ID NO:818). The consensus sequence determined by the alignment is set forth.

FIG. 82 is an alignment of the amino acid sequence of Lead cDNA ID 23495742 (5109D9; SEQ ID NO:822) with homologous and/or orthologous amino acid sequences gi|57999638 (SEQ ID NO:823), CeresClone:1067477 (SEQ ID NO:824), gi|42795299 (SEQ ID NO:825), and CeresClone:244495 (SEQ ID NO:826). The consensus sequence determined by the alignment is set forth.

FIG. 83 is an alignment of the amino acid sequence of Lead cDNA ID 23523867 (5109E10; SEQ ID NO:828) with homologous and/or orthologous amino acid sequences CeresClone:955910 (SEQ ID NO:829), gi|50939215 (SEQ ID NO:830), gi|50939195 (SEQ ID NO:831), and CeresClone:333937 (SEQ ID NO:832). The consensus sequence determined by the alignment is set forth.

FIG. 84 is an alignment of the amino acid sequence of Lead cDNA ID 23516633 (5109E3; SEQ ID NO:834) with homologous and/or orthologous amino acid sequences gi|6899920 (SEQ ID NO:835), gi|20269055 (SEQ ID NO:836), and CeresClone:675127 (SEQ ID NO:838). The consensus sequence determined by the alignment is set forth.

FIG. 85 is an alignment of the amino acid sequence of Lead cDNA ID 23505323 (5110B10; SEQ ID NO:840) with homologous and/or orthologous amino acid sequences CeresClone:300033 (SEQ ID NO:842) and CeresClone:557223 (SEQ ID NO:843). The consensus sequence determined by the alignment is set forth.

FIG. 86 is an alignment of the amino acid sequence of Lead cDNA ID 23492765 (5110C3; SEQ ID NO:845) with homologous and/or orthologous amino acid sequences CeresClone:669185 (SEQ ID NO:846), CeresClone:381106 (SEQ ID NO:847), and gi|55297106 (SEQ ID NO:848). The consensus sequence determined by the alignment is set forth.

FIG. 87 is an alignment of the amino acid sequence of Lead cDNA ID 23486285 (5110C4; SEQ ID NO:851) with homologous and/or orthologous amino acid sequences CeresClone:100484 (SEQ ID NO:852), CeresClone:847458 (SEQ ID NO:853), and gi|50909371 (SEQ ID NO:854). The consensus sequence determined by the alignment is set forth.

FIG. 88 is an alignment of the amino acid sequence of Lead cDNA ID 23499964 (5110D4; SEQ ID NO:856) with homologous and/or orthologous amino acid sequences CeresClone:546084 (SEQ ID NO:857), CeresClone:1567551 (SEQ ID NO:858), gi|50428739 (SEQ ID NO:859), and CeresClone:576107 (SEQ ID NO:866). The consensus sequence determined by the alignment is set forth.

FIG. 89 is an alignment of the amino acid sequence of Lead cDNA ID 23397999 (SEQ ID NO:874) with homologous and/or orthologous amino acid sequences CeresClone:374770 (SEQ ID NO:875), gi|21717332 (SEQ ID NO:876), gi|11181612 (SEQ ID NO:877), gi|28894445 (SEQ ID NO:878), gi|20259679 (SEQ ID NO:879), gi|42570959 (SEQ ID NO:880), gi|25354653 (SEQ ID NO:881), gi|34900512 (SEQ ID NO:882), gi|13173164 (SEQ ID NO:883), gi|51100730 (SEQ ID NO:884), gi|5081557 (SEQ ID NO:885), gi|53801434 (SEQ ID NO:886), and gi|53830031 (SEQ ID NO:887). The consensus sequence determined by the alignment is set forth.

FIG. 90 is an alignment of the amino acid sequence of Lead cDNA ID 23556617 (SEQ ID NO:889) with homologous and/or orthologous amino acid sequences gi|23194453 (SEQ ID NO:890), gi|60100358 (SEQ ID NO:891), gi|3646326 (SEQ ID NO:892), CeresClone:1044034 (SEQ ID NO:893), gi|4103342 (SEQ ID NO:894), gi|20385590 (SEQ ID NO:896), gi|27763670 (SEQ ID NO:897), gi|57157565 (SEQ ID NO:898), gi|42794560 (SEQ ID NO:899), gi|29467048 (SEQ ID NO:900), gi|48727598 (SEQ ID NO:901), gi|21955182 (SEQ ID NO:902), and g|1568513 (SEQ ID NO:903). The consensus sequence determined by the alignment is set forth.

FIG. 91 is an alignment of the amino acid sequence of Lead cDNA ID 23557650 (SEQ ID NO:906) with homologous and/or orthologous amino acid sequences CeresClone:1033993 (SEQ ID NO:907), CeresClone:703180 (SEQ ID NO:908), CeresClone:560681 (SEQ ID NO:909), CeresClone:560948 (SEQ ID NO:911), CeresClone:653656 (SEQ ID NO:913), gi|50929085 (SEQ ID NO:915), gi|50912765 (SEQ ID NO:916), CeresClone:503296 (SEQ ID NO:917), and CeresClone:486120 (SEQ ID NO:918). The consensus sequence determined by the alignment is set forth.

FIG. 92 is an alignment of the amino acid sequence of Lead cDNA ID 23385560 (SEQ ID NO:921) with homologous and/or orthologous amino acid sequences CeresClone:1014844 (SEQ ID NO:922), gi|18857720 (SEQ ID NO:923), gi|1234900 (SEQ ID NO:924), CeresClone:527278 (SEQ ID NO:925), gi|1149535 (SEQ ID NO:926), CeresClone:514259 (SEQ ID NO:927), gi|8919876 (SEQ ID NO:928), and gi|992598 (SEQ ID NO:929). The consensus sequence determined by the alignment is set forth.

FIG. 93 is an alignment of the amino acid sequence of Lead cDNA ID 23389966 (SEQ ID NO:931) with homologous and/or orthologous amino acid sequences gi|20197615 (SEQ ID NO:932), CeresClone:18215 (SEQ ID NO:933), CeresClone:105261 (SEQ ID NO:935), CeresClone:24667 (SEQ ID NO:938), CeresClone:118878 (SEQ ID NO:940), CeresClone:12459 (SEQ ID NO:941), and CeresClone:1354021 (SEQ ID NO:942). The consensus sequence determined by the alignment is set forth.

FIG. 94 is an alignment of the amino acid sequence of Lead cDNA ID 23766279 (SEQ ID NO:946) with homologous and/or orthologous amino acid sequences gi|57283093 (SEQ ID NO:947), gi|9367234 (SEQ ID NO:951), CeresClone:354084 (SEQ ID NO:952), gi|10944320 (SEQ ID NO:954), gi|33943515 (SEQ ID NO:956), gi|6652756 (SEQ ID NO:958), gi|16549058 (SEQ ID NO:959), gi|30983948 (SEQ ID NO:960), gi|30575602 (SEQ ID NO:961), and gi|22779230 (SEQ ID NO:962). The consensus sequence determined by the alignment is set forth.

FIG. 95 is an alignment of the amino acid sequence of Lead cDNA ID 23746932 (SEQ ID NO:964) with homologous and/or orthologous amino acid sequences gi|29372750 (SEQ ID NO:965), gi|62148942 (SEQ ID NO:966), and gi|9367234 (SEQ ID NO:971). The consensus sequence determined by the alignment is set forth.

FIG. 96 is an alignment of the amino acid sequence of Lead cDNA ID 23380615 (SEQ ID NO:973) with homologous and/or orthologous amino acid sequences CeresClone:7559 (SEQ ID NO:974), gi|52140010 (SEQ ID NO:975), CeresClone:844350 (SEQ ID NO:976), gi|52140009 (SEQ ID NO:977), CeresClone:298172 (SEQ ID NO:978), gi|52140013 (SEQ ID NO:979), CeresClone:541062 (SEQ ID NO:980), and gi|52140015 (SEQ ID NO:981). The consensus sequence determined by the alignment is set forth.

FIG. 97 is an alignment of the amino acid sequence of Lead cDNA ID 23366147 (SEQ ID NO:983) with homologous and/or orthologous amino acid sequences CeresClone:608818 (SEQ ID NO:984), CeresClone:1559765 (SEQ ID NO:985), gi|115840 (SEQ ID NO:986), and CeresClone:638098 (SEQ ID NO:990). The consensus sequence determined by the alignment is set forth.

FIG. 98 is an alignment of the amino acid sequence of Lead cDNA ID 23416775 (SEQ ID NO:992) with homologous and/or orthologous amino acid sequences CeresClone:1091297 (SEQ ID NO:993), gi|33324520 (SEQ ID NO:994), gi|55741382 (SEQ ID NO:995), CeresClone:471446 (SEQ ID NO:996), CeresClone:472054 (SEQ ID NO:997), CeresClone:1050656 (SEQ ID NO:998), and gi|31324058 (SEQ ID NO:999). The consensus sequence determined by the alignment is set forth.

FIG. 99 is an alignment of the amino acid sequence of Lead cDNA ID 23359888 (SEQ ID NO:1001) with homologous and/or orthologous amino acid sequences CeresClone:30700 (SEQ ID NO:1002), gi|19698881 (SEQ ID NO:1004), gi|19697 (SEQ ID NO:1005), gi|475216 (SEQ ID NO:1007), gi|2119932 (SEQ ID NO:1010), gi|2119933 (SEQ ID NO:1014), gi|485951 (SEQ ID NO:1015), and gi|25809054 (SEQ ID NO:1017). The consensus sequence determined by the alignment is set forth.

FIG. 100 is an alignment of the amino acid sequence of Lead cDNA ID 23385230 (SEQ ID NO:1019) with homologous and/or orthologous amino acid sequences gi|25405956 (SEQ ID NO:1020), gi|30694486 (SEQ ID NO:1021), CeresClone:354956 (SEQ ID NO:1022), gi|22854970 (SEQ ID NO:1023), and gi|22854950 (SEQ ID NO:1024). The consensus sequence determined by the alignment is set forth.

FIG. 101 is an alignment of the amino acid sequence of Lead cDNA ID 23359443 (SEQ ID NO:1026) with homologous and/or orthologous amino acid sequences gi|1806261 (SEQ ID NO:1027), gi|542187 (SEQ ID NO:1029), gi|15865782 (SEQ ID NO:1031), CeresClone:235570 (SEQ ID NO:1032), gi|16797791 (SEQ ID NO:1033), CeresClone:295738 (SEQ ID NO:1035), gi|34897226 (SEQ ID NO:1036), gi|1869928 (SEQ ID NO:1037), gi|1144536 (SEQ ID NO:1038), and gi|4115746 (SEQ ID NO:1039). The consensus sequence determined by the alignment is set forth.

FIG. 102 is an alignment of the amino acid sequence of Lead cDNA ID 23386664 (SEQ ID NO:1042) with homologous and/or orthologous amino acid sequences gi|14030607 (SEQ ID NO:1043), CeresClone:1090803 (SEQ ID NO:1045), CeresClone:1086365 (SEQ ID NO:1047), CeresClone:1323425 (SEQ ID NO:1048), CeresClone:373100 (SEQ ID NO:1050), gi|50251897 (SEQ ID NO:1051), gi|5107149 (SEQ ID NO:1052), gi|50928231 (SEQ ID NO:1053), CeresClone:584348 (SEQ ID NO:1055), and gi|5107157 (SEQ ID NO:1056). The consensus sequence determined by the alignment is set forth.

FIG. 103 is an alignment of the amino acid sequence of Lead cDNA ID 23371818 (SEQ ID NO:1058) with homologous and/or orthologous amino acid sequences gi|15810073 (SEQ ID NO:1059), CeresClone:285163 (SEQ ID NO:1060), gi|50906555 (SEQ ID NO:1061), gi|34909384 (SEQ ID NO:1062), gi|17976835 (SEQ ID NO:1063), gi|32396295 (SEQ ID NO:1064), gi|16610193 (SEQ ID NO:1065), and gi|20269057 (SEQ ID NO:1066). The consensus sequence determined by the alignment is set forth.

FIG. 104 is an alignment of the amino acid sequence of Lead cDNA ID 23471864 (SEQ ID NO:1068) with homologous and/or orthologous amino acid sequences CeresClone:647941 (SEQ ID NO:1069), CeresClone:1246527 (SEQ ID NO:1070), CeresClone:1306476 (SEQ ID NO:1071), and CeresClone:1259850 (SEQ ID NO:1072). The consensus sequence determined by the alignment is set forth.

FIG. 105 is an alignment of the amino acid sequence of Lead cDNA ID 23370870 (SEQ ID NO:1074) with homologous and/or orthologous amino acid sequences gi|47680447 (SEQ ID NO:1075), gi|1370140 (SEQ ID NO:1078), gi|20561 (SEQ ID NO:1079), gi|22266673 (SEQ ID NO:1081), gi|22266675 (SEQ ID NO:1082), gi|1732247 (SEQ ID NO:1083), gi|5139814 (SEQ ID NO:1084), and gi|6552361 (SEQ ID NO:1085). The consensus sequence determined by the alignment is set forth.

FIG. 106 is an alignment of the amino acid sequence of Lead cDNA ID 23361688 (SEQ ID NO:1087) with homologous and/or orthologous amino acid sequences CeresClone:280394 (SEQ ID NO:1088), gi|50945939 (SEQ ID NO:1089), gi|19073336 (SEQ ID NO:1090), gi|19073332 (SEQ ID NO:1091), CeresClone:1061835 (SEQ ID NO:1092), gi|19073330 (SEQ ID NO:1093), gi|13346188 (SEQ ID NO:1094), gi|6651292 (SEQ ID NO:1095), gi|1430846 (SEQ ID NO:1096), gi|34147926 (SEQ ID NO:1097), gi|50948253 (SEQ ID NO:1098), and gi|23343579 (SEQ ID NO:1100). The consensus sequence determined by the alignment is set forth.

FIG. 107 is an alignment of the amino acid sequence of Lead cDNA ID 23448883 (SEQ ID NO:1102) with homologous and/or orthologous amino acid sequences gi|21617978 (SEQ ID NO:104), gi|2829920 (SEQ ID NO:1105), CeresClone:1065387 (SEQ ID NO:1107), CeresClone:1091989 (SEQ ID NO:1110), gi|34591565 (SEQ ID NO:1112), gi|30523250 (SEQ ID NO:1113), gi|30523252 (SEQ ID NO:1114), and gi|45181459 (SEQ ID NO:1115). The consensus sequence determined by the alignment is set forth.

FIG. 108 is an alignment of the amino acid sequence of Lead cDNA ID 23389186 (SEQ ID NO:1119) with homologous and/or orthologous amino acid sequences CeresClone:625275 (SEQ ID NO:1120), CeresClone:1246429 (SEQ ID NO:1121), gi|37718893 (SEQ ID NO:1122), CeresClone:937503 (SEQ ID NO:1123), CeresClone:400568 (SEQ ID NO:1124), and CeresClone:1549251 (SEQ ID NO:1125). The consensus sequence determined by the alignment is set forth.

FIG. 109 is an alignment of the amino acid sequence of Lead cDNA ID 23380898 (SEQ ID NO:1127) with homologous and/or orthologous amino acid sequences CeresClone:13879 (SEQ ID NO:1128), gi|21553354 (SEQ ID NO:1129), CeresClone:158026 (SEQ ID NO:1130), CeresClone:1012104 (SEQ ID NO:1131), gi|1346180 (SEQ ID NO:1132), gi|1346181 (SEQ ID NO:1133), gi|17819 (SEQ ID NO:1134), gi|34851124 (SEQ ID NO:1135), and CeresClone:583672 (SEQ ID NO:1136). The consensus sequence determined by the alignment is set forth.

FIG. 110 is an alignment of the amino acid sequence of Lead cDNA ID 23383311 (SEQ ID NO:1138) with homologous and/or orthologous amino acid sequences CeresClone:659723 (SEQ ID NO:1139), CeresClone:953644 (SEQ ID NO:1140), CeresClone:1585988 (SEQ ID NO:1141), CeresClone:245683 (SEQ ID NO:1142), CeresClone:1283552 (SEQ ID NO:1143), CeresClone:272426 (SEQ ID NO:1144), and CeresClone:824827 (SEQ ID NO:1145). The consensus sequence determined by the alignment is set forth.

FIG. 111 is an alignment of the amino acid sequence of Lead cDNA ID 23384792 (SEQ ID NO:1147) with homologous and/or orthologous amino acid sequences CeresClone:467528 (SEQ ID NO:1148), gi|20269057 (SEQ ID NO:1149), gi|51964528 (SEQ ID NO:1150), gi|50915894 (SEQ ID NO:1151), gi|32396299 (SEQ ID NO:1152), gi|62120254 (SEQ ID NO:1153), gi|4887020 (SEQ ID NO:1154), gi|4887022 (SEQ ID NO:1155), and CeresClone:305337 (SEQ ID NO:1156). The consensus sequence determined by the alignment is set forth.

FIG. 112 is an alignment of the amino acid sequence of Lead cDNA ID 23360311 (SEQ ID NO:1158) with homologous and/or orthologous amino acid sequences CeresClone:627169 (SEQ ID NO:1159), gi|34914598 (SEQ ID NO:1160), CeresClone:1397168 (SEQ ID NO:1161), gi|50909895 (SEQ ID NO:1162), and CeresClone:704527 (SEQ ID NO:1163). The consensus sequence determined by the alignment is set forth.

FIG. 113 is an alignment of the amino acid sequence of Lead cDNA ID 23375896 (SEQ ID NO:1165) with homologous and/or orthologous amino acid sequences CeresClone:476024 (SEQ ID NO:1166), CeresClone:1017044 (SEQ ID NO:1167), CeresClone:230052 (SEQ ID NO:1168), and CeresClone:341096 (SEQ ID NO:1169). The consensus sequence determined by the alignment is set forth.

FIG. 114 is an alignment of the amino acid sequence of Lead cDNA ID 23376628 (SEQ ID NO:1171) with homologous and/or orthologous amino acid sequences CeresClone:636599 (SEQ ID NO:1172), gi|50934801 (SEQ ID NO:1173), gi|31712074 (SEQ ID NO:1174), CeresClone:696154 (SEQ ID NO:1175), and CeresClone:1554290 (SEQ ID NO:1176). The consensus sequence determined by the alignment is set forth.

FIG. 115 is an alignment of the amino acid sequence of Lead cDNA ID 23369842 (SEQ ID NO:1178) with homologous and/or orthologous amino acid sequences gi|8809670 (SEQ ID NO:1179), CeresClone:254065 (SEQ ID NO:1180), gi|38564314 (SEQ ID NO:1181), CeresClone:477450 (SEQ ID NO:1182), CeresClone:280814 (SEQ ID NO:1183), gi|55775124 (SEQ ID NO:1184), CeresClone:295114 (SEQ ID NO:1185), CeresClone:241340 (SEQ ID NO:1186), gi|32489377 (SEQ ID NO:1187), CeresClone:700178 (SEQ ID NO:1188), gi|50928853 (SEQ ID NO:1189), and gi|50918277 (SEQ ID NO:1190). The consensus sequence determined by the alignment is set forth.

FIG. 116 is an alignment of the amino acid sequence of Lead cDNA ID 23416869 (SEQ ID NO:1192) with homologous and/or orthologous amino acid sequences CeresClone:738705 (SEQ ID NO:1193), CeresClone:892214 (SEQ ID NO:1194), gi|50913251 (SEQ ID NO:1195), CeresClone:341749 (SEQ ID NO:1196), CeresClone:666962 (SEQ ID NO:1197), CeresClone:522672 (SEQ ID NO:1198), gi|11602747 (SEQ ID NO:1199), and gi|11602749 (SEQ ID NO:1200). The consensus sequence determined by the alignment is set forth.

FIG. 117 is an alignment of the amino acid sequence of Lead cDNA ID 23785125 (SEQ ID NO:1202) with homologous and/or orthologous amino acid sequences CeresClone:841321 (SEQ ID NO:1203), gi|55773842 (SEQ ID NO:1204), CeresClone:601248 (SEQ ID NO:1205), gi|42794937 (SEQ ID NO:1206), CeresClone:959875 (SEQ ID NO:1207), and gi|28372932 (SEQ ID NO:1208). The consensus sequence determined by the alignment is set forth.

FIG. 118 is an alignment of the amino acid sequence of Lead cDNA ID 23699071 (SEQ ID NO:1212) with homologous and/or orthologous amino acid sequences CeresClone:643026 (SEQ ID NO:1213), gi|31430853 (SEQ ID NO:1214), CeresClone:329797 (SEQ ID NO:1215), CeresClone:38757 (SEQ ID NO:1216), gi|30681003 (SEQ ID NO:1217), and CeresClone:570295 (SEQ ID NO:1218). The consensus sequence determined by the alignment is set forth.

FIG. 119 is an alignment of the amino acid sequence of Lead cDNA ID 23527182 (SEQ ID NO:1220) with homologous and/or orthologous amino acid sequences CeresClone:1334990 (SEQ ID NO:1221), gi|20466045 (SEQ ID NO:1222), gi|12711287 (SEQ ID NO:1223), and CeresClone:473814 (SEQ ID NO:1224). The consensus sequence determined by the alignment is set forth.

FIG. 120 is an alignment of the amino acid sequence of Lead cDNA ID 23747378 (SEQ ID NO:1226) with homologous and/or orthologous amino acid sequences gi|62122347 (SEQ ID NO:1227), gi|5019464 (SEQ ID NO:1228), gi|51849631 (SEQ ID NO:1229), gi|51849641 (SEQ ID NO:1230), gi|51849637 (SEQ ID NO:1231), CeresClone:700266 (SEQ ID NO:1232), CeresClone:465896 (SEQ ID NO:1233), gi|37993053 (SEQ ID NO:1235), gi|34910770 (SEQ ID NO:1237), gi|51849651 (SEQ ID NO:1238), gi|51849635 (SEQ ID NO:1240), and gi|62867345 (SEQ ID NO:1241). The consensus sequence determined by the alignment is set forth.

FIG. 121 is an alignment of the amino acid sequence of Lead cDNA ID 23691708 (SEQ ID NO:1243) with homologous and/or orthologous amino acid sequences gi|9755785 (SEQ ID NO:1244), CeresClone:833439 (SEQ ID NO:1245), and gi|50911677 (SEQ ID NO:1246). The consensus sequence determined by the alignment is set forth.

FIG. 122 is an alignment of the amino acid sequence of Lead cDNA ID 23697027 (SEQ ID NO:1248) with homologous and/or orthologous amino acid sequences gi|23197970 (SEQ ID NO:1249), CeresClone:578919 (SEQ ID NO:1250), gi|34909052 (SEQ ID NO:1251), gi|50939567 (SEQ ID NO:1252), and CeresClone:504165 (SEQ ID NO:1253). The consensus sequence determined by the alignment is set forth.

FIG. 123 is an alignment of the amino acid sequence of Lead cDNA ID 23416843 (SEQ ID NO:1255) with homologous and/or orthologous amino acid sequences CeresClone:554630 (SEQ ID NO:1256), gi|50911677 (SEQ ID NO:1257), and CeresClone:833439 (SEQ ID NO:1259). The consensus sequence determined by the alignment is set forth.

FIG. 124 is an alignment of the amino acid sequence of Lead cDNA ID 23449314 (SEQ ID NO:1261) with homologous and/or orthologous amino acid sequences gi|56749359 (SEQ ID NO:1262), gi|13346194 (SEQ ID NO:1267), gi|39725415 (SEQ ID NO:1269), gi|31980095 (SEQ ID NO:1270), gi|1167484 (SEQ ID NO:1271), gi|50726662 (SEQ ID NO:1272), gi|19053 (SEQ ID NO:1273), CeresClone:1459729 (SEQ ID NO:1276), and gi|47680445 (SEQ ID NO:1277). The consensus sequence determined by the alignment is set forth.

FIG. 125 is an alignment of the amino acid sequence of Lead cDNA ID 23390282 (SEQ ID NO:1279) with homologous and/or orthologous amino acid sequences CeresClone:3244 (SEQ ID NO:1280), CeresClone:39985 (SEQ ID NO:1282), CeresClone:1020238 (SEQ ID NO:1287), CeresClone:18215 (SEQ ID NO:1288), CeresClone:111974 (SEQ ID NO:1290), CeresClone:207629 (SEQ ID NO:1291), gi|6979332 (SEQ ID NO:1293), gi|2437817 (SEQ ID NO:1294), and gi|100409 (SEQ ID NO:1295). The consensus sequence determined by the alignment is set forth.

FIG. 126 is an alignment of the amino acid sequence of Lead cDNA ID 23380202 (SEQ ID NO:1297) with homologous and/or orthologous amino acid sequences gi|55441974 (SEQ ID NO:1298), gi|49182274 (SEQ ID NO:1300), gi|49182280 (SEQ ID NO:1301), gi|21552981 (SEQ ID NO:1302), gi|60308938 (SEQ ID NO:1303), CeresClone:777105 (SEQ ID NO:1305), gi|33087075 (SEQ ID NO:1306), CeresClone:404146 (SEQ ID NO:1307), and gi|49182284 (SEQ ID NO:1308). The consensus sequence determined by the alignment is set forth.

FIG. 127 is an alignment of the amino acid sequence of Lead cDNA ID 23396143 (SEQ ID NO:1310) with homologous and/or orthologous amino acid sequences gi|50948537 (SEQ ID NO:1312), CeresClone:476283 (SEQ ID NO:1313), gi|7716952 (SEQ ID NO:1314), gi|21105746 (SEQ ID NO:1315), gi|40647397 (SEQ ID NO:1316), gi|34902994 (SEQ ID NO:1317), gi|14485513 (SEQ ID NO:1318), and CeresClone:461297 (SEQ ID NO:1319). The consensus sequence determined by the alignment is set forth.

FIG. 128 is an alignment of the amino acid sequence of Lead cDNA ID 23420963 (SEQ ID NO:1323) with homologous and/or orthologous amino acid sequences gi|38196019 (SEQ ID NO:1324), gi|38260618 (SEQ ID NO:1325), gi|38260631 (SEQ ID NO:1326), gi|9759579 (SEQ ID NO:1327), gi|38260685 (SEQ ID NO:1328), gi|34013890 (SEQ ID NO:1330), and gi|38260649 (SEQ ID NO:1331). The consensus sequence determined by the alignment is set forth.

FIG. 129 is an alignment of the amino acid sequence of Lead cDNA ID 23369680 (SEQ ID NO:1335) with homologous and/or orthologous amino acid sequences gi|34902106 (SEQ ID NO:1336), CeresClone:677852 (SEQ ID NO:1337), and CeresClone:637282 (SEQ ID NO:1338). The consensus sequence determined by the alignment is set forth.

FIG. 130 is an alignment of the amino acid sequence of Lead cDNA ID 23377150 (SEQ ID NO:1353) with homologous and/or orthologous amino acid sequences gi|30575840 (SEQ ID NO:1354), gi|22795039 (SEQ ID NO:1355), and CeresClone:543289 (SEQ ID NO:1356). The consensus sequence determined by the alignment is set forth.

FIG. 131 is an alignment of the amino acid sequence of Lead cDNA ID 23402435 (SEQ ID NO:1358) with homologous and/or orthologous amino acid sequences gi|33320073 (SEQ ID NO:1359) and gi|15810645 (SEQ ID NO:1360). The consensus sequence determined by the alignment is set forth.

FIG. 132 is an alignment of the amino acid sequence of Lead cDNA ID 23418435 (SEQ ID NO:1369) with homologous and/or orthologous amino acid sequences CeresClone:516050 (SEQ ID NO:1370) and CeresClone:775356 (SEQ ID NO:1371). The consensus sequence determined by the alignment is set forth.

FIG. 133 is an alignment of the amino acid sequence of Lead cDNA ID 23367406 (SEQ ID NO:1382) with homologous and/or orthologous amino acid sequences CeresClone:142681 (SEQ ID NO:1383), CeresClone:1063835 (SEQ ID NO:1384), CeresClone:1027529 (SEQ ID NO:1385), gi|21133 (SEQ ID NO:1386), gi|11133887 (SEQ ID NO:1387), CeresClone:1139782 (SEQ ID NO:1388), gi|42569485 (SEQ ID NO:1390), CeresClone:982579 (SEQ ID NO:1391), and gi|7443216 (SEQ ID NO:1392). The consensus sequence determined by the alignment is set forth.

FIG. 134 is an alignment of the amino acid sequence of Lead cDNA ID 23368554 (5110E2; SEQ ID NO:1394) with homologous and/or orthologous amino acid sequences CeresClone:221673 (SEQ ID NO:1395), gi|62733508 (SEQ ID NO:1396), CeresClone:633261 (SEQ ID NO:1397), and gi|14091850 (SEQ ID NO:1398). The consensus sequence determined by the alignment is set forth.

FIG. 135 is an alignment of the amino acid sequence of Lead cDNA ID 23368864 (5109H5; SEQ ID NO:1401) with homologous and/or orthologous amino acid sequence CeresClone:675752 (SEQ ID NO:1402). The consensus sequence determined by the alignment is set forth.

FIG. 136 is an alignment of the amino acid sequence of Lead cDNA ID 23372744 (SEQ ID NO:1404) with homologous and/or orthologous amino acid sequences gi|25518040 (SEQ ID NO:1405), CeresClone:971321 (SEQ ID NO:1406), CeresClone:529941 (SEQ ID NO:1407). CeresClone:390400 (SEQ ID NO:1408), CeresClone:237172 (SEQ ID NO:1409), CeresClone:1403244 (SEQ ID NO:1410), and CeresClone:516604 (SEQ ID NO:1411). The consensus sequence determined by the alignment is set forth.

FIG. 137 is an alignment of the amino acid sequence of Lead cDNA ID 23374628 (SEQ ID NO:1413) with homologous and/or orthologous amino acid sequences gi|15238624 (SEQ ID NO:1414), CeresClone:497385 (SEQ ID NO:1415), CeresClone:639274 (SEQ ID NO:1416), gi|50905733 (SEQ ID NO:1417), CeresClone:981348 (SEQ ID NO:1418), and CeresClone:812524 (SEQ ID NO:1419). The consensus sequence determined by the alignment is set forth.

FIG. 138 is an alignment of the amino acid sequence of Lead cDNA ID 23516818 (5109A1; SEQ ID NO:1423) with homologous and/or orthologous amino acid sequences gi|11249497 (SEQ ID NO:1424), gi|50940815 (SEQ ID NO:1425), gi|18481718 (SEQ ID NO:1426), and CeresClone:244116 (SEQ ID NO:1427). The consensus sequence determined by the alignment is set forth.

FIG. 139 is an alignment of the amino acid sequence of Lead cDNA ID 23699979 (SEQ ID NO:1429) with homologous and/or orthologous amino acid sequences gi|10177422 (SEQ ID NO:1430), gi|55296998 (SEQ ID NO:1436), CeresClone:238929 (SEQ ID NO:1437), and CeresClone:686876 (SEQ ID NO:1438). The consensus sequence determined by the alignment is set forth.

FIG. 140 is an alignment of the amino acid sequence of Lead cDNA ID 23814706 (SEQ ID NO:1440) with homologous and/or orthologous amino acid sequences CeresClone:1349 (SEQ ID NO:1441), CeresClone:1099781 (SEQ ID NO:1446), CeresClone:1066463 (SEQ ID NO:1447), CeresClone:476445 (SEQ ID NO:1448), CeresClone:327449 (SEQ ID NO:1449), and gi|37991859 (SEQ ID NO:1450). The consensus sequence determined by the alignment is set forth.

### DETAILED DESCRIPTION

Applicants have discovered novel methods of screening for regulatory proteins that can modulate expression of a gene, e.g., a reporter gene, operably linked to a regulatory region, such as a regulatory region involved in alkaloid biosynthesis. These discoveries can be used to create plant cells and plants containing (1) a nucleic acid encoding a regulatory protein, and/or (2) a nucleic acid including a regulatory region associated with a given regulatory protein, e.g., to modulate expression of a sequence of interest operably linked to the regulatory region.

Thus, in one aspect, this document relates to a method for identifying a regulatory protein capable of activating a regulatory region. The method involves screening for the ability of the regulatory protein to modulate expression of a reporter that is operably linked to the regulatory region. The ability of the regulatory protein to modulate expression of the reporter is determined by monitoring reporter activity.

A regulatory protein and a regulatory region are considered to be “associated” when the regulatory protein is capable of modulating expression, either directly or indirectly, of a nucleic acid operably linked to the regulatory region. For example, a regulatory protein and a regulatory region can be said to be associated when the regulatory protein directly binds to the regulatory region, as in a transcription factor-promoter complex. In other cases, a regulatory protein and regulatory region can be said to be associated when the regulatory protein does not directly bind to the regulatory region. A regulatory protein and a regulatory region can also be said to be associated when the regulatory protein indirectly affects transcription by being a component of a protein complex involved in transcriptional regulation or by noncovalently binding to a protein complex involved in transcriptional regulation. In some cases, a regulatory protein and regulatory region can be said to be associated and indirectly affect transcription when the regulatory protein participates in or is a component of a signal transduction cascade or a proteasome degradation pathway, e.g., of repressors, that results in transcriptional amplification or repression. In some cases, regulatory proteins associate with regulatory regions and indirectly affect transcription by, e.g., binding to methylated DNA, unwinding chromatin, binding to RNA, or modulating splicing.

A regulatory protein and its associated regulatory region can be used to selectively modulate expression of a sequence of interest, when such a sequence is operably linked to the regulatory region. In addition, the use of such regulatory protein-regulatory region associations in plants can permit selective modulation of the amount or rate of biosynthesis of plant polypeptides and plant compounds, such as alkaloid compounds, under a desired environmental condition or in a desired plant developmental pathway. For example, the use of recombinant regulatory proteins in plants, such as Papaveraceae plants, that are capable of producing one or more alkaloids, can permit selective modulation of the amount of such compounds in such plants.

### Polypeptides

The term “polypeptide” as used herein refers to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. The term “amino acid” refers to natural and/or unnatural or synthetic amino acids, including D/L optical isomers. Full-length proteins, analogs, mutants, and fragments thereof are encompassed by this definition.

The term “isolated” with respect to a polypeptide refers to a polypeptide that has been separated from cellular components that naturally accompany it. Typically, the polypeptide is isolated when it is at least 60%, e.g., 70%, 80%, 90%, 95%, or 99%, by weight, free from proteins and naturally occurring organic molecules that are naturally associated with it. In general, an isolated polypeptide will yield a single major band on a reducing and/or non-reducing polyacrylamide gel. Isolated polypeptides can be obtained, for example, by extraction from a natural source (e.g., plant tissue), chemical synthesis, or by recombinant production in a host plant cell. To recombinantly produce a polypeptide, a nucleic acid sequence containing a nucleotide sequence encoding a polypeptide of interest can be ligated into an expression vector and used to transform a bacterial, eukaryotic, or plant host cell, e.g., insect, yeast, mammalian, or plant cells.

Polypeptides described herein include regulatory proteins. Such a regulatory protein typically is effective for modulating expression of a nucleic acid sequence operably linked to a regulatory region involved in an alkaloid biosynthesis pathway, such as a nucleic acid sequence encoding a polypeptide involved in alkaloid biosynthesis. Modulation of expression of a nucleic acid sequence can be either an increase or a decrease in expression of the nucleic acid sequence relative to the average rate or level of expression of the nucleic acid sequence in a control plant.

A regulatory protein can have one or more domains characteristic of a zinc finger transcription factor polypeptide. For example, a regulatory protein can contain a zf-C3HC4 domain characteristic of a C3HC4 type (RING finger) zinc-finger polypeptide. The RING finger is a specialized type of zinc-finger of 40 to 60 residues that binds two atoms of zinc and is reported to be involved in mediating protein-protein interactions. There are two different variants, the C3HC4-type and a C3H2C3-type, which are related despite the different cysteine/histidine pattern. The RING domain has been implicated in diverse biological processes. Ubiquitin-protein ligases (E3s), which determine the substrate specificity for ubiquitylation, have been classified into HECT and RING-finger families. Various RING fingers exhibit binding to E2 ubiquitin-conjugating enzymes. SEQ ID NO:115, SEQ ID NO:168, SEQ ID NO:434, SEQ ID NO:492, SEQ ID NO:506, SEQ ID NO:608, SEQ ID NO:695, SEQ ID NO:1119, SEQ ID NO:1243, SEQ ID NO:1255, and SEQ ID NO:1335 set forth the amino acid sequences of DNA clones, identified herein as cDNA ID 23663607 (SEQ ID NO:114), cDNA ID 23547976 (SEQ ID NO:167), cDNA ID 23389418 (SEQ ID NO:433), cDNA ID 23500965 (SEQ ID NO:491), cDNA ID 24373996 (SEQ ID NO:505), cDNA ID 23529931 (SEQ ID NO:607), cDNA ID 23503210 (SEQ ID NO:694), cDNA ID 23389186 (SEQ ID NO:1118), cDNA ID 23691708 (SEQ ID NO:1242), cDNA ID 23416843 (SEQ ID NO:1254), and cDNA ID 23369680 (SEQ ID NO:1334), respectively, each of which is predicted to encode a C3HC4 type (RING finger) zinc-finger polypeptide.

In some cases, a regulatory protein can contain a zf-C3HC4 domain and a PA (protease associated) domain. A PA domain is found as an insert domain in diverse proteases, including the MEROPS peptidase families A22B, M28, and S8A. A PA domain is also found in a plant vacuolar sorting receptor and members of the RZF family. It has been suggested that this domain forms a lid-like structure that covers the active site in active proteases and is involved in protein recognition in vacuolar sorting receptors. SEQ ID NO:766 sets forth the amino acid sequence of a DNA clone, identified herein as cDNA ID 23772039 (SEQ ID NO:765), that is predicted to encode a polypeptide having a zf-C3HC4 domain and a PA domain.

In some cases, a regulatory protein can contain a zf-CCCH domain characteristic of C-x8-C-x5-C-x3-H type (and similar) zinc finger transcription factor polypeptides. Polypeptides containing zinc finger domains of the C-x8-C-x5-C-x3-H type include zinc finger polypeptides from eukaryotes involved in cell cycle or growth phase-related regulation, e.g. human TIS11B (butyrate response factor 1), a predicted regulatory protein involved in regulating the response to growth factors. Another protein containing this domain is the human splicing factor U2AF 35 kD subunit, which plays a critical role in both constitutive and enhancer-dependent splicing by mediating essential protein-protein interactions and protein-RNA interactions required for 3′ splice site selection. It has been shown that different CCCH zinc finger proteins interact with the 3′ untranslated regions of various mRNAs. SEQ ID NO:260, SEQ ID NO:368, and SEQ ID NO:458 set forth the amino acid sequences of DNA clones, identified herein as cDNA ID 23370190 (SEQ ID NO:259), cDNA ID 23692994 (SEQ ID NO:367), and cDNA ID 23365920 (SEQ ID NO:457), respectively, that are predicted to encode C-x8-C-x5-C-x3-H type zinc finger polypeptides.

In some cases, a regulatory protein having a zf-CCCH domain can also have an RNA recognition motif RNA recognition motifs, also known as RRM, RBD, or RNP domains, are found in a variety of RNA binding polypeptides, including heterogeneous nuclear ribonucleoproteins (hnRNPs), polypeptides implicated in regulation of alternative splicing, and polypeptide components of small nuclear ribonucleoproteins (snRNPs). The RRM motif also appears in a few single stranded DNA binding proteins. The RRM structure consists of four strands and two helices arranged in an alpha/beta sandwich, with a third helix present during RNA binding in some cases. SEQ ID NO:141 sets forth the amino acid sequence of a DNA clone, identified herein as cDNA ID 23447462 (SEQ ID NO:140), that is predicted to encode a polypeptide containing a zf-CCCH domain and an RRM_{—}1 domain.

In some cases, a regulatory protein having a zf-CCCH domain can also have a KH domain. The K homology (KH) domain is a widespread RNA-binding motif that has been detected by sequence similarity searches in such proteins as heterogeneous nuclear ribonucleoprotein K (hnRNP K) and ribosomal protein S3. Analysis of spatial structures of KH domains in hnRNP K and S3 has revealed that they are topologically dissimilar. The KH domain with a C-terminal βα extension has been named KH type I, and the KH domain with an N-terminal αβ extension has been named KH type II. KH motifs consist of about 70 amino acids. SEQ ID NO:1369 sets forth the amino acid sequence of a DNA clone, identified herein as cDNA ID 23418435 (SEQ ID NO:1368), that is predicted to encode a polypeptide containing a zf-CCCH domain and a KH domain.

In some cases, a regulatory protein can contain a zf-CCHC domain characteristic of a zinc knuckle polypeptide. The zinc knuckle is a zinc binding motif with the sequence CX2CX4HX4C, where X can be any amino acid. The motifs are common to the nucleocapsid proteins of retroviruses, and the prototype structure is from HIV. The zinc knuckle family also contains members involved in eukaryotic gene regulation. A zinc knuckle is found in eukaryotic proteins involved in RNA binding or single strand DNA binding. SEQ ID NO:229 and SEQ ID NO:657 set forth the amino acid sequences of DNA clones, identified herein as cDNA ID 13579142 (SEQ ID NO:228) and cDNA ID 23528916 (SEQ ID NO:656), respectively, each of which is predicted to encode a polypeptide having a zf-CCHC domain.

In some cases, a regulatory protein containing a zf-CCHC domain can also contain an RRM_{—}1 domain described above. SEQ ID NO:599 and SEQ ID NO:1171 set forth the amino acid sequences of DNA clones, identified herein as cDNA ID 23498294 (SEQ ID NO:598) and cDNA ID 23376628 (SEQ ID NO:1170), respectively, each of which is predicted to encode a polypeptide containing a zf-CCHC domain and an RRM_{—}1 domain.

In some cases, a regulatory protein can contain a zf-AN1 domain characteristic of an AN1-like zinc finger transcription factor polypeptide. The zf-AN1 domain was first identified as a zinc finger at the C-terminus of An1, a ubiquitin-like protein in Xenopus laevis. The following pattern describes the zinc finger: C—X2-C—X(9-12)-C—X(1-2)-C—X4-C—X2-H—X5-H—X—C, where X can be any amino acid, and the numbers in brackets indicate the number of residues. A zf-AN1 domain has been identified in a number of as yet uncharacterized proteins from various sources. SEQ ID NO:281 sets forth the amino acid sequence of a DNA clone, identified herein as cDNA ID 23364997 (SEQ ID NO:280), that is predicted to encode a zinc finger transcription factor polypeptide having a zf-AN1 domain.

In some cases, a regulatory protein having a zf-AN1 domain can also have a zf-A20 domain. A20 (an inhibitor of cell death)-like zinc fingers are believed to mediate self-association in A20. These fingers also mediate IL-1-induced NF-kappa B activation. SEQ ID NO:494 sets forth the amino acid sequence of a DNA clone, referred to herein as cDNA ID 23538950 (SEQ ID NO:493) that is predicted to encode a zinc finger transcription factor polypeptide having a zf-AN1 domain and a zf-A20 domain.

In some cases, a regulatory protein can contain one or more zf-C2H2 domains characteristic of C2H2 type zinc finger transcription factor polypeptides. C2H2 zinc-finger family polypeptides play important roles in plant development including floral organogenesis, leaf initiation, lateral shoot initiation, gametogenesis, and seed development. SEQ ID NO:716 sets forth the amino acid sequence of a DNA clone, identified herein as cDNA ID 23421865 (SEQ ID NO:715), that is predicted to encode a polypeptide containing a zf-C2H2 domain. SEQ ID NO:619 sets forth the amino acid sequence of a DNA clone, identified herein as cDNA ID 23515088 (SEQ ID NO:618) that is predicted to encode a C2H2 zinc-finger polypeptide containing two zf-C2H2 domains.

In some cases, a regulatory protein can contain a zf-B_box domain characteristic of a B-box zinc finger polypeptide. The B-box zinc finger domain consists of about 40 amino acids. One or two copies of the B-box domain are generally associated with a ring finger and a coiled coil motif to form the so-called tripartite motif. The B-box domain is found in transcription factors, ribonucleoproteins, and proto-oncoproteins. NMR analysis has revealed that the B-box structure comprises two beta-strands, two helical turns, and three extended loop regions different from any other zinc binding motif. SEQ ID NO:613 sets forth the amino acid sequence of a DNA clone, referred to herein as cDNA ID 23498685 (SEQ ID NO:612), that is predicted to encode a polypeptide containing a zf-B_box.

In some cases, a regulatory protein can contain a zf-D of domain characteristic of a D of domain zinc finger transcription factor polypeptide. D of (DNA binding with one finger) domain polypeptides are plant-specific transcription factor polypeptides having a highly conserved DNA binding domain. A D of domain is a zinc finger DNA binding domain that resembles the Cys2 zinc finger, although it has a longer putative loop containing an extra Cys residue that is conserved. AOBP, a DNA binding protein in pumpkin (Cucurbita maxima), contains a 52 amino acid D of domain, which is highly conserved in several DNA binding proteins of higher plants. SEQ ID NO:235 sets forth the amino acid sequence of a DNA clone, identified herein as cDNA ID 23365150 (SEQ ID NO:234) that is predicted to encode a D of domain zinc finger transcription factor polypeptide.

A regulatory protein can comprise the amino acid sequence set forth in SEQ ID NO:115, SEQ ID NO:168, SEQ ID NO:434, SEQ ID NO:492, SEQ ID NO:506, SEQ ID NO:608, SEQ ID NO:695, SEQ ID NO:1119, SEQ ID NO:1243, SEQ ID NO:1255, SEQ ID NO:1335, SEQ ID NO:766, SEQ ID NO:260, SEQ ID NO:368, SEQ ID NO:458, SEQ ID NO:141, SEQ ID NO:1369, SEQ ID NO:229, SEQ ID NO:657, SEQ ID NO:599, SEQ ID NO:1171, SEQ ID NO:281, SEQ ID NO:494, SEQ ID NO:716, SEQ ID NO:619, SEQ ID NO:613, or SEQ ID NO:235. Alternatively, a regulatory protein can be a homolog, ortholog, or variant of the polypeptide having the amino acid sequence set forth in SEQ ID NO:115, SEQ ID NO:168, SEQ ID NO:434, SEQ ID NO:492, SEQ ID NO:506, SEQ ID NO:608, SEQ ID NO:695, SEQ ID NO:1119, SEQ ID NO:1243, SEQ ID NO:1255, SEQ ID NO:1335, SEQ ID NO:766, SEQ ID NO:260, SEQ ID NO:368, SEQ ID NO:458, SEQ ID NO:141, SEQ ID NO:1369, SEQ ID NO:229, SEQ ID NO:657, SEQ ID NO:599, SEQ ID NO:1171, SEQ ID NO:281, SEQ ID NO:494, SEQ ID NO:716, SEQ ID NO:619, SEQ ID NO:613, or SEQ ID NO:235. For example, a regulatory protein can have an amino acid sequence with at least 30% sequence identity, e.g., 31%, 35%, 40%, 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:115, SEQ ID NO:168, SEQ ID NO:434, SEQ ID NO:492, SEQ ID NO:506, SEQ ID NO:608, SEQ ID NO:695, SEQ ID NO:1119, SEQ ID NO:1243, SEQ ID NO:1255, SEQ ID NO:1335, SEQ ID NO:766, SEQ ID NO:260, SEQ ID NO:368, SEQ ID NO:458, SEQ ID NO:141, SEQ ID NO:1369, SEQ ID NO:229, SEQ ID NO:657, SEQ ID NO:599, SEQ ID NO:1171, SEQ ID NO:281, SEQ ID NO:494, SEQ ID NO:716, SEQ ID NO:619, SEQ ID NO:613, or SEQ ID NO:235.

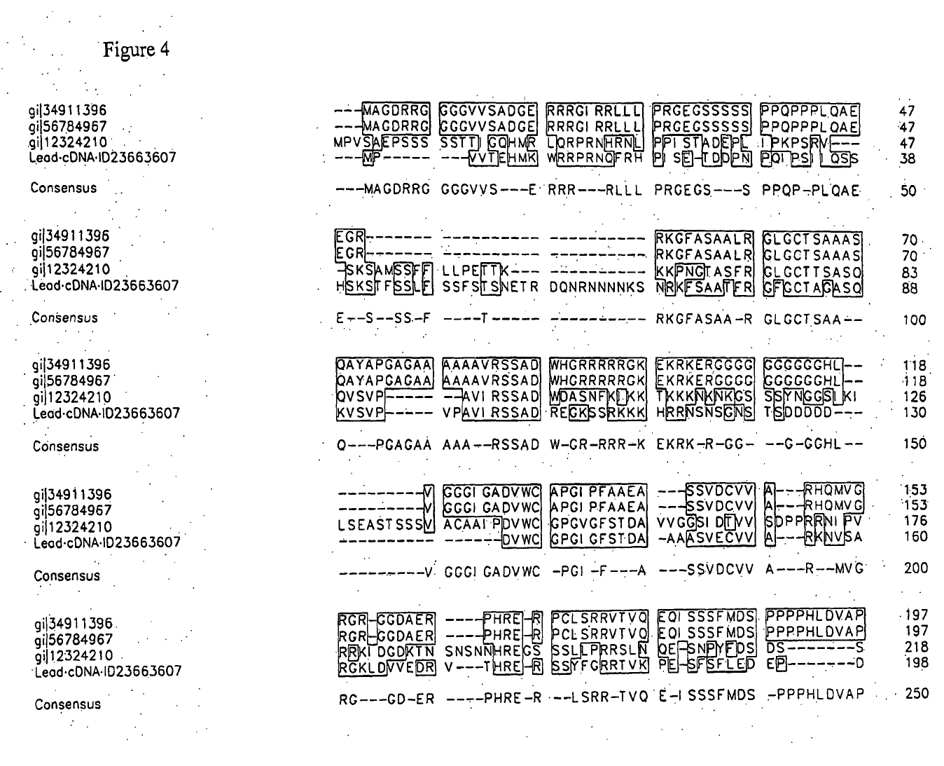

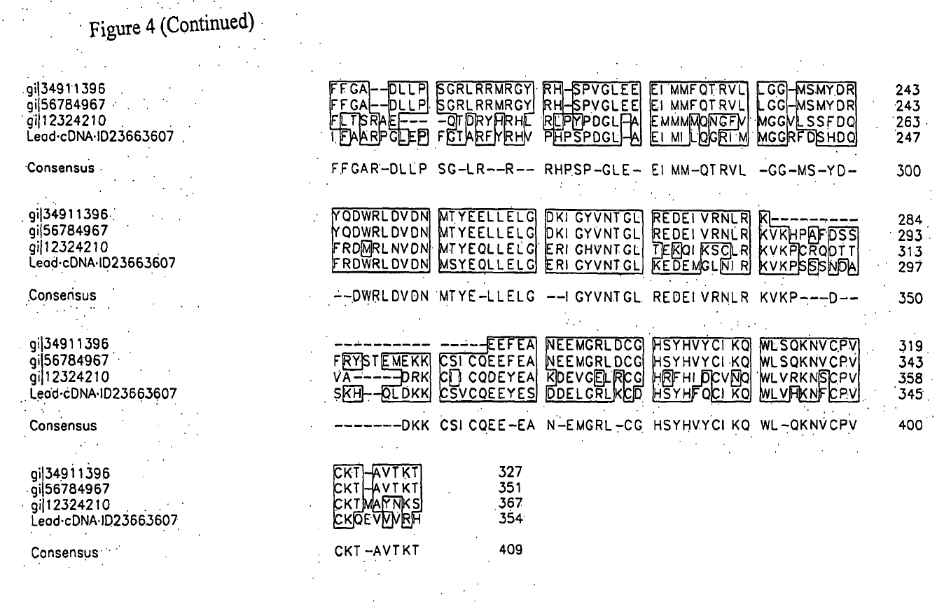

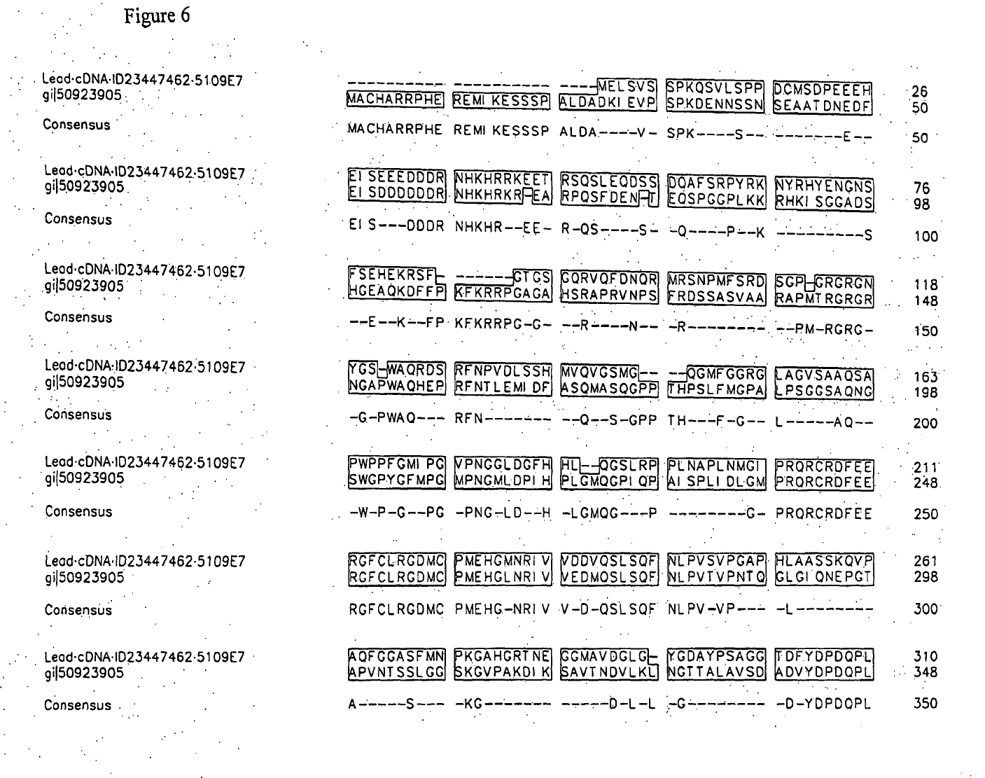

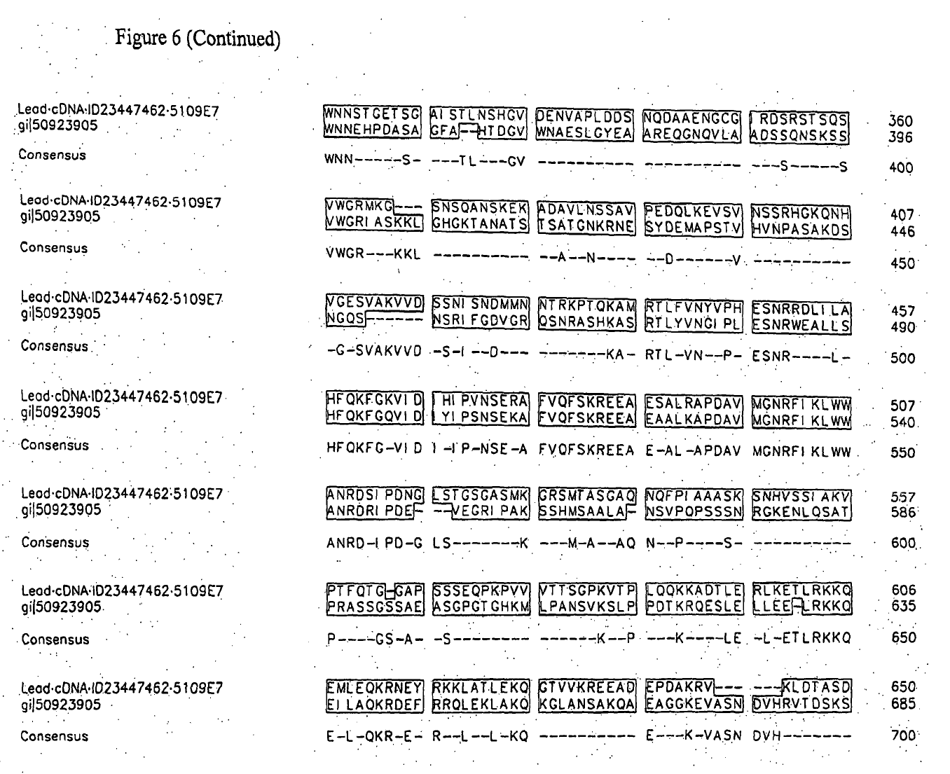

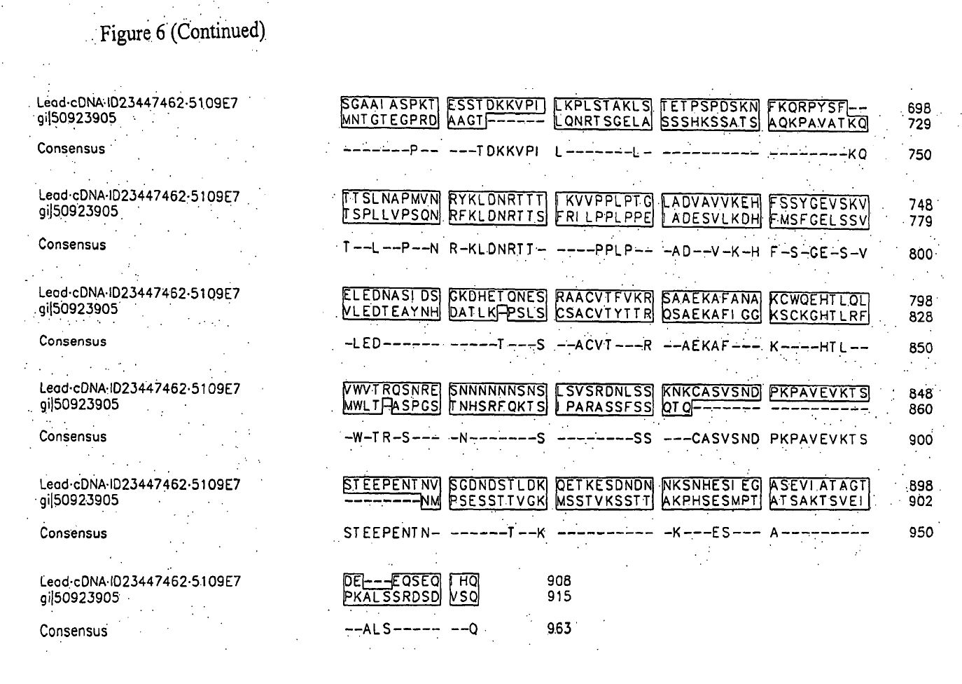

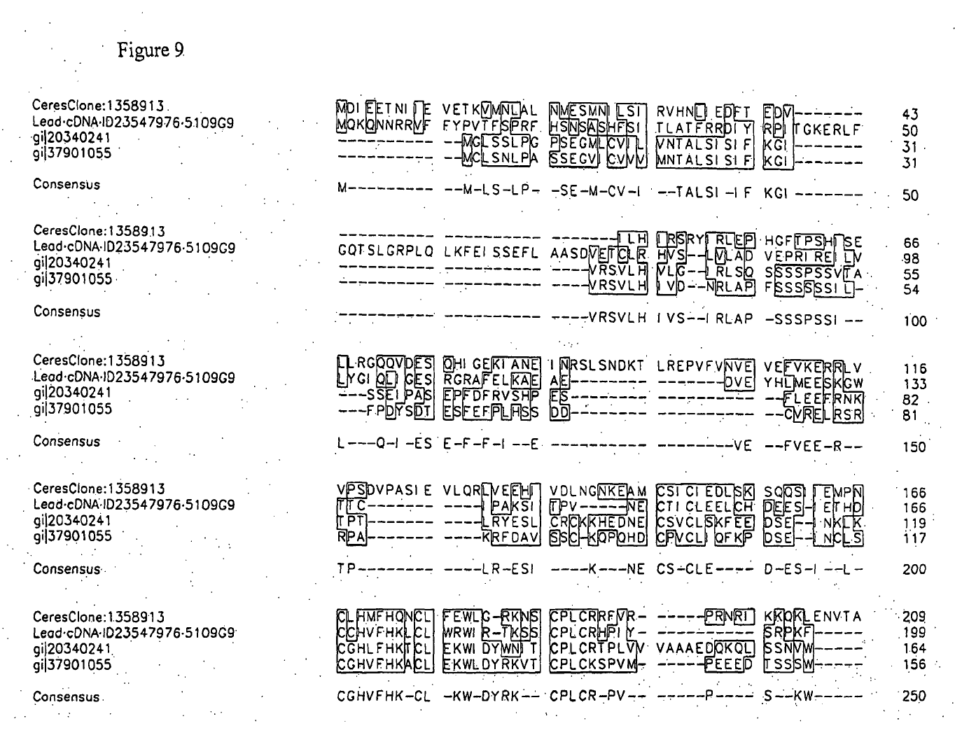

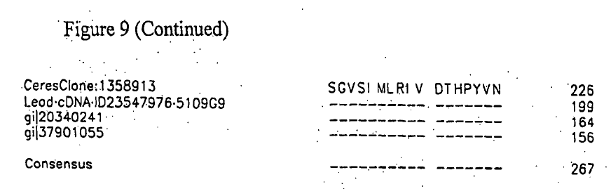

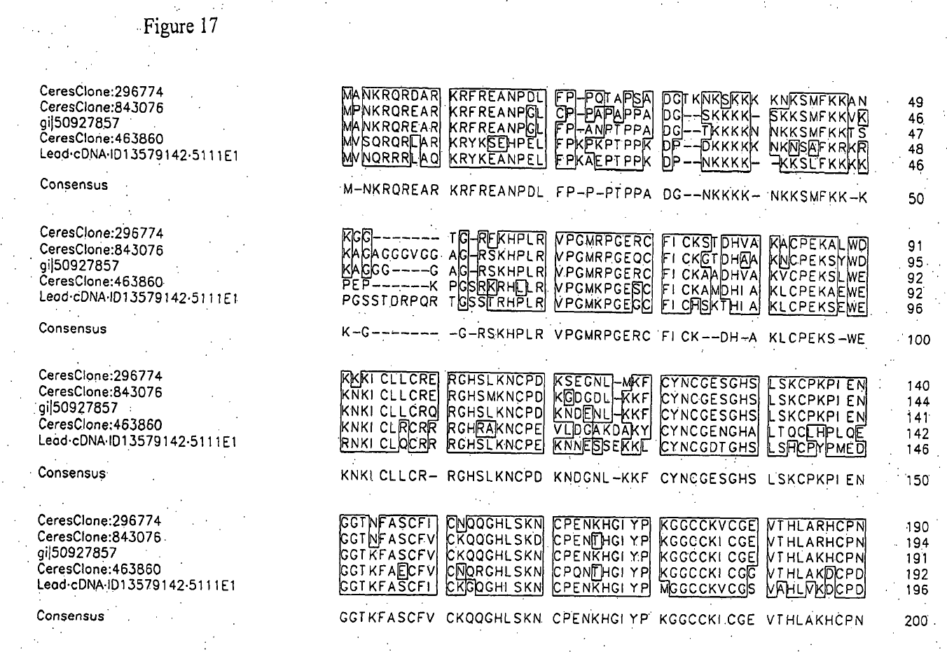

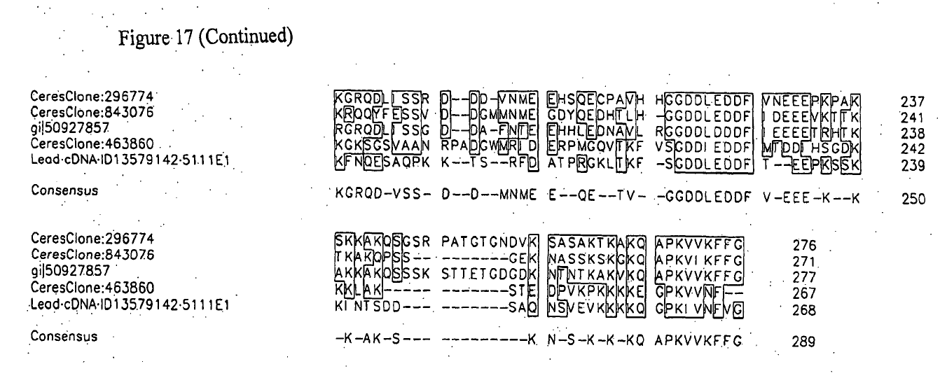

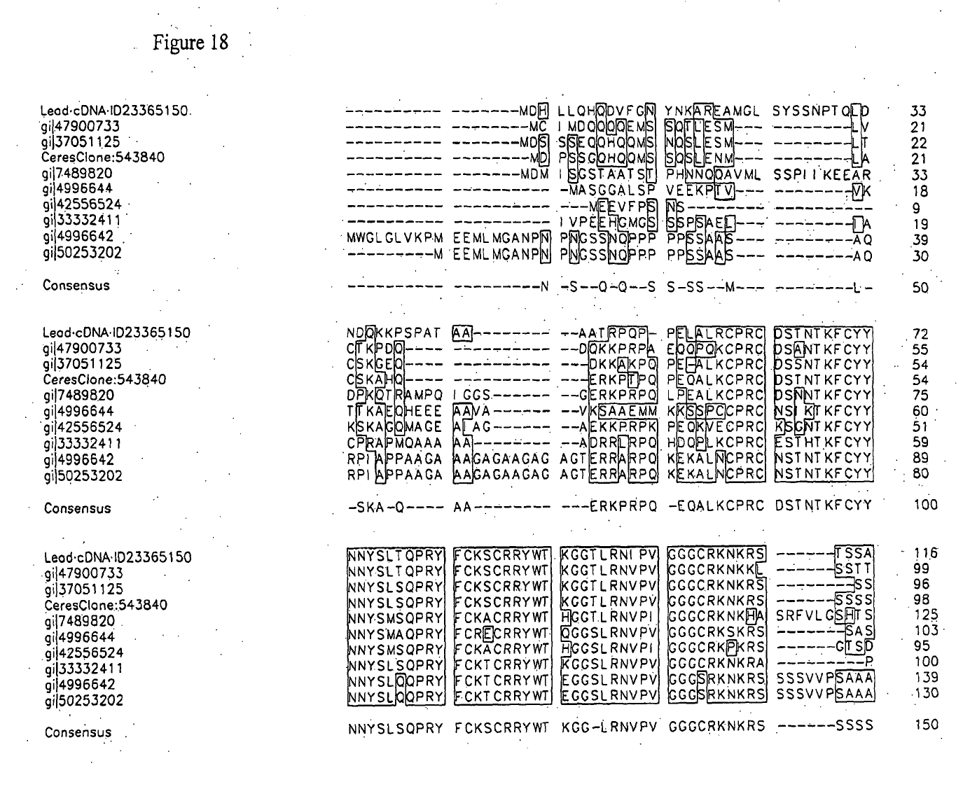

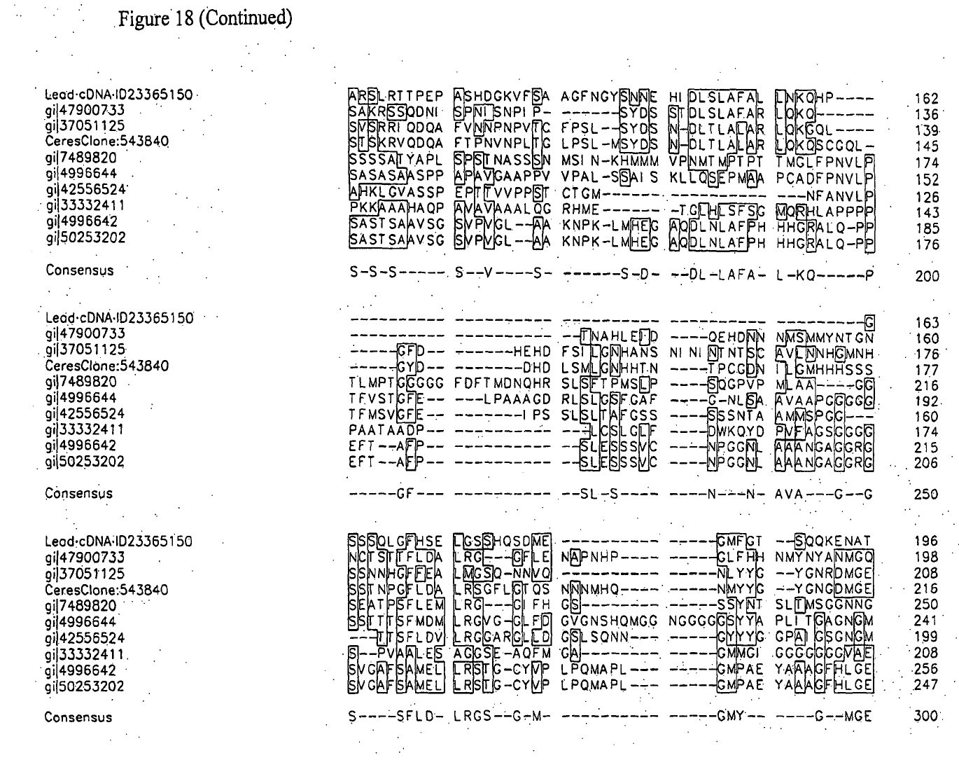

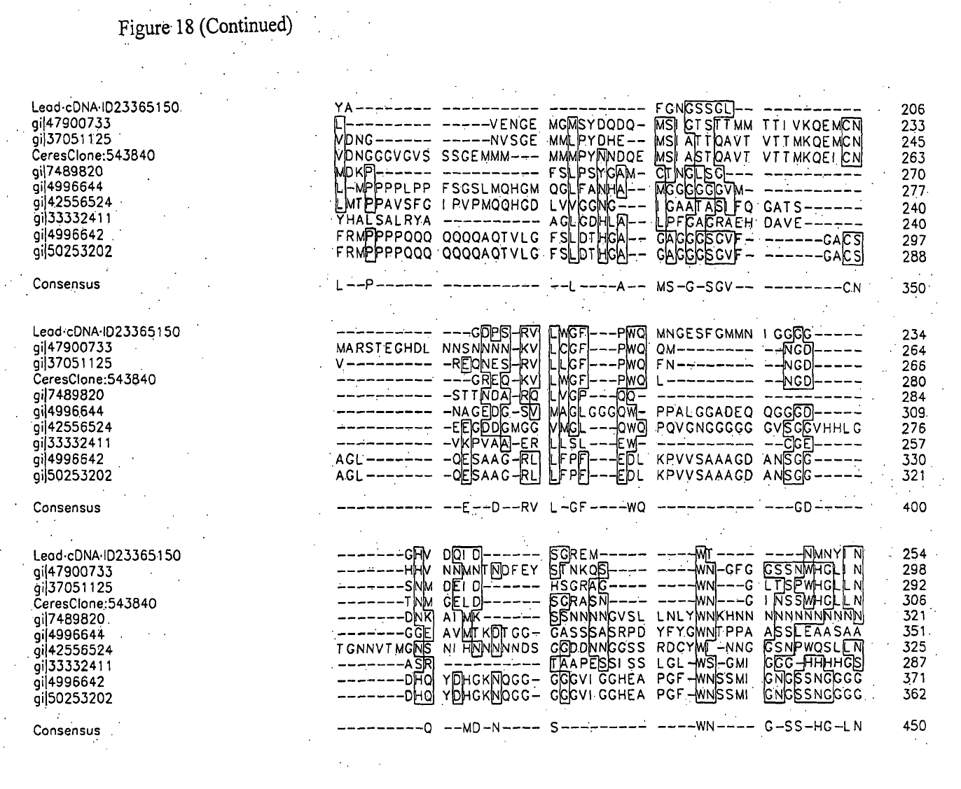

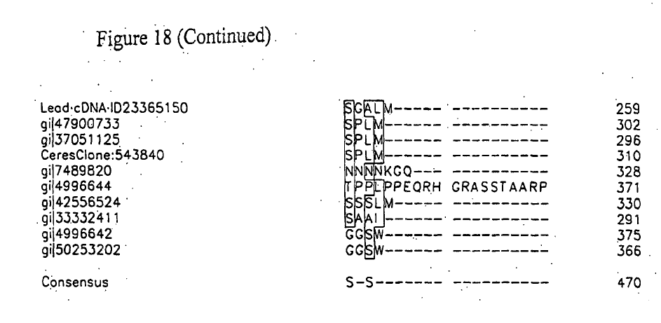

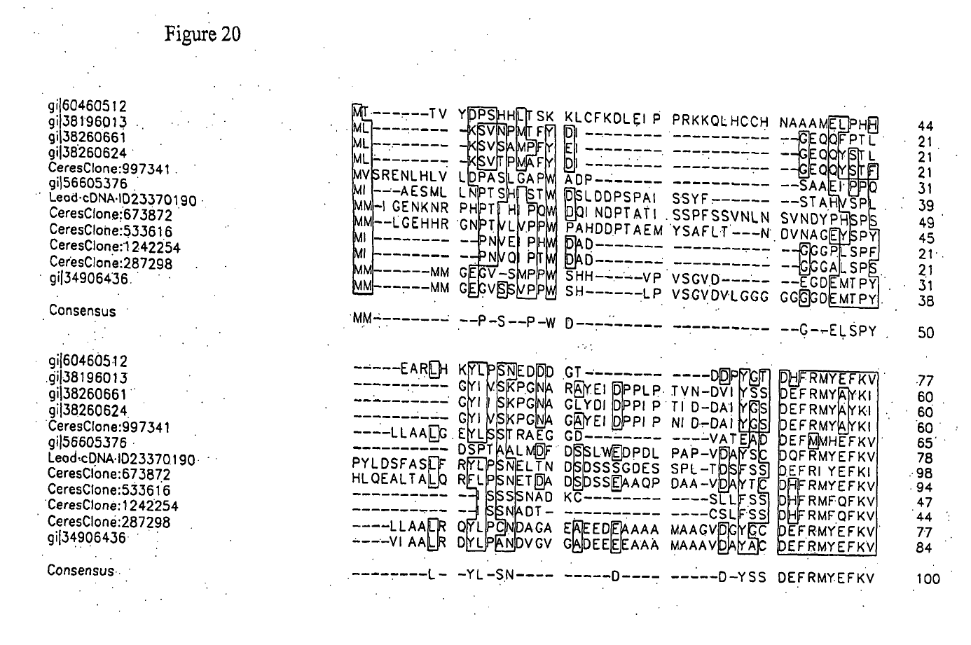

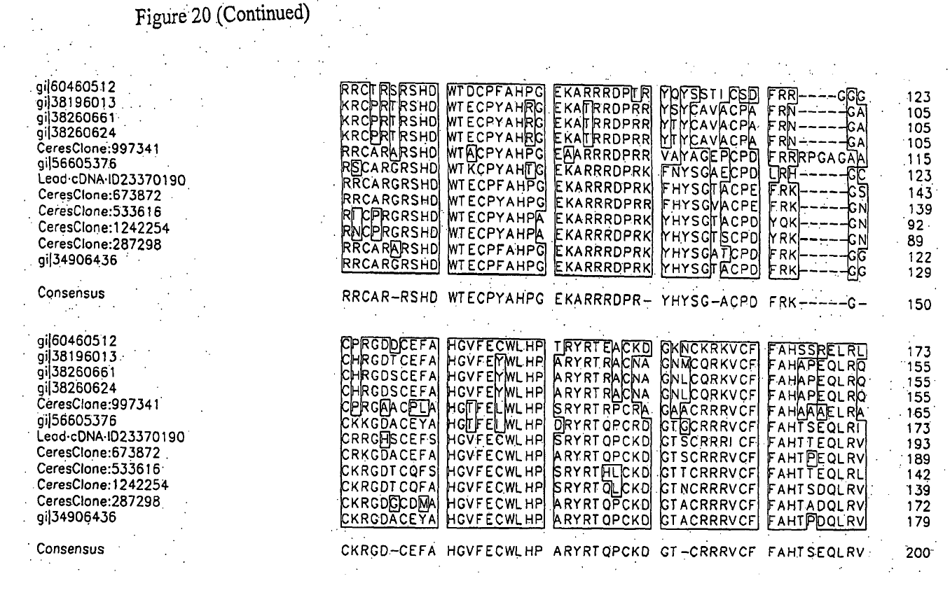

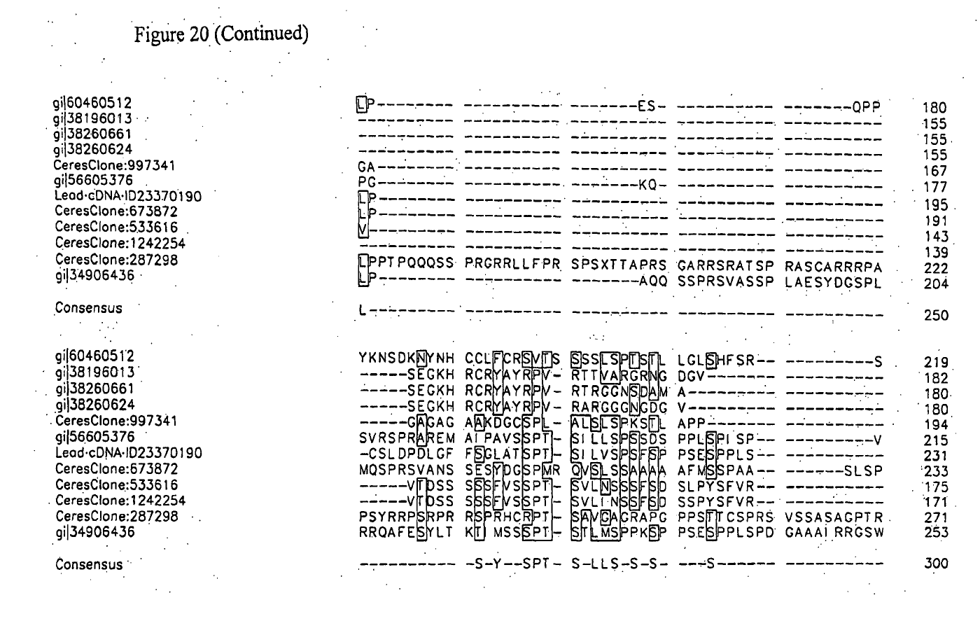

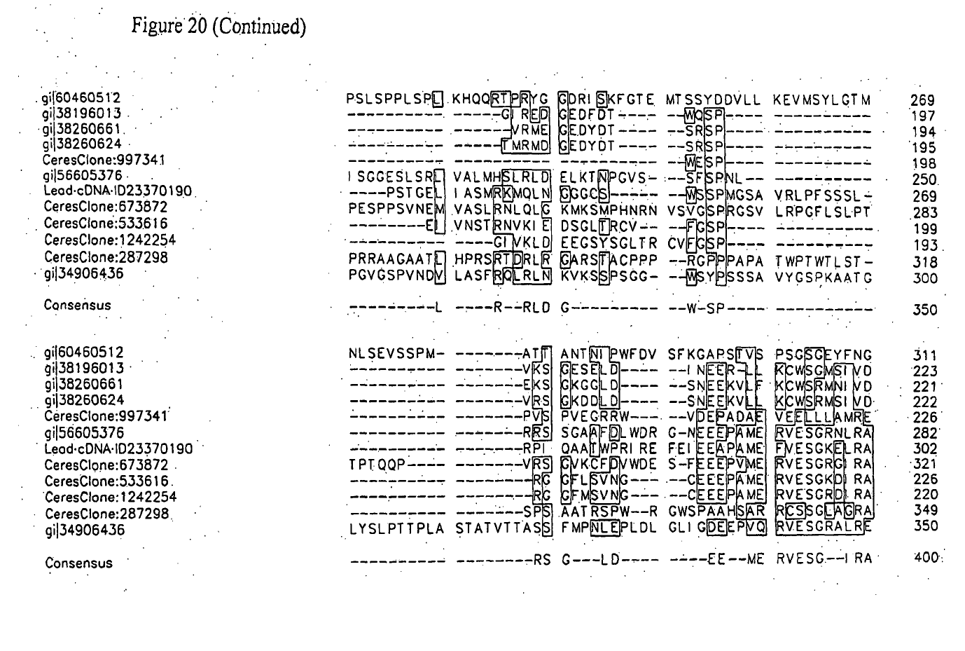

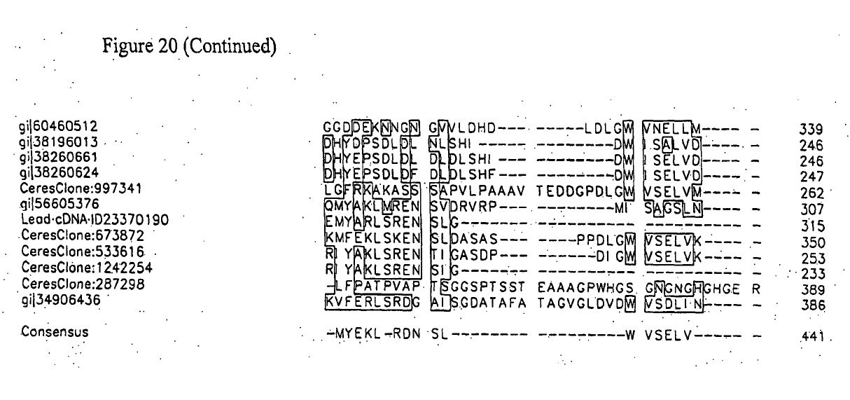

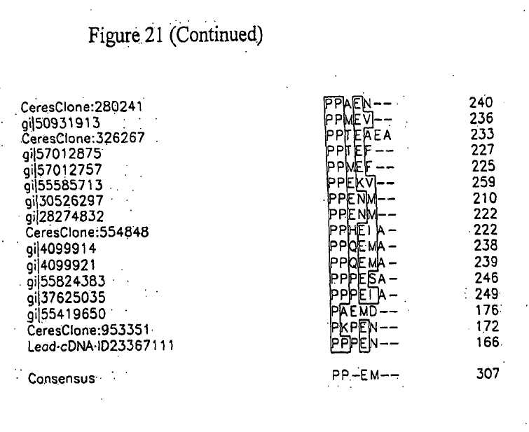

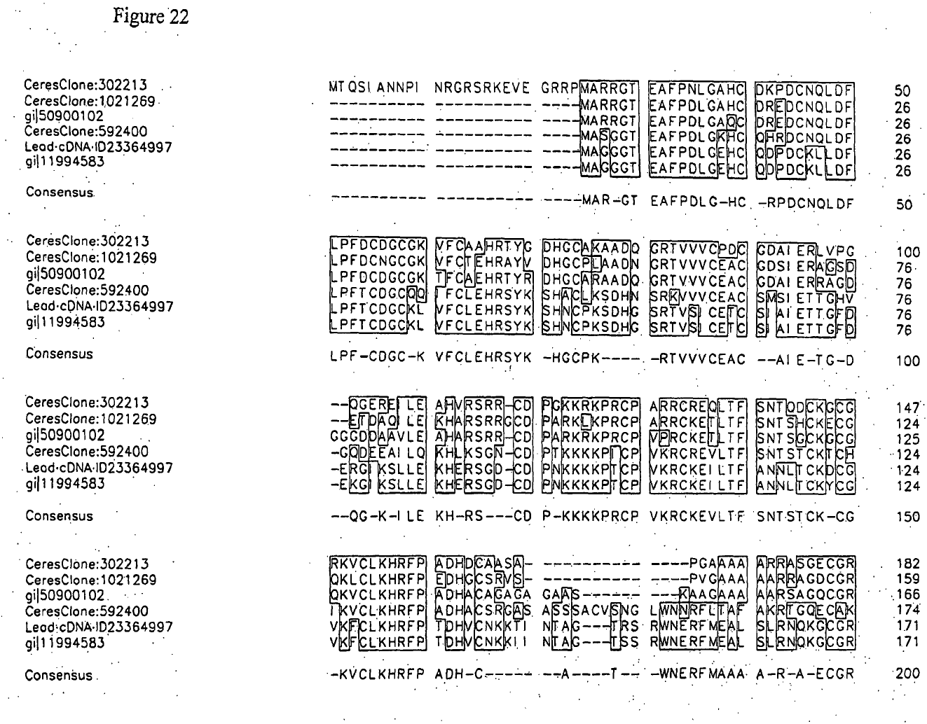

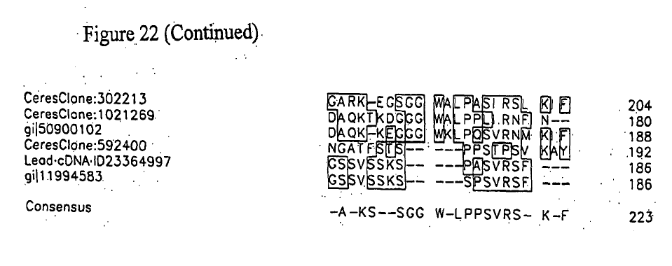

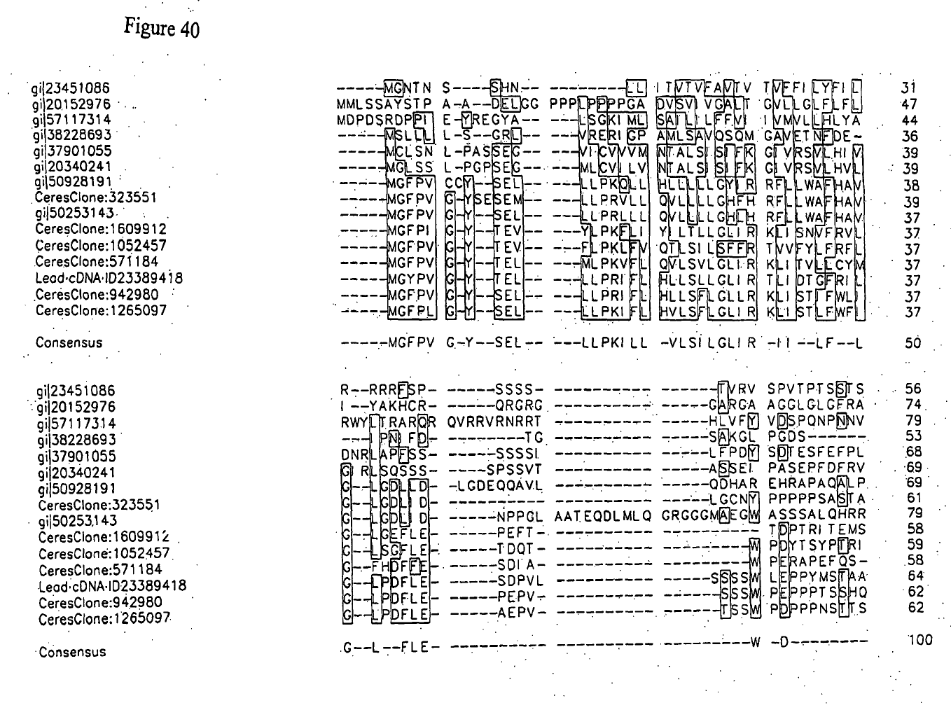

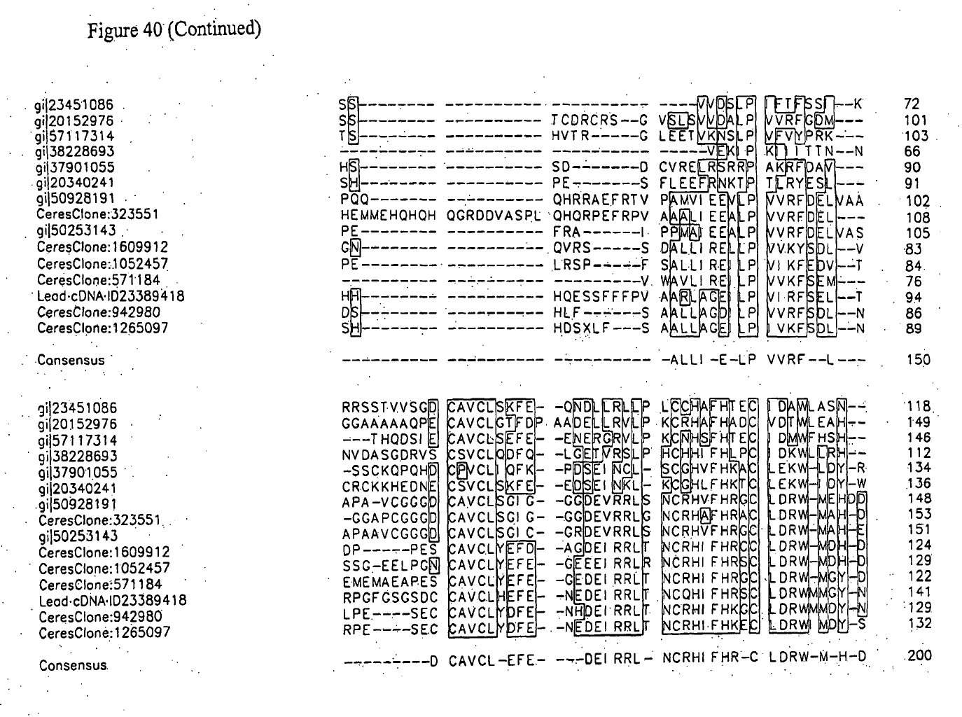

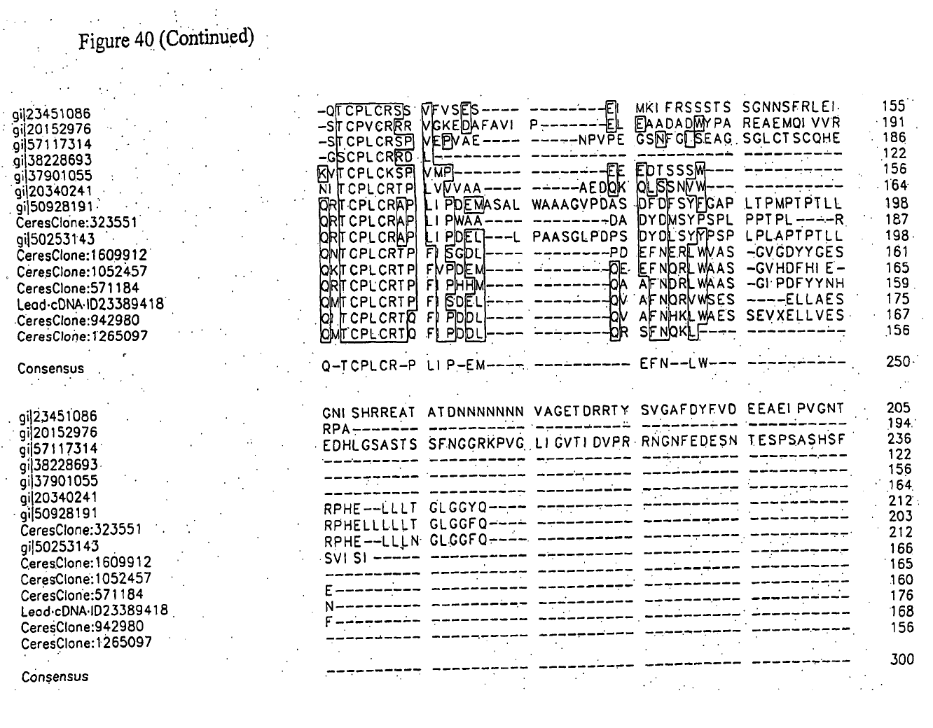

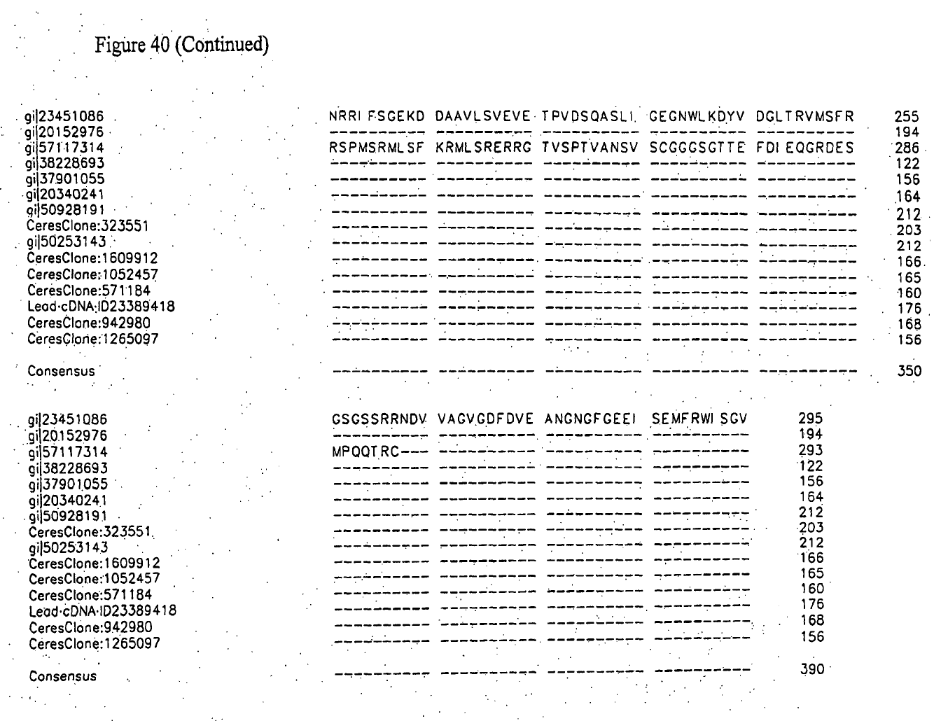

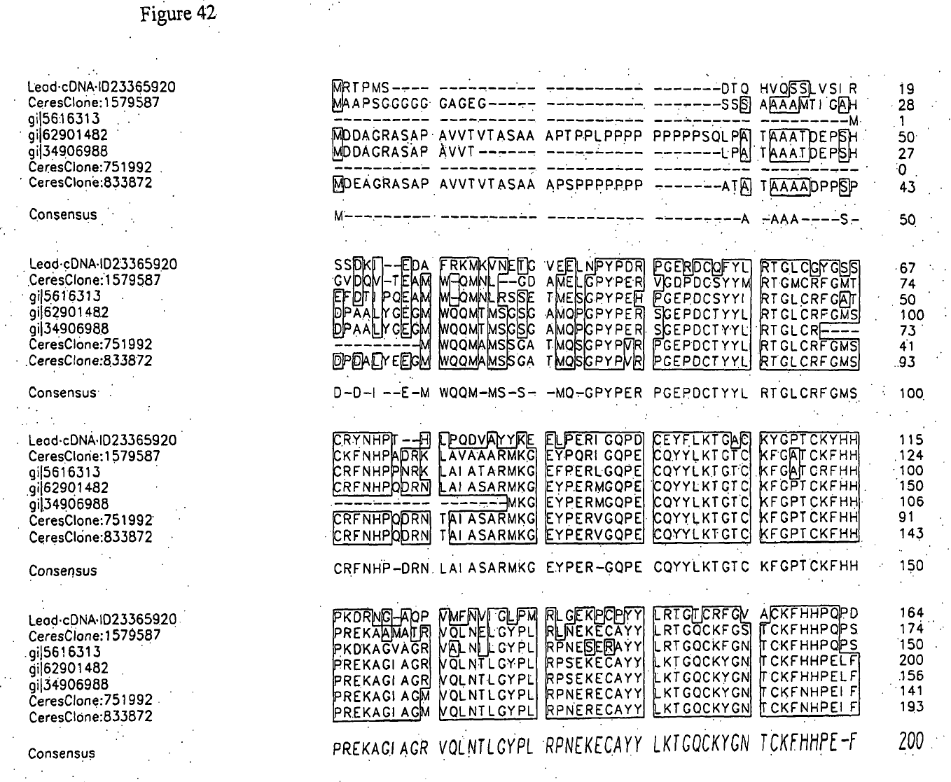

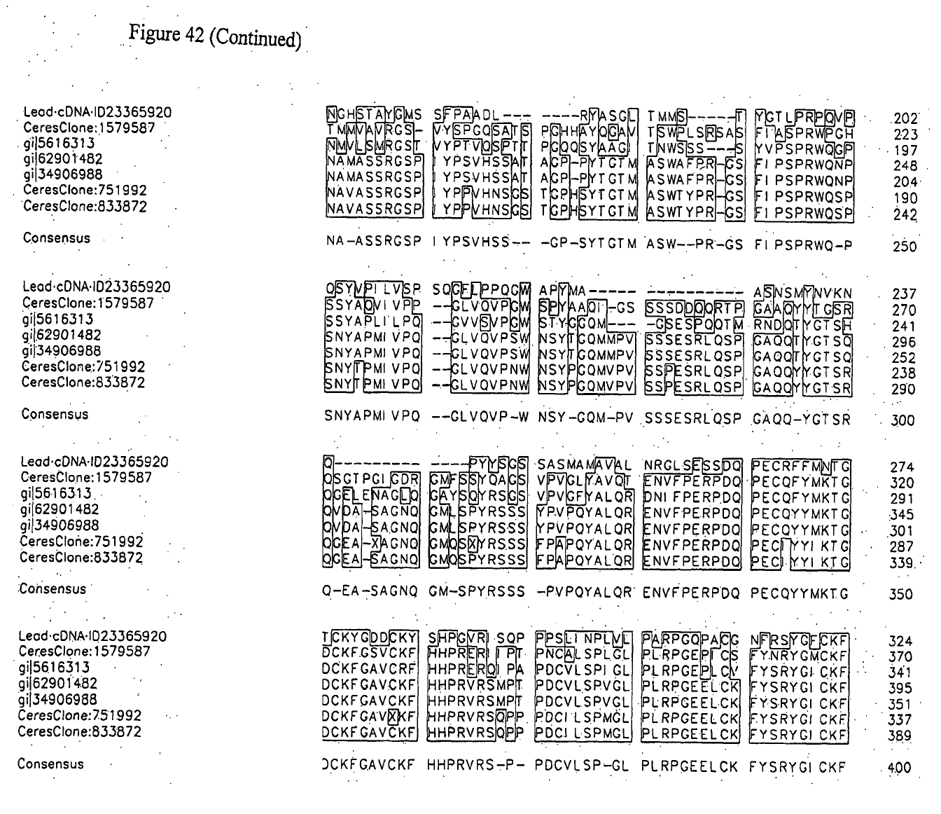

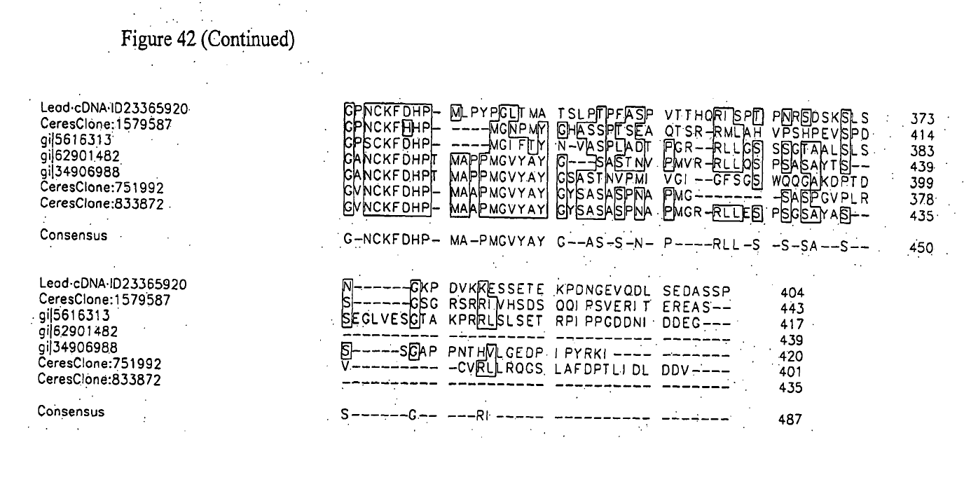

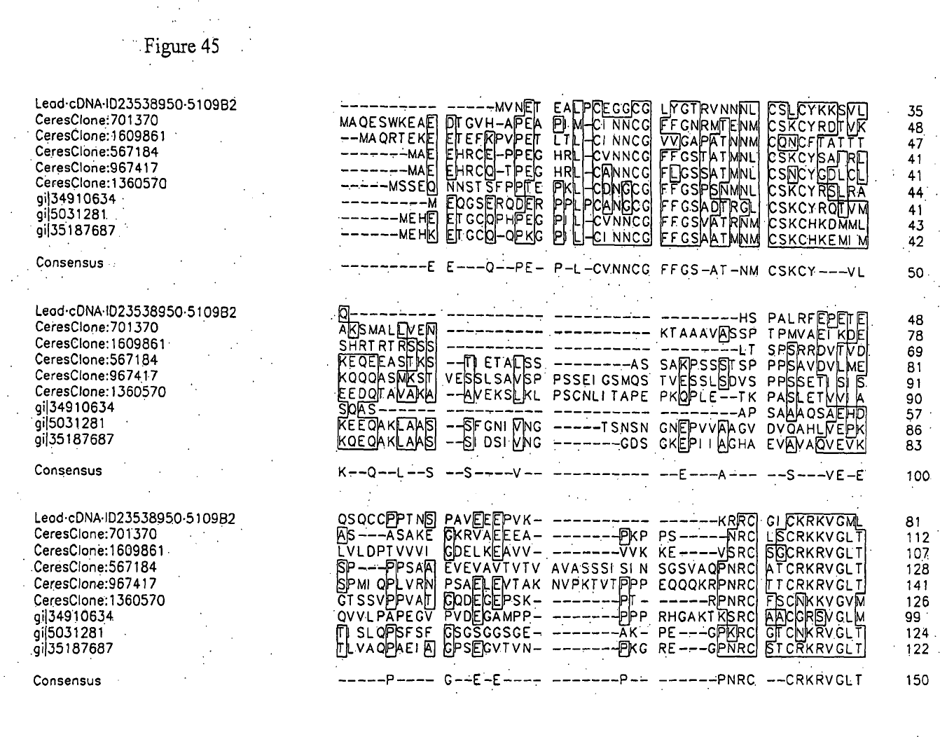

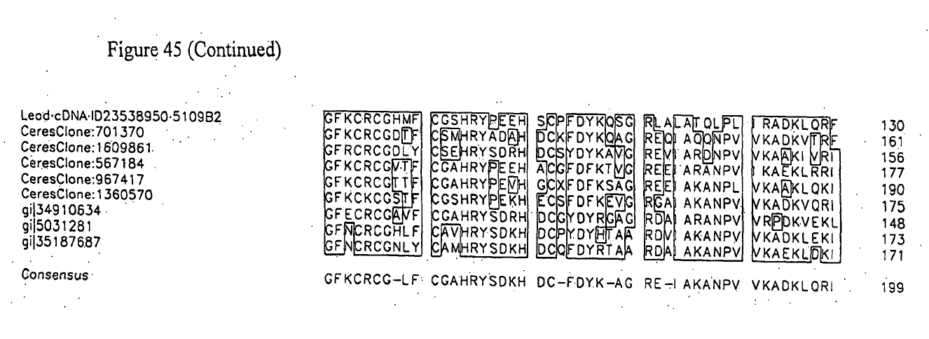

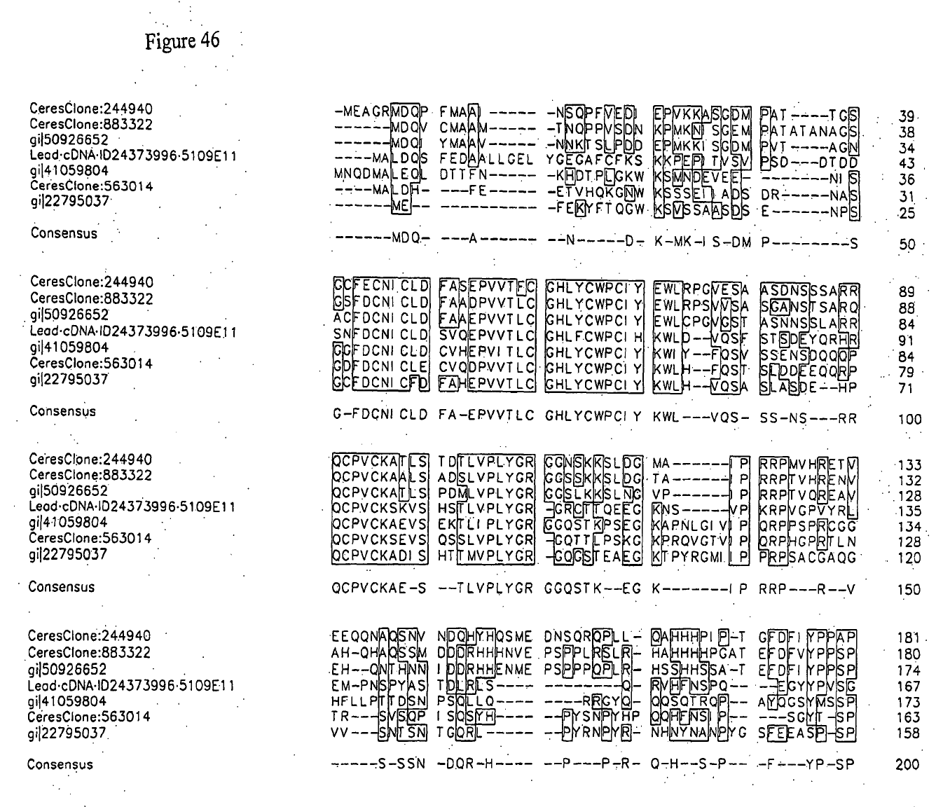

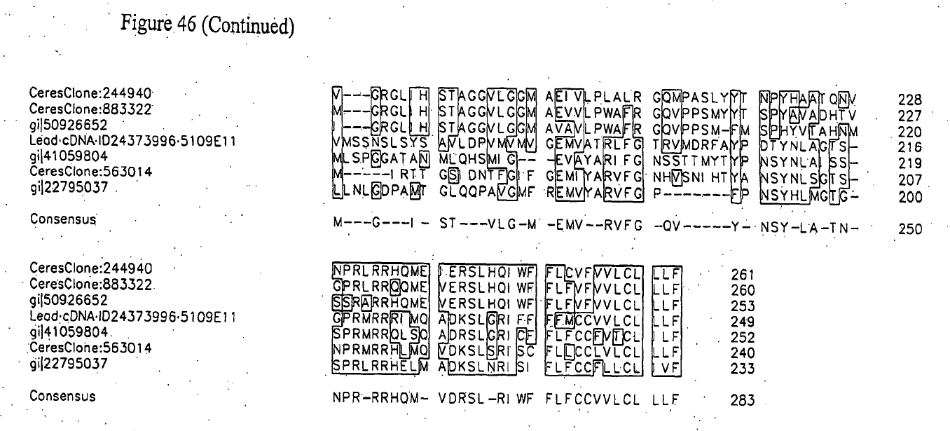

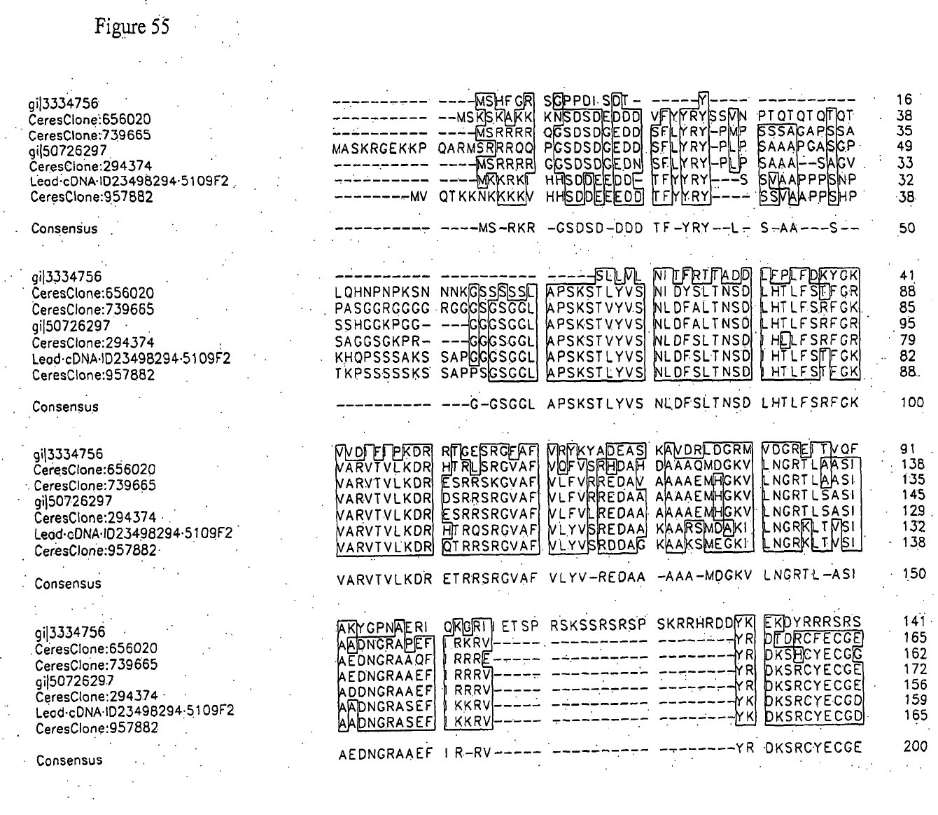

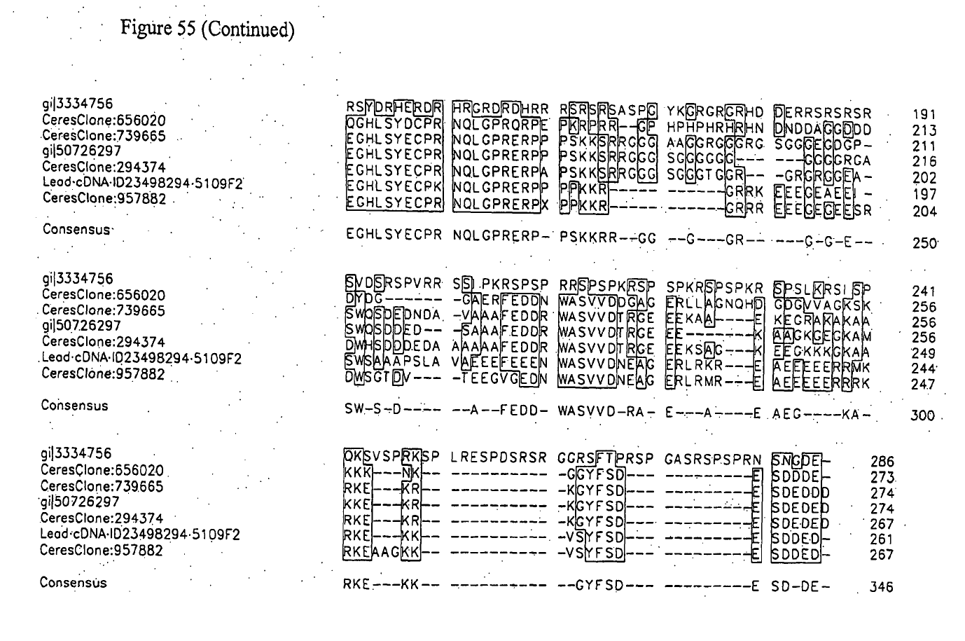

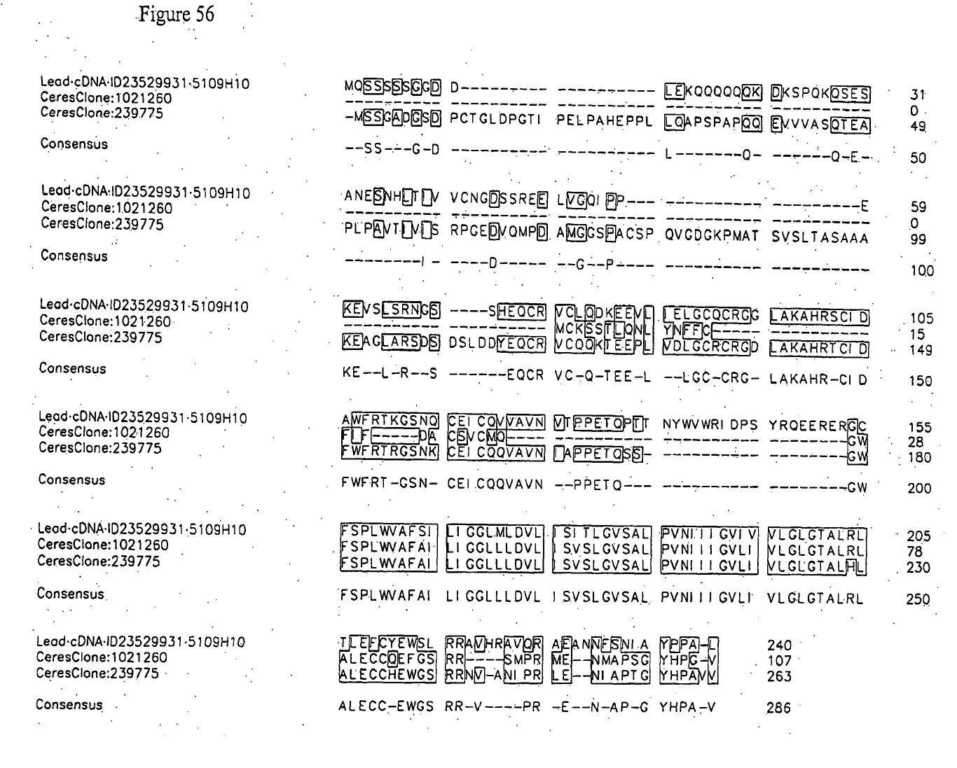

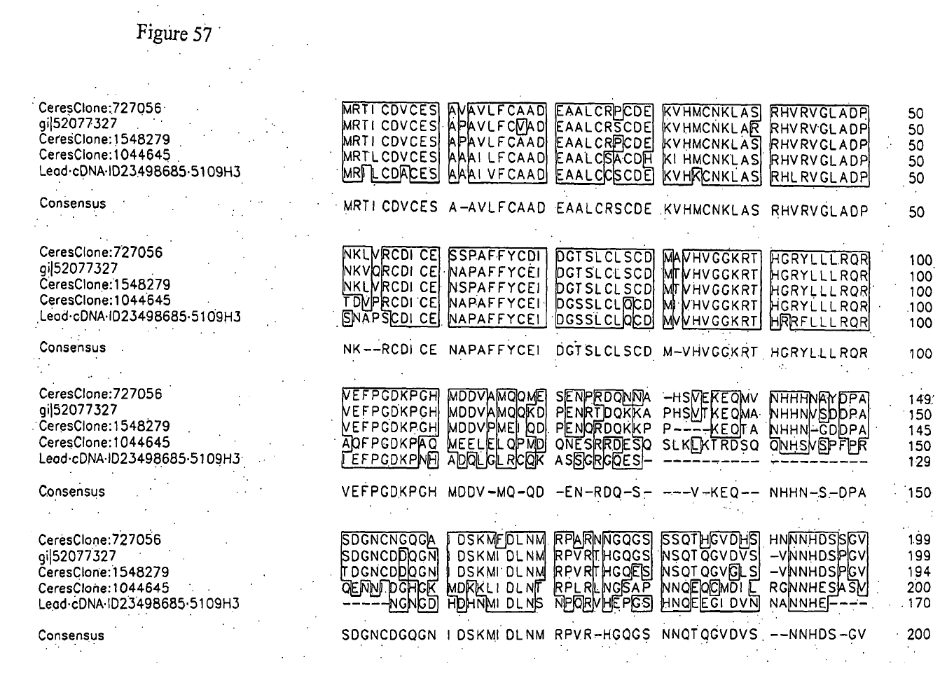

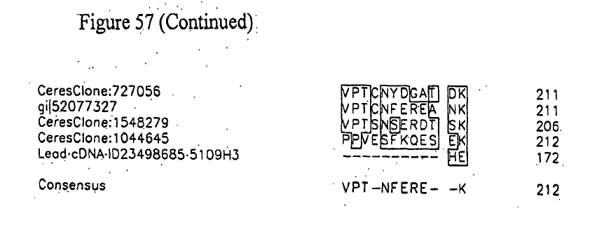

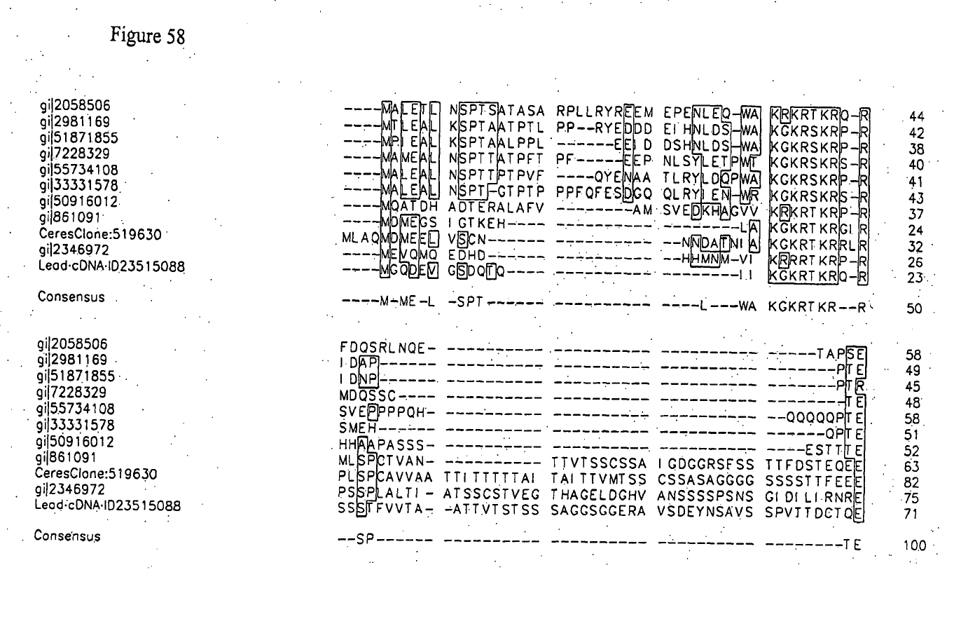

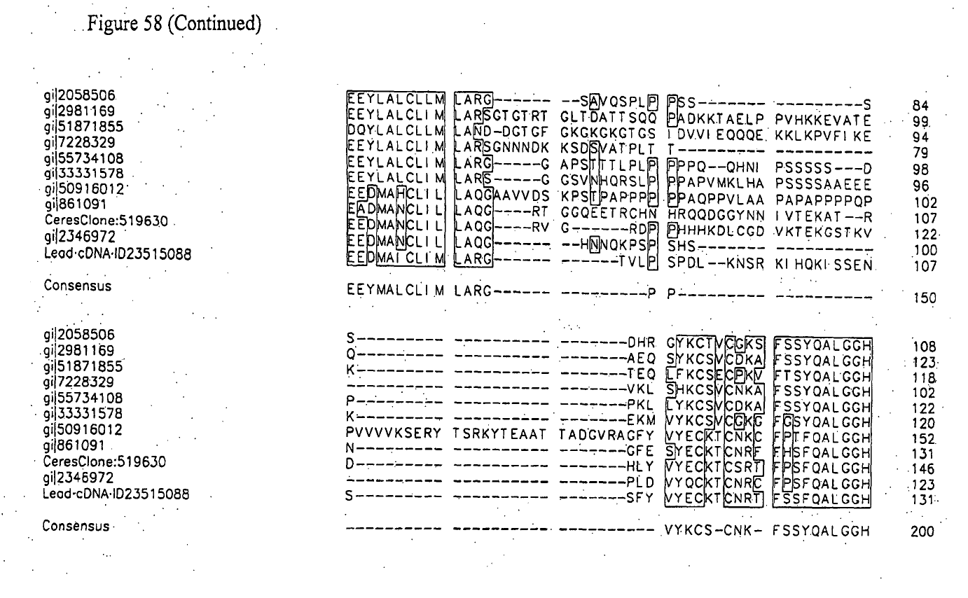

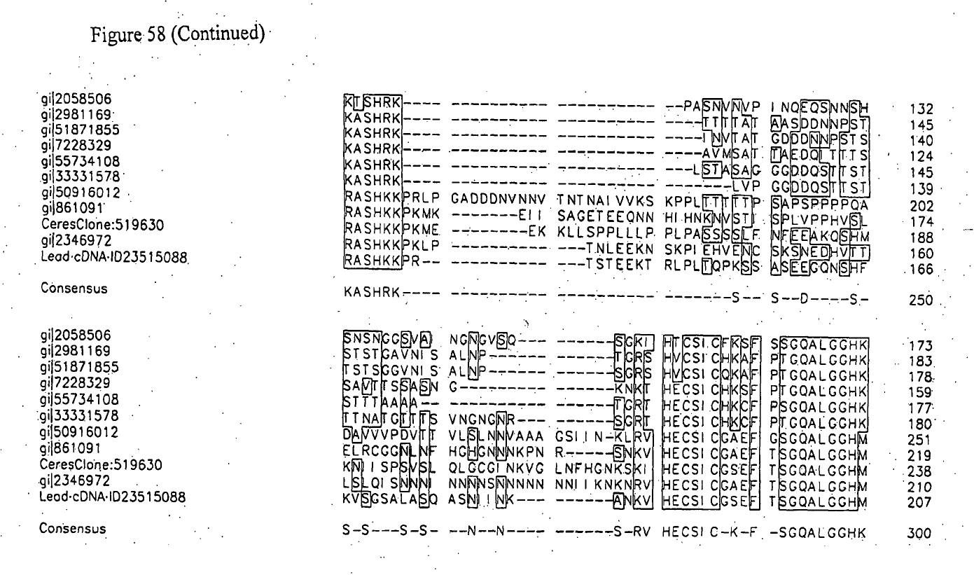

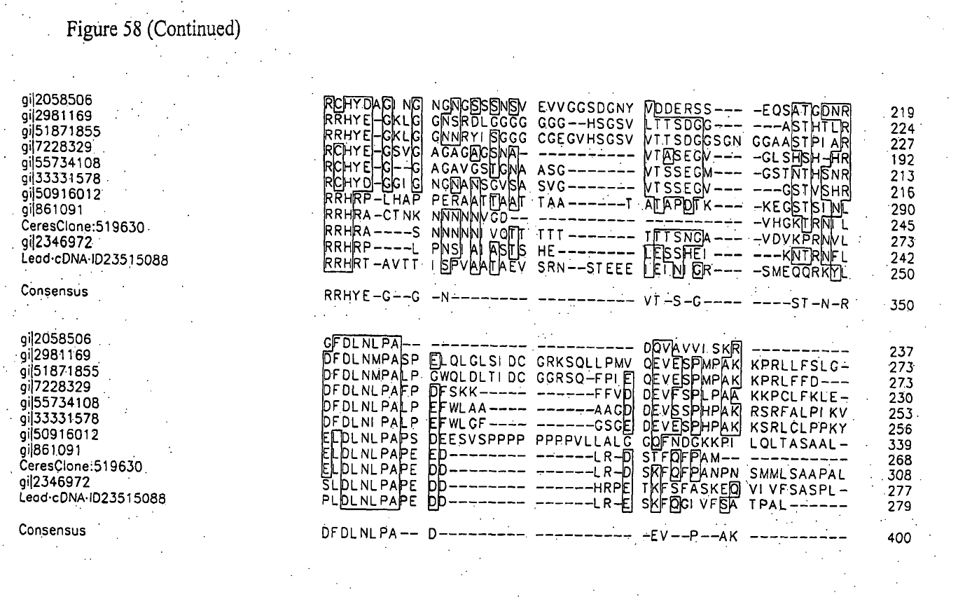

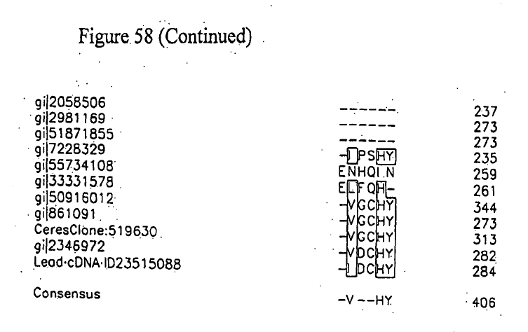

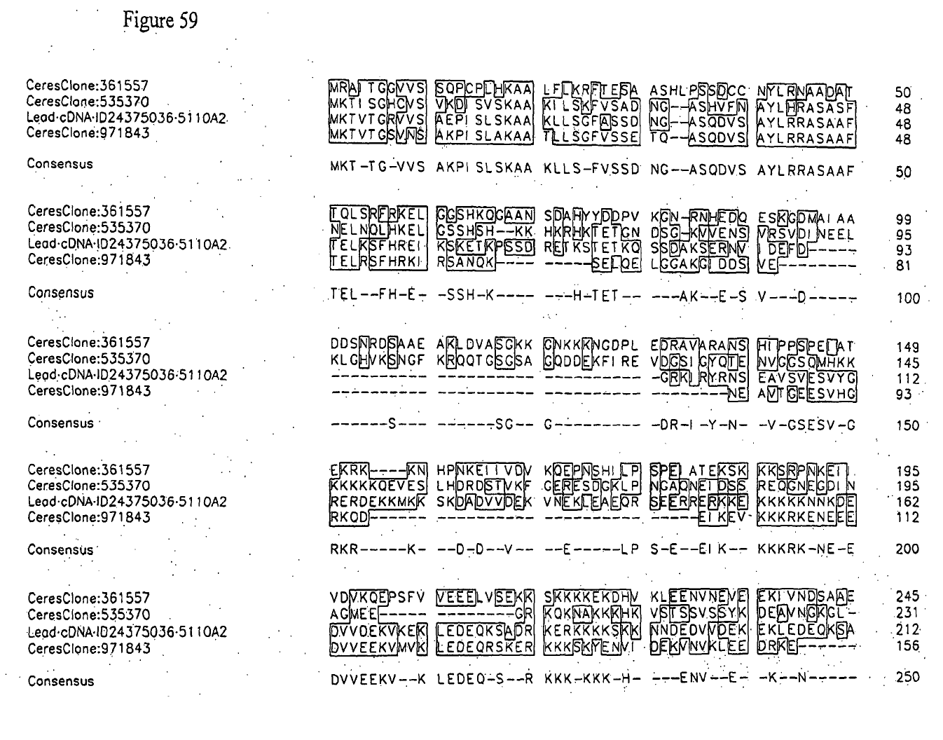

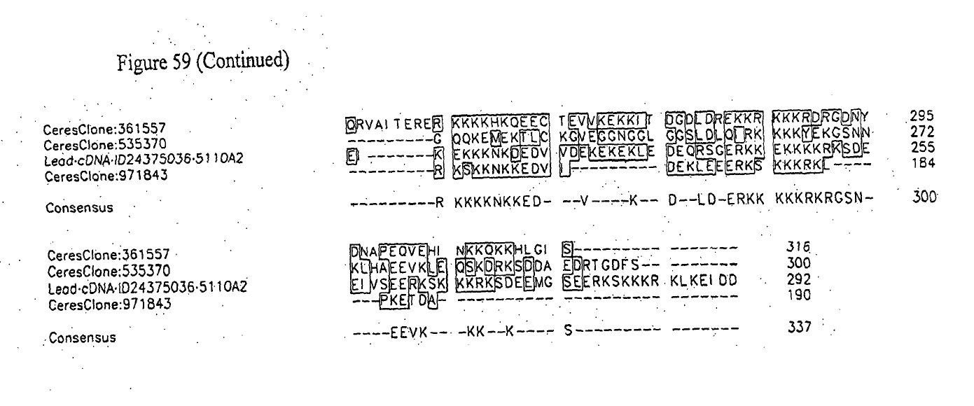

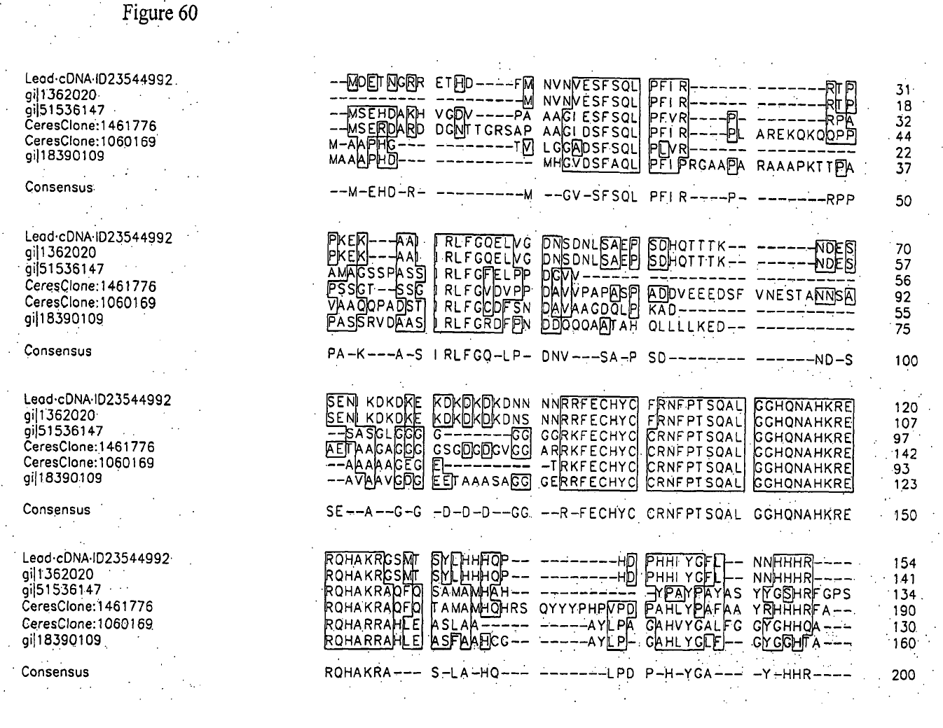

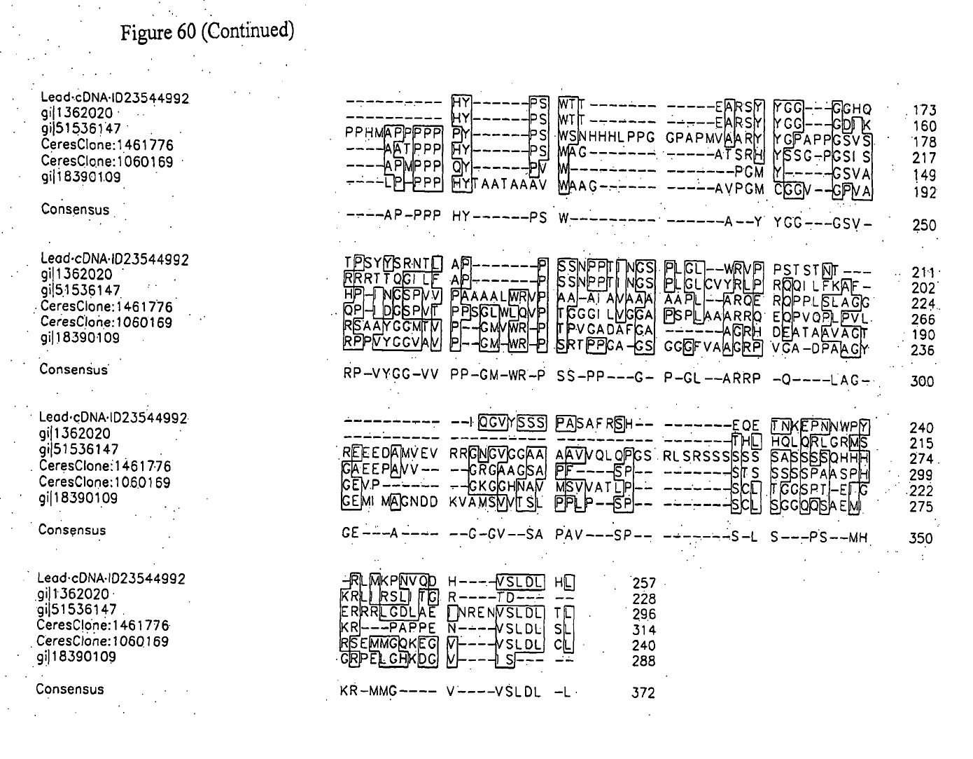

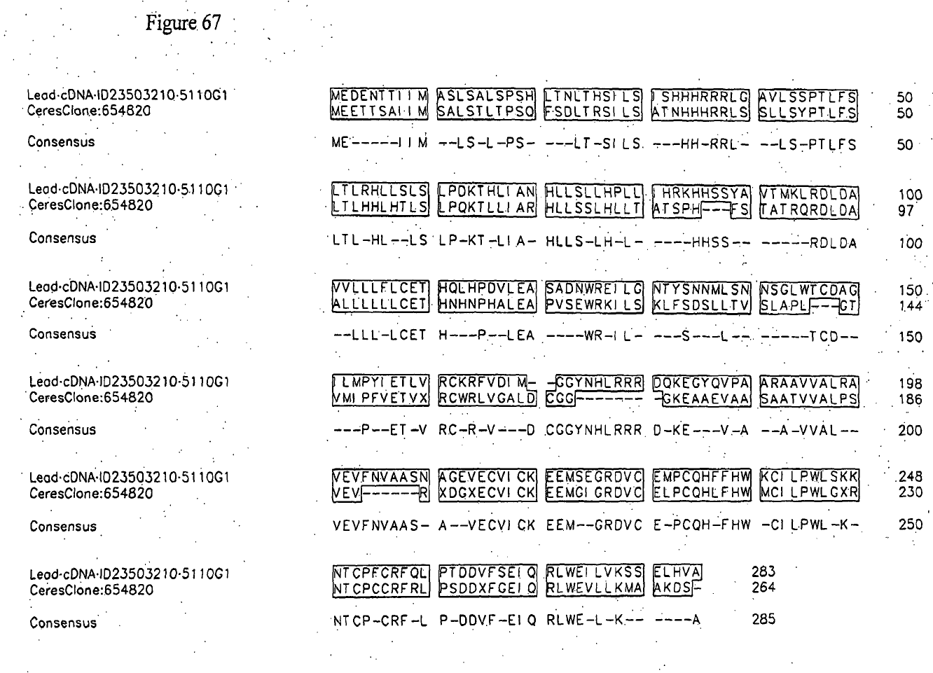

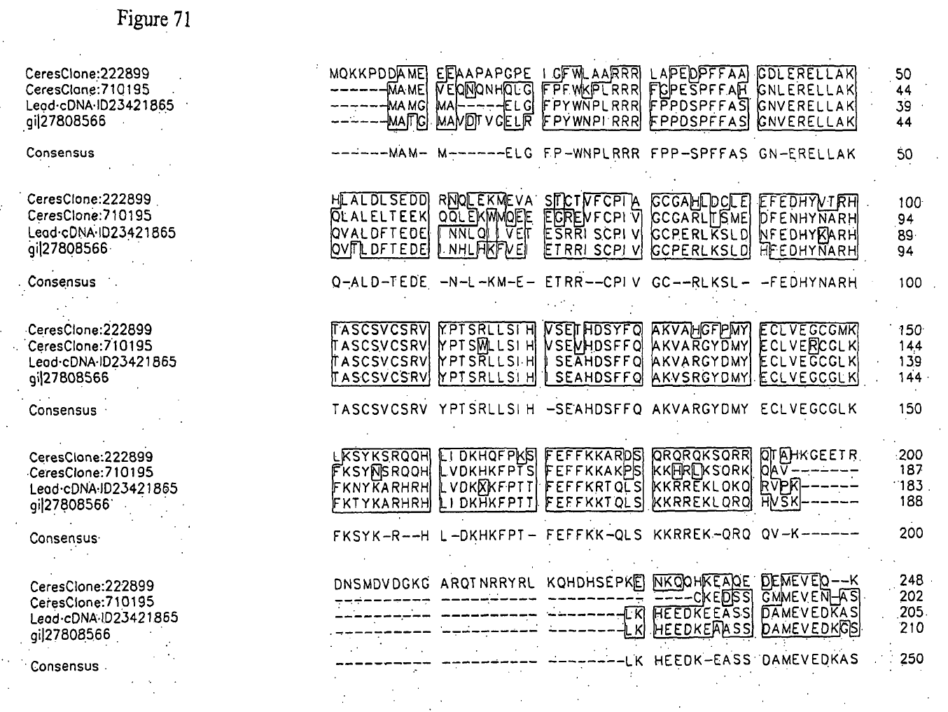

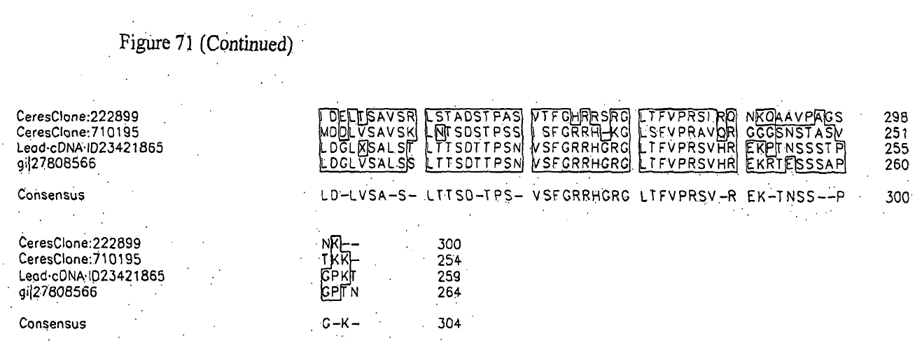

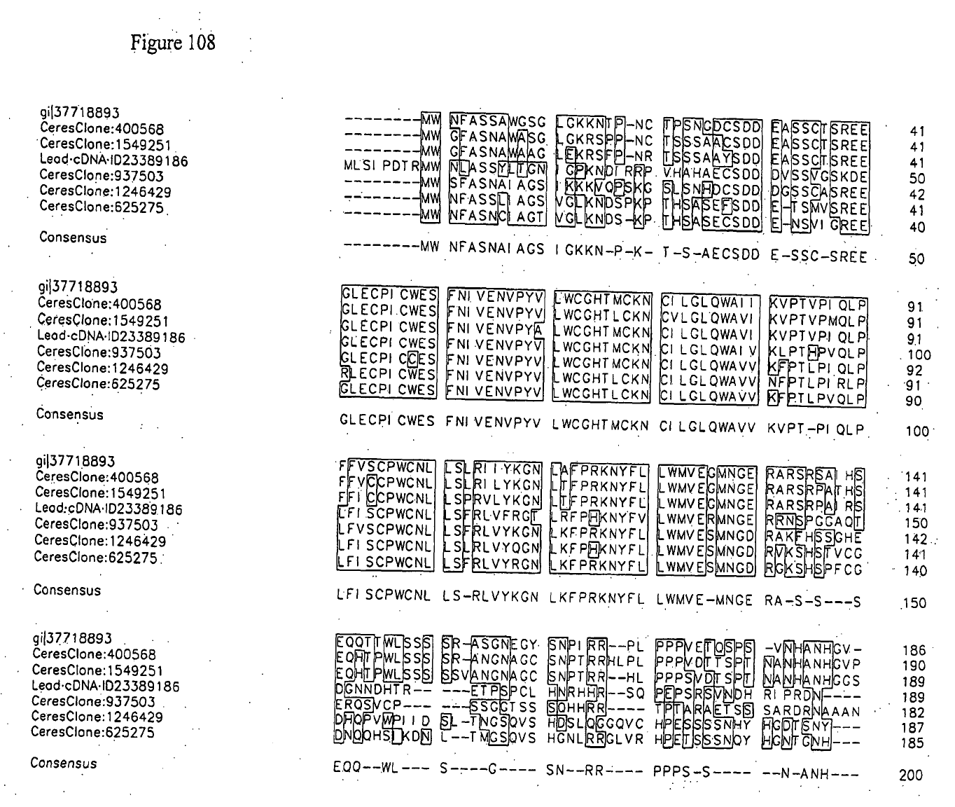

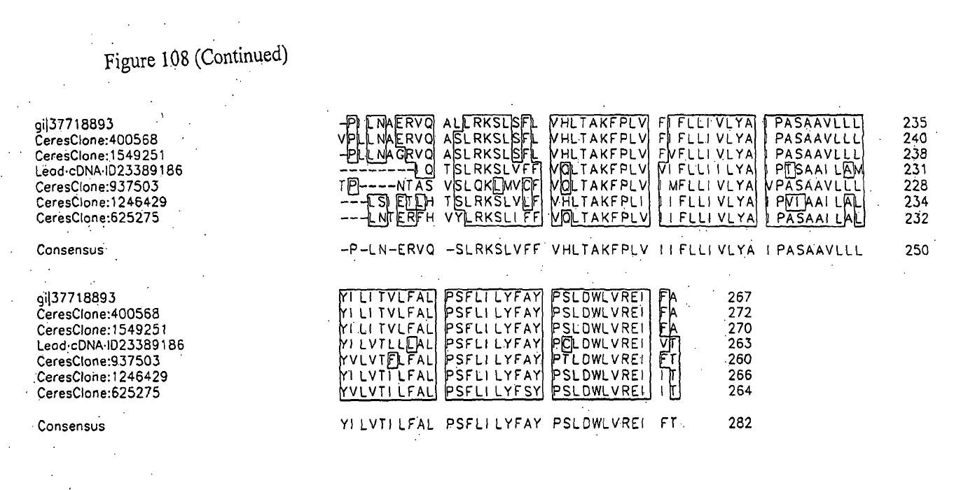

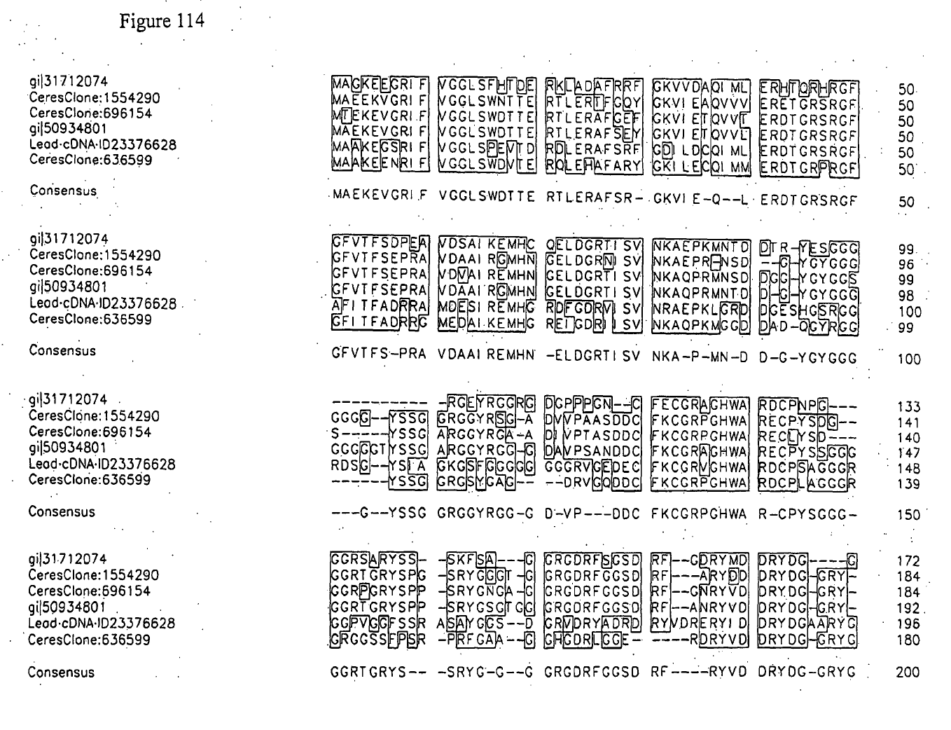

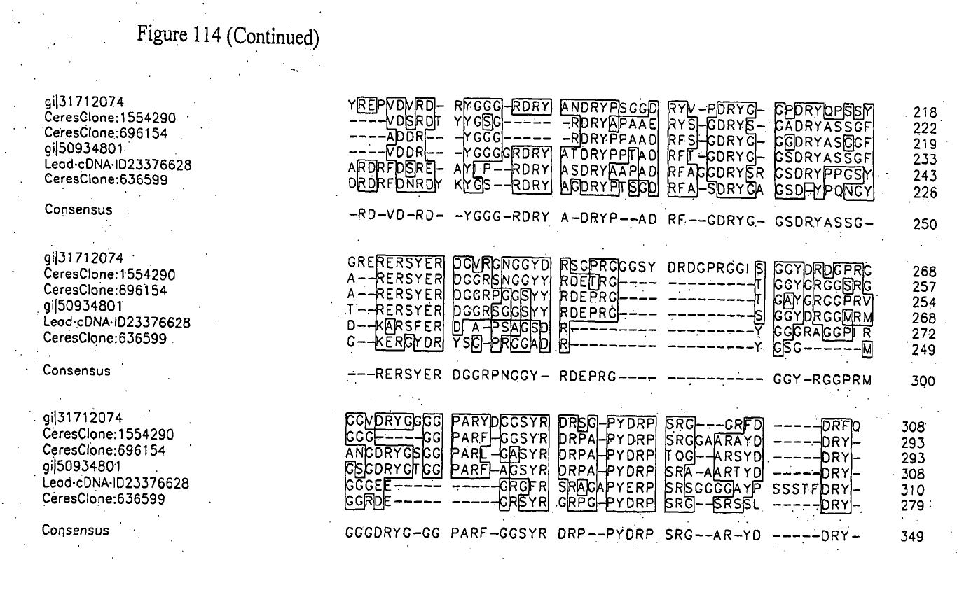

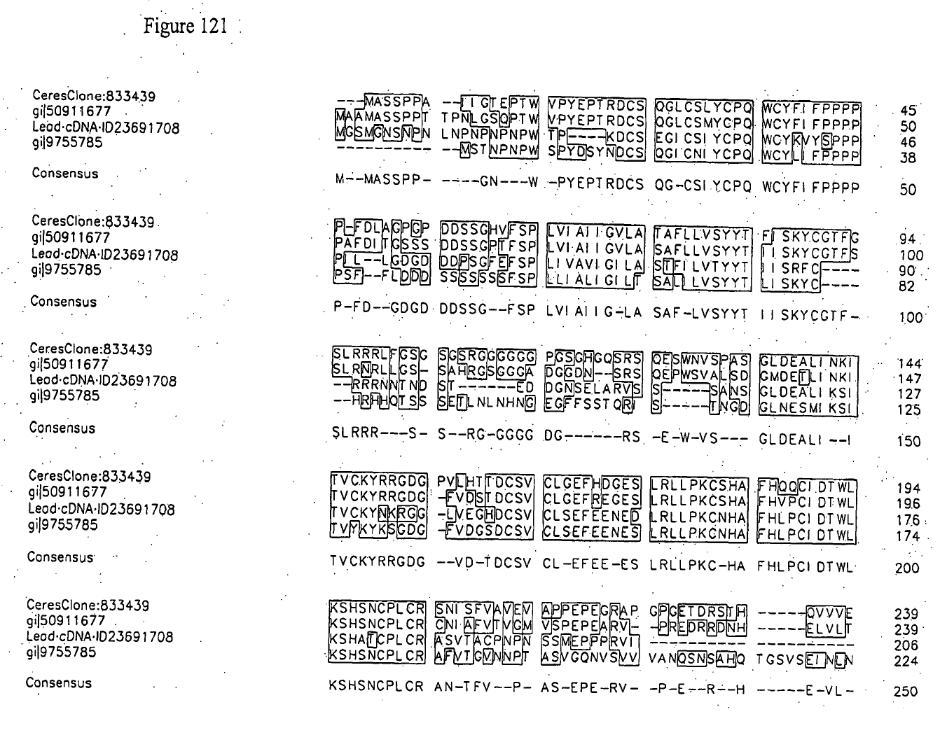

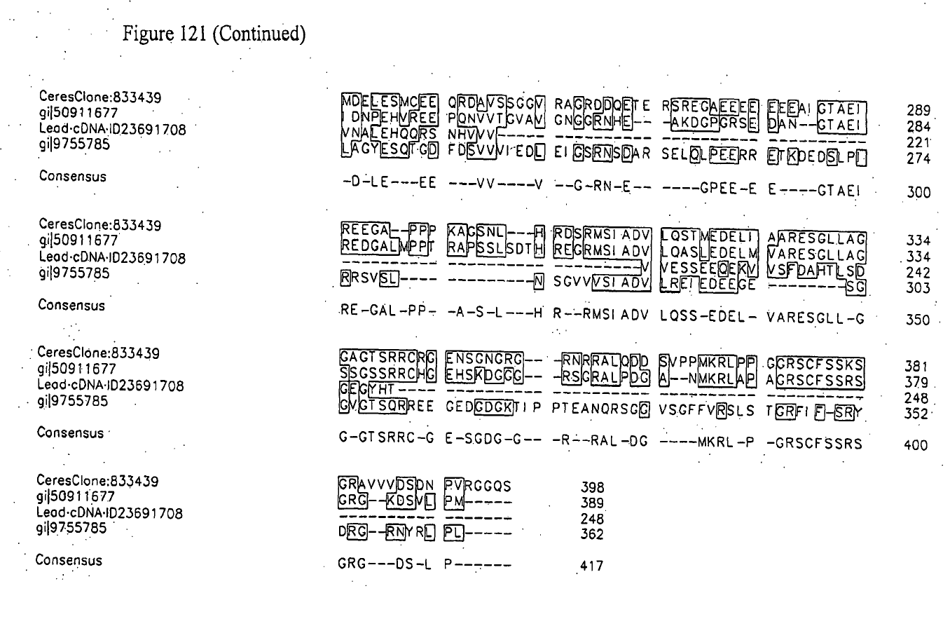

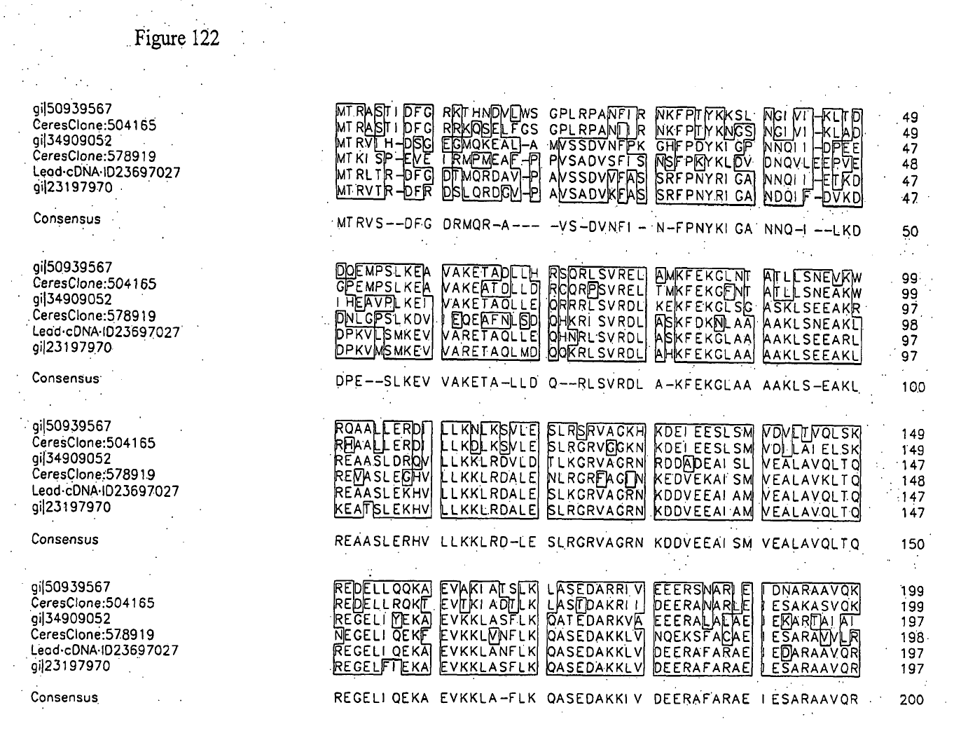

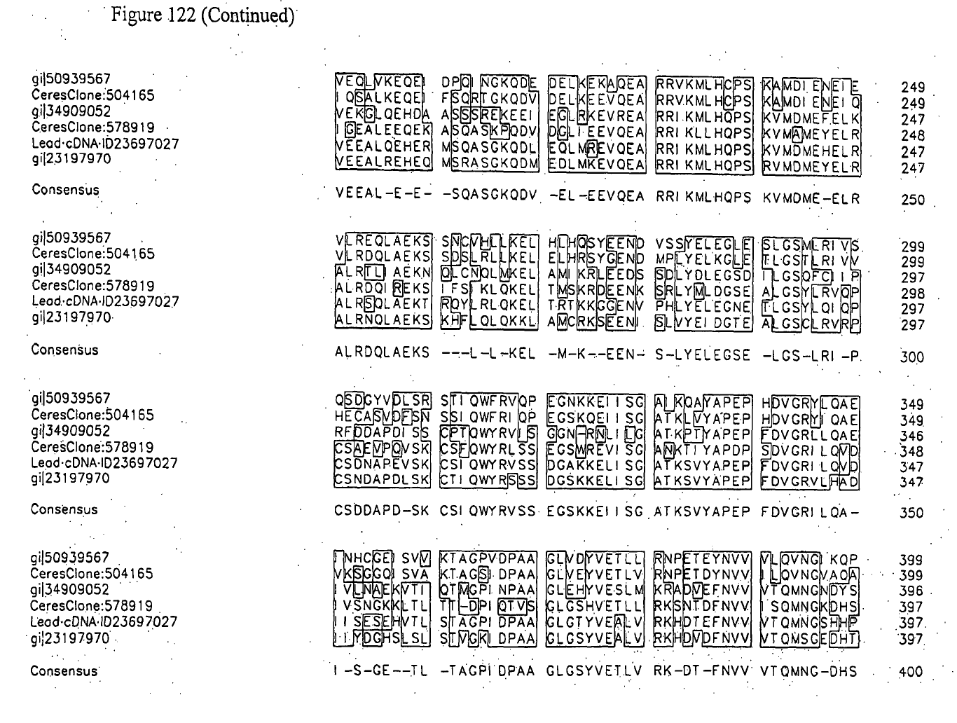

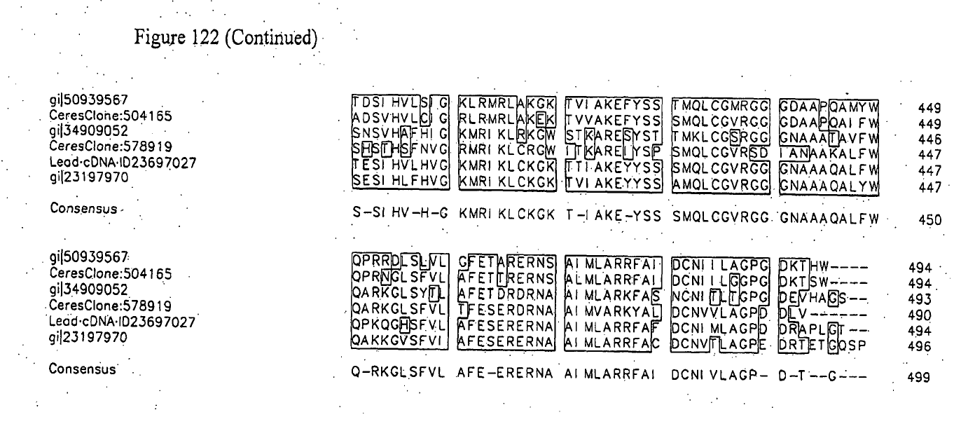

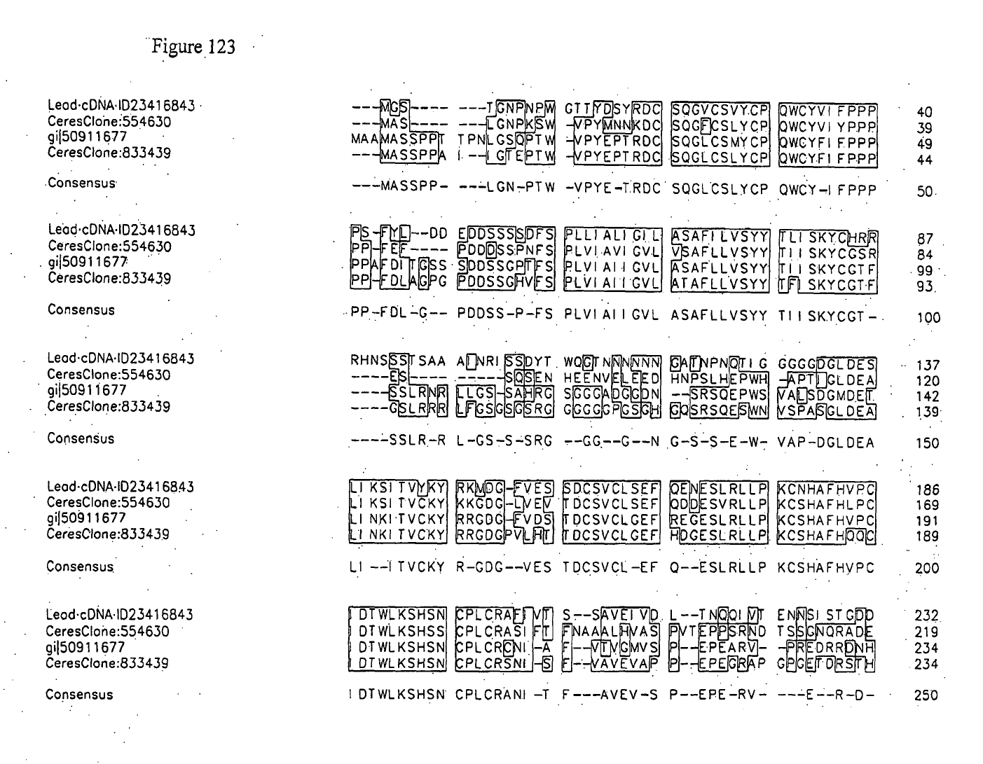

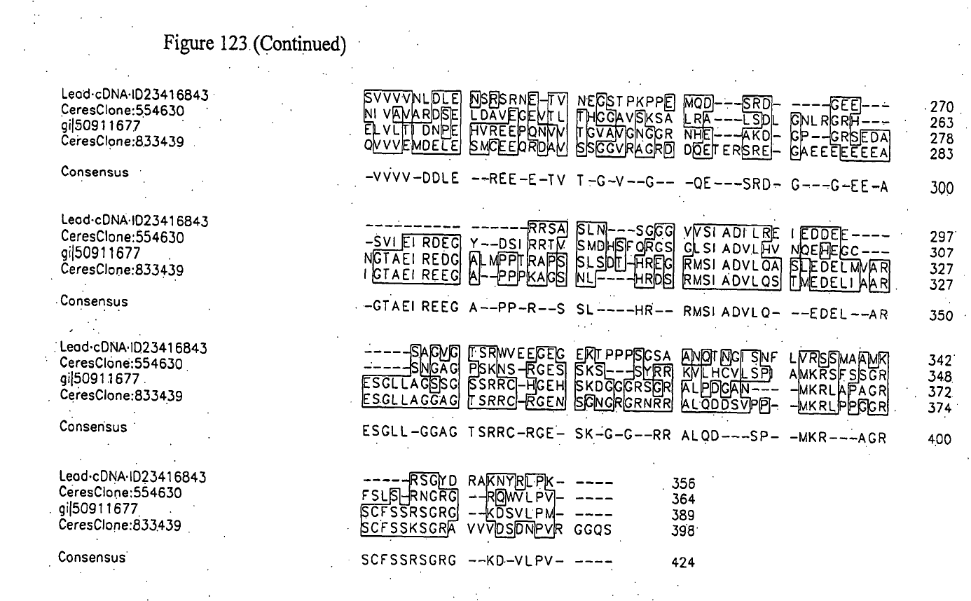

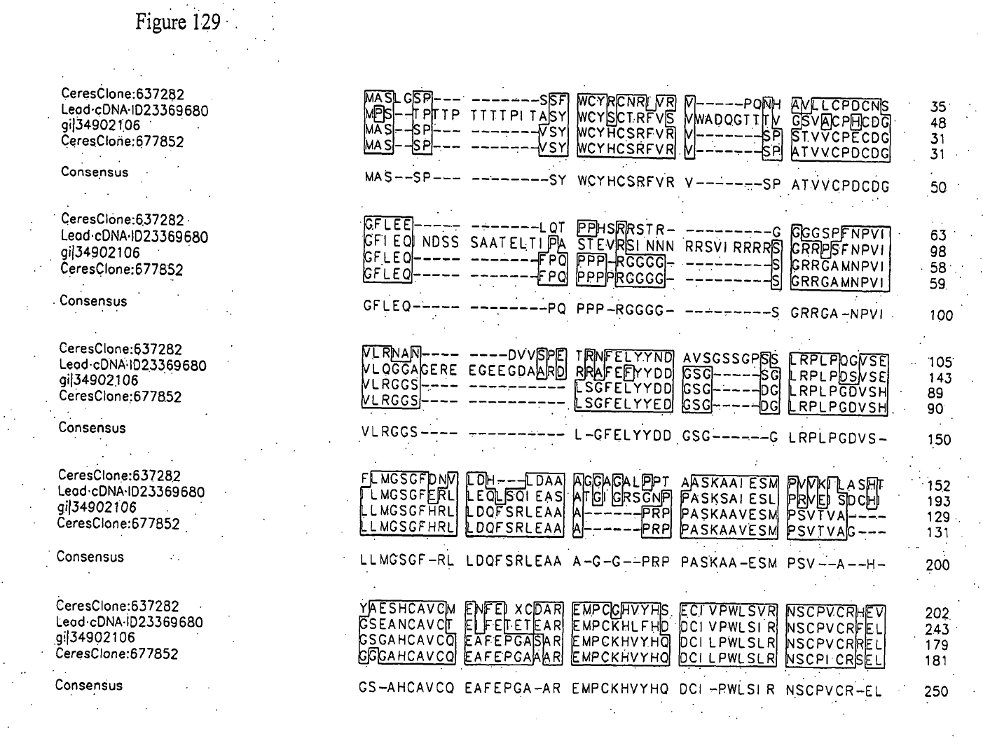

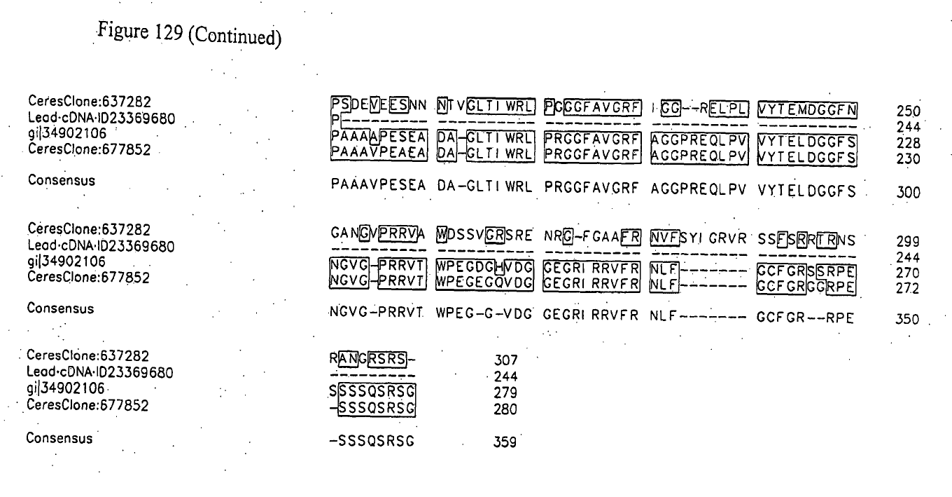

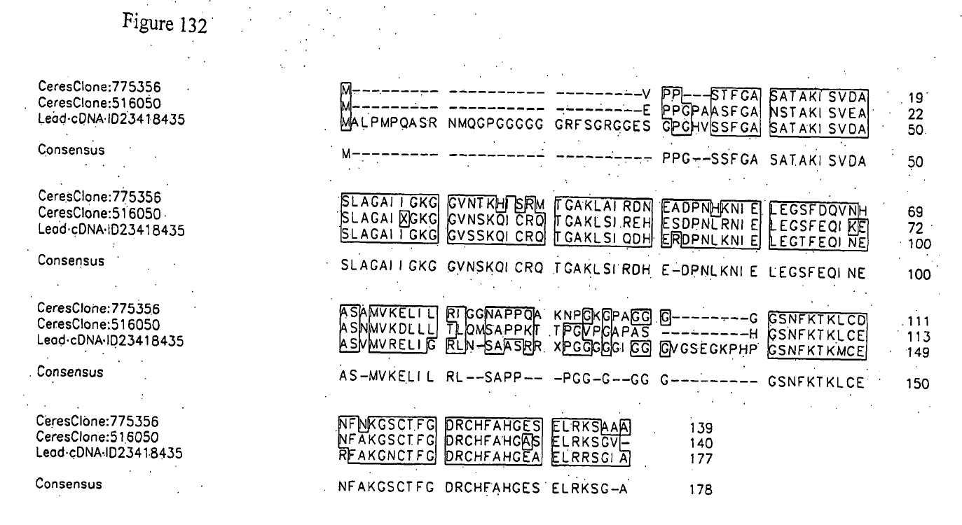

Amino acid sequences of homologs and/or orthologs of the polypeptide having the amino acid sequence set forth in SEQ ID NO:115, SEQ ID NO:168, SEQ ID NO:434, SEQ ID NO:506, SEQ ID NO:608, SEQ ID NO:695, SEQ ID NO:1119, SEQ ID NO:1243, SEQ ID NO:1255, SEQ ID NO:1335, SEQ ID NO:766, SEQ ID NO:260, SEQ ID NO:458, SEQ ID NO:141, SEQ ID NO:1369, SEQ ID NO:229, SEQ ID NO:599, SEQ ID NO:1171, SEQ ID NO:281, SEQ ID NO:494, SEQ ID NO:716, SEQ ID NO:619, SEQ ID NO:613, and SEQ ID NO:235 are provided in FIG. 4, FIG. 9, FIG. 40, FIG. 46, FIG. 56, FIG. 67, FIG. 108, FIG. 121, FIG. 123, FIG. 129, FIG. 76, FIG. 20, FIG. 42, FIG. 6, FIG. 132, FIG. 17, FIG. 55, FIG. 114, FIG. 22, FIG. 45, FIG. 71, FIG. 58, FIG. 57, and FIG. 18, respectively. Each of FIG. 4, FIG. 9, FIG. 40, FIG. 46, FIG. 56, FIG. 67, FIG. 108, FIG. 121, FIG. 123, FIG. 129, FIG. 76, FIG. 20, FIG. 42, FIG. 6, FIG. 132, FIG. 17, FIG. 55, FIG. 114, FIG. 22, FIG. 45, FIG. 71, FIG. 58, FIG. 57, and FIG. 18 also includes a consensus amino acid sequence determined by aligning homologous and/or orthologous amino acid sequences with the amino acid sequence set forth in SEQ ID NO:115, SEQ ID NO:168, SEQ ID NO:434, SEQ ID NO:506, SEQ ID NO:608, SEQ ID NO:695, SEQ ID NO:1119, SEQ ID NO:1243, SEQ ID NO:1255, SEQ ID NO:1335, SEQ ID NO:766, SEQ ID NO:260, SEQ ID NO:458, SEQ ID NO:141, SEQ ID NO:1369, SEQ ID NO:229, SEQ ID NO:599, SEQ ID NO:1171, SEQ ID NO:281, SEQ ID NO:494, SEQ ID NO:716, SEQ ID NO:619, SEQ ID NO:613, or SEQ ID NO:235, respectively.

For example, the alignment in FIG. 4 provides the amino acid sequences of cDNA ID 23663607 (SEQ ID NO:115), gi|34911396 (SEQ ID NO:116), gi|12324210 (SEQ ID NO:117), and gi|56784967 (SEQ ID NO:118). Other homologs and/or orthologs of SEQ ID NO:115 include Public GI no. 50932649 (SEQ ID NO:119).

The alignment in FIG. 9 provides the amino acid sequences of cDNA ID 23547976 (5109G9; SEQ ID NO:168), CeresClone:1358913 (SEQ ID NO:169), gi|20340241 (SEQ ID NO:170), and gi|37901055 (SEQ ID NO:171).

The alignment in FIG. 40 provides the amino acid sequences of cDNA ID 23389418 (SEQ ID NO:434), CeresClone:942980 (SEQ ID NO:435), CeresClone:1265097 (SEQ ID NO:436), CeresClone:571184 (SEQ ID NO:437), CeresClone:1052457 (SEQ ID NO:438), CeresClone:1609912 (SEQ ID NO:439), CeresClone:323551 (SEQ ID NO:440), gi|57117314 (SEQ ID NO:441), gi|50928191 (SEQ ID NO:442), gi|50253143 (SEQ ID NO:443), gi|23451086 (SEQ ID NO:444), gi|38228693 (SEQ ID NO:445), gi|37901055 (SEQ ID NO:446), gi|20340241 (SEQ ID NO:447), and gi|20152976 (SEQ ID NO:448).

The alignment in FIG. 46 provides the amino acid sequences of cDNA ID 24373996 (5109E11; SEQ ID NO:506), CeresClone:563014 (SEQ ID NO:507), gi|22795037 (SEQ ID NO:508), gi|41059804 (SEQ ID NO:509), CeresClone:883322 (SEQ ID NO:511), CeresClone:244940 (SEQ ID NO:512), and gi|50926652 (SEQ ID NO:514). Other homologs and/or orthologs of SEQ ID NO:506 include Ceres CLONE ID no. 464515 (SEQ ID NO:510) and Ceres CLONE ID no. 995691 (SEQ ID NO:513).

The alignment in FIG. 56 provides the amino acid sequence of cDNA ID 23529931 (5109H10; SEQ ID NO:608), CeresClone:1021260 (SEQ ID NO:609) and CeresClone:239775 (SEQ ID NO:610). Other homologs and/or orthologs of SEQ ID NO:608 include Ceres CLONE ID no. 316607 (SEQ ID NO:611).

The alignment in FIG. 67 provides the amino acid sequence of cDNA ID 23503210 (5110G1; SEQ ID NO:695) and CeresClone:654820 (SEQ ID NO:696).

The alignment in FIG. 108 provides the amino acid sequences of cDNA ID 23389186 (SEQ ID NO:1119), CeresClone:625275 (SEQ ID NO:1120), CeresClone:1246429 (SEQ ID NO:1121), gi|37718893 (SEQ ID NO:1122), CeresClone:937503 (SEQ ID NO:1123), CeresClone:400568 (SEQ ID NO:1124), and CeresClone:1549251 (SEQ ID NO:1125).

The alignment in FIG. 121 provides the amino acid sequences of cDNA ID 23691708 (SEQ ID NO:1243), gi|9755785 (SEQ ID NO:1244), CeresClone:833439 (SEQ ID NO:1245), and gi|50911677 (SEQ ID NO:1246).

The alignment in FIG. 123 provides the amino acid sequences of cDNA ID 23416843 (SEQ ID NO:1255), CeresClone:554630 (SEQ ID NO:1256), gi|50911677 (SEQ ID NO:1257), and CeresClone:833439 (SEQ ID NO:1259). Other homologs and/or orthologs of SEQ ID NO:1255 include Ceres CLONE ID no. 655359 (SEQ ID NO:1258).

The alignment in FIG. 129 provides the amino acid sequences of cDNA ID 23369680 (SEQ ID NO:1335), gi|34902106 (SEQ ID NO:1336), CeresClone:677852 (SEQ ID NO:1337), and CeresClone:637282 (SEQ ID NO:1338).

The alignment in FIG. 76 provides the amino acid sequences of cDNA ID 23772039 (SEQ ID NO:766) and CeresClone:864432 (SEQ ID NO:767).

The alignment in FIG. 20 provides the amino acid sequences of cDNA ID 23370190 (SEQ ID NO:260), CeresClone:287298 (SEQ ID NO:261), CeresClone:533616 (SEQ ID NO:262), gi|38196013 (SEQ ID NO:1476), gi|60460512 (SEQ ID NO:1477), gi|38260661 (SEQ ID NO:1478), CeresClone:1242254 (SEQ ID NO:1479), gi|38260624 (SEQ ID NO:1480), gi|34906436 (SEQ ID NO:1481), gi|56605376 (SEQ ID NO:1482), CeresClone:673872 (SEQ ID NO:1483), and CeresClone:997341 (SEQ ID NO:1484).

The alignment in FIG. 42 provides the amino acid sequences of cDNA ID 23365920 (SEQ ID NO:458), gi|5616313 (SEQ ID NO:459), CeresClone:751992 (SEQ ID NO:460), CeresClone:833872 (SEQ ID NO:461), gi|62901482 (SEQ ID NO:462), gi|34906988 (SEQ ID NO:463), and CeresClone:1579587 (SEQ ID NO:464).

The alignment in FIG. 6 provides the amino acid sequences of cDNA ID 23447462 (5109E7; SEQ ID NO:141) and gi|50923905 (SEQ ID NO:142).

The alignment in FIG. 132 provides the amino acid sequences of cDNA ID 23418435 (SEQ ID NO:1369), CeresClone:516050 (SEQ ID NO:1370) and CeresClone:775356 (SEQ ID NO:1371). Other homologs and/or orthologs of SEQ ID NO:1369 include Ceres CLONE ID no. 472196 (SEQ ID NO:1372).

The alignment in FIG. 17 provides the amino acid sequences of cDNA ID 13579142 (5111E1; SEQ ID NO:229), CeresClone:463860 (SEQ ID NO:230), gi|50927857 (SEQ ID NO:231), CeresClone:296774 (SEQ ID NO:232), and CeresClone:843076 (SEQ ID NO:233).

The alignment in FIG. 55 provides the amino acid sequences of cDNA ID 23498294 (5109F2; SEQ ID NO:599), CeresClone:957882 (SEQ ID NO:600), gi|50726297 (SEQ ID NO:601), CeresClone:739665 (SEQ ID NO:602), CeresClone:294374 (SEQ ID NO:603), CeresClone:656020 (SEQ ID NO:605), and gi|3334756 (SEQ ID NO:606). Other homologs and/or orthologs include Ceres CLONE ID no. 372141 (SEQ ID NO:604).

The alignment in FIG. 114 provides the amino acid sequences of cDNA ID 23376628 (SEQ ID NO:1171), CeresClone:636599 (SEQ ID NO:1172), gi|50934801 (SEQ ID NO:1173), gi|31712074 (SEQ ID NO:1174), CeresClone:696154 (SEQ ID NO:1175), and CeresClone:1554290 (SEQ ID NO:1176).

The alignment in FIG. 22 provides the amino acid sequences of cDNA ID 23364997 (SEQ ID NO:281), gi|11994583 (SEQ ID NO:282), CeresClone:1021269 (SEQ ID NO:283), CeresClone:592400 (SEQ ID NO:284), CeresClone:302213 (SEQ ID NO:285), and gi|50900102 (SEQ ID NO:286).

The alignment in FIG. 45 provides the amino acid sequences of cDNA ID 23538950 (5109B2; SEQ ID NO:494), CeresClone:567184 (SEQ ID NO:496), CeresClone:967417 (SEQ ID NO:497), CeresClone:1360570 (SEQ ID NO:498), CeresClone:701370 (SEQ ID NO:499), gi|5031281 (SEQ ID NO:500), gi|35187687 (SEQ ID NO:501), gi|34910634 (SEQ ID NO:503), and CeresClone:1609861 (SEQ ID NO:504). Other homologs and/or orthologs of SEQ ID NO:494 include Ceres CLONE ID no. 111288 (SEQ ID NO:495) and Ceres CLONE ID no. 849111 (SEQ ID NO:502).

The alignment in FIG. 71 provides the amino acid sequences of cDNA ID 23421865 (SEQ ID NO:716), gi|27808566 (SEQ ID NO:717), CeresClone:710195 (SEQ ID NO:718), and CeresClone:222899 (SEQ ID NO:719).

The alignment in FIG. 58 provides the amino acid sequences of cDNA ID 23515088 (SEQ ID NO:619), gi|50916012 (SEQ ID NO:620), gi|861091 (SEQ ID NO:621), gi|2346972 (SEQ ID NO:622), CeresClone:519630 (SEQ ID NO:623), gi|7228329 (SEQ ID NO:624), gi|2981169 (SEQ ID NO:625), gi|55734108 (SEQ ID NO:626), gi|33331578 (SEQ ID NO:627), gi|51871855 (SEQ ID NO:628), and gi|2058506 (SEQ ID NO:629). Other homologs and/or orthologs of SEQ ID NO:619 include Public GI no. 2058504 (SEQ ID NO:630).

The alignment in FIG. 57 provides the amino acid sequences of cDNA ID 23498685 (5109H3; SEQ ID NO:613), gi|52077327 (SEQ ID NO:614), CeresClone:1044645 (SEQ ID NO:615), CeresClone:1548279 (SEQ ID NO:616), and CeresClone:727056 (SEQ ID NO:617).

The alignment in FIG. 18 provides the amino acid sequences of cDNA ID 23365150 (SEQ ID NO:235), gi|4996642 (SEQ ID NO:236), gi|50253202 (SEQ ID NO:237), gi|47900733 (SEQ ID NO:238), gi|7489820 (SEQ ID NO:239), gi|4996644 (SEQ ID NO:240), gi|37051125 (SEQ ID NO:241), CeresClone:543840 (SEQ ID NO:242), gi|33332411 (SEQ ID NO:243), and gi|42556524 (SEQ ID NO:244).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:116-119, SEQ ID NOs:169-171, SEQ ID NOs:435-448, SEQ ID NOs:507-514, SEQ ID NOs:609-611, SEQ ID NO:696, SEQ ID NOs:1120-1125, SEQ ID NOs:1244-1246, SEQ ID NOs:1256-1259, SEQ ID NOs:1336-1338, SEQ ID NO:767, SEQ ID NOs:261-262, SEQ ID NOs:1476-1484, SEQ ID NOs:459-464, SEQ ID NO:142, SEQ ID NO:1370-1372, SEQ ID NOs:230-233, SEQ ID NOs:600-606, SEQ ID NOs:1172-1176, SEQ ID NOs:282-286, SEQ ID NOs:495-504, SEQ ID NOs:717-719, SEQ ID NOs:620-630, SEQ ID NOs:614-617, SEQ ID NOs:236-244, or the consensus sequence set forth in FIG. 4, FIG. 9, FIG. 40, FIG. 46, FIG. 56, FIG. 67, FIG. 108, FIG. 121, FIG. 123, FIG. 129, FIG. 76, FIG. 20, FIG. 42, FIG. 6, FIG. 132, FIG. 17, FIG. 55, FIG. 114, FIG. 22, FIG. 45, FIG. 71, FIG. 58, FIG. 57, or FIG. 18.

A regulatory protein can contain an SRF-TF domain characteristic of an SRF-type transcription factor (DNA binding and dimerization domain) polypeptide. Human serum response factor (SRF) is a ubiquitous nuclear protein important for cell proliferation and differentiation. SRF function is essential for transcriptional regulation of numerous growth-factor-inducible genes, such as the c-fos oncogene and muscle-specific actin genes. A core domain of about 90 amino acids is sufficient for the activities of DNA binding, dimerization, and interaction with accessory factors. Within the core is a DNA binding region, designated the MADS box that is highly similar to many eukaryotic regulatory proteins, including the Agamous and Deficiens families of plant homeotic proteins. SEQ ID NO:123, SEQ ID NO:563, SEQ ID NO:590, SEQ ID NO:679, SEQ ID NO:698, and SEQ ID NO:822 set forth the amino acid sequences of DNA clones, identified herein as cDNA ID 23522096 (SEQ ID NO:122), cDNA ID 23502516 (SEQ ID NO:562), cDNA ID 23519948 (SEQ ID NO:589), cDNA ID 23554709 (SEQ ID NO:678), cDNA ID 23494809 (SEQ ID NO:697), and cDNA ID 23495742 (SEQ ID NO:821), respectively, that are predicted to encode SRF-type transcription factor (DNA binding and dimerization domain) polypeptides.

In some cases, a regulatory protein can contain an SRF-TF domain and a K-box region. Moreover, a K-box region is commonly found associated with SRF-type transcription factors. The K-box is predicted to have a coiled-coil structure and a role in multimer formation. SEQ ID NO:216, SEQ ID NO:472, SEQ ID NO:532, SEQ ID NO:748, SEQ ID NO:889, SEQ ID NO:946, SEQ ID NO:964, SEQ ID NO:1102, and SEQ ID NO:1226 set forth the amino acid sequences of DNA clones, identified herein as cDNA ID 4984839 (SEQ ID NO:215), cDNA ID 23783423 (SEQ ID NO:471), cDNA ID 12680548 (SEQ ID NO:531), cDNA ID 23773450 (SEQ ID NO:747), cDNA ID 23556617 (SEQ ID NO:888), cDNA ID 23766279 (SEQ ID NO:945), cDNA ID 23746932 (SEQ ID NO:963), cDNA ID 23448883 (SEQ ID NO:1101), and cDNA ID 23747378 (SEQ ID NO:1225), respectively, that are predicted to encode SRF-type transcription factor polypeptides having a K-box region.

A regulatory protein can comprise the amino acid sequence set forth in SEQ ID NO:123, SEQ ID NO:563, SEQ ID NO:590, SEQ ID NO:679, SEQ ID NO:698, SEQ ID NO:822, SEQ ID NO:216, SEQ ID NO:472, SEQ ID NO:532, SEQ ID NO:748, SEQ ID NO:889, SEQ ID NO:946, SEQ ID NO:964, SEQ ID NO:1102, or SEQ ID NO:1226. Alternatively, a regulatory protein can be a homolog, ortholog, or variant of the polypeptide having the amino acid sequence set forth in SEQ ID NO:123, SEQ ID NO:563, SEQ ID NO:590, SEQ ID NO:679, SEQ ID NO:698, SEQ ID NO:822, SEQ ID NO:216, SEQ ID NO:472, SEQ ID NO:532, SEQ ID NO:748, SEQ ID NO:889, SEQ ID NO:946, SEQ ID NO:964, SEQ ID NO:1102, or SEQ ID NO:1226. For example, a regulatory protein can have an amino acid sequence with at least 30% sequence identity, e.g., 31%, 35%, 40%, 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:123, SEQ ID NO:563, SEQ ID NO:590, SEQ ID NO:679, SEQ ID NO:698, SEQ ID NO:822, SEQ ID NO:216, SEQ ID NO:472, SEQ ID NO:532, SEQ ID NO:748, SEQ ID NO:889, SEQ ID NO:946, SEQ ID NO:964, SEQ ID NO:1102, or SEQ ID NO:1226.

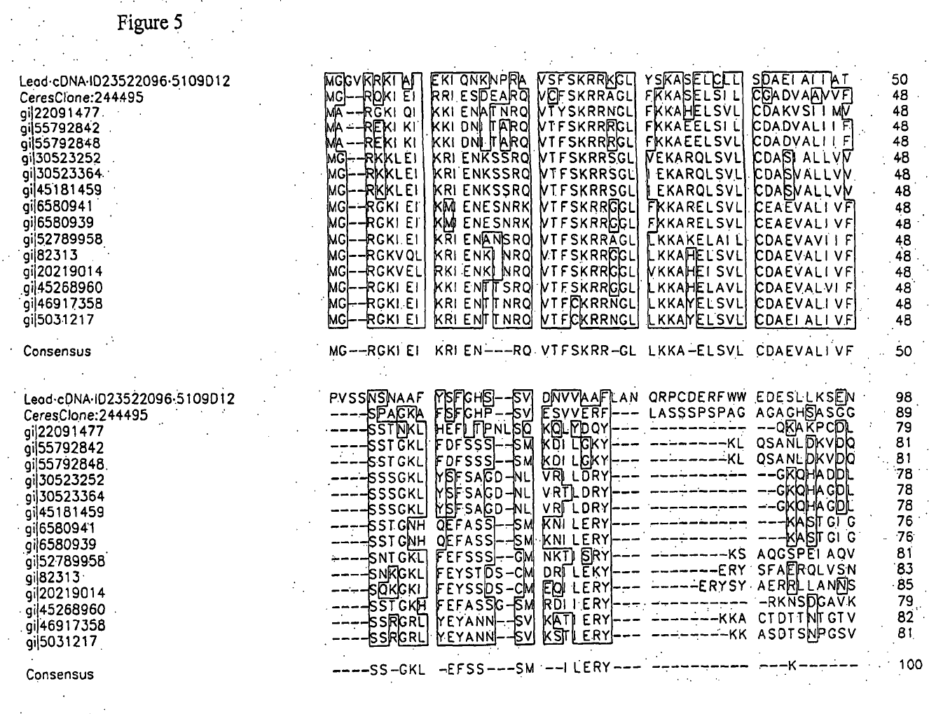

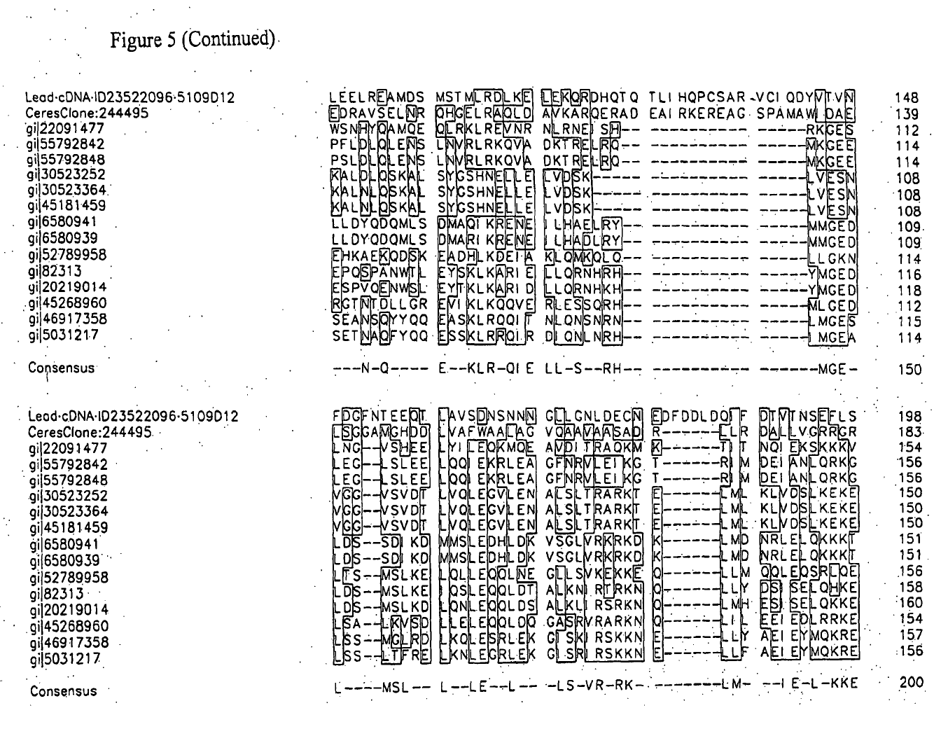

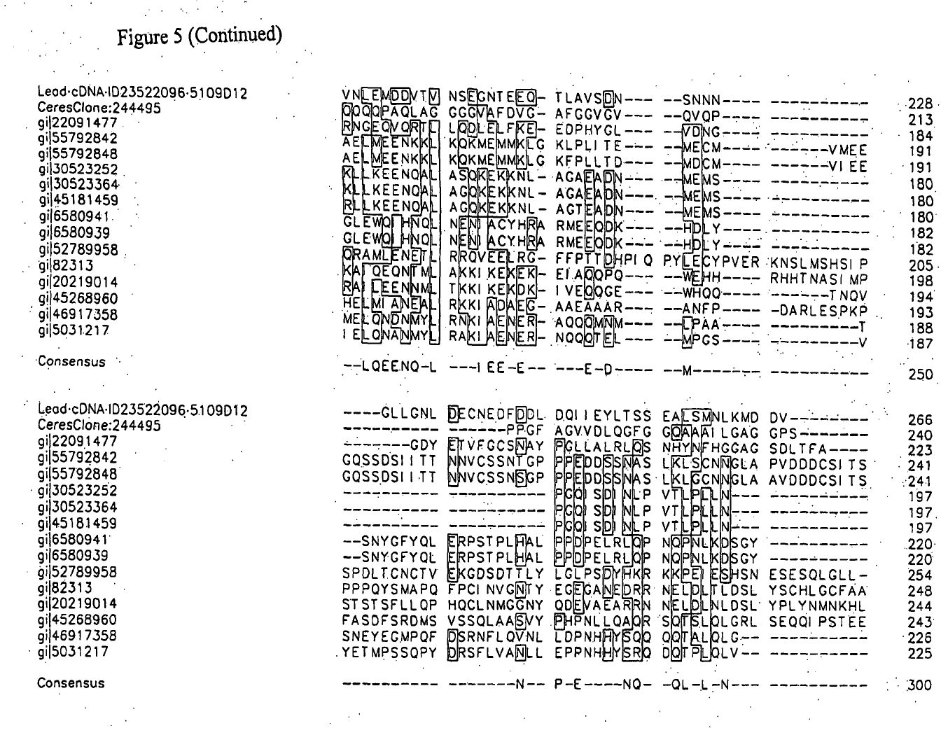

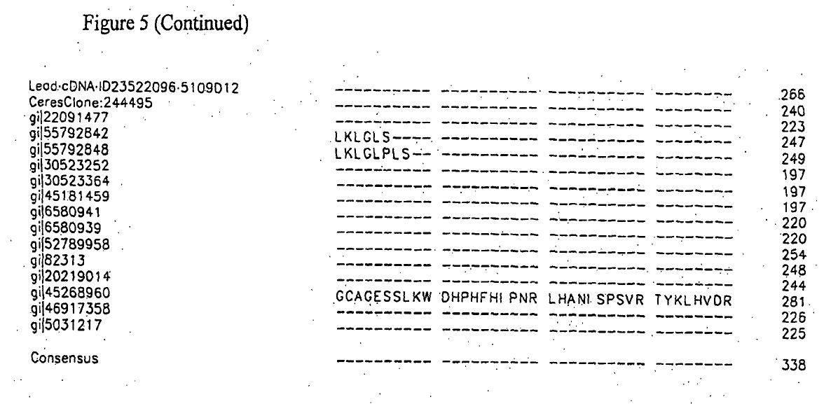

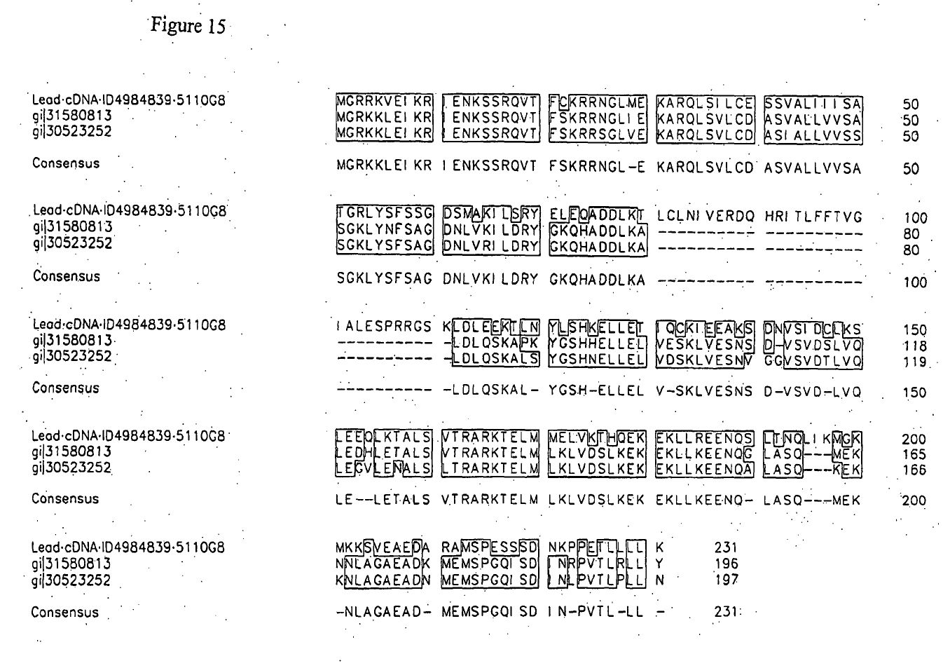

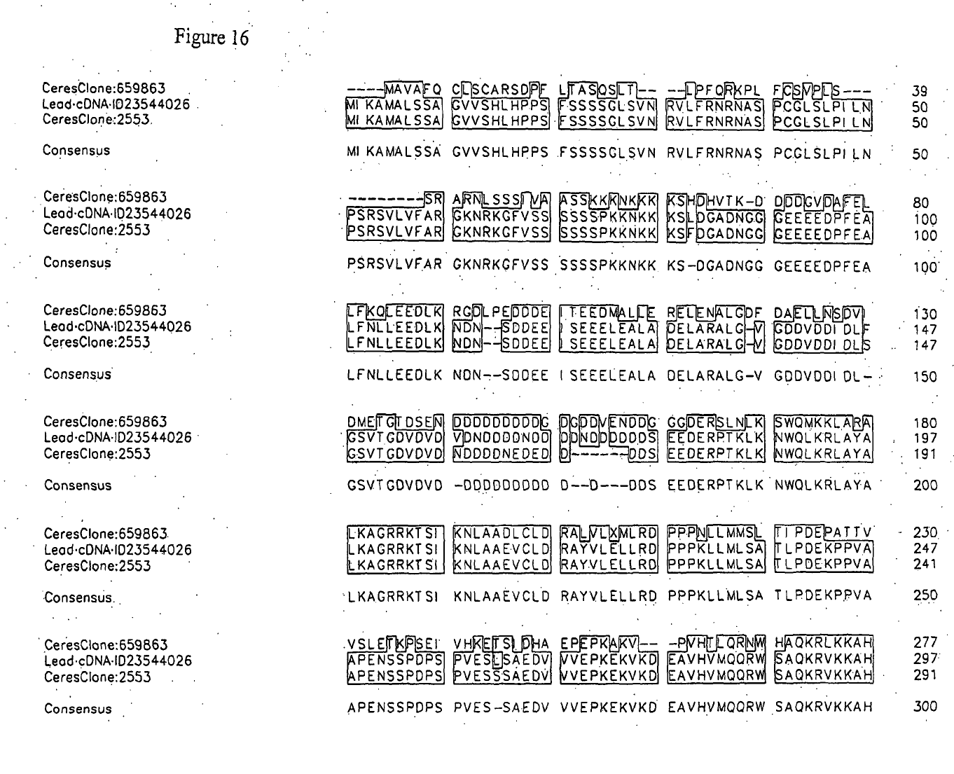

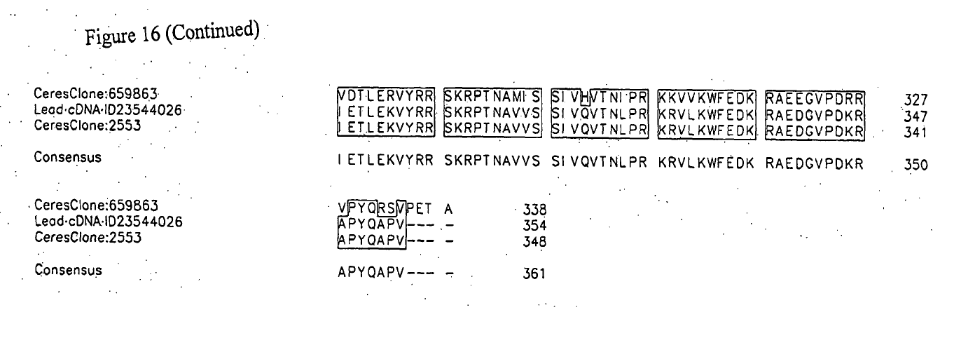

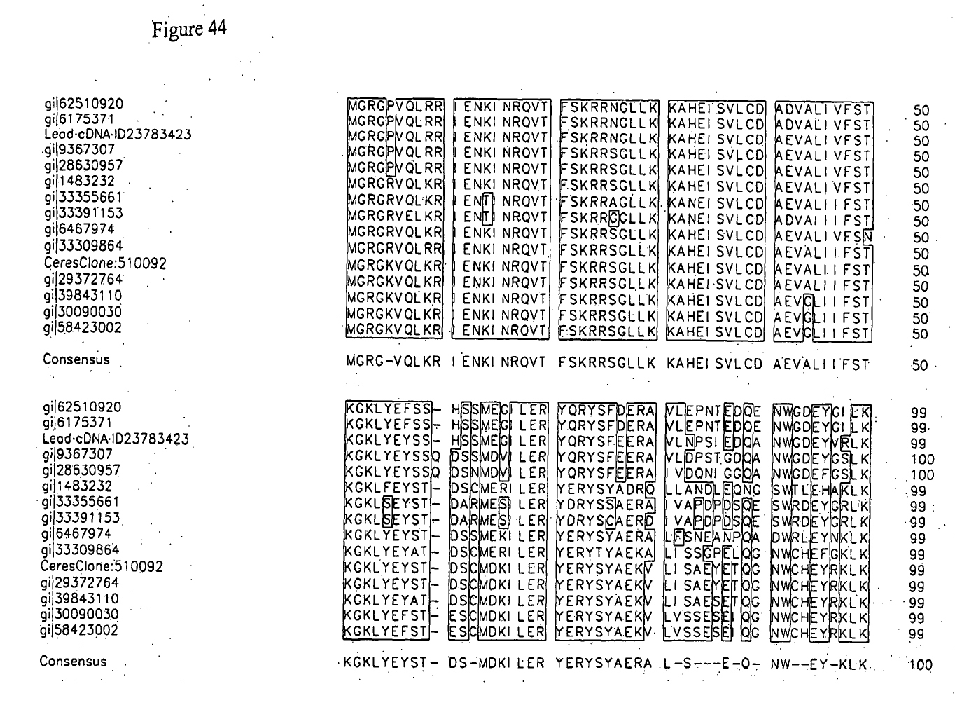

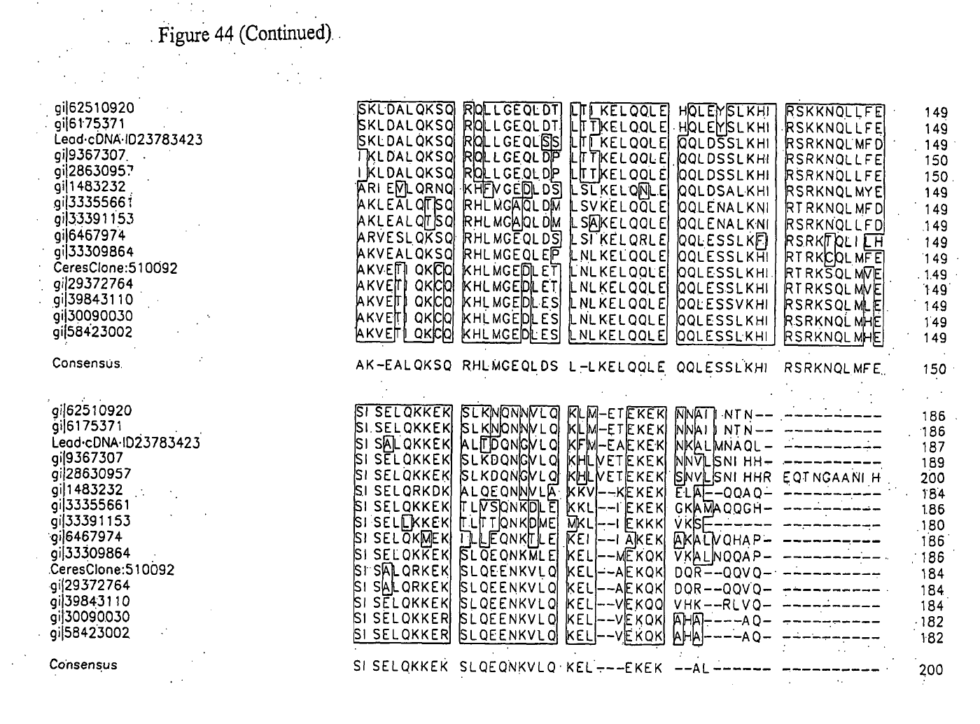

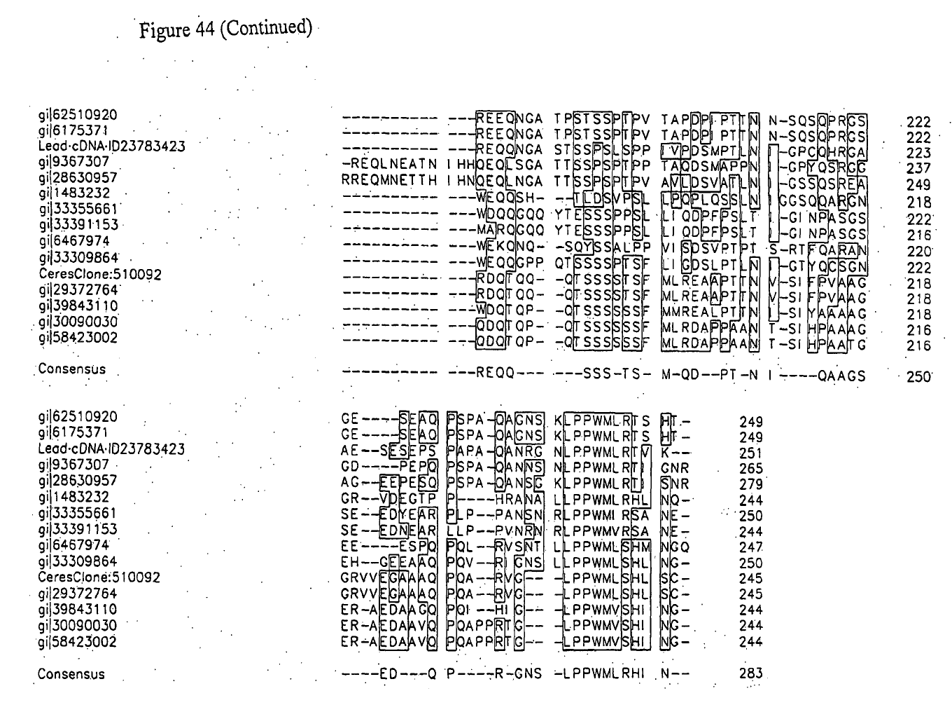

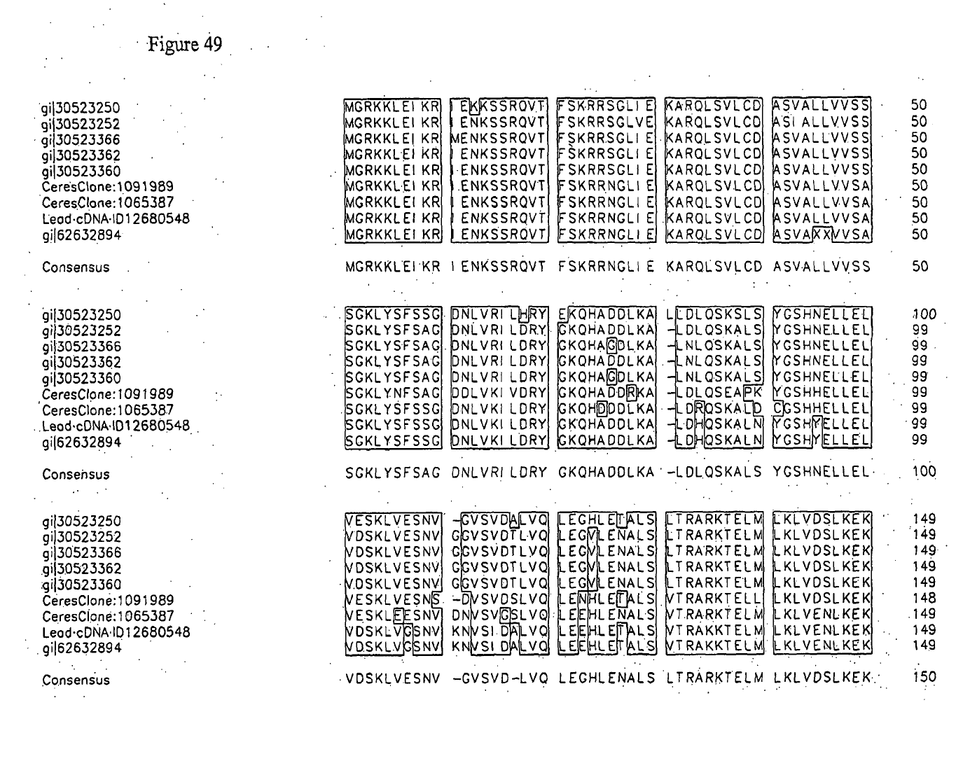

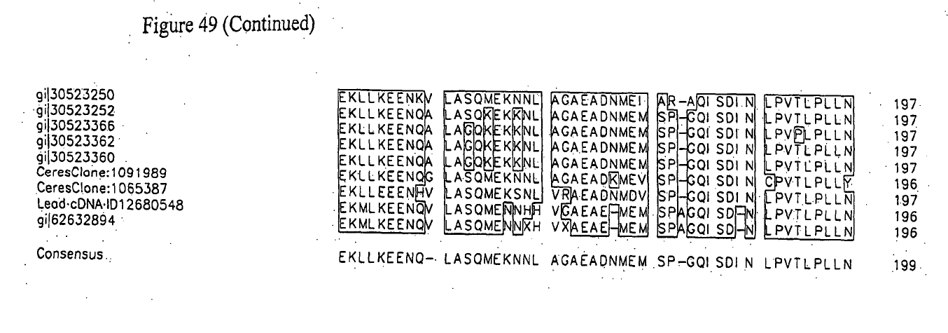

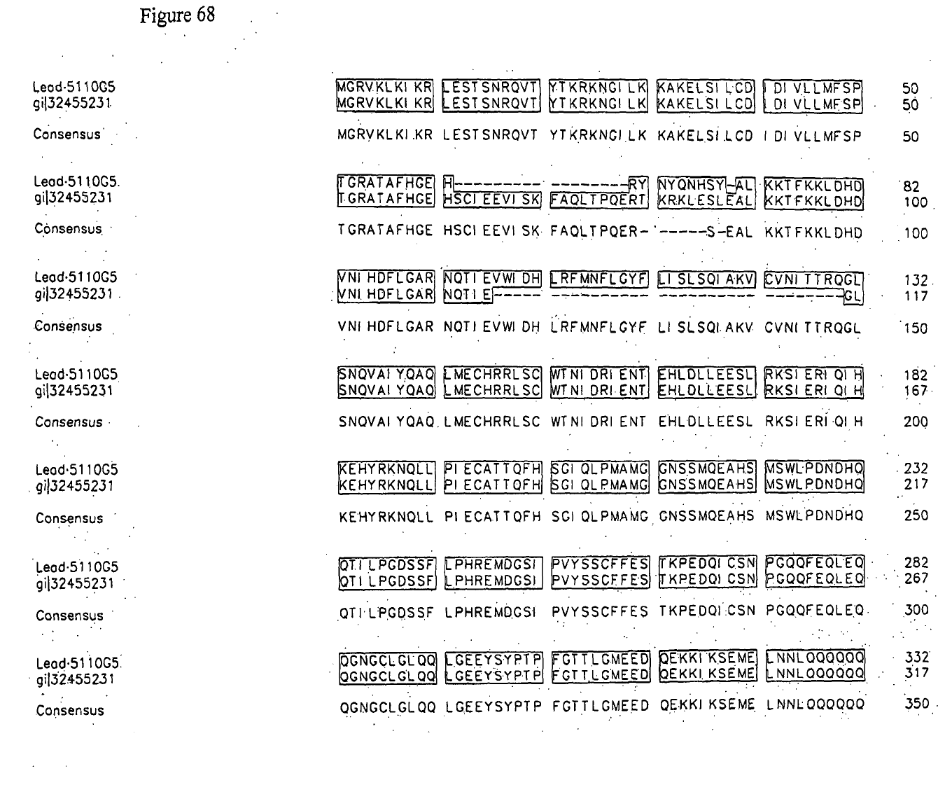

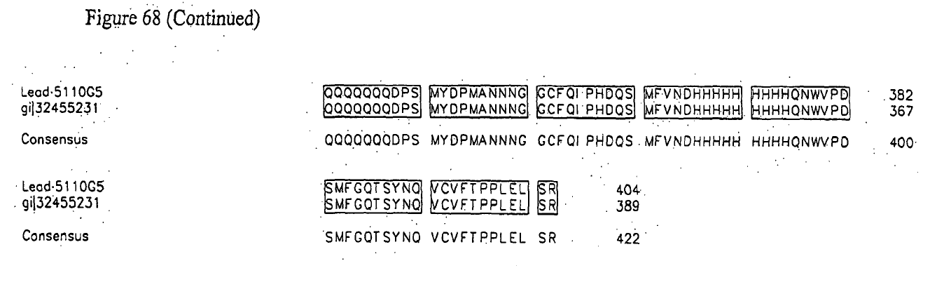

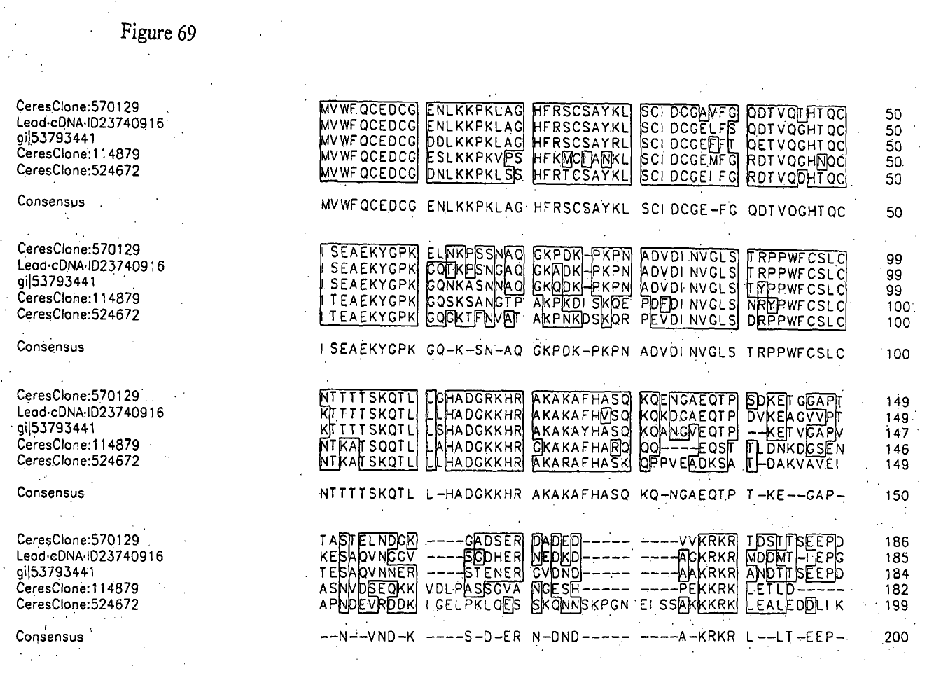

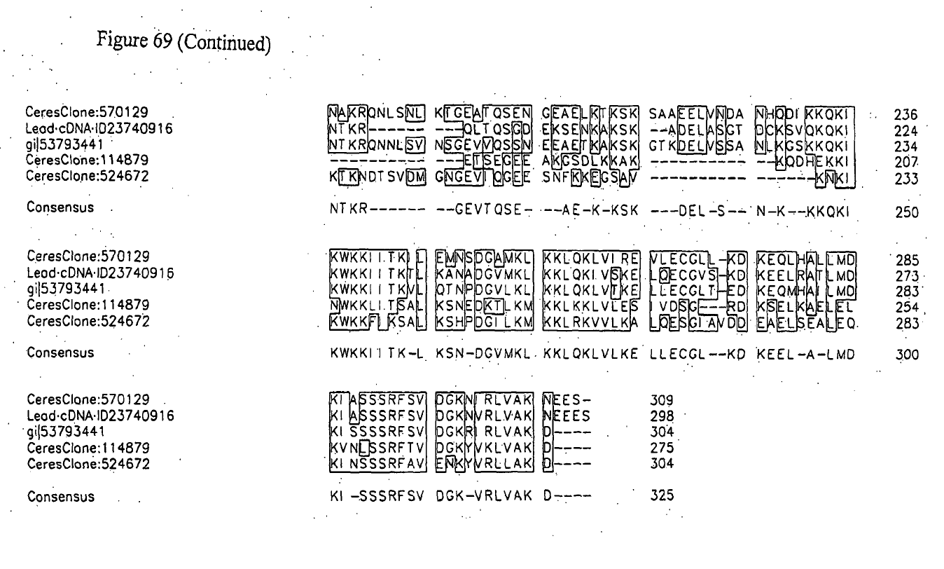

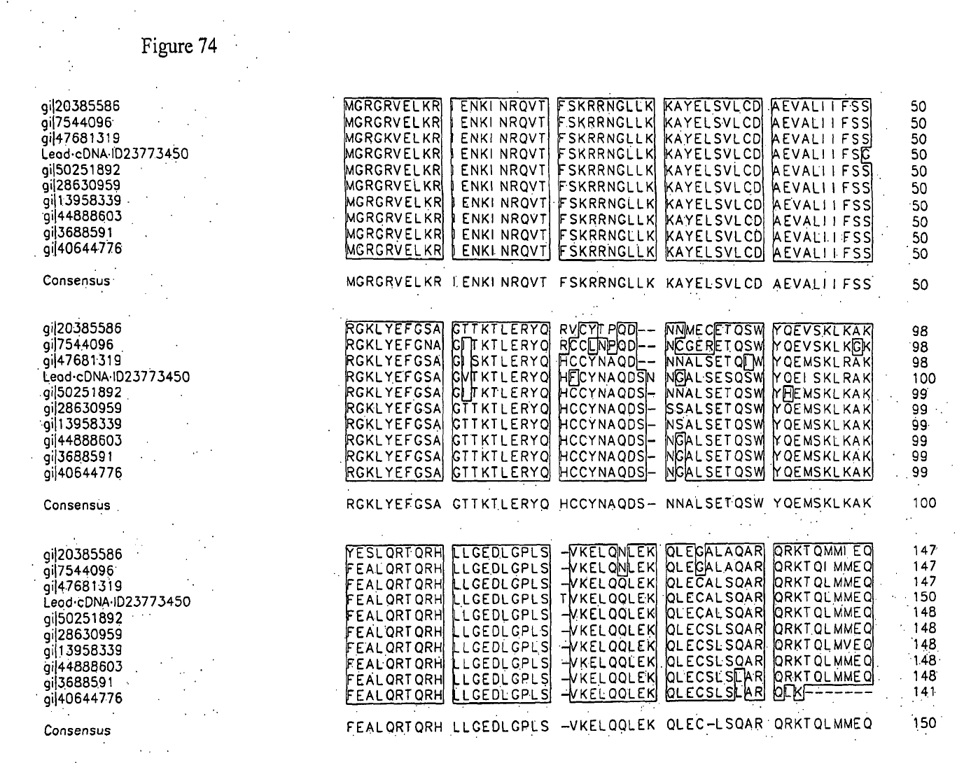

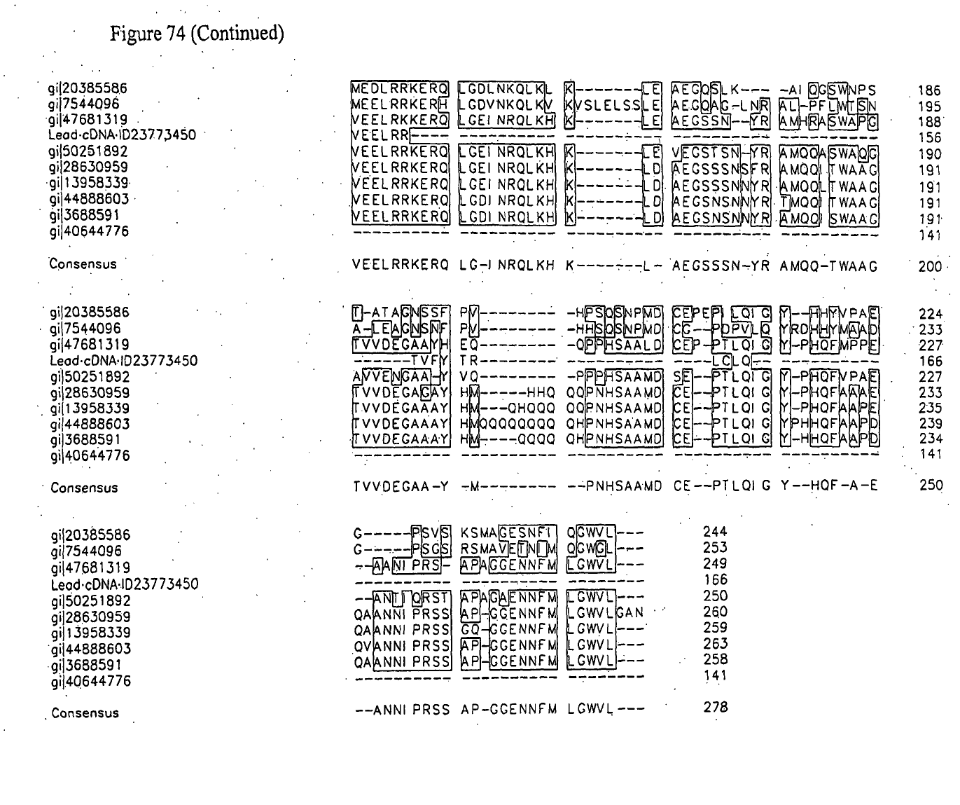

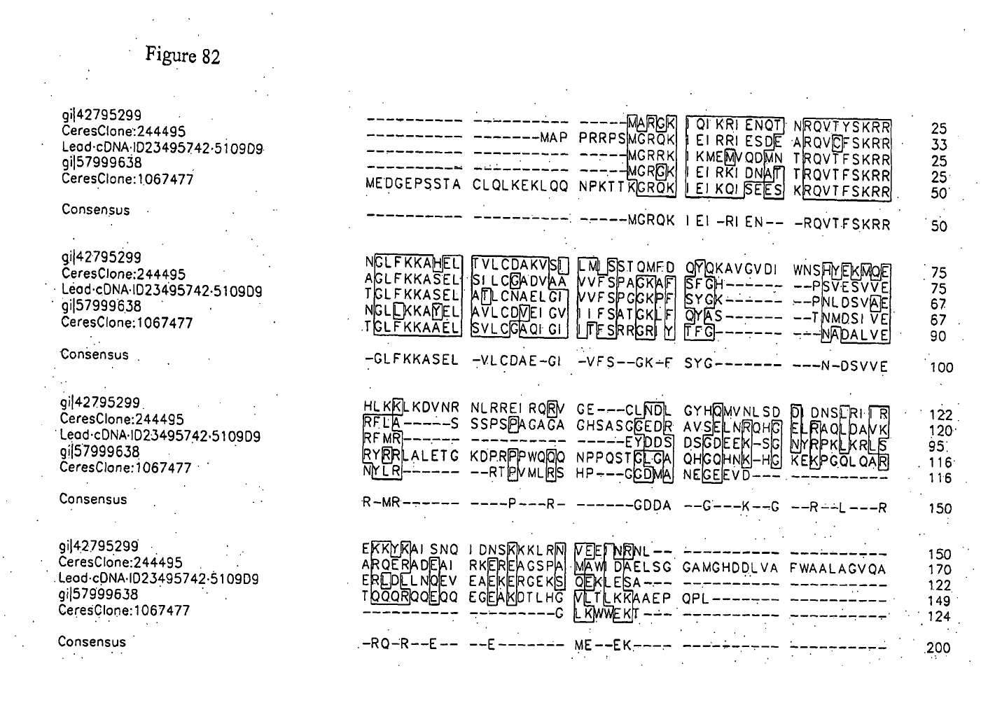

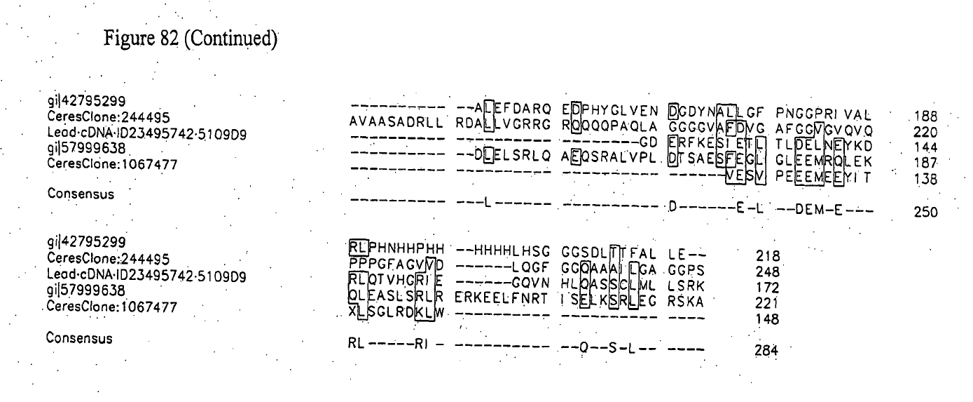

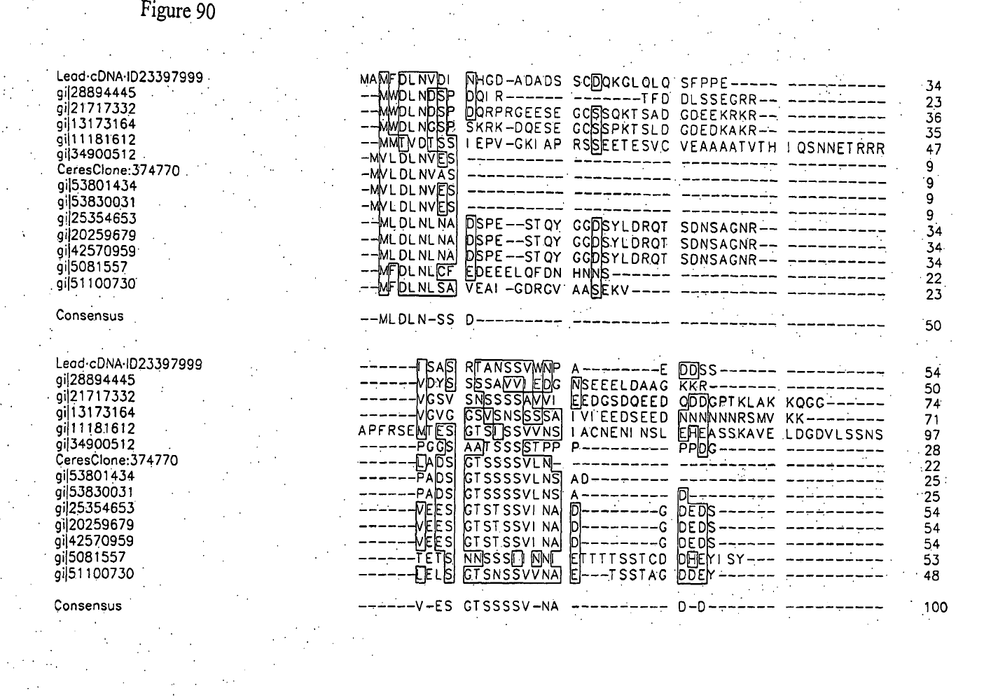

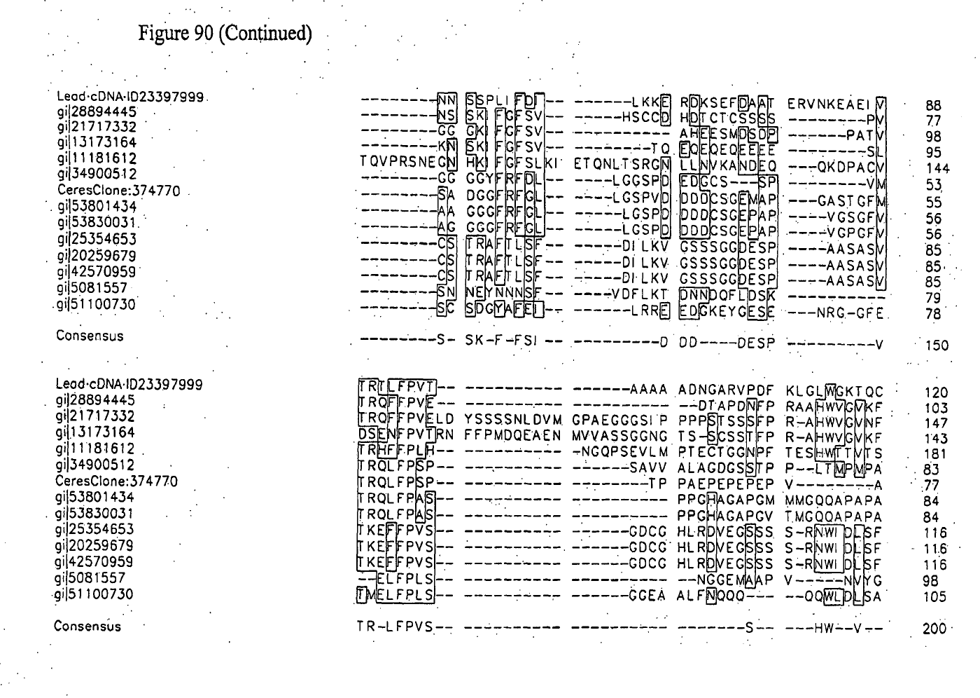

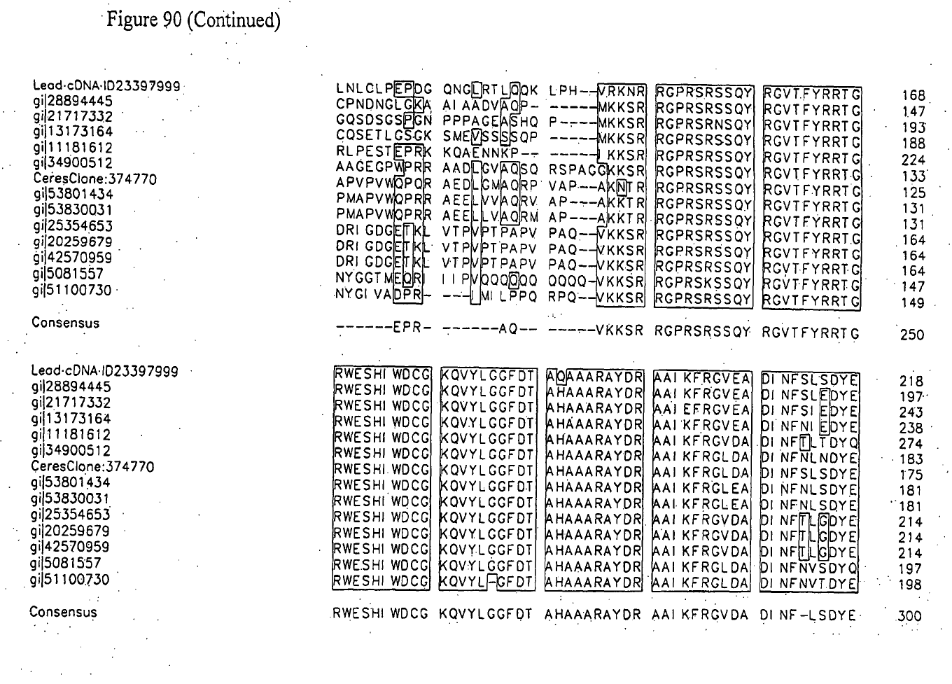

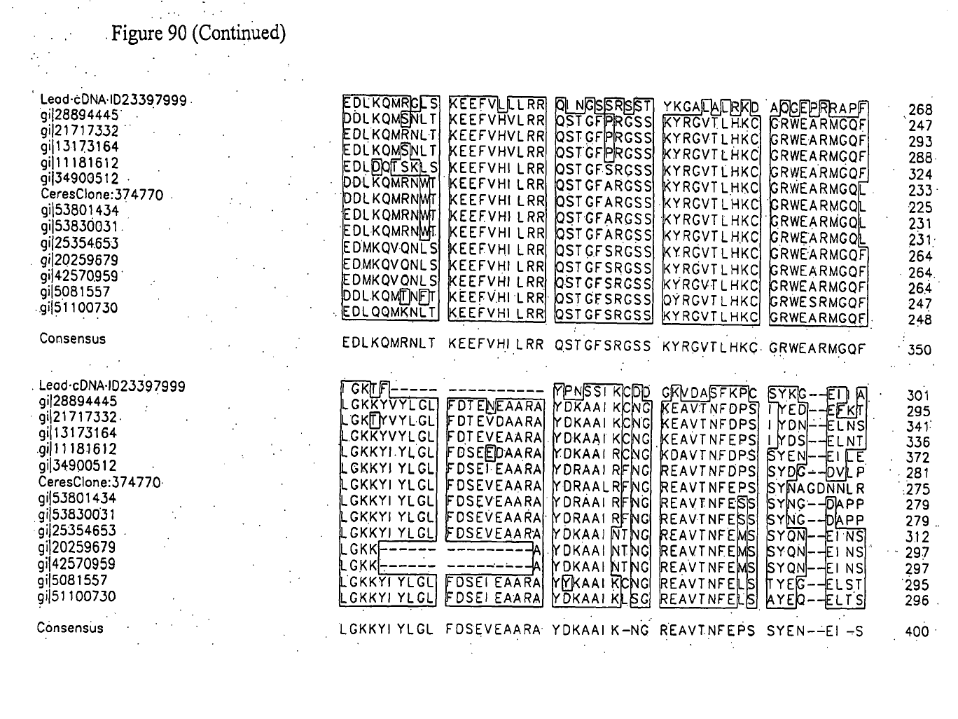

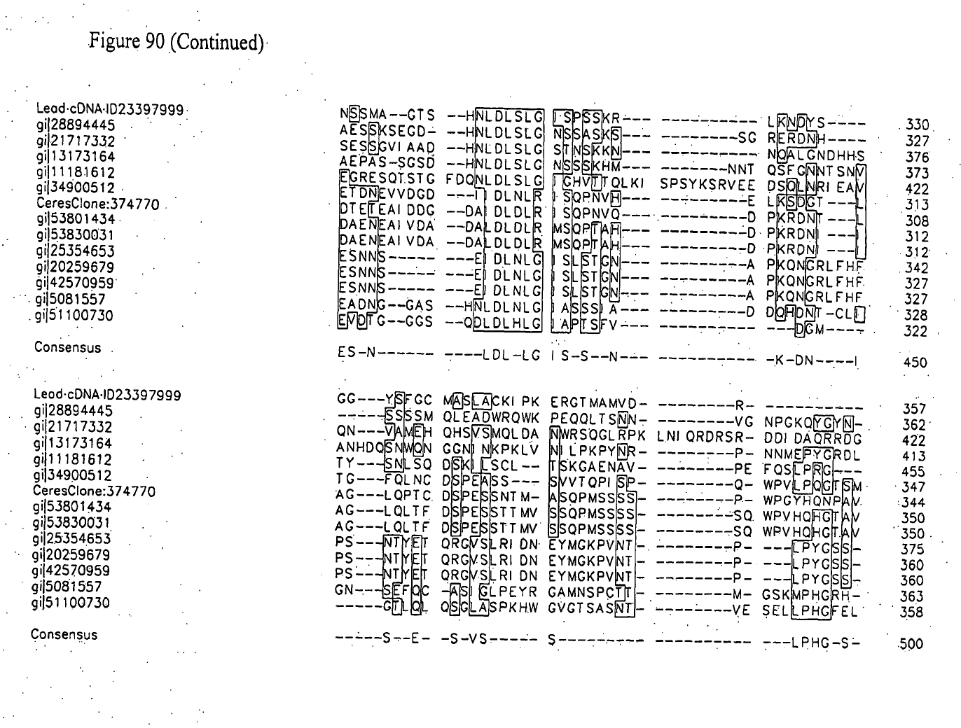

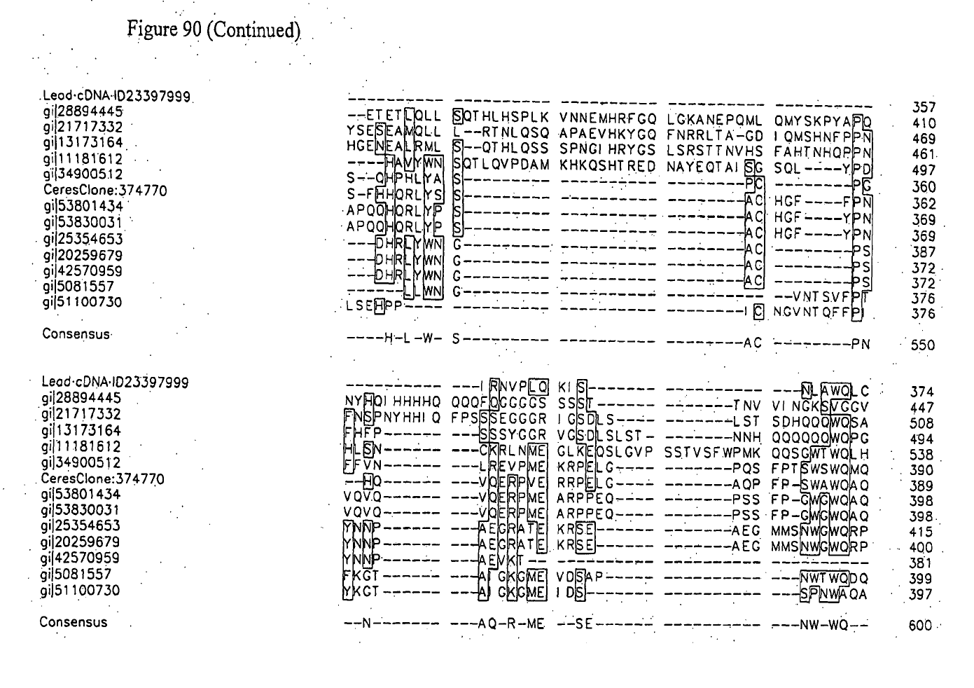

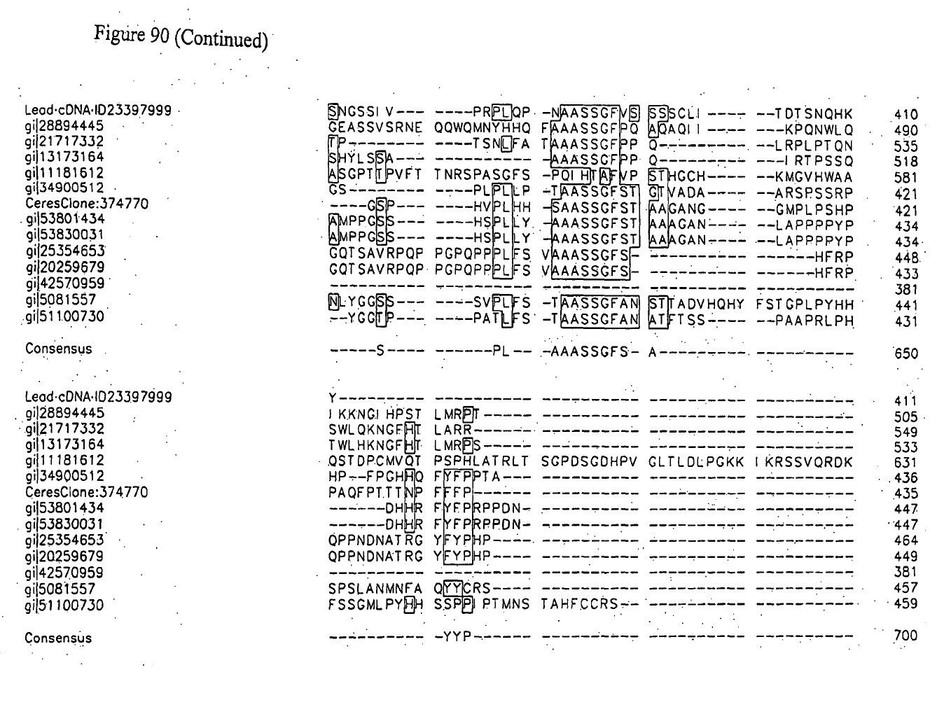

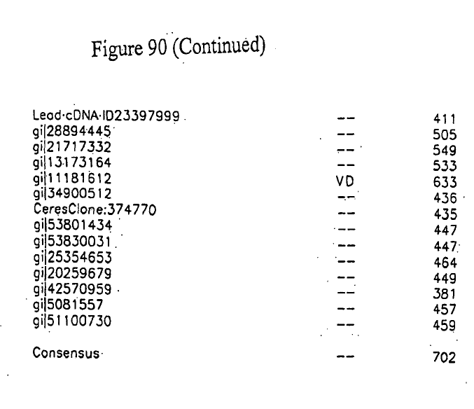

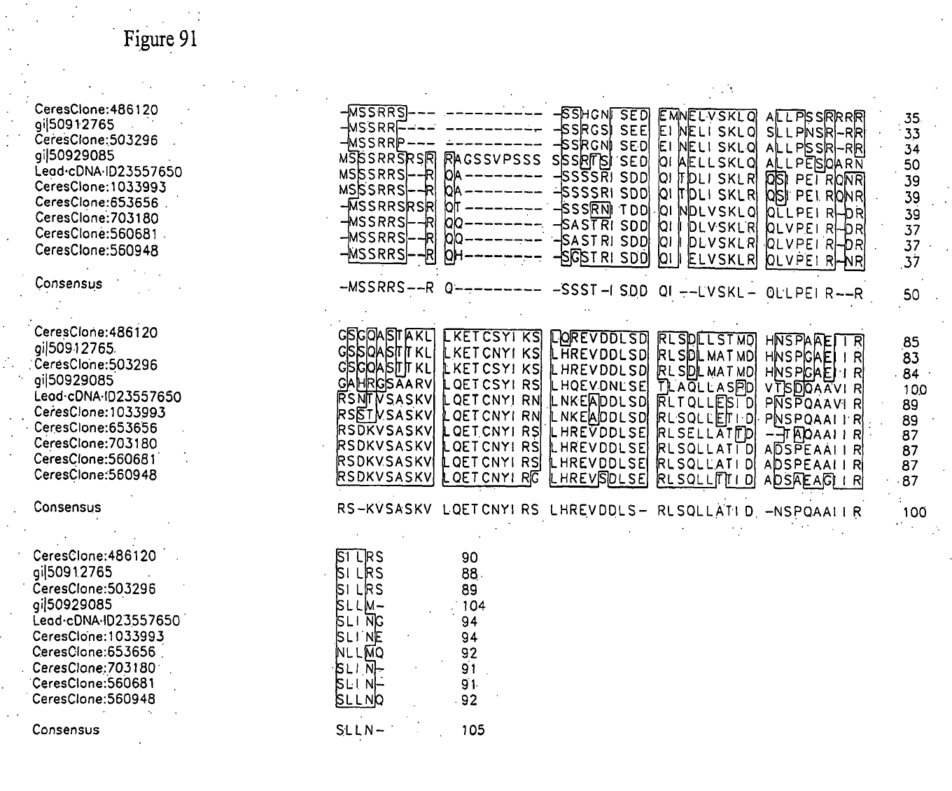

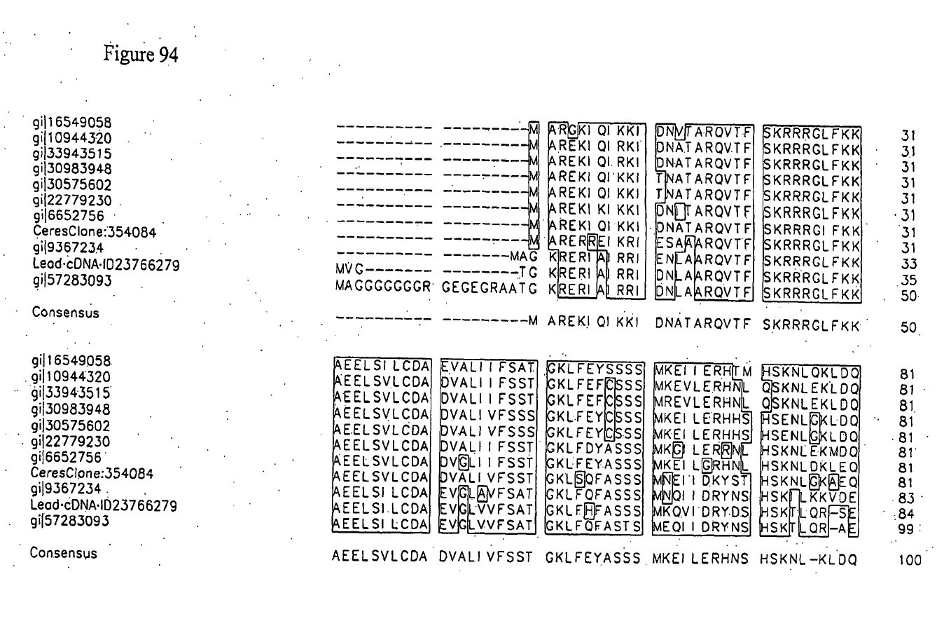

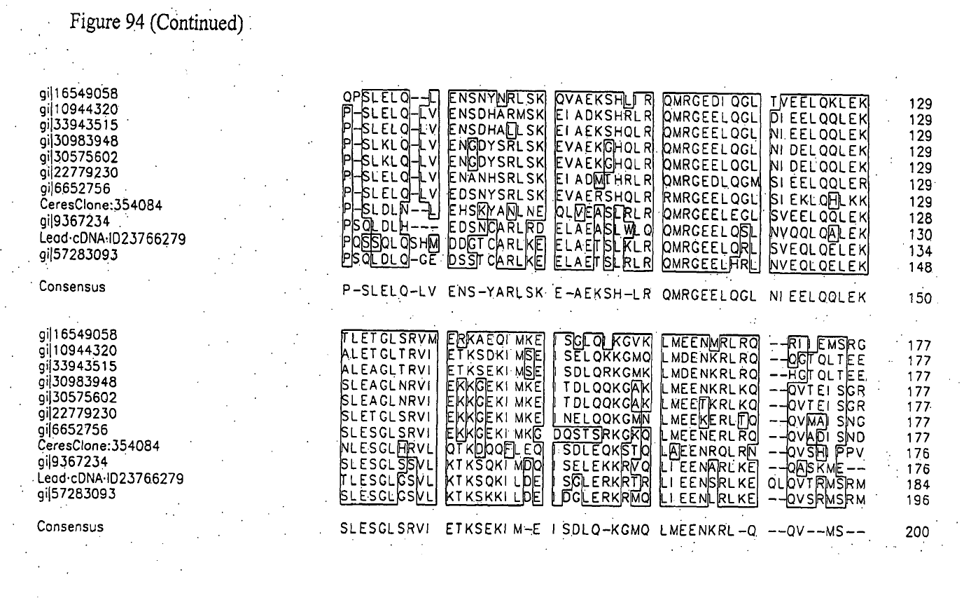

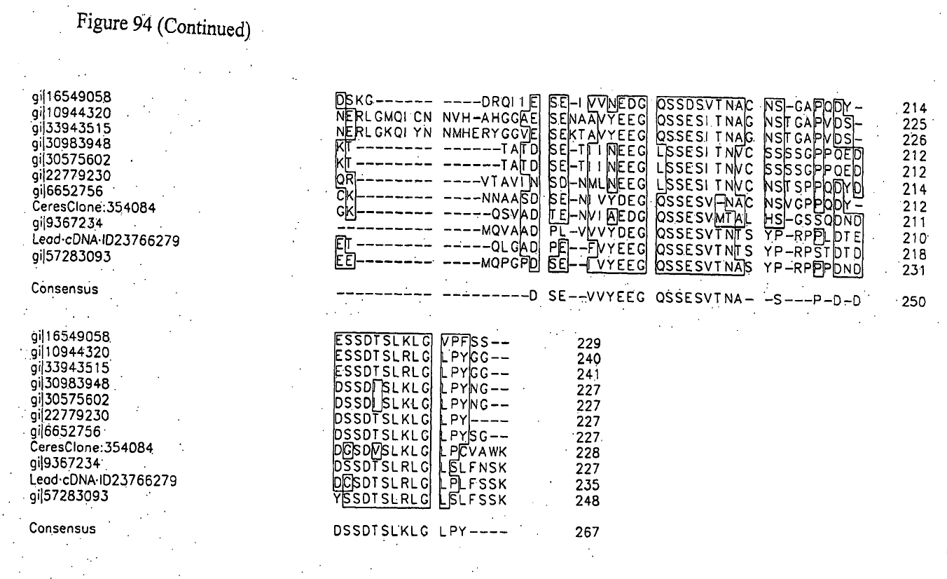

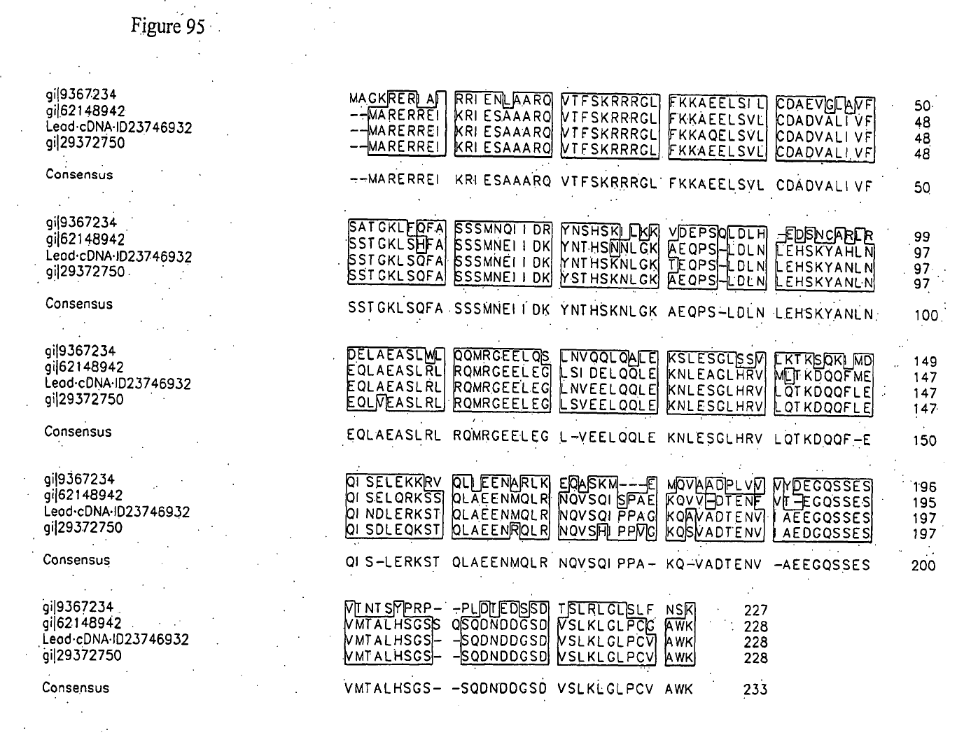

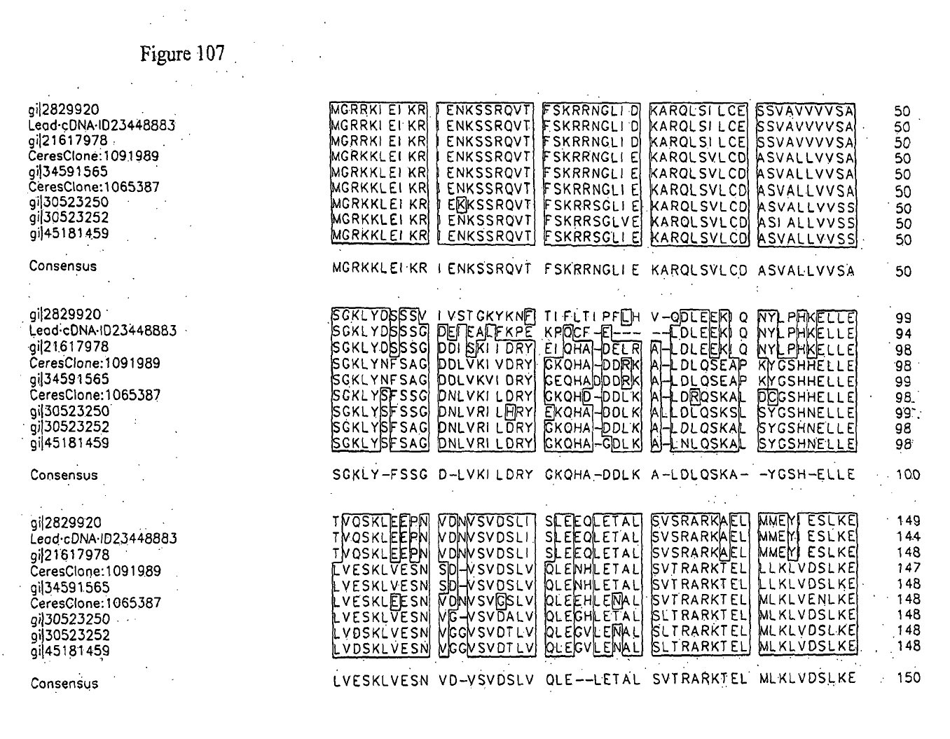

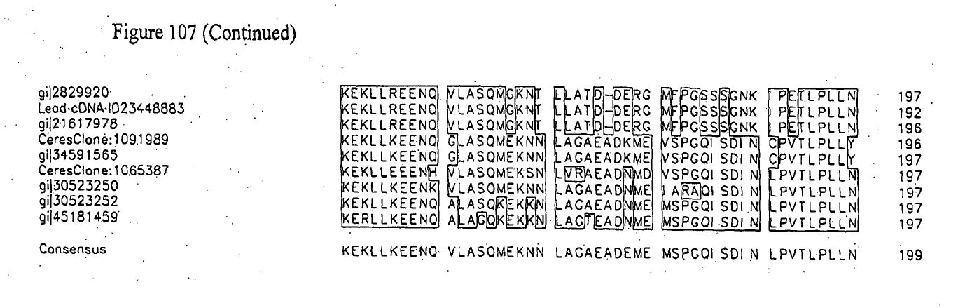

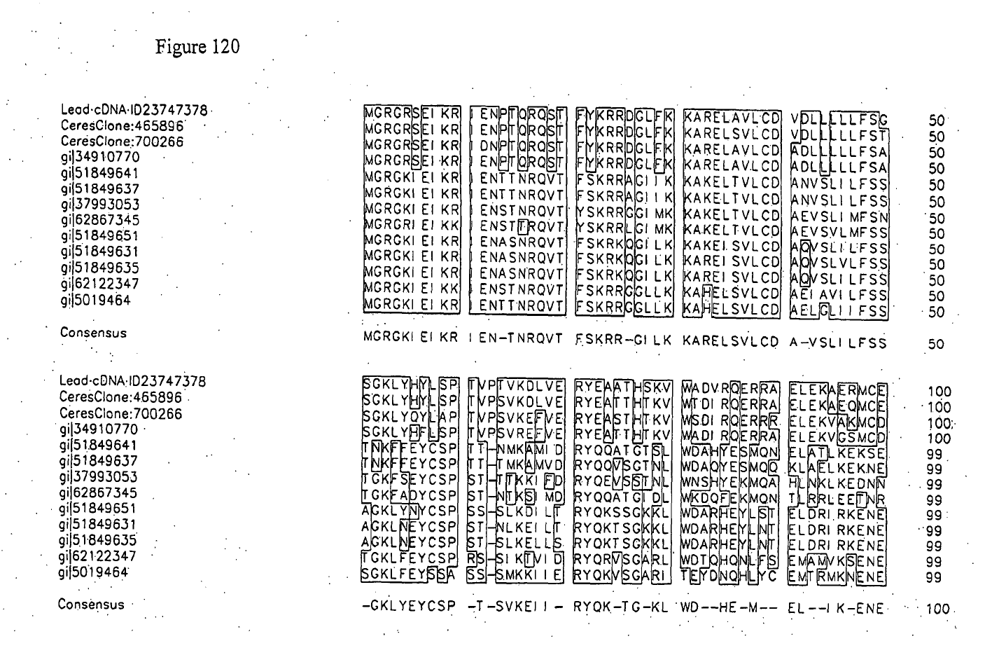

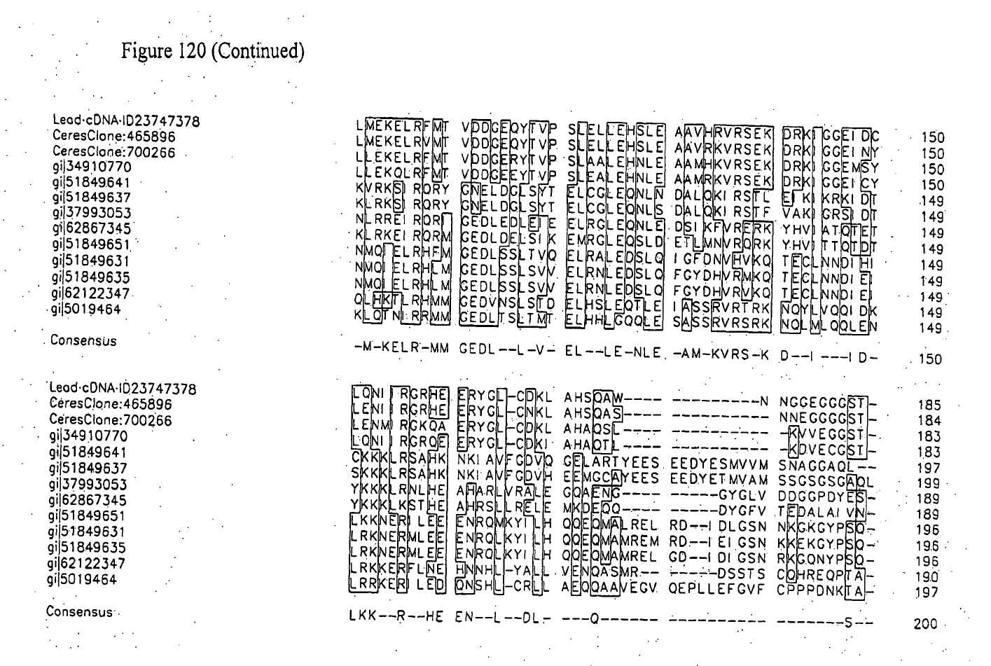

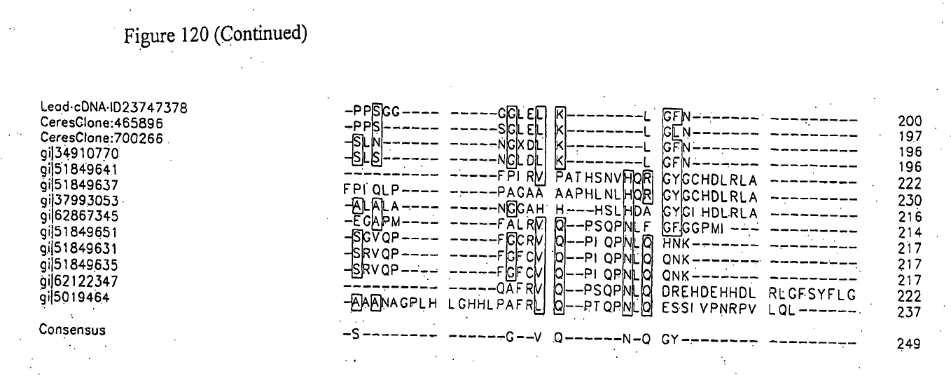

Amino acid sequences of homologs and/or orthologs of the polypeptide having the amino acid sequence set forth in SEQ ID NO:123, SEQ ID NO:698, SEQ ID NO:822, SEQ ID NO:216, SEQ ID NO:472, SEQ ID NO:532, SEQ ID NO:748, SEQ ID NO:889, SEQ ID NO:946, SEQ ID NO:964, SEQ ID NO:1102, and SEQ ID NO:1226 are provided in FIG. 5, FIG. 68, FIG. 82, FIG. 15, FIG. 44, FIG. 49, FIG. 74, FIG. 90, FIG. 94, FIG. 95, FIG. 107, and FIG. 120, respectively. Each of FIG. 5, FIG. 68, FIG. 82, FIG. 15, FIG. 44, FIG. 49, FIG. 74, FIG. 90, FIG. 94, FIG. 95, FIG. 107, and FIG. 120 also includes a consensus amino acid sequence determined by aligning homologous and/or orthologous amino acid sequences with the amino acid sequence set forth in SEQ ID NO:123, SEQ ID NO:698, SEQ ID NO:822, SEQ ID NO:216, SEQ ID NO:472, SEQ ID NO:532, SEQ ID NO:748, SEQ ID NO:889, SEQ ID NO:946, SEQ ID NO:964, SEQ ID NO:1102, or SEQ ID NO:1226, respectively.

For example, the alignment in FIG. 5 provides the amino acid sequences of cDNA ID 23522096 (5109D12; SEQ ID NO:123), gi|30523252 (SEQ ID NO:124), CeresClone:244495 (SEQ ID NO:125), gi|45181459 (SEQ ID NO:127), gi|52789958 (SEQ ID NO:128), gi|82313 (SEQ ID NO:129), gi|20219014 (SEQ ID NO:130), gi|6580941 (SEQ ID NO:131), gi|45268960 (SEQ ID NO:132), gi|55792842 (SEQ ID NO:133), gi|6580939 (SEQ ID NO:134), gi|46917358 (SEQ ID NO:135), gi|30523364 (SEQ ID NO:136), gi|55792848 (SEQ ID NO:137), gi|22091477 (SEQ ID NO:138), and gi|5031217 (SEQ ID NO:139). Other homologs and/or orthologs of SEQ ID NO:123 include Ceres CLONE ID no. 326824 (SEQ ID NO:126).

The alignment in FIG. 68 provides the amino acid sequences of cDNA ID 23494809 (5110G5; SEQ ID NO:698) and gi|32455231 (SEQ ID NO:699).

The alignment in FIG. 82 provides the amino acid sequences of cDNA ID 23495742 (5109D9; SEQ ID NO:822), gi|57999638 (SEQ ID NO:823), CeresClone:1067477 (SEQ ID NO:824), gi|42795299 (SEQ ID NO:825), and CeresClone:244495 (SEQ ID NO:826).

The alignment in FIG. 15 provides the amino acid sequences of cDNA ID 4984839 (5110G8; SEQ ID NO:216), gi|31580813 (SEQ ID NO:217) and gi|30523252 (SEQ ID NO:223). Other homologs and/or orthologs of SEQ ID NO:216 include Public GI no. 17933458 (SEQ ID NO:218), Public GI no. 17933450 (SEQ ID NO:219), Ceres CLONE ID no. 1065387 (SEQ ID NO:220), Public GI no. 17933456 (SEQ ID NO:221), and Ceres CLONE ID no. 1091989 (SEQ ID NO:222).

The alignment in FIG. 44 provides the amino acid sequences of cDNA ID 23783423 (SEQ ID NO:472), gi|9367307 (SEQ ID NO:473), gi|62510920 (SEQ ID NO:474), gi|28630957 (SEQ ID NO:475), gi|6175371 (SEQ ID NO:476), gi|33309864 (SEQ ID NO:477), gi|6467974 (SEQ ID NO:478), gi|1483232 (SEQ ID NO:479), CeresClone:510092 (SEQ ID NO:481), gi|29372764 (SEQ ID NO:482), gi|33355661 (SEQ ID NO:483), gi|30090030 (SEQ ID NO:484), gi|58423002 (SEQ ID NO:486), gi|33391153 (SEQ ID NO:487), and gi|39843110 (SEQ ID NO:488). Other homologs and/or orthologs of SEQ ID NO:472) include Public GI no. 38229935 (SEQ ID NO:480) and Public GI no. 32478105 (SEQ ID NO:485).

The alignment in FIG. 49 provides the amino acid sequences of cDNA ID 12680548 (SEQ ID NO:532), gi|62632894 (SEQ ID NO:533), CeresClone:1065387 (SEQ ID NO:534), gi|30523250 (SEQ ID NO:537), gi|30523252 (SEQ ID NO:538), gi|30523362 (SEQ ID NO:540), CeresClone:1091989 (SEQ ID NO:541), gi|30523360 (SEQ ID NO:543), and gi|30523366 (SEQ ID NO:546). Other homologs and/or orthologs of SEQ ID NO:532 include Public GI no. 17933450 (SEQ ID NO:535), Public GI no. 31580813 (SEQ ID NO:536), Ceres CLONE ID no. 963001 (SEQ ID NO:539), Public GI no. 17933456 (SEQ ID NO:542), Public GI no. 30523364 (SEQ ID NO:544), and Public GI no. 45181459 (SEQ ID NO:545).

The alignment in FIG. 74 provides the amino acid sequences of cDNA ID 23773450 (SEQ ID NO:748), gi|50251892 (SEQ ID NO:750), gi|44888603 (SEQ ID NO:751), gi|3688591 (SEQ ID NO:752), gi|13958339 (SEQ ID NO:753), gi|28630959 (SEQ ID NO:754), gi|40644776 (SEQ ID NO:755), gi|47681319 (SEQ ID NO:756), gi|7544096 (SEQ ID NO:757), and gi|20385586 (SEQ ID NO:758). Other homologs and/or orthologs of SEQ ID NO:748 include Public GI no. 7446515 (SEQ ID NO:749).

The alignment in FIG. 90 provides the amino acid sequences of cDNA ID 23556617 (SEQ ID NO:889), gi|23194453 (SEQ ID NO:890), gi|60100358 (SEQ ID NO:891), gi|3646326 (SEQ ID NO:892), CeresClone:1044034 (SEQ ID NO:893), gi|4103342 (SEQ ID NO:894), gi|20385590 (SEQ ID NO:896), gi|27763670 (SEQ ID NO:897), gi|57157565 (SEQ ID NO:898), gi|42794560 (SEQ ID NO:899), gi|29467048 (SEQ ID NO:900), gi|48727598 (SEQ ID NO:901), gi|21955182 (SEQ ID NO:902), and gi|1568513 (SEQ ID NO:903). Other homologs and/or orthologs of SEQ ID NO:889 include Public GI no. 2997615 (SEQ ID NO:895) and Public GI no. 1067169 (SEQ ID NO:904).

The alignment in FIG. 94 provides the amino acid sequences of cDNA ID 23766279 (SEQ ID NO:946), gi|57283093 (SEQ ID NO:947), gi|9367234 (SEQ ID NO:951), CeresClone:354084 (SEQ ID NO:952), gi|10944320 (SEQ ID NO:954), gi|33943515 (SEQ ID NO:956), gi|6652756 (SEQ ID NO:958), gi|16549058 (SEQ ID NO:959), gi|30983948 (SEQ ID NO:960), gi|30575602 (SEQ ID NO:961), and gi|22779230 (SEQ ID NO:962). Other homologs and/or orthologs include Public GI no. 33621119 (SEQ ID NO:948), Public GI no. 33621117 (SEQ ID NO:949), Public GI no. 9367232 (SEQ ID NO:950), Public GI no. 29372750 (SEQ ID NO:953), Public GI no. 51968624 (SEQ ID NO:955), and Public GI no. 33943513 (SEQ ID NO:957).

The alignment in FIG. 95 provides the amino acid sequences of cDNA ID 23746932 (SEQ ID NO:964), gi|29372750 (SEQ ID NO:965), gi|62148942 (SEQ ID NO:966), and gi|9367234 (SEQ ID NO:971). Other homologs and/or orthologs of SEQ ID NO:964 include Public GI no. 51091146 (SEQ ID NO:967), Ceres CLONE ID no. 300498 (SEQ ID NO:968), Public GI no. 29372754 (SEQ ID NO:969), and Ceres CLONE ID no. 277135 (SEQ ID NO:970).

The alignment in FIG. 107 provides the amino acid sequences of cDNA ID 23448883 (SEQ ID NO:1102), gi|21617978 (SEQ ID NO:1104), gi|2829920 (SEQ ID NO:1105), CeresClone:1065387 (SEQ ID NO:1107), CeresClone:1091989 (SEQ ID NO:1110), gi|34591565 (SEQ ID NO:1112), gi|30523250 (SEQ ID NO:1113), gi|30523252 (SEQ ID NO:1114), and gi|45181459 (SEQ ID NO:1115). Other homologs and/or orthologs of SEQ ID NO:1102 include Ceres CLONE ID no. 92459 (SEQ ID NO:1103), Public GI no. 31580813 (SEQ ID NO:1106), Public GI no. 17933450 (SEQ ID NO:1108), Public GI no. 17933458 (SEQ ID NO:1109), Public GI no. 17933456 (SEQ ID NO:1111), Ceres CLONE ID no. 963001 (SEQ ID NO:1116), and Public GI no. 30523362 (SEQ ID NO:1117).

The alignment in FIG. 120 provides the amino acid sequences of cDNA ID 23747378 (SEQ ID NO:1226), gi|62122347 (SEQ ID NO:1227), gi|5019464 (SEQ ID NO:1228), gi|51849631 (SEQ ID NO:1229), gi|51849641 (SEQ ID NO:1230), gi|51849637 (SEQ ID NO:1231), CeresClone:700266 (SEQ ID NO:1232), CeresClone:465896 (SEQ ID NO:1233), gi|37993053 (SEQ ID NO:1235), gi|34910770 (SEQ ID NO:1237), gi|51849651 (SEQ ID NO:1238), gi|51849635 (SEQ ID NO:1240), and gi|62867345 (SEQ ID NO:1241). Other homologs and/or orthologs of SEQ ID NO:1226 include Ceres CLONE ID no. 302467 (SEQ ID NO:1234), Public GI no. 37993051 (SEQ ID NO:1236), and Public GI no. 51849649 (SEQ ID NO:1239).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:124-139, SEQ ID NO:699, SEQ ID NOs:823-826, SEQ ID NOs:217-223, SEQ ID NOs:473-488, SEQ ID NOs:533-546, SEQ ID NOs:749-758, SEQ ID NOs:890-904, SEQ ID NOs:947-962, SEQ ID NOs:965-971, SEQ ID NOs:1103-1117, SEQ ID NOs:1227-1241, or the consensus sequence set forth in FIG. 5, FIG. 68, FIG. 82, FIG. 15, FIG. 44, FIG. 49, FIG. 74, FIG. 90, FIG. 94, FIG. 95, FIG. 107, or FIG. 120.

A regulatory protein can contain an AP2 domain characteristic of polypeptides belonging to the AP2/EREBP family of plant transcription factor polypeptides. AP2 (APETALA2) and EREBPs (ethylene-responsive element binding proteins) are prototypic members of a family of transcription factors unique to plants, whose distinguishing characteristic is that they contain the so-called AP2 DNA binding domain. AP2/EREBP genes form a large multigene family encoding polypeptides that play a variety of roles throughout the plant life cycle: from being key regulators of several developmental processes, such as floral organ identity determination and control of leaf epidermal cell identity, to forming part of the mechanisms used by plants to respond to various types of biotic and environmental stress. SEQ ID NO:80, SEQ ID NO:246, SEQ ID NO:264, SEQ ID NO:350, SEQ ID NO:874, SEQ ID NO:992, SEQ ID NO:1068, SEQ ID NO:1323, SEQ ID NO:1340, SEQ ID NO:1351, and SEQ ID NO:1376 set forth the amino acid sequences of DNA clones, identified herein as cDNA ID 23798983 (SEQ ID NO:79), cDNA ID 23411827 (SEQ ID NO:245), cDNA ID 23367111 (SEQ ID NO:263), cDNA ID 23419606 (SEQ ID NO:349), cDNA ID 23397999 (SEQ ID NO:873), cDNA ID 23416775 (SEQ ID NO:991), cDNA ID 23471864 (SEQ ID NO:1067), cDNA ID 23420963 (SEQ ID NO:1322), cDNA ID 23373703 (SEQ ID NO:1339), cDNA ID 23557531 (SEQ ID NO:1350), and cDNA ID 23394987 (SEQ ID NO:1375), respectively, that are predicted to encode AP2 domain-containing transcription factor polypeptides.

In some cases, a regulatory protein can contain an AP2 domain and a B3 DNA binding domain characteristic of a family of plant transcription factors with various roles in development. A B3 DNA binding domain is found in VP1/AB13 transcription factors.

Some proteins, such as RAV1, also have an AP2 DNA binding domain. SEQ ID NO:1358 sets forth the amino acid sequence of a DNA clone, identified herein as cDNA ID 23402435, that is predicted to encode a polypeptide having an AP2 and a B3 DNA binding domain.

A regulatory protein can comprise the amino acid sequence set forth in SEQ ID NO:80, SEQ ID NO:246, SEQ ID NO:264, SEQ ID NO:350, SEQ ID NO:874, SEQ ID NO:992, SEQ ID NO:1068, SEQ ID NO:1323, SEQ ID NO:1340, SEQ ID NO:1351, SEQ ID NO:1376, or SEQ ID NO:1358. Alternatively, a regulatory protein can be a homolog, ortholog, or variant of the polypeptide having the amino acid sequence set forth in SEQ ID NO:80, SEQ ID NO:246, SEQ ID NO:264, SEQ ID NO:350, SEQ ID NO:874, SEQ ID NO:992, SEQ ID NO:1068, SEQ ID NO:1323, SEQ ID NO:1340, SEQ ID NO:1351, SEQ ID NO:1376, or SEQ ID NO:1358. For example, a regulatory protein can have an amino acid sequence with at least 40% sequence identity, e.g., 40%, 41%, 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:80, SEQ ID NO:246, SEQ ID NO:264, SEQ ID NO:350, SEQ ID NO:874, SEQ ID NO:992, SEQ ID NO:1068, SEQ ID NO:1323, SEQ ID NO:1340, SEQ ID NO:1351, SEQ ID NO:1376, or SEQ ID NO:1358.

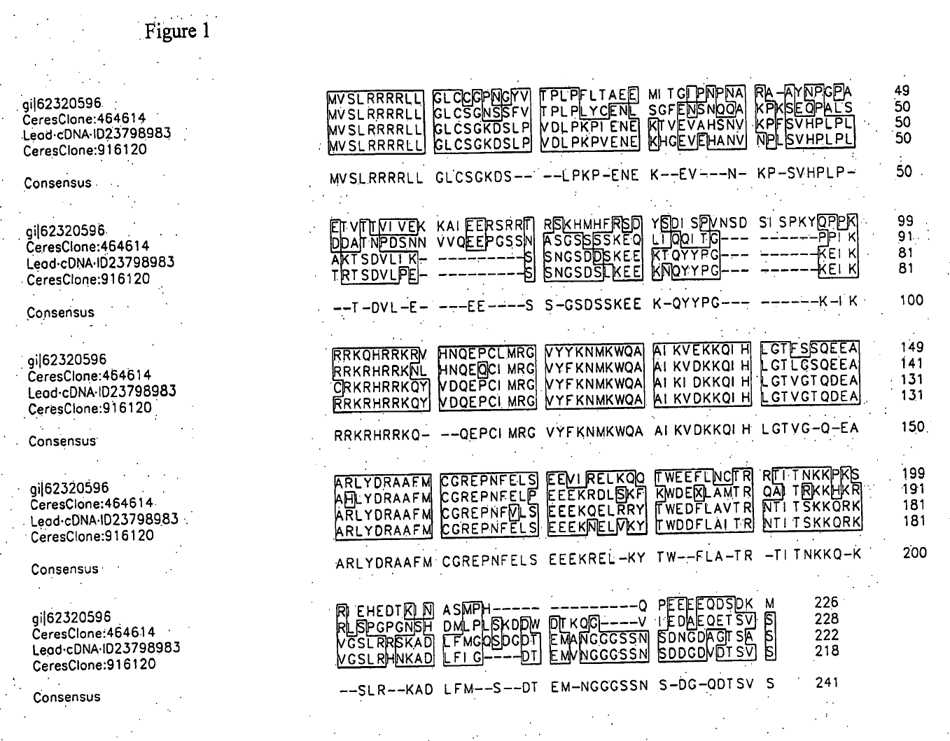

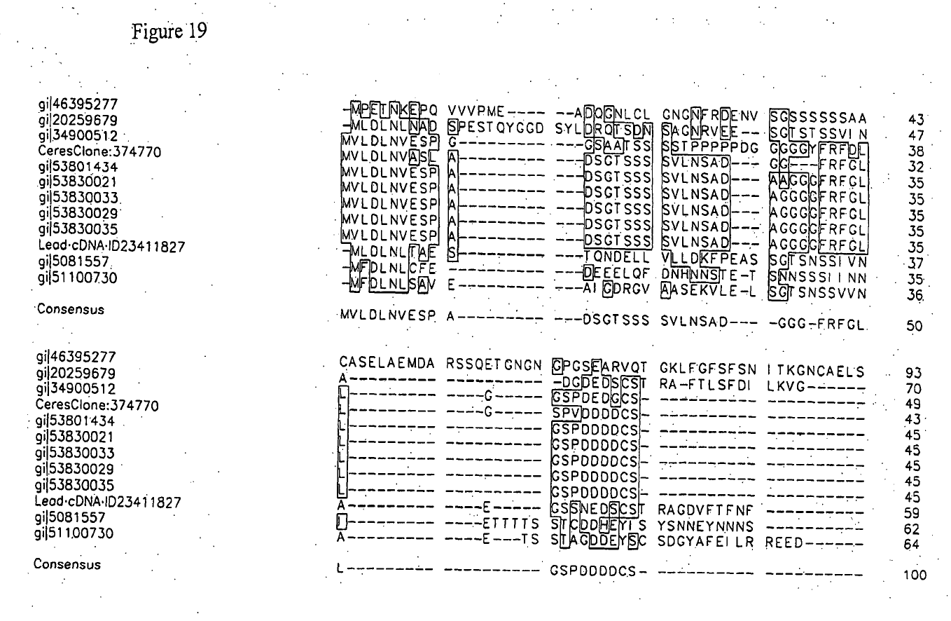

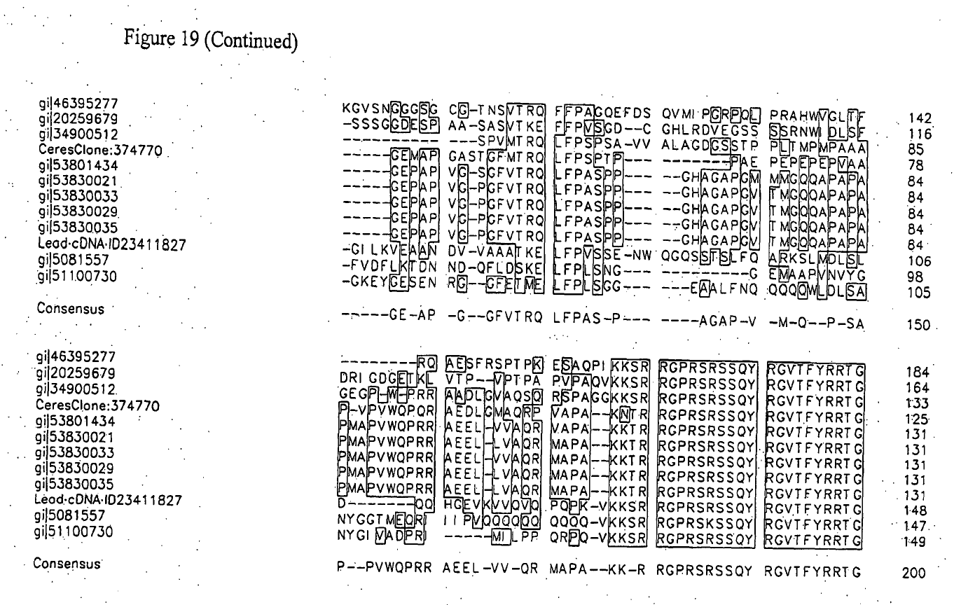

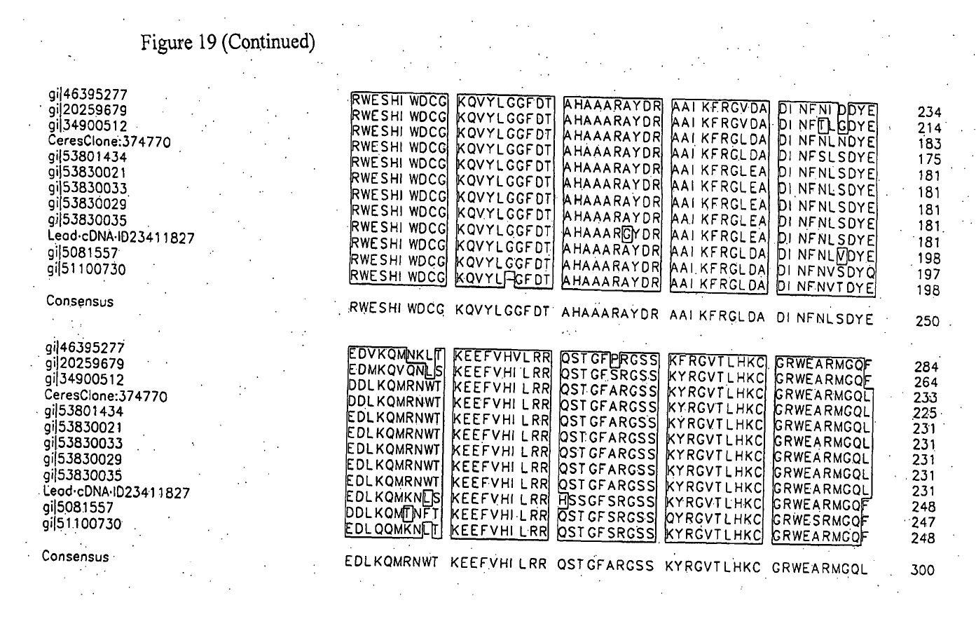

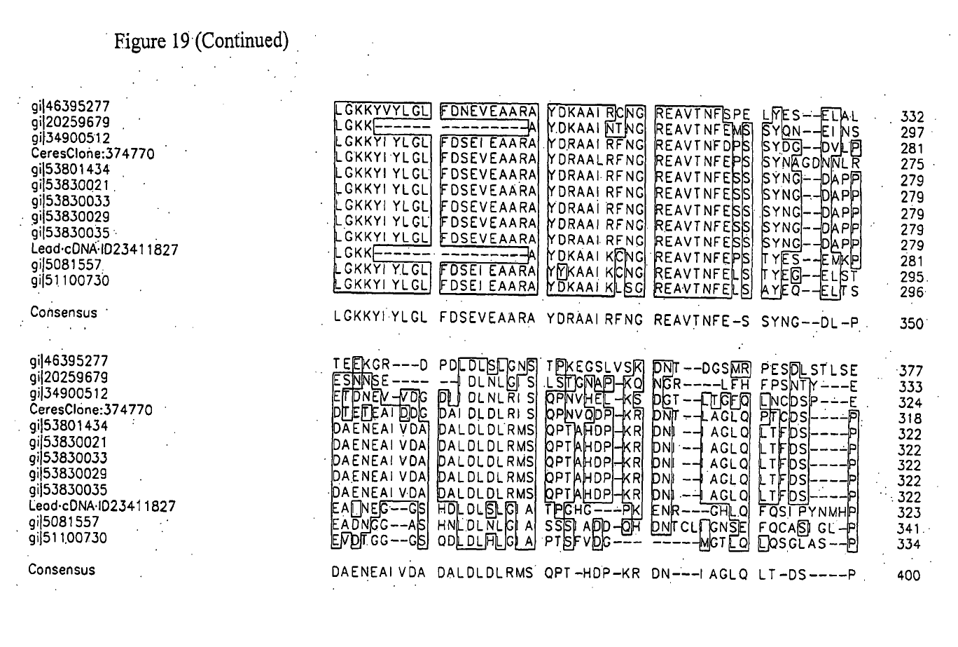

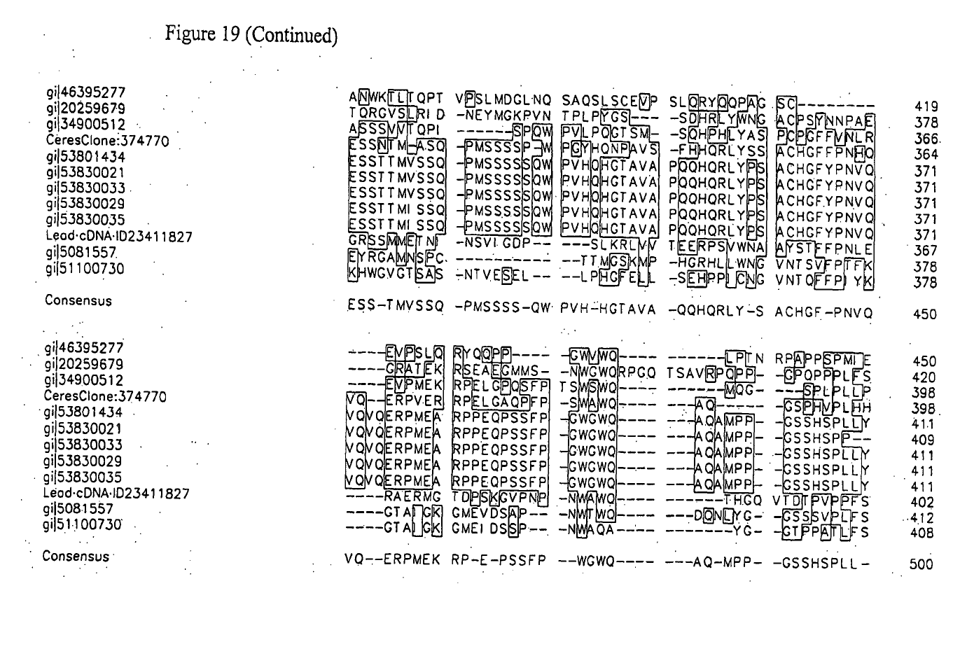

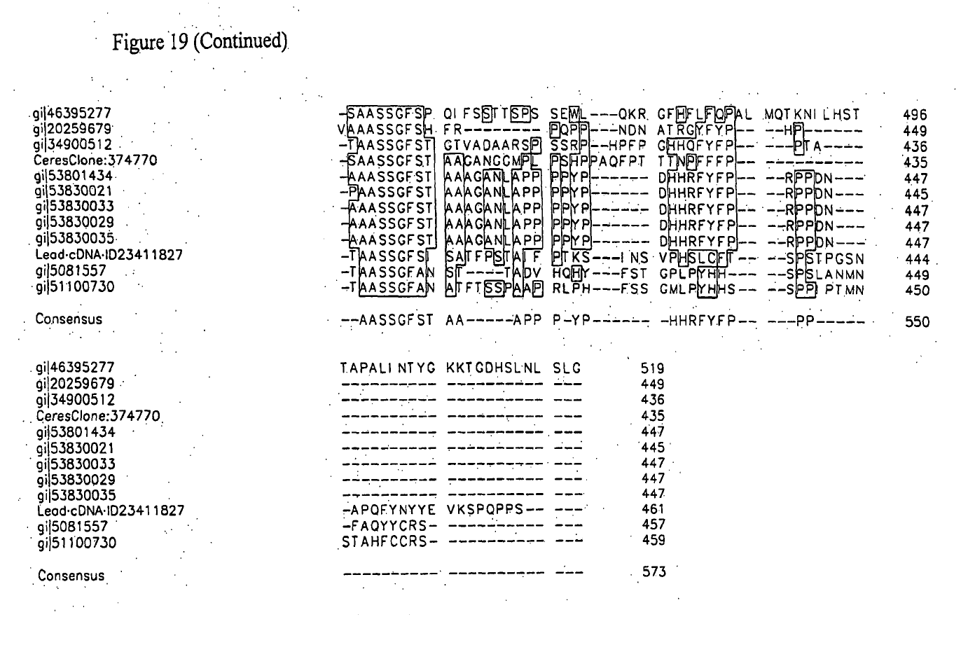

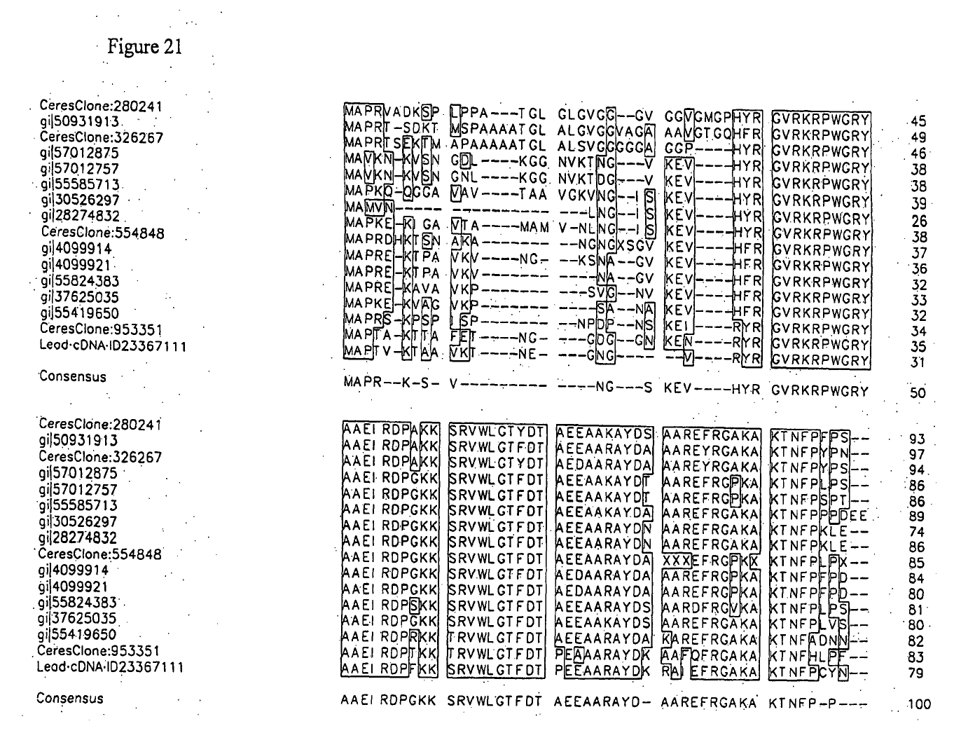

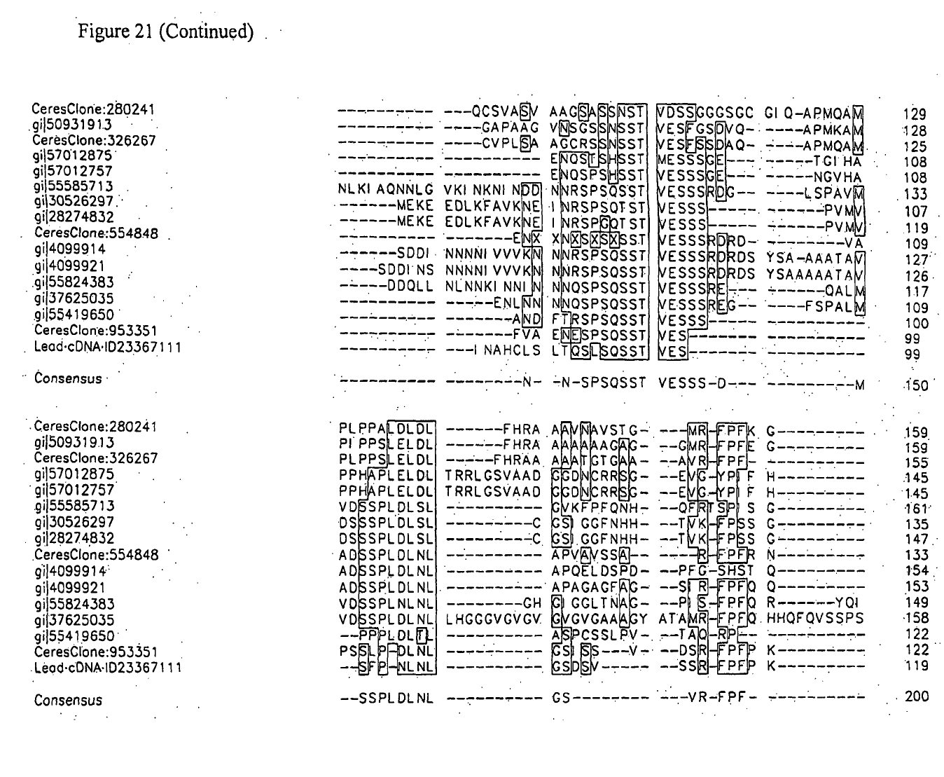

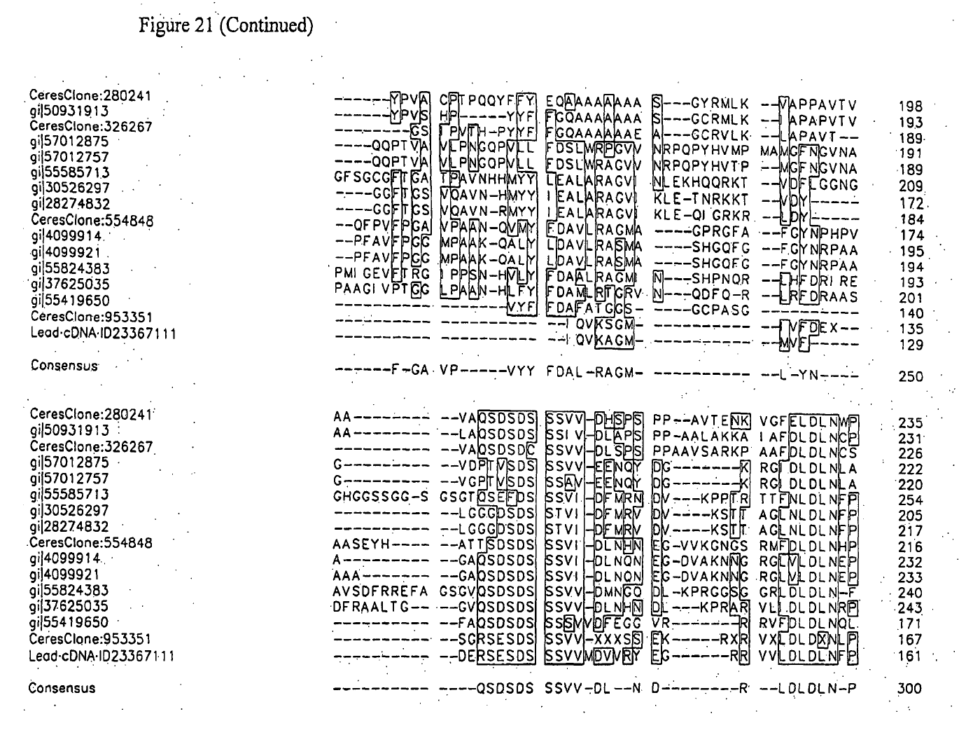

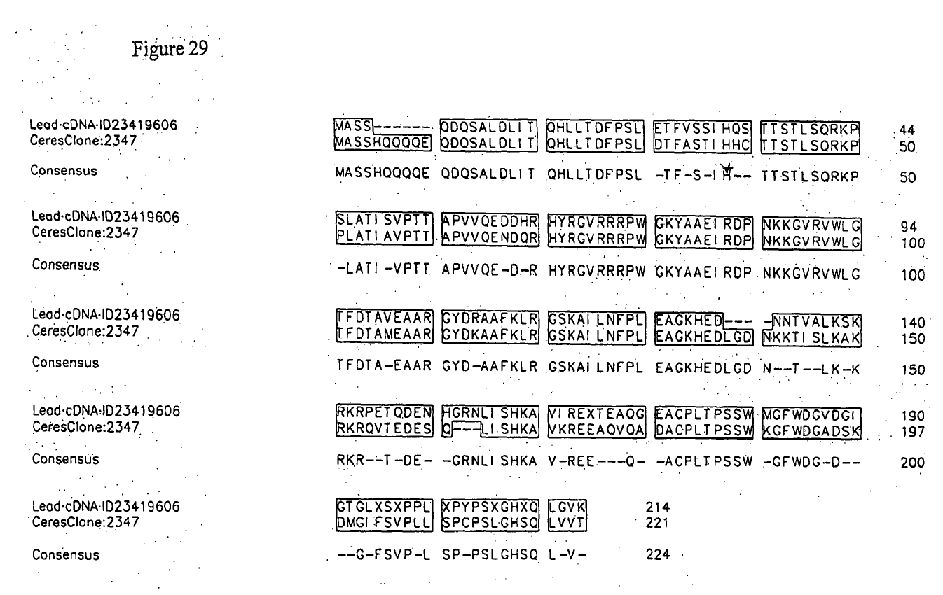

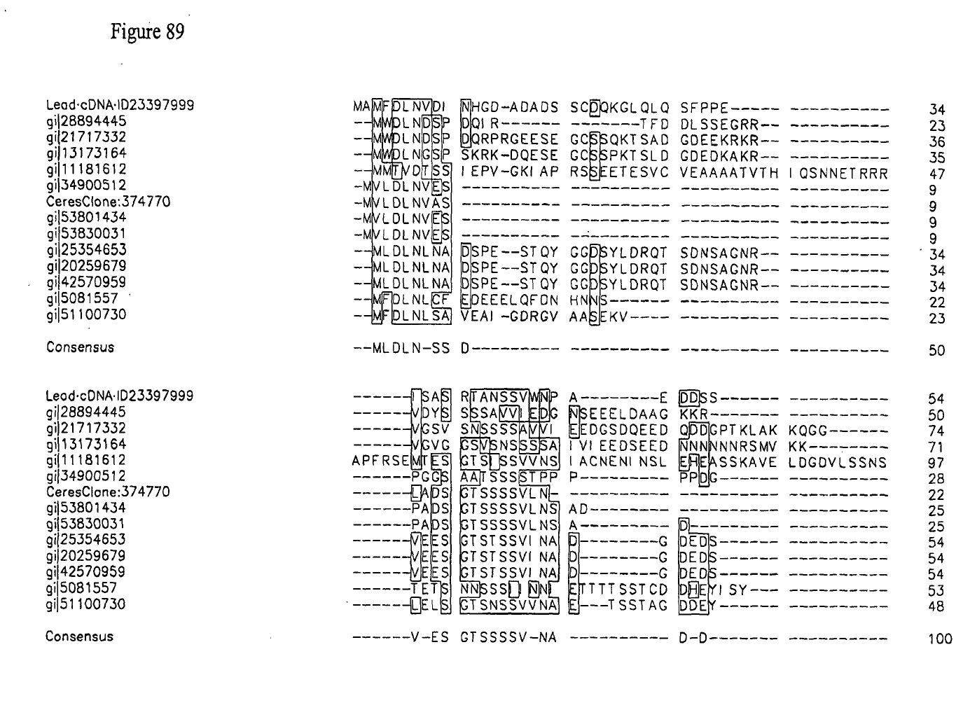

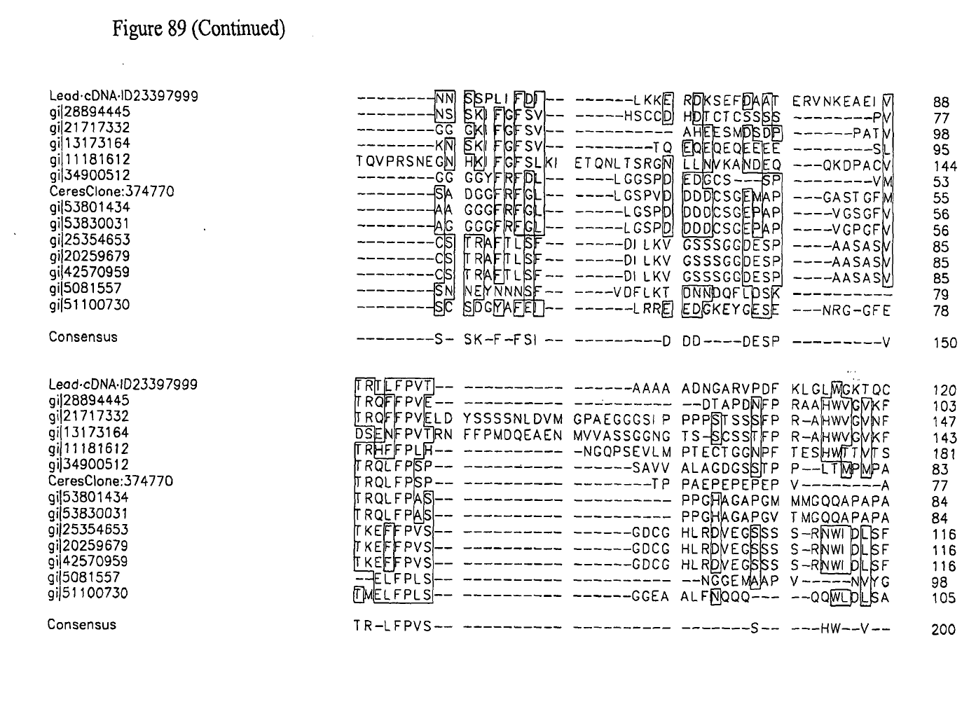

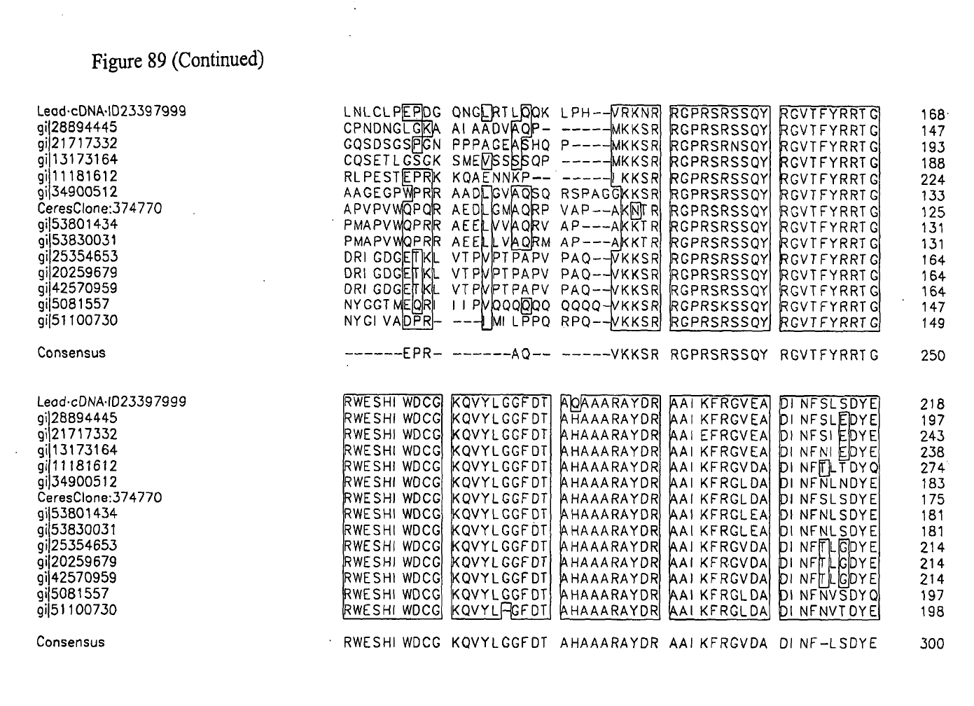

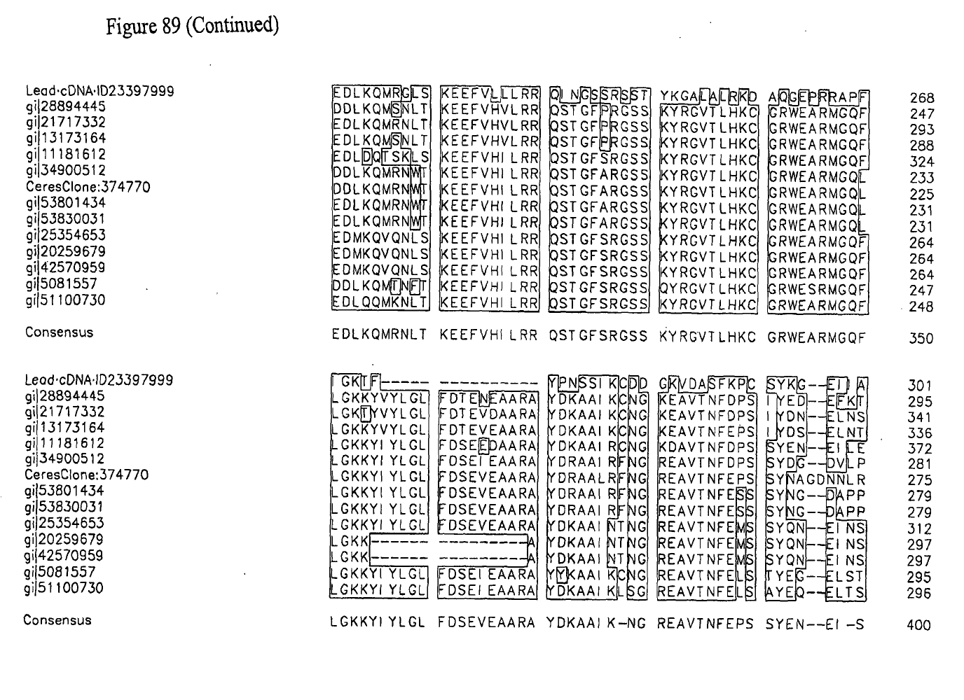

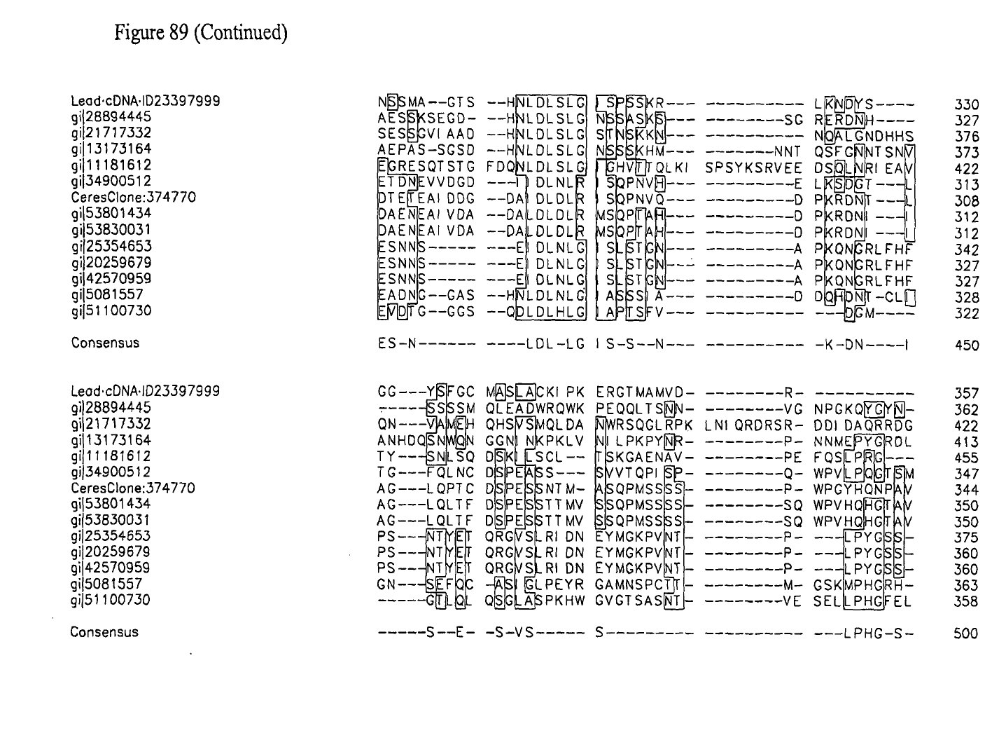

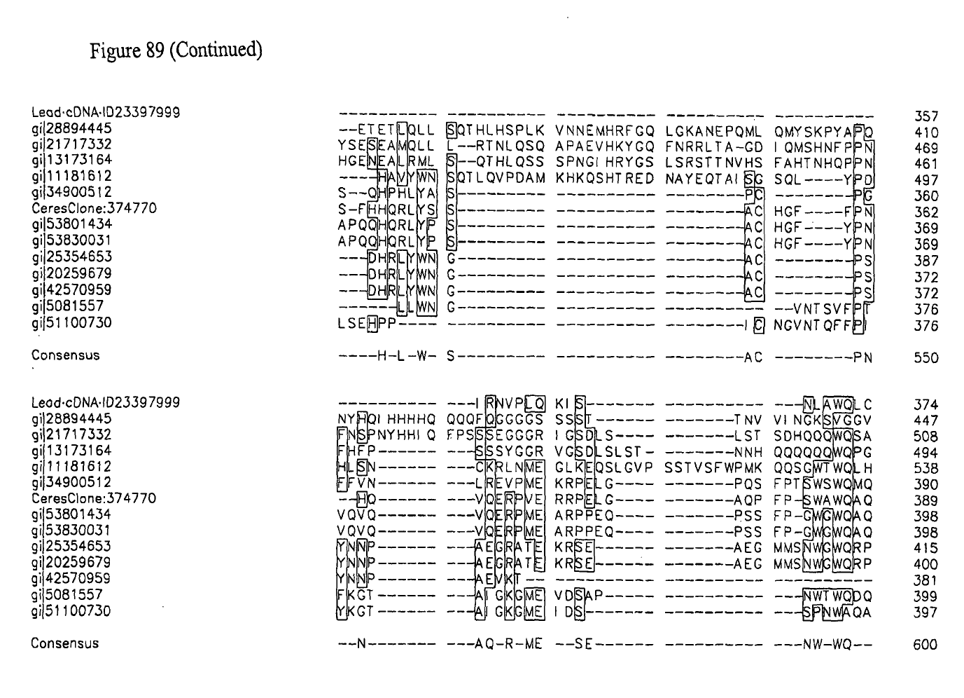

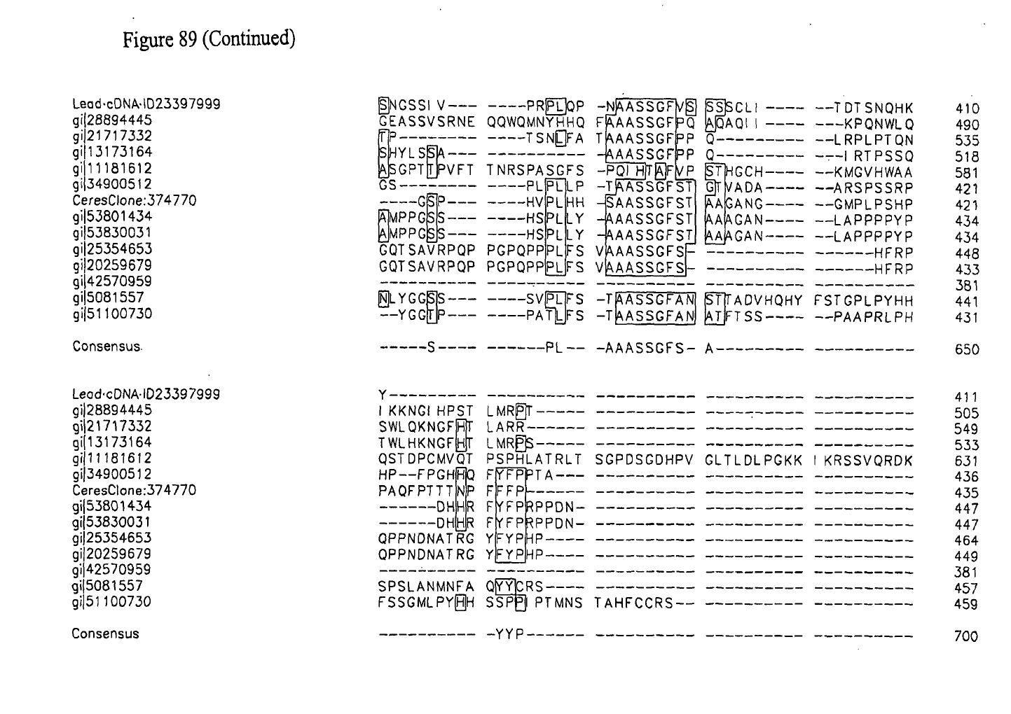

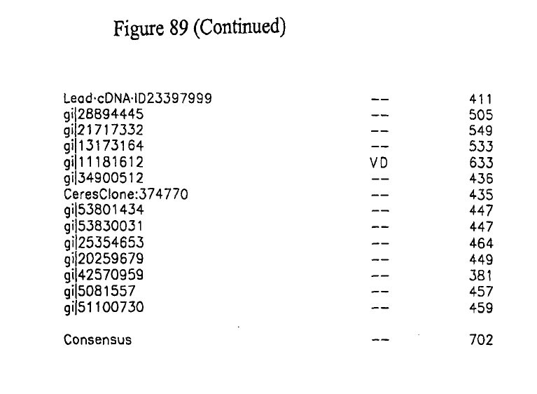

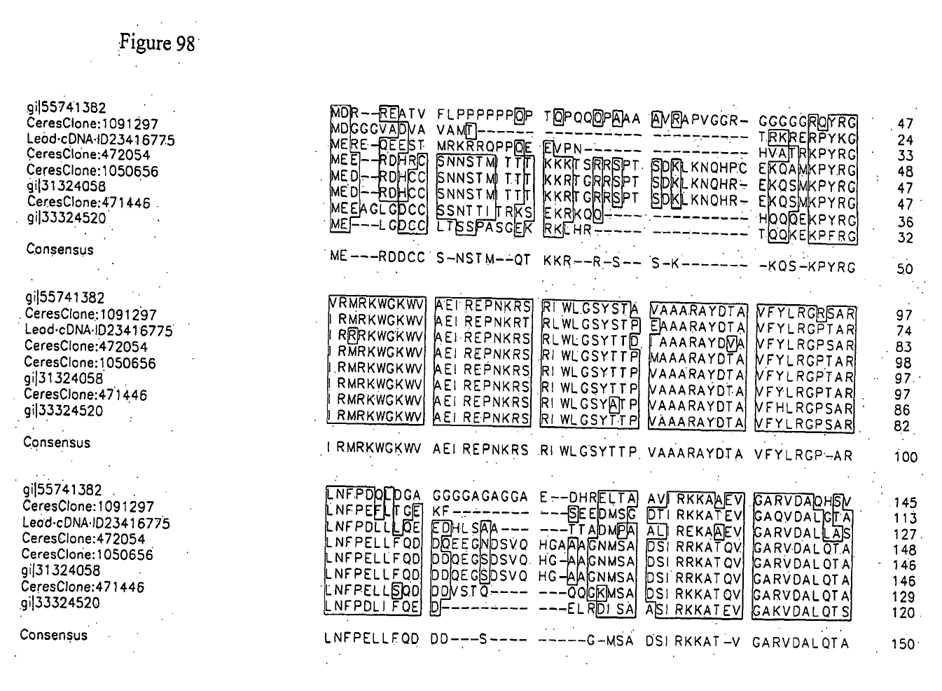

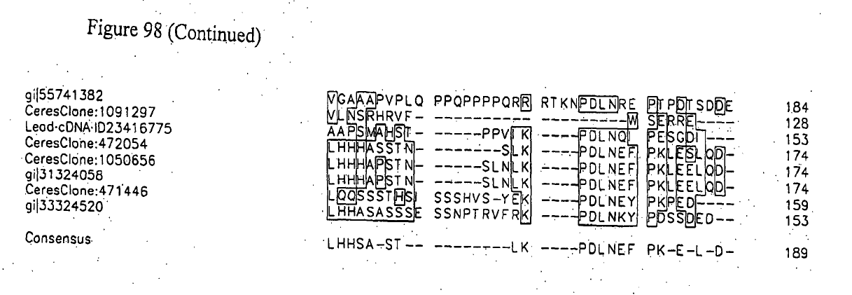

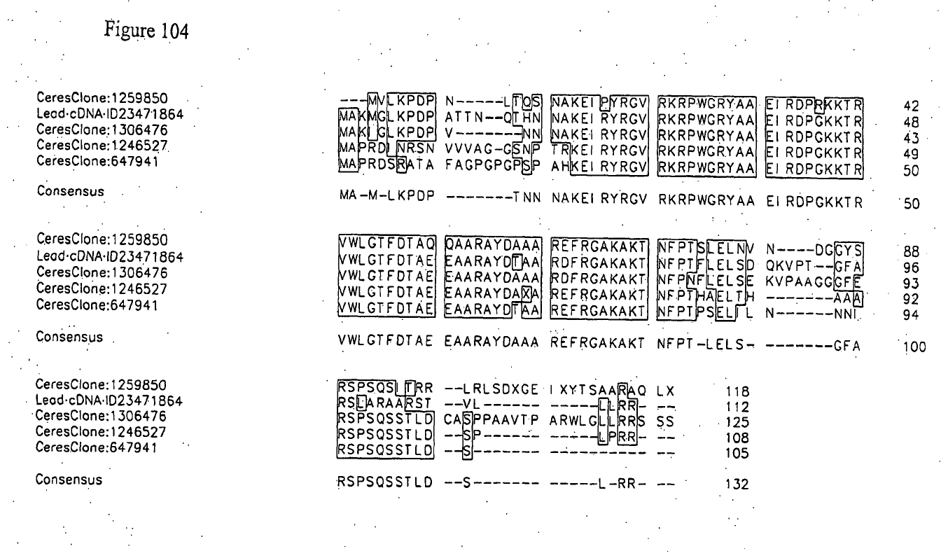

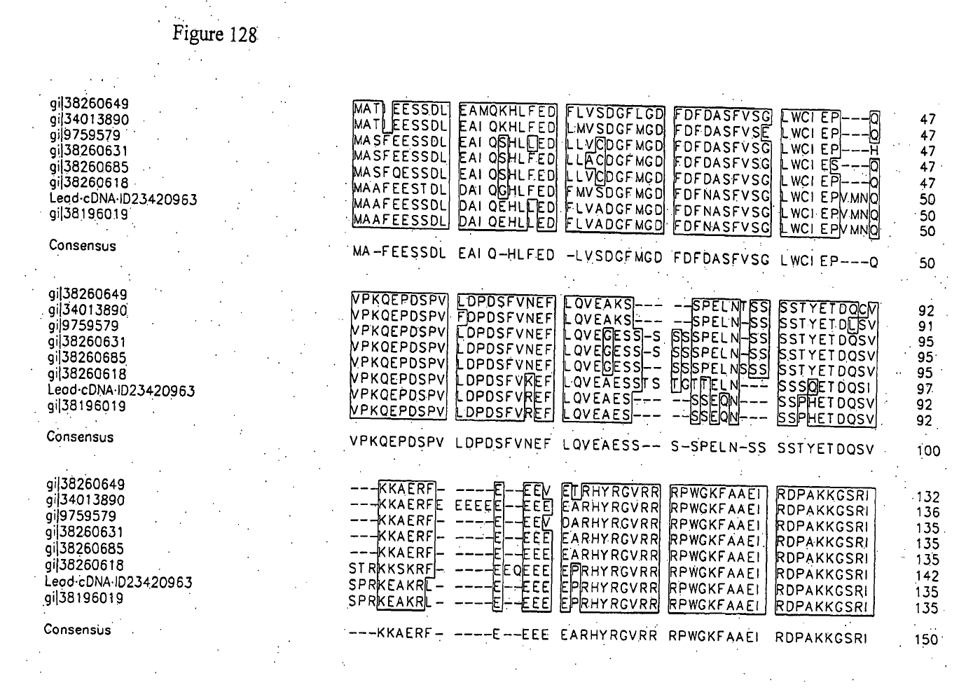

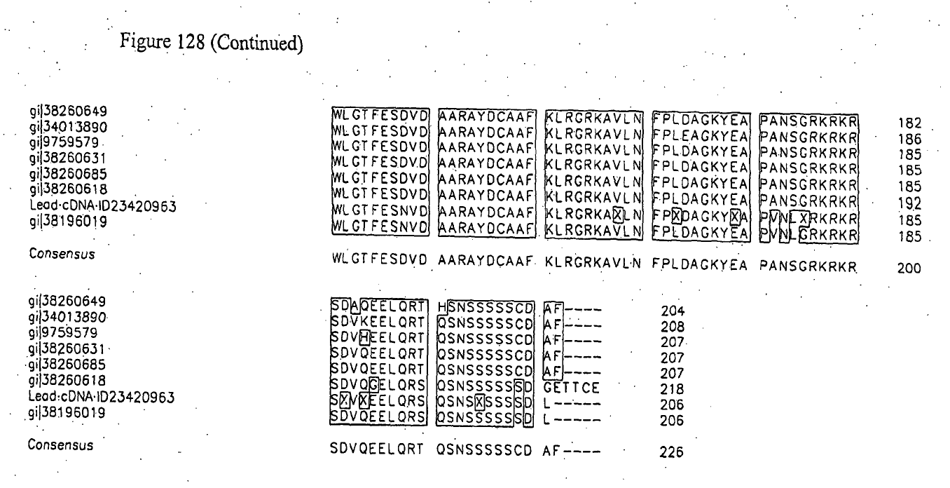

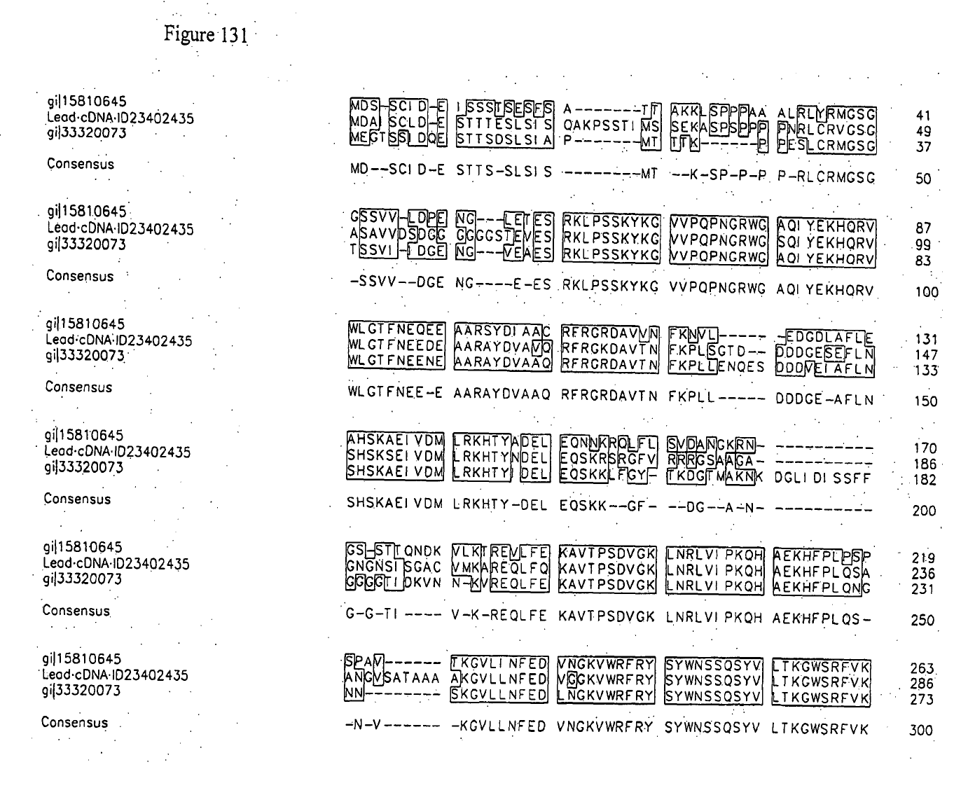

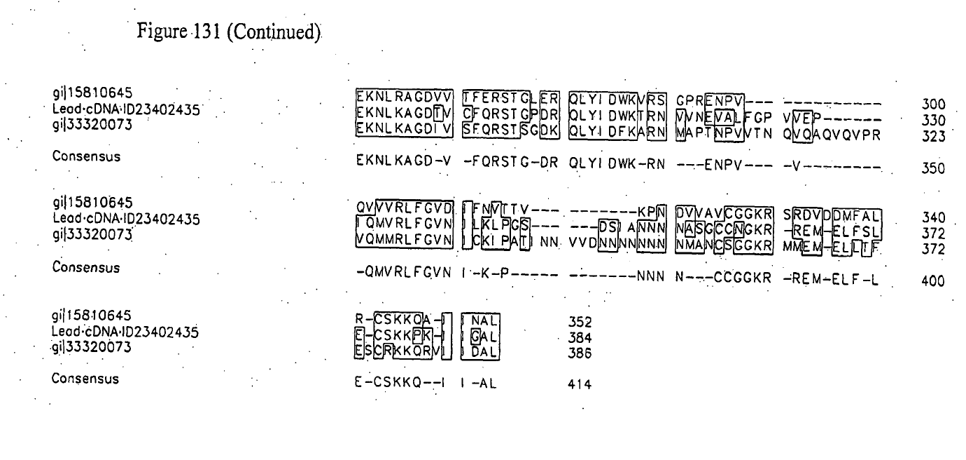

Amino acid sequences of homologs and/or orthologs of the polypeptide having the amino acid sequence set forth in SEQ ID NO:80, SEQ ID NO:246, SEQ ID NO:264, SEQ ID NO:350, SEQ ID NO:874, SEQ ID NO:992, SEQ ID NO:1068, SEQ ID NO:1323, and SEQ ID NO:1358 are provided in FIG. 1, FIG. 19, FIG. 21, FIG. 29, FIG. 89, FIG. 98, FIG. 104, FIG. 128, and FIG. 131, respectively. Each of FIG. 1, FIG. 19, FIG. 21, FIG. 29, FIG. 89, FIG. 98, FIG. 104, FIG. 128, and FIG. 131 also includes a consensus amino acid sequence determined by aligning homologous and/or orthologous amino acid sequences with the amino acid sequence set forth in SEQ ID NO:80, SEQ ID NO:246, SEQ ID NO:264, SEQ ID NO:350, SEQ ID NO:874, SEQ ID NO:992, SEQ ID NO:1068, SEQ ID NO:1323, or SEQ ID NO:1358, respectively.

For example, the alignment in FIG. 1 provides the amino acid sequences of cDNA ID 23798983 (SEQ ID NO:80), CeresClone:916120 (SEQ ID NO:81), CeresClone:464614 (SEQ ID NO:82), and gi|62320596 (SEQ ID NO:83). Other homologs and/or orthologs of SEQ ID NO:80 include Public GI no. 42566740 (SEQ ID NO:84).

The alignment in FIG. 19 provides the amino acid sequences of cDNA ID 23411827 (SEQ ID NO:246), gi|20259679 (SEQ ID NO:247), gi|34900512 (SEQ ID NO:249), gi|51100730 (SEQ ID NO:250), gi|46395277 (SEQ ID NO:251), CeresClone:374770 (SEQ ID NO:252), gi|5081557 (SEQ ID NO:253), gi|53830033 (SEQ ID NO:254), gi|53801434 (SEQ ID NO:255), gi|53830021 (SEQ ID NO:256), gi|53830029 (SEQ ID NO:257), and gi|53830035 (SEQ ID NO:258). Other homologs and/or orthologs of SEQ ID NO:246 include Public GI no. 25354653 (SEQ ID NO:248).

The alignment in FIG. 21 provides the amino acid sequences of cDNA ID 23367111 (SEQ ID NO:264), gi|55585713 (SEQ ID NO:265), gi|30526297 (SEQ ID NO:266), gi|57012875 (SEQ ID NO:267), gi|57012757 (SEQ ID NO:268), CeresClone:953351 (SEQ ID NO:269), gi|4099914 (SEQ ID NO:270), gi|50931913 (SEQ ID NO:271), gi|4099921 (SEQ ID NO:272), gi|37625035 (SEQ ID NO:273), CeresClone:326267 (SEQ ID NO:274), gi|28274832 (SEQ ID NO:275), gi|55824383 (SEQ ID NO:276), CeresClone:554848 (SEQ ID NO:277), gi|55419650 (SEQ ID NO:278), and CeresClone:280241 (SEQ ID NO:279).

The alignment in FIG. 29 provides the amino acid sequences of cDNA ID 23419606 (SEQ ID NO:350) and CeresClone:2347 (SEQ ID NO:352). Other homologs and/or orthologs of SEQ ID NO:350 include Ceres CLONE ID no. 965028 (SEQ ID NO:351), Public GI no. 21592411 (SEQ ID NO:353), and Public GI no. 21387011 (SEQ ID NO:354).

The alignment in FIG. 89 provides the amino acid sequences of cDNA ID 23397999 (SEQ ID NO:874), CeresClone:374770 (SEQ ID NO:875), gi|21717332 (SEQ ID NO:876), gi|11181612 (SEQ ID NO:877), gi|28894445 (SEQ ID NO:878), gi|20259679 (SEQ ID NO:879), gi|42570959 (SEQ ID NO:880), gi|25354653 (SEQ ID NO:881), gi|34900512 (SEQ ID NO:882), gi|13173164 (SEQ ID NO:883), gi|51100730 (SEQ ID NO:884), gi|5081557 (SEQ ID NO:885), gi|53801434 (SEQ ID NO:886), and gi|53830031 (SEQ ID NO:887).

The alignment in FIG. 98 provides the amino acid sequences of cDNA ID 23416775 (SEQ ID NO:992), CeresClone:1091297 (SEQ ID NO:993), gi|33324520 (SEQ ID NO:994), gi|55741382 (SEQ ID NO:995), CeresClone:471446 (SEQ ID NO:996), CeresClone:472054 (SEQ ID NO:997), CeresClone:1050656 (SEQ ID NO:998), and gi|31324058 (SEQ ID NO:999).

The alignment in FIG. 104 provides the amino acid sequences of cDNA ID 23471864 (SEQ ID NO:1068), CeresClone:647941 (SEQ ID NO:1069), CeresClone:1246527 (SEQ ID NO:1070), CeresClone:1306476 (SEQ ID NO:1071), and CeresClone:1259850 (SEQ ID NO:1072).

The alignment in FIG. 128 provides the amino acid sequences of cDNA ID 23420963 (SEQ ID NO:1323), gi|38196019 (SEQ ID NO:1324), gi|38260618 (SEQ ID NO:1325), gi|38260631 (SEQ ID NO:1326), gi|9759579 (SEQ ID NO:1327), gi|38260685 (SEQ ID NO:1328), gi|34013890 (SEQ ID NO:1330), and gi|38260649 (SEQ ID NO:1331). Other homologs and/or orthologs of SEQ ID NO:1323 include Public GI no. 38260669 (SEQ ID NO:1329), Public GI no. 19310643 (SEQ ID NO:1332), and Public GI no. 21554069 (SEQ ID NO:1333).

The alignment in FIG. 131 provides the amino acid sequences of cDNA ID 23402435 (SEQ ID NO:1358), gi|33320073 (SEQ ID NO:1359) and gi|15810645 (SEQ ID NO:1360). Other homologs and/or orthologs of SEQ ID NO:1358 include Ceres CLONE ID no. 38311 (SEQ ID NO:1361), Ceres CLONE ID no. 25854 (SEQ ID NO:1362), Public GI no. 21689705 (SEQ ID NO:1363), Ceres CLONE ID no. 19561 (SEQ ID NO:1364), Public GI no. 21554039 (SEQ ID NO:1365), Public GI no. 20259029 (SEQ ID NO:1366), and Ceres CLONE ID no. 1335983 (SEQ ID NO:1367).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:81-84, SEQ ID NOs:247-258, SEQ ID NOs:265-279, SEQ ID NOs:351-354, SEQ ID NOs:875-887, SEQ ID NOs:993-999, SEQ ID NOs:1069-1072, SEQ ID NOs:1324-1333, SEQ ID NOs:1359-1367, or the consensus sequence set forth in FIG. 1, FIG. 19, FIG. 21, FIG. 29, FIG. 89, FIG. 98, FIG. 104, FIG. 128, or FIG. 131.

A regulatory protein can contain a myb-like DNA binding domain characteristic of myb-like transcription factor polypeptides. The retroviral oncogene v-myb and its cellular counterpart c-myb encode nuclear DNA binding proteins. These proteins belong to the SANT domain family that specifically recognize the sequence YAAC(G/T)G. In myb, one of the most conserved regions consisting of three tandem repeats has been shown to be involved in DNA binding. SEQ ID NO:721, SEQ ID NO:769, SEQ ID NO:797, SEQ ID NO:820, SEQ ID NO:1074, SEQ ID NO:1087, SEQ ID NO:1261, and SEQ ID NO:1353 set forth the amino acid sequences of DNA clones, identified herein as cDNA ID 23417641 (SEQ ID NO:720), cDNA ID 23792467 (SEQ ID NO:768), cDNA ID 23765347 (SEQ ID NO:796), cDNA ID 23751503 (SEQ ID NO:819), cDNA ID 23370870 (SEQ ID NO:1073), cDNA ID 23361688 (SEQ ID NO:1086), cDNA ID 23449314 (SEQ ID NO:1260), and cDNA ID 23377150 (SEQ ID NO:1352), respectively, that are predicted to encode myb-like transcription factor polypeptides.

In some cases, a regulatory containing a myb-like DNA binding domain and a Linker_histone domain characteristic of polypeptides belonging to the linker histone H1 and H5 family. Linker histone H1 is an essential component of chromatin structure. H1 links nucleosomes into higher order structures. Histone H5 performs the same function as histone H1 and replaces H1 in certain cells. The structure of GH5, the globular domain of the linker histone H5, is known. The fold is similar to the DNA-binding domain of the catabolite gene activator protein, CAP, thus providing a possible model for the binding of GH5 to DNA. SEQ ID NO:288 sets forth the amino acid sequence of a DNA clone, identified herein as cDNA ID 23376150 (SEQ ID NO:287), that is predicted to encode a polypeptide containing a myb-like DNA binding domain and a Linker_histone domain.

A regulatory protein can comprise the amino acid sequence set forth in SEQ ID NO:721, SEQ ID NO:769, SEQ ID NO:797, SEQ ID NO:820, SEQ ID NO:1074, SEQ ID NO:1087, SEQ ID NO:1261, SEQ ID NO:1353, or SEQ ID NO:288. Alternatively, a regulatory protein can be a homolog, ortholog, or variant of the polypeptide having the amino acid sequence set forth in SEQ ID NO:721, SEQ ID NO:769, SEQ ID NO:797, SEQ ID NO:820, SEQ ID NO:1074, SEQ ID NO:1087, SEQ ID NO:1261, SEQ ID NO:1353, or SEQ ID NO:288. For example, a regulatory protein can have an amino acid sequence with at least 40% sequence identity, e.g., 40%, 41%, 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:721, SEQ ID NO:769, SEQ ID NO:797, SEQ ID NO:820, SEQ ID NO:1074, SEQ ID NO:1087, SEQ ID NO:1261, SEQ ID NO:1353, or SEQ ID NO:288.

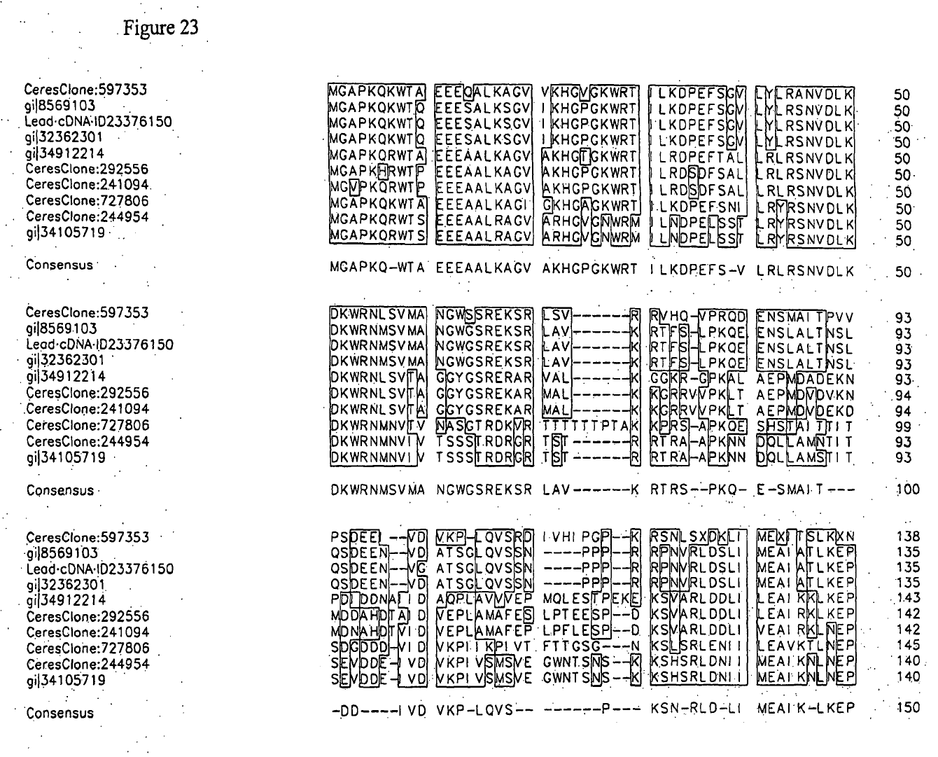

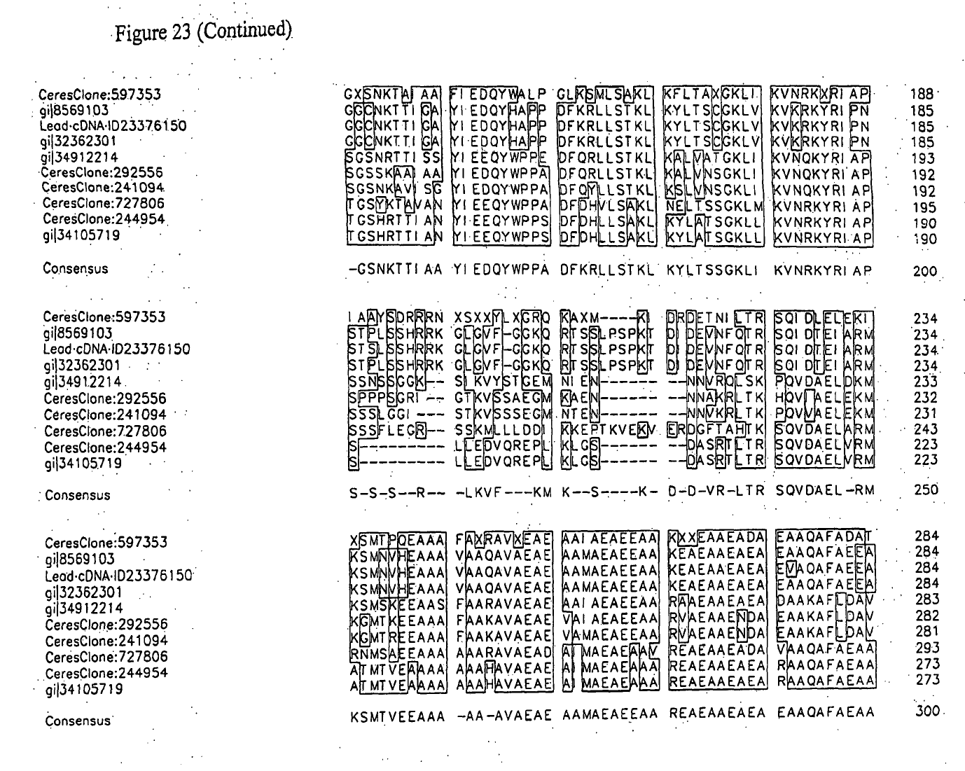

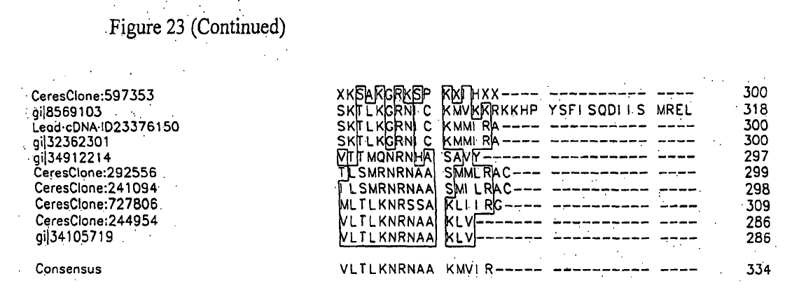

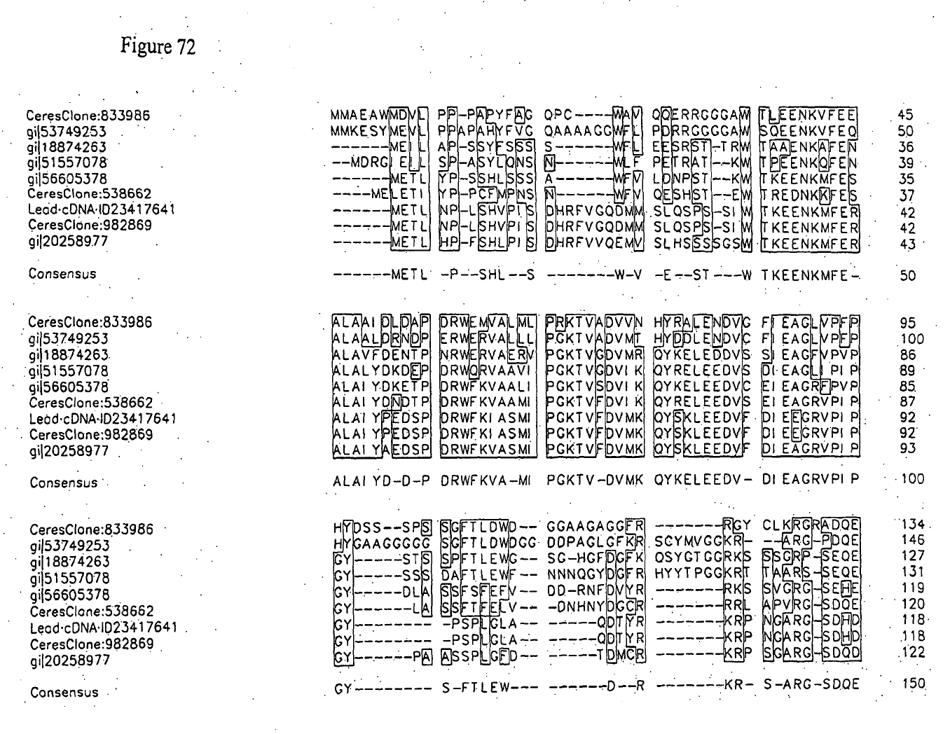

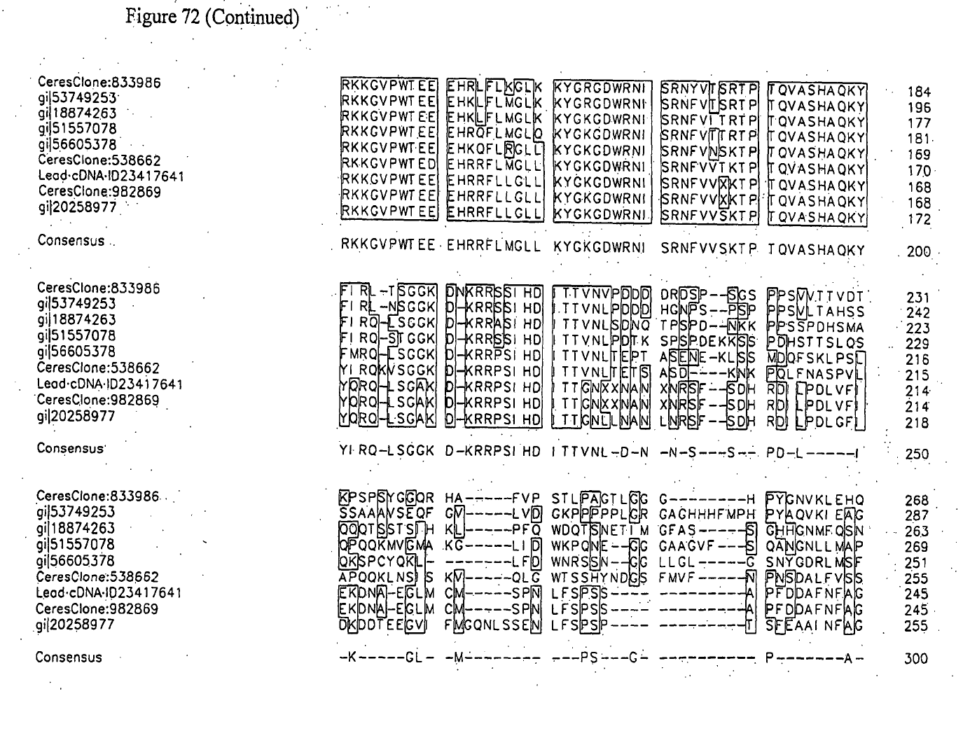

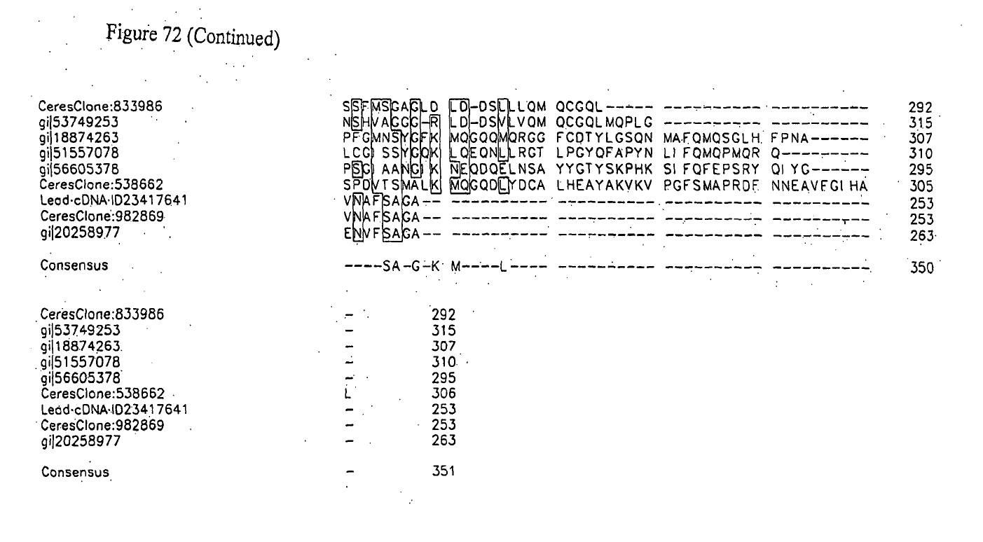

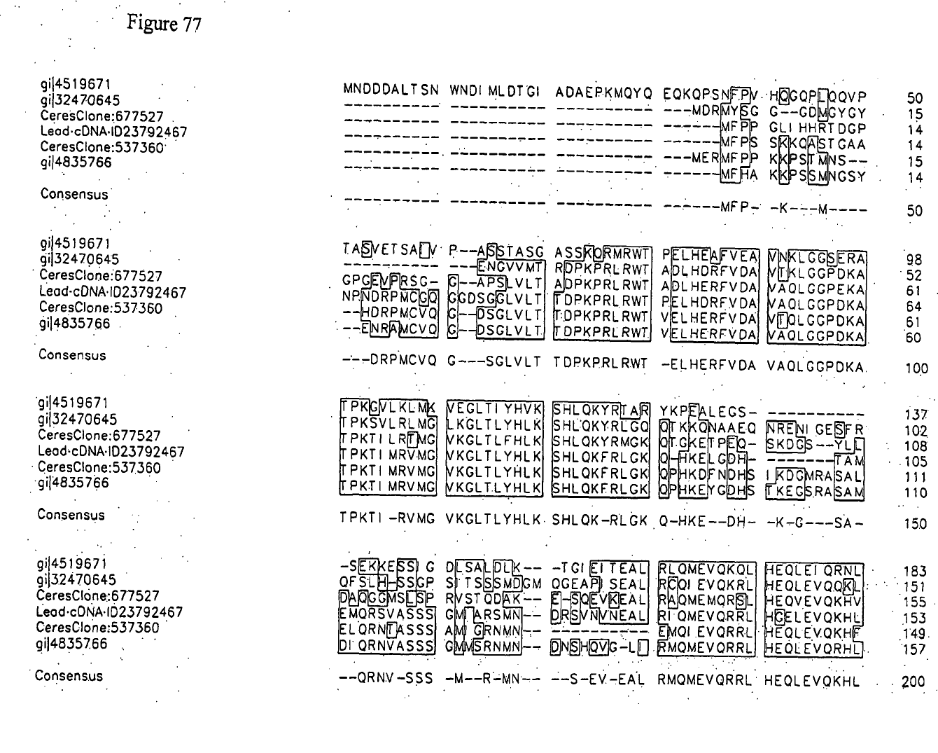

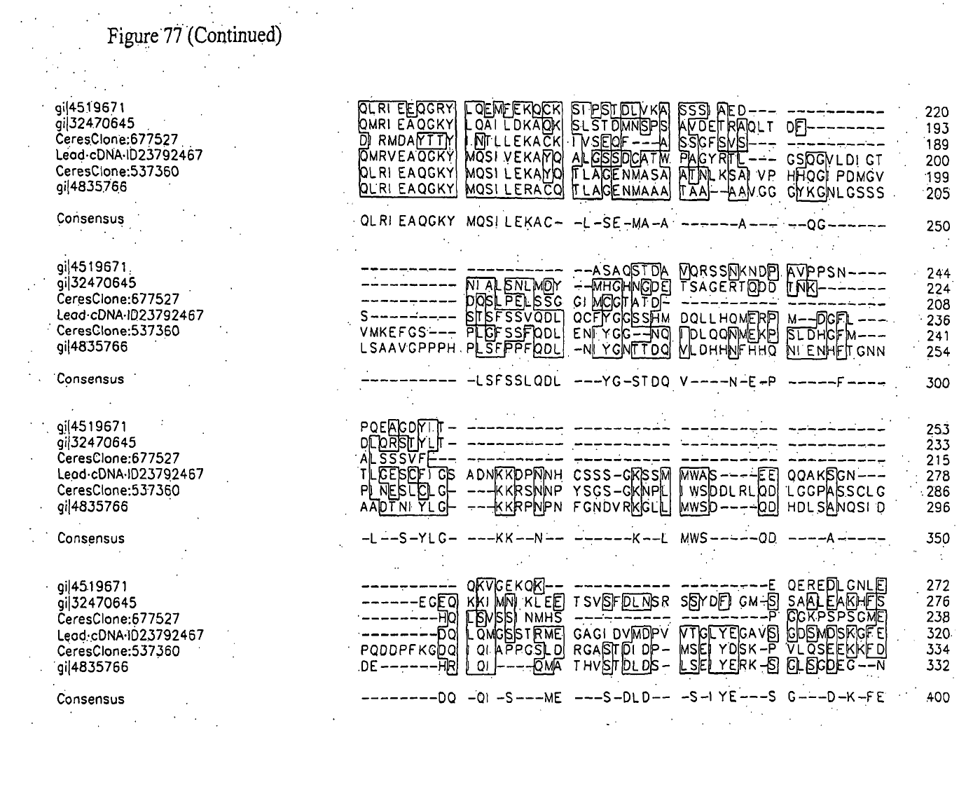

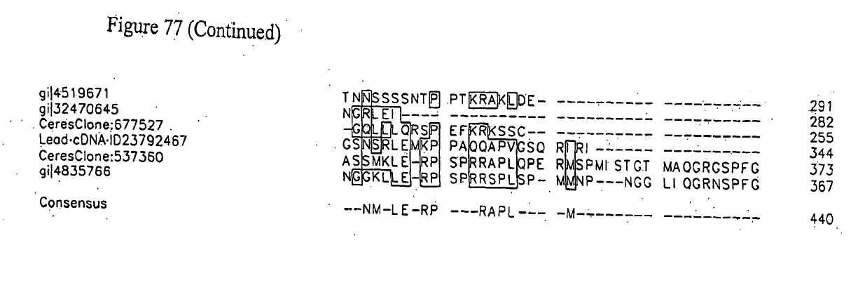

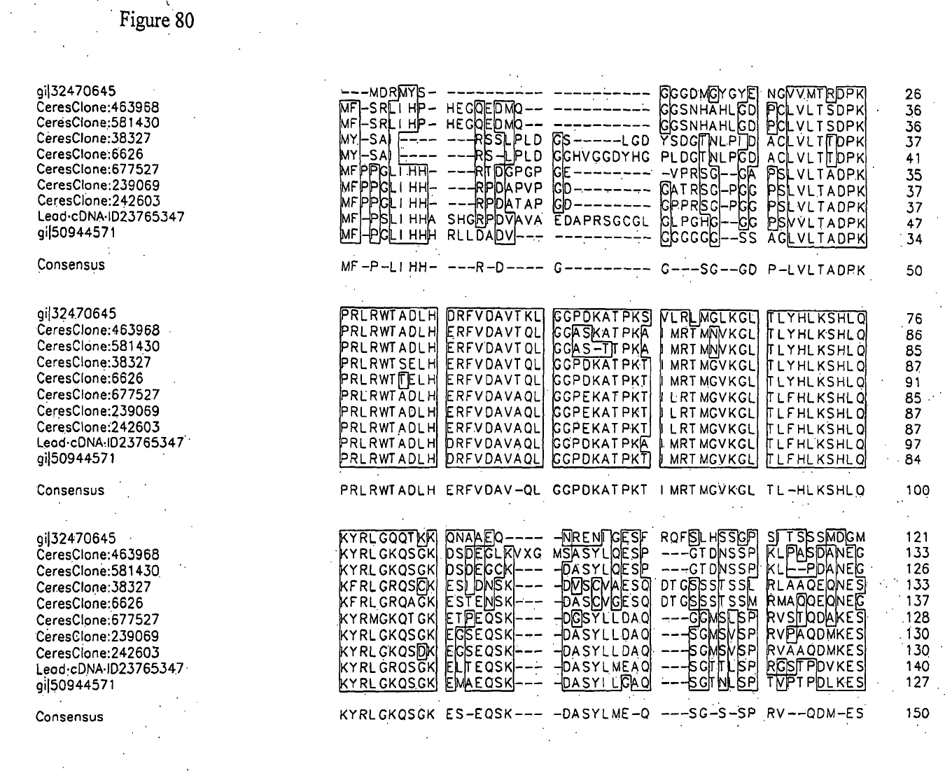

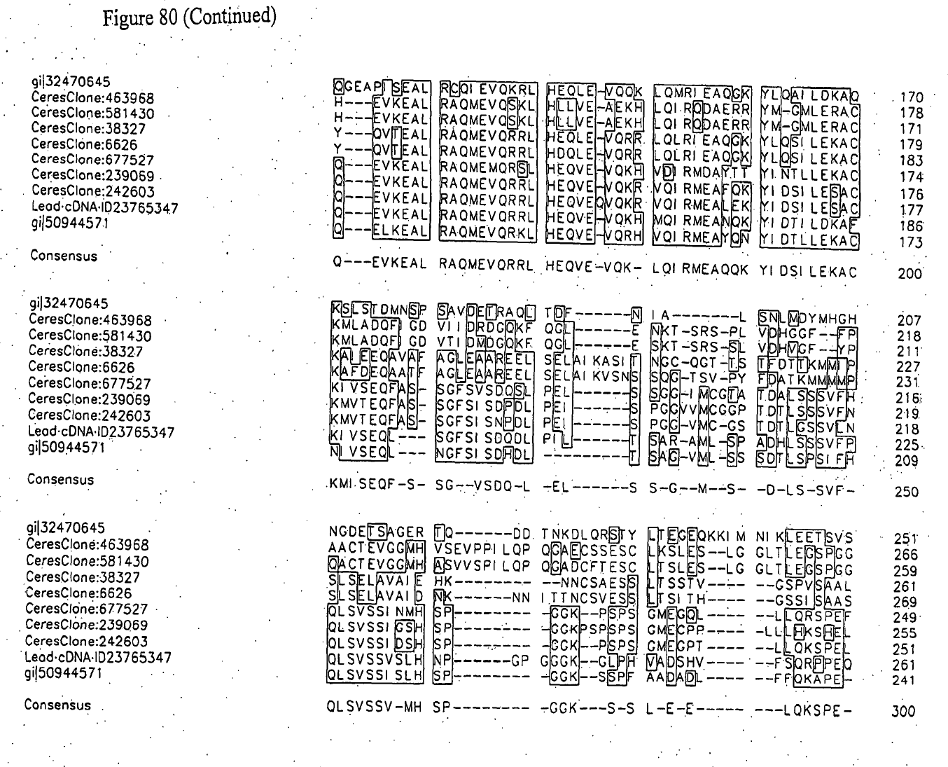

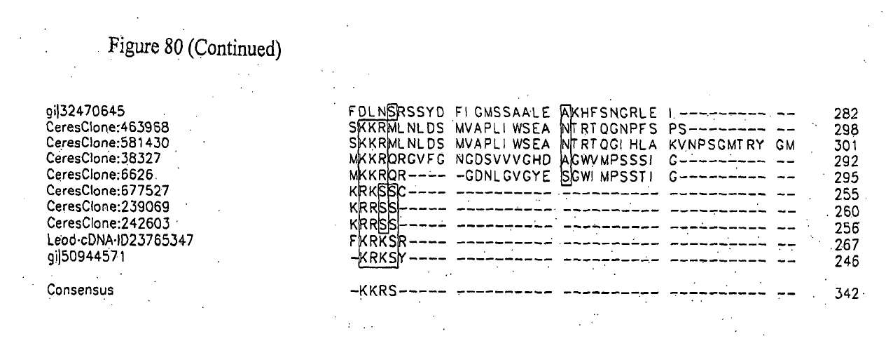

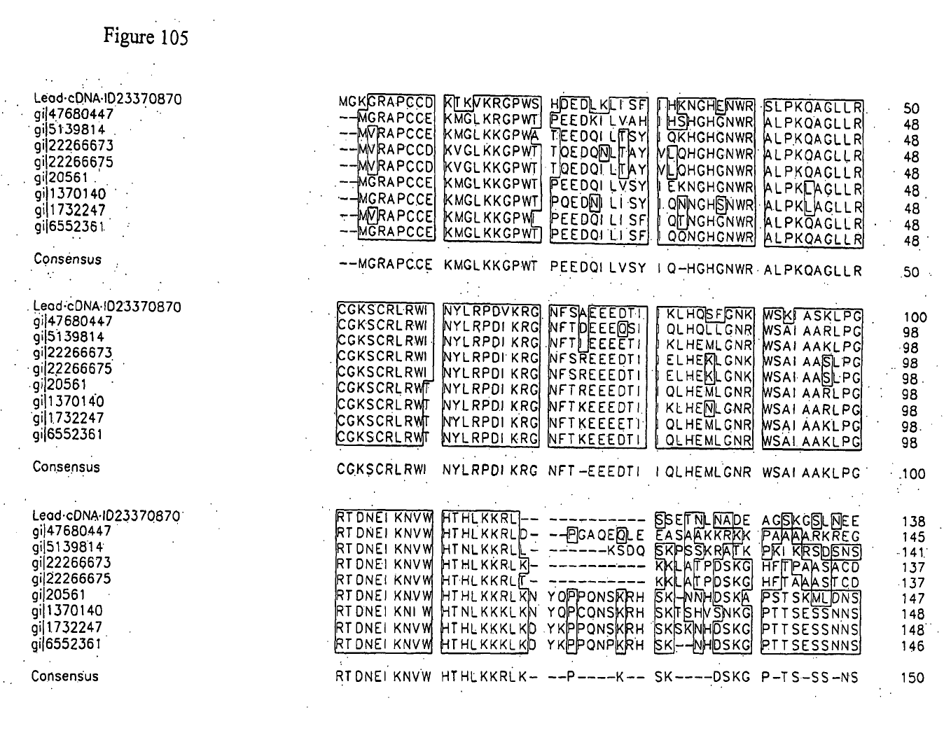

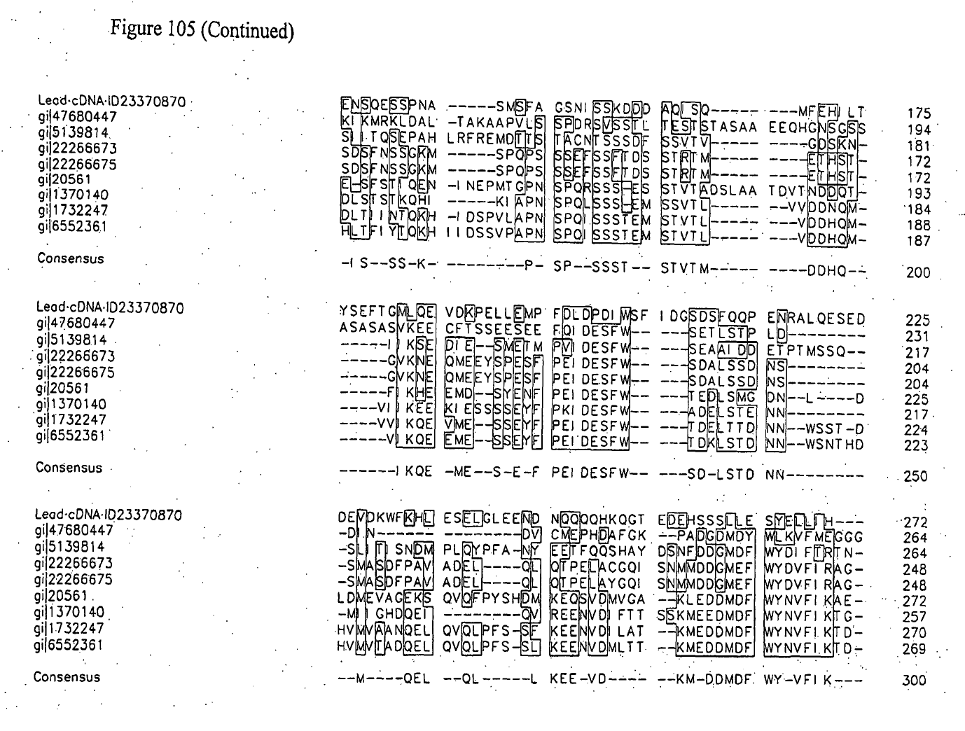

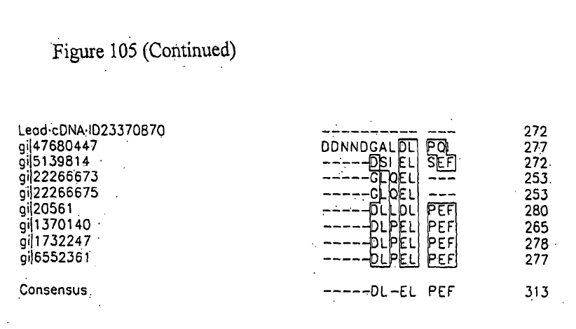

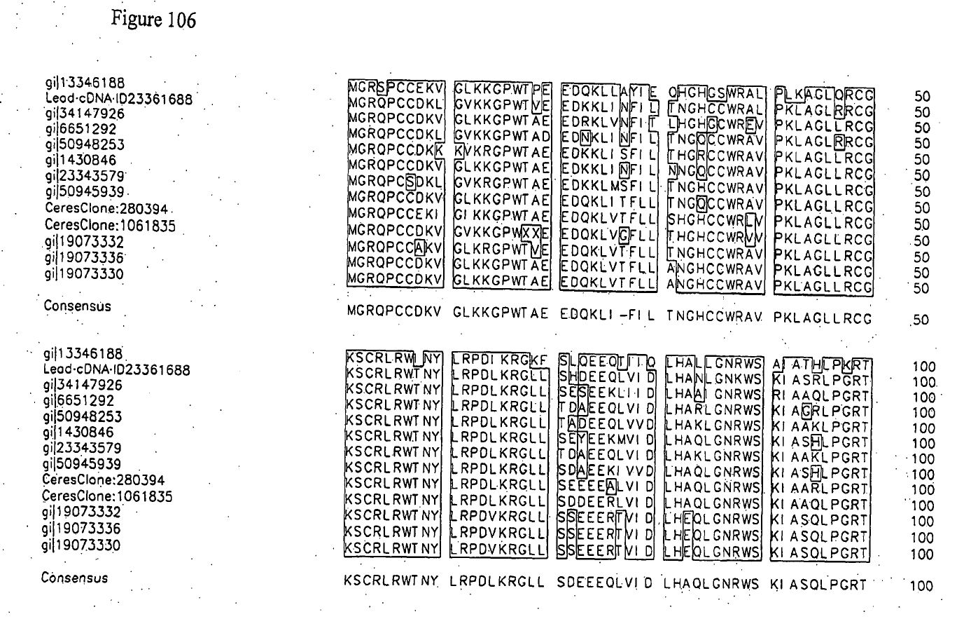

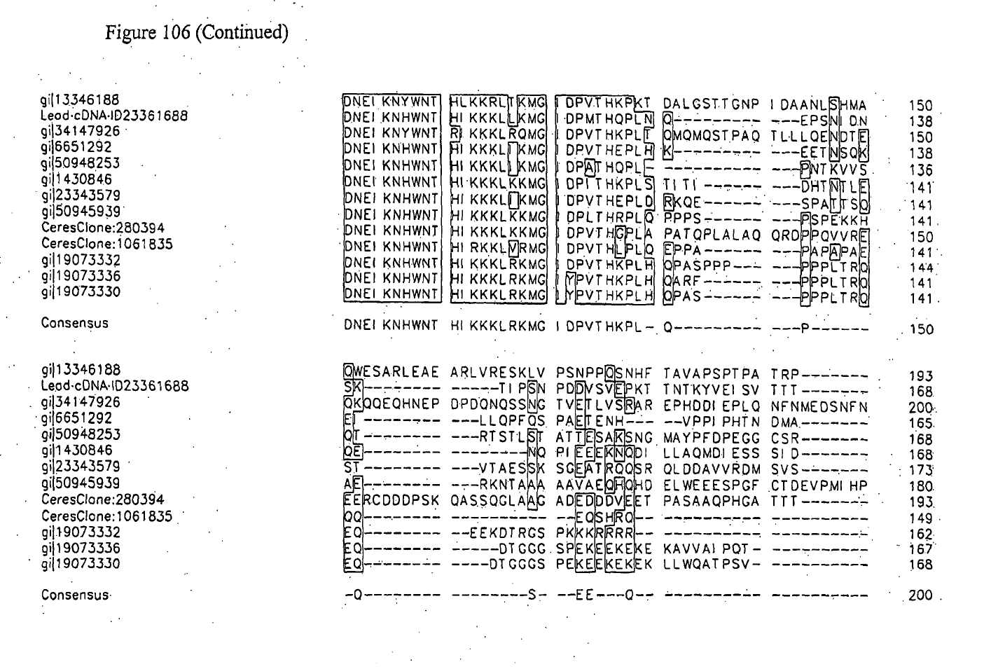

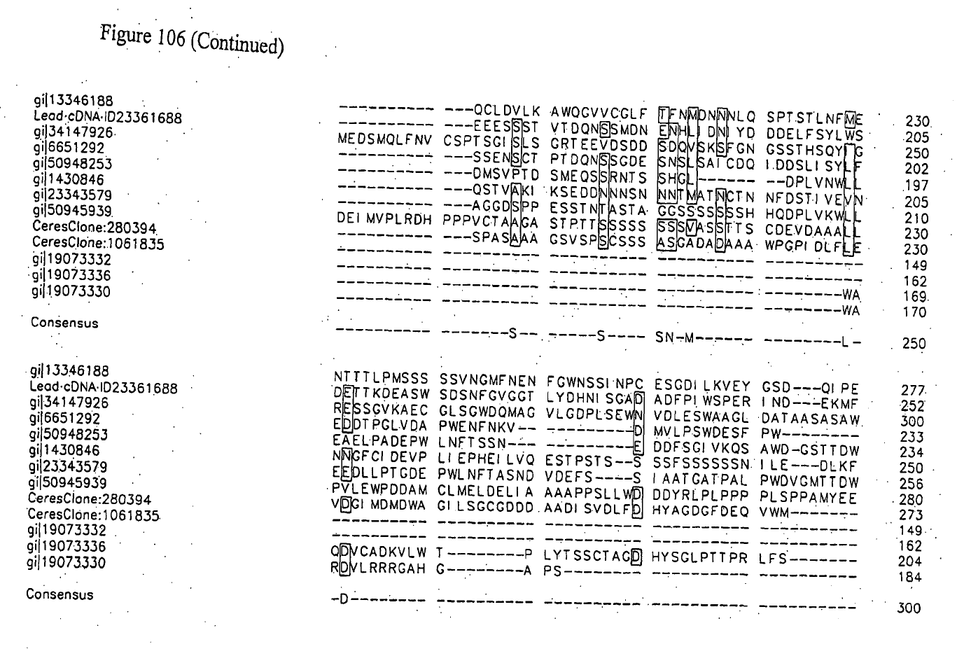

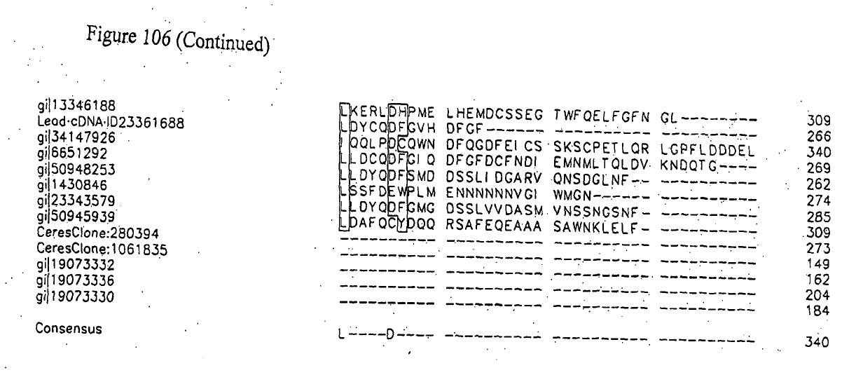

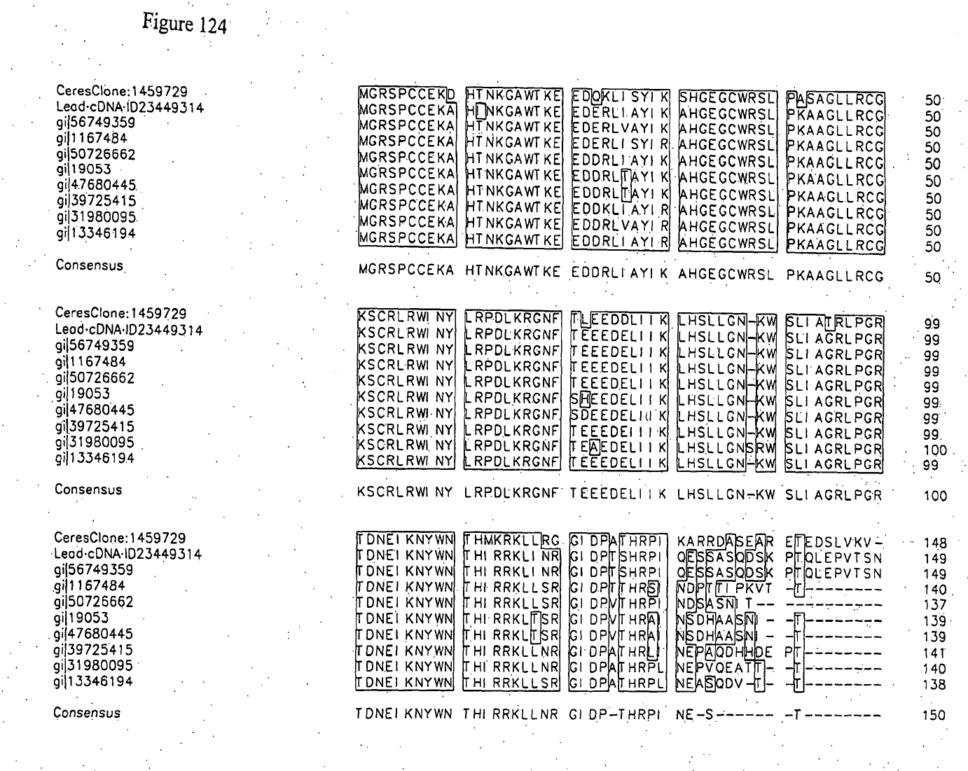

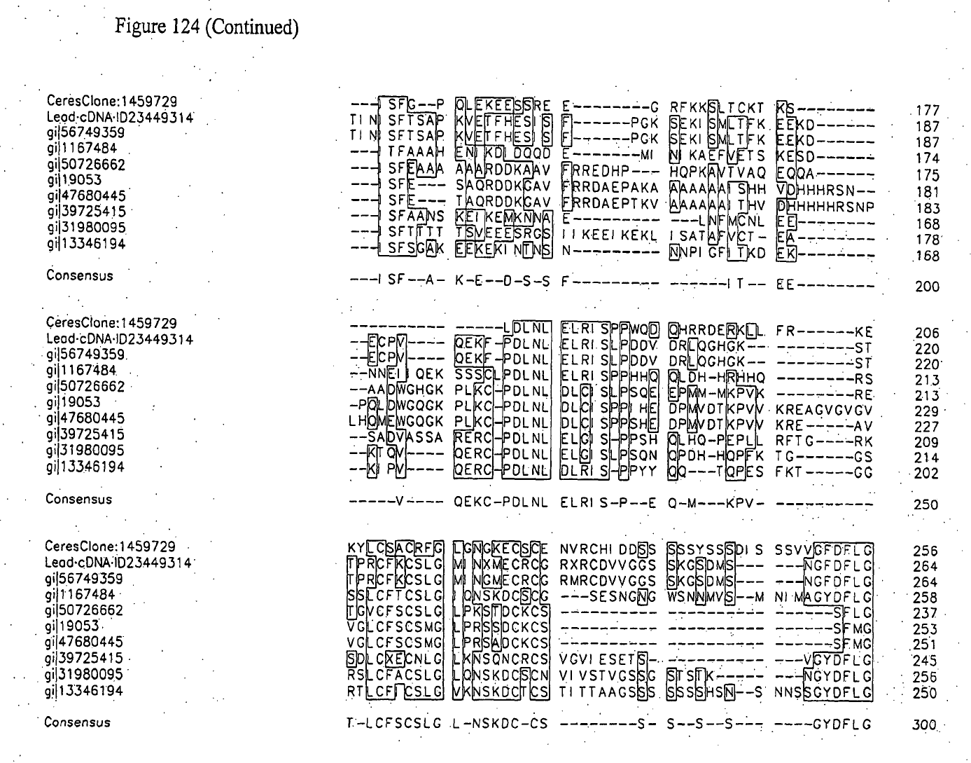

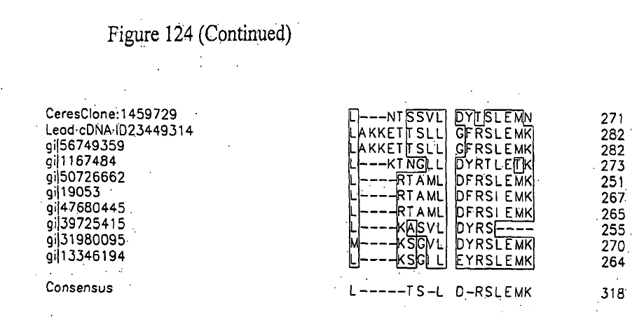

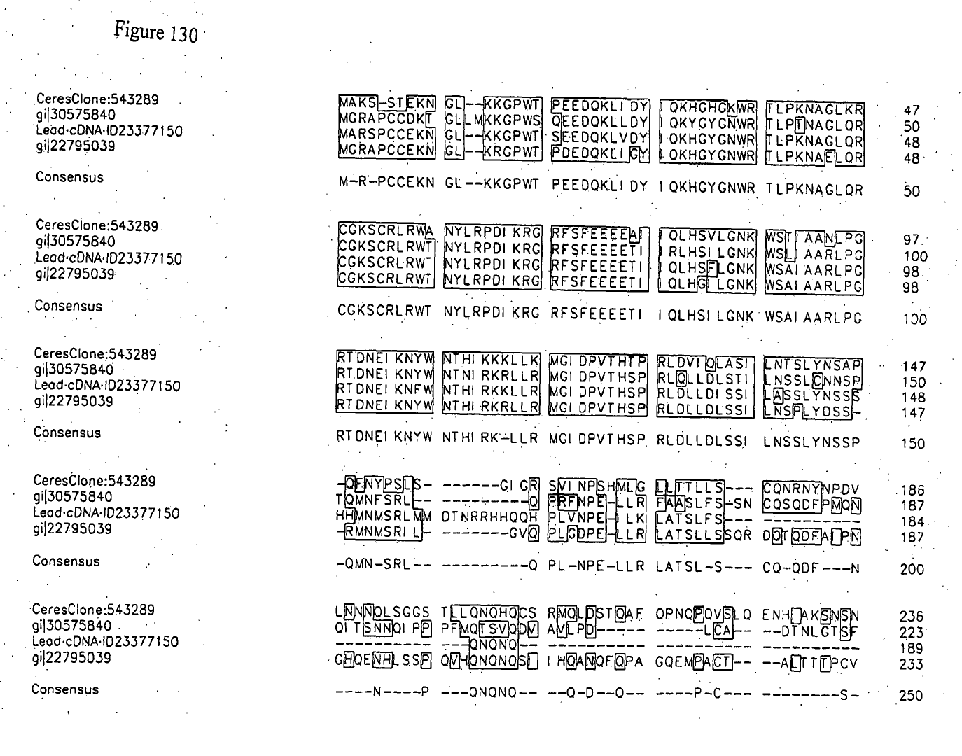

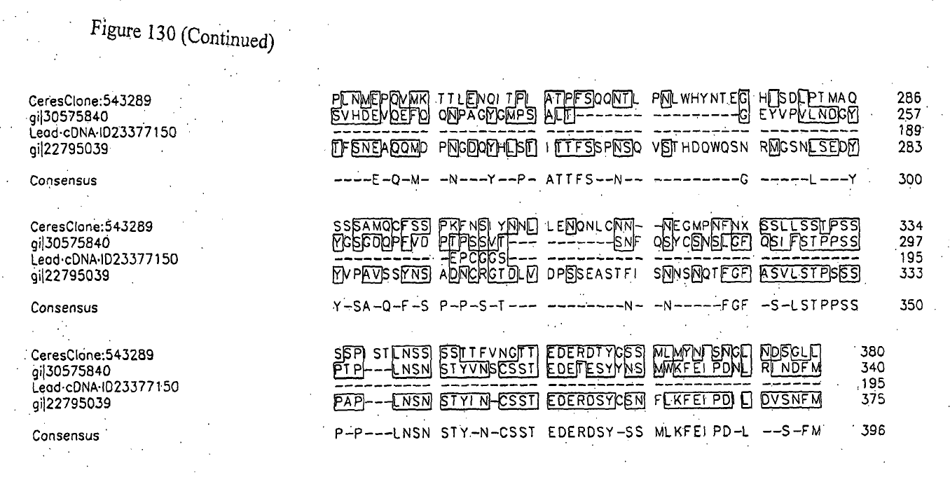

Amino acid sequences of homologs and/or orthologs of the polypeptide having the amino acid sequence set forth in SEQ ID NO:721, SEQ ID NO:769, SEQ ID NO:797, SEQ ID NO:1074, SEQ ID NO:1087, SEQ ID NO:1261, SEQ ID NO:1353, and SEQ ID NO:288 are provided in FIG. 72, FIG. 77, FIG. 80, FIG. 105, FIG. 106, FIG. 124, FIG. 130, and FIG. 23, respectively. Each of FIG. 72, FIG. 77, FIG. 80, FIG. 105, FIG. 106, FIG. 124, FIG. 130, and FIG. 23 also includes a consensus amino acid sequence determined by aligning homologous and/or orthologous amino acid sequences with the amino acid sequence set forth in SEQ ID NO:721, SEQ ID NO:769, SEQ ID NO:797, SEQ ID NO:1074, SEQ ID NO:1087, SEQ ID NO:1261, SEQ ID NO:1353, or SEQ ID NO:288, respectively.

For example, the alignment in FIG. 72 provides the amino acid sequences of cDNA ID 23417641 (SEQ ID NO:721), CeresClone:982869 (SEQ ID NO:722), gi|20258977 (SEQ ID NO:723), CeresClone:538662 (SEQ ID NO:724), gi|18874263 (SEQ ID NO:725), gi|56605378 (SEQ ID NO:726), gi|51557078 (SEQ ID NO:727), CeresClone:833986 (SEQ ID NO:729), and gi|53749253 (SEQ ID NO:730). Other homologs and/or orthologs of SEQ ID NO:721 include Public GI no. 12005328 (SEQ ID NO:728).

The alignment in FIG. 77 provides the amino acid sequences of cDNA ID 23792467 (SEQ ID NO:769), gi|32470645 (SEQ ID NO:770), CeresClone:537360 (SEQ ID NO:771), gi|4835766 (SEQ ID NO:773), CeresClone:677527 (SEQ ID NO:774), and gi|4519671 (SEQ ID NO:775). Other homologs and/or orthologs of SEQ ID NO:769 include Public GI no. 30699418 (SEQ ID NO:772).

The alignment in FIG. 80 provides the amino acid sequences of cDNA ID 23765347 (SEQ ID NO:797), gi|50944571 (SEQ ID NO:798), CeresClone:239069 (SEQ ID NO:799), CeresClone:677527 (SEQ ID NO:800), CeresClone:242603 (SEQ ID NO:802), CeresClone:38327 (SEQ ID NO:803), CeresClone:463968 (SEQ ID NO:805), CeresClone:6626 (SEQ ID NO:806), CeresClone:581430 (SEQ ID NO:809), and gi|32470645 (SEQ ID NO:810). Other homologs and/or orthologs of SEQ ID NO:797 include Ceres CLONE ID no. 317477 (SEQ ID NO:801), Public GI no. 21593358 (SEQ ID NO:804), Public GI no. 21594046 (SEQ ID NO:807), and Public GI no. 42572521 (SEQ ID NO:808).

The alignment in FIG. 105 provides the amino acid sequences of cDNA ID 23370870 (SEQ ID NO:1074), gi|47680447 (SEQ ID NO:1075), gi|1370140 (SEQ ID NO:1078), gi|20561 (SEQ ID NO:1079), gi|22266673 (SEQ ID NO:1081), gi|22266675 (SEQ ID NO:1082), gi|1732247 (SEQ ID NO:1083), gi|5139814 (SEQ ID NO:1084), and gi|6552361 (SEQ ID NO:1085). Other homologs and/or orthologs of SEQ ID NO:1074 include Ceres CLONE ID no. 540373 (SEQ ID NO:1076), Ceres CLONE ID no. 347485 (SEQ ID NO:1077), and Public GI no. 32489375 (SEQ ID NO:1080).

The alignment in FIG. 106 provides the amino acid sequences of cDNA ID 23361688 (SEQ ID NO:1087), CeresClone:280394 (SEQ ID NO:1088), gi|50945939 (SEQ ID NO:1089), gi|19073336 (SEQ ID NO:1090), gi|19073332 (SEQ ID NO:1091), CeresClone:1061835 (SEQ ID NO:1092), gi|19073330 (SEQ ID NO:1093), gi|13346188 (SEQ ID NO:1094), gi|6651292 (SEQ ID NO:1095), gi|1430846 (SEQ ID NO:1096), gi|34147926 (SEQ ID NO:1097), gi|50948253 (SEQ ID NO:1098), and gi|23343579 (SEQ ID NO:1100). Other homologs and/or orthologs of SEQ ID NO:1087 include Public GI no. 50725788 (SEQ ID NO:1099).

The alignment in FIG. 124 provides the amino acid sequences of cDNA ID 23449314 (SEQ ID NO:1261), gi|56749359 (SEQ ID NO:1262), gi|13346194 (SEQ ID NO:1267), gi|39725415 (SEQ ID NO:1269), gi|31980095 (SEQ ID NO:1270), gi|1167484 (SEQ ID NO:1271), gi|50726662 (SEQ ID NO:1272), gi|19053 (SEQ ID NO:1273), CeresClone:1459729 (SEQ ID NO:1276), and gi|47680445 (SEQ ID NO:1277). Other homologs and/or orthologs of SEQ ID NO:1261 include Public GI no. 3941412 (SEQ ID NO:1263), Public GI no. 28628965 (SEQ ID NO:1264), Ceres CLONE ID no. 1560573 (SEQ ID NO:1265), Public GI no. 82308 (SEQ ID NO:1266), Public GI no. 42541167 (SEQ ID NO:1268), Public GI no. 19072766 (SEQ ID NO:1274), and Public GI no. 50948275 (SEQ ID NO:1275).

The alignment in FIG. 130 provides the amino acid sequences of cDNA ID 23377150 (SEQ ID NO:1353), gi|30575840 (SEQ ID NO:1354), gi|22795039 (SEQ ID NO:1355), and CeresClone:543289 (SEQ ID NO:1356).

The alignment in FIG. 23 provides the amino acid sequences of cDNA ID 23376150 (SEQ ID NO:288), gi|32362301 (SEQ ID NO:289), gi|8569103 (SEQ ID NO:290), CeresClone:597353 (SEQ ID NO:291), CeresClone:244954 (SEQ ID NO:292), gi|34105719 (SEQ ID NO:294), gi|34912214 (SEQ ID NO:295), CeresClone:292556 (SEQ ID NO:296), CeresClone:241094 (SEQ ID NO:298), and CeresClone:727806 (SEQ ID NO:299). Other homologs and/or orthologs include Public GI no. 34105723 (SEQ ID NO:293) and Public GI no. 33286863 (SEQ ID NO:297).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to SEQ ID NOs:722-730, SEQ ID NOs:770-775, SEQ ID NOs:798-810, SEQ ID NOs:1075-1085, SEQ ID NOs:1088-1100, SEQ ID NOs:1262-1277, SEQ ID NOs:1354-1356, SEQ ID NOs:289-299, or the consensus sequence set forth in FIG. 72, FIG. 77, FIG. 80, FIG. 105, FIG. 106, FIG. 124, FIG. 130, or FIG. 23.

A regulatory protein can have one or more domains characteristic of a basic-leucine zipper (bZIP) transcription factor polypeptide. For example, a regulatory protein can have a bZIP_{—}1 domain. The bZIP transcription factor polypeptides of eukaryotes contain a basic region mediating sequence-specific DNA binding and a leucine zipper region that is required for dimerization. In plants, bZIP transcription factors regulate processes including pathogen defense, light and stress signaling, seed maturation and flower development. The Arabidopsis genome sequence contains at least 70 distinct members of the bZIP family. SEQ ID NO:113, SEQ ID NO:144, and SEQ ID NO:565 set forth the amino acid sequences of DNA clones, identified herein as cDNA ID 23698626 (SEQ ID NO:112), cDNA ID 23499985 (SEQ ID NO:143), and cDNA ID 23660778 (SEQ ID NO:564) respectively, each of which is predicted to encode a polypeptide containing a bZIP_{—}1 domain.

In some cases, a regulatory protein can contain a bZIP_{—}2 domain characteristic of a bZIP transcription factor polypeptide. SEQ ID NO:152 and SEQ ID NO:523 set forth the amino acid sequences of DNA clones, identified herein as cDNA ID 23651179 and cDNA ID 23357846, respectively, each of which is predicted to encode a polypeptide containing a bZIP_{—}2 domain.

In some cases, a regulatory protein can contain a bZIP_{—}1 domain and a bZIP_{—}2 domain. SEQ ID NO:1026 sets forth the amino acid sequence of a DNA clone, identified herein as cDNA ID 23359443 (SEQ ID NO:1025), that is predicted to encode a polypeptide containing a bZIP_{—}1 domain and a bZIP_{—}2 domain.

A regulatory protein can comprise the amino acid sequence set forth in SEQ ID NO:113, SEQ ID NO:144, SEQ ID NO:565, SEQ ID NO:152, SEQ ID NO:523, or SEQ ID NO:1026. Alternatively, a regulatory protein can be a homolog, ortholog, or variant of the polypeptide having the amino acid sequence set forth in SEQ ID NO:113, SEQ ID NO:144, SEQ ID NO:565, SEQ ID NO:152, SEQ ID NO:523, or SEQ ID NO:1026. For example, a regulatory protein can have an amino acid sequence with at least 35% sequence identity, e.g., 36%, 39%, 41%, 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:113, SEQ ID NO:144, SEQ ID NO:565, SEQ ID NO:152, SEQ ID NO:523, or SEQ ID NO:1026.

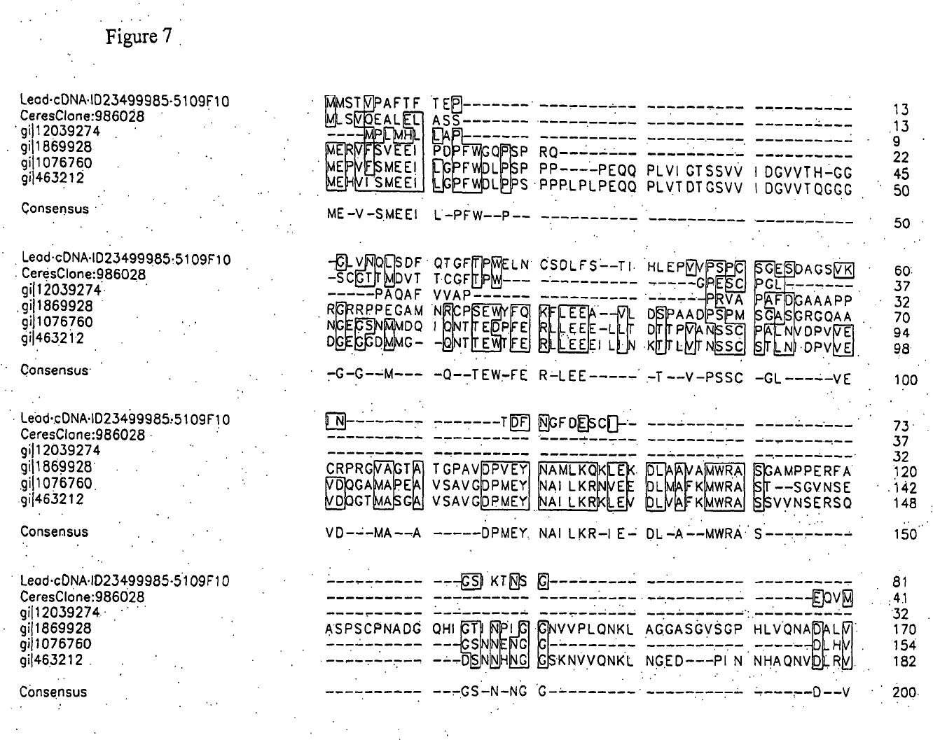

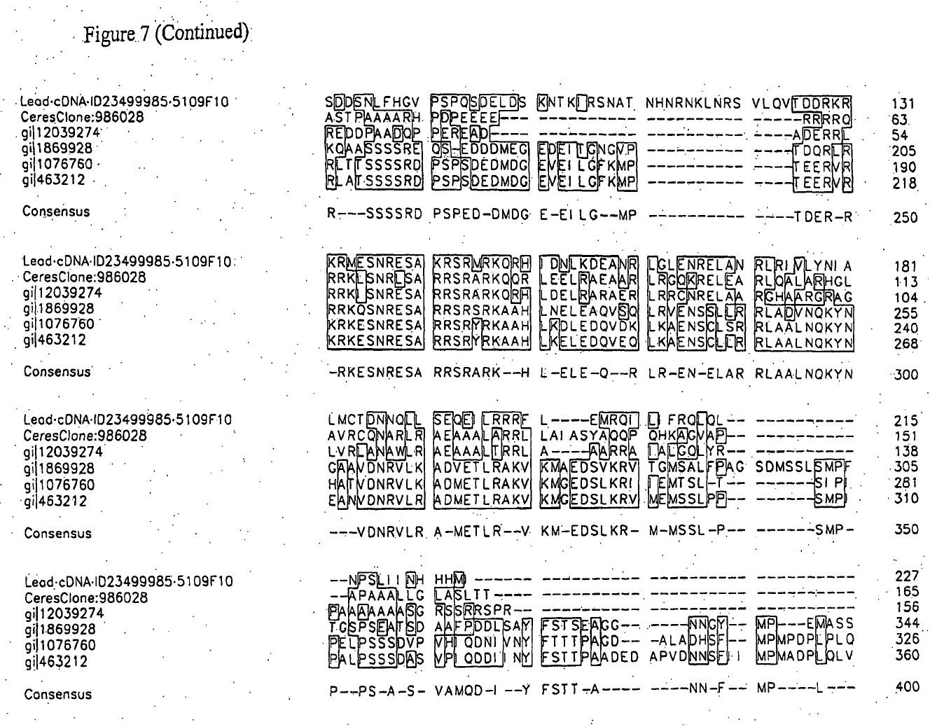

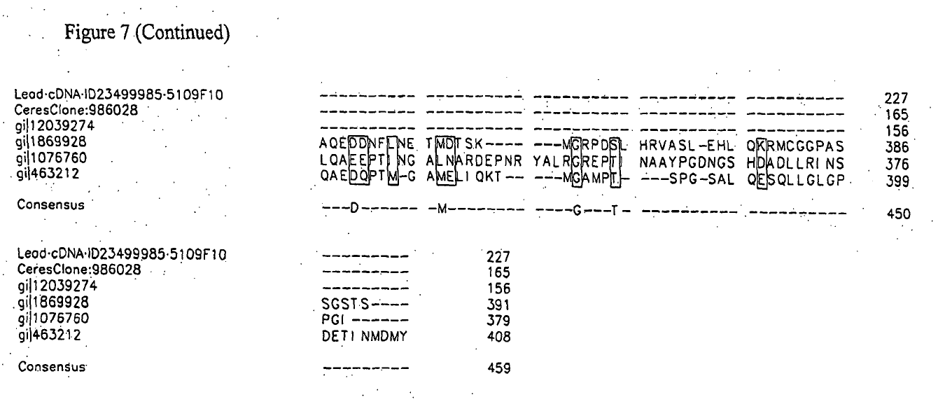

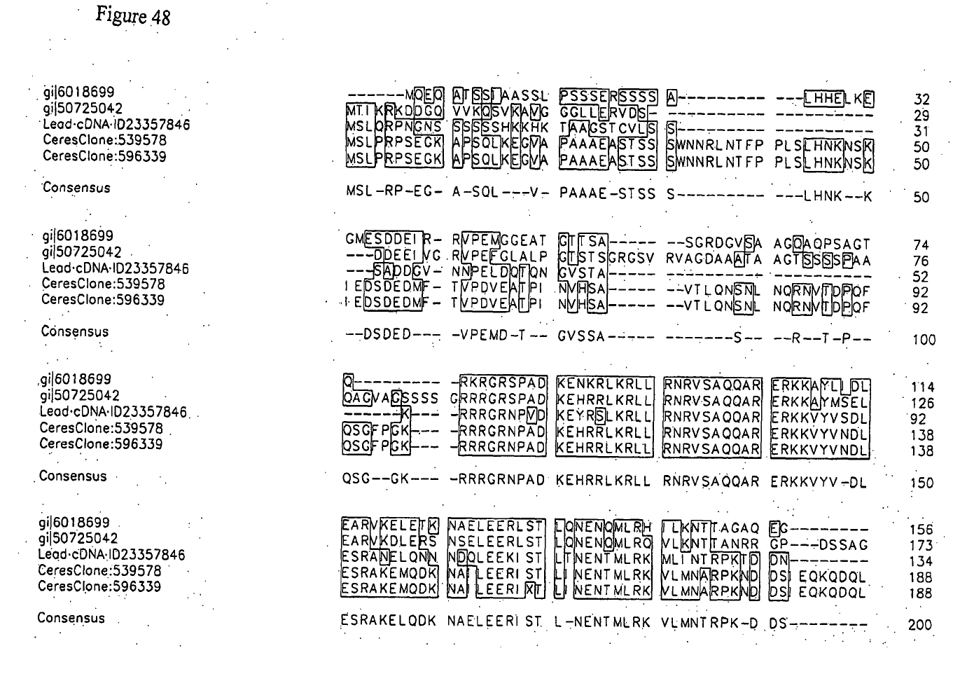

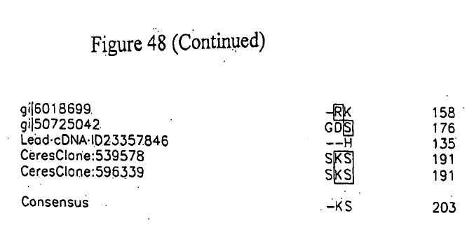

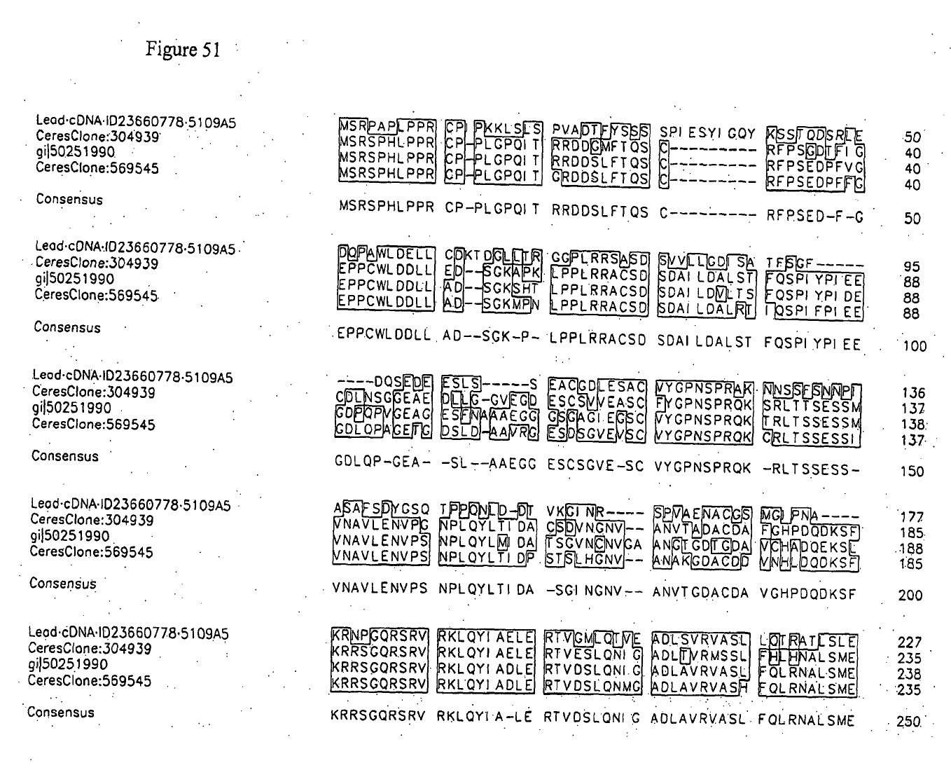

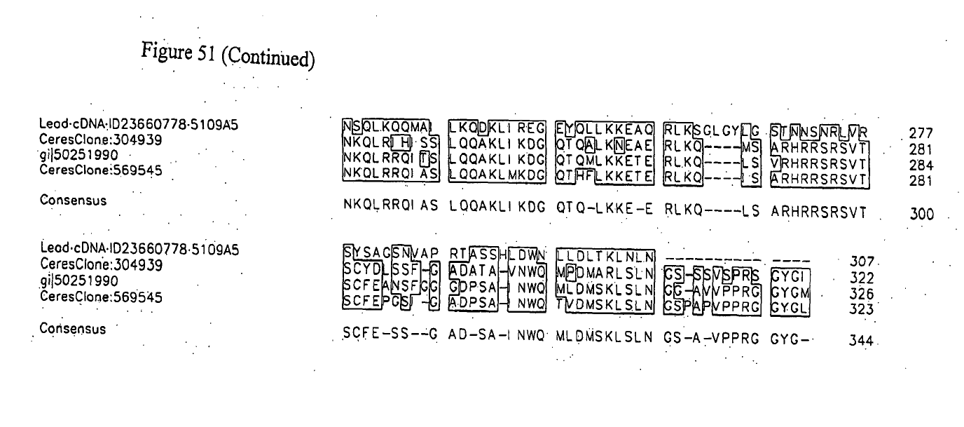

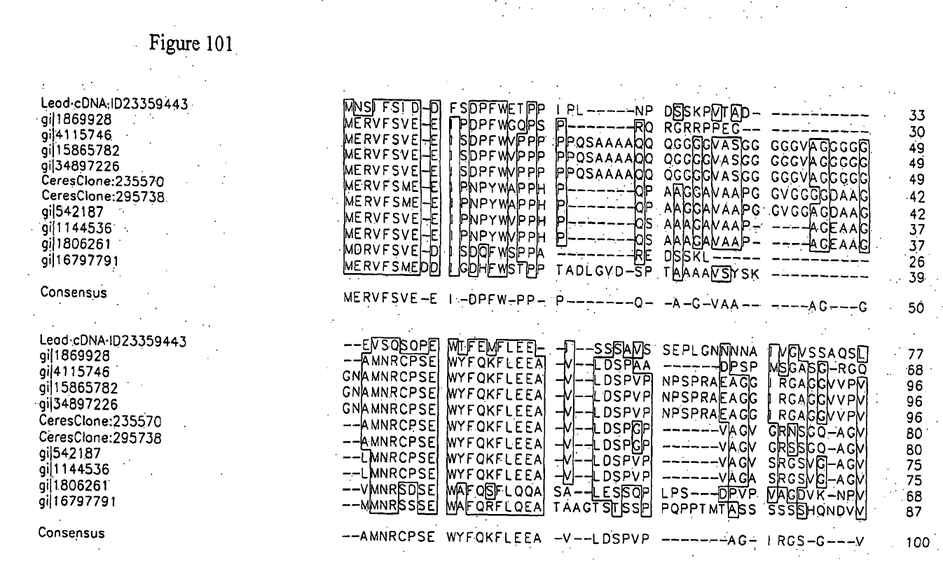

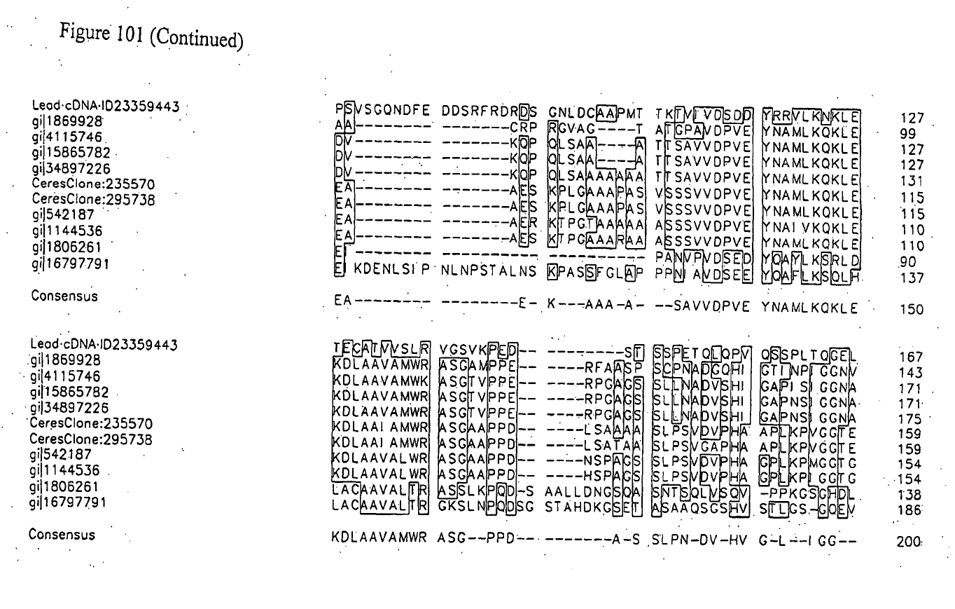

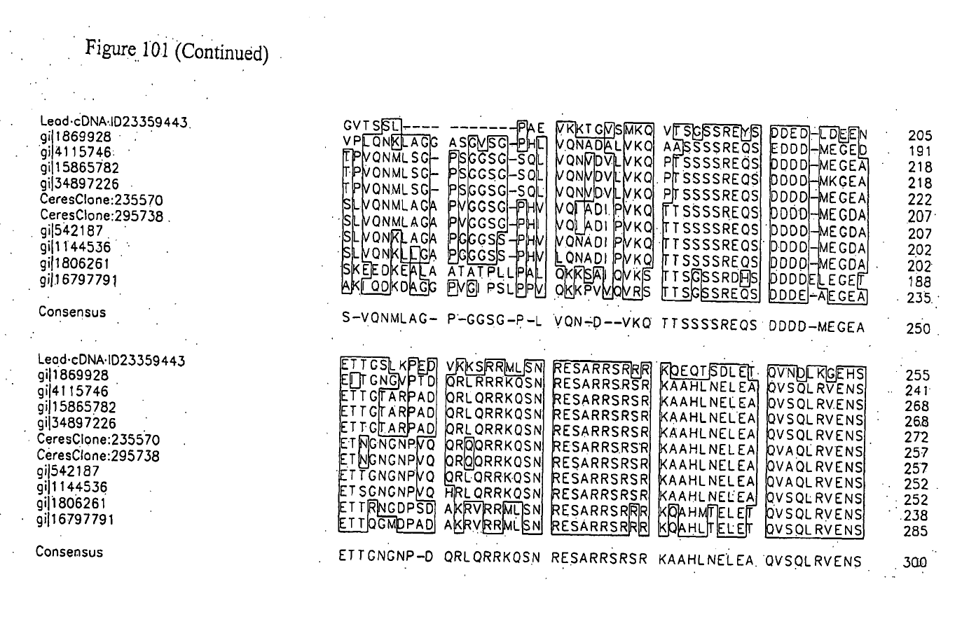

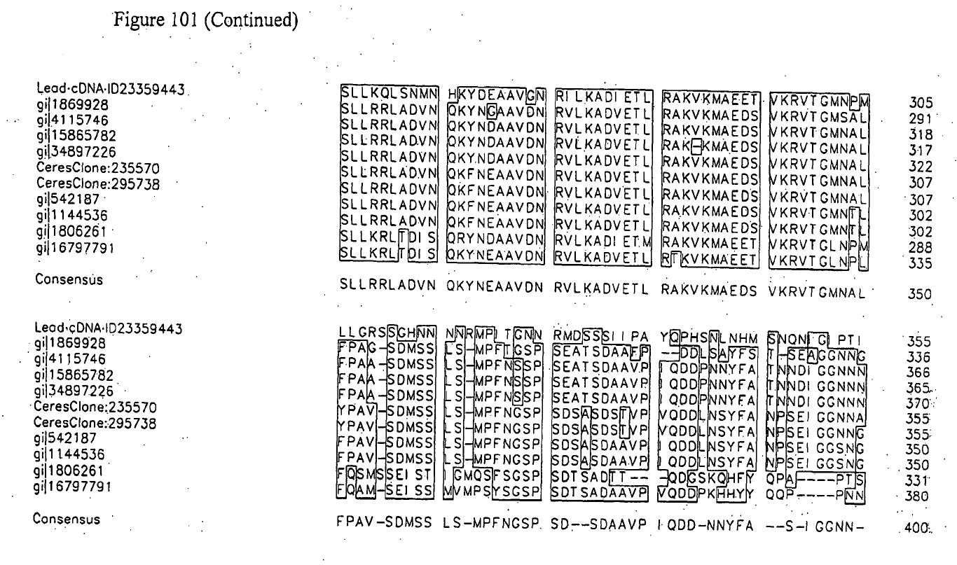

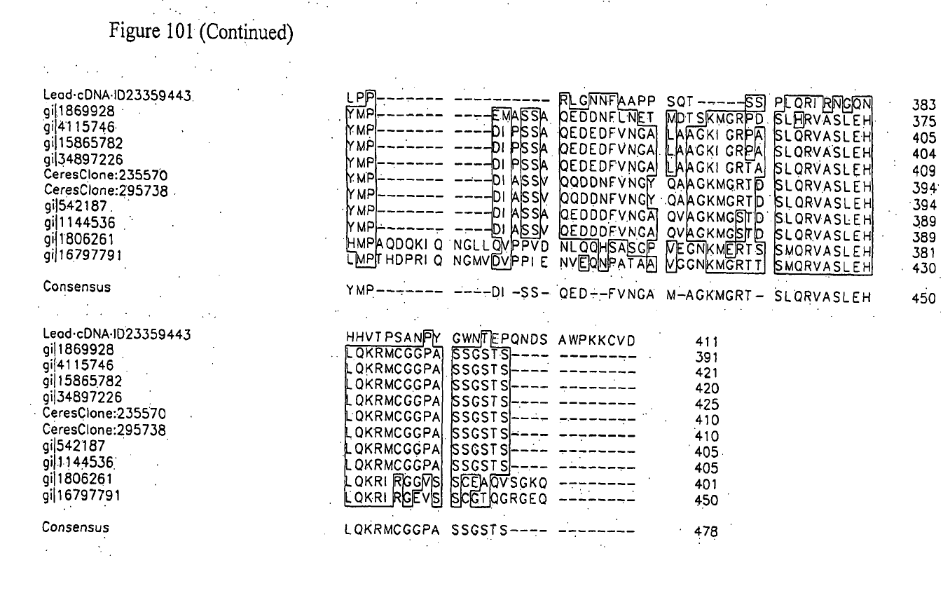

Amino acid sequences of homologs and/or orthologs of the polypeptide having the amino acid sequence set forth in SEQ ID NO:144, SEQ ID NO:565, SEQ ID NO:523, and SEQ ID NO:1026 are provided in FIG. 7, FIG. 51, FIG. 48, and FIG. 101, respectively. Each of FIG. 7, FIG. 51, FIG. 48, and FIG. 101 also includes a consensus amino acid sequence determined by aligning homologous and/or orthologous amino acid sequences with the amino acid sequence set forth in SEQ ID NO:144, SEQ ID NO:565, SEQ ID NO:523, or SEQ ID NO:1026, respectively.

For example, the alignment in FIG. 7 provides the amino acid sequences of cDNA ID 23499985 (5109F10; SEQ ID NO:144), gi|1076760 (SEQ ID NO:145), gi|1869928 (SEQ ID NO:147), CeresClone:986028 (SEQ ID NO:148), gi|12039274 (SEQ ID NO:149), and gi|463212 (SEQ ID NO:150). Other homologs and/or orthologs of SEQ ID NO:144 include Public GI no. 297482 (SEQ ID NO:146).

The alignment in FIG. 51 provides the amino acid sequences of cDNA ID 23660778 (5109A5; SEQ ID NO:565), gi|50251990 (SEQ ID NO:566), CeresClone:304939 (SEQ ID NO:567), and CeresClone:569545 (SEQ ID NO:568).

The alignment in FIG. 48 provides the amino acid sequences of cDNA ID 23357846 (SEQ ID NO:523), CeresClone:539578 (SEQ ID NO:524), CeresClone:596339 (SEQ ID NO:525), gi|6018699 (SEQ ID NO:529), and gi|50725042 (SEQ ID NO:530). Other homologs and/or orthologs of SEQ ID NO:523 include Ceres CLONE ID no. 986002 (SEQ ID NO:526), Public GI no. 2104677 (SEQ ID NO:527), and Public GI no. 23496521 (SEQ ID NO:528).

The alignment in FIG. 101 provides the amino acid sequences of cDNA ID 23359443 (SEQ ID NO:1026), gi|1806261 (SEQ ID NO:1027), gi|542187 (SEQ ID NO:1029), gi|15865782 (SEQ ID NO:1031), CeresClone:235570 (SEQ ID NO:1032), gi|16797791 (SEQ ID NO:1033), CeresClone:295738 (SEQ ID NO:1035), gi|34897226 (SEQ ID NO:1036), gi|1869928 (SEQ ID NO:1037), gi|1144536 (SEQ ID NO:1038), and gi|4115746 (SEQ ID NO:1039). Other homologs and/or orthologs of SEQ ID NO:1026 include Public GI no. 100163 (SEQ ID NO:1028), Public GI no. 168428 (SEQ ID NO:1030), Ceres CLONE ID no. 298319 (SEQ ID NO:1034), and Public GI no. 7489532 (SEQ ID NO:1040).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to SEQ ID NOs:145-150, SEQ ID NOs:566-568, SEQ ID NOs:524-530, SEQ ID NOs:1027-1040, or the consensus sequence set forth in FIG. 7, FIG. 51, FIG. 48, or FIG. 101.

A regulatory protein can have a GRAS domain characteristic of a GRAS family transcription factor. Proteins in the GRAS family are transcription factors that seem to be involved in development and other processes. For example, mutation of the SCARECROW (SCR) gene results in a radial pattern defect, loss of a ground tissue layer, in the root. The PAT1 protein is involved in phytochrome A signal transduction. GRAS proteins, such as GAI, RGA, and SCR, contain a conserved region of about 350 amino acids that can be divided into five motifs, found in the following order: the leucine heptad repeat I, the VHIID motif, the leucine heptad repeat II, the PFYRE motif, and the SAW motif. Plant specific GRAS proteins have parallels in their motif structure to the animal Signal Transducers and Activators of Transcription (STAT) family of proteins, which suggests parallels in their functions. SEQ ID NO:659 and SEQ ID NO:792 set forth the amino acid sequences of DNA clones, identified herein as cDNA ID 23515246 (SEQ ID NO:658) and cDNA ID 23365746 (SEQ ID NO:791), that are predicted to encode GRAS family transcription factor polypeptides.

A regulatory protein can comprise the amino acid sequence set forth in SEQ ID NO:659 or SEQ ID NO:792. Alternatively, a regulatory protein can be a homolog, ortholog, or variant of the polypeptide having the amino acid sequence set forth in SEQ ID NO:659 or SEQ ID NO:792. For example, a regulatory protein can have an amino acid sequence with at least 35% sequence identity, e.g., 35%, 41%, 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:659 or SEQ ID NO:792.

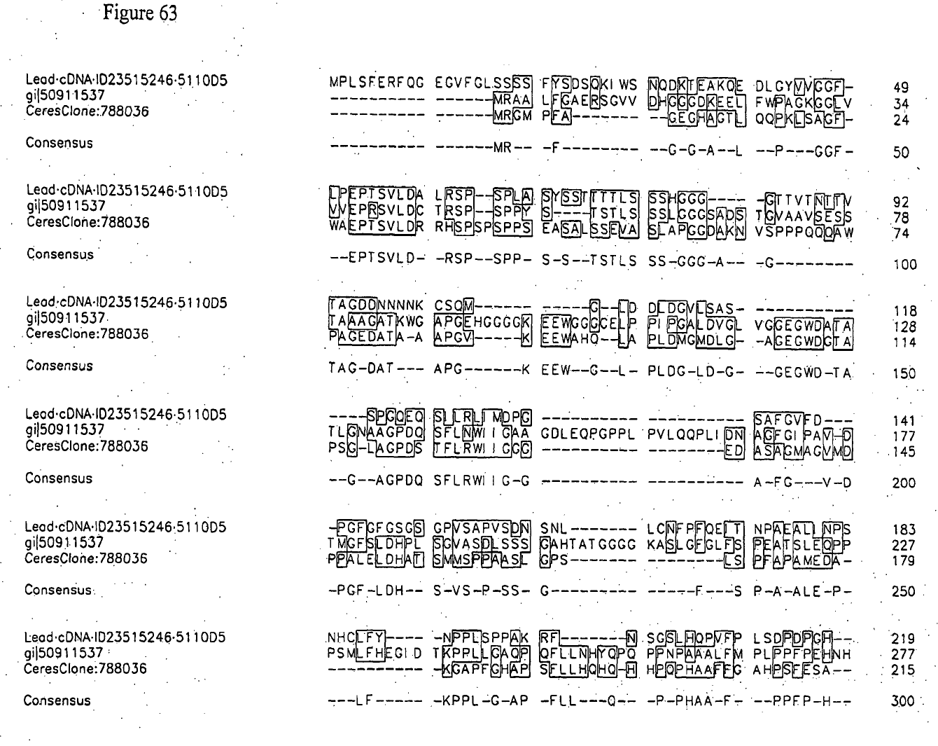

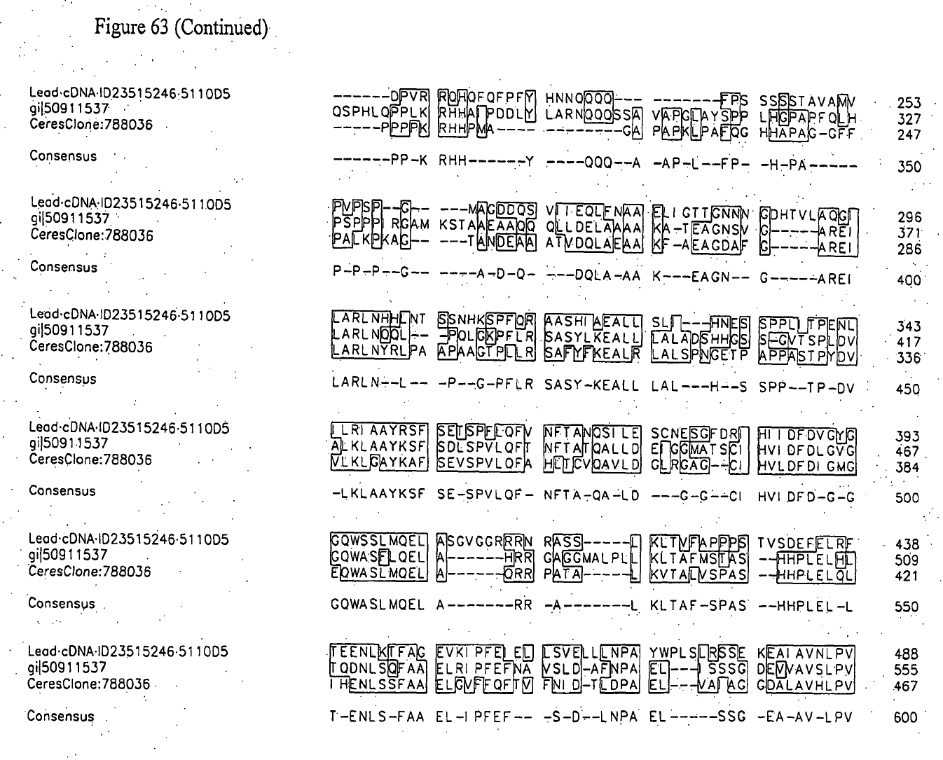

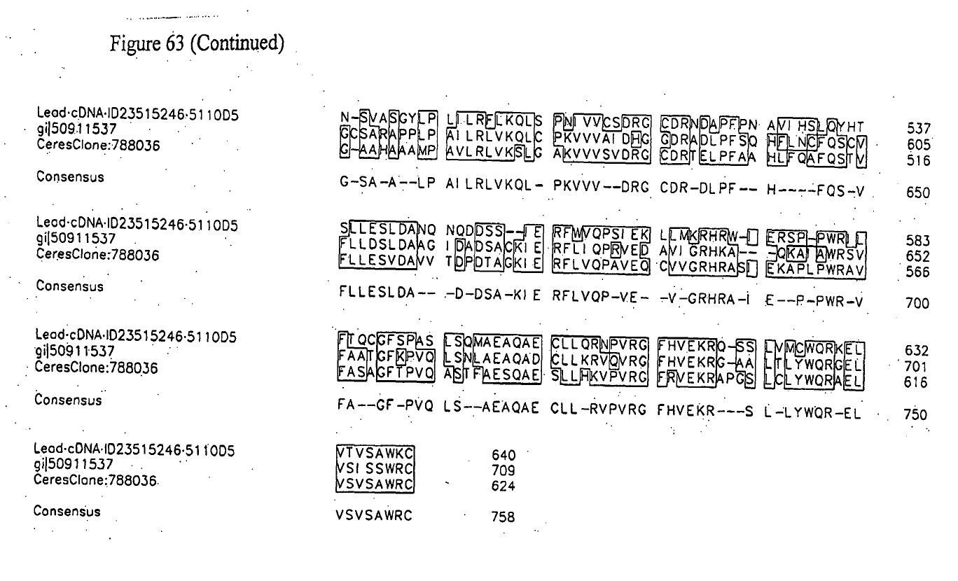

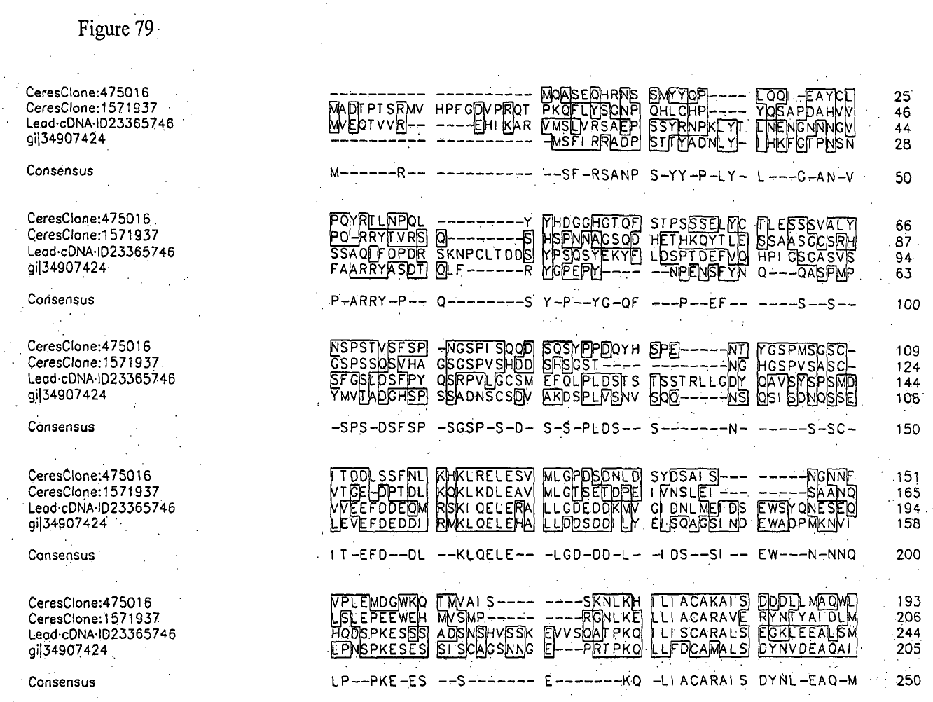

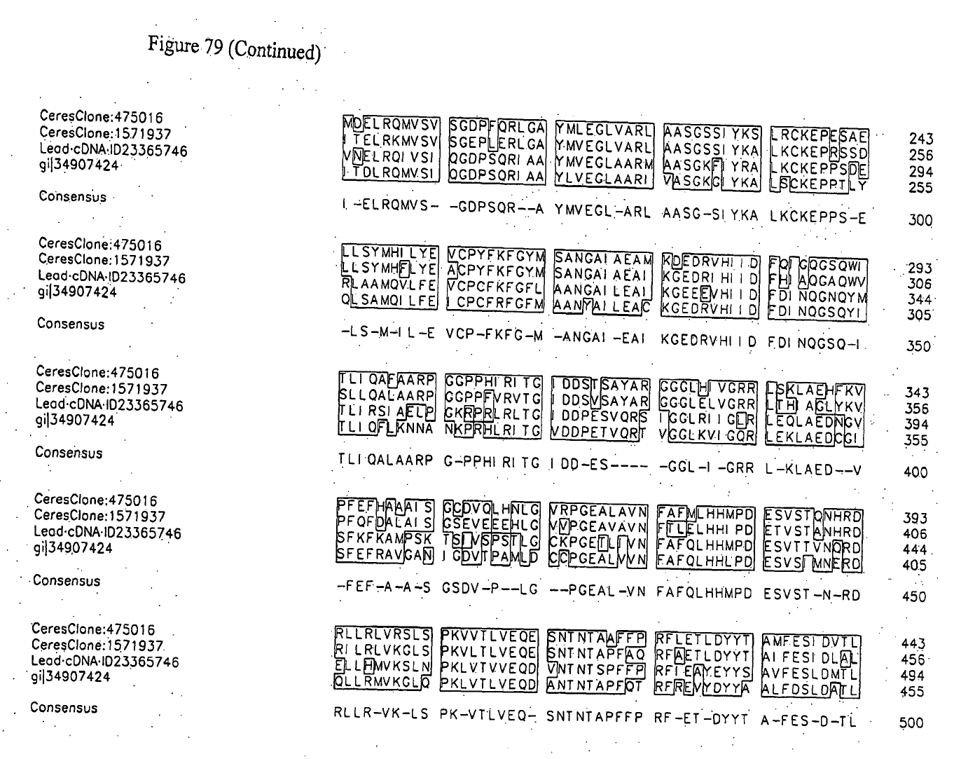

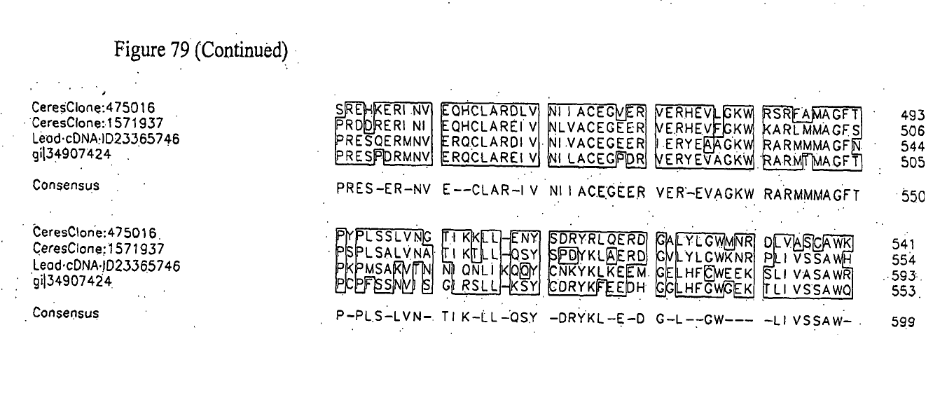

Amino acid sequences of homologs and/or orthologs of the polypeptide having the amino acid sequence set forth in SEQ ID NO:659 and SEQ ID NO:792 are provided in FIG. 63 and FIG. 79, respectively. Each of FIG. 63 and FIG. 79 also includes a consensus amino acid sequence determined by aligning homologous and/or orthologous amino acid sequences with the amino acid sequence set forth in SEQ ID NO:659 or SEQ ID NO:792, respectively.

For example, the alignment in FIG. 63 provides the amino acid sequences of cDNA ID 23515246 (5110D5; SEQ ID NO:659), gi|50911537 (SEQ ID NO:660) and CeresClone:788036 (SEQ ID NO:662). Other homologs and/or orthologs of SEQ ID NO:659 include Public GI no. 50911543 (SEQ ID NO:661).

The alignment in FIG. 79 provides the amino acid sequences of cDNA ID 23365746 (SEQ ID NO:792), gi|34907424 (SEQ ID NO:793), CeresClone:475016 (SEQ ID NO:794), and CeresClone:1571937 (SEQ ID NO:795).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:660-662, SEQ ID NOs:793-795, or the consensus sequences set forth in FIG. 63 or FIG. 79.

A regulatory protein can contain a GATA domain characteristic of a GATA zinc finger transcription factor polypeptide. A number of transcription factor polypeptides, including erythroid-specific transcription factor polypeptides and nitrogen regulatory polypeptides, specifically bind the DNA sequence (A/T)GATA(A/G) in the regulatory regions of genes. They are consequently termed GATA-binding transcription factors. The interactions occur via highly-conserved zinc finger domains in which the zinc ion is coordinated by four cysteine residues. NMR studies have shown that the core of the zinc finger comprises two irregular anti-parallel beta-sheets and an alpha-helix followed by a long loop to the C-terminal end of the finger. The N-terminus, which includes the helix, is similar in structure, but not sequence, to the N-terminal zinc module of the glucocorticoid receptor DNA binding domain. The helix and the loop connecting the two beta-sheets interact with the major groove of the DNA, while the C-terminal tail wraps around into the minor groove. It is this tail that is the essential determinant of specific binding. Interactions between the zinc finger and DNA are mainly hydrophobic, explaining the preponderance of thymines in the binding site. A large number of interactions with the phosphate backbone have also been observed. Two GATA zinc fingers are found in the GATA transcription factors. However there are several proteins which only contain a single copy of the domain. SEQ ID NO:325 and SEQ ID NO:1220 set forth the amino acid sequences of DNA clones, identified herein as cDNA ID 23420310 (SEQ ID NO:324) and cDNA ID 23527182 (SEQ ID NO:1219), respectively, that are predicted to encode GATA-binding transcription factor polypeptides.

A regulatory protein can comprise the amino acid sequence set forth in SEQ ID NO:325 or SEQ ID NO:1220. Alternatively, a regulatory protein can be a homolog, ortholog, or variant of the polypeptide having the amino acid sequence set forth in SEQ ID NO:325 or SEQ ID NO:1220. For example, a regulatory protein can have an amino acid sequence with at least 35% sequence identity, e.g., 36%, 40%, 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:325 or SEQ ID NO:1220.

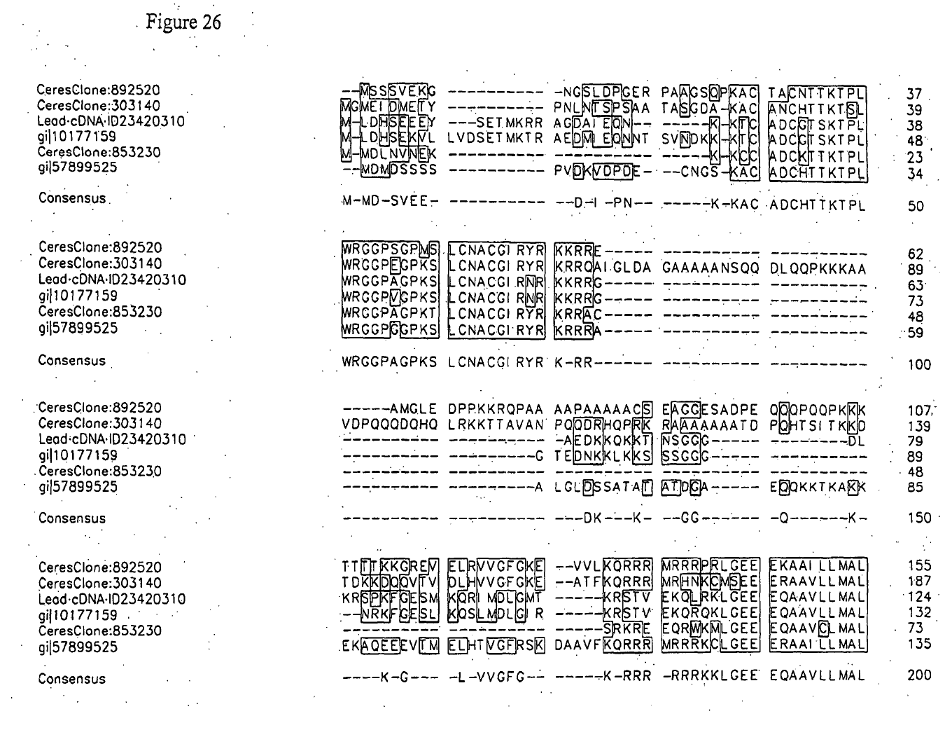

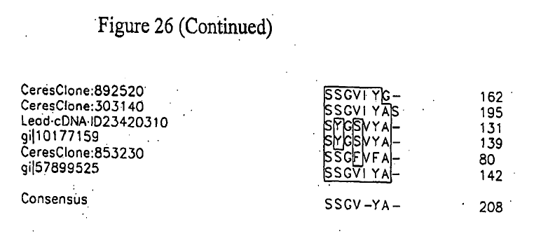

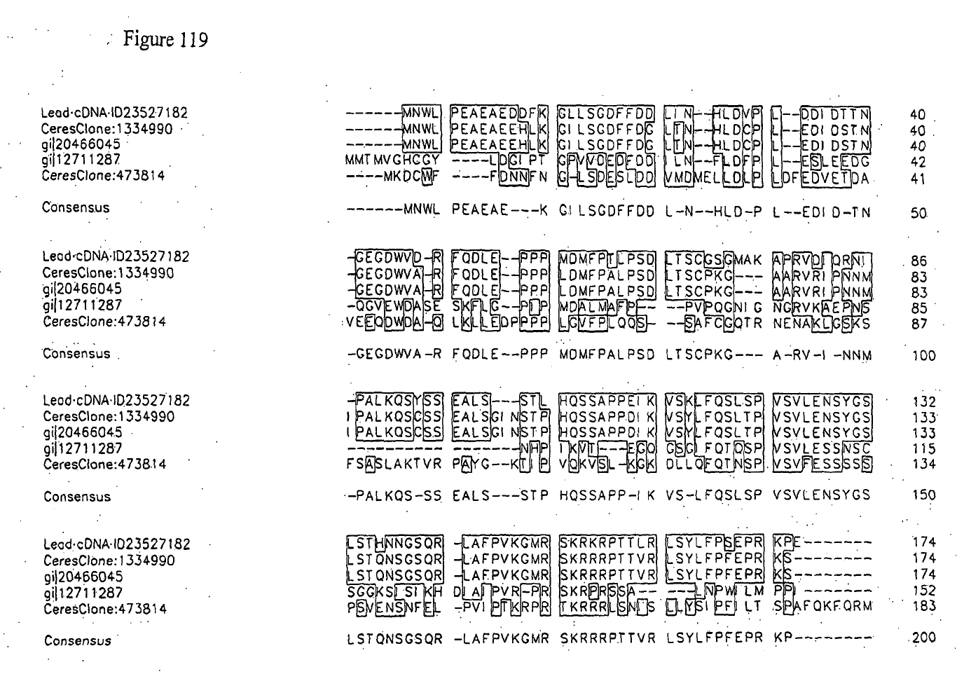

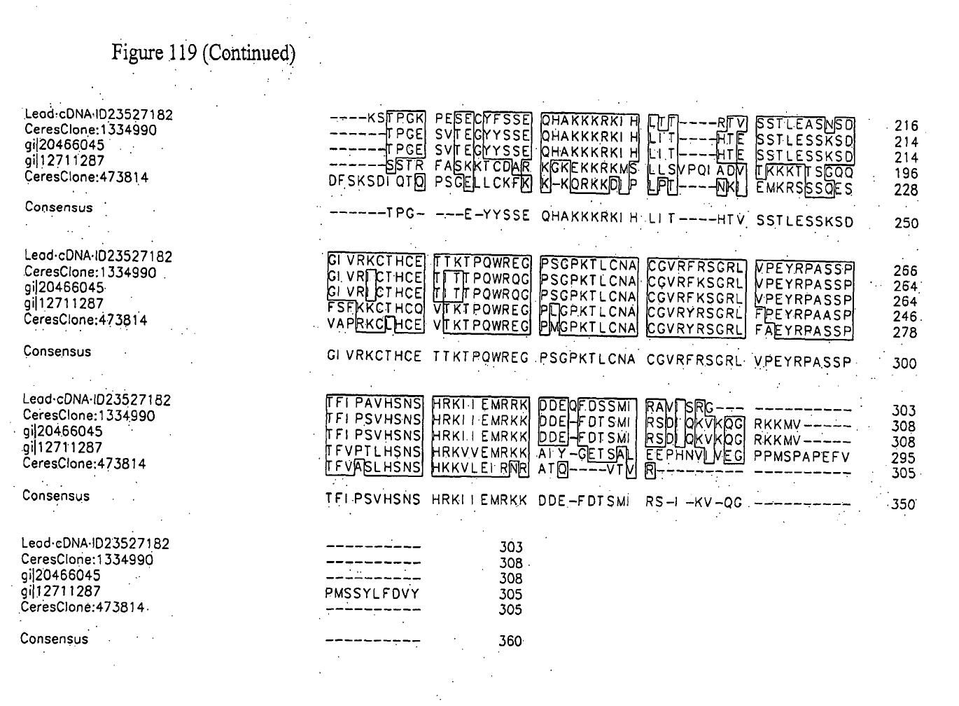

Amino acid sequences of homologs and/or orthologs of the polypeptide having the amino acid sequence set forth in SEQ ID NO:325 and SEQ ID NO:1220 are provided in FIG. 26 and FIG. 119, respectively. Each of FIG. 26 and FIG. 119 also includes a consensus amino acid sequence determined by aligning homologous and/or orthologous amino acid sequences with the amino acid sequence set forth in SEQ ID NO:325 or SEQ ID NO:1220, respectively.

For example, the alignment in FIG. 26 provides the amino acid sequences of cDNA ID 23420310 (SEQ ID NO:325), gi|10177159 (SEQ ID NO:326), CeresClone:853230 (SEQ ID NO:327), gi|57899525 (SEQ ID NO:328), CeresClone:892520 (SEQ ID NO:330), and CeresClone:303140 (SEQ ID NO:331). Other homologs and/or orthologs of SEQ ID NO:325 include Public GI no. 34897256 (SEQ ID NO:329).

The alignment in FIG. 119 provides the amino acid sequences of cDNA ID 23527182 (SEQ ID NO:1220), CeresClone:1334990 (SEQ ID NO:1221), gi|20466045 (SEQ ID NO:1222), gi|12711287 (SEQ ID NO:1223), and CeresClone:473814 (SEQ ID NO:1224).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:326-331, SEQ ID NOs:1221-1224, or the consensus sequences set forth in FIG. 26 or FIG. 119.

A regulatory protein can have an HLH (helix-loop-helix) DNA binding domain characteristic of basic-helix-loop-helix (bHLH) transcription factors. Basic-helix-loop-helix (bHLH) transcription factors belong to a family of transcriptional regulators present in three eukaryotic kingdoms. Many different functions have been identified for bHLH transcription factors in animals, including control of cell proliferation and development of specific cell lineages. In plants, bHLH transcription factors are thought to have various roles in plant cell and tissue development as well as plant metabolism. The mechanism whereby bHLH transcription factors control gene transcription often involves homo- or hetero-dimerization. There are 146 putative and bona fide bHLH genes in Arabidopsis thaliana, constituting one of the largest families of transcription factors in Arabidopsis thaliana. Comparisons with animal sequences suggest that the majority of plant bHLH genes have evolved from the ancestral group B class of bHLH genes. Twelve sub-families have been identified. Within each of these main groups, there are conserved amino acid sequence motifs outside the DNA binding domain. SEQ ID NO:364 and SEQ ID NO:856 set forth the amino acid sequences of DNA clones, identified herein as cDNA ID 23374089 (SEQ ID NO:363) and cDNA ID 23499964 (SEQ ID NO:855), respectively, each of which is predicted to encode a polypeptide having an HLH domain.

A regulatory protein can comprise the amino acid sequence set forth in SEQ ID NO:364 or SEQ ID NO:856. Alternatively, a regulatory protein can be a homolog, ortholog, or variant of the polypeptide having the amino acid sequence set forth in SEQ ID NO:364 or SEQ ID NO:856. For example, a regulatory protein can have an amino acid sequence with at least 30% sequence identity, e.g., 31%, 35%, 40%, 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:364 or SEQ ID NO:856.

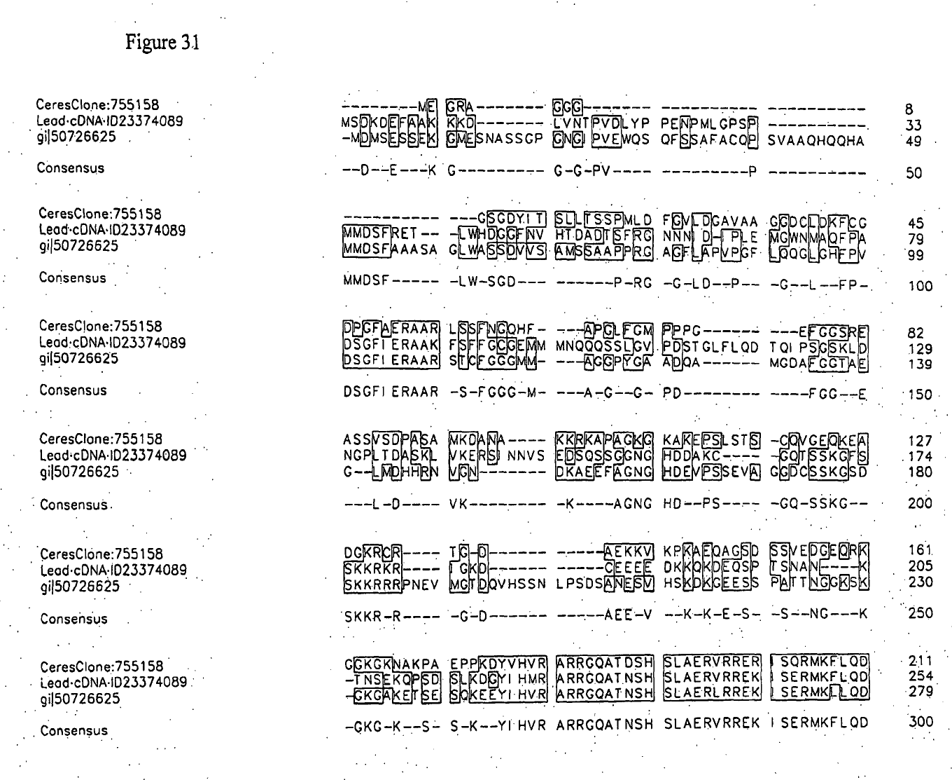

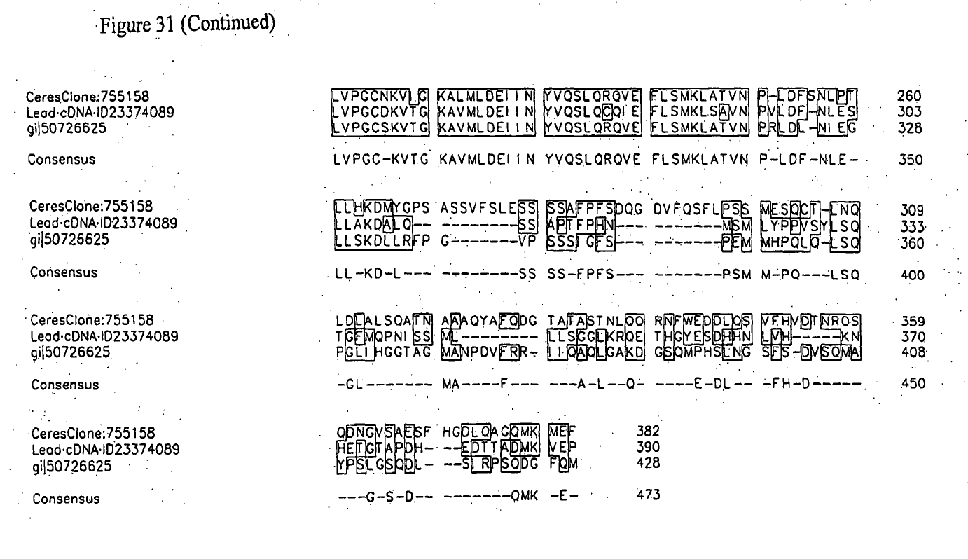

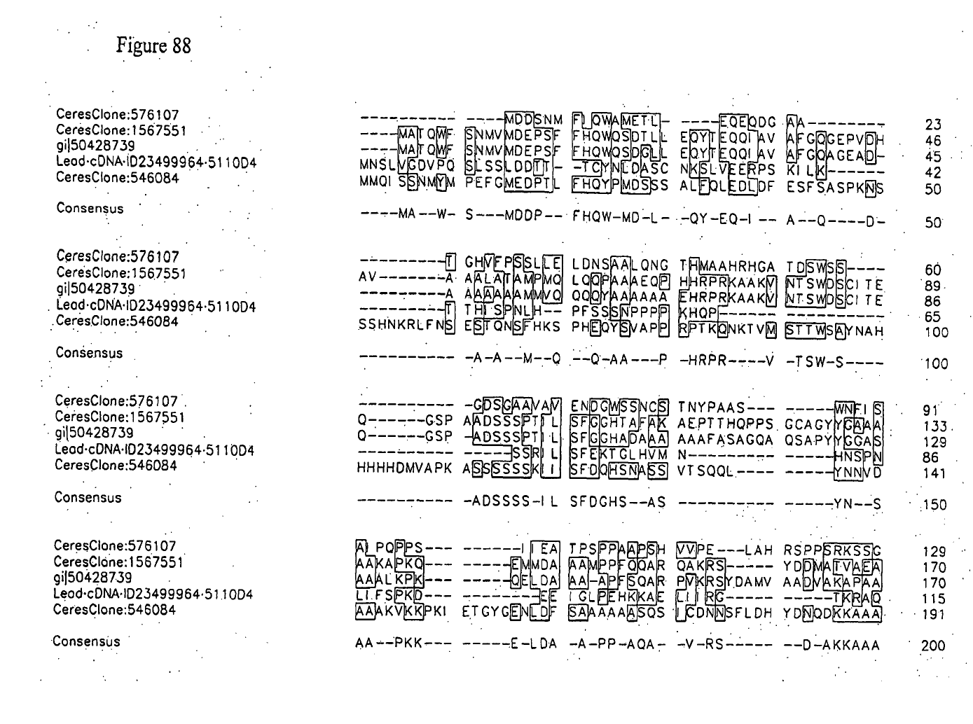

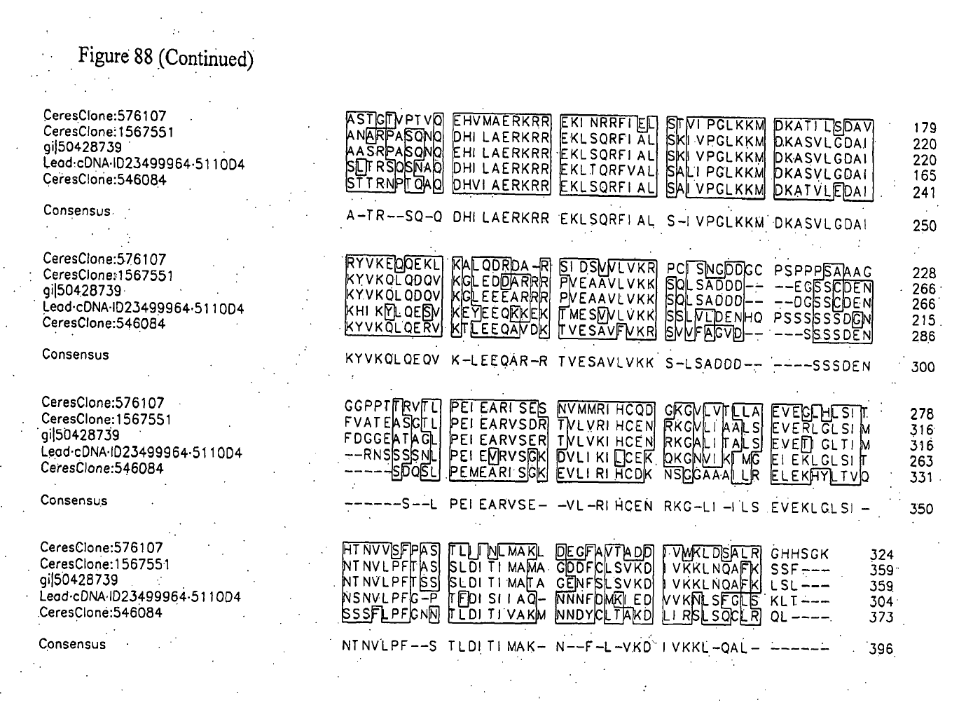

Amino acid sequences of homologs and/or orthologs of the polypeptide having the amino acid sequence set forth in SEQ ID NO:364 and SEQ ID NO:856 are provided in FIG. 31 and FIG. 88, respectively. Each of FIG. 31 and FIG. 88 also includes a consensus amino acid sequence determined by aligning homologous and/or orthologous amino acid sequences with the amino acid sequence set forth in SEQ ID NO:364 or SEQ ID NO:856, respectively.

For example, the alignment in FIG. 31 provides the amino acid sequences of cDNA ID 23374089 (SEQ ID NO:364), gi|50726625 (SEQ ID NO:365) and CeresClone:755158 (SEQ ID NO:366).

The alignment in FIG. 88 provides the amino acid sequences of cDNA ID 23499964 (5110D4; SEQ ID NO:856), CeresClone:546084 (SEQ ID NO:857), CeresClone:1567551 (SEQ ID NO:858), gi|50428739 (SEQ ID NO:859), and CeresClone:576107 (SEQ ID NO:866). Other homologs and/or orthologs of SEQ ID NO:856 include Ceres CLONE ID no. 1170120 (SEQ ID NO:860), Ceres CLONE ID no. 1603581 (SEQ ID NO:861), Ceres CLONE ID no. 536343 (SEQ ID NO:862), Ceres CLONE ID no. 526354 (SEQ ID NO:863), Ceres CLONE ID no. 478622 (SEQ ID NO:864), Ceres CLONE ID no. 472335 (SEQ ID NO:865), and Ceres CLONE ID no. 1503655 (SEQ ID NO:867).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:365-366, SEQ ID NOs:857-867, or the consensus sequences set forth in FIG. 31 or FIG. 88.

A regulatory protein can have a TCP domain characteristic of a TCP family transcription factor polypeptide. Members of the TCP family contain conserved regions that are predicted to form a non-canonical basic-helix-loop-helix (bHLP) structure. In rice, this domain was shown to be involved in DNA binding and dimerization. In Arabidopsis, members of the TCP family were expressed in rapidly growing floral primordia. It is likely that members of the TCP family affect cell division. SEQ ID NO:570 and SEQ ID NO:572 set forth the amino acid sequences of DNA clones, identified herein as cDNA ID 23493156 (SEQ ID NO:569) and cDNA ID 23518770 (SEQ ID NO:571), respectively, that are predicted to encode TCP family transcription factor polypeptides.

A regulatory protein can comprise the amino acid sequence set forth in SEQ ID NO:570 or SEQ ID NO:572. Alternatively, a regulatory protein can be a homolog, ortholog, or variant of the polypeptide having the amino acid sequence set forth in SEQ ID NO:570 or SEQ ID NO:572. For example, a regulatory protein can have an amino acid sequence with at least 30% sequence identity, e.g., 31%, 35%, 40%, 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:570 or SEQ ID NO:572.

A regulatory protein can contain an SBP domain. SBP (SQUAMOSA-PROMOTER BINDING PROTEIN) domains are found in plant polypeptides. The SBP plant polypeptide domain is a sequence specific DNA-binding domain. Polypeptides with this domain probably function as transcription factors involved in the control of early flower development. The domain contains 10 conserved cysteine and histidine residues that are likely to be zinc ligands. SEQ ID NO:450 sets forth the amino acid sequence of a DNA clone, identified herein as cDNA ID 23374668 (SEQ ID NO:449), that is predicted to encode a polypeptide containing an SBP domain.

A regulatory protein can comprise the amino acid sequence set forth in SEQ ID NO:450. Alternatively, a regulatory protein can be a homolog, ortholog, or variant of the polypeptide having the amino acid sequence set forth in SEQ ID NO:450. For example, a regulatory protein can have an amino acid sequence with at least 35% sequence identity, e.g., 35%, 40%, 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:450.

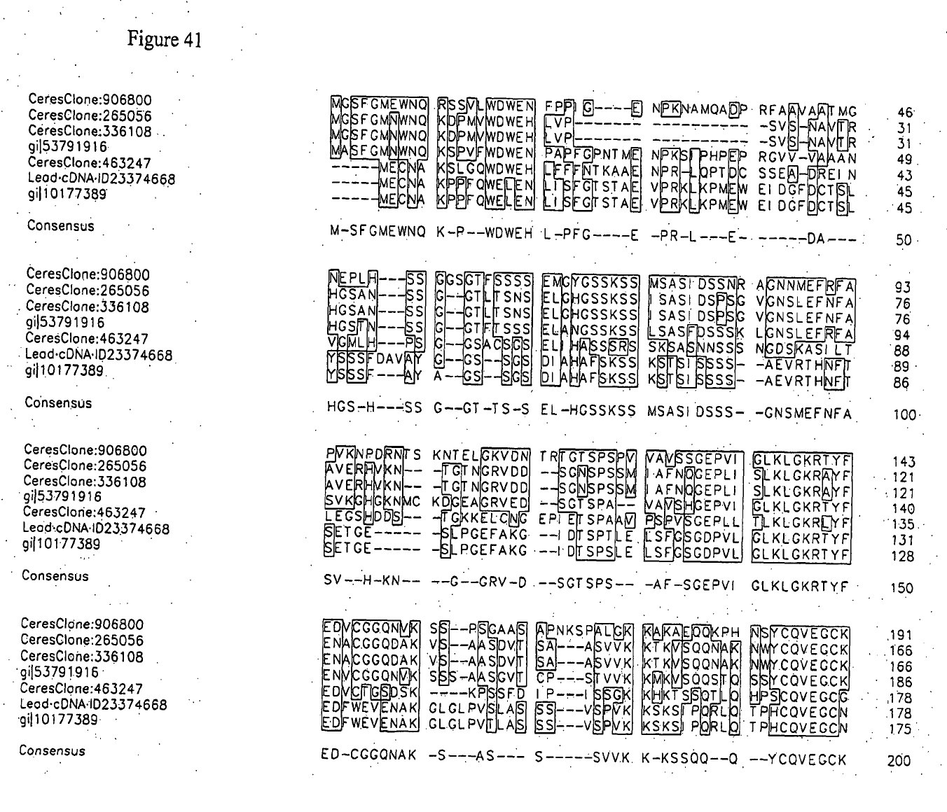

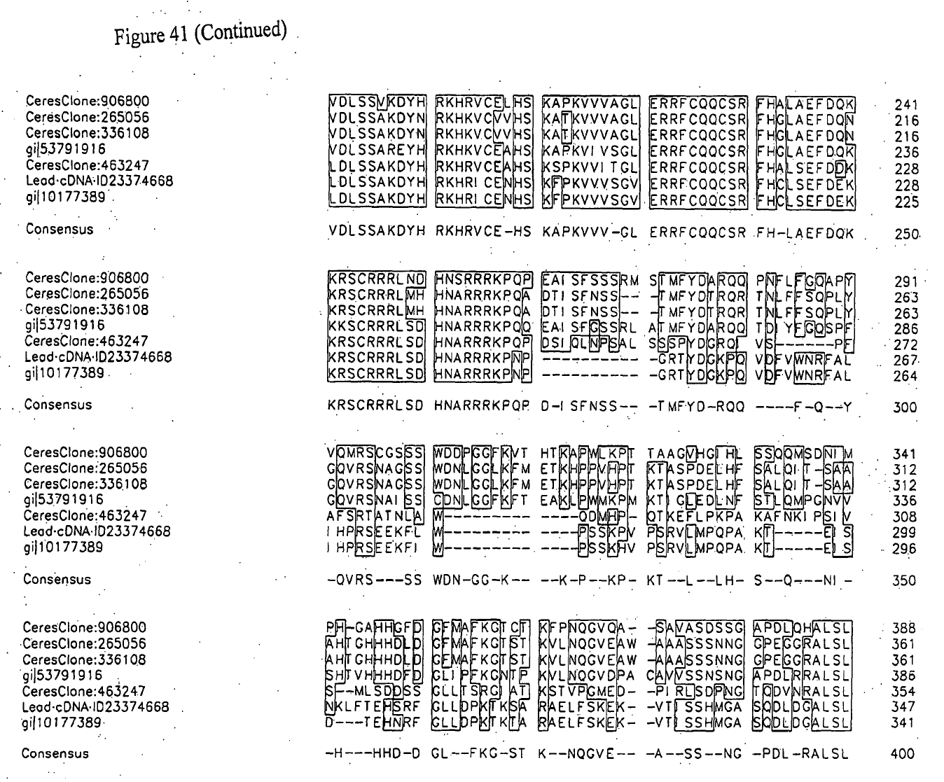

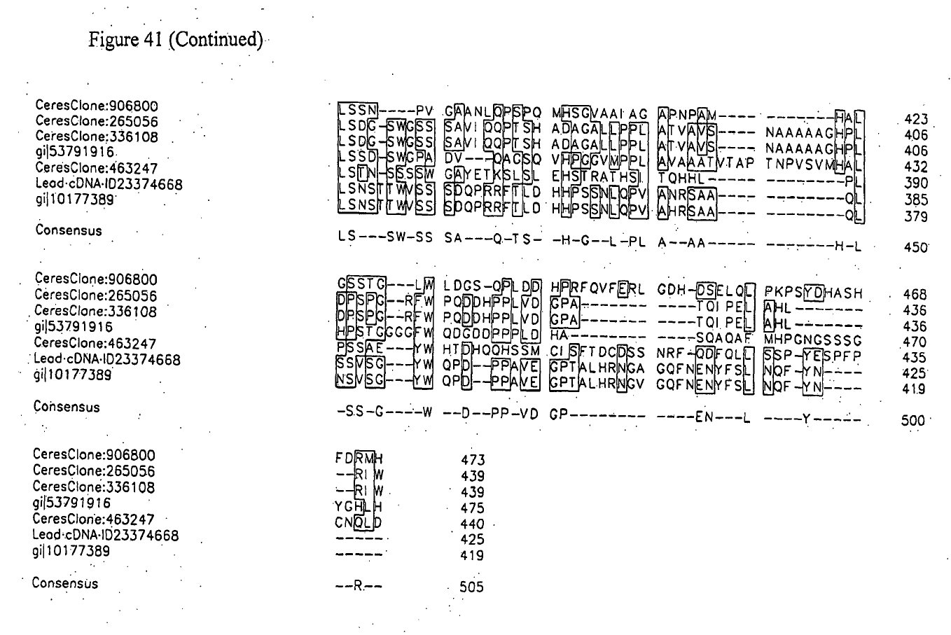

Amino acid sequences of homologs and/or orthologs of the polypeptide having the amino acid sequence set forth in SEQ ID NO:450 are provided in FIG. 41. FIG. 41 also includes a consensus amino acid sequence determined by aligning homologous and/or orthologous amino acid sequences with the amino acid sequence set forth in SEQ ID NO:450.

For example, the alignment in FIG. 41 provides the amino acid sequences of cDNA ID 23374668 (SEQ ID NO:450), gi|10177389 (SEQ ID NO:451), CeresClone:463247 (SEQ ID NO:452), gi|53791916 (SEQ ID NO:453), CeresClone:265056 (SEQ ID NO:454), CeresClone:336108 (SEQ ID NO:455), and CeresClone:906800 (SEQ ID NO:456).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:451-456 or the consensus sequence set forth in FIG. 41.

A regulatory protein can have a CBFB_NFYA domain characteristic of a CCAAT-binding transcription factor (CBF-B/NF-YA) subunit B or a CBFD_NFYB_HMF domain found in the histone-like transcription factor (CBF/NF-Y) and archaeal histones. The CCAAT-binding factor (CBFB/NF-YA) is a mammalian transcription factor that binds to a CCAAT motif in the promoters of a variety of genes, including type I collagen and albumin. The CCAAT-binding factor is a heteromeric complex of A and B subunits, both of which are required for DNA-binding. The subunits can interact in the absence of DNA-binding, with conserved regions in each subunit being important in mediating this interaction. The A subunit can be divided into three domains on the basis of sequence similarity: a non-conserved N-terminal A domain; a highly-conserved central B domain involved in DNA-binding; and a C-terminal C domain, which contains a number of glutamine and acidic residues involved in protein-protein interactions. It has been suggested that the N-terminal portion of the conserved region of the B subunit is involved in subunit interaction, while the C-terminal region of the B subunit is involved in DNA-binding. SEQ ID NO:86 sets forth the amino acid sequence of a DNA clone, identified herein as cDNA ID 23389356 (SEQ ID NO:85), that is predicted to encode a polypeptide containing a CBFB_NFYA domain. SEQ ID NO:983 sets forth the amino acid sequence of a DNA clone, identified herein as cDNA ID 23366147 (SEQ ID NO:982), that is predicted to encode a polypeptide containing a CBFD_NFYB_HMF domain.

A regulatory protein can comprise the amino acid sequence set forth in SEQ ID NO:86 or SEQ ID NO:983. Alternatively, a regulatory protein can be a homolog, ortholog, or variant of the polypeptide having the amino acid sequence set forth in SEQ ID NO:86 or SEQ ID NO:983. For example, a regulatory protein can have an amino acid sequence with at least 35% sequence identity, e.g., 35%, 40%, 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:86 or SEQ ID NO:983.

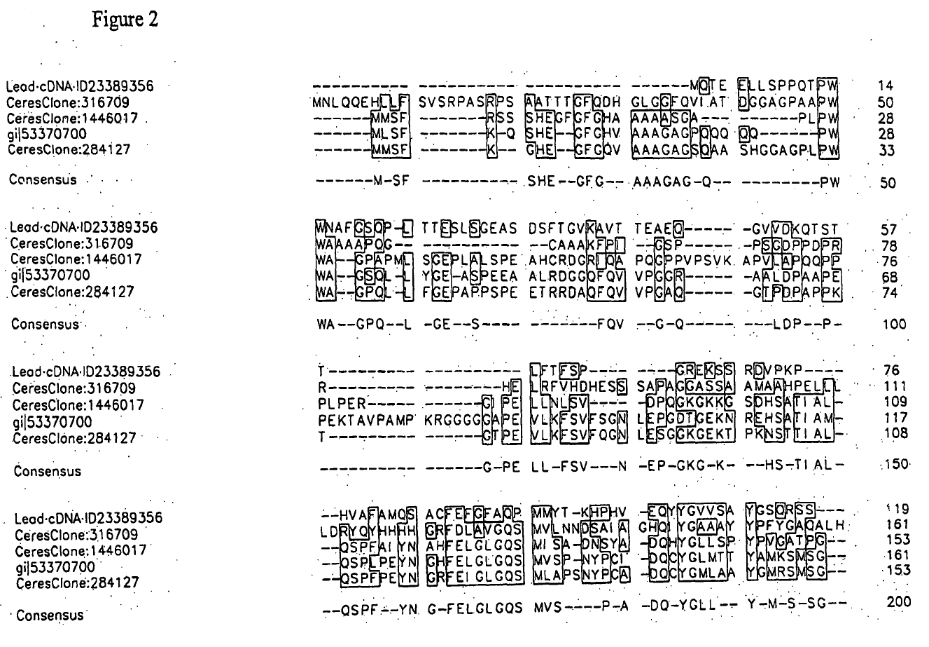

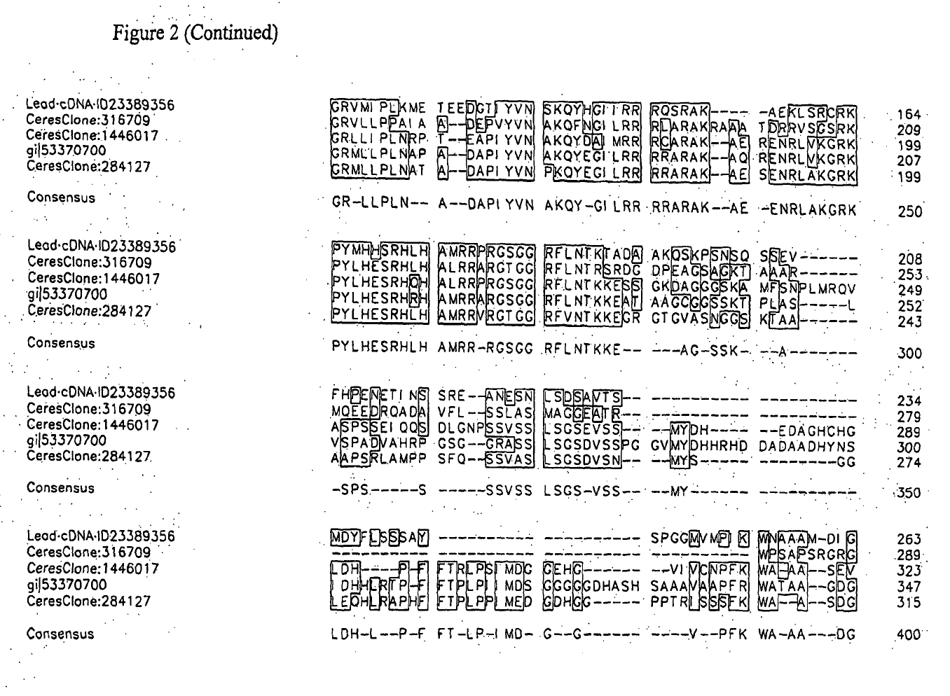

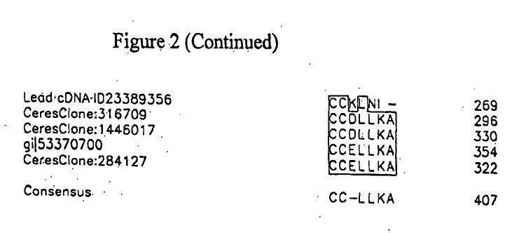

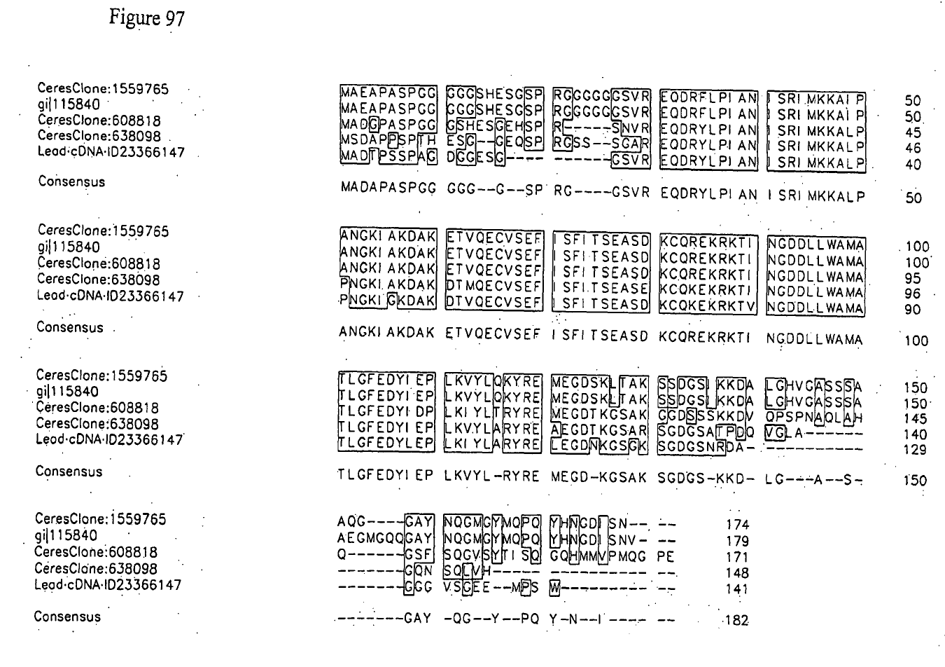

Amino acid sequences of homologs and/or orthologs of the polypeptide having the amino acid sequence set forth in SEQ ID NO:86 and SEQ ID NO:983 are provided in FIG. 2 and FIG. 97, respectively. Each of FIG. 2 and FIG. 97 also includes a consensus amino acid sequence determined by aligning homologous and/or orthologous amino acid sequences with the amino acid sequence set forth in SEQ ID NO:86 or SEQ ID NO:983, respectively.

For example, the alignment in FIG. 2 provides the amino acid sequences of cDNA ID 23389356 (SEQ ID NO:86), CeresClone:1446017 (SEQ ID NO:87), gi|53370700 (SEQ ID NO:88), CeresClone:316709 (SEQ ID NO:89), and CeresClone:284127 (SEQ ID NO:91). Other homologs and/or orthologs of SEQ ID NO:86 include Ceres CLONE ID no. 1627559 (SEQ ID NO:90).

The alignment in FIG. 97 provides the amino acid sequences of cDNA ID 23366147 (SEQ ID NO:983), CeresClone:608818 (SEQ ID NO:984), CeresClone:1559765 (SEQ ID NO:985), gi|115840 (SEQ ID NO:986), and CeresClone:638098 (SEQ ID NO:990). Other homologs and/or orthologs of SEQ ID NO:983 include Public GI no. 22380 (SEQ ID NO:987), Ceres CLONE ID no. 1561235 (SEQ ID NO:988), and Ceres CLONE ID no. 541648 (SEQ ID NO:989).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:87-91, SEQ ID NOs:984-990, or the consensus sequences set forth in FIG. 2 or FIG. 97.

A regulatory protein can have one or more domains characteristic of a homeobox polypeptide. For example, a regulatory protein can contain a homeobox domain, a HALZ domain, and a HD-ZIP_N domain. Hox genes encode homeodomain-containing transcriptional regulators that operate differential genetic programs along the anterior-posterior axis of animal bodies. The homeobox domain binds DNA through a helix-turn-helix (HTH) structure. The HTH motif is characterized by two alpha-helices, which make intimate contacts with the DNA and are joined by a short turn. The homeobox associated leucine zipper (HALZ) domain is a plant specific leucine zipper that is always found associated with a homeobox. The HD-ZIP_N domain is the N-terminus of plant homeobox-leucine zipper proteins. Homeodomain leucine zipper (HDZip) genes encode putative transcription factors that are unique to plants. SEQ ID NO:921 sets forth the amino acid sequence of a DNA clone, identified herein as cDNA ID 23385560 (SEQ ID NO:920), that is predicted to encode a polypeptide having a homeobox domain, a HALZ domain, and a HD-ZIP_N domain.

A regulatory protein can comprise the amino acid sequence set forth in SEQ ID NO:921. Alternatively, a regulatory protein can be a homolog, ortholog, or variant of the polypeptide having the amino acid sequence set forth in SEQ ID NO:921. For example, a regulatory protein can have an amino acid sequence with at least 55% sequence identity, e.g., 55%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:921.

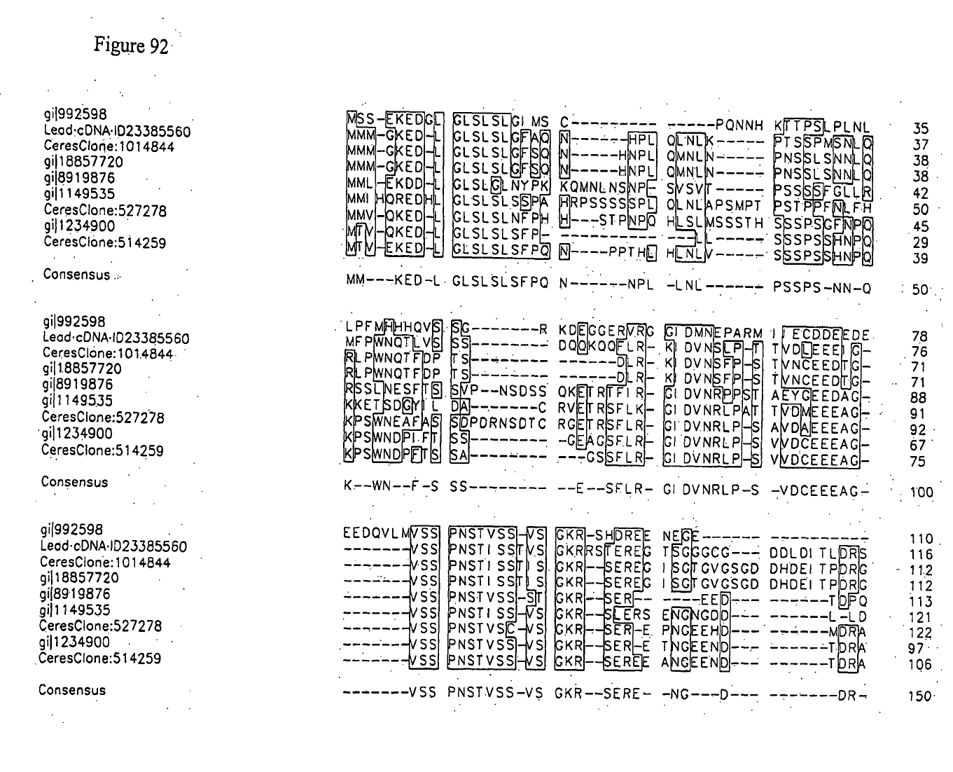

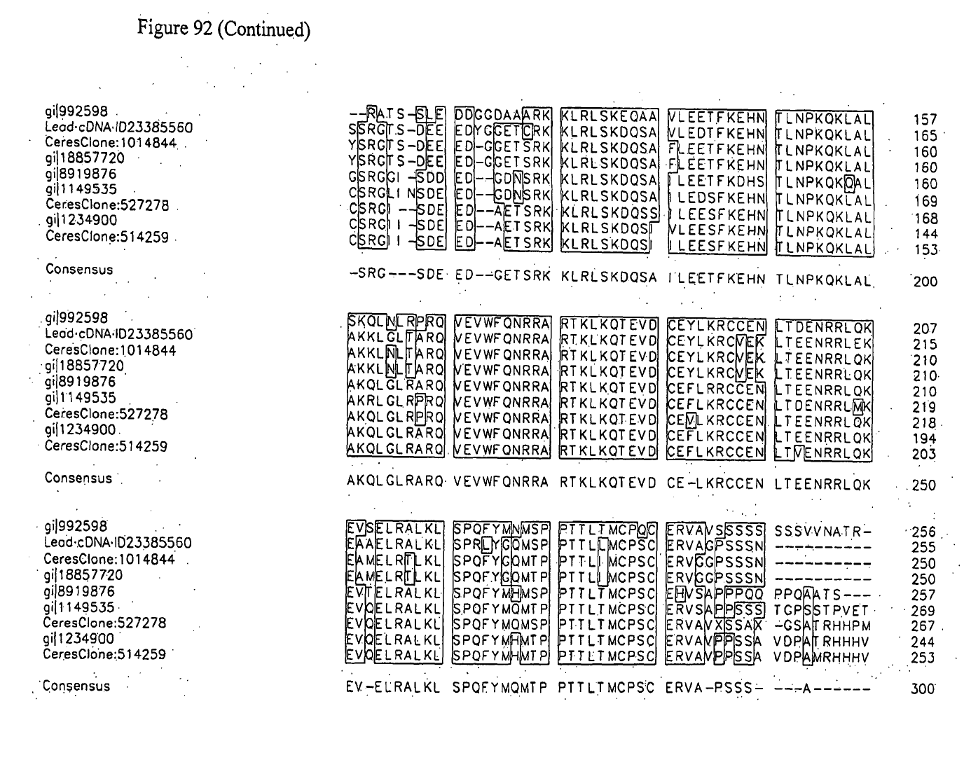

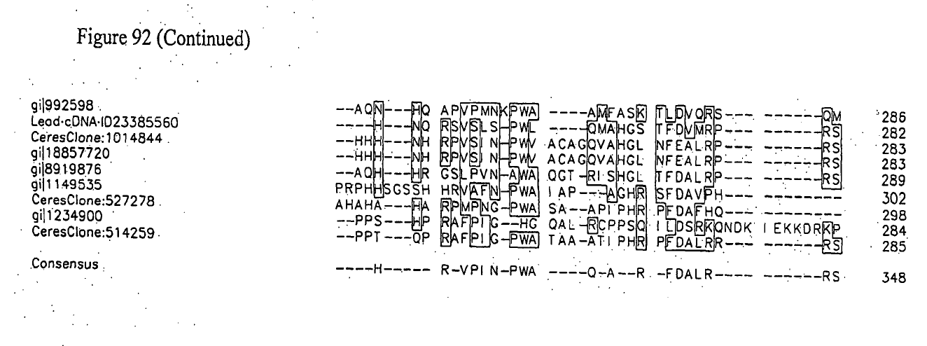

Amino acid sequences of homologs and/or orthologs of the polypeptide having the amino acid sequence set forth in SEQ ID NO:921 are provided in FIG. 92. FIG. 92 also includes a consensus amino acid sequence determined by aligning homologous and/or orthologous amino acid sequences with the amino acid sequence set forth in SEQ ID NO:921.

For example, the alignment in FIG. 92 provides the amino acid sequences of cDNA ID 23385560 (SEQ ID NO:921), CeresClone:1014844 (SEQ ID NO:922), gi|18857720 (SEQ ID NO:923), gi|1234900 (SEQ ID NO:924), CeresClone:527278 (SEQ ID NO:925), gi|1149535 (SEQ ID NO:926), CeresClone:514259 (SEQ ID NO:927), gi|8919876 (SEQ ID NO:928), and gi|992598 (SEQ ID NO:929).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:922-929 or the consensus sequence set forth in FIG. 92.

A regulatory protein can contain an HMG (high mobility group) box. HMG regulatory proteins can have one or more copies of an HMB-box motif or domain, and are involved in the regulation of DNA-dependent processes such as transcription, replication, and strand repair, all of which require the bending and unwinding of chromatin. Many of these proteins regulate gene expression. SEQ ID NO:356, SEQ ID NO:548, and SEQ ID NO:777 set forth the amino acid sequences of DNA clones, identified herein as cDNA ID 23740209 (SEQ ID NO:355), cDNA ID 23357564 (SEQ ID NO:547), and cDNA ID 23401404 (SEQ ID NO:776), respectively, each of which is predicted to encode a polypeptide containing an HMG box.

A regulatory protein can comprise the amino acid sequence set forth in SEQ ID NO:356, SEQ ID NO:548, or SEQ ID NO:777. Alternatively, a regulatory protein can be a homolog, ortholog, or variant of the polypeptide having the amino acid sequence set forth in SEQ ID NO:356, SEQ ID NO:548, or SEQ ID NO:777. For example, a regulatory protein can have an amino acid sequence with at least 35% sequence identity, e.g., 35%, 40%, 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:356, SEQ ID NO:548, or SEQ ID NO:777.

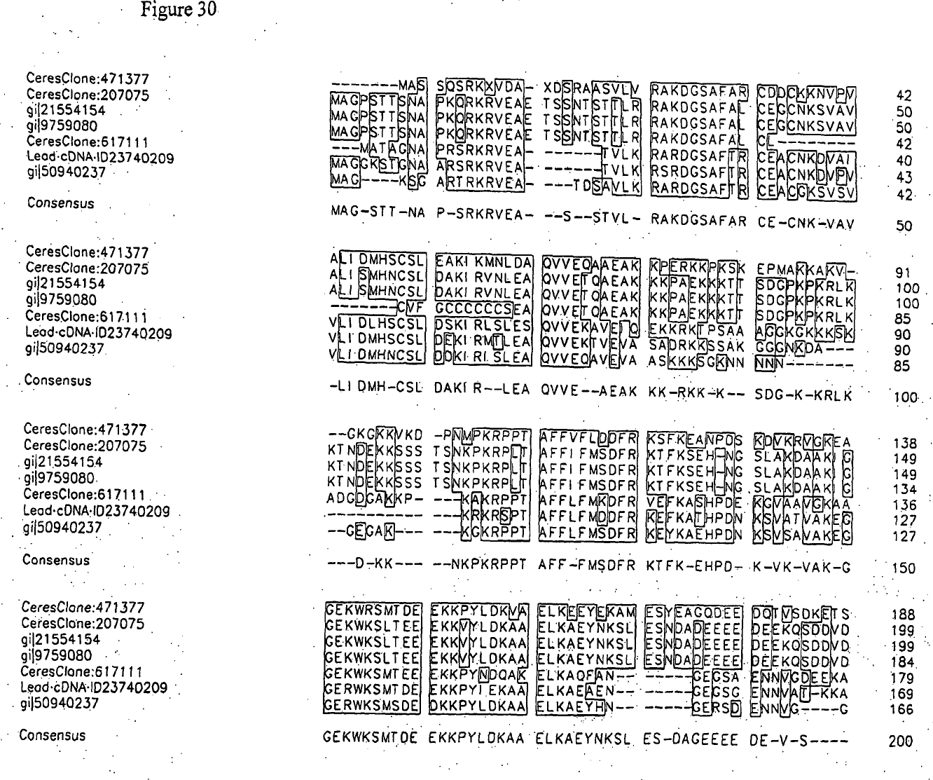

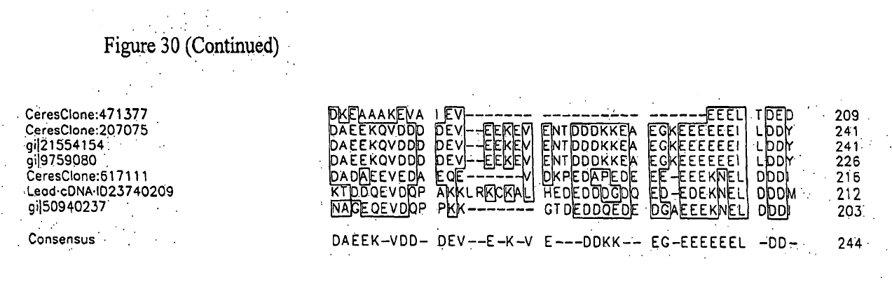

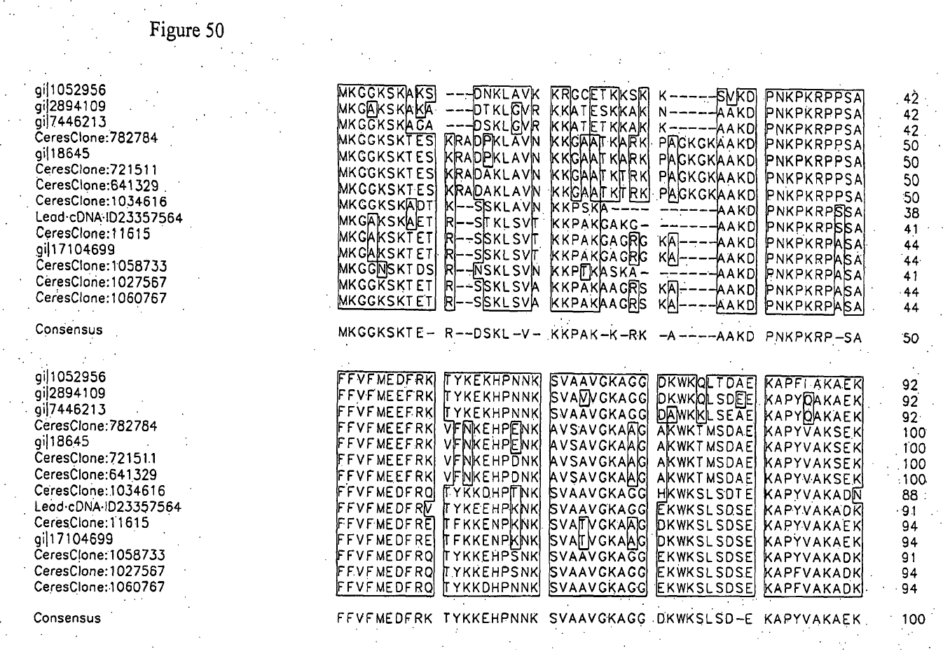

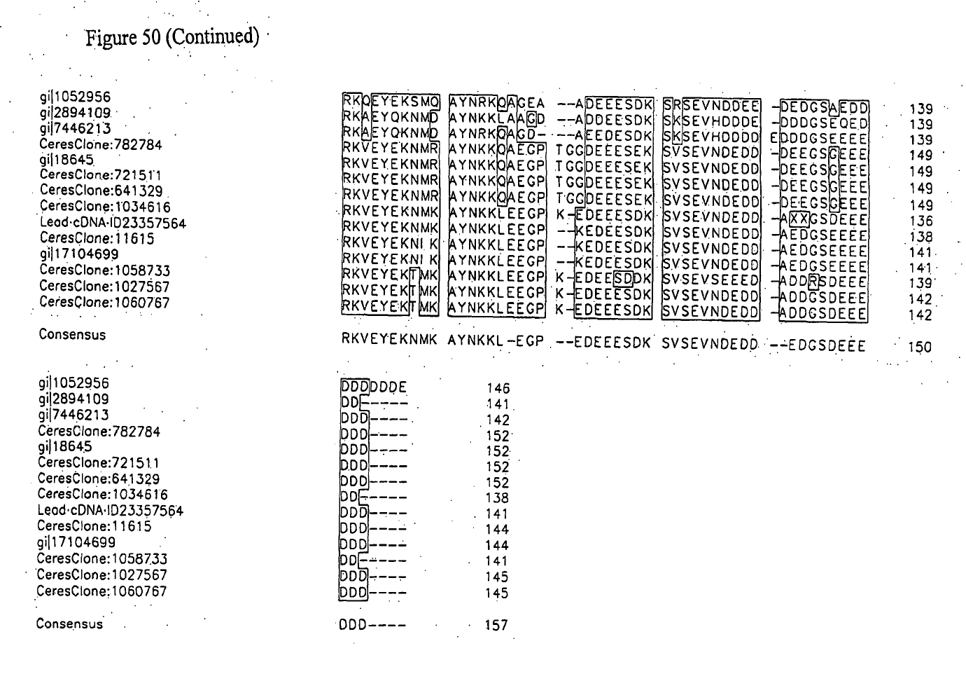

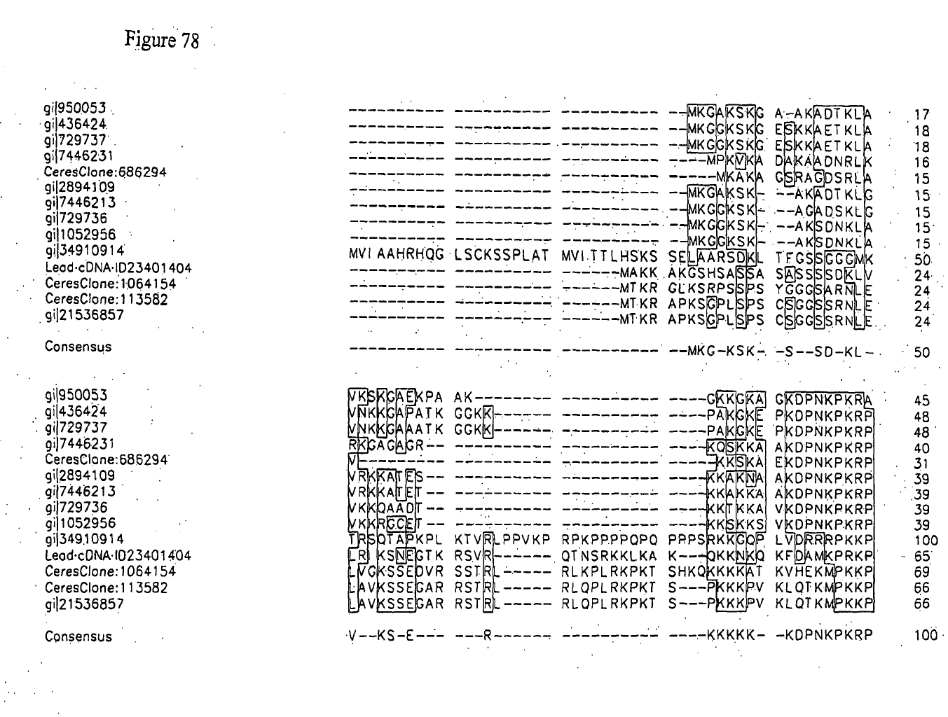

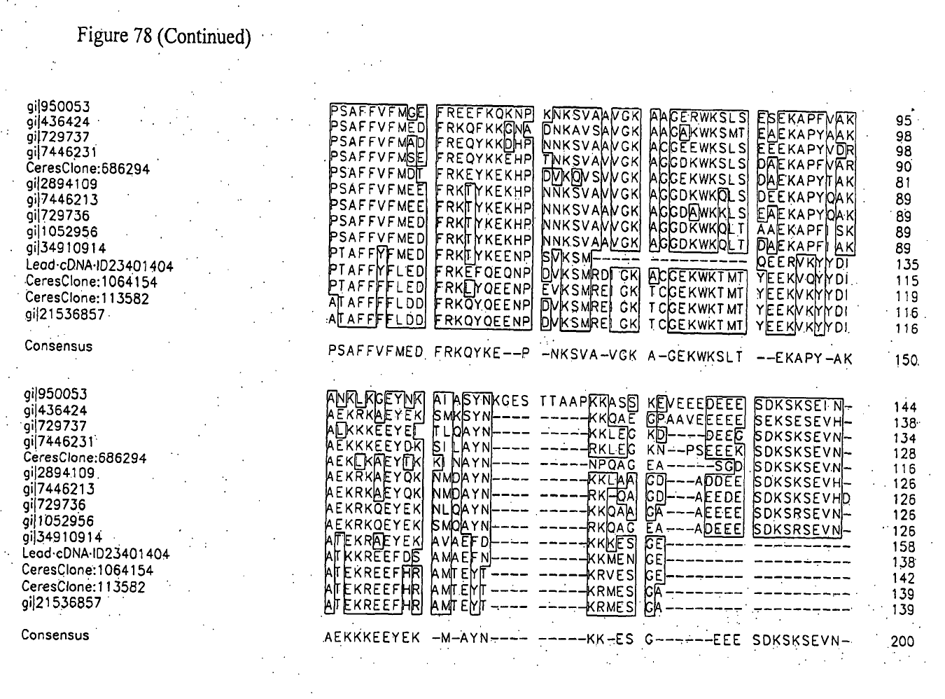

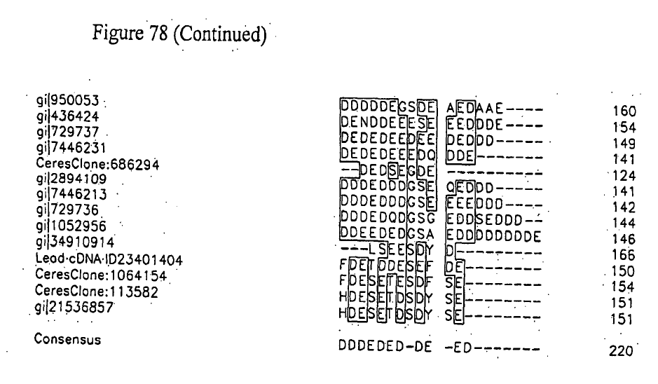

Amino acid sequences of homologs and/or orthologs of the polypeptide having the amino acid sequence set forth in SEQ ID NO:356, SEQ ID NO:548, and SEQ ID NO:777 are provided in FIG. 30, FIG. 50, and FIG. 78, respectively. Each of FIG. 30, FIG. 50, and FIG. 78 also includes a consensus amino acid sequence determined by aligning homologous and/or orthologous amino acid sequences with the amino acid sequence set forth in SEQ ID NO:356, SEQ ID NO:548, or SEQ ID NO:777, respectively.

For example, the alignment in FIG. 30 provides the amino acid sequences of cDNA ID 23740209 (SEQ ID NO:356), gi|50940237 (SEQ ID NO:357), CeresClone:617111 (SEQ ID NO:358), CeresClone:207075 (SEQ ID NO:359), gi|21554154 (SEQ ID NO:360), gi|9759080 (SEQ ID NO:361), and CeresClone:471377 (SEQ ID NO:362).

The alignment in FIG. 50 provides the amino acid sequences of cDNA ID 23357564 (SEQ ID NO:548), CeresClone:11615 (SEQ ID NO:549), gi|17104699 (SEQ ID NO:550), CeresClone:1027567 (SEQ ID NO:551), CeresClone:1060767 (SEQ ID NO:552), CeresClone:1034616 (SEQ ID NO:553), CeresClone:1058733 (SEQ ID NO:554), gi|2894109 (SEQ ID NO:555), CeresClone:782784 (SEQ ID NO:556), gi|18645 (SEQ ID NO:557), CeresClone:721511 (SEQ ID NO:558), CeresClone:641329 (SEQ ID NO:559), gi|7446213 (SEQ ID NO:560), and gi|1052956 (SEQ ID NO:561).

The alignment in FIG. 78 provides the amino acid sequences of cDNA ID 23401404 (SEQ ID NO:777), gi|34910914 (SEQ ID NO:778), CeresClone:1064154 (SEQ ID NO:779), CeresClone:113582 (SEQ ID NO:780), gi|21536857 (SEQ ID NO:781), gi|2894109 (SEQ ID NO:782), CeresClone:686294 (SEQ ID NO:783), gi|436424 (SEQ ID NO:784), gi|950053 (SEQ ID NO:785), gi|7446213 (SEQ ID NO:786), gi|729737 (SEQ ID NO:787), gi|7446231 (SEQ ID NO:788), gi|729736 (SEQ ID NO:789), and gi|1052956 (SEQ ID NO:790).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:357-362, SEQ ID NOs:549-561, SEQ ID NOs:778-790, or the consensus sequences set forth in FIG. 30, FIG. 50, or FIG. 78.

A regulatory protein can have a NAM domain characteristic of a No apical meristem (NAM) polypeptide. No apical meristem (NAM) polypeptides are plant development polypeptides. NAM is indicated as having a role in determining positions of meristems and primordia. The NAC domain (NAM for Petunia hybrida and ATAF1, ATAF2, and CUC2 for Arabidopsis) is an N-terminal module of about 160 amino acids, which is found in proteins of the NAC family of plant-specific transcriptional regulators (no apical meristem polypeptides). NAC proteins are involved in developmental processes, including formation of the shoot apical meristem, floral organs and lateral shoots, as well as in plant hormonal control and defense. The NAC domain is accompanied by diverse C-terminal transcriptional activation domains. The NAC domain has been shown to be a DNA-binding domain (DBD) and a dimerization domain. SEQ ID NO:419, SEQ ID NO:579, and SEQ ID NO:1310 set forth the amino acid sequences of DNA clones, identified herein as cDNA ID 23382112 (SEQ ID NO:417), cDNA ID 23467847 (SEQ ID NO:578), and cDNA ID 23396143 (SEQ ID NO:1309), respectively.

A regulatory protein can comprise the amino acid sequence set forth in SEQ ID NO:419, SEQ ID NO:579, or SEQ ID NO:1310. Alternatively, a regulatory protein can be a homolog, ortholog, or variant of the polypeptide having the amino acid sequence set forth in SEQ ID NO:419, SEQ ID NO:579, or SEQ ID NO:1310. For example, a regulatory protein can have an amino acid sequence with at least 35% sequence identity, e.g., 35%, 40%, 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:419, SEQ ID NO:579, or SEQ ID NO:1310.

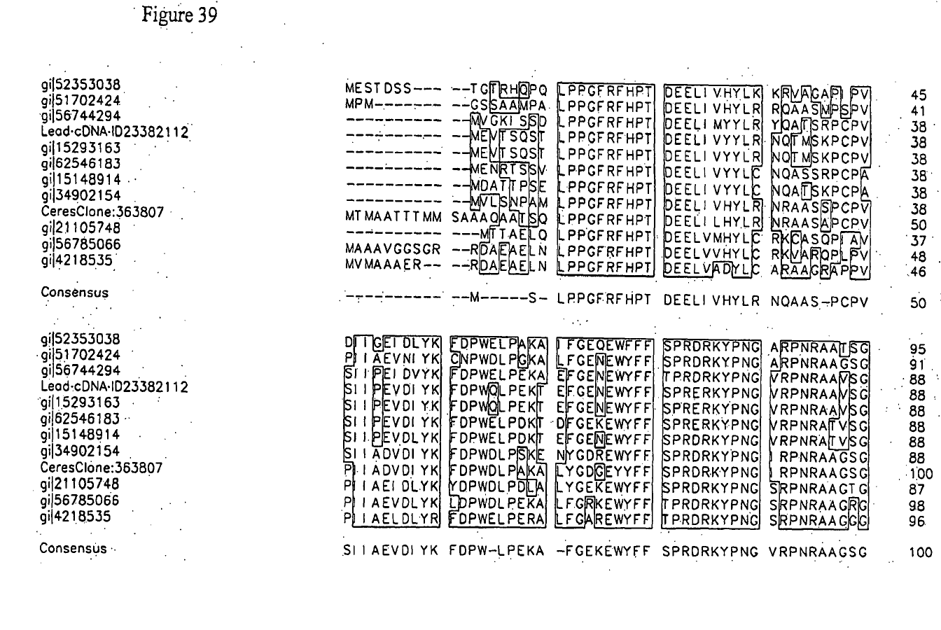

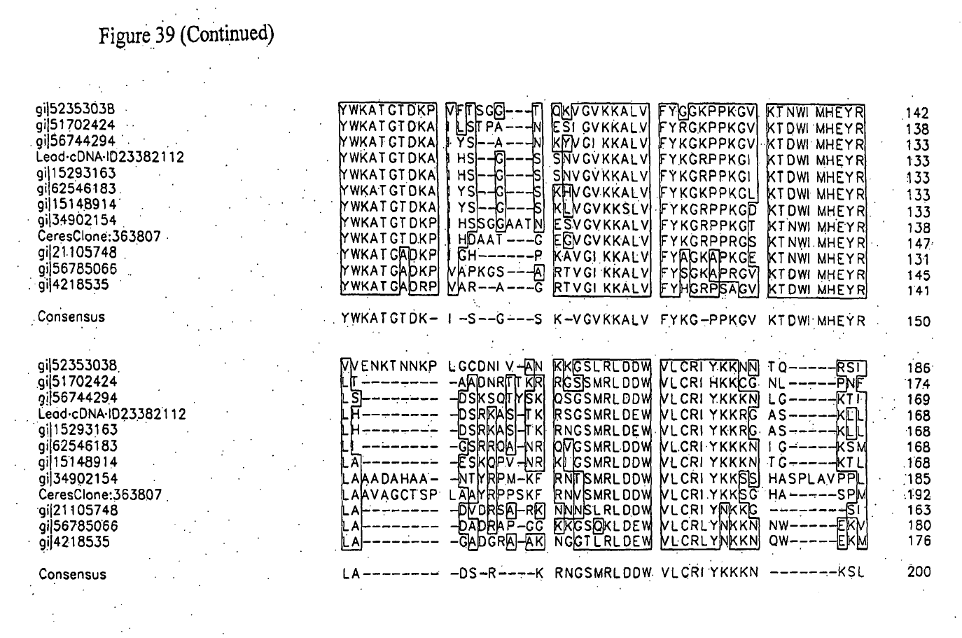

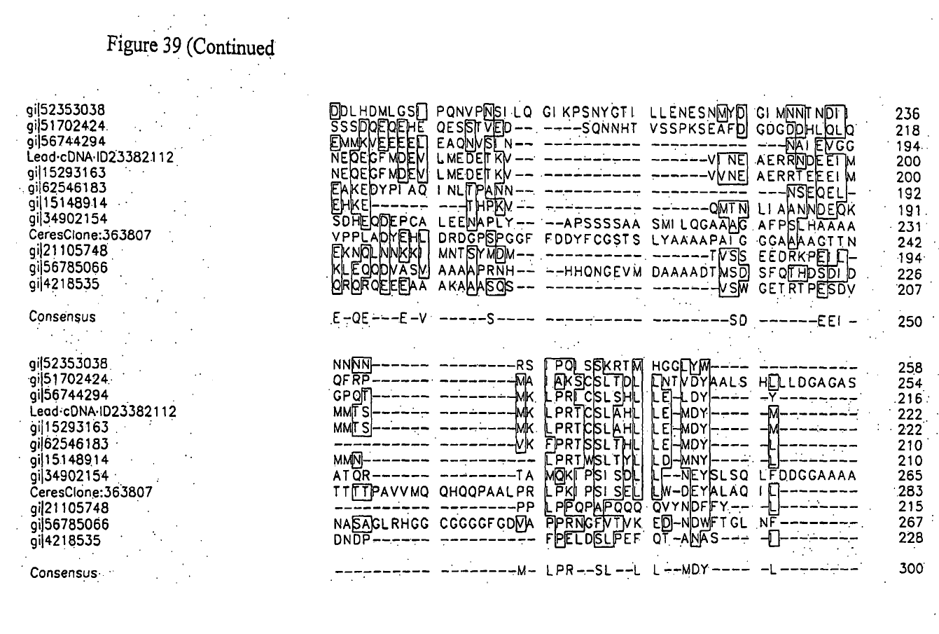

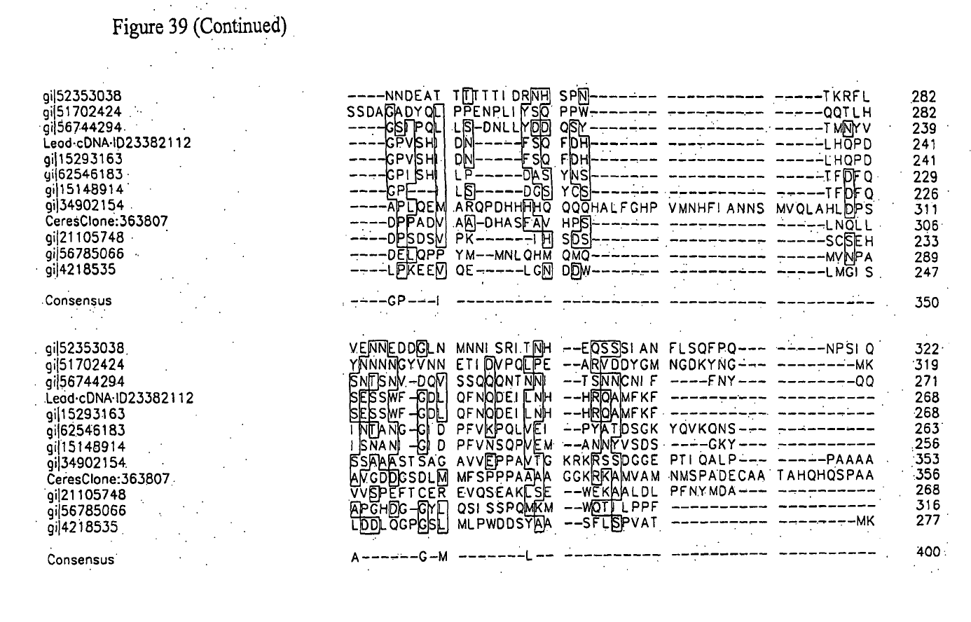

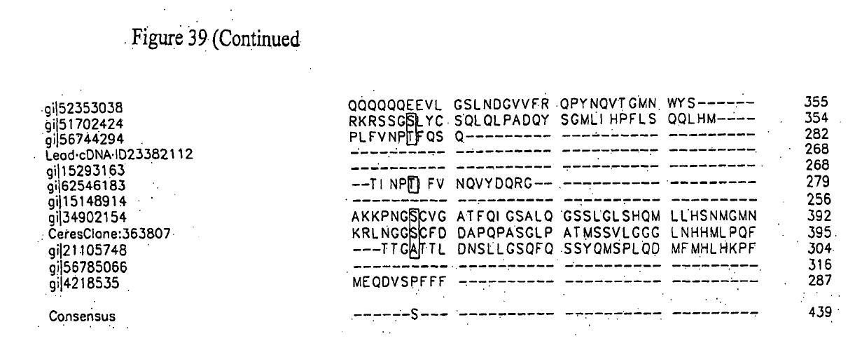

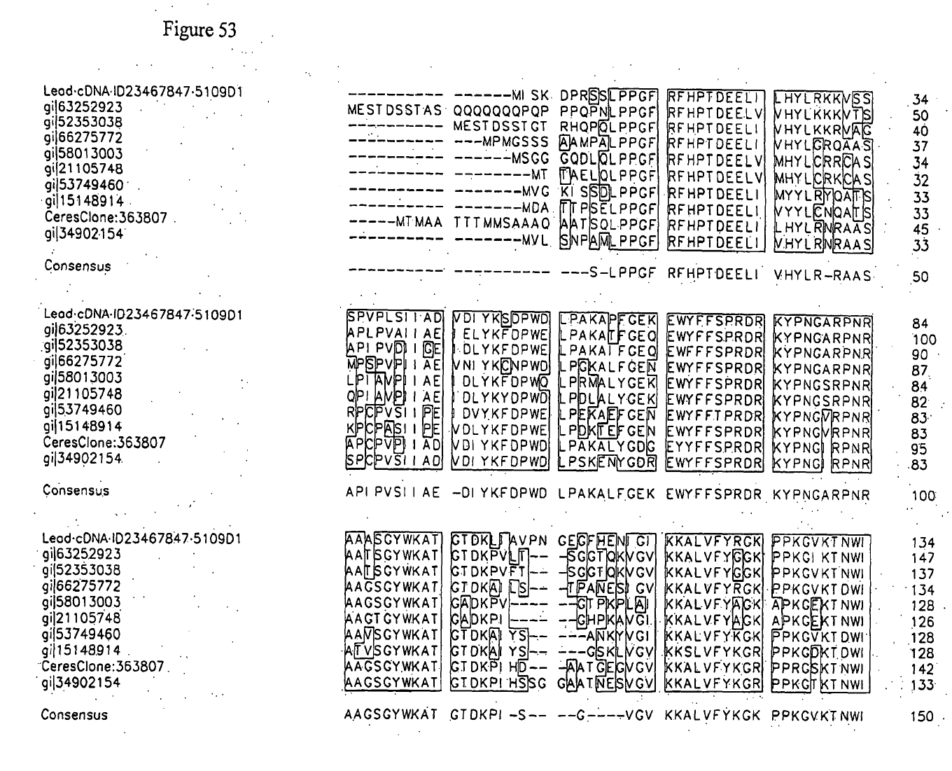

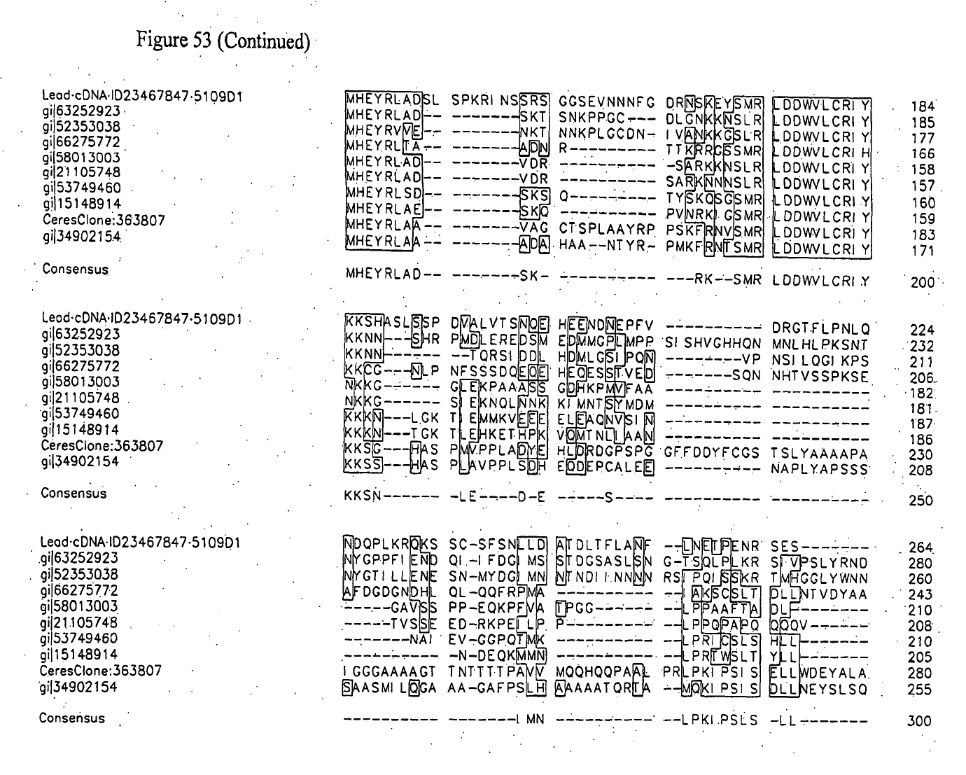

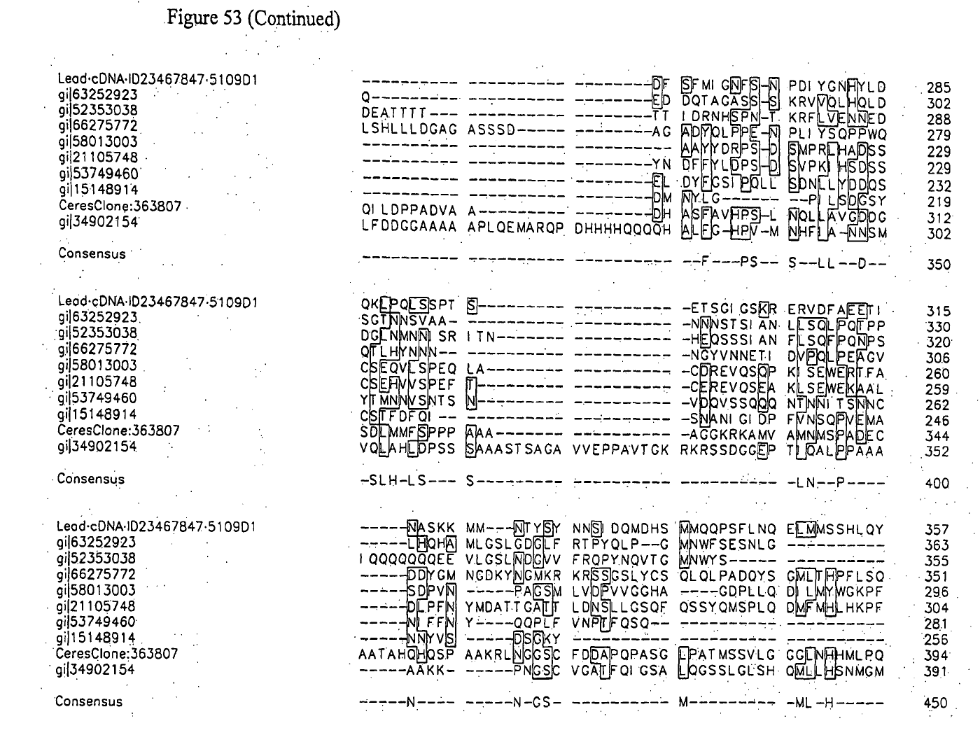

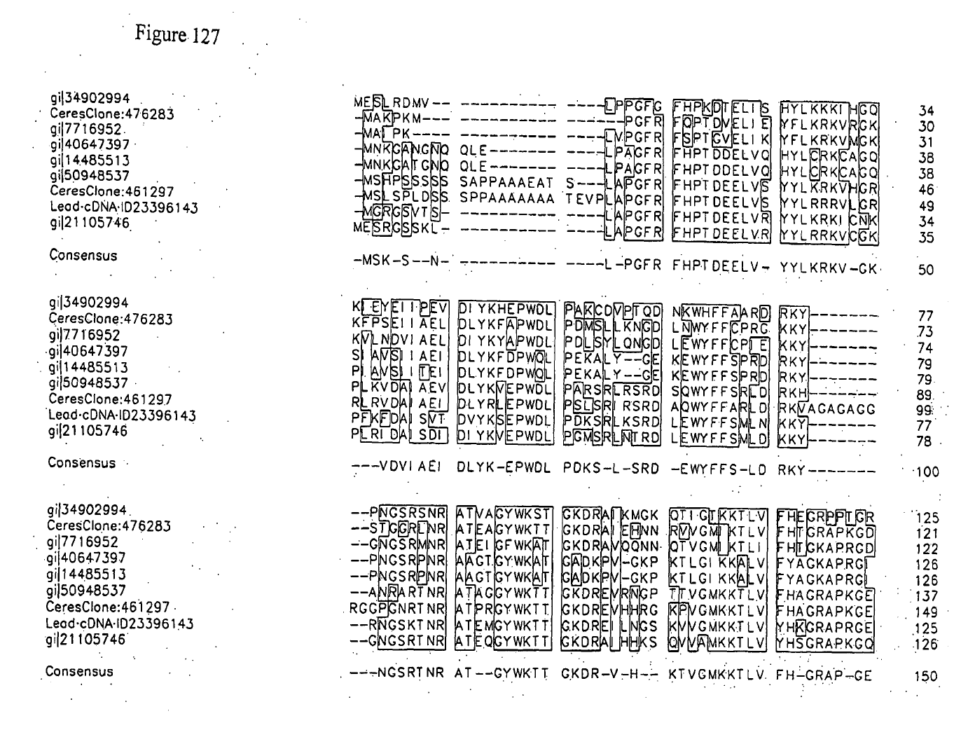

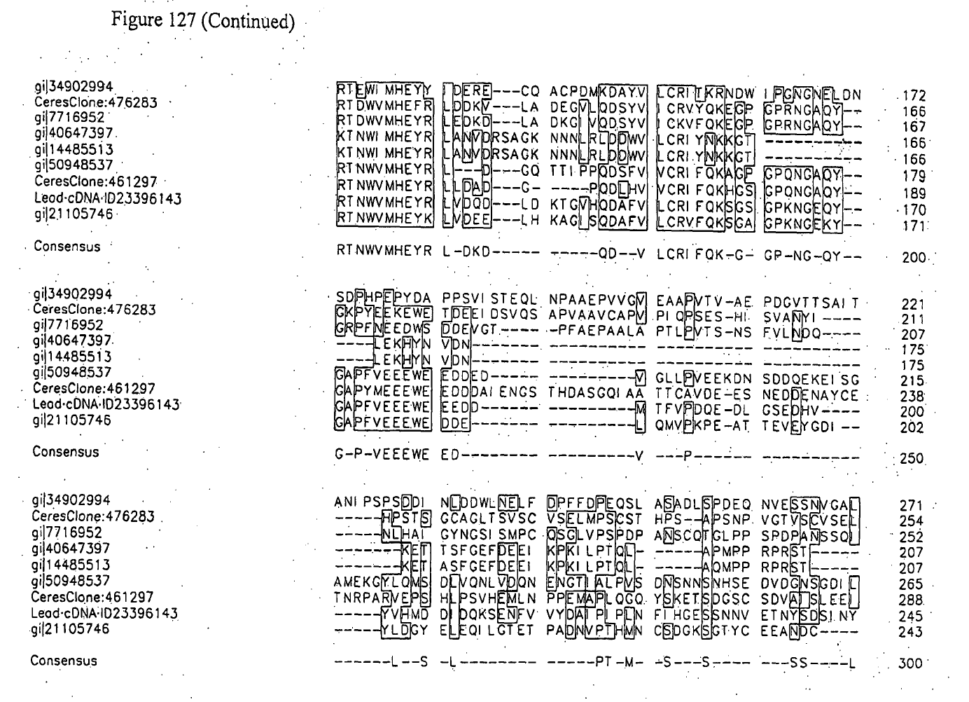

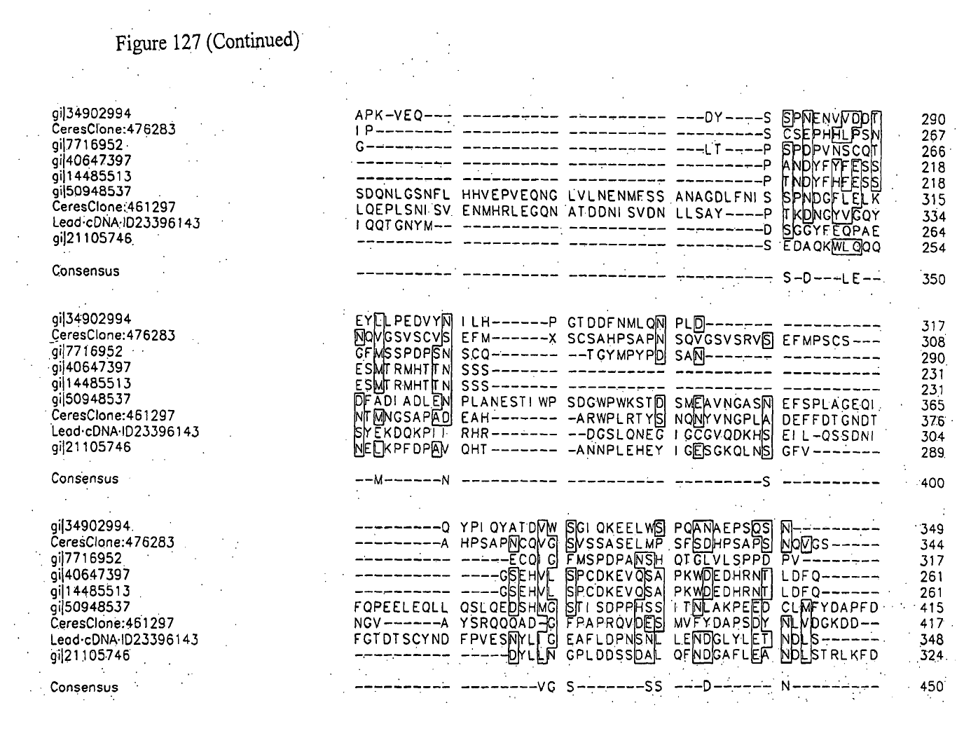

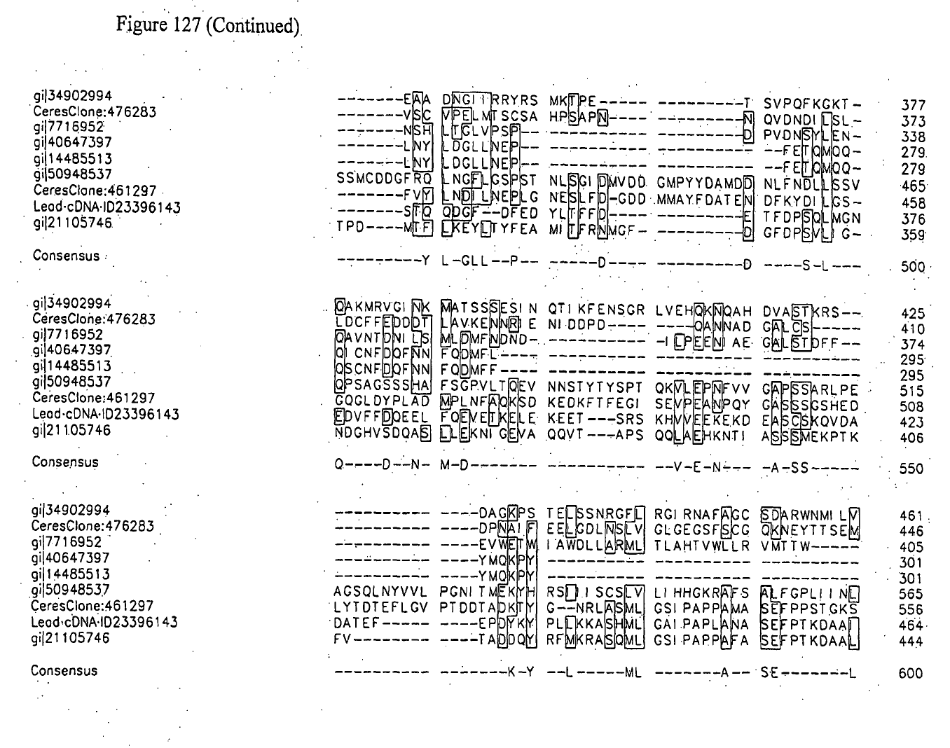

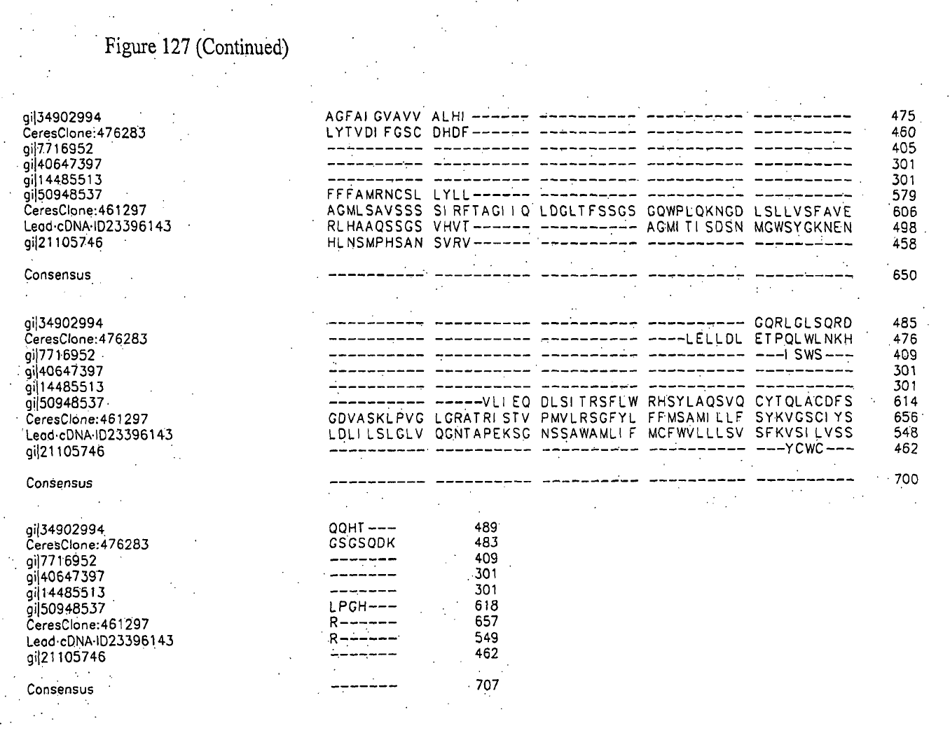

Amino acid sequences of homologs and/or orthologs of the polypeptide having the amino acid sequence set forth in SEQ ID NO:419, SEQ ID NO:579, and SEQ ID NO:1310 are provided in FIG. 39, FIG. 53, and FIG. 127, respectively. Each of FIG. 39, FIG. 53, and FIG. 127 also includes a consensus amino acid sequence determined by aligning homologous and/or orthologous amino acid sequences with the amino acid sequence set forth in SEQ ID NO:419, SEQ ID NO:579, or SEQ ID NO:1310, respectively.

For example, the alignment in FIG. 39 provides the amino acid sequences of cDNA ID 23382112 (SEQ ID NO:419), gi|15293163 (SEQ ID NO:420), gi|34902154 (SEQ ID NO:421), CeresClone:363807 (SEQ ID NO:422), gi|62546183 (SEQ ID NO:423), gi|15148914 (SEQ ID NO:424), gi|56744294 (SEQ ID NO:425), gi|56785066 (SEQ ID NO:428), gi|51702424 (SEQ ID NO:429), gi|52353038 (SEQ ID NO:430), gi|21105748 (SEQ ID NO:431), and gi|4218535 (SEQ ID NO:432). Other homologs and/or orthologs of SEQ ID NO:419 include Public GI no. 51871853 (SEQ ID NO:426) and Public GI no. 53749460 (SEQ ID NO:427).

The alignment in FIG. 53 provides the amino acid sequences of cDNA ID 23467847 (5109D1; SEQ ID NO:579), gi|63252923 (SEQ ID NO:580), CeresClone:363807 (SEQ ID NO:581), gi|58013003 (SEQ ID NO:582), gi|52353038 (SEQ ID NO:583), gi|34902154 (SEQ ID NO:584), gi|21105748 (SEQ ID NO:585), gi|66275772 (SEQ ID NO:586), gi|53749460 (SEQ ID NO:587), and gi|15148914 (SEQ ID NO:588).

The alignment in FIG. 127 provides the amino acid sequences of cDNA ID 23396143 (SEQ ID NO:1310), gi|50948537 (SEQ ID NO:1312), CeresClone:476283 (SEQ ID NO:1313), gi|7716952 (SEQ ID NO:1314), gi|21105746 (SEQ ID NO:1315), gi|40647397 (SEQ ID NO:1316), gi|34902994 (SEQ ID NO:1317), gi|14485513 (SEQ ID NO:1318), and CeresClone:461297 (SEQ ID NO:1319). Other homologs and/or orthologs of SEQ ID NO:1310 include Public GI no. 50948535 (SEQ ID NO:1311).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:420-432, SEQ ID NOs:580-588, SEQ ID NOs:1311-1319, or the consensus sequences set forth in FIG. 39, FIG. 53, or FIG. 127.

A regulatory protein can contain a Pterin_{—}4a domain characteristic of a Pterin 4 alpha carbinolamine dehydratase polypeptide. Pterin 4 alpha carbinolamine dehydratase is also known as DCoH (dimerization cofactor of hepatocyte nuclear factor 1-alpha). DCoH is the dimerization cofactor of hepatocyte nuclear factor 1 (HNF-1) that functions as both a transcriptional coactivator and a pterin dehydratase. X-ray crystallographic studies have shown that the ligand binds at four sites per tetrameric enzyme, with little apparent conformational change in the protein. SEQ ID NO:466 and SEQ ID NO:1202 set forth the amino acid sequence of DNA clones, identified herein as cDNA ID 23370421 (SEQ ID NO:465) and cDNA ID 23785125 (SEQ ID NO:1201), respectively, each of which is predicted to encode a polypeptide containing a Pterin_{—}4a domain.

A regulatory protein can comprise the amino acid sequence set forth in SEQ ID NO:466 or SEQ ID NO:1202. Alternatively, a regulatory protein can be a homolog, ortholog, or variant of the polypeptide having the amino acid sequence set forth in SEQ ID NO:466 or SEQ ID NO:1202. For example, a regulatory protein can have an amino acid sequence with at least 55% sequence identity, e.g., 55%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:466 or SEQ ID NO:1202.

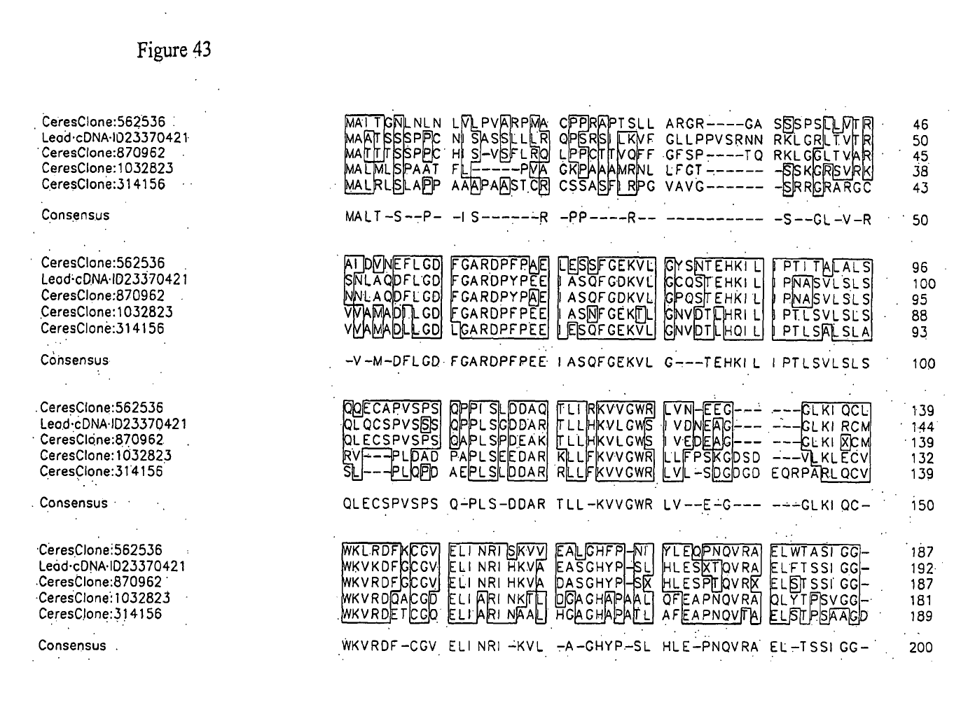

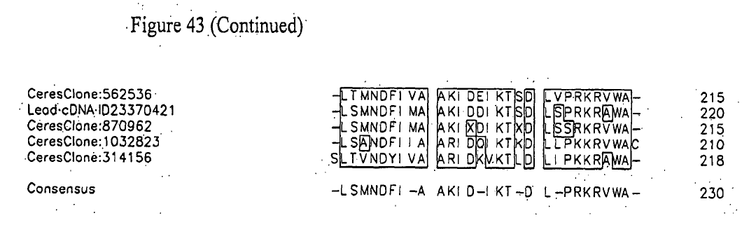

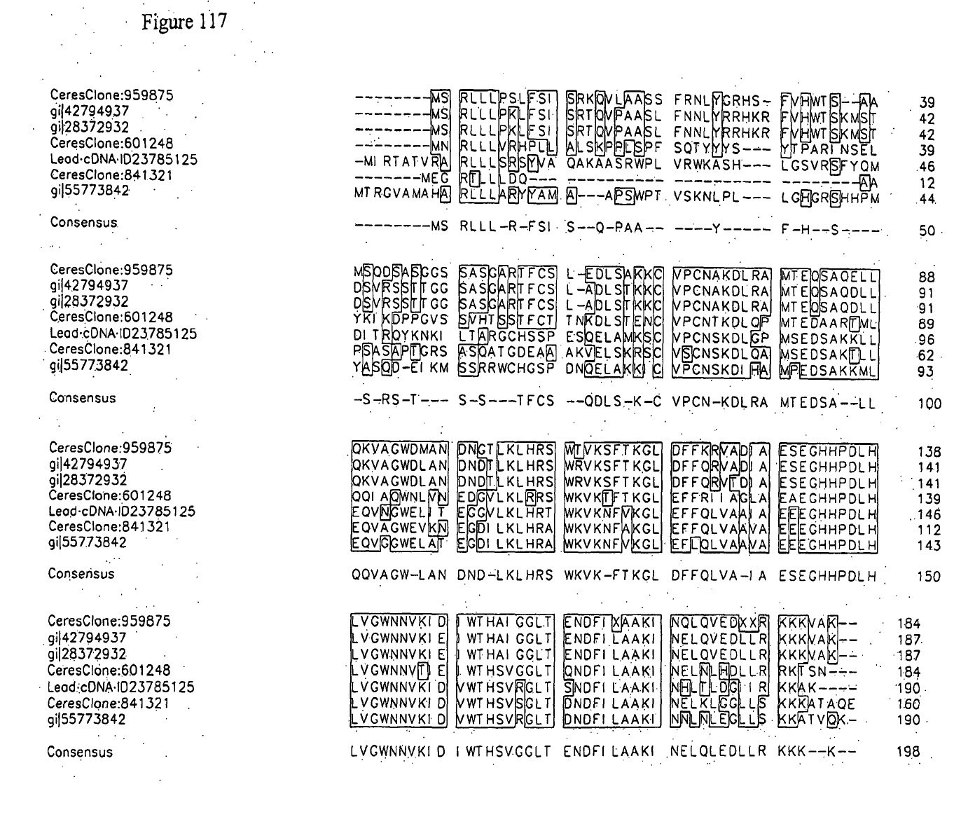

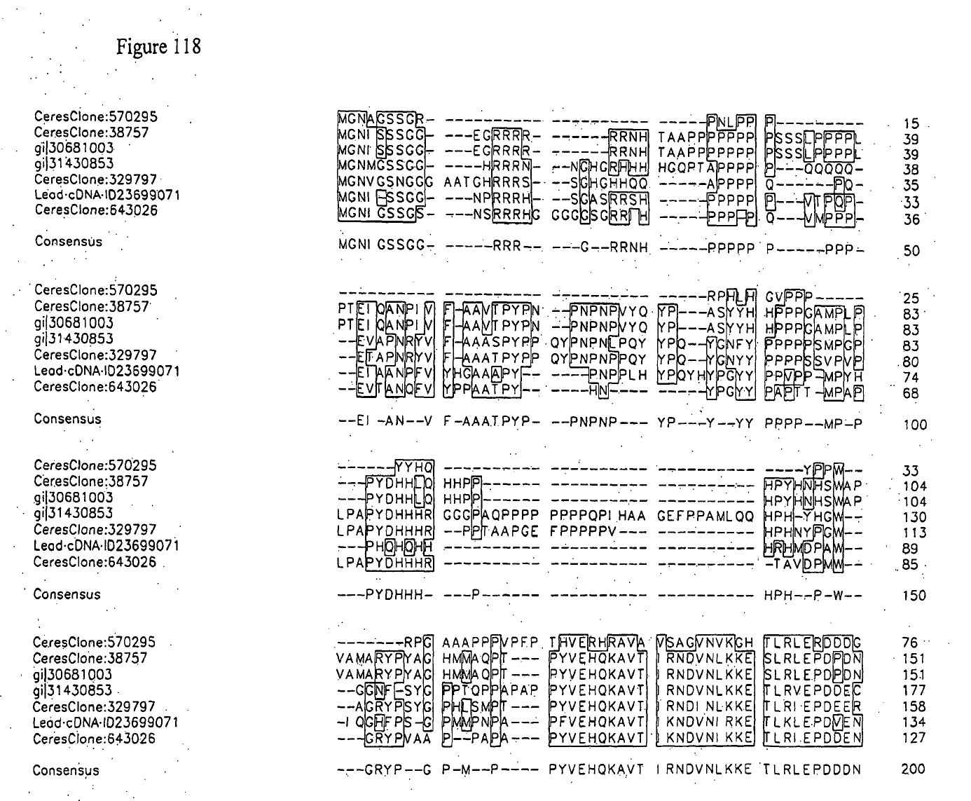

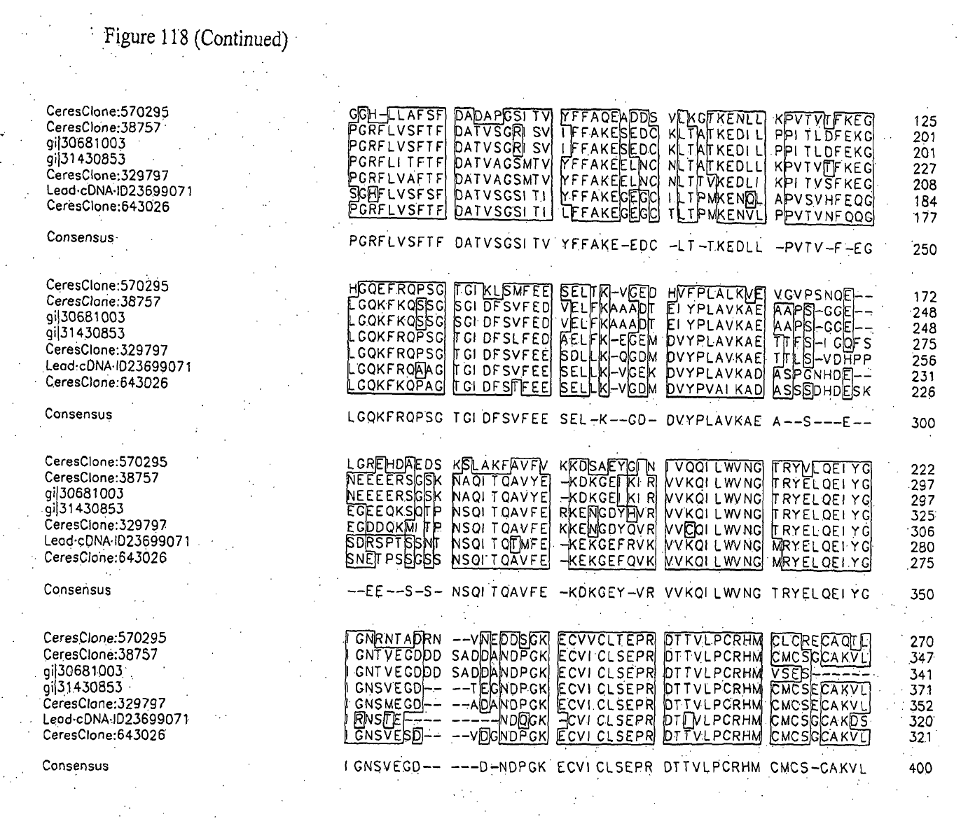

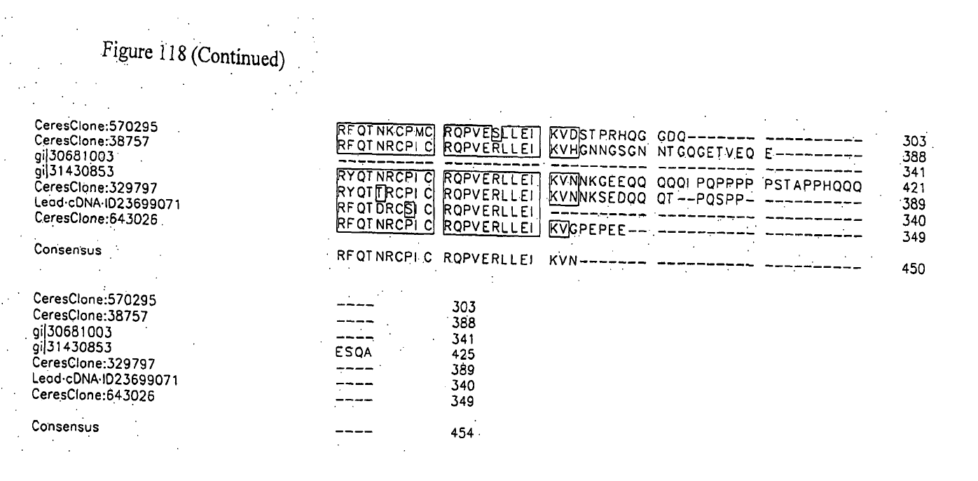

Amino acid sequences of homologs and/or orthologs of the polypeptide having the amino acid sequence set forth in SEQ ID NO:466 and SEQ ID NO:1202 are provided in FIG. 43 and FIG. 117, respectively. Each of FIG. 43 and FIG. 117 also includes a consensus amino acid sequence determined by aligning homologous and/or orthologous amino acid sequences with the amino acid sequence set forth in SEQ ID NO:466 or SEQ ID NO:1202, respectively.

For example, the alignment in FIG. 43 provides the amino acid sequences of cDNA ID 23370421 (SEQ ID NO: 466), CeresClone:870962 (SEQ ID NO:467), CeresClone:562536 (SEQ ID NO:468), CeresClone:1032823 (SEQ ID NO:469), and CeresClone:314156 (SEQ ID NO:470).

The alignment in FIG. 117 provides the amino acid sequences of cDNA ID 23785125 (SEQ ID NO:1202), CeresClone:841321 (SEQ ID NO:1203), gi|55773842 (SEQ ID NO:1204), CeresClone:601248 (SEQ ID NO:1205), gi|42794937 (SEQ ID NO:1206), CeresClone:959875 (SEQ ID NO:1207), and gi|28372932 (SEQ ID NO:1208).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:467-470, SEQ ID NOs:1203-1208, or the consensus sequences set forth in FIG. 43 or FIG. 117.

A regulatory protein can contain a Frigida domain characteristic of a Frigida-like polypeptide. The Frigida-like polypeptide family is composed of plant polypeptides that are similar to the Arabidopsis thaliana FRIGIDA polypeptide. The FRIGIDA polypeptide, which is probably a nuclear polypeptide, is required for the regulation of flowering time in the late-flowering phenotype and is known to increase RNA levels of flowering locus C. Allelic variation at the FRIGIDA locus is a major determinant of natural variation in flowering time. SEQ ID NO:516 sets forth the amino acid sequence of a DNA clone, identified herein as cDNA ID 23539673 (SEQ ID NO:515), that is predicted to encode a Frigida-like polypeptide.

A regulatory protein can comprise the amino acid sequence set forth in SEQ ID NO:516. Alternatively, a regulatory protein can be a homolog, ortholog, or variant of the polypeptide having the amino acid sequence set forth in SEQ ID NO:516. For example, a regulatory protein can have an amino acid sequence with at least 45% sequence identity, e.g., 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:516.

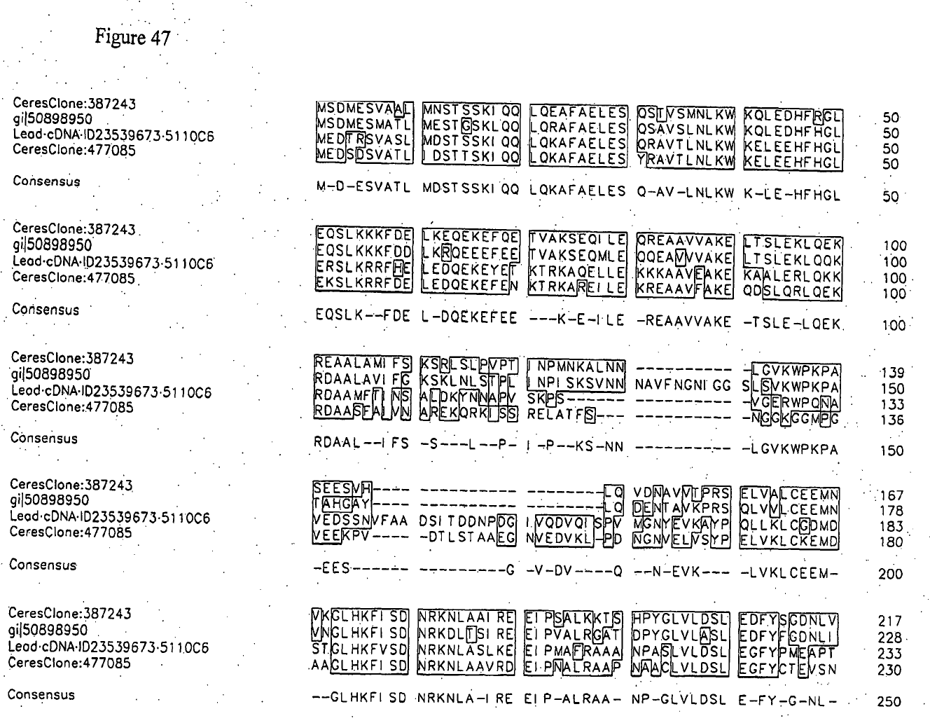

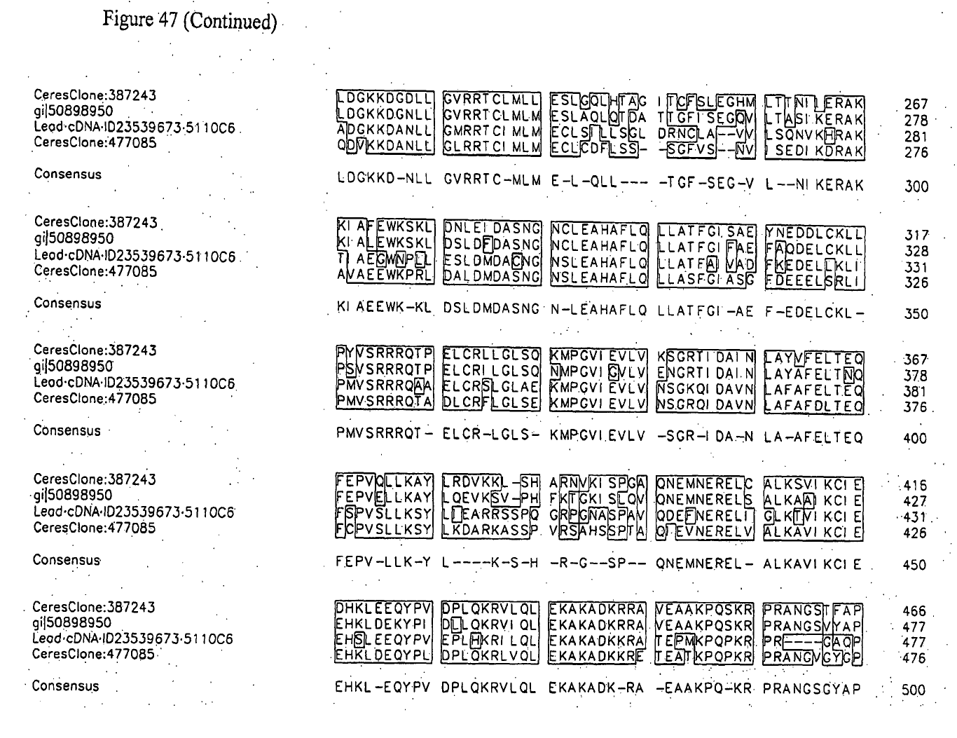

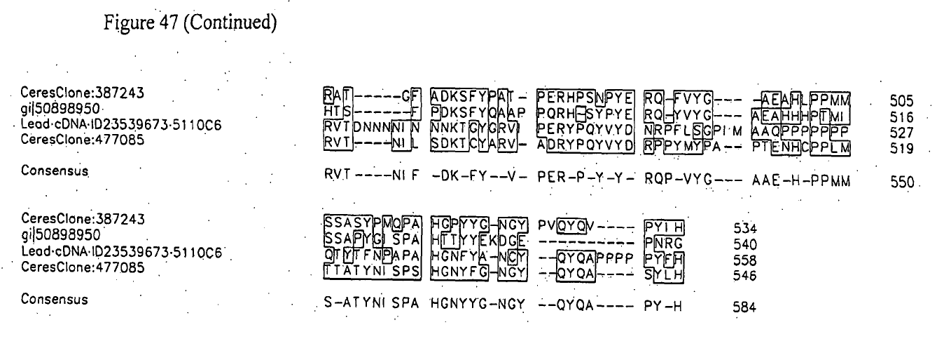

Amino acid sequences of homologs and/or orthologs of the polypeptide having the amino acid sequence set forth in SEQ ID NO:516 are provided in FIG. 47. FIG. 47 also includes a consensus amino acid sequence determined by aligning homologous and/or orthologous amino acid sequences with the amino acid sequence set forth in SEQ ID NO:516.

For example, the alignment in FIG. 47 provides the amino acid sequences of cDNA ID 23539673 (5110C6; SEQ ID NO:516), CeresClone:477085 (SEQ ID NO:517), CeresClone:387243 (SEQ ID NO:518), and gi|50898950 (SEQ ID NO:520). Other homologs and/or orthologs of SEQ ID NO:516 include Ceres CLONE ID no. 379975 (SEQ ID NO:519) and Public GI no. 50898952 (SEQ ID NO:521).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:517-521 or the consensus sequence set forth in FIG. 47.

A regulatory protein can have an mTERF domain. The human mitochondrial transcription termination factor (mTERF) polypeptide possesses three putative leucine zippers, one of which is bipartite. The mTERF polypeptide also contains two widely spaced basic domains. Both of the basic domains and the three leucine zipper motifs are necessary for DNA binding. The mTERF polypeptide binds DNA as a monomer. While evidence of intramolecular leucine zipper interactions exists, the leucine zippers are not implicated in dimerization, unlike other leucine zippers. The rest of the mTERF family consists of hypothetical proteins. SEQ ID NO:574, SEQ ID NO:701, and SEQ ID NO:1378 set forth the amino acid sequences of DNA clones, identified herein as cDNA ID 23653450 (SEQ ID NO:573), cDNA ID 23512013 (SEQ ID NO:700), and cDNA ID 23368763 (SEQ ID NO:1377), respectively, each of which is predicted to encode a polypeptide having an mTERF domain.

A regulatory protein can comprise the amino acid sequence set forth in SEQ ID NO:574, SEQ ID NO:701, or SEQ ID NO:1378. Alternatively, a regulatory protein can be a homolog, ortholog, or variant of the polypeptide having the amino acid sequence set forth in SEQ ID NO:574, SEQ ID NO:701, or SEQ ID NO:1378. For example, a regulatory protein can have an amino acid sequence with at least 50% sequence identity, e.g., 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:574, SEQ ID NO:701, or SEQ ID NO:1378.

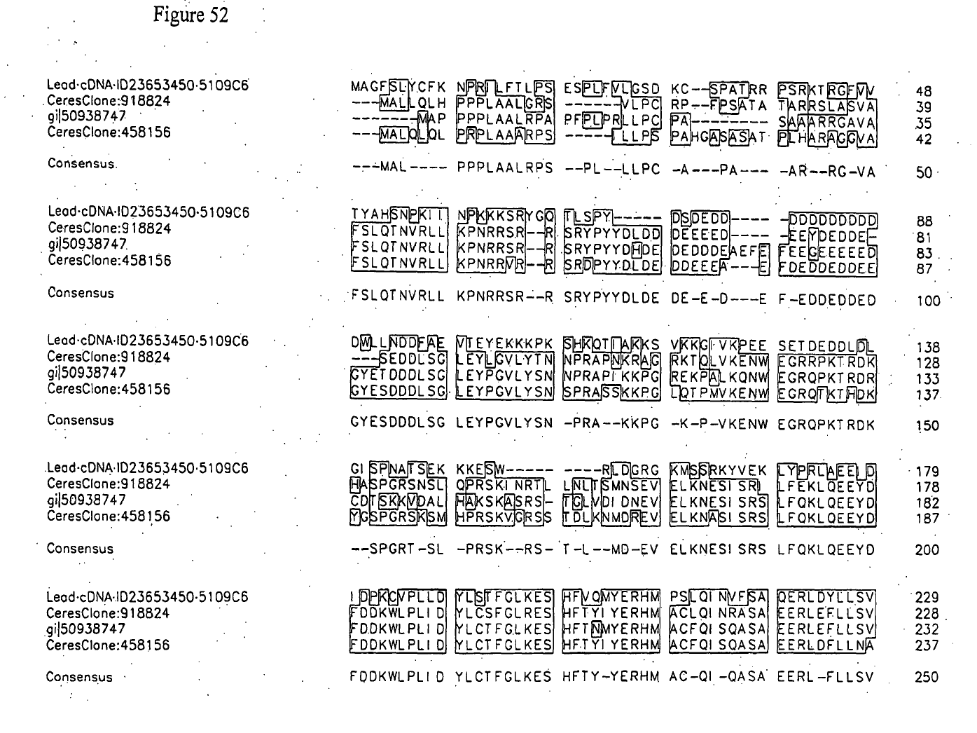

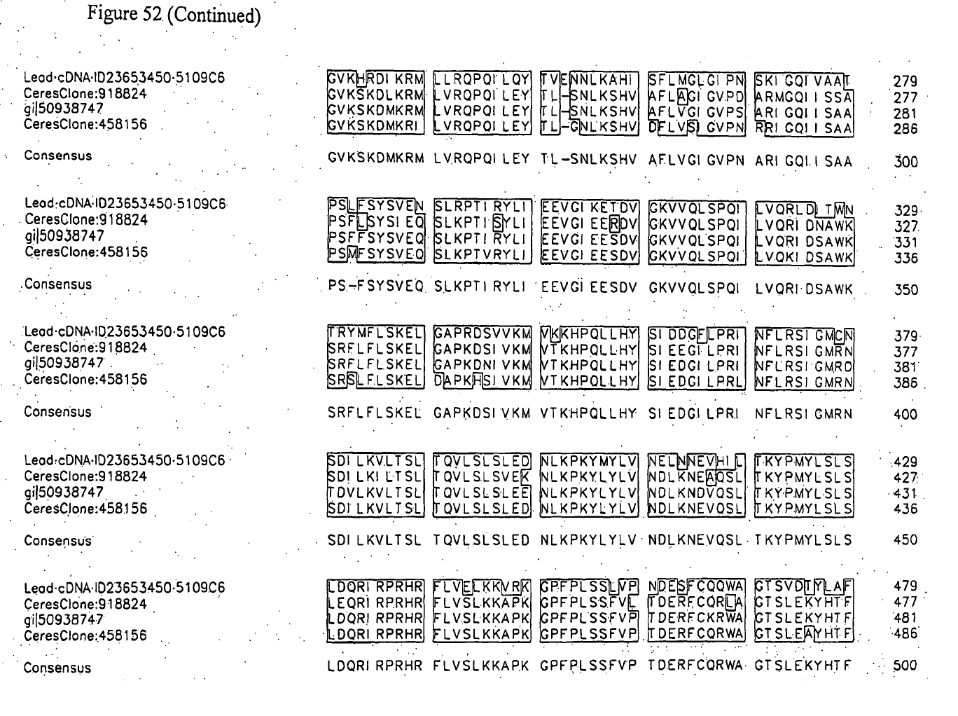

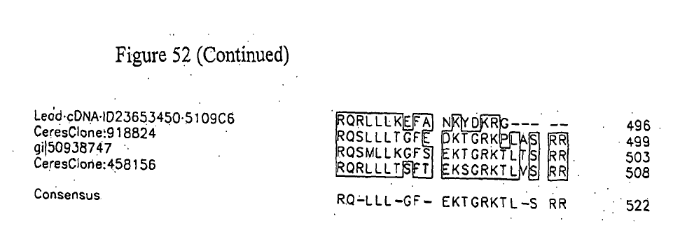

Amino acid sequences of homologs and/or orthologs of the polypeptide having the amino acid sequence set forth in SEQ ID NO:574 are provided in FIG. 52. FIG. 52 also includes a consensus amino acid sequence determined by aligning homologous and/or orthologous amino acid sequences with the amino acid sequence set forth in SEQ ID NO:574.

For example, the alignment in FIG. 52 provides the amino acid sequences of cDNA ID 23653450 (5109C6; SEQ ID NO:574), gi|50938747 (SEQ ID NO:575), CeresClone:458156 (SEQ ID NO:576), and CeresClone:918824 (SEQ ID NO:577).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:575-577, or the consensus sequence set forth in FIG. 52.

A regulatory protein can contain a SAP domain, a WGR domain, a Poly(ADP-ribose) polymerase catalytic domain (PARP), and a Poly(ADP-ribose) polymerase regulatory domain (PARP_reg). The SAP motif, named after SAF-A/B, Acinus and PIAS, is a putative DNA binding domain found in diverse nuclear proteins involved in chromosomal organization. The WGR domain, which is between 70 and 80 residues in length, is found in a variety of polyA polymerases as well as the E. coli molybdate metabolism regulator P33345 and other proteins of unknown function. The domain is named after the most conserved central motif, WGR, and may be a nucleic acid binding domain. Poly(ADP-ribose) polymerase catalyses the covalent attachment of ADP-ribose units from NAD+ to itself and to a limited number of other DNA binding proteins, which decreases their affinity for DNA. Poly(ADP-ribose) polymerase is a regulatory component induced by DNA damage and is involved in the regulation of various cellular processes such as differentiation, proliferation, and regulation of the molecular events involved in the recovery of the cell from DNA damage. The carboxyl-terminal region is the most highly conserved region of the protein. The C-terminal catalytic domain of the polymerase is almost always associated with the N-terminal regulatory domain. The regulatory domain consists of a duplication of two helix-loop-helix structural repeats. SEQ ID NO:211 sets forth the amino acid sequence of a DNA clone, identified herein as cDNA ID 12676498 (SEQ ID NO:210), that is predicted to encode a polypeptide containing a SAP domain, a WGR domain, a PARP domain, and a PARP_reg domain.

A regulatory protein can comprise the amino acid sequence set forth in SEQ ID NO:211. Alternatively, a regulatory protein can be a homolog, ortholog, or variant of the polypeptide having the amino acid sequence set forth in SEQ ID NO:211. For example, a regulatory protein can have an amino acid sequence with at least 55% sequence identity, e.g., 55%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:211.

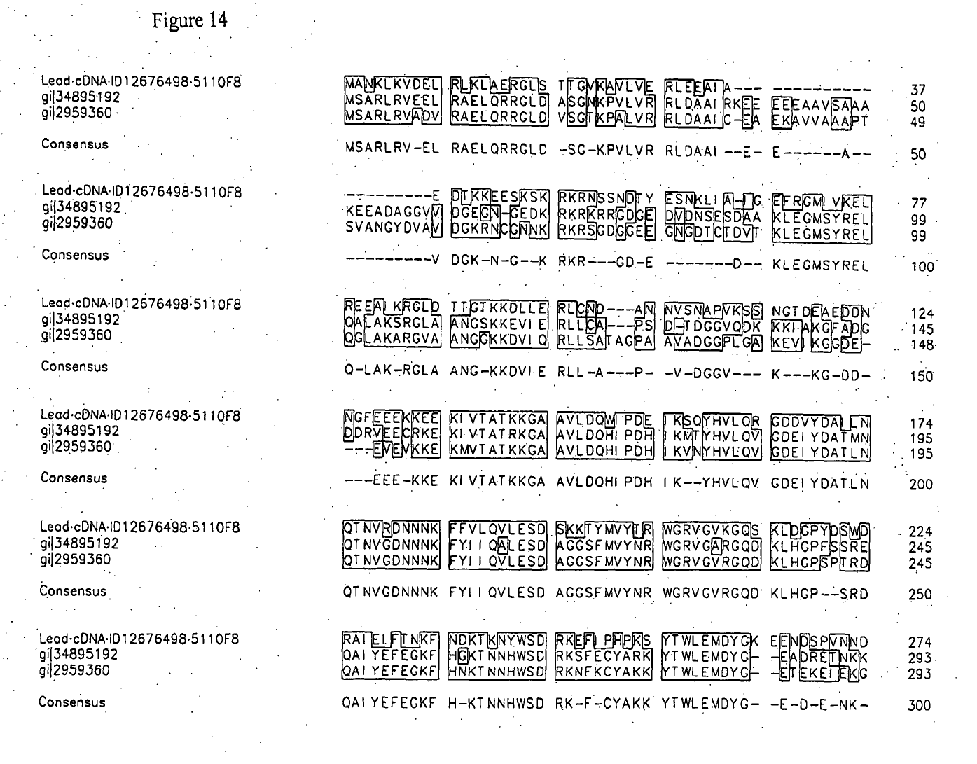

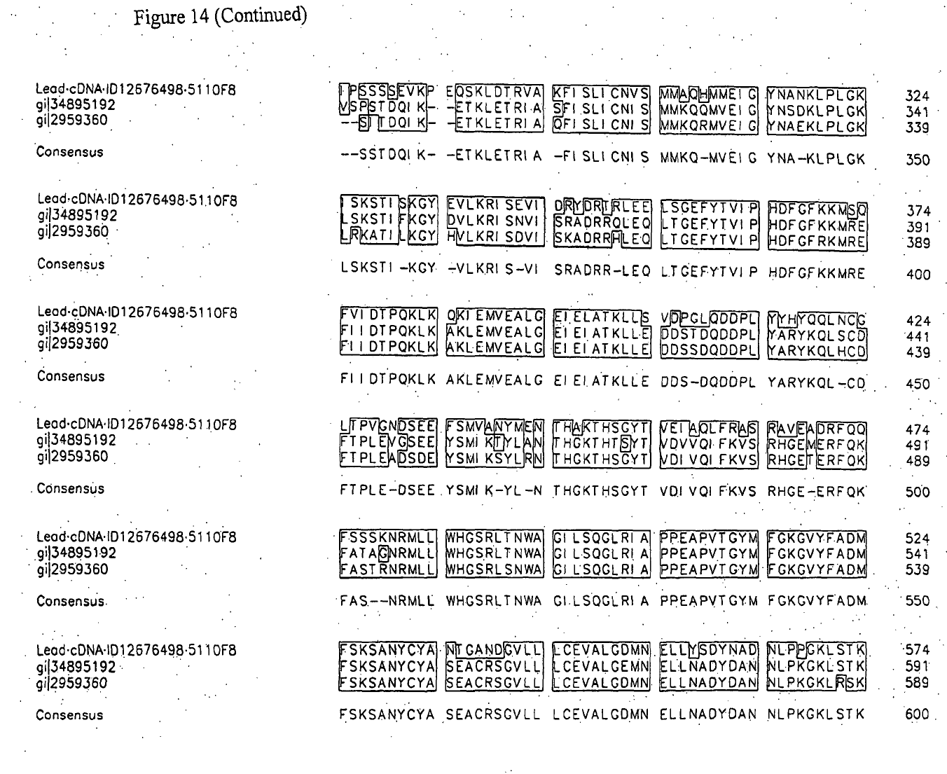

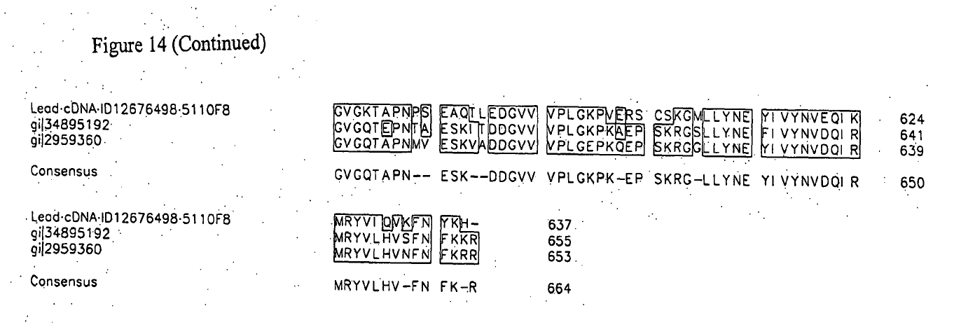

Amino acid sequences of homologs and/or orthologs of the polypeptide having the amino acid sequence set forth in SEQ ID NO:211 are provided in FIG. 14. FIG. 14 also includes a consensus amino acid sequence determined by aligning homologous and/or orthologous amino acid sequences with the amino acid sequence set forth in SEQ ID NO:211.

For example, the alignment in FIG. 14 provides the amino acid sequences of cDNA ID 12676498 (5110F8; SEQ ID NO:211), gi|34895192 (SEQ ID NO:212) and gi|2959360 (SEQ ID NO:213). Other homologs and/or orthologs of SEQ ID NO:211 include Public GI no. 53792821 (SEQ ID NO:214).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:212-214 or the consensus sequence set forth in FIG. 14.

A regulatory protein can contain a Histone domain characteristic of a core histone H2A/H2B/H3/H4 polypeptide. The core histones, together with other DNA binding proteins, form a superfamily defined by a common fold and distant sequence similarities. Some proteins contain local homology domains related to the histone fold. SEQ ID NO:1138 sets forth the amino acid sequence of a DNA clone, identified herein as cDNA ID 23383311 (SEQ ID NO:1137), that is predicted to encode a polypeptide containing a Histone domain.

A regulatory protein can comprise the amino acid sequence set forth in SEQ ID NO:1138. Alternatively, a regulatory protein can be a homolog, ortholog, or variant of the polypeptide having the amino acid sequence set forth in SEQ ID NO:1138. For example, a regulatory protein can have an amino acid sequence with at least 60% sequence identity, e.g., 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:1138.

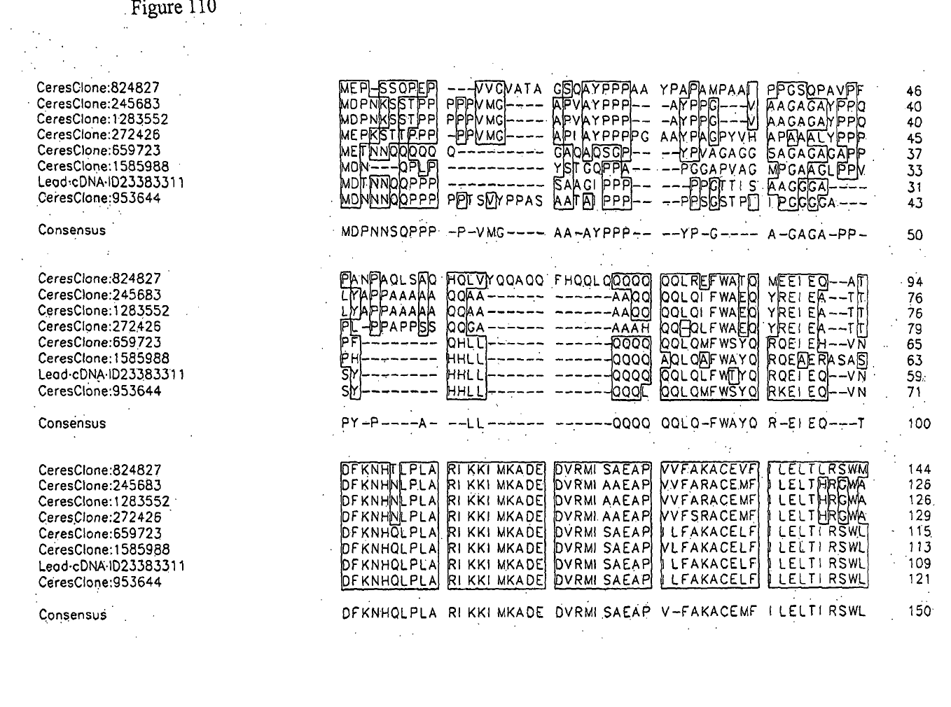

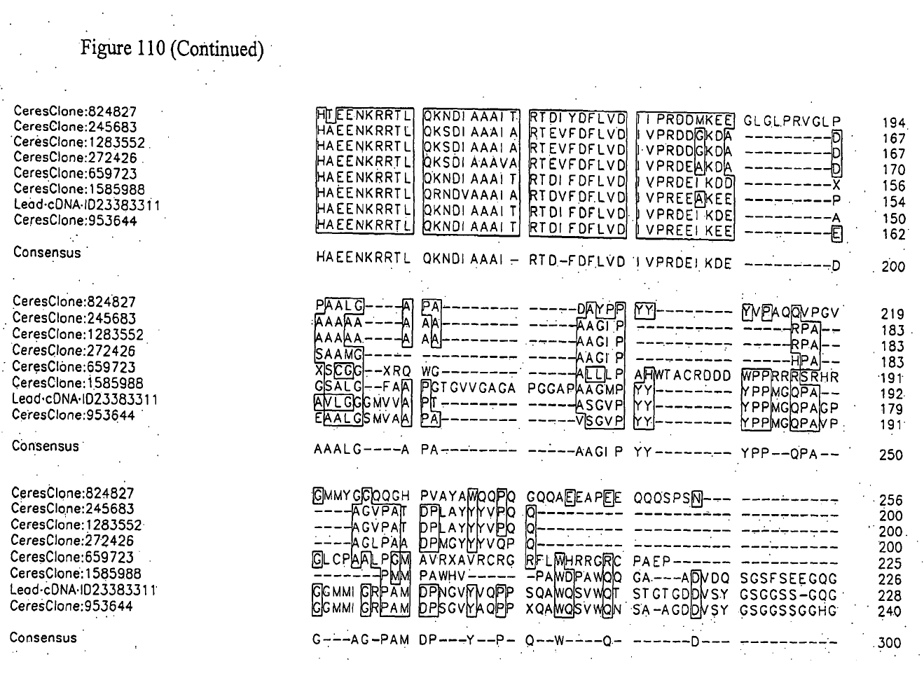

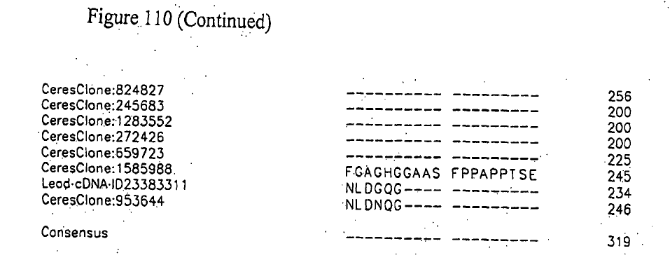

Amino acid sequences of homologs and/or orthologs of the polypeptide having the amino acid sequence set forth in SEQ ID NO:1138 are provided in FIG. 110. FIG. 110 also includes a consensus amino acid sequence determined by aligning homologous and/or orthologous amino acid sequences with the amino acid sequence set forth in SEQ ID NO:1138.

For example, the alignment in FIG. 110 provides the amino acid sequences of cDNA ID 23383311 (SEQ ID NO:1138), CeresClone:659723 (SEQ ID NO:1139), CeresClone:953644 (SEQ ID NO:1140), CeresClone:1585988 (SEQ ID NO:1141), CeresClone:245683 (SEQ ID NO:1142), CeresClone:1283552 (SEQ ID NO:1143), CeresClone:272426 (SEQ ID NO:1144), and CeresClone:824827 (SEQ ID NO:1145).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:1139-1145 or the consensus sequence set forth in FIG. 110.

A regulatory protein can contain an XS zinc finger domain, which is a putative nucleic acid binding zinc finger found in proteins that also contain an XS domain and an XH domain. The XH (rice gene X Homology) domain is found in a family of plant proteins including Oryza saliva Putative X1. The XH domain is between 124 and 145 residues in length and contains a conserved glutamate residue that may be functionally important. The XS (rice gene X and SGS3) domain is found in a family of plant proteins including gene X and SGS3. SGS3 is thought to be involved in post-transcriptional gene silencing (PTGS). The XS domain contains a conserved aspartate residue that may be functionally important. XS domain-containing proteins contain coiled-coils, which suggests that they oligomerize. Most coiled-coil proteins form either a dimeric or a trimeric structure. It is possible that different members of the XS domain family oligomerize via their coiled-coils to form a variety of complexes. The XS and XH domains may interact since they are often fused. SEQ ID NO:652 sets forth the amino acid sequence of a DNA clone, identified herein as cDNA ID 23502669 (SEQ ID NO:651), that is predicted to encode a polypeptide containing an XS zinc finger domain, an XS domain, and an XH domain.

A regulatory protein can comprise the amino acid sequence set forth in SEQ ID NO:652. Alternatively, a regulatory protein can be a homolog, ortholog, or variant of the polypeptide having the amino acid sequence set forth in SEQ ID NO:652. For example, a regulatory protein can have an amino acid sequence with at least 35% sequence identity, e.g., 35%, 40%, 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:652.

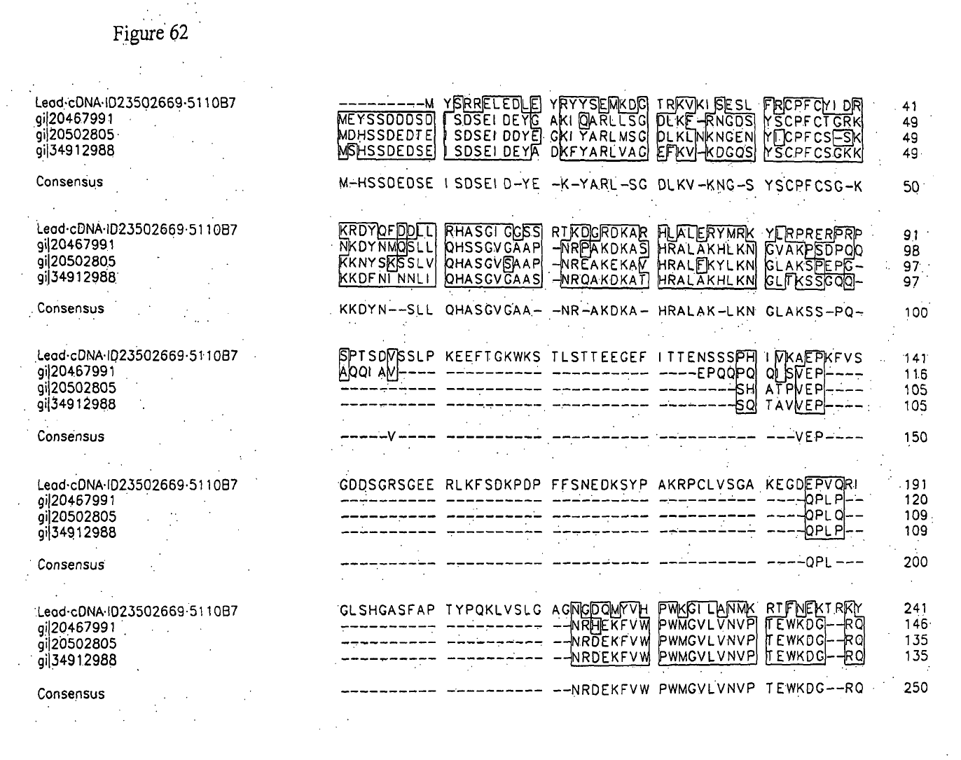

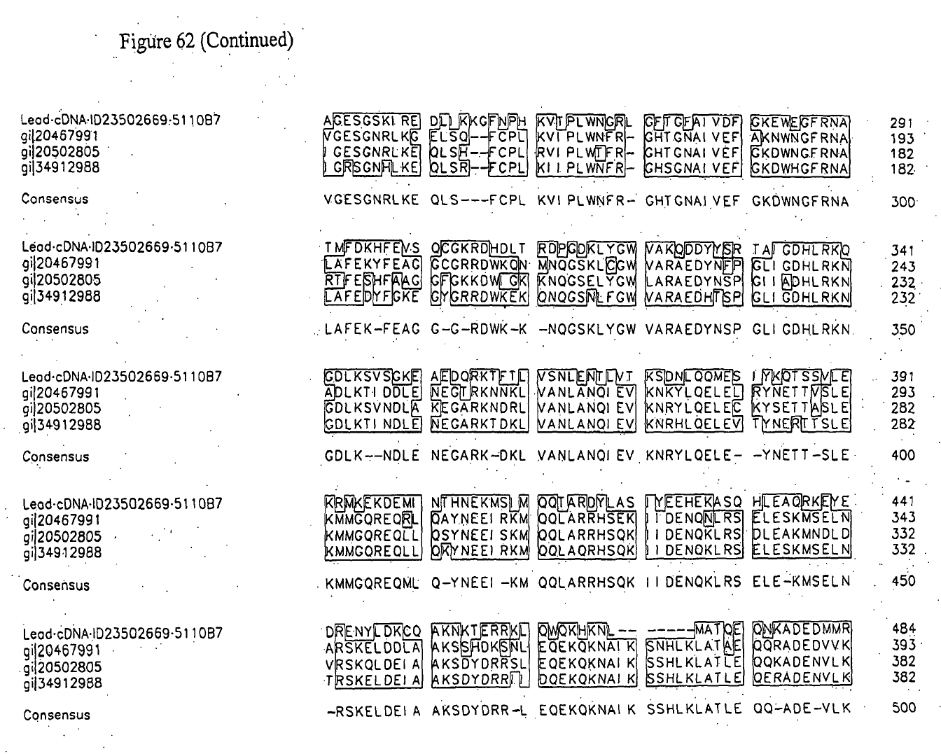

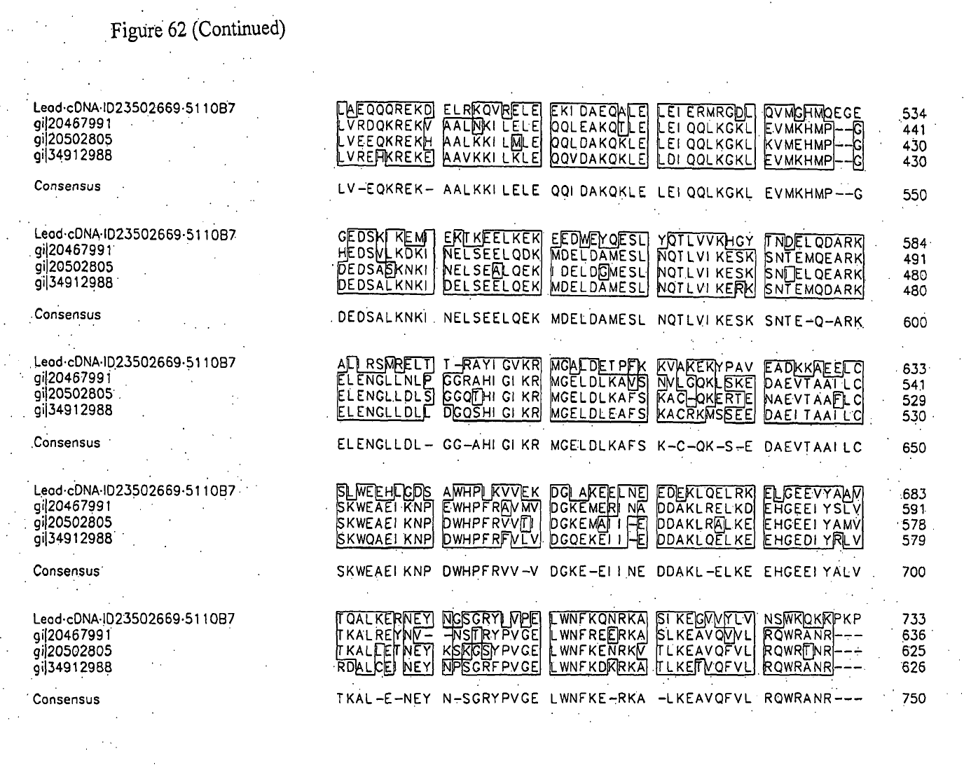

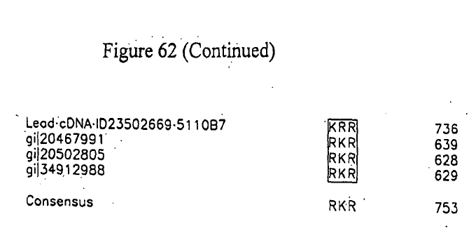

Amino acid sequences of homologs and/or orthologs of the polypeptide having the amino acid sequence set forth in SEQ ID NO:652 are provided in FIG. 62. FIG. 62 also includes a consensus amino acid sequence determined by aligning homologous and/or orthologous amino acid sequences with the amino acid sequence set forth in SEQ ID NO:652.

For example, the alignment in FIG. 62 provides the amino acid sequences of cDNA ID 23502669 (5110B7; SEQ ID NO:652), gi|20502805 (SEQ ID NO:653), gi|34912988 (SEQ ID NO:654), and gi|20467991 (SEQ ID NO:655).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to SEQ ID NOs:653-655 or the consensus sequence set forth in FIG. 62.

A regulatory protein can contain an Acetyltransf_{—}1 domain and an NMT_C domain. The Acetyltransf_{—}1 domain is characteristic of polypeptides belonging to the acetyltransferase (GNAT) family. The GNAT family includes Gcn5-related acetyltransferases, which catalyze the transfer of an acetyl group from acetyl-CoA to the lysine E-amino groups on the N-terminal tails of histones. Many GNATs share several functional domains, including an N-terminal region of variable length, an acetyltransferase domain encompassing conserved sequence motifs, a region that interacts with the coactivator Ada2, and a C-terminal bromodomain that is believed to interact with acetyl-lysine residues. Members of the GNAT family are important for the regulation of cell growth and development. The importance of GNATs is probably related to their role in transcription and DNA repair. The NMT_C domain is present in myristoyl-CoA:protein N-myristoyltransferase (Nmt), which is the enzyme responsible for transferring a myristate group to the N-terminal glycine of a number of cellular eukaryotic and viral proteins. The N and C-terminal domains of NMT are structurally similar, each adopting an acyl-CoA N-acyltransferase-like fold. SEQ ID NO:333 sets forth the amino acid sequence of a DNA clone, identified herein as cDNA ID 23764087 (SEQ ID NO:332), that is predicted to encode a polypeptide containing an Acetyltransf_{—}1 domain and an NMT_C domain.

A regulatory protein can comprise the amino acid sequence set forth in SEQ ID NO:333. Alternatively, a regulatory protein can be a homolog, ortholog, or variant of the polypeptide having the amino acid sequence set forth in SEQ ID NO:333. For example, a regulatory protein can have an amino acid sequence with at least 50% sequence identity, e.g., 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:333.

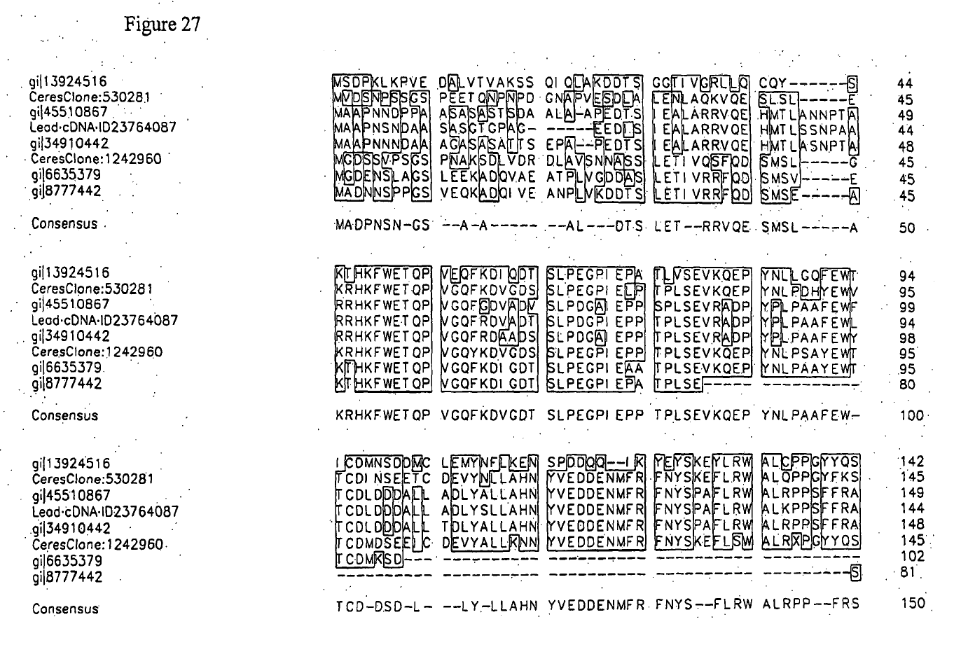

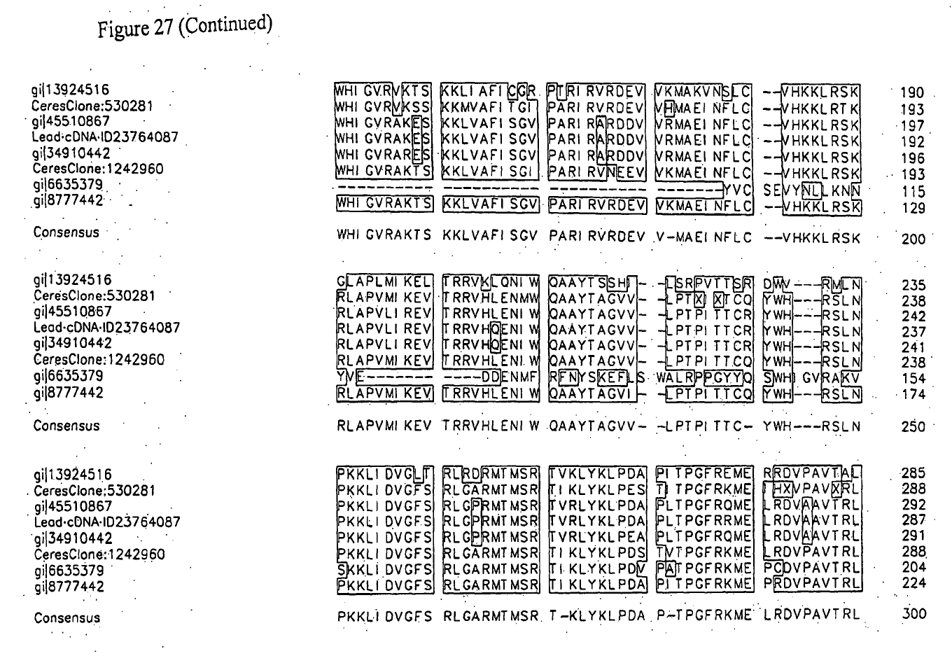

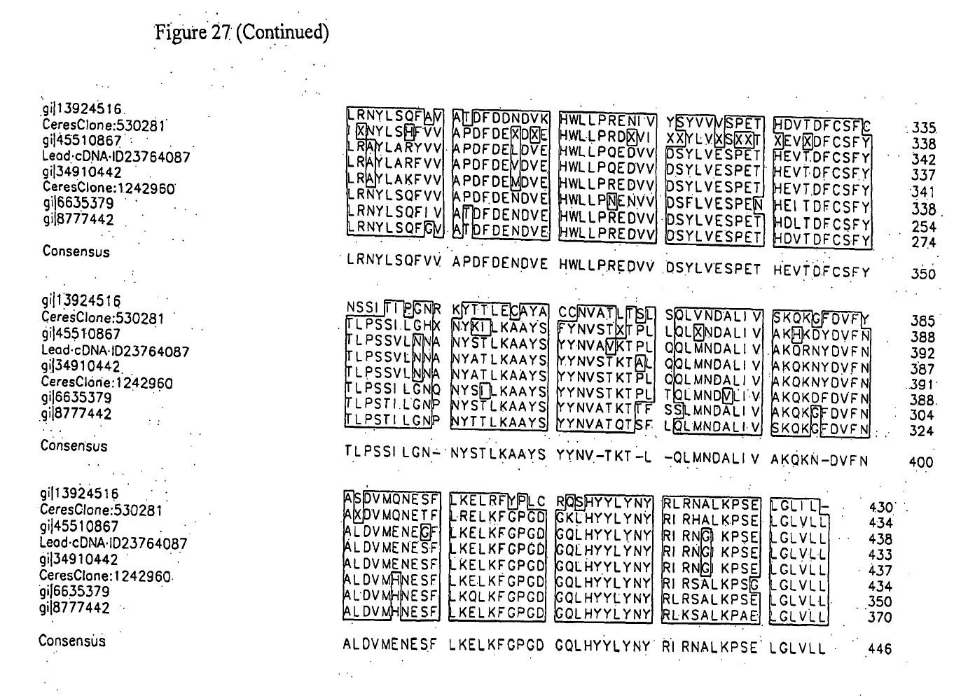

Amino acid sequences of homologs and/or orthologs of the polypeptide having the amino acid sequence set forth in SEQ ID NO:333 are provided in FIG. 27. FIG. 27 also includes a consensus amino acid sequence determined by aligning homologous and/or orthologous amino acid sequences with the amino acid sequence set forth in SEQ ID NO:333.

For example, the alignment in FIG. 27 provides the amino acid sequences of cDNA ID 23764087 (SEQ ID NO:333), gi|34910442 (SEQ ID NO:334), gi|45510867 (SEQ ID NO:335), gi|8777442 (SEQ ID NO:336), CeresClone:1242960 (SEQ ID NO:339), gi|6635379 (SEQ ID NO:340), CeresClone:530281 (SEQ ID NO:341), and gi|13924516 (SEQ ID NO:343). Other homologs and/or orthologs of SEQ ID NO:333 include Ceres CLONE ID no. 36525 (SEQ ID NO:337), Public GI no. 13924514 (SEQ ID NO:338), and Public GI no. 7484992 (SEQ ID NO:342).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:334-343 or the consensus sequence set forth in FIG. 27.

A regulatory protein can contain an AUX_IAA domain. The Aux/IAA family of genes are key regulators of auxin-modified gene expression. The plant hormone auxin (indole-3-acetic acid, IAA) regulates diverse cellular and developmental responses in plants. The Aux/IAA proteins act as repressors of auxin-induced gene expression, possibly by modulating the activity of DNA binding auxin response factors (ARFs). Aux/IAA and ARF are thought to interact through C-terminal protein-protein interaction domains found in both Aux/IAA and ARF. Aux/IAA proteins have also been reported to mediate light responses. Some members of the AUX/IAA family are longer, contain an N-terminal DNA binding domain, and may have an early function in the establishment of vascular and body patterns during embryonic and post-embryonic development in some plants. SEQ ID NO:686, SEQ ID NO:834, SEQ ID NO:1058, and SEQ ID NO:1147 set forth the amino acid sequences of DNA clones, identified herein as cDNA ID 23524514 (SEQ ID NO:685), cDNA ID 23516633 (SEQ ID NO:833), cDNA ID 23371818 (SEQ ID NO:1057), and cDNA ID 23384792 (SEQ ID NO:1146), respectively, each of which is predicted to encode a polypeptide containing an AUX_IAA domain.

A regulatory protein can comprise the amino acid sequence set forth in SEQ ID NO:686, SEQ ID NO:834, SEQ ID NO:1058, or SEQ ID NO:1147. Alternatively, a regulatory protein can be a homolog, ortholog, or variant of the polypeptide having the amino acid sequence set forth in SEQ ID NO:686, SEQ ID NO:834, SEQ ID NO:1058, or SEQ ID NO:1147. For example, a regulatory protein can have an amino acid sequence with at least 40% sequence identity, e.g., 40%, 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:686, SEQ ID NO:834, SEQ ID NO:1058, or SEQ ID NO:1147.

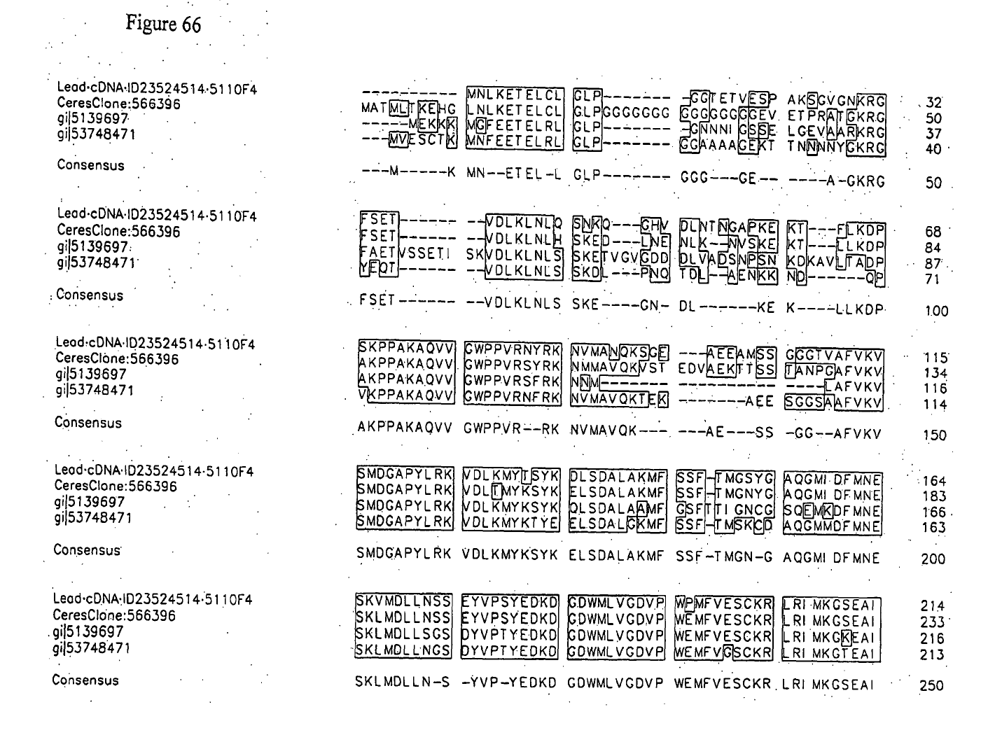

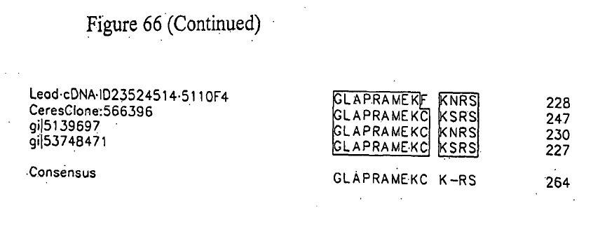

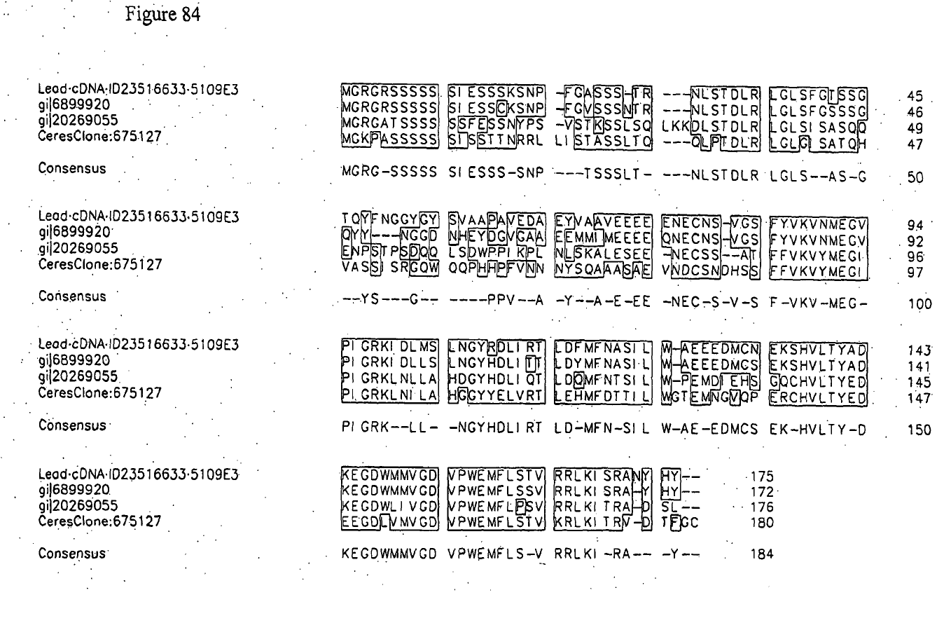

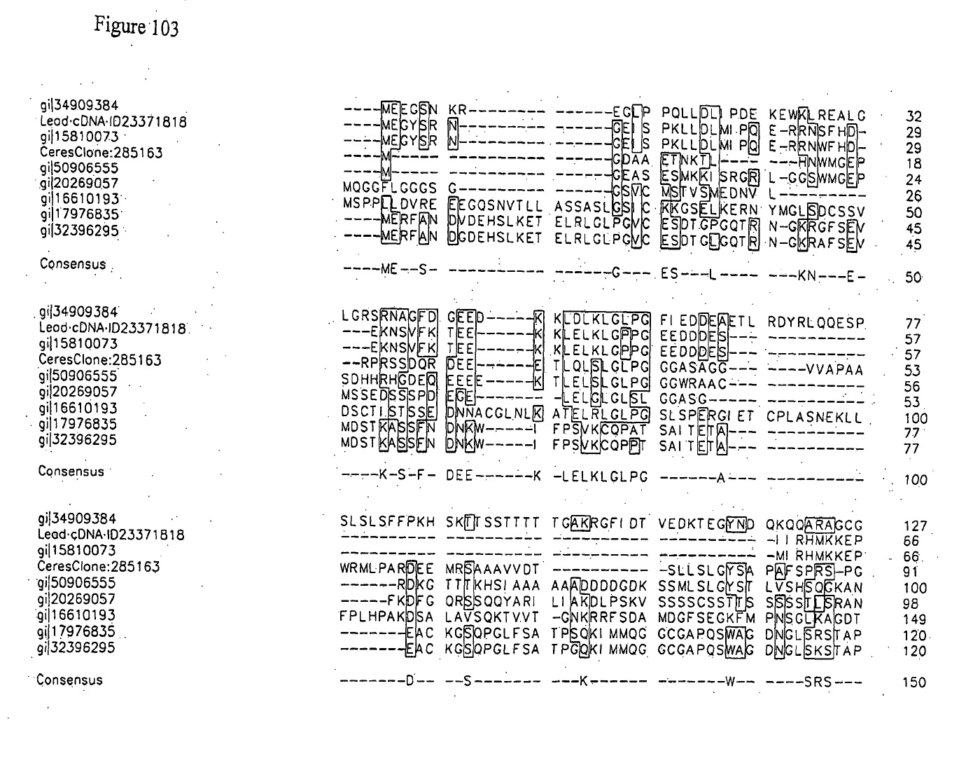

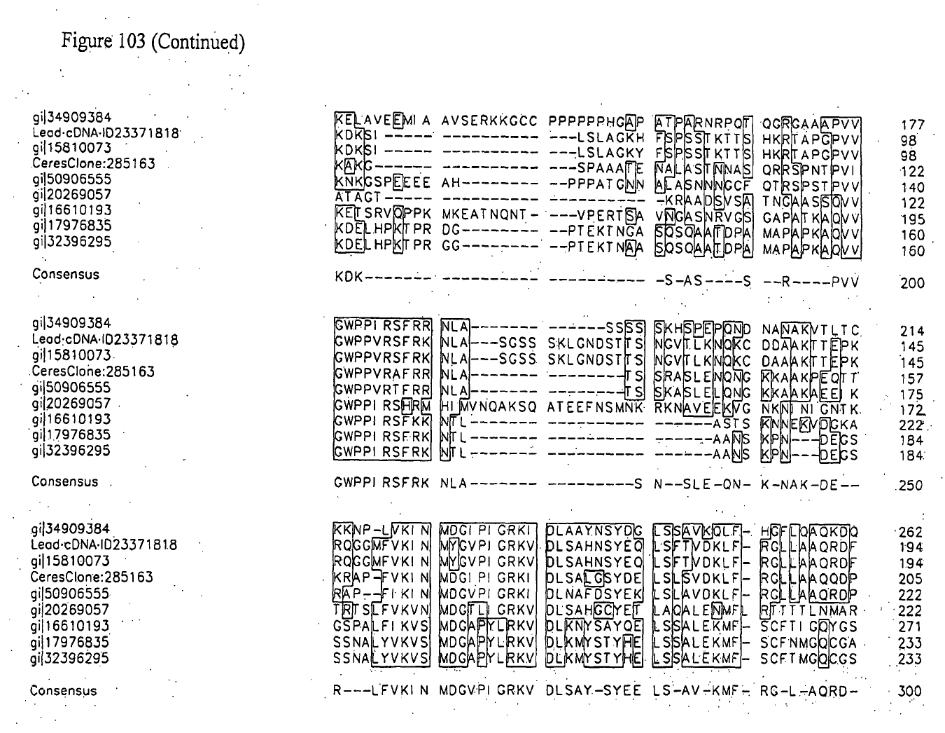

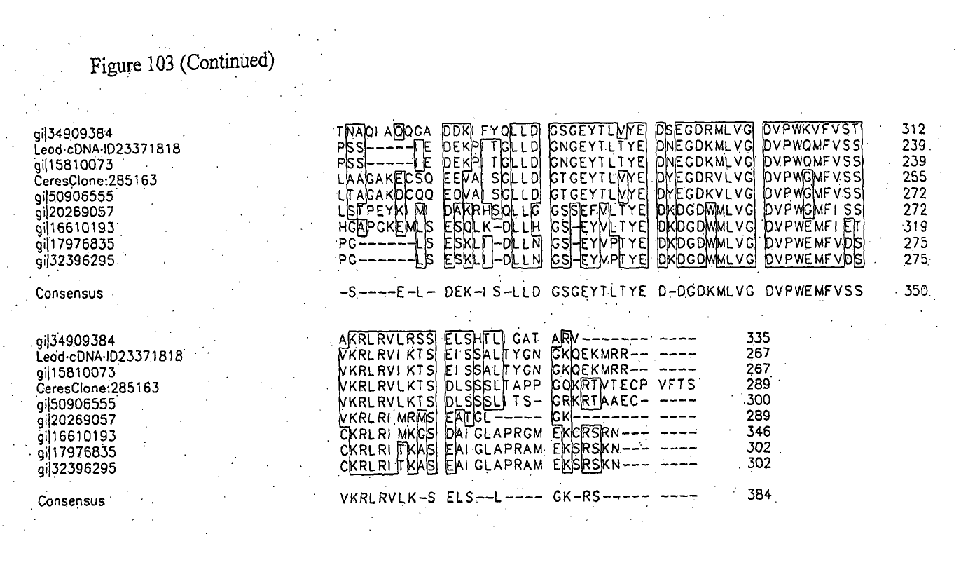

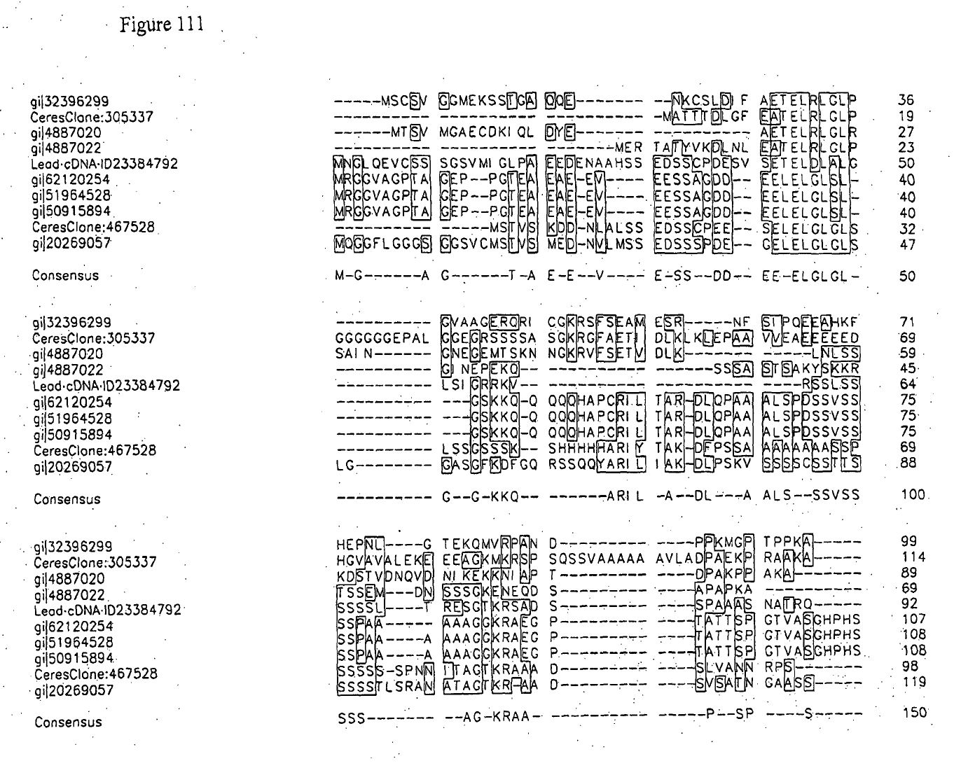

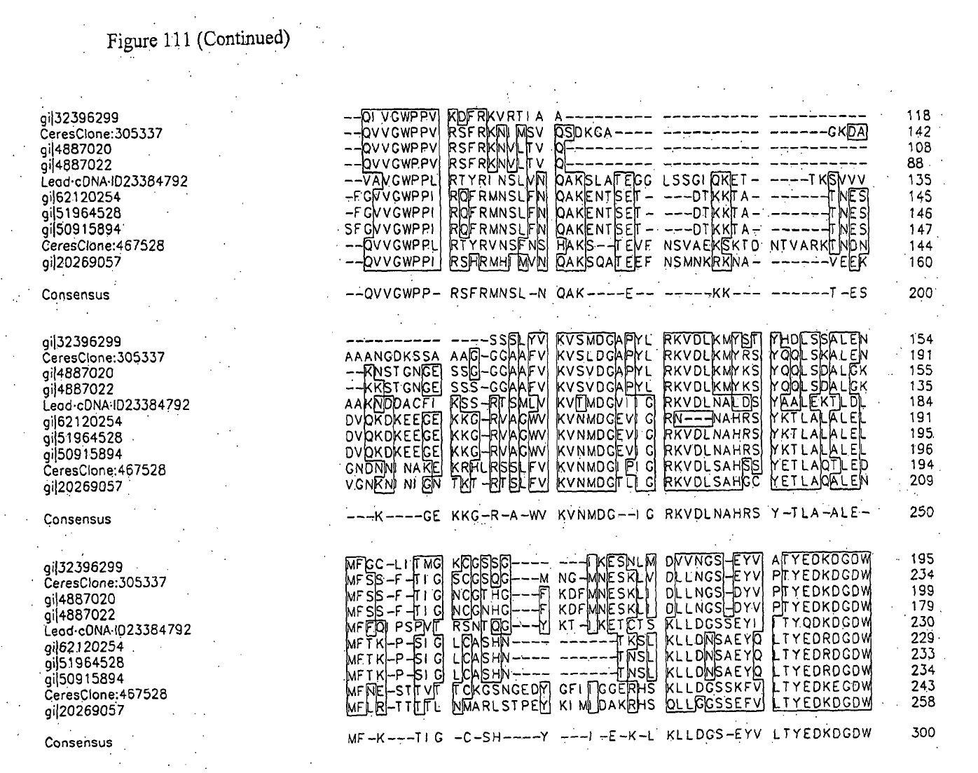

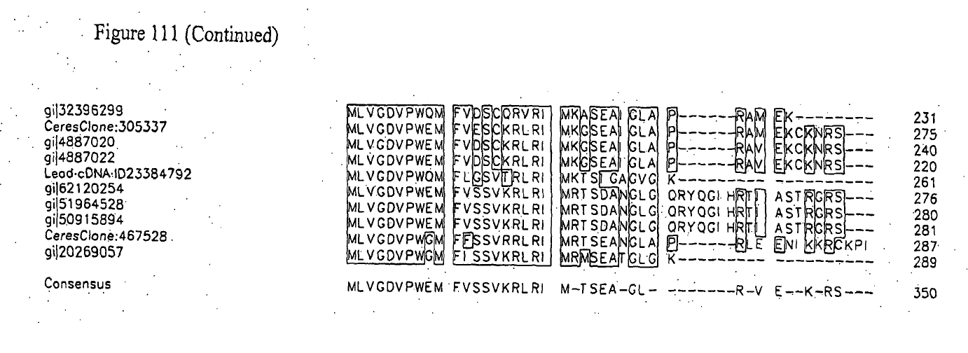

Amino acid sequences of homologs and/or orthologs of the polypeptide having the amino acid sequence set forth in SEQ ID NO:686, SEQ ID NO:834, SEQ ID NO:1058, and SEQ ID NO:1147 are provided in FIG. 66, FIG. 84, FIG. 103, and FIG. 111, respectively. Each of FIG. 66, FIG. 84, FIG. 103, and FIG. 111 also includes a consensus amino acid sequence determined by aligning homologous and/or orthologous amino acid sequences with the amino acid sequence set forth in SEQ ID NO:686, SEQ ID NO:834, SEQ ID NO:1058, or SEQ ID NO:1147, respectively.

For example, the alignment in FIG. 66 provides the amino acid sequences of cDNA ID 23524514 (5110F4; SEQ ID NO:686), CeresClone:566396 (SEQ ID NO:690), gi|5139697 (SEQ ID NO:691), and gi|53748471 (SEQ ID NO:693). Other homologs and/or orthologs of SEQ ID NO:686 include Ceres CLONE ID no. 38286 (SEQ ID NO:687), Public GI no. 21593352 (SEQ ID NO:688), Public GI no. 12083200 (SEQ ID NO:689), and Ceres CLONE ID no. 1113630 (SEQ ID NO:692).

The alignment in FIG. 84 provides the amino acid sequences of cDNA ID 23516633 (5109E3; SEQ ID NO:834), gi|6899920 (SEQ ID NO:835), gi|20269055 (SEQ ID NO:836), and CeresClone:675127 (SEQ ID NO:838). Other homologs and/or orthologs of SEQ ID NO:834 include Public GI no. 20269053 (SEQ ID NO:837).

The alignment in FIG. 103 provides the amino acid sequences of cDNA ID 23371818 (SEQ ID NO:1058), gi|15810073 (SEQ ID NO:1059), CeresClone:285163 (SEQ ID NO:1060), gi|50906555 (SEQ ID NO:1061), gi|34909384 (SEQ ID NO:1062), gi|17976835 (SEQ ID NO:1063), gi|32396295 (SEQ ID NO:1064), gi|16610193 (SEQ ID NO:1065), and gi|20269057 (SEQ ID NO:1066).

The alignment in FIG. 111 provides the amino acid sequences of cDNA ID 23384792 (SEQ ID NO:1147), CeresClone:467528 (SEQ ID NO:1148), gi|20269057 (SEQ ID NO:1149), gi|51964528 (SEQ ID NO:1150), gi|50915894 (SEQ ID NO:1151), gi|32396299 (SEQ ID NO:1152), gi|62120254 (SEQ ID NO:1153), gi|4887020 (SEQ ID NO:1154), gi|4887022 (SEQ ID NO:1155), and CeresClone:305337 (SEQ ID NO:1156).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:687-693, SEQ ID NOs:835-838, SEQ ID NOs:1059-1066, SEQ ID NOs:1148-1156, or the consensus sequences set forth in FIG. 66, FIG. 84, FIG. 103, or FIG. 111.

A regulatory protein can contain one or more tetratricopeptide repeats (TPRs). For example, a regulatory protein can contain a TPR_{—}1 and a TPR_{—}2 motif. Tetratricopeptide repeats, such as TPR_{—}1, TPR_{—}2, TPR_{—}3, and TPR_{—}4, are structural motifs that are present in a wide range of proteins and that mediate protein-protein interactions and assembly of multi-protein complexes. The TPR motif consists of 316 tandem repeats of 34 amino acid residues, although individual TPR motifs can be dispersed in the protein sequence. Sequence alignment of TPR domains has revealed a consensus sequence defined by a pattern of small and large amino acids. TPR motifs have been identified in various different organisms, ranging from bacteria to humans. Proteins containing TPRs are involved in a variety of biological processes, such as cell cycle regulation, transcriptional control, mitochondrial and peroxisomal protein transport, neurogenesis, and protein folding. SEQ ID NO:376 and SEQ ID NO:1158 set forth the amino acid sequences of DNA clones, identified herein as cDNA ID 23662829 (SEQ ID NO:375) and cDNA ID 23360311 (SEQ ID NO:1157), respectively, each of which is predicted to encode a polypeptide containing a TPR_{—}1 and a TPR_{—}2 motif.

In some cases, a regulatory protein can contain a TPR_{—}1 motif, a TPR_{—}2 motif, a TPR_{—}4 motif, and an efhand domain. The EF-hand domain is a type of calcium-binding domain shared by many calcium-binding proteins belong to the same evolutionary family. EF hand domains can be divided into two classes: signaling proteins and buffering/transport proteins. The first group is the largest and includes the most well-known members of the family such as calmodulin, troponin C, and S100B. These proteins typically undergo a calcium-dependent conformational change which opens a target binding site. Members of the buffering/transport protein group, which is represented by calbindin D9k, do not undergo calcium-dependent conformational changes. The EF-hand domain consists of a twelve residue loop flanked on both side by a twelve residue alpha-helical domain. In an EF-hand loop the calcium ion is coordinated in a pentagonal bipyramidal configuration. The six residues involved in the binding are in positions 1, 3, 5, 7, 9 and 12, and these residues are denoted by X, Y, Z, −Y, −X and −Z. The invariant Glu or Asp at position 12 provides two oxygens for liganding Ca (bidentate ligand). SEQ ID NO:671 sets forth the amino acid sequence of a DNA clone, identified herein as cDNA ID 23503971 (SEQ ID NO:670), that is predicted to encode a polypeptide containing a TPR_{—}1 motif, a TPR_{—}2 motif, a TPR_{—}4 motif, and an efhand domain.

A regulatory protein can comprise the amino acid sequence set forth in SEQ ID NO:376, SEQ ID NO:1158, or SEQ ID NO:671. Alternatively, a regulatory protein can be a homolog, ortholog, or variant of the polypeptide having the amino acid sequence set forth in SEQ ID NO:376, SEQ ID NO:1158, or SEQ ID NO:671. For example, a regulatory protein can have an amino acid sequence with at least 50% sequence identity, e.g., 50%, 55%, 60%, 65%, 70%, 75%, 80%, 85%, 90%, 95%, 97%, 98%, or 99% sequence identity, to the amino acid sequence set forth in SEQ ID NO:376, SEQ ID NO:1158, or SEQ ID NO:671.

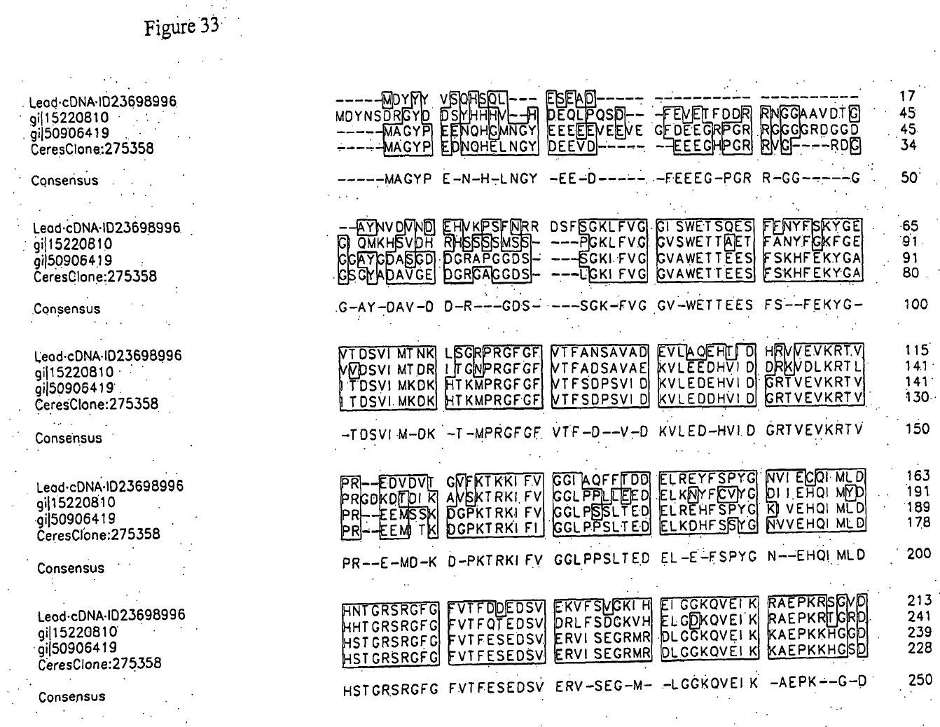

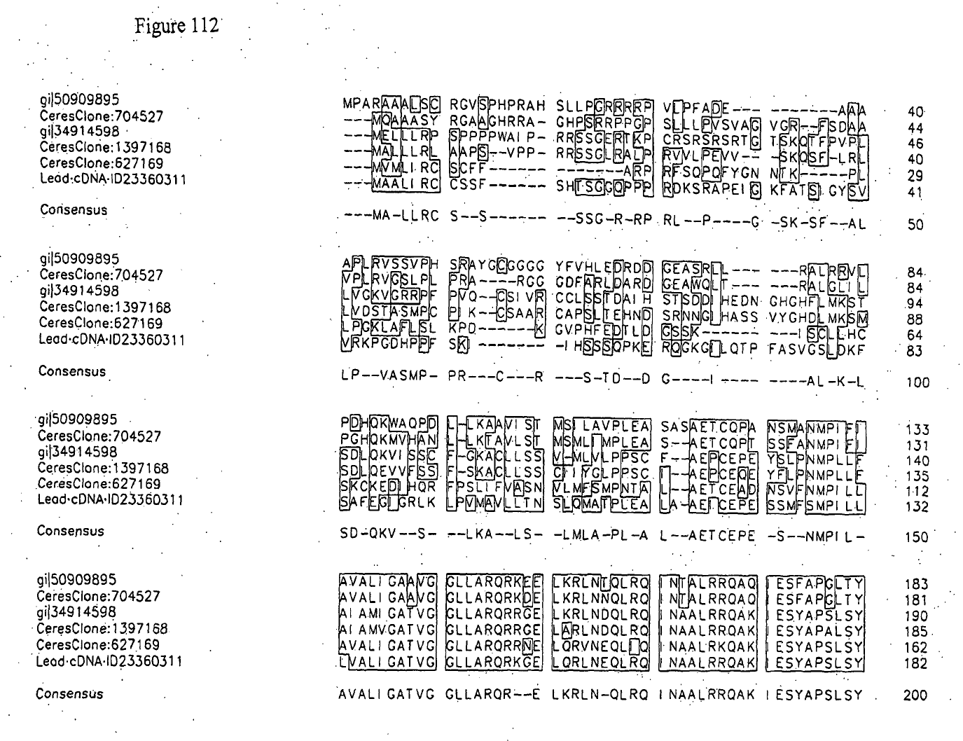

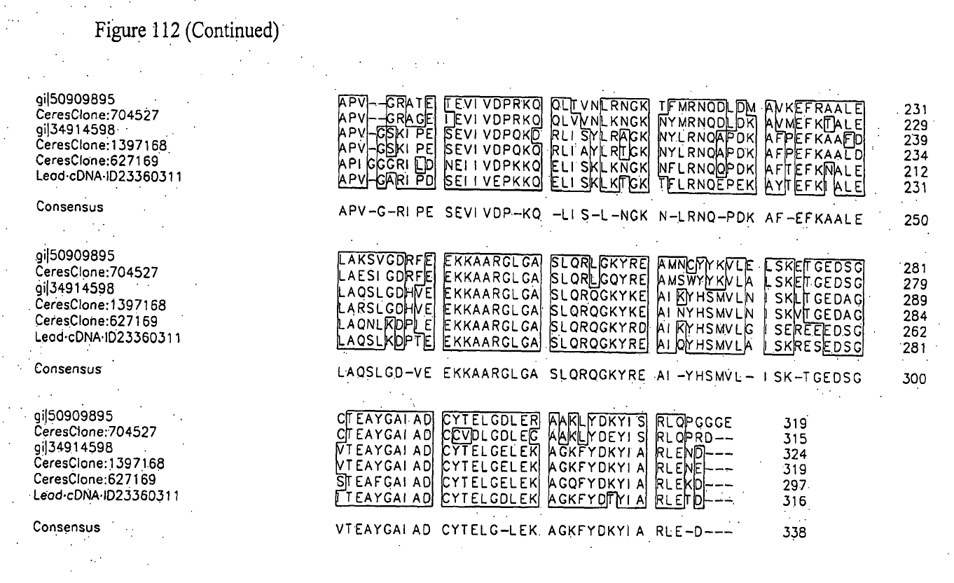

Amino acid sequences of homologs and/or orthologs of the polypeptide having the amino acid sequence set forth in SEQ ID NO:376 and SEQ ID NO:1158 are provided in FIG. 33 and FIG. 112, respectively. Each of FIG. 33 and FIG. 112 also includes a consensus amino acid sequence determined by aligning homologous and/or orthologous amino acid sequences with the amino acid sequence set forth in SEQ ID NO:376 or SEQ ID NO:1158, respectively.

For example, the alignment in FIG. 33 provides the amino acid sequences of cDNA ID 23662829 (SEQ ID NO:376), CeresClone:12573 (SEQ ID NO:377), and CeresClone:246144 (SEQ ID NO:380). Other homologs and/or orthologs of SEQ ID NO:376 include Public GI no. 21537266 (SEQ ID NO:378) and Public GI no. 7269949 (SEQ ID NO:379).

The alignment in FIG. 112 provides the amino acid sequences of cDNA ID 23360311 (SEQ ID NO:1158), CeresClone:627169 (SEQ ID NO:1159), gi|34914598 (SEQ ID NO:1160), CeresClone:1397168 (SEQ ID NO:1161), gi|50909895 (SEQ ID NO:1162), and CeresClone:704527 (SEQ ID NO:1163).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:377-380, SEQ ID NOs:1159-1163, or the consensus sequences set forth in FIG. 33 or FIG. 112.

A regulatory protein can have an FHA domain. The FHA (forkhead-associated) domain is a phosphopeptide recognition domain found in many regulatory proteins. It displays specificity for phosphothreonine-containing epitopes but will also recognize phosphotyrosine with relatively high affinity. The FHA domain spans approximately 80-100 amino acid residues folded into an eleven-stranded beta sandwich, which sometimes contains small helical insertions between the loops connecting the strands. Genes encoding FHA-containing proteins have been identified in eubacterial and eukaryotic but not archaeal genomes. The FHA domain is present in a diverse range of proteins, such as kinases, phosphatases, kinesins, transcription factors, RNA binding proteins, and metabolic enzymes involved in many different cellular processes, such as DNA repair, signal transduction, vesicular transport, and protein degradation. SEQ ID NO:664 and SEQ ID NO:760 set forth the amino acid sequences of DNA clones, identified herein as cDNA ID 24380616 (SEQ ID NO:663) and cDNA ID 23760303 (SEQ ID NO:759), each of which is predicted to encode a polypeptide having an FHA domain.

A regulatory protein can comprise the amino acid sequence set forth in SEQ ID NO:664 or SEQ ID NO:760. Alternatively, a regulatory protein can be a homolog, ortholog, or variant of the polypeptide having the amino acid sequence set forth in SEQ ID NO:664 or SEQ ID NO:760. For example, a regulatory protein can have an amino acid sequence with at least 60% sequence identity, e.g., 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:664 or SEQ ID NO:760.

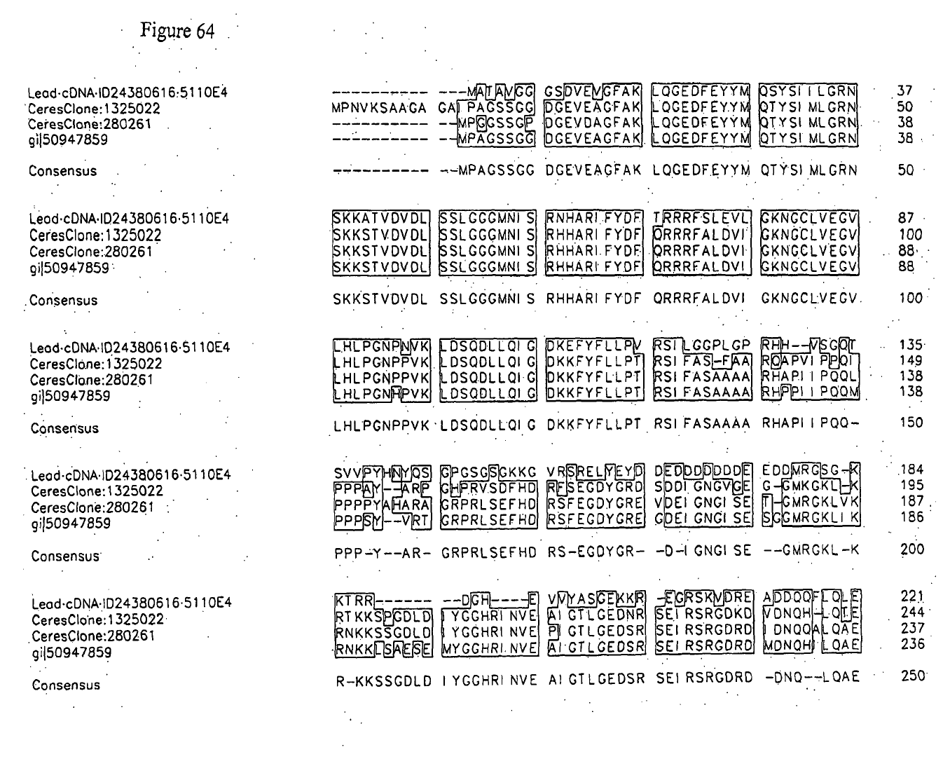

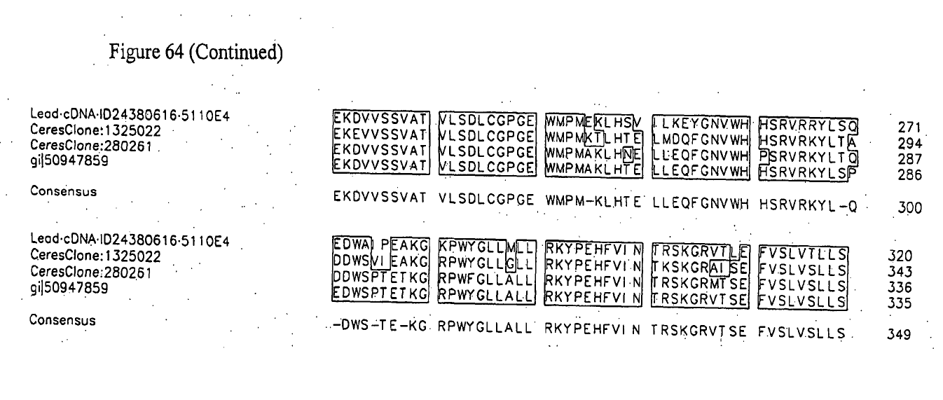

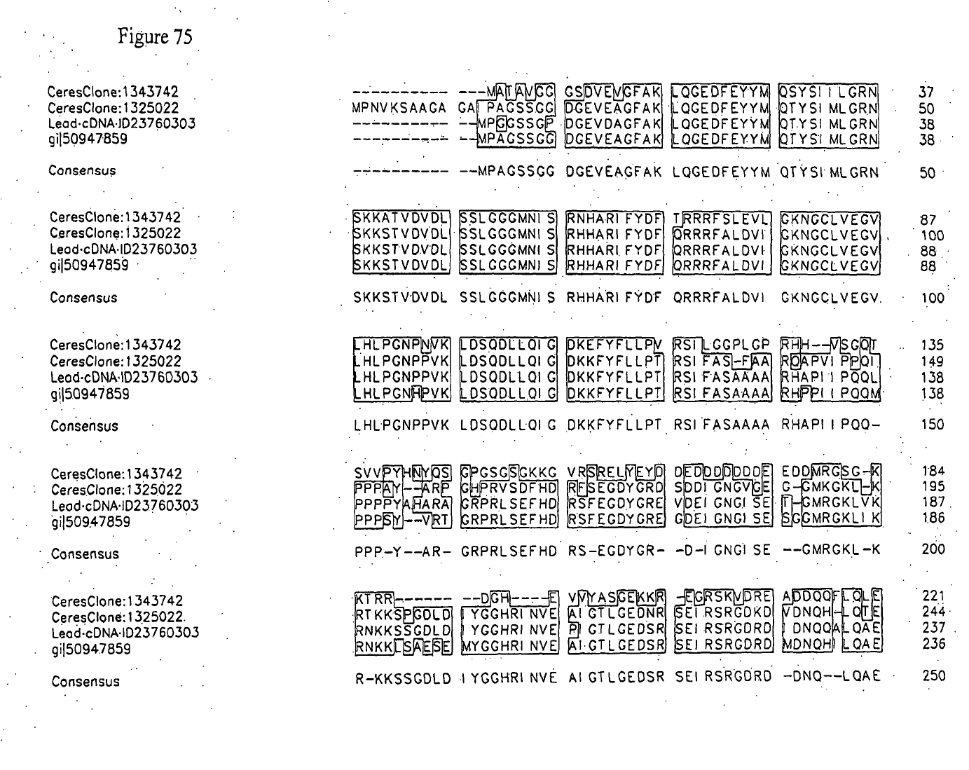

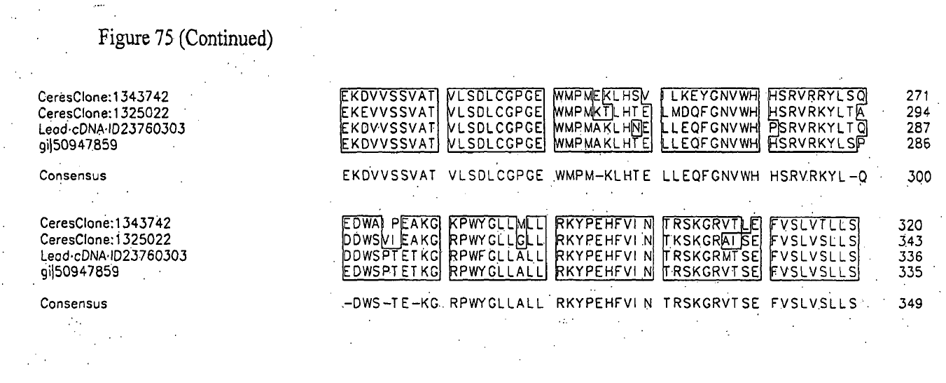

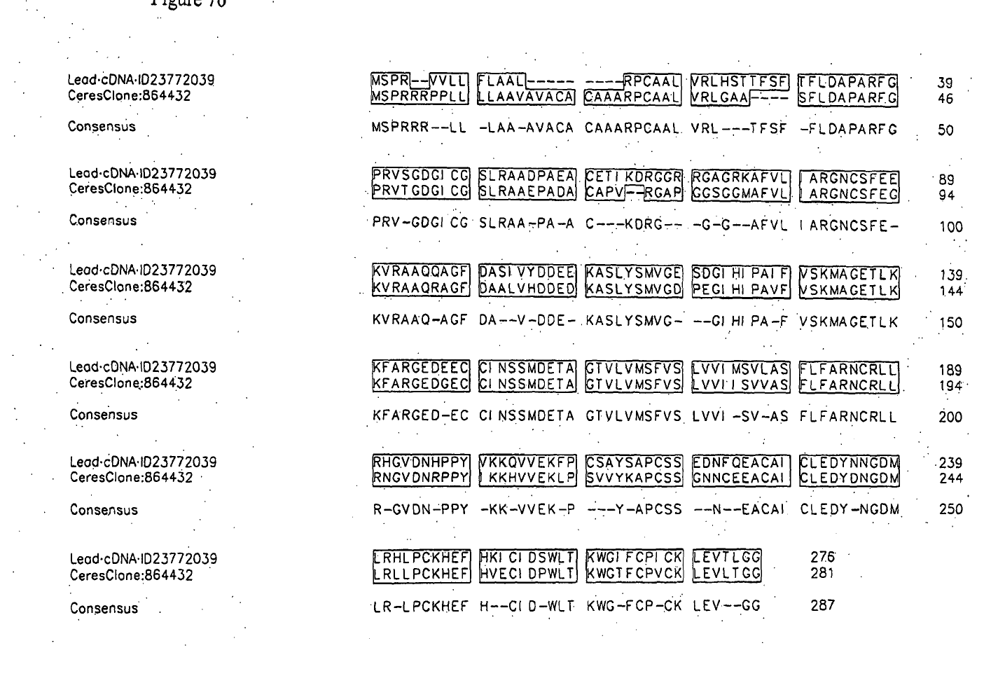

Amino acid sequences of homologs and/or orthologs of the polypeptide having the amino acid sequence set forth in SEQ ID NO:664 and SEQ ID NO:760 are provided in FIG. 64 and FIG. 75, respectively. Each of FIG. 64 and FIG. 75 includes a consensus amino acid sequence determined by aligning homologous and/or orthologous amino acid sequences with the amino acid sequence set forth in SEQ ID NO:664 or SEQ ID NO:760, respectively.

For example, the alignment in FIG. 64 provides the amino acid sequences of cDNA ID 24380616 (5110E4; SEQ ID NO:664), CeresClone:280261 (SEQ ID NO:665), gi|50947859 (SEQ ID NO:666), and CeresClone:1325022 (SEQ ID NO:669). Other homologs and/or orthologs of SEQ ID NO:664 include Public GI no. 51965036 (SEQ ID NO:667) and Ceres CLONE ID no. 365048 (SEQ ID NO:668).

The alignment in FIG. 75 provides the amino acid sequences of cDNA ID 23760303 (SEQ ID NO:760), gi|50947859 (SEQ ID NO:761), CeresClone:1325022 (SEQ ID NO:763), and CeresClone:1343742 (SEQ ID NO:764). Other homologs and/or orthologs of SEQ ID NO:760 include Public GI no. 51965036 (SEQ ID NO:762).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:665-669, SEQ ID NOs:761-764, or the consensus sequences set forth in FIG. 64 or FIG. 75.

A regulatory protein can contain an ankyrin repeat. The ankyrin repeat is one of the most common protein-protein interaction motifs in nature. Ankyrin repeats are tandemly repeated modules of about 33 amino acids. The repeat has been found in proteins of diverse function such as transcriptional initiators, cell-cycle regulators, cytoskeletal, ion transporters and signal transducers. Each repeat folds into a helix-loop-helix structure with a beta-hairpin/loop region projecting out from the helices at a 90 degree angle. The repeats stack together to form an L-shaped structure.

In some cases, a regulatory protein can contain an ankyrin repeat and a BTB/POZ domain. The BTB (for BR-C, ttk and bab) or POZ (for Pox virus and zinc finger) domain is present near the N-terminus of a fraction of zinc finger (zf-C2H2) proteins and is also found in proteins that contain the Kelch_{—}1 motif. The BTB/POZ domain mediates homomeric dimerization and, in some instances, heteromeric dimerization. The structure of the dimerized PLZF BTB/POZ domain consists of a tightly intertwined homodimer. The central scaffolding of the protein is made up of a cluster of alpha-helices flanked by short beta-sheets at both the top and bottom of the molecule. POZ domains from several zinc finger proteins have been shown to mediate transcriptional repression and to interact with components of histone deacetylase co-repressor complexes including N-CoR and SMRT. The POZ or BTB domain is also known as BR-C/Ttk or ZiN. SEQ ID NO:1297 sets forth the amino acid sequence of a DNA clone, identified herein as cDNA ID 23380202 (SEQ ID NO:1296), that is predicted to encode a polypeptide containing an ankyrin repeat and a BTB/POZ domain.

In some cases, a regulatory protein can contain an ankyrin repeat and an IQ calmodulin-binding motif. Calmodulin (CaM) is recognized as a major calcium sensor that orchestrates regulatory events through interaction with a diverse group of cellular proteins. Many CaM binding proteins contain three classes of recognition motifs: the IQ motif, which is a consensus sequence for Ca²⁺-independent binding, and two related motifs for Ca²⁺-dependent binding. SEQ ID NO:1210 sets forth the amino acid sequence of a DNA clone, identified herein as cDNA ID 23694932 (SEQ ID NO:1209), that is predicted to encode a polypeptide containing an ankyrin repeat and an IQ calmodulin-binding motif.

A regulatory protein can comprise the amino acid sequence set forth in SEQ ID NO:1210. Alternatively, a regulatory protein can be a homolog, ortholog, or variant of the polypeptide having the amino acid sequence set forth in SEQ ID NO:1210. For example, a regulatory protein can have an amino acid sequence with at least 35% sequence identity, e.g., 36%, 40%, 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:1210.

A regulatory protein can contain a zf-MYND, or MYND finger, domain and a SET domain. The MYND (myeloid, Nervy, and DEAF-1) domain is present in a group of proteins that includes RP-8 (PDCD2), Nervy, and predicted proteins from Drosophila, mammals, Caenorhabditis elegans, yeast, and plants. The MYND domain consists of a cluster of invariantly spaced cysteine and histidine residues that form a potential zinc-binding motif. Mutating conserved cysteine residues in the DEAF-1 MYND domain does not abolish DNA binding, which suggests that the MYND domain might be involved in protein-protein interactions. Indeed, the MYND domain of ETO/MTG8 interacts directly with the N-CoR and SMRT co-repressors. The MYND motif in mammalian polypeptides appears to constitute a protein-protein interaction domain that functions as a co-repressor-recruiting interface. SET domains, consisting of about 130 amino acids, also appear to be protein-protein interaction domains. It has been demonstrated that SET domains mediate interactions with a family of proteins that display similarity with dual-specificity phosphatases (dsPTPases). Polypeptides bearing the widely distributed SET domain have been shown to contribute to epigenetic mechanisms of gene regulation by methylation of lysine residues in histones and other proteins. A subset of SET domains have been called PR domains. These domains are divergent in sequence from other SET domains, but also appear to mediate protein-protein interactions. SEQ ID NO:674 sets forth the amino acid sequence of a DNA clone, identified herein as cDNA ID 23467433 (SEQ ID NO:673), that is predicted to encode a polypeptide containing a zf-MYND and a SET domain.

A regulatory protein can comprise the amino acid sequence set forth in SEQ ID NO:674. Alternatively, a regulatory protein can be a homolog, ortholog, or variant of the polypeptide having the amino acid sequence set forth in SEQ ID NO:674. For example, a regulatory protein can have an amino acid sequence with at least 50% sequence identity, e.g., 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:674.

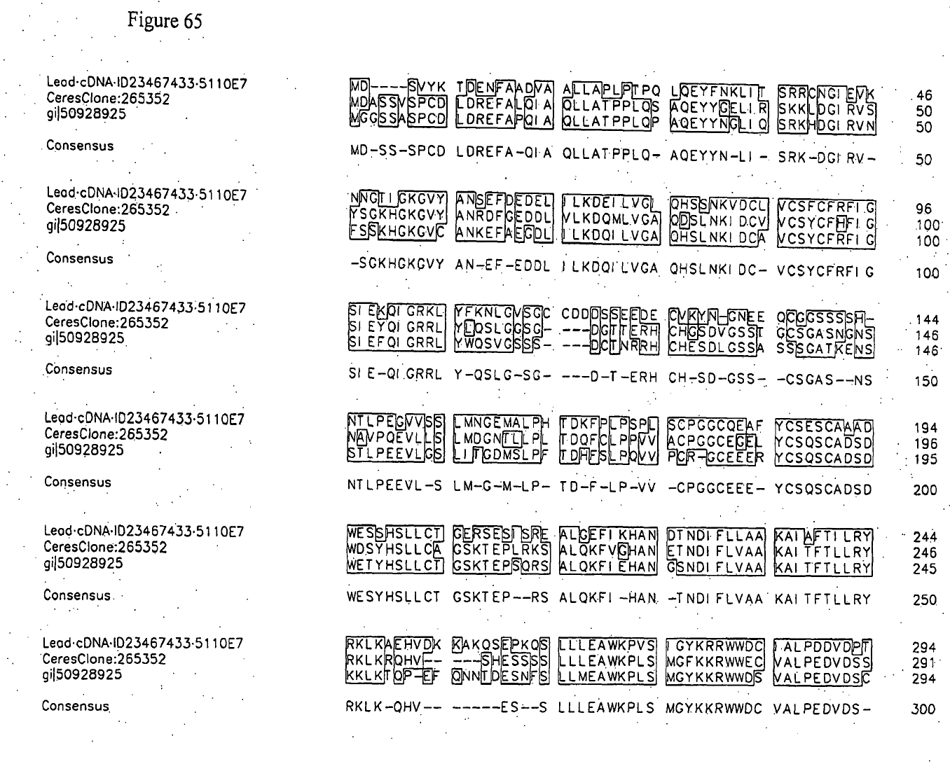

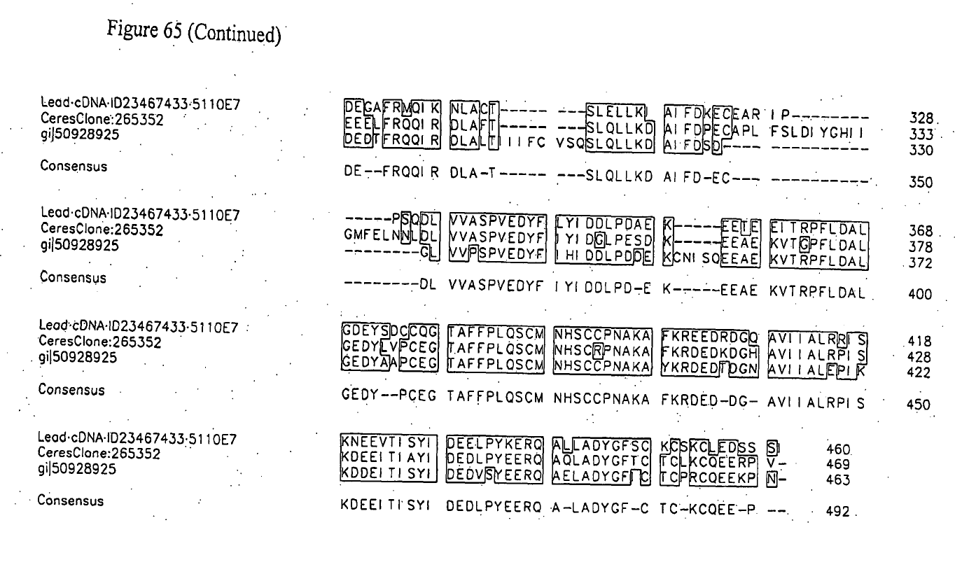

Amino acid sequences of homologs and/or orthologs of the polypeptide having the amino acid sequence set forth in SEQ ID NO:674 are provided in FIG. 65. FIG. 65 also includes a consensus amino acid sequence determined by aligning homologous and/or orthologous amino acid sequences with the amino acid sequence set forth in SEQ ID NO:674.

For example, the alignment in FIG. 65 provides the amino acid sequences of cDNA ID 23467433 (5110E7; SEQ ID NO:674), CeresClone:265352 (SEQ ID NO:676) and gi|50928925 (SEQ ID NO:677). Other homologs and/or orthologs of SEQ ID NO:674 include Public GI no. 62320769 (SEQ ID NO:675).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to SEQ ID NOs:675-677 or the consensus sequence set forth in FIG. 65.

A regulatory protein can contain a PHD domain. The homeodomain (PHD) finger is a C4HC3 zinc-finger-like motif found in nuclear proteins thought to be involved in chromatin-mediated transcriptional regulation. The PHD finger motif is reminiscent of, but distinct from, the C3HC4 type RING finger. Similar to the RING finger and the LIM domain, the PHD finger is thought to bind two zinc ions. The PHD finger could be involved in protein-protein interactions and assembly or activity of multicomponent complexes involved in transcriptional activation or repression. Alternatively, the interactions could be intra-molecular and important in maintaining the structural integrity of the protein. SEQ ID NO:309 sets forth the amino acid sequence of a DNA clone, referred to herein as cDNA ID 23370269 (SEQ ID NO:308), that is predicted to encode a PHD domain-containing polypeptide.

In some cases, a regulatory protein can contain a PHD domain and a putative zinc finger in N-recognin (zf-UBR1) domain. The putative zinc finger in N-recognin domain is a recognition component of the N-end rule pathway. The N-end rule-based degradation signal, which targets a protein for ubiquitin-dependent proteolysis, comprises a destabilizing amino-terminal residue and a specific internal lysine residue. SEQ ID NO:637 sets forth the amino acid sequence of a DNA clone, identified herein as cDNA ID 23503138 (SEQ ID NO:636), that is predicted to encode a polypeptide containing a PHD domain and a zf-UBR1 domain.

A regulatory protein can comprise the amino acid sequence set forth in SEQ ID NO:309 or SEQ ID NO:637. Alternatively, a regulatory protein can be a homolog, ortholog, or variant of the polypeptide having the amino acid sequence set forth in SEQ ID NO:309 or SEQ ID NO:637. For example, a regulatory protein can have an amino acid sequence with at least 60% sequence identity, e.g., 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:309 or SEQ ID NO:637.

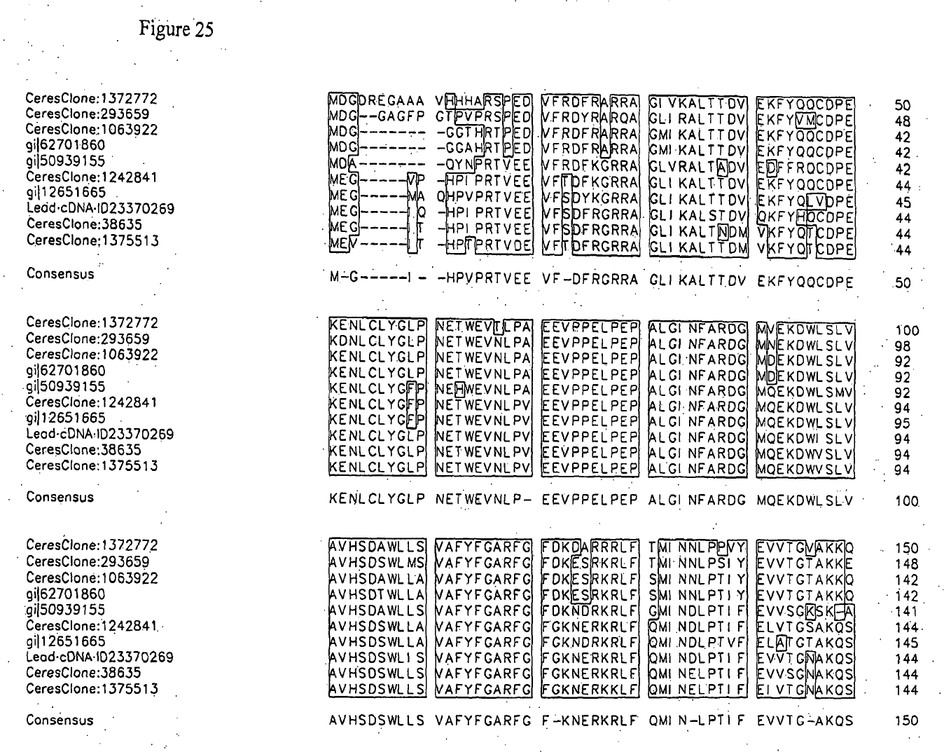

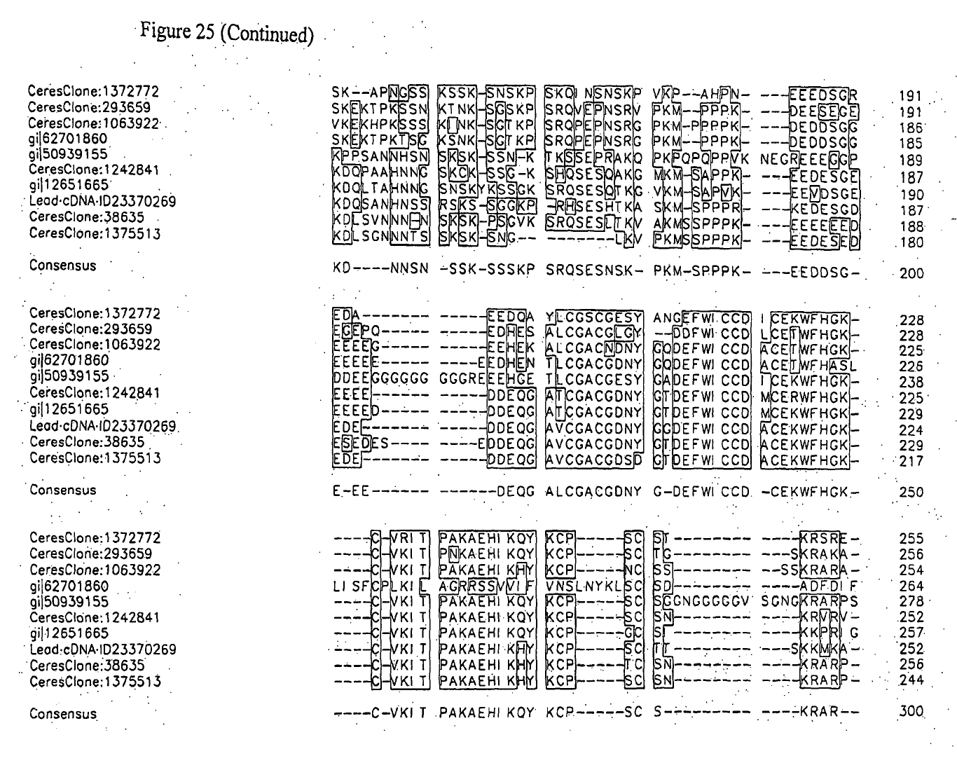

Amino acid sequences of homologs and/or orthologs of the polypeptide having the amino acid sequence set forth in SEQ ID NO:309 are provided in FIG. 25. FIG. 25 also includes a consensus amino acid sequence determined by aligning homologous and/or orthologous amino acid sequences with the amino acid sequence set forth in SEQ ID NO:309.

For example, the alignment in FIG. 25 provides the amino acid sequences of cDNA ID 23370269 (SEQ ID NO:309), CeresClone:38635 (SEQ ID NO:310), CeresClone:1375513 (SEQ ID NO:313), CeresClone:1242841 (SEQ ID NO:314), gi|12651665 (SEQ ID NO:315), gi|50939155 (SEQ ID NO:317), CeresClone:1063922 (SEQ ID NO:318), gi|62701860 (SEQ ID NO:319), CeresClone:293659 (SEQ ID NO:320), and CeresClone:1372772 (SEQ ID NO:321). Other homologs and/or orthologs of SEQ ID NO:309 include Public GI no. 21593407 (SEQ ID NO:311), Public GI no. 28827386 (SEQ ID NO:312), Public GI no. 14192880 (SEQ ID NO:316), Ceres CLONE ID no. 262186 (SEQ ID NO:322), and Ceres CLONE ID no. 484170 (SEQ ID NO:323).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:310-323 or the consensus sequence set forth in FIG. 25.

A regulatory protein can contain a Mov34 domain characteristic of a Mov34/MPN/PAD-1 family polypeptide. Mov34 polypeptides are reported to act as regulatory subunits of the 26 proteasome, which is involved in the ATP-dependent degradation of ubiquitinated proteins. Mov34 domains are found in the N-terminus of the proteasome regulatory subunits, eukaryotic initiation factor 3 (eIF3) subunits, and regulators of transcription factors. SEQ ID NO:158 and SEQ ID NO:387 set forth the amino acid sequences of DNA clones, identified herein as cDNA ID 24374230 (SEQ ID NO:157) and cDNA ID 23369491 (SEQ ID NO:386), respectively, each of which is predicted to encode a polypeptide containing a Mov34 domain.

A regulatory protein can comprise the amino acid sequence set forth in SEQ ID NO:158 or SEQ ID NO:387. Alternatively, a regulatory protein can be a homolog, ortholog, or variant of the polypeptide having the amino acid sequence set forth in SEQ ID NO:158 or SEQ ID NO:387. For example, a regulatory protein can have an amino acid sequence with at least 60% sequence identity, e.g., 60%, 65%, 70%, 75%, 80%, 85%, 90%, 95%, 97%, 98%, or 99% sequence identity, to the amino acid sequence set forth in SEQ ID NO:158 or SEQ ID NO:387.

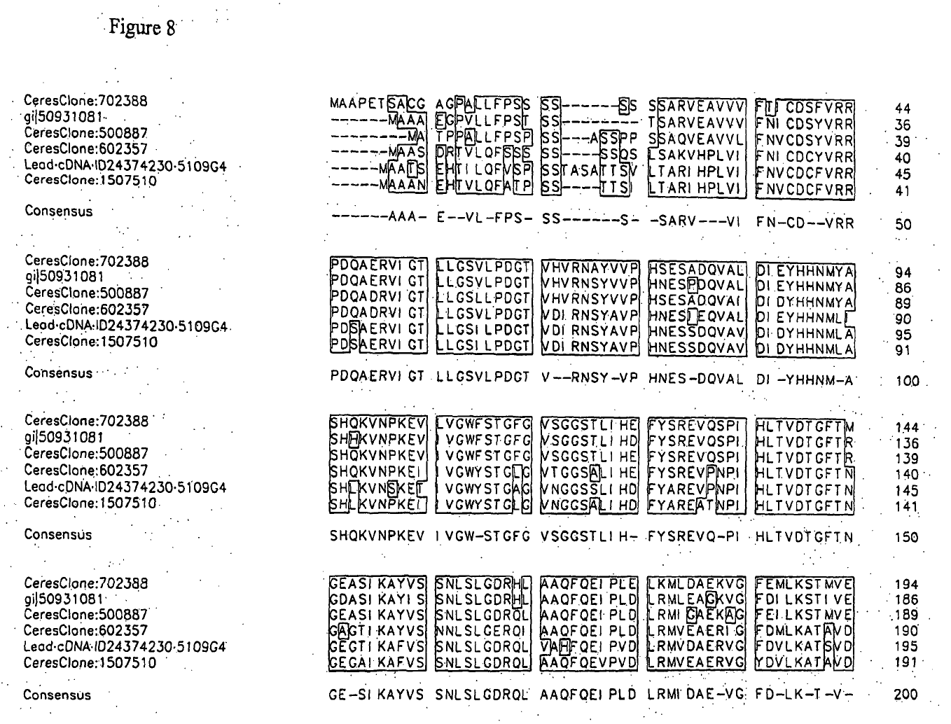

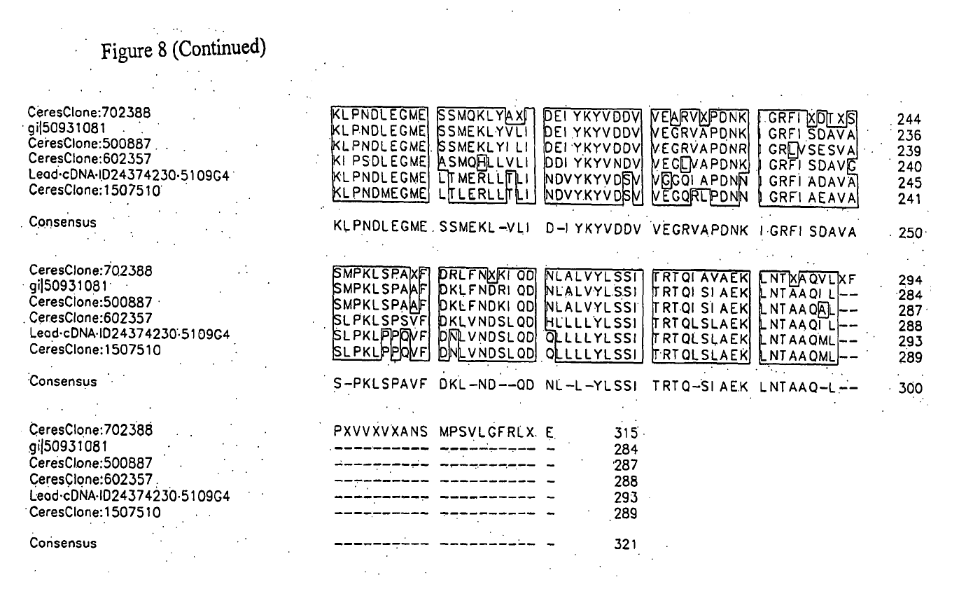

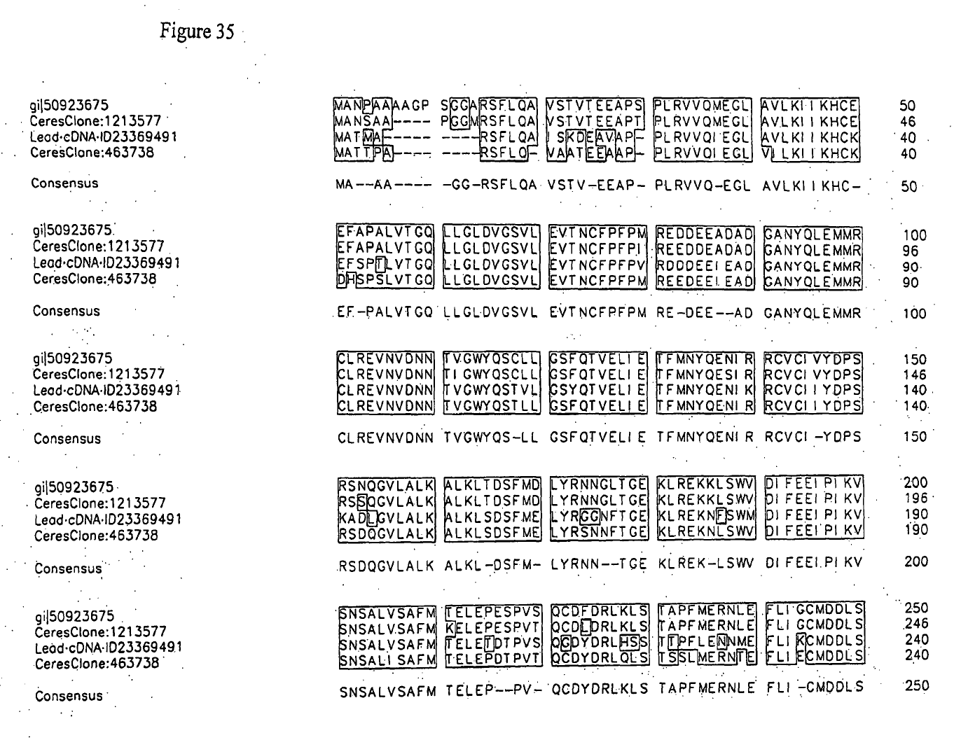

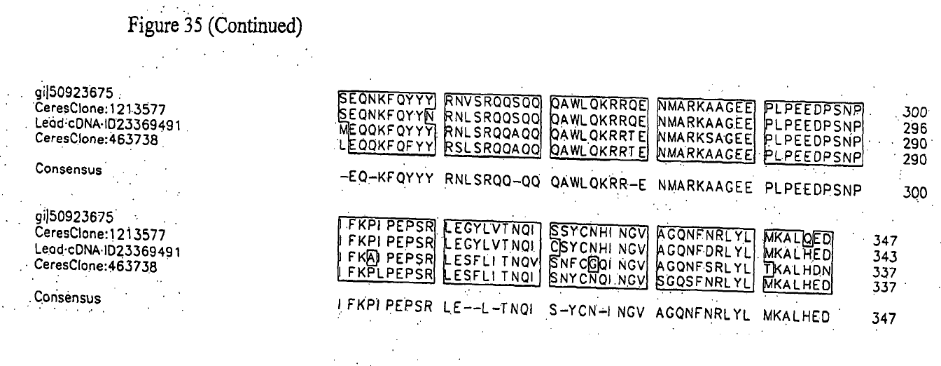

Amino acid sequences of homologs and/or orthologs of the polypeptide having the amino acid sequence set forth in SEQ ID NO:158 and SEQ ID NO:387 are provided in FIG. 8 and FIG. 35, respectively. Each of FIG. 8 and FIG. 35 also includes a consensus amino acid sequence determined by aligning homologous and/or orthologous amino acid sequences with the amino acid sequence set forth in SEQ ID NO:158 or SEQ ID NO:387, respectively.

For example, the alignment in FIG. 8 provides the amino acid sequences of cDNA ID 24374230 (5109G4; SEQ ID NO:158), CeresClone:1507510 (SEQ ID NO:159), CeresClone:602357 (SEQ ID NO:160), gi|50931081 (SEQ ID NO:163), CeresClone:500887 (SEQ ID NO:164), and CeresClone:702388 (SEQ ID NO:166). Other homologs and/or orthologs of SEQ ID NO:158 include Ceres CLONE ID no. 557575 (SEQ ID NO:161), Ceres CLONE ID no. 1119778 (SEQ ID NO:162), and Ceres CLONE ID no. 221299 (SEQ ID NO:165).

The alignment in FIG. 35 provides the amino acid sequences of cDNA ID 23369491 (SEQ ID NO:387), CeresClone:463738 (SEQ ID NO:388), gi|50923675 (SEQ ID NO:389), and CeresClone:1213577 (SEQ ID NO:390).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:159-166, SEQ ID NOs:388-390, or the consensus sequences set forth in FIG. 8 or FIG. 35.

A regulatory protein can contain a UCH domain characteristic of a ubiquitin carboxyl-terminal hydrolase polypeptide. Ubiquitin is highly conserved and commonly found conjugated to proteins in eukaryotic cells. Ubiquitin may act as a marker for rapid degradation, or it may have a chaperone function in protein assembly. The ubiquitin is released by cleavage from the bound protein by a protease. A number of deubiquitinating proteases are known, which are activated by thiol compounds and inhibited by thiol-blocking agents and ubiquitin aldehyde, and as such have the properties of cysteine proteases. SEQ ID NO:121 sets forth the amino acid sequence of a DNA clone, identified herein as cDNA ID 23548978 (SEQ ID NO:120), that is predicted to encode a polypeptide containing a UCH domain.

A regulatory protein can comprise the amino acid sequence set forth in SEQ ID NO:121. Alternatively, a regulatory protein can be a homolog, ortholog, or variant of the polypeptide having the amino acid sequence set forth in SEQ ID NO:121. For example, a regulatory protein can have an amino acid sequence with at least 40% sequence identity, e.g., 40%, 45%, 50%, 55%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:121.

A regulatory protein can have a DUF298 domain characteristic of a family of polypeptides containing a basic helix-loop-helix leucine zipper motif. The DUF298 domain is implicated in neddylation of the cullin 3 family and has a possible role in the regulation of the protein modifier Nedd8 E3 ligase. Neddylation is the process by which the C-terminal glycine of the ubiquitin-like protein Nedd8 is covalently linked to lysine residues in a protein through an isopeptide bond. SEQ ID NO:1404 sets forth the amino acid sequence of a DNA clone, identified herein as cDNA ID 23372744 (SEQ ID NO:1403), that is predicted to encode a polypeptide containing a DUF298 domain.

A regulatory protein can comprise the amino acid sequence set forth in SEQ ID NO:1404. Alternatively, a regulatory protein can be a homolog, ortholog, or variant of the polypeptide having the amino acid sequence set forth in SEQ ID NO:1404. For example, a regulatory protein can have an amino acid sequence with at least 55% sequence identity, e.g., 55%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:1404.

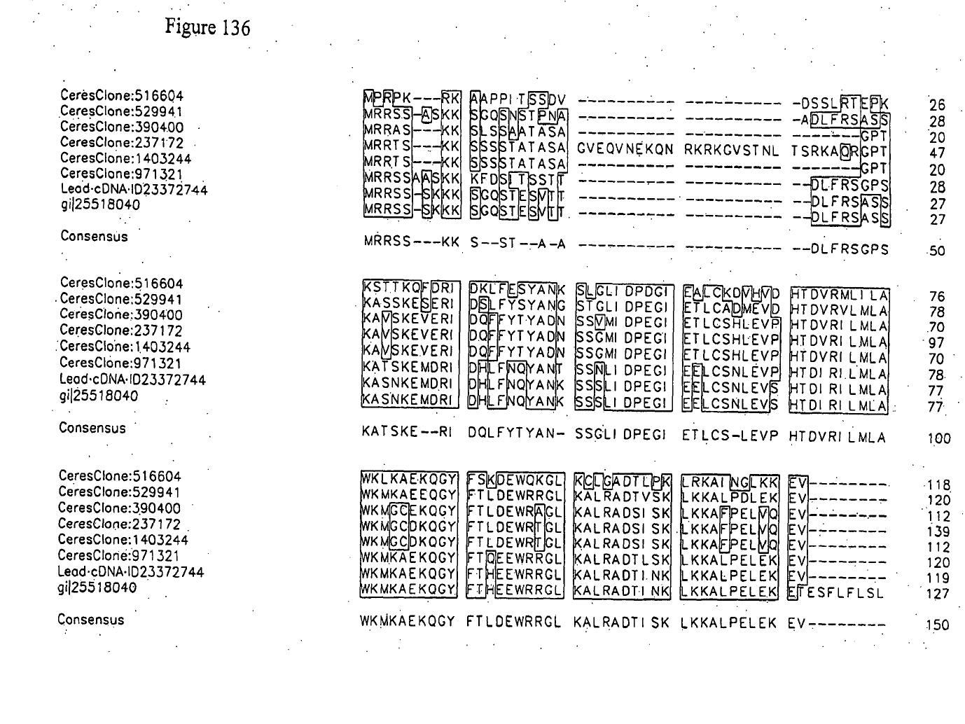

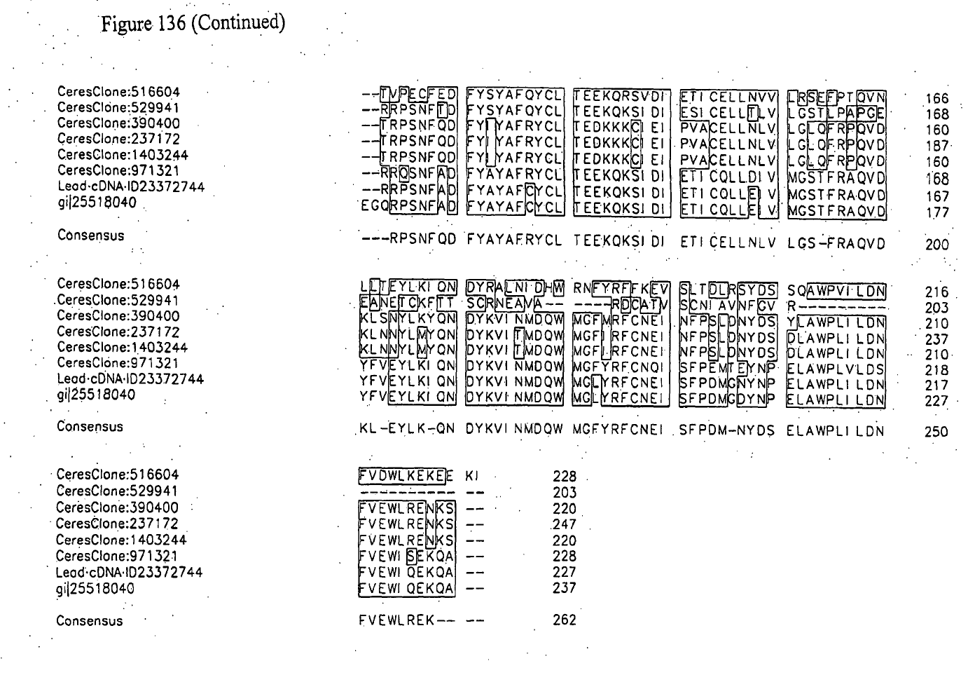

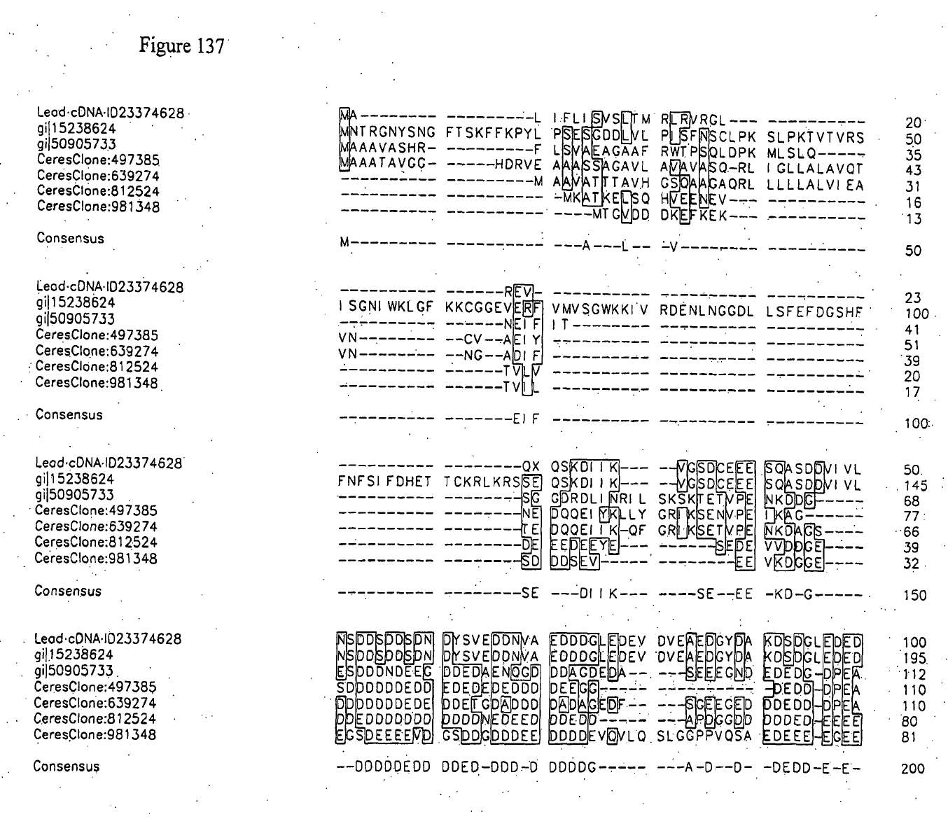

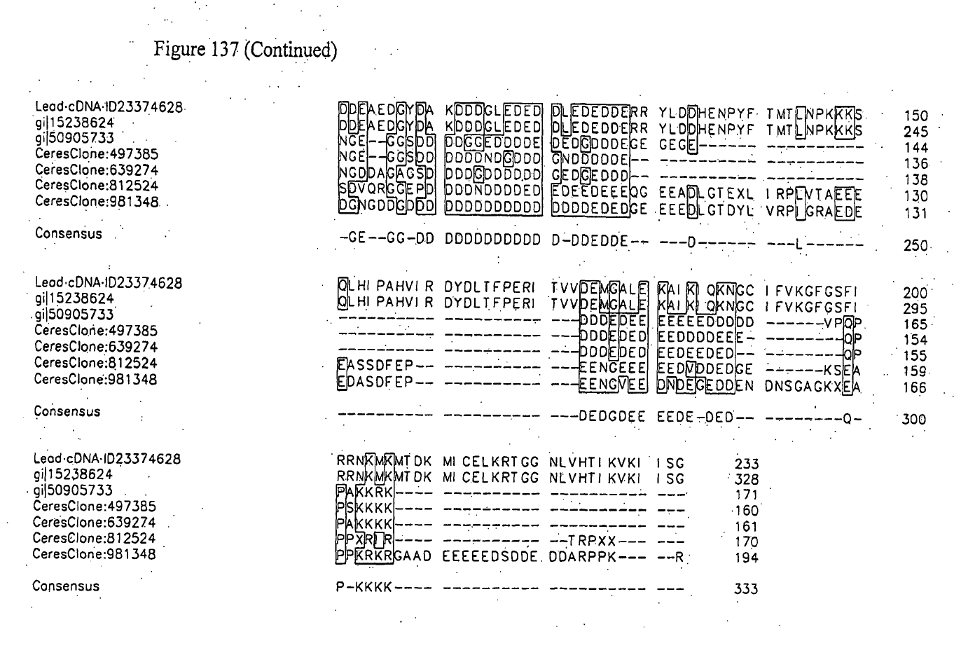

Amino acid sequences of homologs and/or orthologs of the polypeptide having the amino acid sequence set forth in SEQ ID NO:1404 are provided in FIG. 136. FIG. 136 also includes a consensus amino acid sequence determined by aligning homologous and/or orthologous amino acid sequences with the amino acid sequence set forth in SEQ ID NO:1404.

For example, the alignment in FIG. 136 provides the amino acid sequences of cDNA ID 23372744 (SEQ ID NO:1404), gi|25518040 (SEQ ID NO:1405), CeresClone:971321 (SEQ ID NO:1406), CeresClone:529941 (SEQ ID NO:1407). CeresClone:390400 (SEQ ID NO:1408), CeresClone:237172 (SEQ ID NO:1409), CeresClone:1403244 (SEQ ID NO:1410), and CeresClone:516604 (SEQ ID NO:1411).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:1405-1411 or the consensus sequence set forth in FIG. 136.

A regulatory protein can contain a CCT motif. The CCT (CONSTANS, CO-like, and TOC1) domain is a highly conserved basic module of about 43 amino acids, which is often found near the C-terminus of plant proteins involved in light signal transduction. The CCT domain is found in association with other domains, such as the B-box zinc finger, the GATA-type zinc finger, the ZIM motif or the response regulatory domain. The CCT domain contains a putative nuclear localization signal, has been shown to be involved in nuclear localization, and probably also has a role in protein-protein interaction. SEQ ID NO:1019 sets forth the amino acid sequence of a DNA clone, identified herein as cDNA ID 23385230 (SEQ ID NO:1018), that is predicted to encode a polypeptide containing a CCT motif.

A regulatory protein can comprise the amino acid sequence set forth in SEQ ID NO:1019. Alternatively, a regulatory protein can be a homolog, ortholog, or variant of the polypeptide having the amino acid sequence set forth in SEQ ID NO:1019. For example, a regulatory protein can have an amino acid sequence with at least 55% sequence identity, e.g., 55%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:1019.

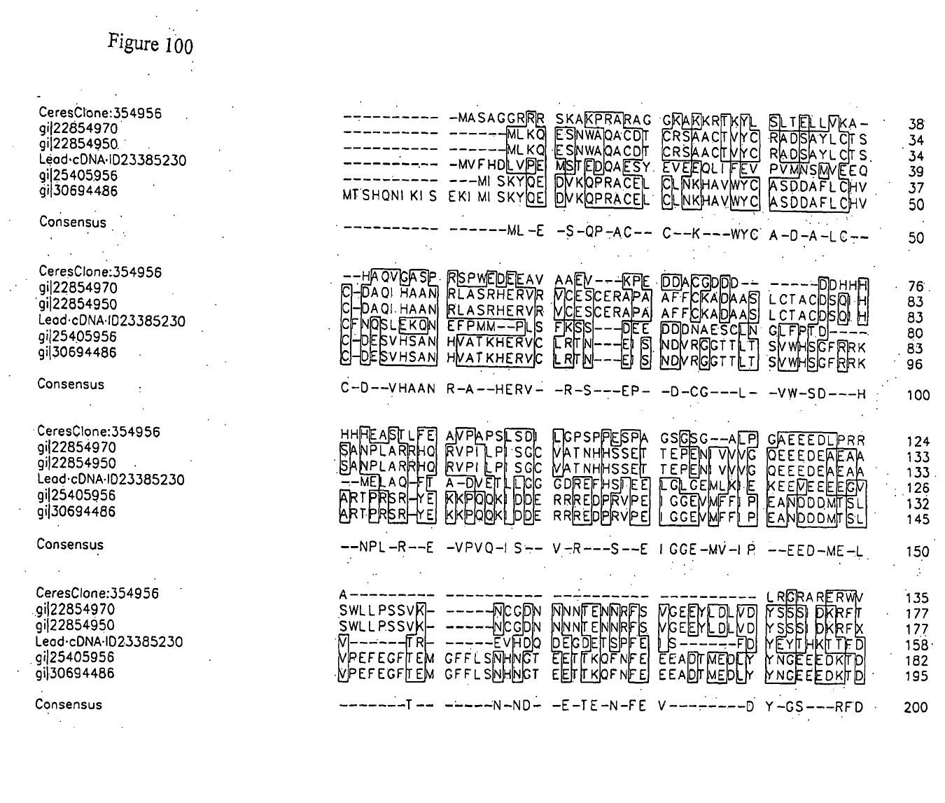

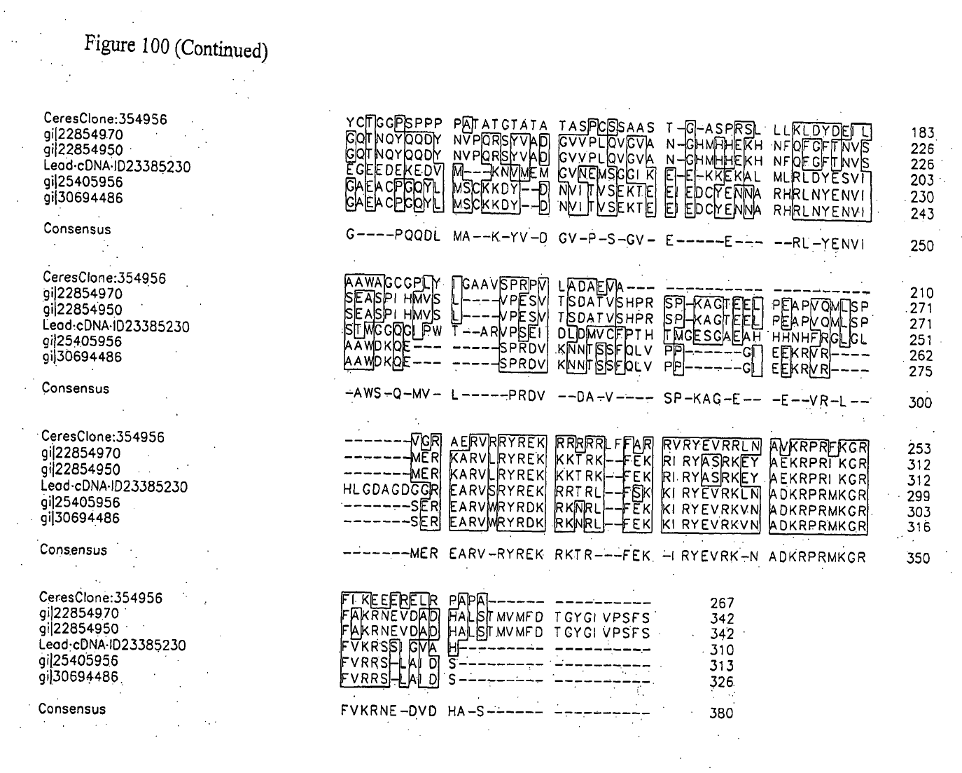

Amino acid sequences of homologs and/or orthologs of the polypeptide having the amino acid sequence set forth in SEQ ID NO:1019 are provided in FIG. 100. FIG. 100 also includes a consensus amino acid sequence determined by aligning homologous and/or orthologous amino acid sequences with the amino acid sequence set forth in SEQ ID NO:1019.

For example, the alignment in FIG. 100 provides the amino acid sequences of cDNA ID 23385230 (SEQ ID NO:1019), gi|25405956 (SEQ ID NO:1020), gi|30694486 (SEQ ID NO:1021), CeresClone:354956 (SEQ ID NO:1022), gi|22854970 (SEQ ID NO:1023), and gi|22854950 (SEQ ID NO:1024).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:1020-1024 or the consensus sequence set forth in FIG. 100.

A regulatory protein can contain one or more domains characteristic of a DNA repair polypeptide. For example, a regulatory protein can contain an HhH-GPD domain and an OGG_N domain. The HhH-GPD domain is characteristic of an HhH-GPD superfamily base excision DNA repair polypeptide. The name of the HhH-GPD domain is derived from the hallmark helix-hairpin-helix and Gly/Pro rich loop followed by a conserved aspartate. The HhH-GPD domain is found in a diverse range of structurally related DNA repair proteins that include endonuclease III and DNA glycosylase MutY, an A/G-specific adenine glycosylase. The HhH-GPD family also includes DNA-3-methyladenine glycosylase II, 8-oxoguanine DNA glycosylases, and other members of the AlkA family. The OGG_N domain, which is organized into a single copy of a TBP-like fold, is found in the N-terminus of 8-oxoguanine DNA glycosylase, the enzyme responsible for the process which leads to the removal of 8-oxoguanine residues from DNA. The 8-oxoguanine DNA glycosylase enzyme has DNA glycosylase and DNA lyase activity. SEQ ID NO:851 sets forth the amino acid sequence of a DNA clone, identified herein as cDNA ID 23486285 (SEQ ID NO:850), that is predicted to encode a polypeptide having an HhH-GPD domain and an OGG_N domain.

A regulatory protein can comprise the amino acid sequence set forth in SEQ ID NO:851. Alternatively, a regulatory protein can be a homolog, ortholog, or variant of the polypeptide having the amino acid sequence set forth in SEQ ID NO:851. For example, a regulatory protein can have an amino acid sequence with at least 55% sequence identity, e.g., 55%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:851.

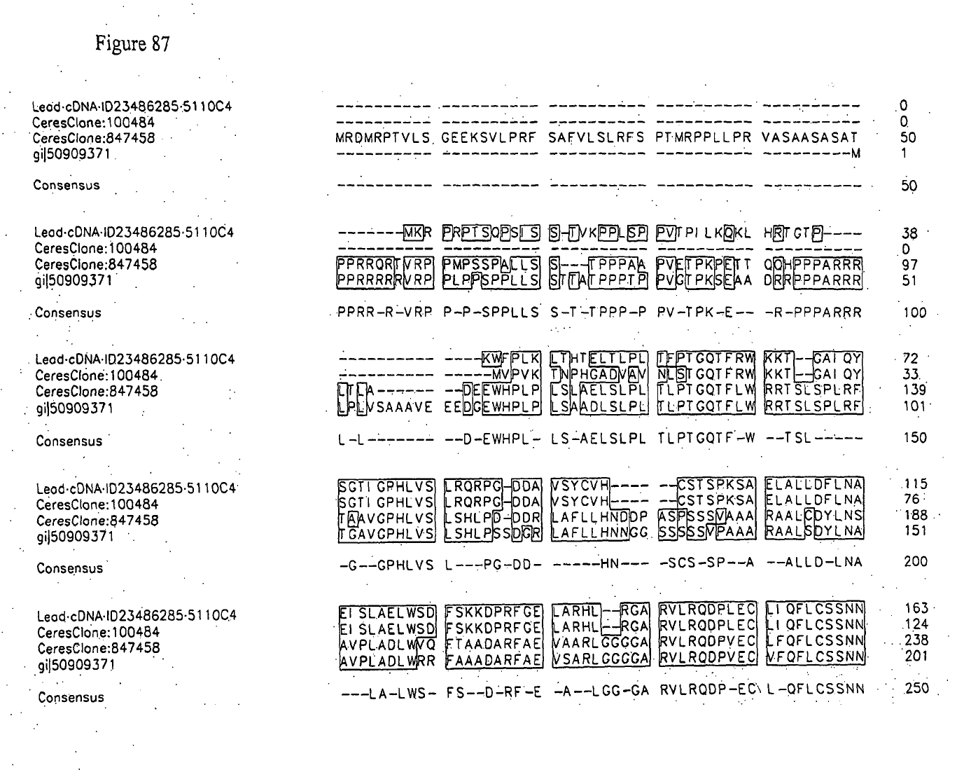

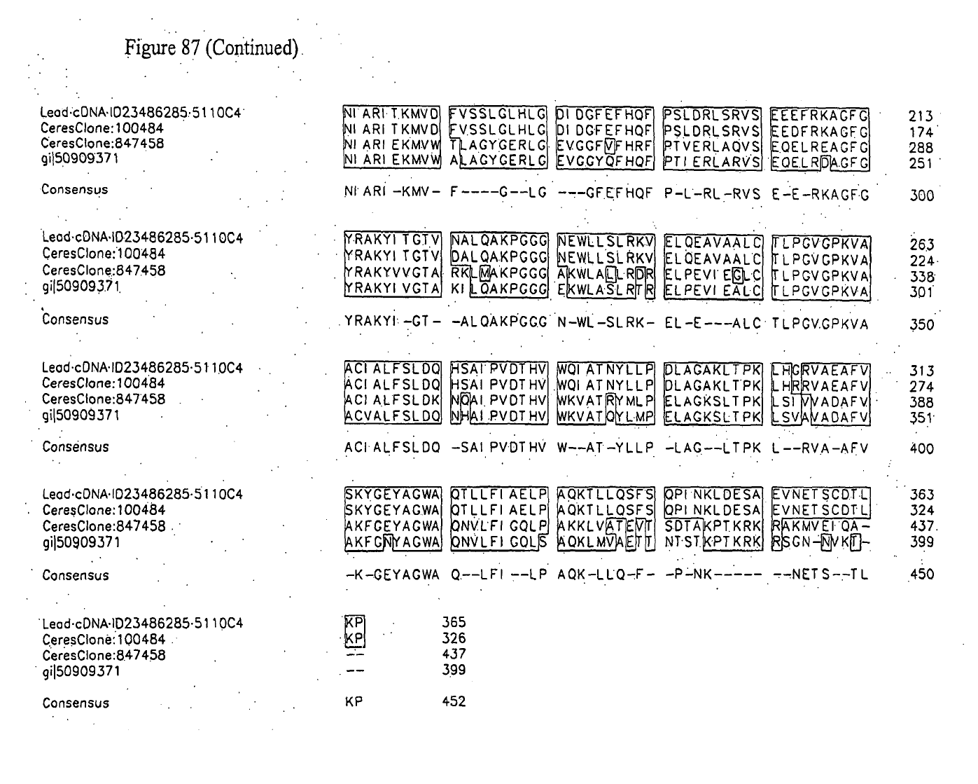

Amino acid sequences of homologs and/or orthologs of the polypeptide having the amino acid sequence set forth in SEQ ID NO:851 are provided in FIG. 87. FIG. 87 also includes a consensus amino acid sequence determined by aligning homologous and/or orthologous amino acid sequences with the amino acid sequence set forth in SEQ ID NO:851.

For example, the alignment in FIG. 87 provides the amino acid sequences of cDNA ID 23486285 (5110C4; SEQ ID NO:851), CeresClone:100484 (SEQ ID NO:852), CeresClone:847458 (SEQ ID NO:853), and gi|50909371 (SEQ ID NO:854).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:852-854 or the consensus sequence set forth in FIG. 87.

A regulatory protein can contain an SSB domain characteristic of a polypeptide belonging to the single-strand binding protein family. The SSB family includes single stranded binding proteins and also the primosomal replication protein N (PriB). The Escherichia coli single-strand binding protein (gene ssb), also known as the helix-destabilizing protein, binds tightly, as a homotetramer, to single-stranded DNA and plays an important role in DNA replication, recombination and repair. SEQ ID NO:845 sets forth the amino acid sequence of a DNA clone, identified herein as cDNA ID 23492765 (SEQ ID NO:844), that is predicted to encode a polypeptide containing an SSB domain.

A regulatory protein can comprise the amino acid sequence set forth in SEQ ID NO:845. Alternatively, a regulatory protein can be a homolog, ortholog, or variant of the polypeptide having the amino acid sequence set forth in SEQ ID NO:845. For example, a regulatory protein can have an amino acid sequence with at least 50% sequence identity, e.g., 50%, 55%, 60%, 65%, 67%, 68%, 70%, 75%, 80%, 85%, 90%, 95%, 97%, 98%, or 99% sequence identity, to the amino acid sequence set forth in SEQ ID NO:845.

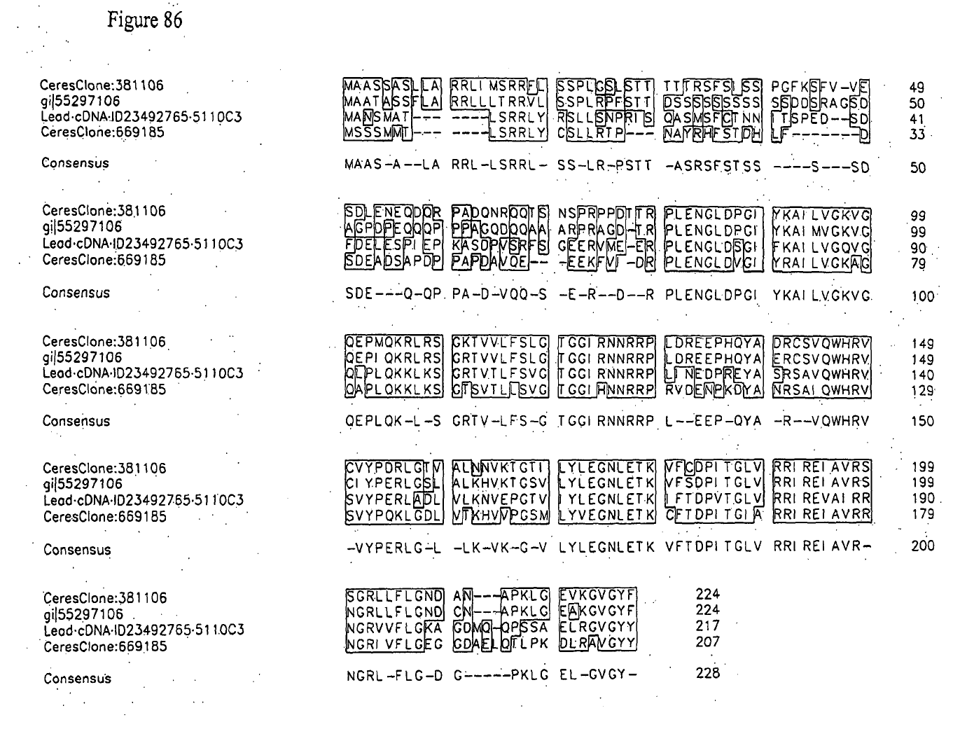

Amino acid sequences of homologs and/or orthologs of the polypeptide having the amino acid sequence set forth in SEQ ID NO:845 are provided in FIG. 86. FIG. 86 also includes a consensus amino acid sequence determined by aligning homologous and/or orthologous amino acid sequences with the amino acid sequence set forth in SEQ ID NO:845.

For example, the alignment in FIG. 86 provides the amino acid sequences of cDNA ID 23492765 (5110C3; SEQ ID NO:845), CeresClone:669185 (SEQ ID NO:846), CeresClone:381106 (SEQ ID NO:847), and gi|55297106 (SEQ ID NO:848). Other homologs and/or orthologs of SEQ ID NO:845 include Public GI no. 34911652 (SEQ ID NO:849).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:846-849 or the consensus sequence set forth in FIG. 86.

A regulatory protein can have a ParB-like nuclease (ParBc) domain. Proteins containing the ParBc domain appear to be related to the Escherichia coli plasmid protein ParB, which preferentially cleaves single-stranded DNA. ParB also nicks supercoiled plasmid DNA preferably at sites with potential single-stranded character, such as AT-rich regions and sequences that can form cruciform structures. ParB also exhibits 5′ to 3′ exonuclease activity. SEQ ID NO:593 sets forth the amino acid sequence of a DNA clone, identified herein as cDNA ID 23553534 (SEQ ID NO:592), that is predicted to encode a polypeptide containing a ParBc domain.

A regulatory protein can comprise the amino acid sequence set forth in SEQ ID NO:593. Alternatively, a regulatory protein can be a homolog, ortholog, or variant of the polypeptide having the amino acid sequence set forth in SEQ ID NO:593. For example, a regulatory protein can have an amino acid sequence with at least 65% sequence identity, e.g., 65%, 67%, 68%, 70%, 75%, 80%, 85%, 90%, 95%, 97%, 98%, or 99% sequence identity, to the amino acid sequence set forth in SEQ ID NO:593.

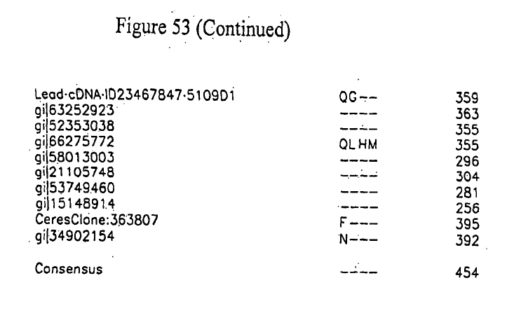

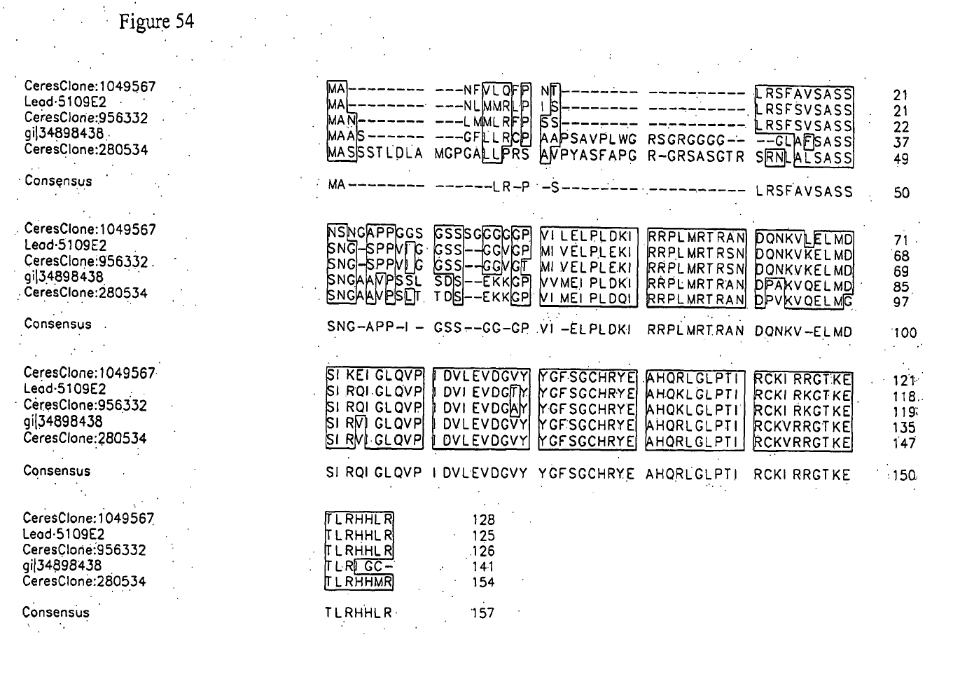

Amino acid sequences of homologs and/or orthologs of the polypeptide having the amino acid sequence set forth in SEQ ID NO:593 are provided in FIG. 54. FIG. 54 also includes a consensus amino acid sequence determined by aligning homologous and/or orthologous amino acid sequences with the amino acid sequence set forth in SEQ ID NO:593.

For example, the alignment in FIG. 54 provides the amino acid sequences of cDNA ID 23553534 (SEQ ID NO:593), CeresClone:956332 (SEQ ID NO:594), CeresClone:1049567 (SEQ ID NO:595), gi|34898438 (SEQ ID NO:596), and CeresClone:280534 (SEQ ID NO:597).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:594-597 or the consensus sequence set forth in FIG. 54.

A regulatory protein can contain a Ras domain characteristic of a Ras family polypeptide. Most of the members of the Ras superfamily have GTPase activity and some of the members have been implicated in various processes including cell development, cell and tissue differentiation, growth, survival, cytokine production, and vesicle-trafficking. The small Ras-GTPases are involved in intracellular cell signaling transduction pathway leading to modulation of gene expression, thus affecting the various processes mentioned above. SEQ ID NO:95 and SEQ ID NO:392 set forth the amino acid sequences of DNA clones, identified herein as cDNA ID 23693590 (SEQ ID NO:94) and cDNA ID 23384563 (SEQ ID NO:391), respectively, each of which is predicted to encode a polypeptide containing a Ras domain.

A regulatory protein can comprise the amino acid sequence set forth in SEQ ID NO:95 or SEQ ID NO:392. Alternatively, a regulatory protein can be a homolog, ortholog, or variant of the polypeptide having the amino acid sequence set forth in SEQ ID NO:95 or SEQ ID NO:392. For example, a regulatory protein can have an amino acid sequence with at least 50% sequence identity, e.g., 50%, 55%, 60%, 65%, 70%, 75%, 80%, 85%, 90%, 95%, 97%, 98%, or 99% sequence identity, to the amino acid sequence set forth in SEQ ID NO:95 or SEQ ID NO:392.

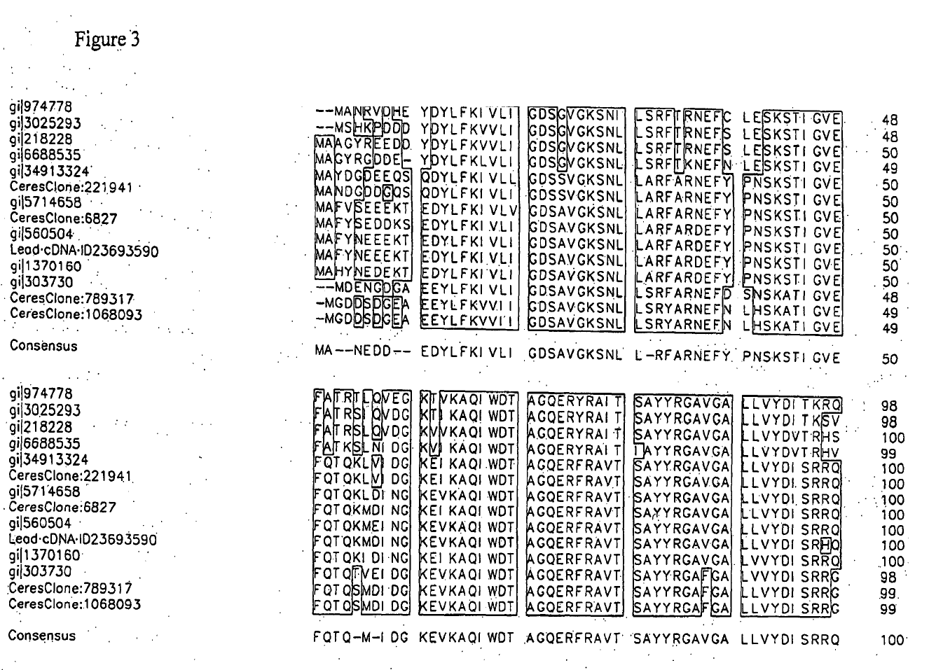

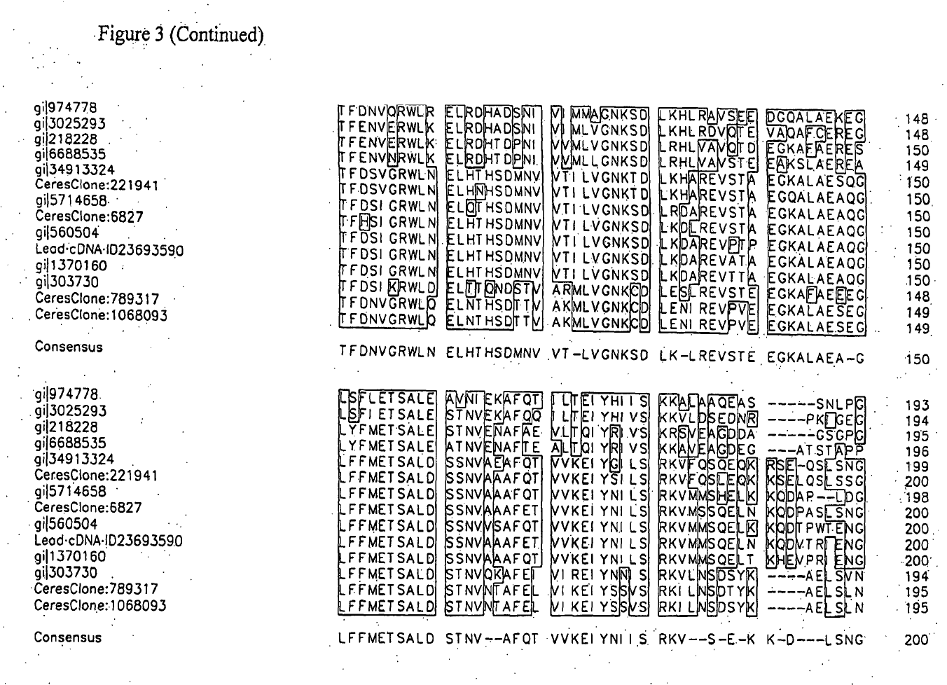

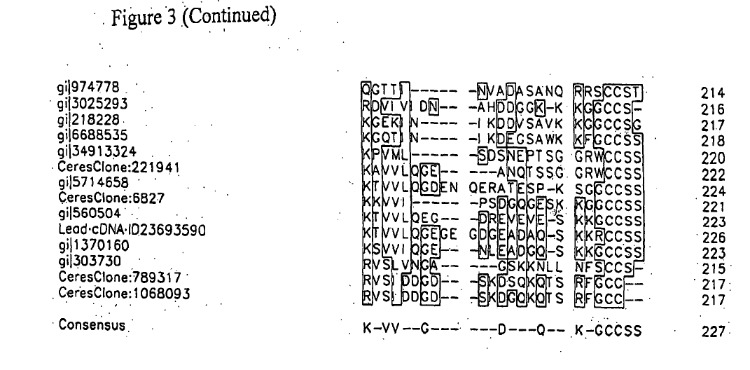

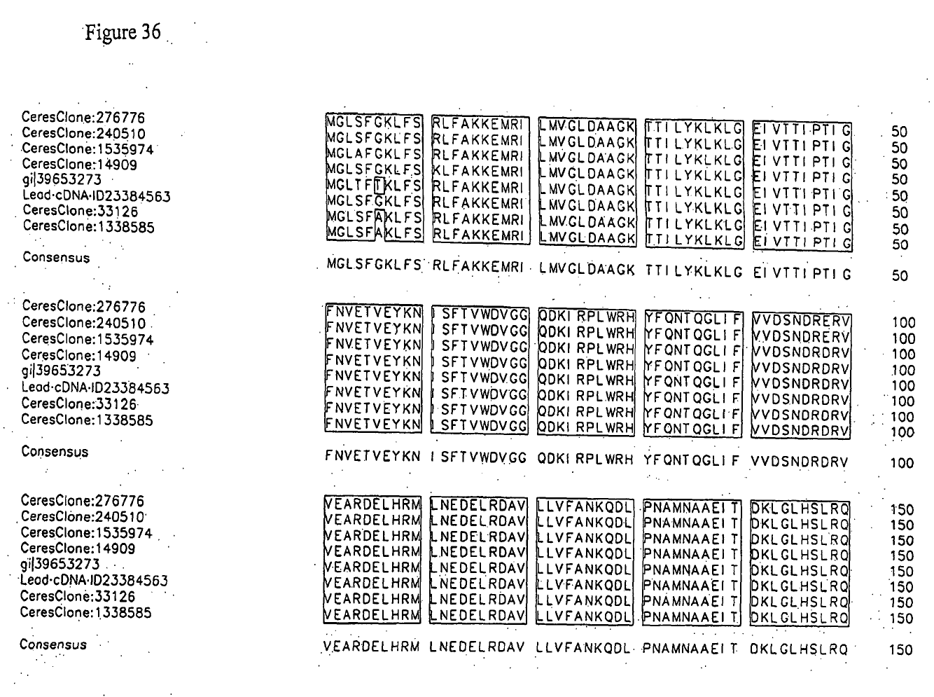

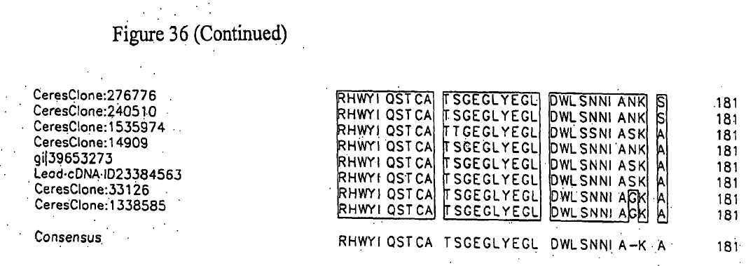

Amino acid sequences of homologs and/or orthologs of the polypeptide having the amino acid sequence set forth in SEQ ID NO:95 and SEQ ID NO:392 are provided in FIG. 3 and FIG. 36, respectively. Each of FIG. 3 and FIG. 36 also includes a consensus amino acid sequence determined by aligning homologous and/or orthologous amino acid sequences with the amino acid sequence set forth in SEQ ID NO:95 or SEQ ID NO:392, respectively.

For example, the alignment in FIG. 3 provides the amino acid sequences of cDNA ID 23693590 (SEQ ID NO:95), gi|1370160 (SEQ ID NO:96), gi|560504 (SEQ ID NO:97), CeresClone:6827 (SEQ ID NO:99), gi|5714658 (SEQ ID NO:100), gi|34913324 (SEQ ID NO:102), CeresClone:221941 (SEQ ID NO:103), gi|303730 (SEQ ID NO:104), gi|218228 (SEQ ID NO:105), CeresClone:789317 (SEQ ID NO:106), CeresClone:1068093 (SEQ ID NO:107), gi|974778 (SEQ ID NO:109), gi|3025293 (SEQ ID NO:110), and gi|6688535 (SEQ ID NO:111). Other homologs and/or orthologs of SEQ ID NO:95 include Public GI no. 541980 (SEQ ID NO:98), Public GI no. 5714660 (SEQ ID NO:101), and Public GI no. 53792703 (SEQ ID NO:108).

The alignment in FIG. 36 provides the amino acid sequences of cDNA ID 23384563 (SEQ ID NO:392) with homologous and/or orthologous amino acid sequences CeresClone:14909 (SEQ ID NO:393), CeresClone:33126 (SEQ ID NO:394), CeresClone:1338585 (SEQ ID NO:395), gi|39653273 (SEQ ID NO:396), CeresClone:276776 (SEQ ID NO:397), CeresClone:1535974 (SEQ ID NO:398), and CeresClone:240510 (SEQ ID NO:399).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:96-111, SEQ ID NOs:393-399, or the consensus sequences set forth in FIG. 3 or FIG. 36.

A regulatory protein can contain an RRM_{—}1 domain, described above, that is characteristic of an RNA binding polypeptide. SEQ ID NO:301, SEQ ID NO:345, SEQ ID NO:370, SEQ ID NO:382, SEQ ID NO:401, SEQ ID NO:411, SEQ ID NO:973, SEQ ID NO:1165, and SEQ ID NO:1178 set forth the amino acid sequences of DNA clones, identified herein as cDNA ID 23649144 (SEQ ID NO:300), cDNA ID 23460392 (SEQ ID NO:344), cDNA ID 23666854 (SEQ ID NO:369), cDNA ID 23698996 (SEQ ID NO:381), cDNA ID 23389848 (SEQ ID NO:400), cDNA ID 23384591 (SEQ ID NO:410), cDNA ID 23380615 (SEQ ID NO:972), cDNA ID 23375896 (SEQ ID NO:1164), and cDNA ID 23369842 (SEQ ID NO:1177), respectively, each of which is predicted to encode an RRM_{—}1-containing polypeptide.

A regulatory protein can comprise the amino acid sequence set forth in SEQ ID NO:301, SEQ ID NO:345, SEQ ID NO:370, SEQ ID NO:382, SEQ ID NO:401, SEQ ID NO:411, SEQ ID NO:973, SEQ ID NO:1165, or SEQ ID NO:1178. Alternatively, a regulatory protein can be a homolog, ortholog, or variant of the polypeptide having the amino acid sequence set forth in SEQ ID NO:301, SEQ ID NO:345, SEQ ID NO:370, SEQ ID NO:382, SEQ ID NO:401, SEQ ID NO:411, SEQ ID NO:973, SEQ ID NO:1165, or SEQ ID NO:1178. For example, a regulatory protein can have an amino acid sequence with at least 35% sequence identity, e.g., 35%, 40%, 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:301, SEQ ID NO:345, SEQ ID NO:370, SEQ ID NO:382, SEQ ID NO:401, SEQ ID NO:411, SEQ ID NO:973, SEQ ID NO:1165, or SEQ ID NO:1178.

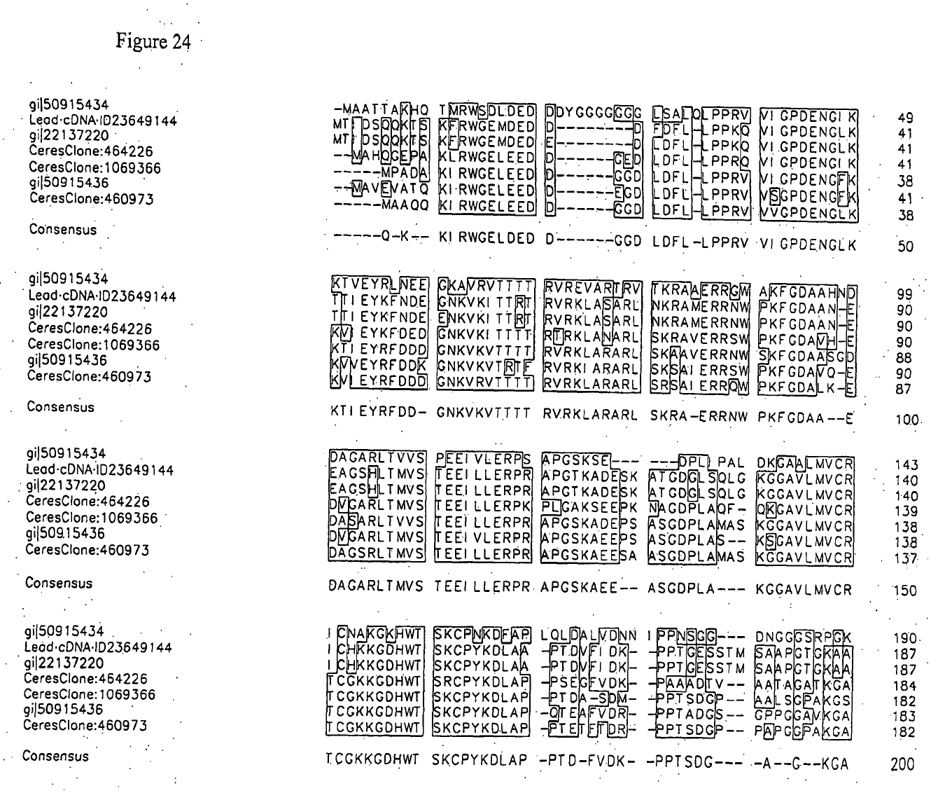

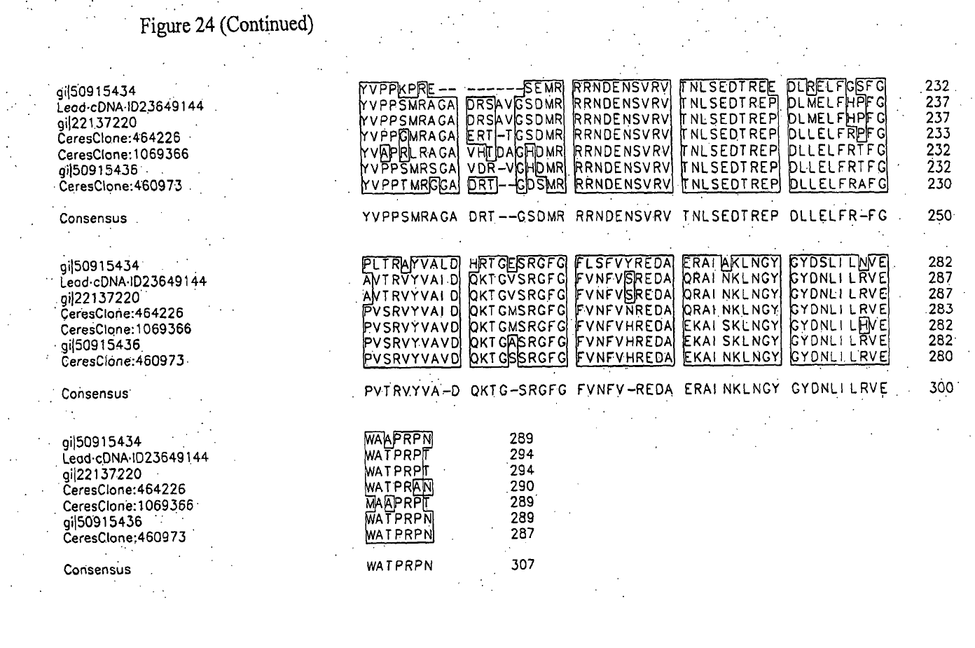

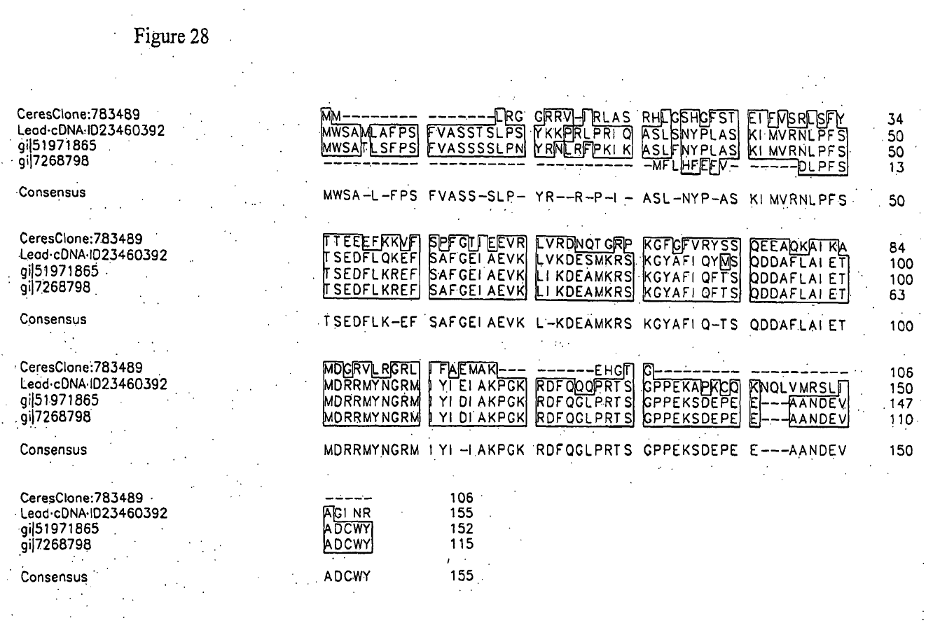

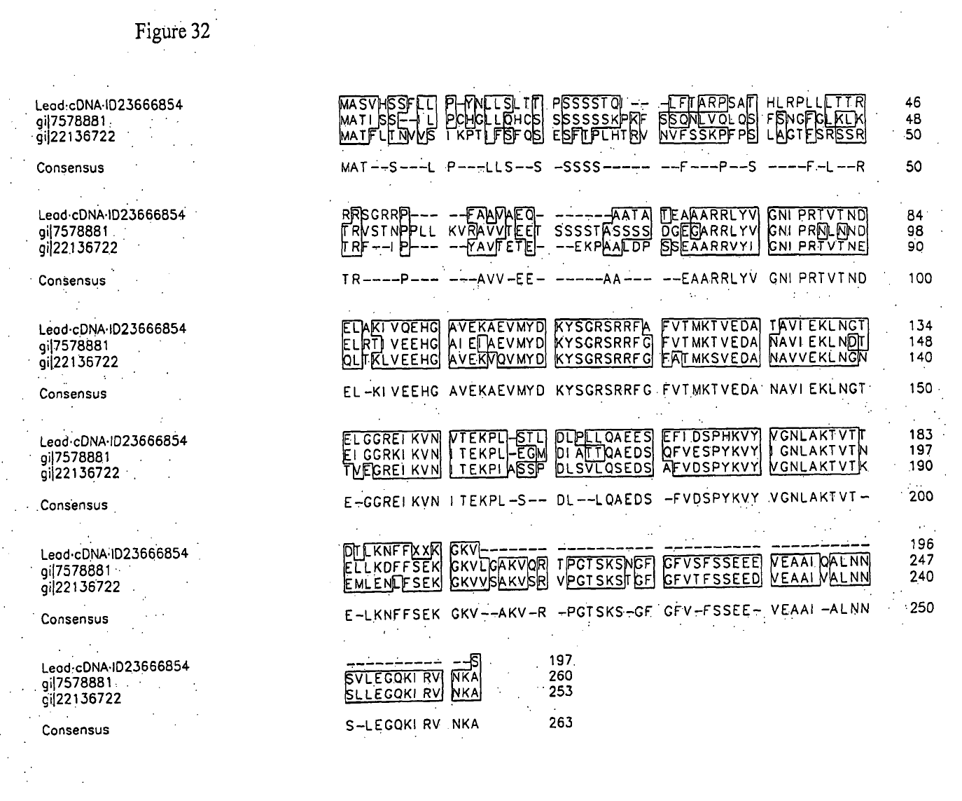

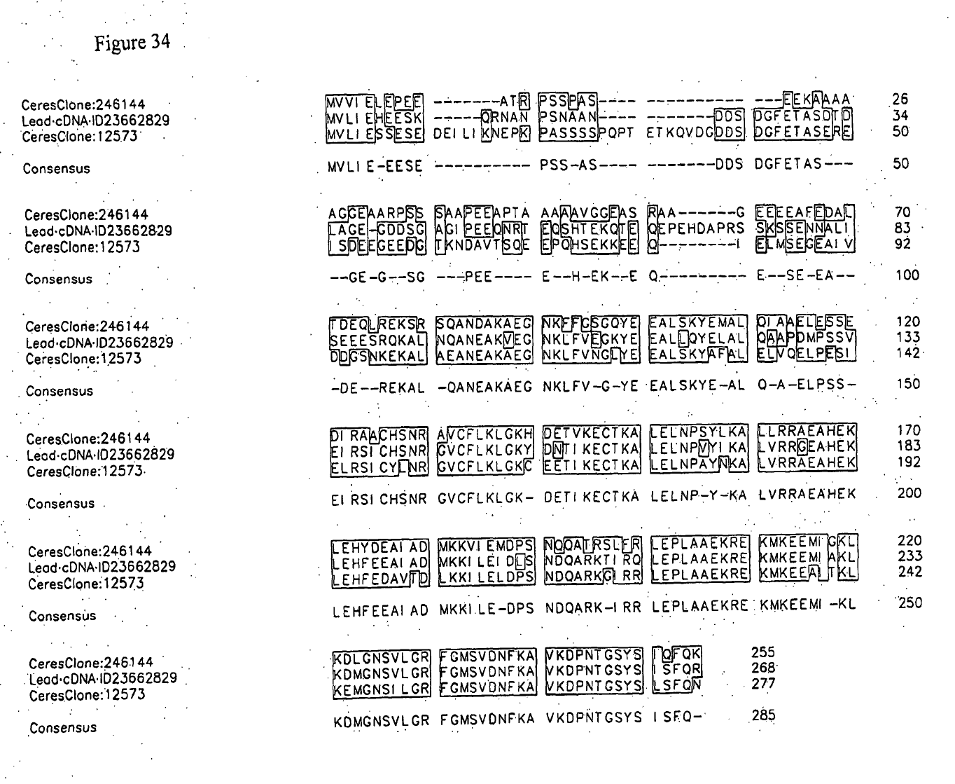

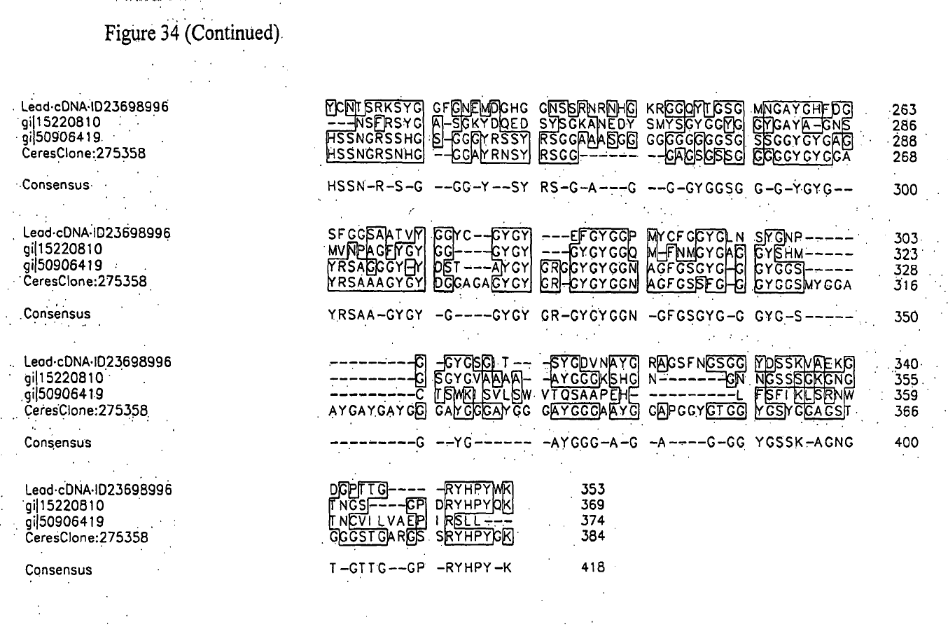

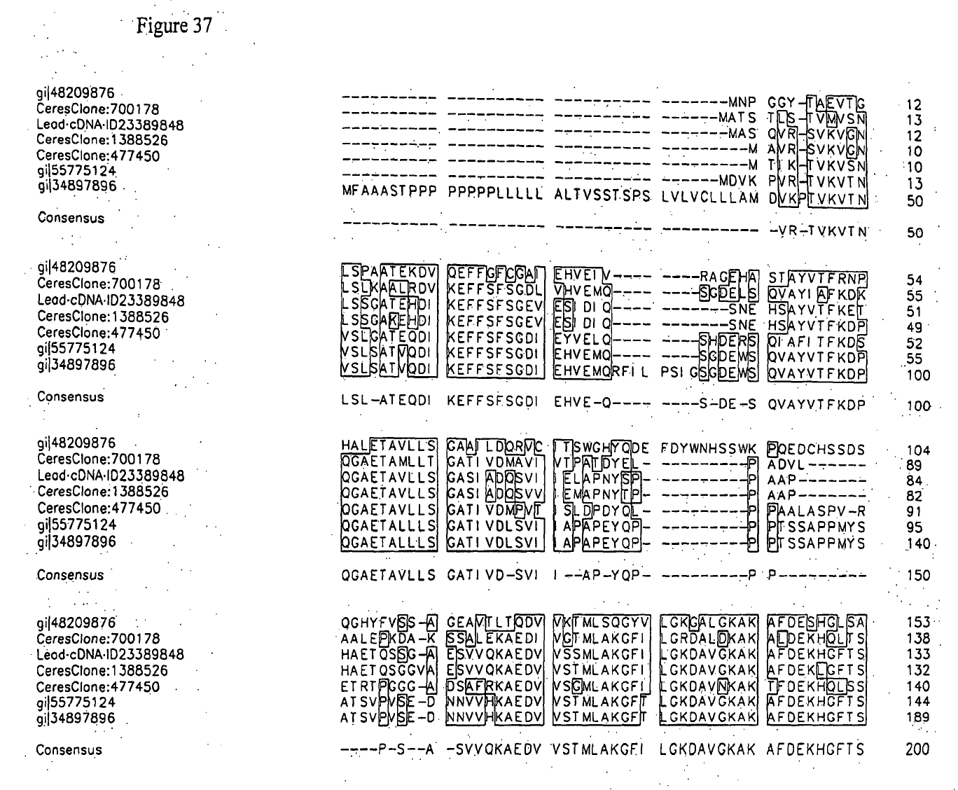

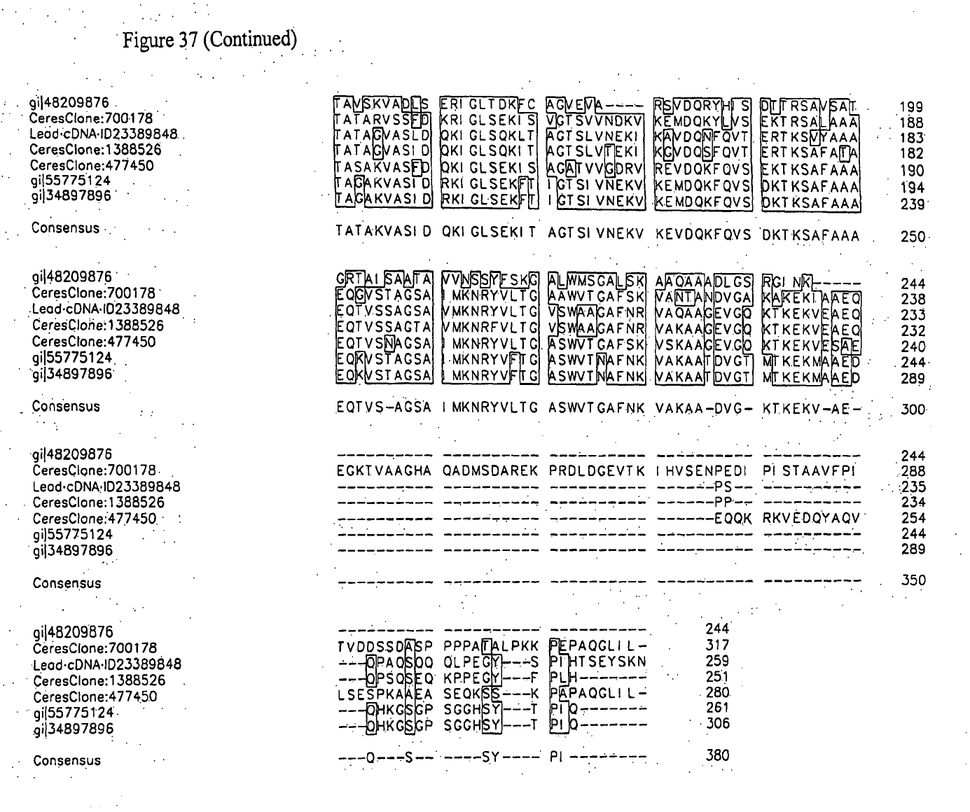

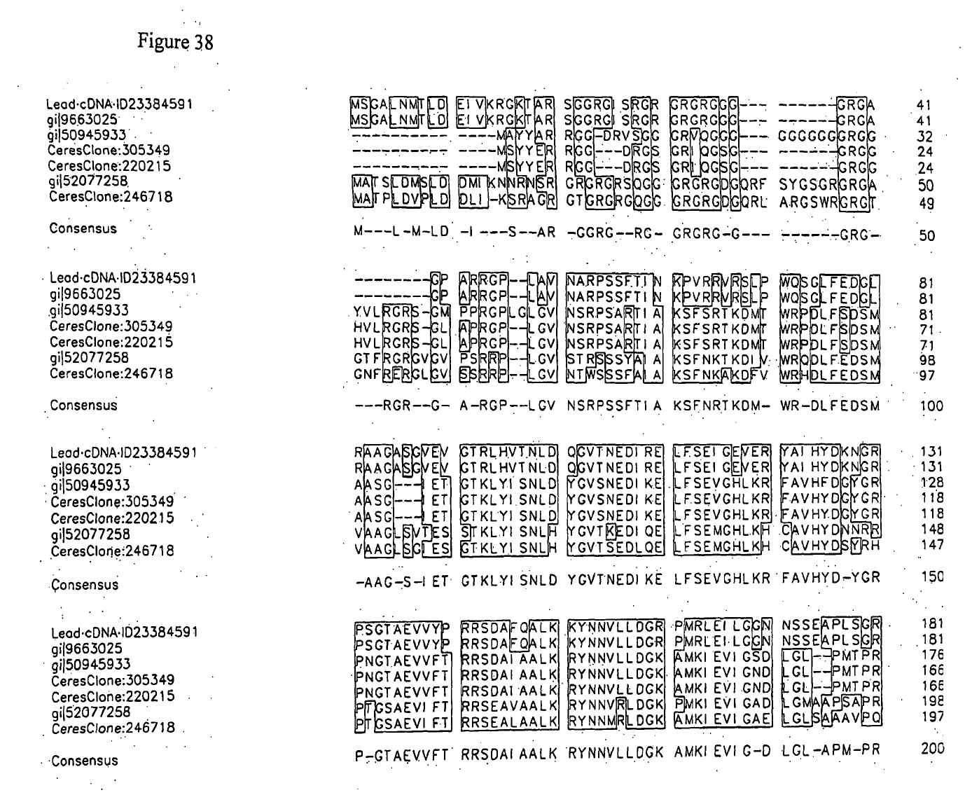

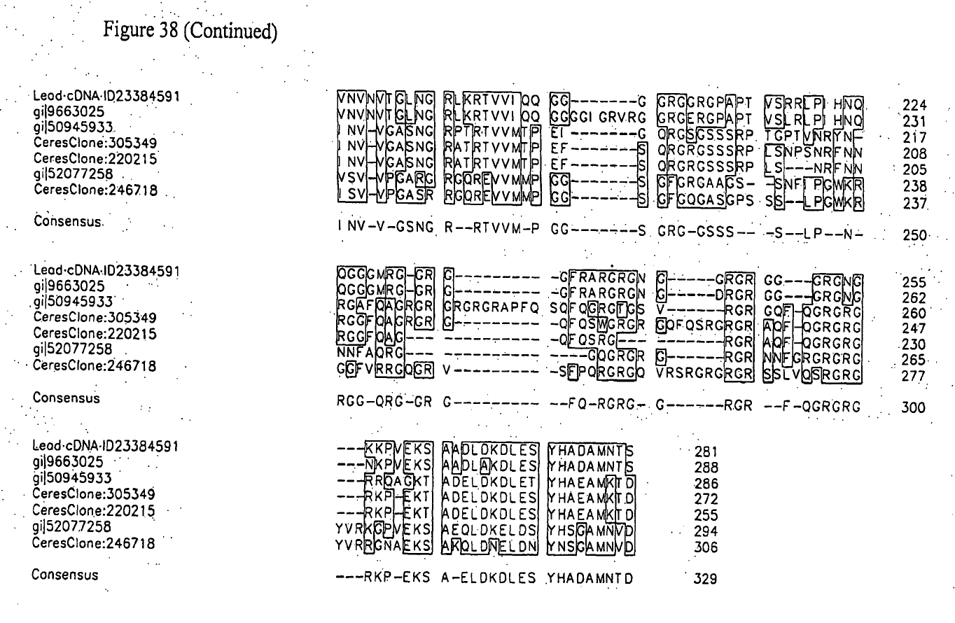

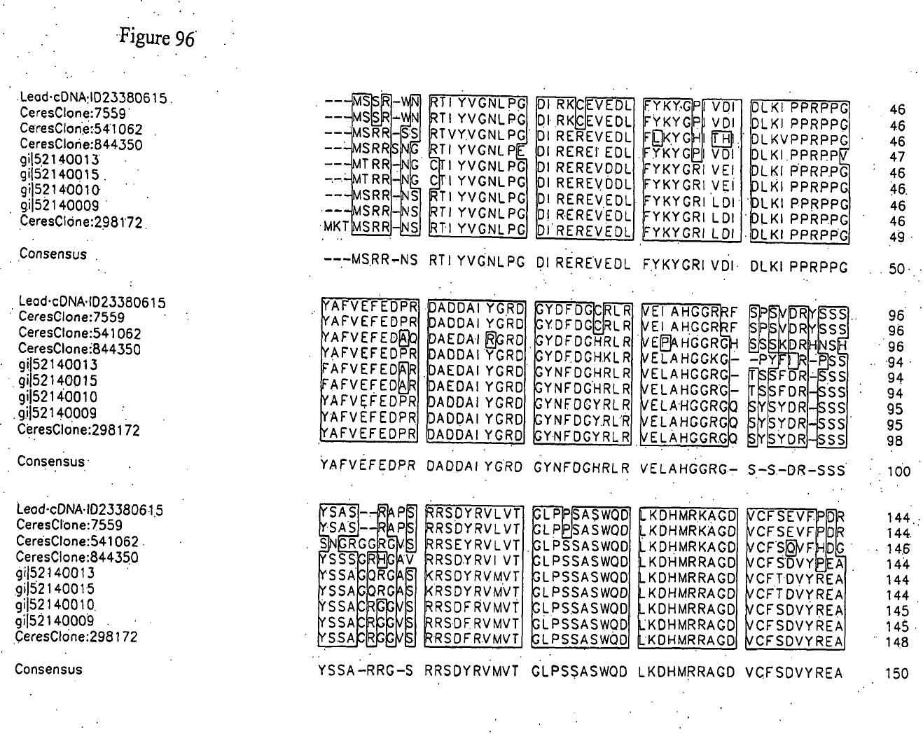

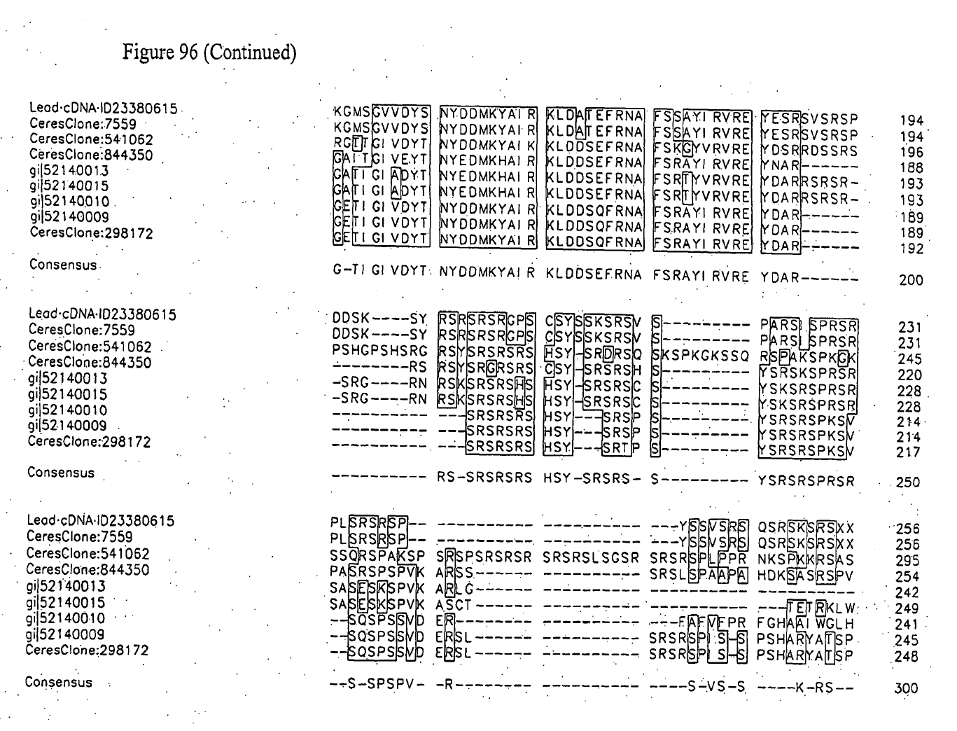

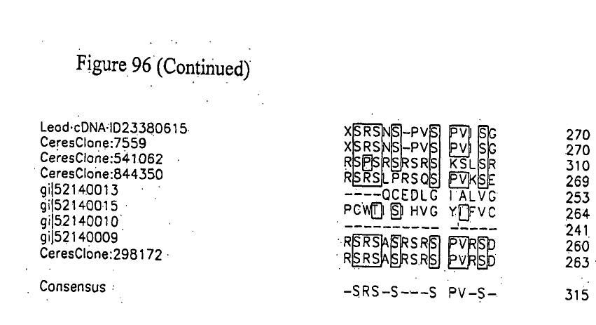

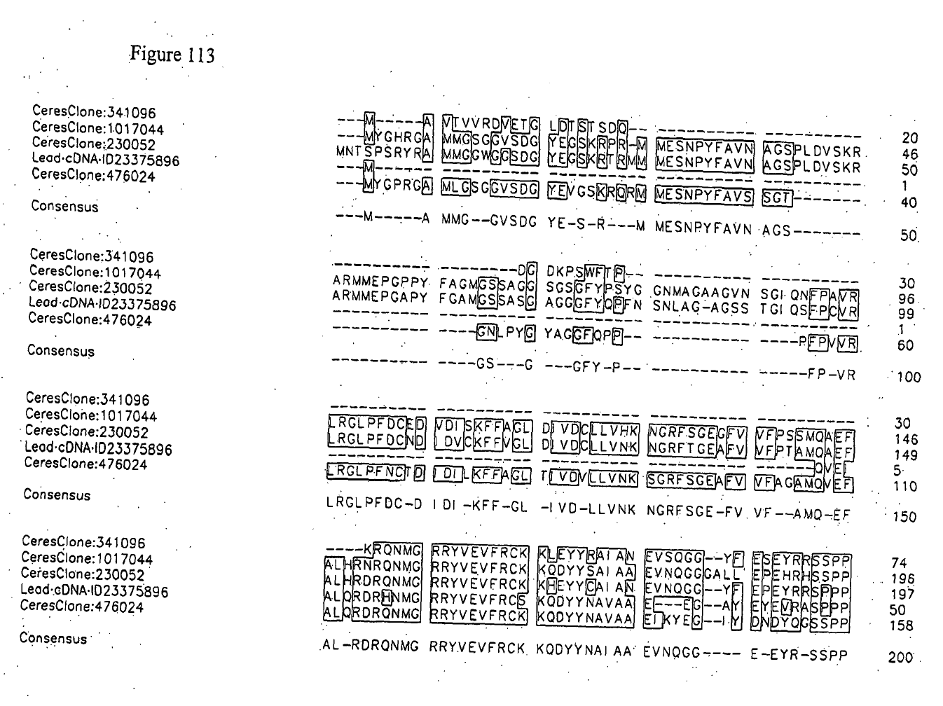

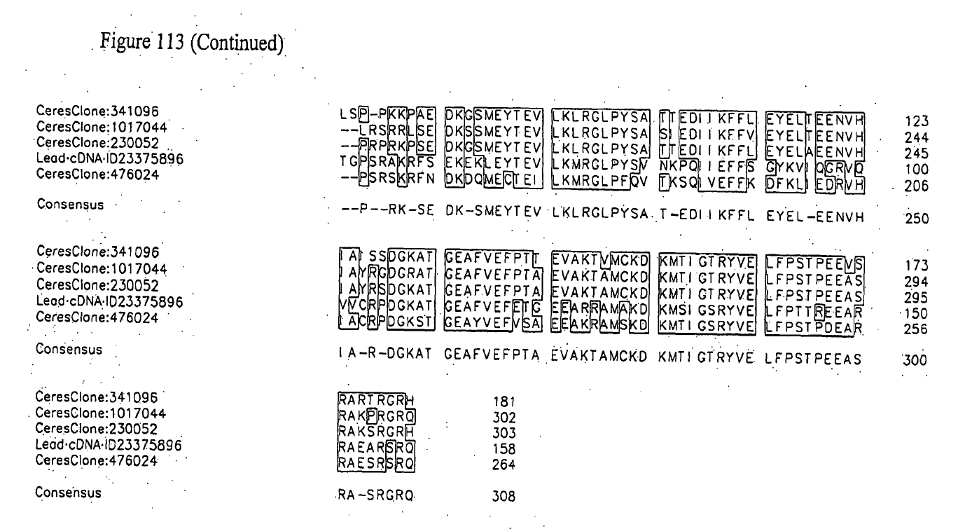

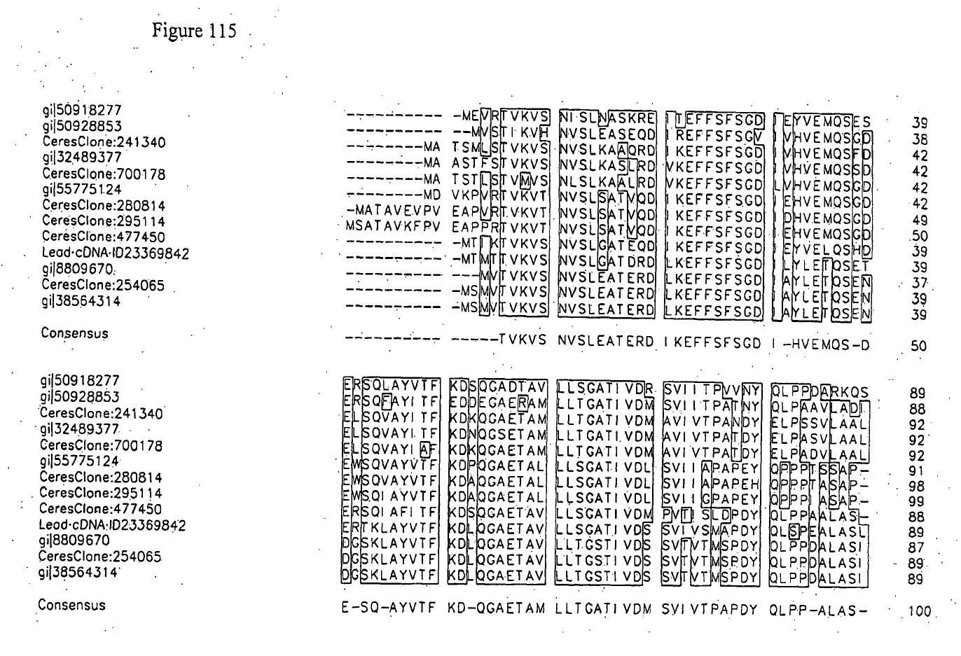

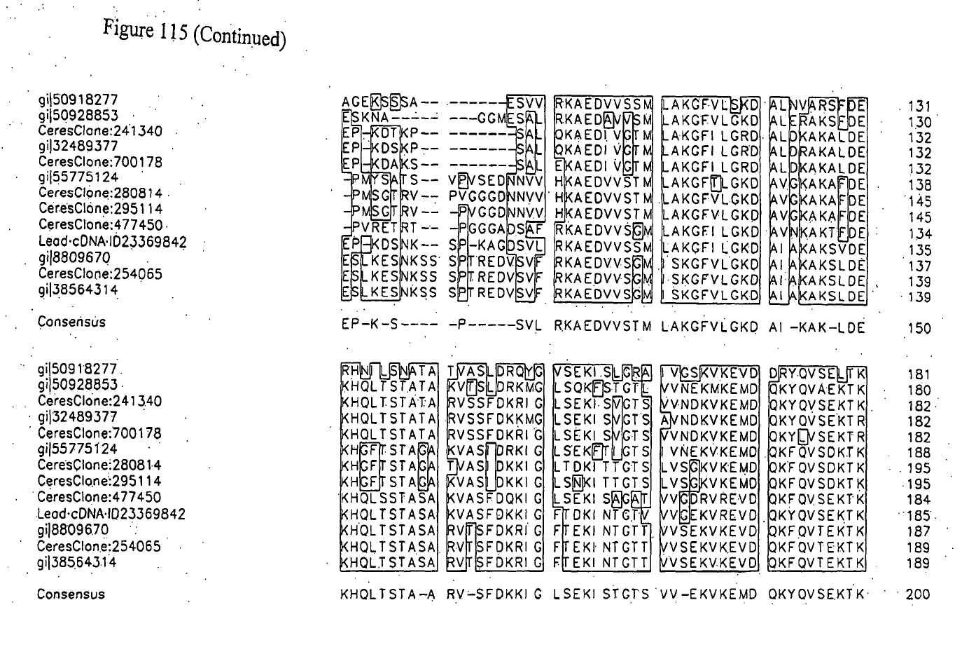

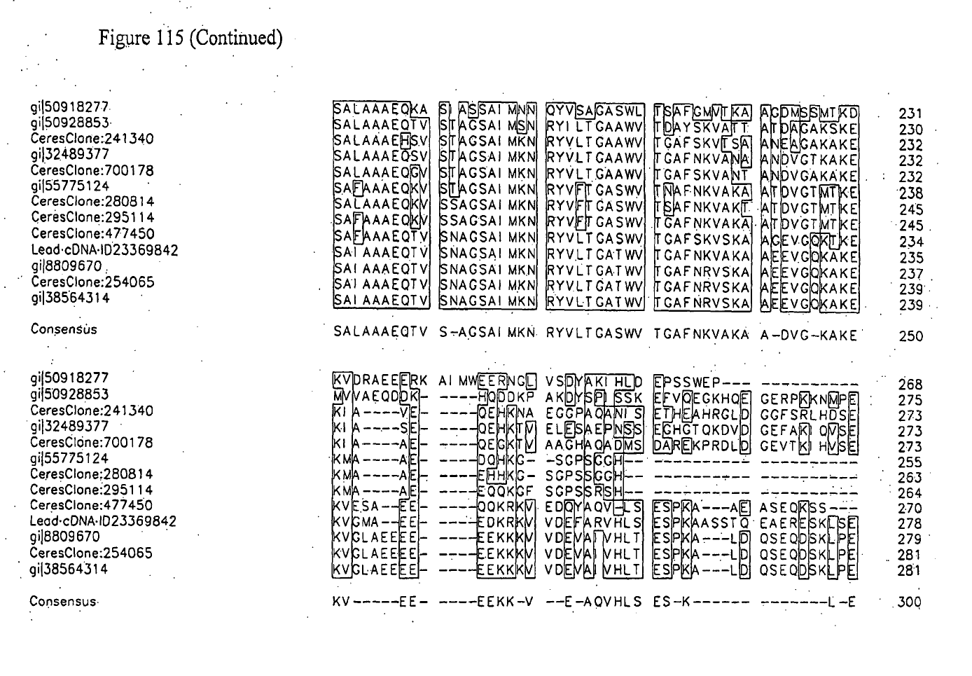

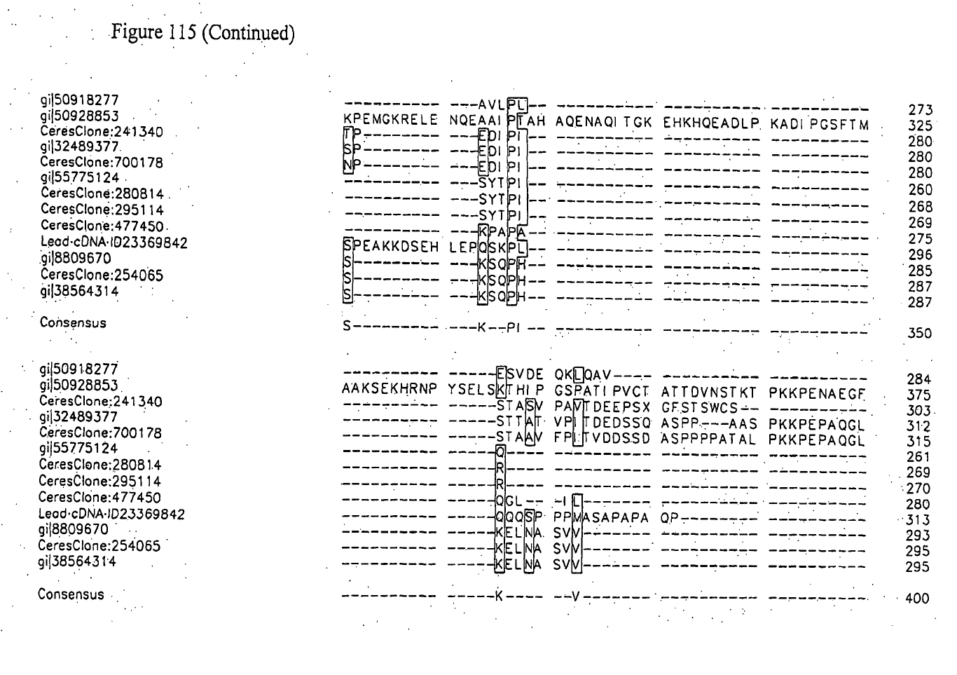

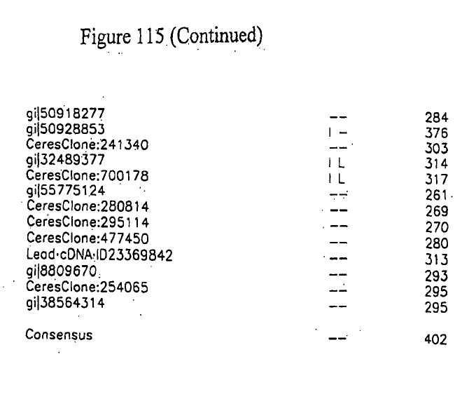

Amino acid sequences of homologs and/or orthologs of the polypeptide having the amino acid sequence set forth in SEQ ID NO:301, SEQ ID NO:345, SEQ ID NO:370, SEQ ID NO:382, SEQ ID NO:401, SEQ ID NO:411, SEQ ID NO:973, SEQ ID NO:1165, and SEQ ID NO:1178 are provided in FIG. 24, FIG. 28, FIG. 32, FIG. 34, FIG. 37, FIG. 38, FIG. 96, FIG. 113, and FIG. 115, respectively. Each of FIG. 24, FIG. 28, FIG. 32, FIG. 34, FIG. 37, FIG. 38, FIG. 96, FIG. 113, and FIG. 115 also includes a consensus amino acid sequence determined by aligning homologous and/or orthologous amino acid sequences with the amino acid sequence set forth in SEQ ID NO:301, SEQ ID NO:345, SEQ ID NO:370, SEQ ID NO:382, SEQ ID NO:401, SEQ ID NO:411, SEQ ID NO:973, SEQ ID NO:1165, or SEQ ID NO:1178, respectively.

For example, the alignment in FIG. 24 provides the amino acid sequences of cDNA ID 23649144 (SEQ ID NO:301), gi|22137220 (SEQ ID NO:302), CeresClone:460973 (SEQ ID NO:303), CeresClone:464226 (SEQ ID NO:304), gi|50915436 (SEQ ID NO:305), CeresClone:1069366 (SEQ ID NO:306), and gi|50915434 (SEQ ID NO:307).

The alignment in FIG. 28 provides the amino acid sequences of cDNA ID 23460392 (SEQ ID NO:345), gi|51971865 (SEQ ID NO:346), gi|7268798 (SEQ ID NO:347), and CeresClone:783489 (SEQ ID NO:348).

The alignment in FIG. 32 provides the amino acid sequences of cDNA ID 23666854 (SEQ ID NO:370), gi|22136722 (SEQ ID NO:373) and gi|7578881 (SEQ ID NO:374). Other homologs and/or orthologs of SEQ ID NO:370 include Ceres CLONE ID no. 480900 (SEQ ID NO:371) and Ceres CLONE ID no. 652078 (SEQ ID NO:372).

The alignment in FIG. 34 provides the amino acid sequences of cDNA ID 23698996 (SEQ ID NO:382), gi|50906419 (SEQ ID NO:383), gi|15220810 (SEQ ID NO:384), and CeresClone:275358 (SEQ ID NO:385).

The alignment in FIG. 37 provides the amino acid sequences of cDNA ID 23389848 (SEQ ID NO:401), CeresClone:1388526 (SEQ ID NO:402), gi|55775124 (SEQ ID NO:403), CeresClone:477450 (SEQ ID NO:404), gi|34897896 (SEQ ID NO:405), CeresClone:700178 (SEQ ID NO:406), and gi|48209876 (SEQ ID NO:407). Other homologs and/or orthologs of SEQ ID NO:401 include Public GI no. 48209951 (SEQ ID NO:408) and Public GI no. 48057564 (SEQ ID NO:409).

The alignment in FIG. 38 provides the amino acid sequences of cDNA ID 23384591 (SEQ ID NO:411), gi|9663025 (SEQ ID NO:412), CeresClone:305349 (SEQ ID NO:413), CeresClone:220215 (SEQ ID NO:414), gi|50945933 (SEQ ID NO:415), gi|52077258 (SEQ ID NO:416), and CeresClone:246718 (SEQ ID NO:417).

The alignment in FIG. 96 provides the amino acid sequences of cDNA ID 23380615 (SEQ ID NO:973), CeresClone:7559 (SEQ ID NO:974), gi|52140010 (SEQ ID NO:975), CeresClone:844350 (SEQ ID NO:976), gi|52140009 (SEQ ID NO:977), CeresClone:298172 (SEQ ID NO:978), gi|52140013 (SEQ ID NO:979), CeresClone:541062 (SEQ ID NO:980), and gi|52140015 (SEQ ID NO:981).

The alignment in FIG. 113 provides the amino acid sequences of cDNA ID 23375896 (SEQ ID NO:1165), CeresClone:476024 (SEQ ID NO:1166), CeresClone:1017044 (SEQ ID NO:1167), CeresClone:230052 (SEQ ID NO:1168), and CeresClone:341096 (SEQ ID NO:1169).

The alignment in FIG. 115 provides the amino acid sequences of cDNA ID 23369842 (SEQ ID NO:1178), gi|8809670 (SEQ ID NO:1179), CeresClone:254065 (SEQ ID NO:1180), gi|38564314 (SEQ ID NO:1181), CeresClone:477450 (SEQ ID NO:1182), CeresClone:280814 (SEQ ID NO:1183), gi|55775124 (SEQ ID NO:1184), CeresClone:295114 (SEQ ID NO:1185), CeresClone:241340 (SEQ ID NO:1186), gi|32489377 (SEQ ID NO:1187), CeresClone:700178 (SEQ ID NO:1188), gi|50928853 (SEQ ID NO:1189), and gi|50918277 (SEQ ID NO:1190).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:302-307, SEQ ID NOs:346-348, SEQ ID NOs:371-374, SEQ ID NOs:383-385, SEQ ID NOs:402-409, SEQ ID NOs:412-417, SEQ ID NOs:974-981, SEQ ID NOs:1166-1169, SEQ ID NOs:1179-1190, or the consensus sequences set forth in FIG. 24, FIG. 28, FIG. 32, FIG. 34, FIG. 37, FIG. 38, FIG. 96, FIG. 113, or FIG. 115.

A regulatory protein can contain a GRP domain characteristic of a polypeptide belonging to the glycine-rich protein family. This family of proteins includes several glycine-rich proteins as well as two nodulins 16 and 24. The family also contains proteins that are induced in response to various stresses. Some of the proteins that have a glycine-rich domain (i.e., GRPs) are capable of binding to RNA, potentially affecting the stability and translatability of bound RNAs. SEQ ID NO:931, SEQ ID NO:1127, SEQ ID NO:1279, and SEQ ID NO:1342 set forth the amino acid sequences of DNA clones, identified herein as cDNA ID 23389966 (SEQ ID NO:930), cDNA ID 23380898 (SEQ ID NO:1126), cDNA ID 23390282 (SEQ ID NO:1278), and cDNA ID 23449316 (SEQ ID NO:1341), respectively, that are predicted to encode glycine-rich proteins.

A regulatory protein can comprise the amino acid sequence set forth in SEQ ID NO:931, SEQ ID NO:1127, SEQ ID NO:1279, or SEQ ID NO:1342. Alternatively, a regulatory protein can be a homolog, ortholog, or variant of the polypeptide having the amino acid sequence set forth in SEQ ID NO:931, SEQ ID NO:1127, SEQ ID NO:1279, or SEQ ID NO:1342. For example, a regulatory protein can have an amino acid sequence with at least 35% sequence identity, e.g., 35%, 40%, 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:931, SEQ ID NO:1127, SEQ ID NO:1279, or SEQ ID NO:1342.

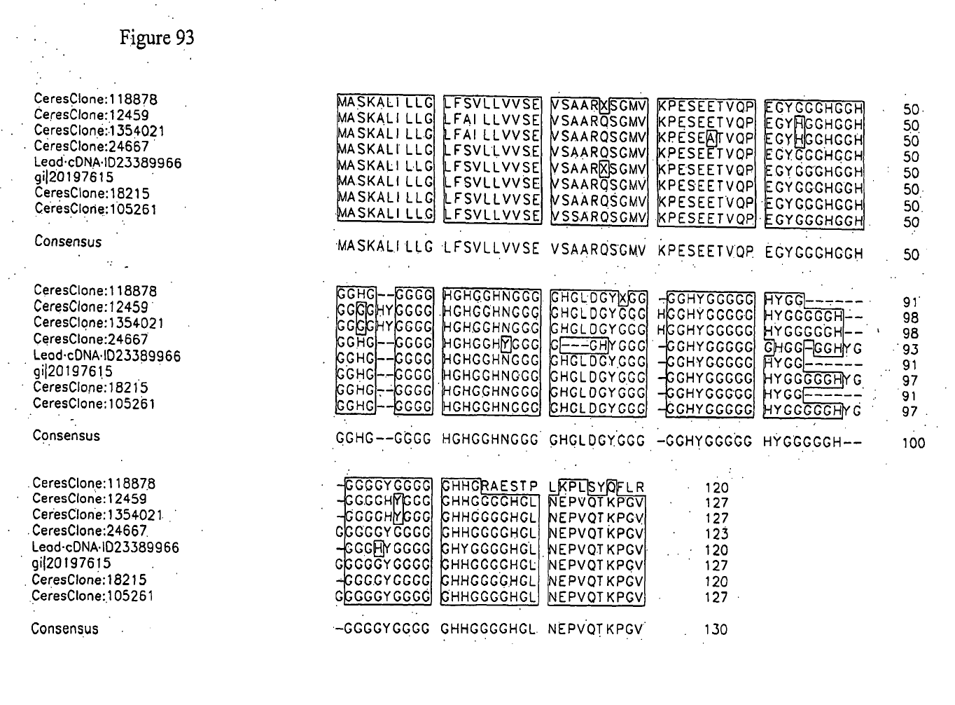

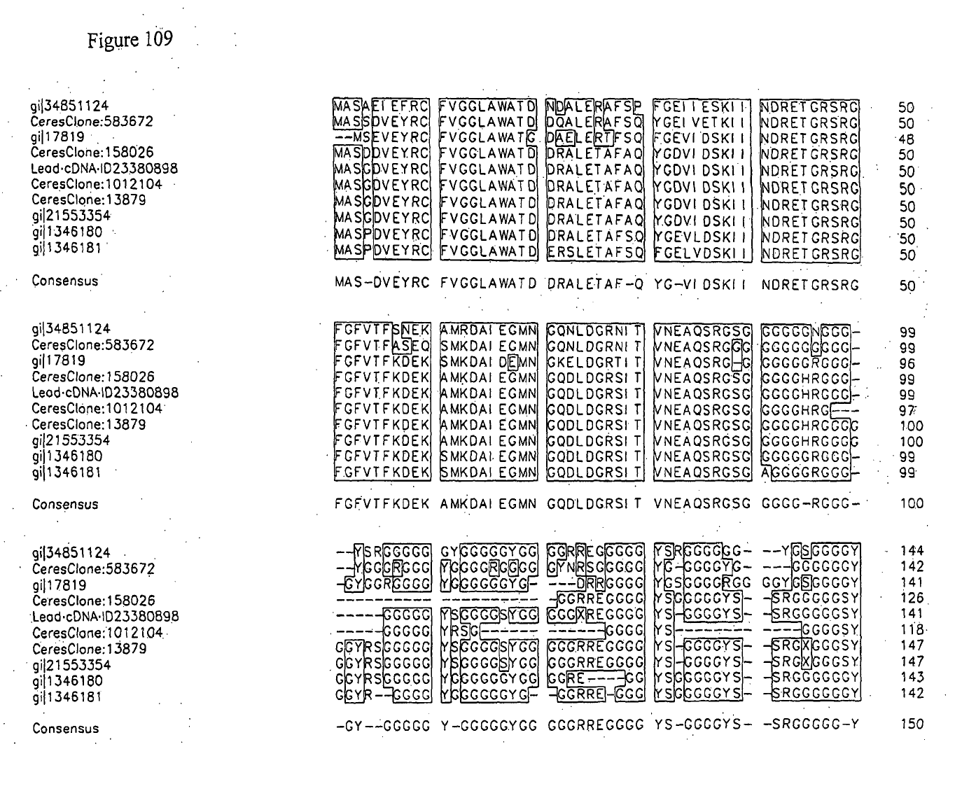

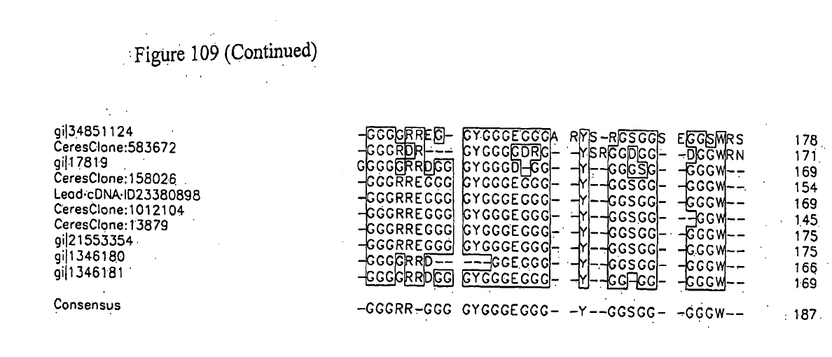

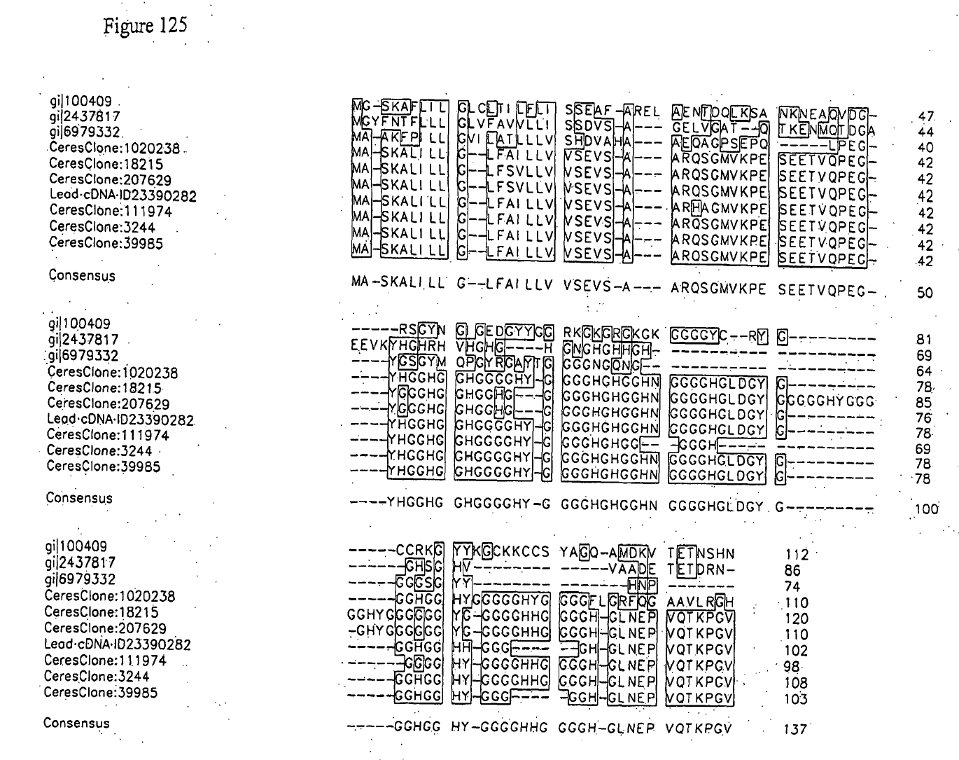

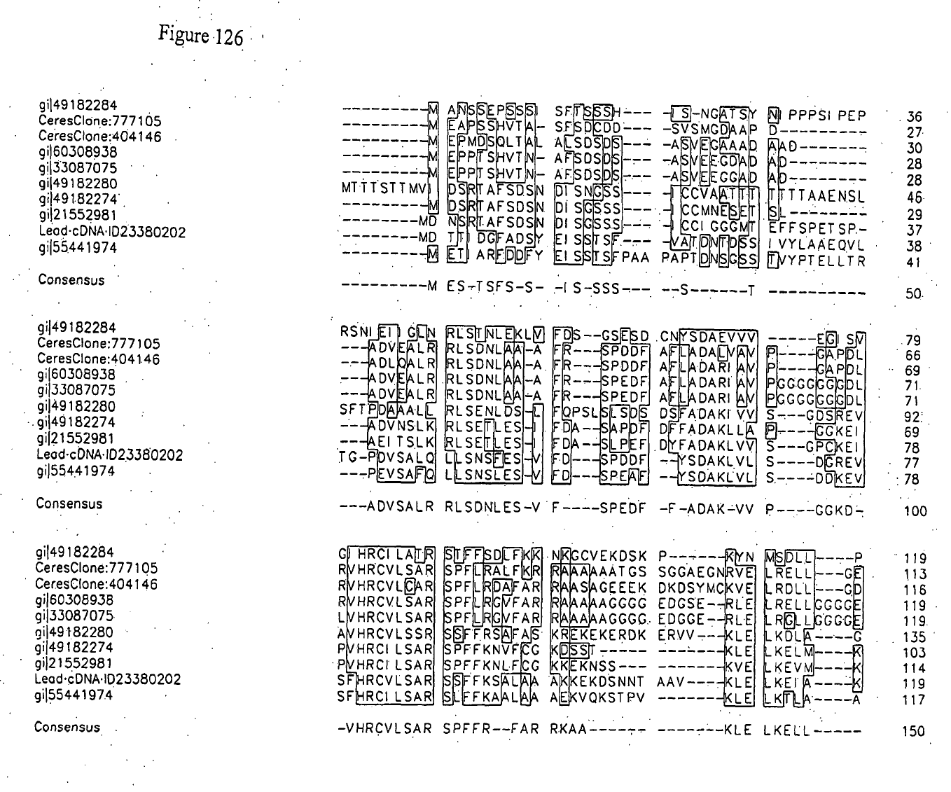

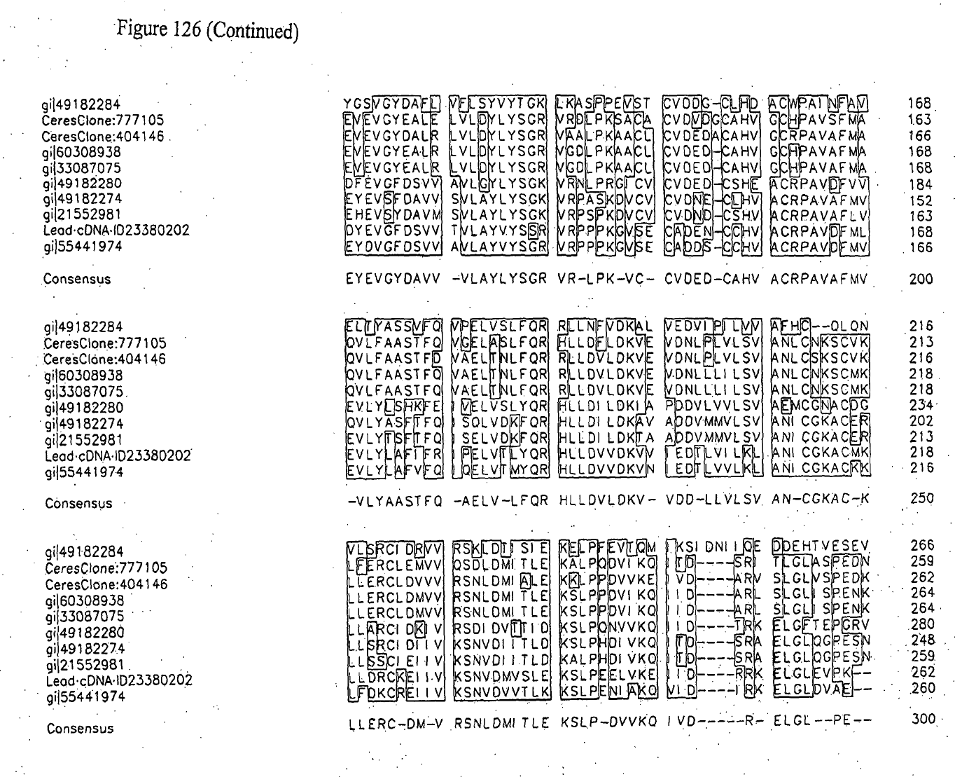

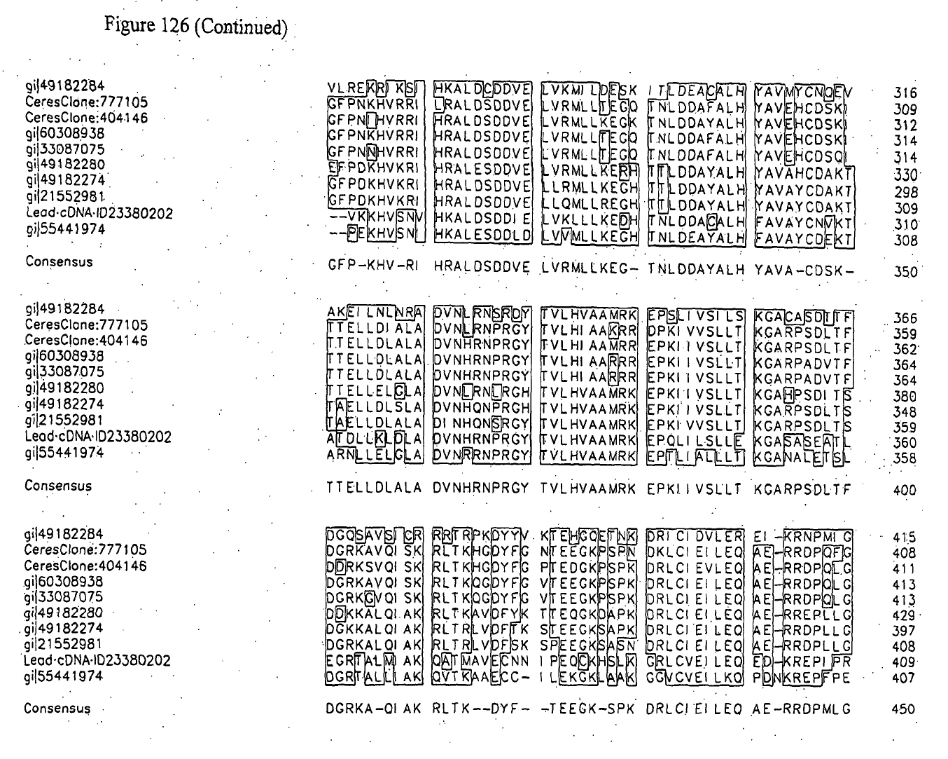

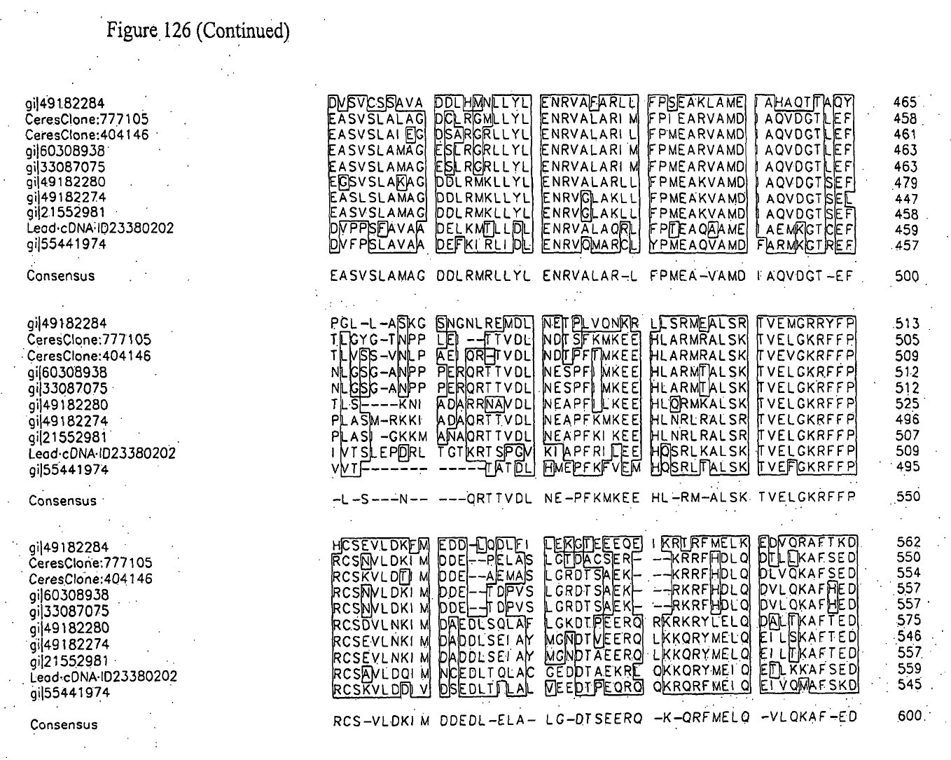

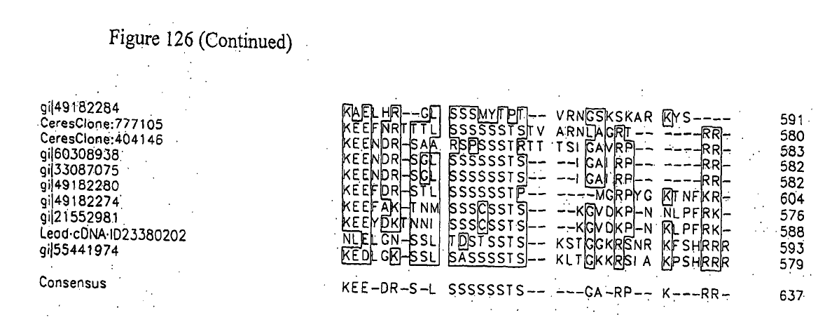

Amino acid sequences of homologs and/or orthologs of the polypeptide having the amino acid sequence set forth in SEQ ID NO:931, SEQ ID NO:1127, and SEQ ID NO:1279 are provided in FIG. 93, FIG. 109, and FIG. 125, respectively. Each of FIG. 93, FIG. 109, and FIG. 125 also includes a consensus amino acid sequence determined by aligning homologous and/or orthologous amino acid sequences with the amino acid sequence set forth in SEQ ID NO:931, SEQ ID NO:1127, or SEQ ID NO:1279, respectively.

For example, the alignment in FIG. 93 provides the amino acid sequences of cDNA ID 23389966 (SEQ ID NO:931), gi|20197615 (SEQ ID NO:932), CeresClone:18215 (SEQ ID NO:933), CeresClone:105261 (SEQ ID NO:935), CeresClone:24667 (SEQ ID NO:938), CeresClone:118878 (SEQ ID NO:940), CeresClone:12459 (SEQ ID NO:941), and CeresClone:1354021 (SEQ ID NO:942). Other homologs and/or orthologs of SEQ ID NO:931 include Public GI no. 21536606 (SEQ ID NO:934), Ceres CLONE ID no. 23214 (SEQ ID NO:936), Ceres CLONE ID no. 207629 (SEQ ID NO:937), Ceres CLONE ID no. 1006473 (SEQ ID NO:939), Public GI no. 30017217 (SEQ ID NO:943), and Ceres CLONE ID no. 109026 (SEQ ID NO:944).

The alignment in FIG. 109 provides the amino acid sequences of cDNA ID 23380898 (SEQ ID NO:1127), CeresClone:13879 (SEQ ID NO:1128), gi|21553354 (SEQ ID NO:1129), CeresClone:158026 (SEQ ID NO:1130), CeresClone:1012104 (SEQ ID NO:1131), gi|1346180 (SEQ ID NO:1132), gi|1346181 (SEQ ID NO:1133), gi|17819 (SEQ ID NO:1134), gi|34851124 (SEQ ID NO:1135), and CeresClone:583672 (SEQ ID NO:1136).

The alignment in FIG. 125 provides the amino acid sequences of cDNA ID 23390282 (SEQ ID NO:1279), CeresClone:3244 (SEQ ID NO:1280), CeresClone:39985 (SEQ ID NO:1282), CeresClone:1020238 (SEQ ID NO:1287), CeresClone:18215 (SEQ ID NO:1288), CeresClone:111974 (SEQ ID NO:1290), CeresClone:207629 (SEQ ID NO:1291), gi|6979332 (SEQ ID NO:1293), gi|2437817 (SEQ ID NO:1294), and gi|100409 (SEQ ID NO:1295). Other homologs and/or orthologs of SEQ ID NO:1279 include Ceres CLONE ID no. 12459 (SEQ ID NO:1281), Ceres CLONE ID no. 1354021 (SEQ ID NO:1283), Public GI no. 30017217 (SEQ ID NO:1284), Ceres CLONE ID no. 114551 (SEQ ID NO:1285), Ceres CLONE ID no. 102088 (SEQ ID NO:1286), Ceres CLONE ID no. 23214 (SEQ ID NO:1289), and Ceres CLONE ID no. 3929 (SEQ ID NO:1292).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:932-944, SEQ ID NOs:1128-1136, SEQ ID NOs:1280-1295, or the consensus sequences set forth in FIG. 93, FIG. 109, or FIG. 125.

A regulatory protein can contain one or more domains characteristic of a helicase polypeptide. For example, a regulatory protein can contain a Helicase_C domain and a DEAD domain characteristic of a DEAD/DEAH box helicase polypeptide. Members of the DEAD/DEAH box helicase polypeptide family include the DEAD and DEAH box helicases. Helicases are involved in unwinding nucleic acids. The DEAD box helicases are involved in various aspects of RNA metabolism, including nuclear transcription, pre mRNA splicing, ribosome biogenesis, nucleocytoplasmic transport, translation, RNA decay and organellar gene expression. The Helicase_C, or helicase conserved C-terminal, domain is found in a wide variety of helicases and related polypeptides. The Helicase_C domain may be an integral part of the helicase rather than an autonomously folding unit. SEQ ID NO:173, SEQ ID NO:711, and SEQ ID NO:1001 set forth the amino acid sequences of DNA clones, identified herein as cDNA ID 13653045 (SEQ ID NO:172), cDNA ID 23363175 (SEQ ID NO:710), and cDNA ID 23359888 (SEQ ID NO:1000), respectively, each of which is predicted to encode a polypeptide containing a DEAD domain and a Helicase_C domain.

A regulatory protein can comprise the amino acid sequence set forth in SEQ ID NO:173, SEQ ID NO:711, or SEQ ID NO:1001. Alternatively, a regulatory protein can be a homolog, ortholog, or variant of the polypeptide having the amino acid sequence set forth in SEQ ID NO:173, SEQ ID NO:711, or SEQ ID NO:1001. For example, a regulatory protein can have an amino acid sequence with at least 30% sequence identity, e.g., 30%, 35%, 40%, 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:173, SEQ ID NO:711, or SEQ ID NO:1001.

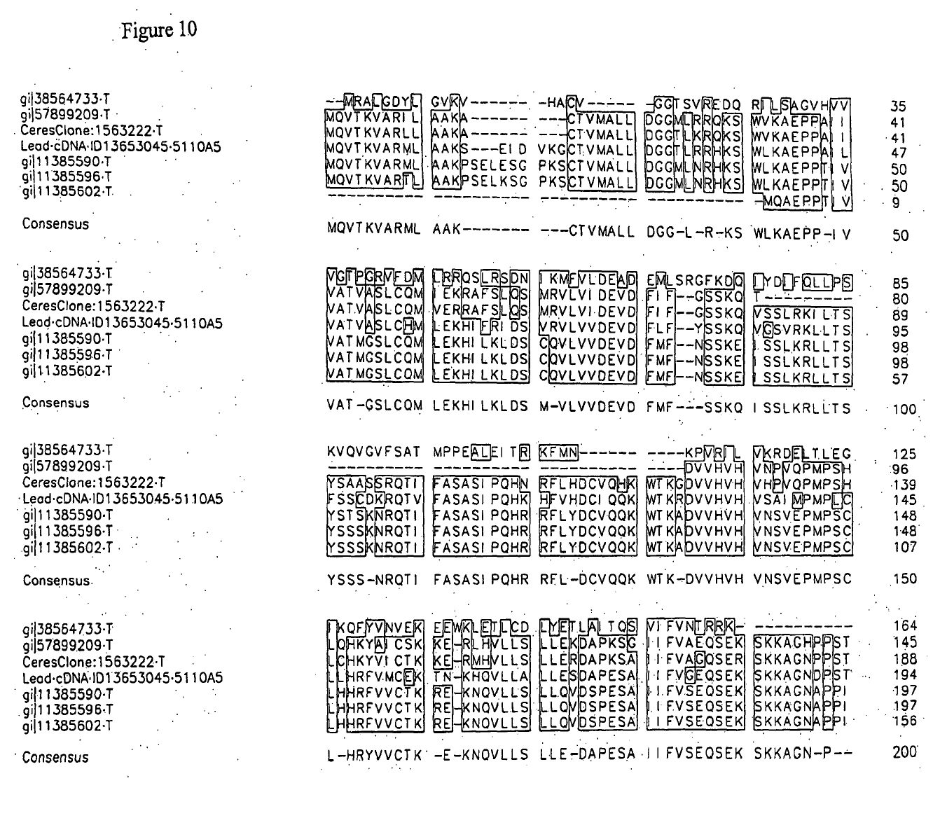

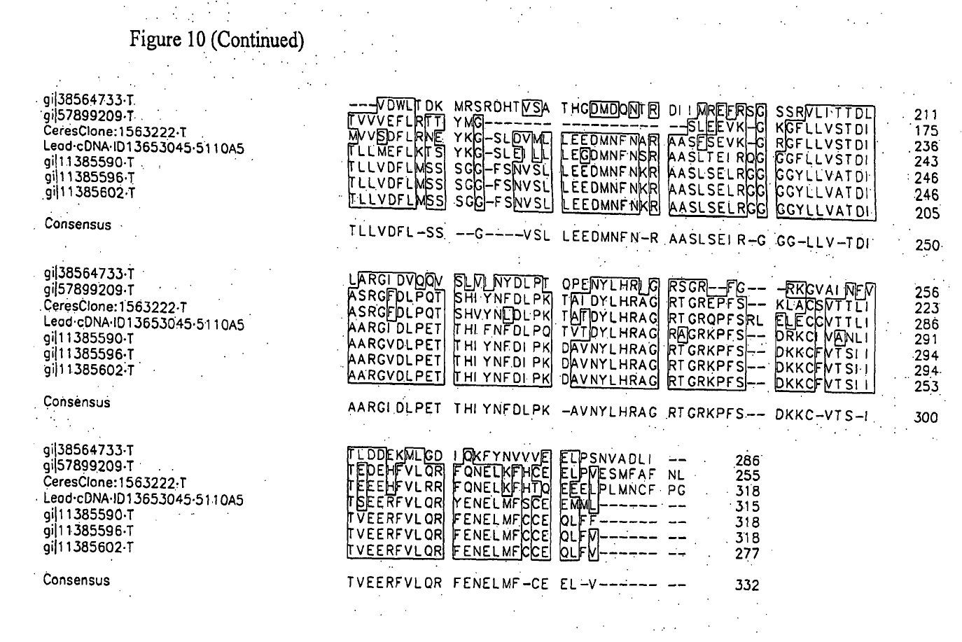

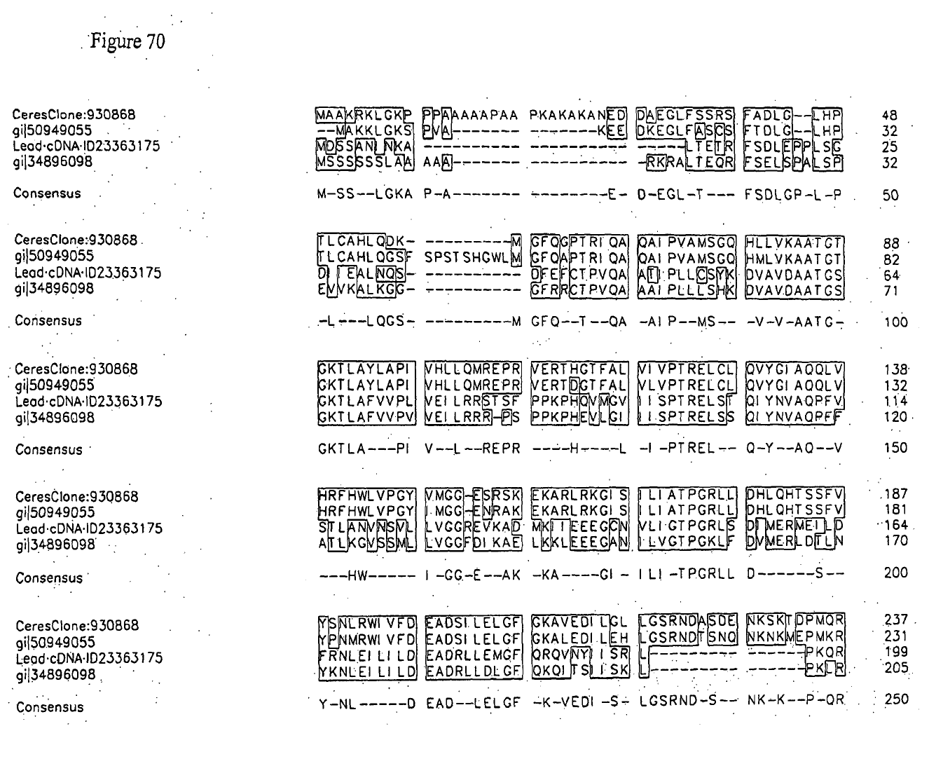

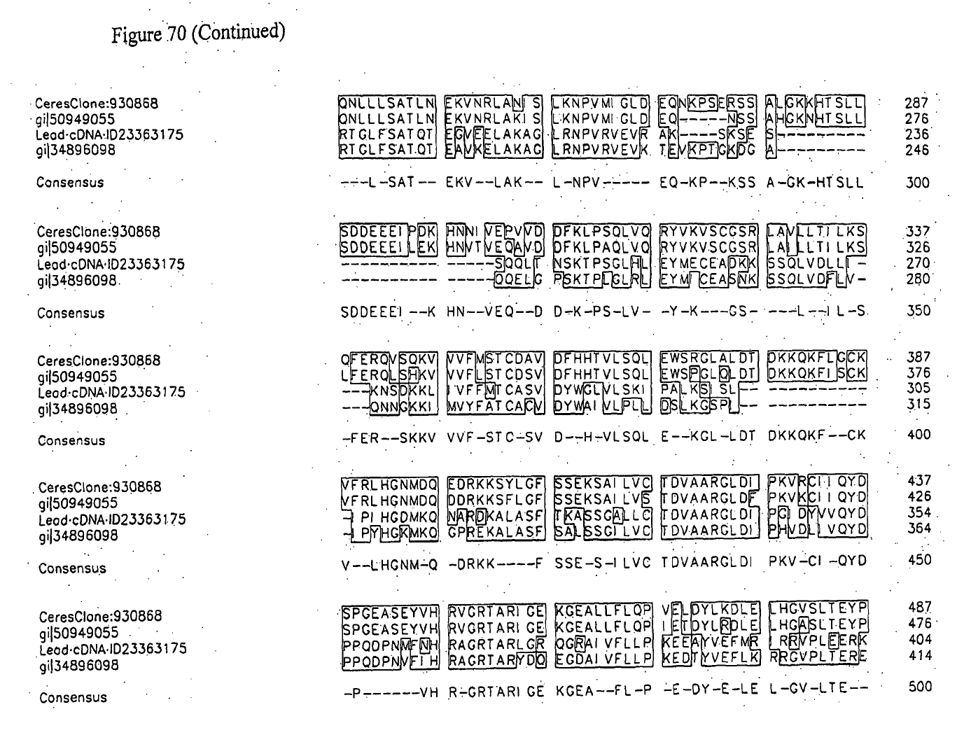

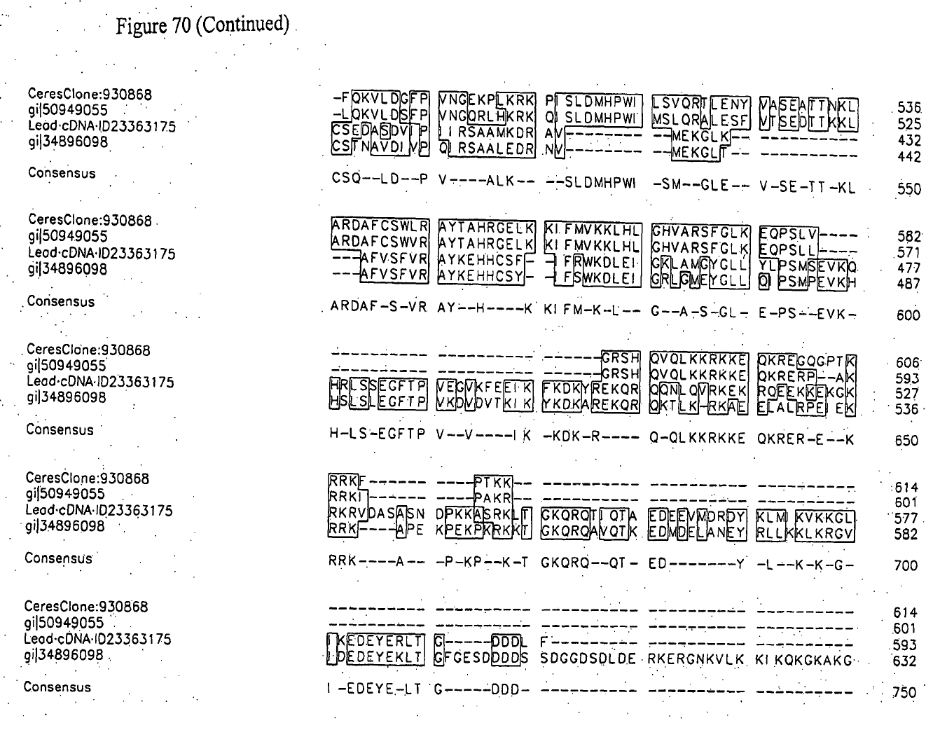

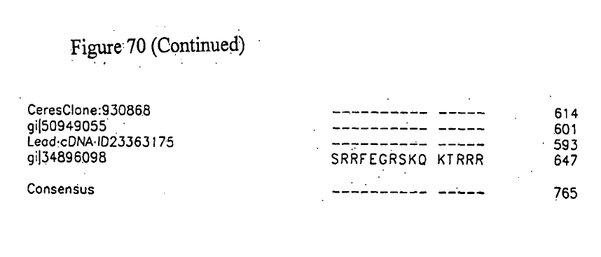

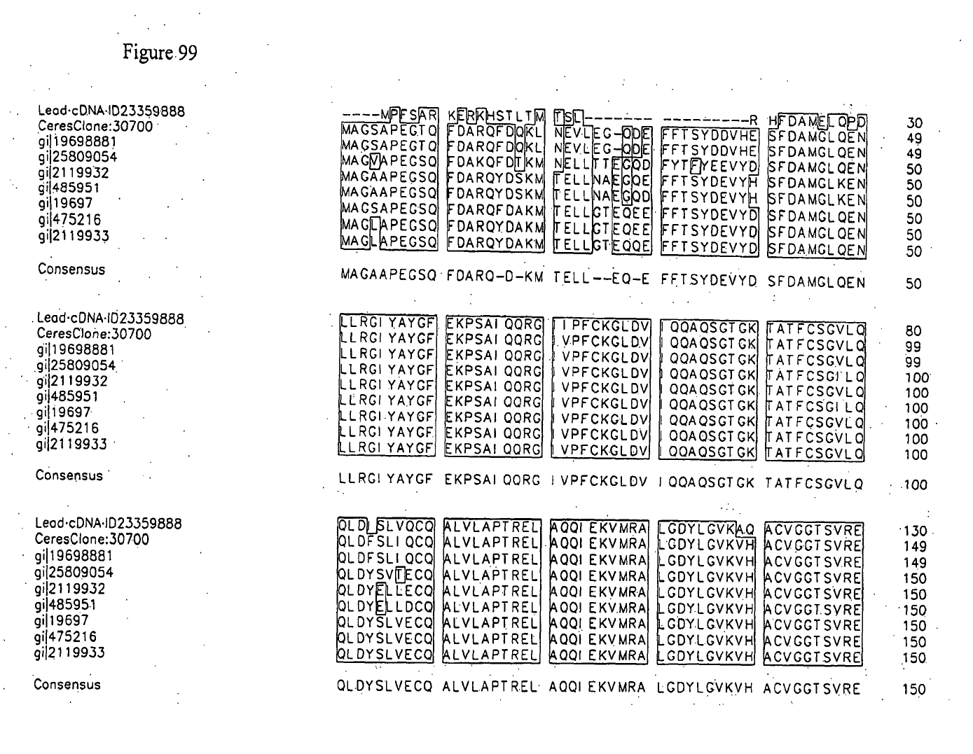

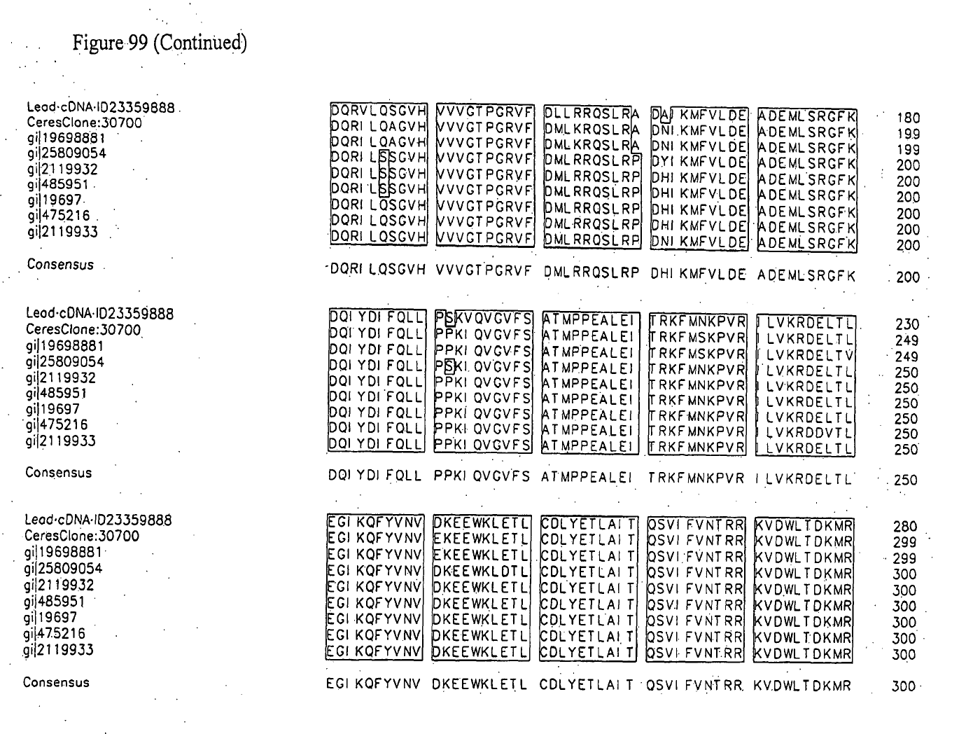

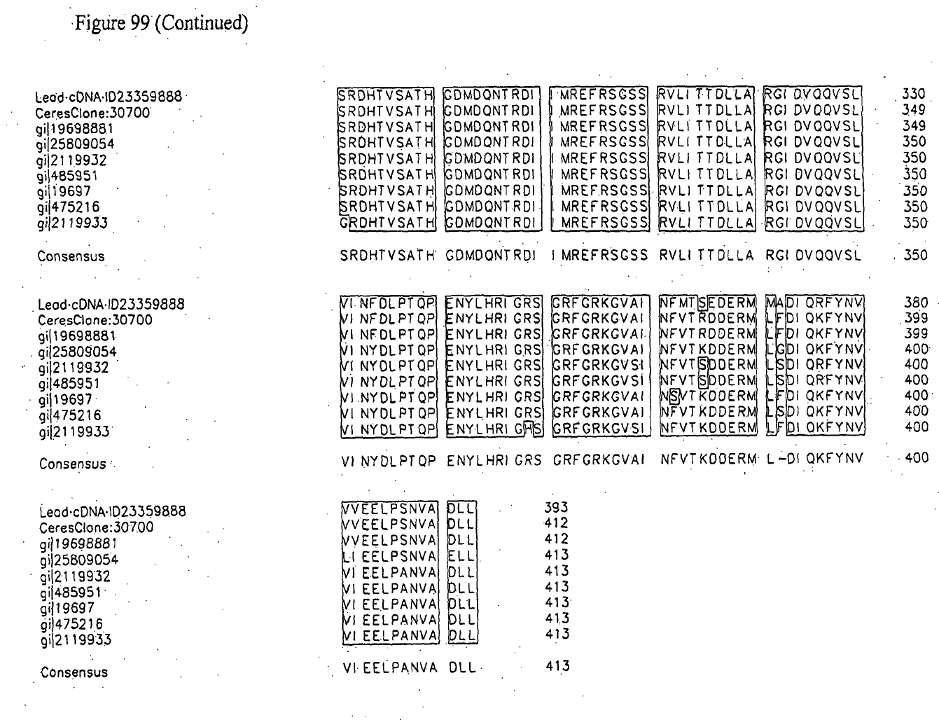

Amino acid sequences of homologs and/or orthologs of the polypeptide having the amino acid sequence set forth in SEQ ID NO:173, SEQ ID NO:711, and SEQ ID NO:1001 are provided in FIG. 10, FIG. 70, and FIG. 99, respectively.

For example, the alignment in FIG. 10 provides the amino acid sequences of cDNA ID 13653045 (5110A5; SEQ ID NO:173), gi|11385590_T (SEQ ID NO:180), gi|1385596_T (SEQ ID NO:181), gi|57899209_T (SEQ ID NO:182), CeresClone:1563222_T (SEQ ID NO:183), gi|11385602_T (SEQ ID NO:184), and gi|38564733_T (SEQ ID NO:185). Other homologs and/or orthologs of SEQ ID NO:173 include Public GI no. 11385590 (SEQ ID NO:174), Public GI no. 11385596 (SEQ ID NO:175), Public GI no. 57899209 (SEQ ID NO:176), Ceres CLONE ID no. 1563222 (SEQ ID NO:177), Public GI no. 11385602 (SEQ ID NO:178), and Public GI no. 38564733 (SEQ ID NO:179).

The alignment in FIG. 70 provides the amino acid sequences of cDNA ID 23363175 (SEQ ID NO:711), gi|34896098 (SEQ ID NO:712), CeresClone:930868 (SEQ ID NO:713), and gi|50949055 (SEQ ID NO:714).

The alignment in FIG. 99 provides the amino acid sequences of cDNA ID 23359888 (SEQ ID NO:1001), CeresClone:30700 (SEQ ID NO:1002), gi|19698881 (SEQ ID NO:1004), gi|19697 (SEQ ID NO:1005), gi|475216 (SEQ ID NO:1007), gi|2119932 (SEQ ID NO:1010), gi|2119933 (SEQ ID NO:1014), gi|485951 (SEQ ID NO:1015), and gi|25809054 (SEQ ID NO:1017). Other homologs and/or orthologs of SEQ ID NO:1001 include Public GI no. 23397033 (SEQ ID NO:1003), Public GI no. 21555870 (SEQ ID NO:1006), Public GI no. 2119938 (SEQ ID NO:1008), Public GI no. 2119934 (SEQ ID NO:1009), Public GI no. 485949 (SEQ ID NO:1011), Public GI no. 485945 (SEQ ID NO:1012), Public GI no. 485943 (SEQ ID NO:1013), and Public GI no. 485987 (SEQ ID NO:1016).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:174-185, SEQ ID NOs:712-714, SEQ ID NOs:1002-1017, or the consensus sequences set forth in FIG. 10, FIG. 70, or FIG. 99.

A regulatory protein can have a dsrm domain. The dsrm domain, or double-stranded RNA binding motif, is a putative motif shared by proteins that bind to dsRNA. Some DSRM proteins seem to bind to specific RNA targets. The dsrm motif is involved in localization of at least five different mRNAs in the early Drosophila embryo. SEQ ID NO:187 and SEQ ID NO:648 set forth the amino acid sequences of DNA clones, identified herein as cDNA ID 23477523 (SEQ ID NO:186) and cDNA ID 23517564 (SEQ ID NO:647), each of which is predicted to encode a polypeptide containing a dsrm domain.

A regulatory protein can comprise the amino acid sequence set forth in SEQ ID NO:187 or SEQ ID NO:648. Alternatively, a regulatory protein can be a homolog, ortholog, or variant of the polypeptide having the amino acid sequence set forth in SEQ ID NO:187 or SEQ ID NO:648. For example, a regulatory protein can have an amino acid sequence with at least 45% sequence identity, e.g., 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:187 or SEQ ID NO:648.

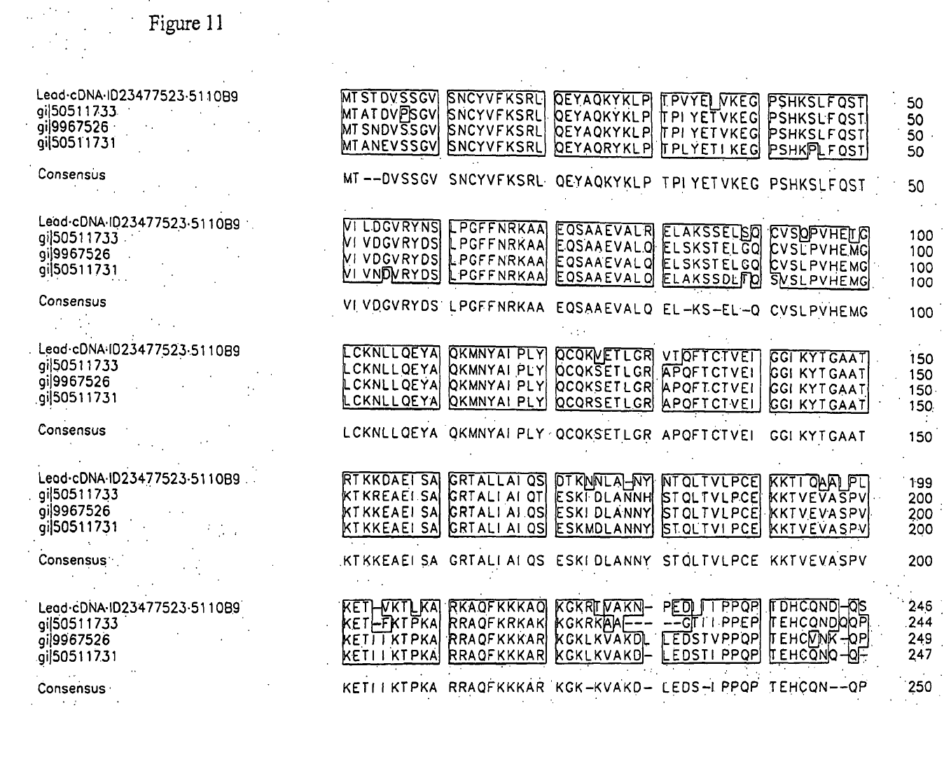

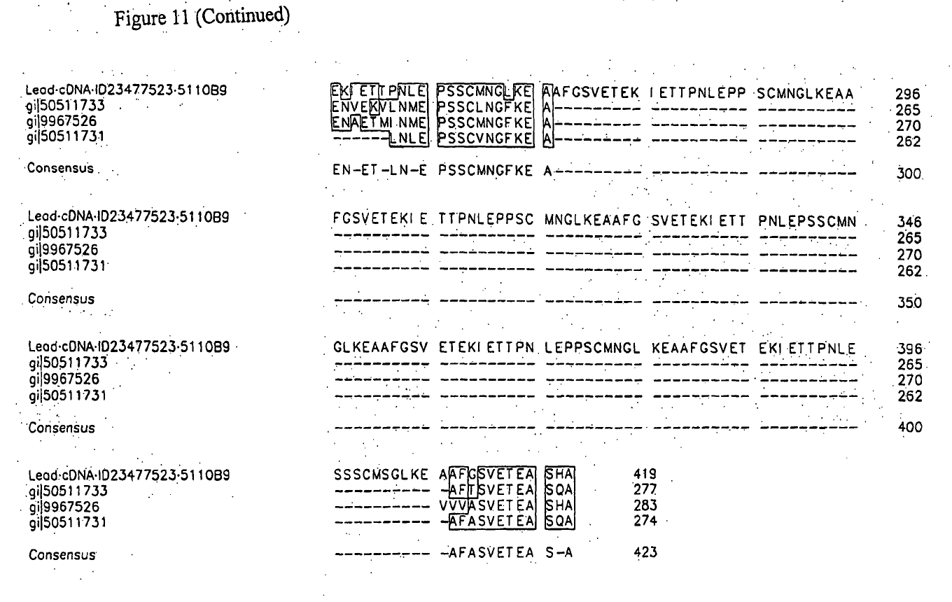

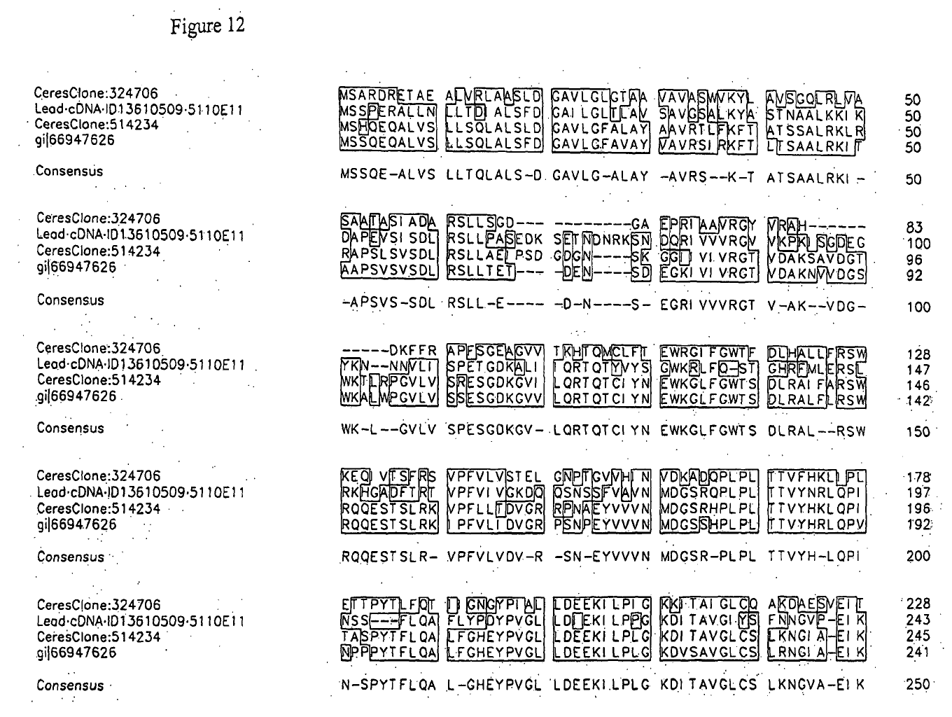

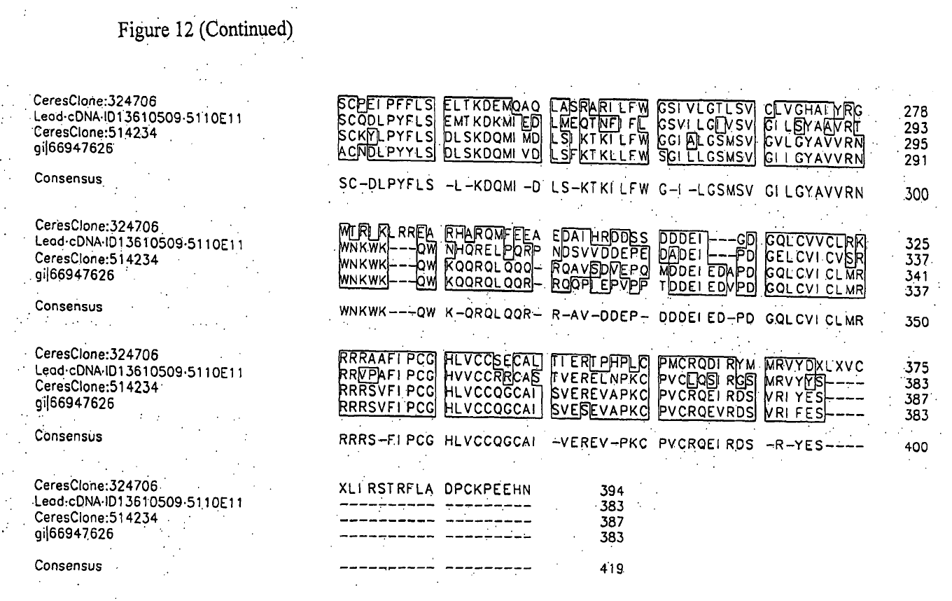

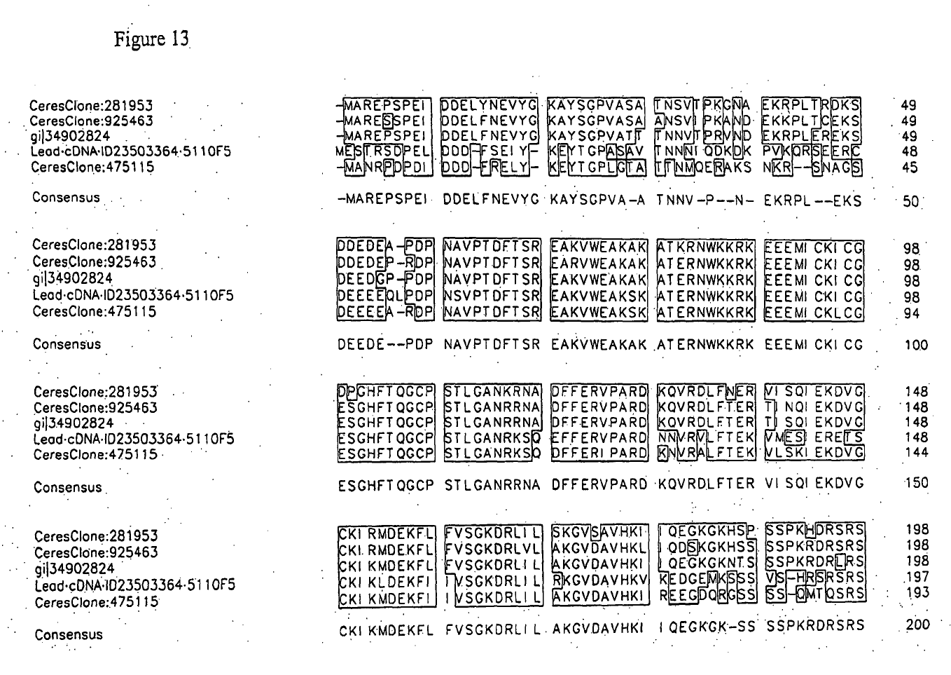

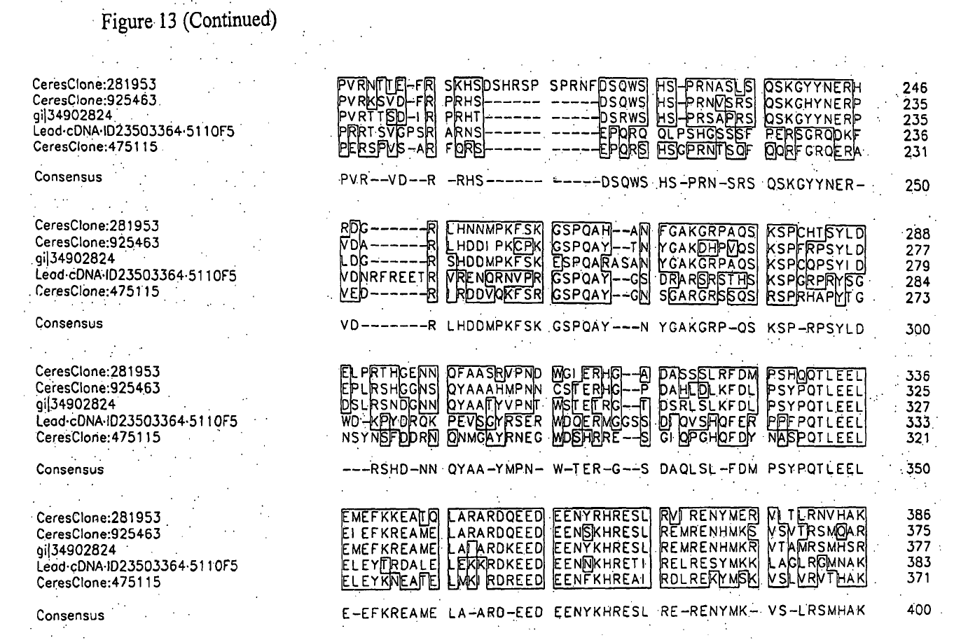

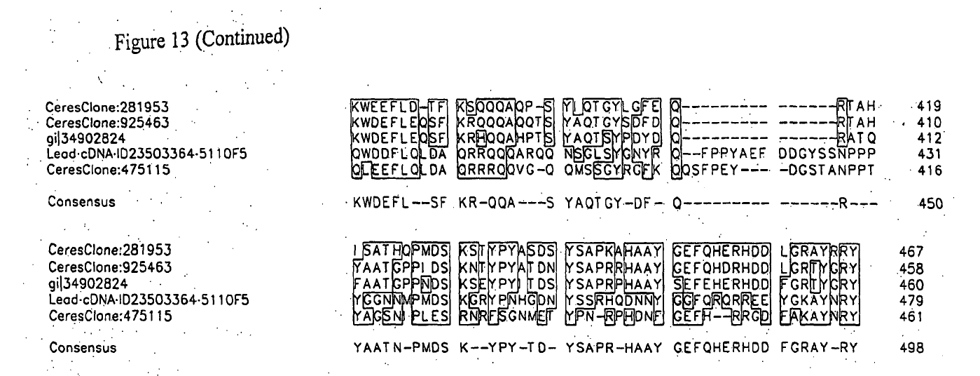

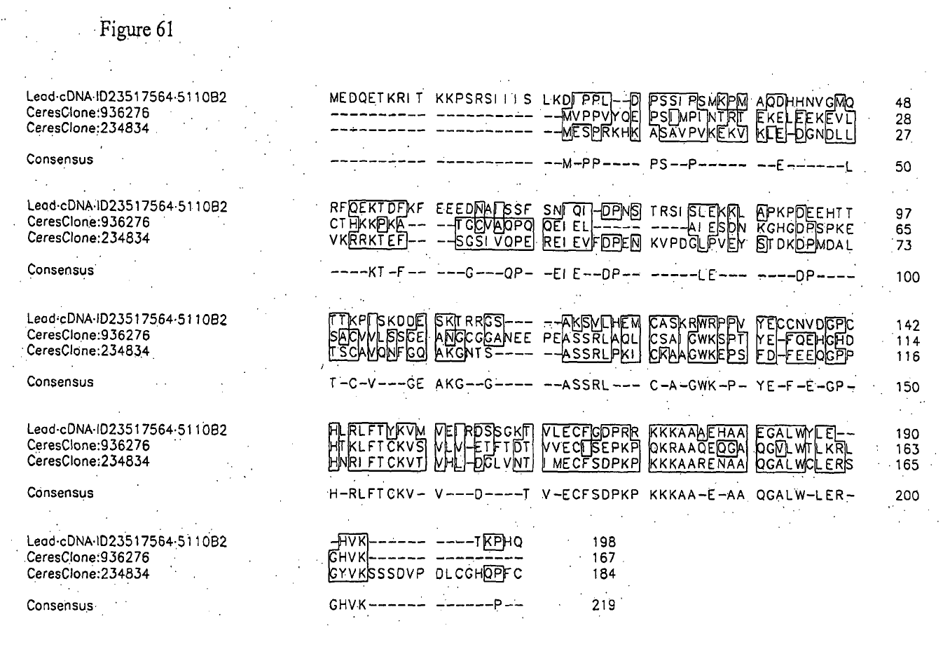

Amino acid sequences of homologs and/or orthologs of the polypeptide having the amino acid sequence set forth in SEQ ID NO:187 and SEQ ID NO:648 are provided in FIG. 11 and FIG. 61, respectively. Each of FIG. 11 and FIG. 61 also includes a consensus amino acid sequence determined by aligning homologous and/or orthologous amino acid sequences with the amino acid sequence set forth in SEQ ID NO:187 or SEQ ID NO:648, respectively.

For example, the alignment in FIG. 11 provides the amino acid sequences of cDNA ID 23477523 (5110B9; SEQ ID NO:187), gi|9967526 (SEQ ID NO:188), gi|50511733 (SEQ ID NO:189), and gi|5051731 (SEQ ID NO:190). Other homologs and/or orthologs of SEQ ID NO:187 include Public GI no. 50511725 (SEQ ID NO:191), Public GI no. 50511729 (SEQ ID NO:192), Public GI no. 50511727 (SEQ ID NO:193), Public GI no. 27262829 (SEQ ID NO:194), Public GI no. 27262839 (SEQ ID NO:195), Public GI no. 27262831 (SEQ ID NO:196), Public GI no. 27262837 (SEQ ID NO:197), and Public GI no. 27262833 (SEQ ID NO:198).

The alignment in FIG. 61 provides the amino acid sequences of cDNA ID 23517564 (5110B2; SEQ ID NO:648), CeresClone:936276 (SEQ ID NO:649), and CeresClone:234834 (SEQ ID NO:650).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:188-198, SEQ ID NOs:649-650, or the consensus sequences set forth in FIG. 11 or FIG. 61.

A regulatory protein can have a Mpp10 domain. The Mpp10 polypeptide family includes polypeptides related to Mpp10 (M phase phosphoprotein 10). The U3 small nucleolar ribonucleoprotein (snoRNP) is required for three cleavage events that generate the mature 18S rRNA from the pre-rRNA. SEQ ID NO:840 sets forth the amino acid sequence of a DNA clone, identified herein as cDNA ID 23505323 (SEQ ID NO:839), that is predicted to encode a polypeptide having a Mpp10 domain.

A regulatory protein can comprise the amino acid sequence set forth in SEQ ID NO:840. Alternatively, a regulatory protein can be a homolog, ortholog, or variant of the polypeptide having the amino acid sequence set forth in SEQ ID NO:840. For example, a regulatory protein can have an amino acid sequence with at least 45% sequence identity, e.g., 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:840.

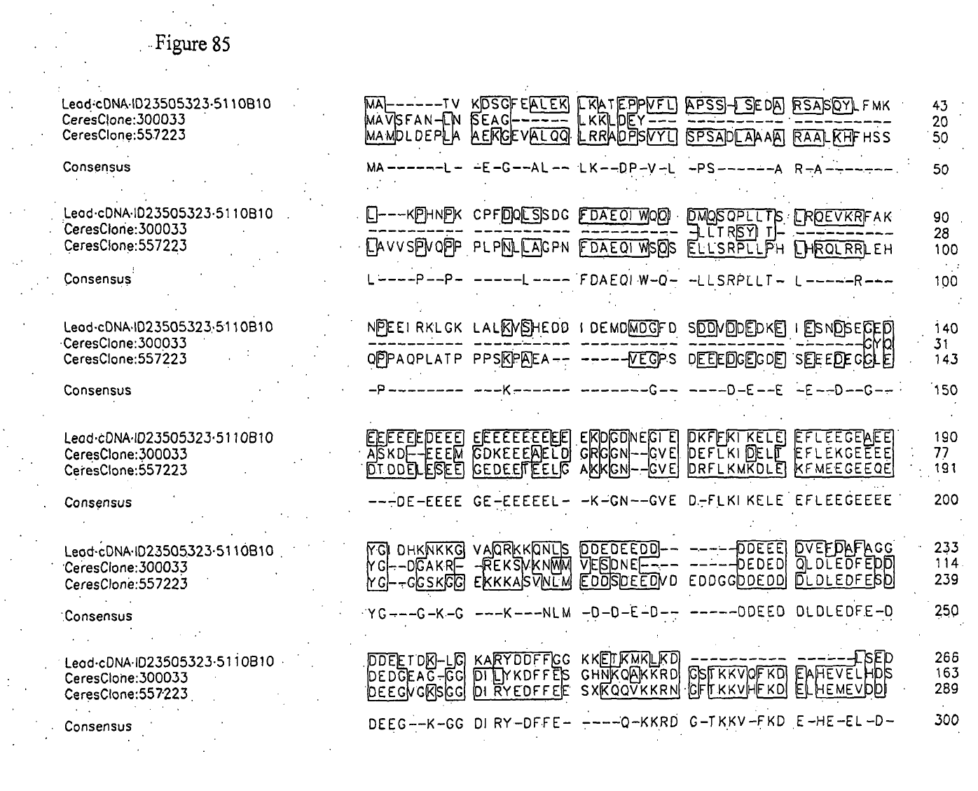

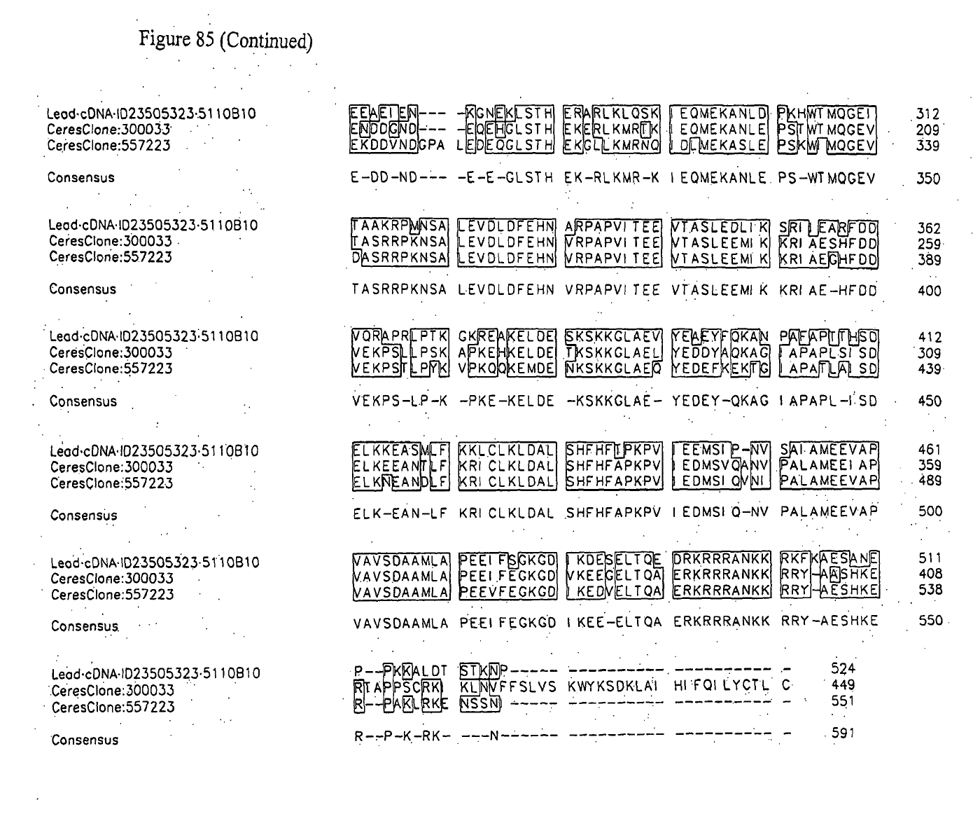

Amino acid sequences of homologs and/or orthologs of the polypeptide having the amino acid sequence set forth in SEQ ID NO:840 are provided in FIG. 85. FIG. 85 also includes a consensus amino acid sequence determined by aligning homologous and/or orthologous amino acid sequences with the amino acid sequence set forth in SEQ ID NO:840.

For example, the alignment in FIG. 85 provides the amino acid sequences of cDNA ID 23505323 (5110B10; SEQ ID NO:840), CeresClone:300033 (SEQ ID NO:842) and CeresClone:557223 (SEQ ID NO:843). Other homologs and/or orthologs of SEQ ID NO:840 include Ceres CLONE ID no. 15350 (SEQ ID NO:841).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:841-843 or the consensus sequence set forth in FIG. 85.

A regulatory protein can contain an AA_kinase domain and an ACT domain. The amino acid kinase (AA_kinase) family contains proteins with various specificities and includes the aspartate, glutamate, and uridylate kinase families. In prokaryotes and plants, the synthesis of the essential amino acids lysine and threonine is predominantly regulated by feed-back inhibition of aspartate kinase (AK) and dihydrodipicolinate synthase (DHPS). ACT domains generally have a regulatory role and are found in a wide range of metabolic enzymes that are regulated by amino acid concentration. Pairs of ACT domains bind specifically to a particular amino acid leading to regulation of the linked enzyme. The archetypical ACT domain is the C-terminal regulatory domain of 3-phosphoglycerate dehydrogenase (3PGDH), which folds with a ferredoxin-like topology. A pair of ACT domains forms an eight-stranded antiparallel sheet with two molecules of the allosteric inhibitor serine bound in the interface. SEQ ID NO:1321 sets forth the amino acid sequence of a DNA clone, identified herein as cDNA ID 23389279 (SEQ ID NO:1320), that is predicted to encode a polypeptide containing an AA_kinase domain and an ACT domain.

A regulatory protein can comprise the amino acid sequence set forth in SEQ ID NO:1321. Alternatively, a regulatory protein can be a homolog, ortholog, or variant of the polypeptide having the amino acid sequence set forth in SEQ ID NO:1321. For example, a regulatory protein can have an amino acid sequence with at least 40% sequence identity, e.g., 40%, 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:1321.

A regulatory protein can contain an NHL repeat. The NHL (NCL-1, HT2A and LIN-41) repeat is found in a variety of enzymes of the copper type II, ascorbate-dependent monooxygenase family, which catalyze the C-terminal alpha-amidation of biological peptides. The repeat also occurs in a human zinc finger protein that specifically interacts with the activation domain of lentiviral Tat proteins. The repeat domain is often associated with RING finger and B-box motifs. SEQ ID NO:812 sets forth the amino acid sequence of a DNA clone, identified herein as cDNA ID 23768927 (SEQ ID NO:811), that is predicted to encode a polypeptide containing an NHL domain.

A regulatory protein can comprise the amino acid sequence set forth in SEQ ID NO:812. Alternatively, a regulatory protein can be a homolog, ortholog, or variant of the polypeptide having the amino acid sequence set forth in SEQ ID NO:812. For example, a regulatory protein can have an amino acid sequence with at least 35% sequence identity, e.g., 35%, 40%, 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:812.

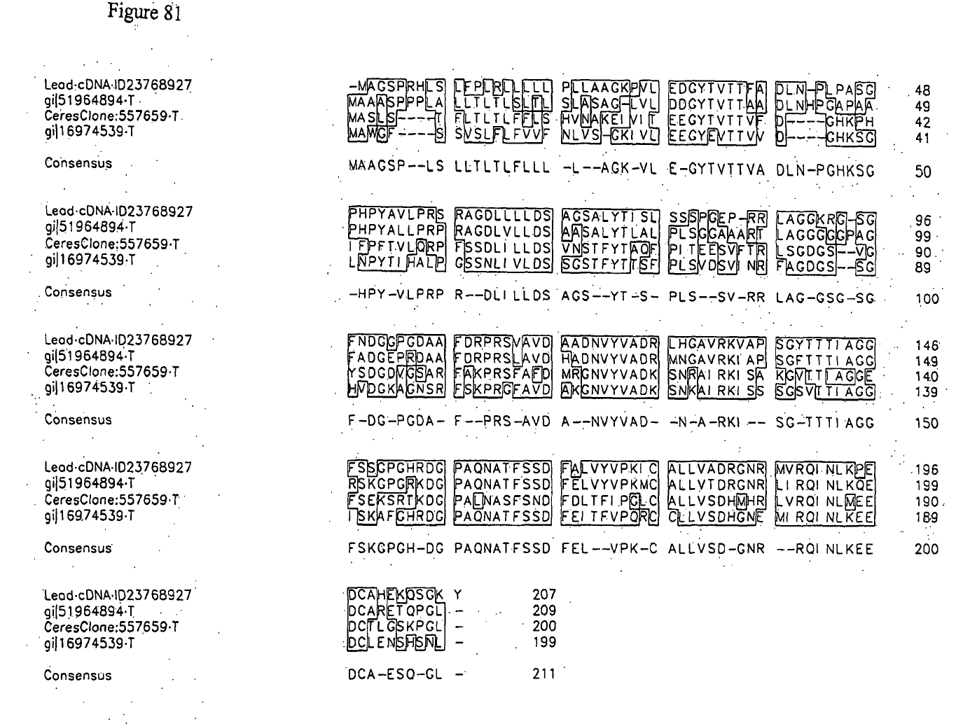

Amino acid sequences of homologs and/or orthologs of the polypeptide having the amino acid sequence set forth in SEQ ID NO:812 are provided in FIG. 81. FIG. 81 also includes a consensus amino acid sequence determined by aligning homologous and/or orthologous amino acid sequences with the amino acid sequence set forth in SEQ ID NO:812.

For example, the alignment in FIG. 81 provides the amino acid sequences of cDNA ID 23768927 (SEQ ID NO:812), gi|51964894_T (SEQ ID NO:816), gi|16974539_T (SEQ ID NO:817), and CeresClone:557659_T (SEQ ID NO:818). Other homologs and/or orthologs of SEQ ID NO:812 include Public GI no. 51964894 (SEQ ID NO:813), Public GI no. 16974539 (SEQ ID NO:814), and Ceres CLONE ID no. 557659 (SEQ ID NO:815).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:813-818 or the consensus sequence set forth in FIG. 81.

A regulatory protein can contain a Usp domain characteristic of a polypeptide belonging to the universal stress protein family. The universal stress protein UspA is a small cytoplasmic bacterial protein whose expression is enhanced when the cell is exposed to stress agents. UspA enhances the rate of cell survival during prolonged exposure to such conditions, and may provide a general “stress endurance” activity. SEQ ID NO:1192 sets forth the amino acid sequence of a DNA clone, identified herein as cDNA ID 23416869 (SEQ ID NO:1191), that is predicted to encode a polypeptide containing a Usp domain.

A regulatory protein can comprise the amino acid sequence set forth in SEQ ID NO:1192. Alternatively, a regulatory protein can be a homolog, ortholog, or variant of the polypeptide having the amino acid sequence set forth in SEQ ID NO:1192. For example, a regulatory protein can have an amino acid sequence with at least 45% sequence identity, e.g., 45%, 47%, 48%, 49%, 50%, 51%, 52%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:1192.

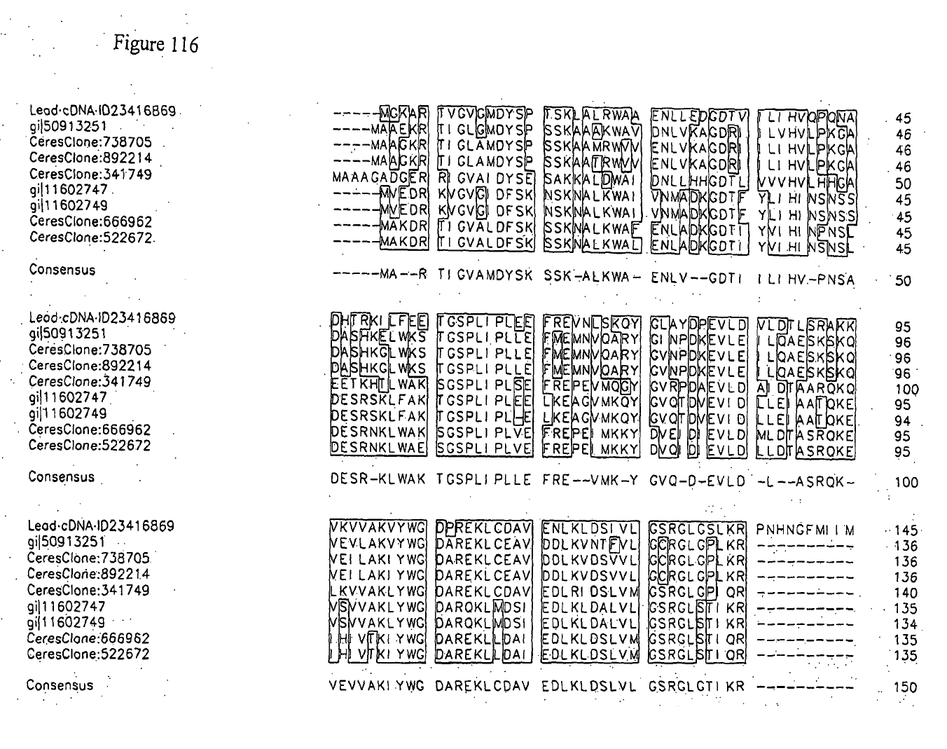

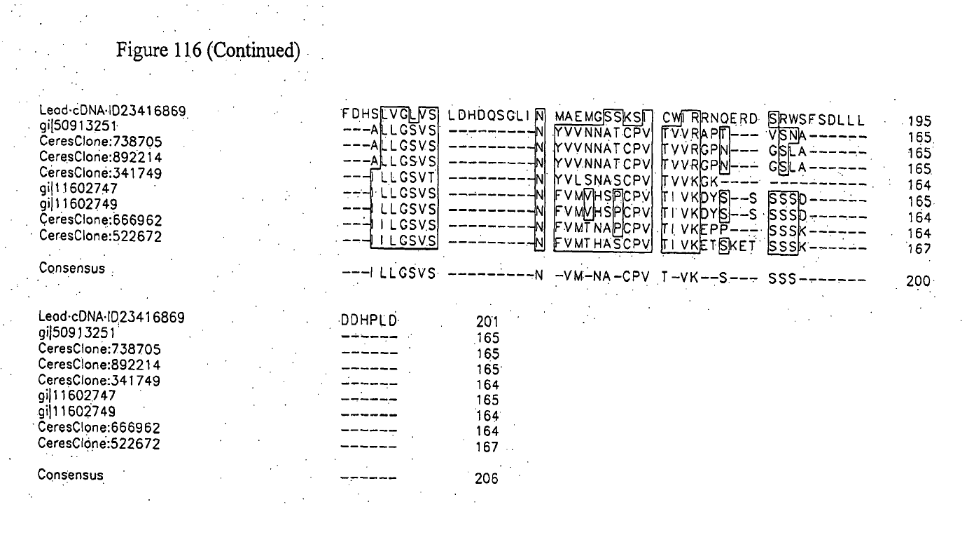

Amino acid sequences of homologs and/or orthologs of the polypeptide having the amino acid sequence set forth in SEQ ID NO:1192 are provided in FIG. 116. FIG. 116 also includes a consensus amino acid sequence determined by aligning homologous and/or orthologous amino acid sequences with the amino acid sequence set forth in SEQ ID NO:1192.

For example, the alignment in FIG. 116 provides the amino acid sequences of cDNA ID 23416869 (SEQ ID NO:1192), CeresClone:738705 (SEQ ID NO:1193), CeresClone:892214 (SEQ ID NO:1194), gi|50913251 (SEQ ID NO:1195), CeresClone:341749 (SEQ ID NO:1196), CeresClone:666962 (SEQ ID NO:1197), CeresClone:522672 (SEQ ID NO:1198), gi|11602747 (SEQ ID NO:1199), and gi|11602749 (SEQ ID NO:1200).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:1193-1200 or the consensus sequence set forth in FIG. 116.

A regulatory protein can contain an Rm1D substrate binding domain. L-rhamnose is a saccharide required for the virulence of some bacteria. Its precursor, dTDP-L-rhamnose, is synthesized by four different enzymes, the final one of which is Rm1D. The Rm1D substrate binding domain is responsible for binding a sugar nucleotide. SEQ ID NO:1429 sets forth the amino acid sequence of a DNA clone, identified herein as cDNA ID 23699979 (SEQ ID NO:1428), that is predicted to encode a polypeptide containing an Rm1D substrate binding domain.

A regulatory protein can comprise the amino acid sequence set forth in SEQ ID NO:1429. Alternatively, a regulatory protein can be a homolog, ortholog, or variant of the polypeptide having the amino acid sequence set forth in SEQ ID NO:1429. For example, a regulatory protein can have an amino acid sequence with at least 55% sequence identity, e.g., 55%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:1429.

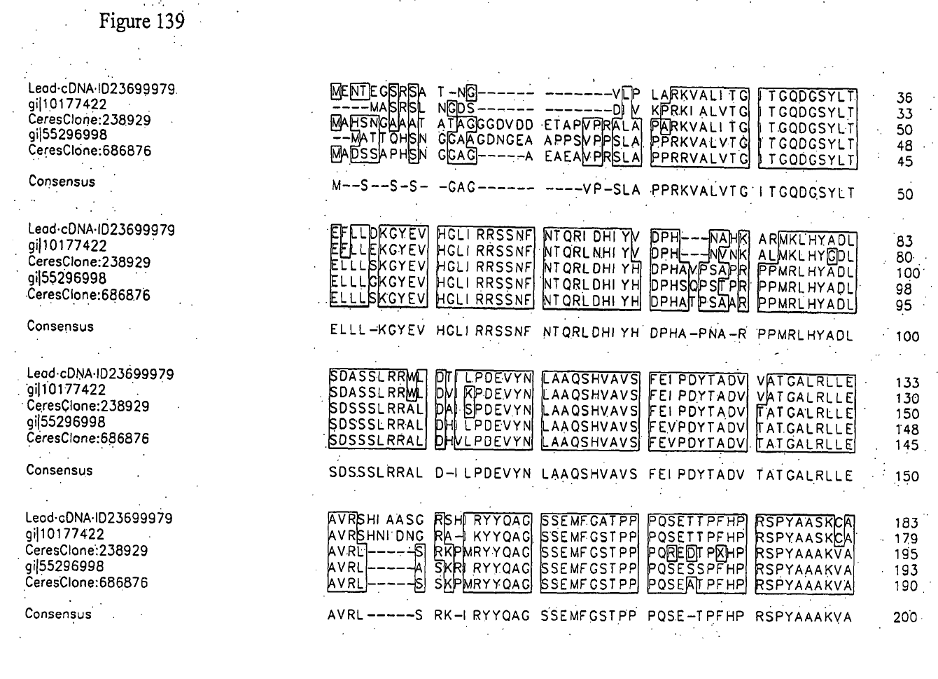

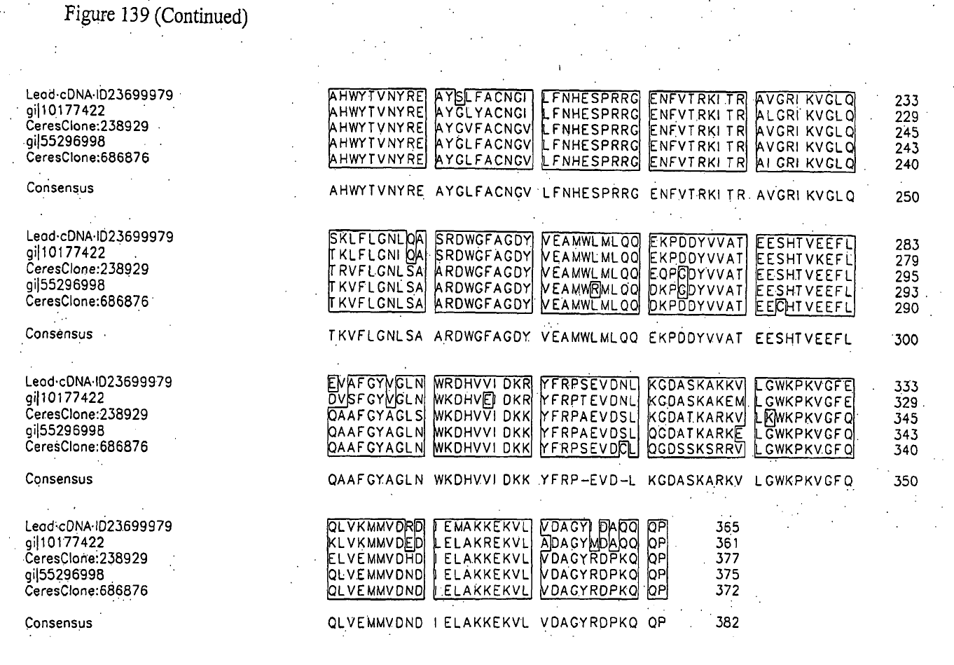

Amino acid sequences of homologs and/or orthologs of the polypeptide having the amino acid sequence set forth in SEQ ID NO:1429 are provided in FIG. 139. FIG. 139 also includes a consensus amino acid sequence determined by aligning homologous and/or orthologous amino acid sequences with the amino acid sequence set forth in SEQ ID NO:1429.

For example, the alignment in FIG. 139 provides the amino acid sequences of cDNA ID 23699979 (SEQ ID NO:1429), gi|10177422 (SEQ ID NO:1430), gi|55296998 (SEQ ID NO:1436), CeresClone:238929 (SEQ ID NO:1437), and CeresClone:686876 (SEQ ID NO:1438). Other homologs and/or orthologs of SEQ ID NO:1429 include Public GI no. 1764100 (SEQ ID NO:1431), Public GI no. 28373943 (SEQ ID NO:1432), Ceres CLONE ID no. 11217 (SEQ ID NO:1433), Public GI no. 21536808 (SEQ ID NO:1434), and Public GI no. 6562268 (SEQ ID NO:1435).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:1430-1438 or the consensus sequence set forth in FIG. 139.

A regulatory protein can contain an X8 domain. The X8 domain contains six conserved cysteine residues that presumably form three disulphide bridges. The X8 domain is found in an Olive pollen allergen as well as at the C-terminus of family 17 glycosyl hydrolases. This domain may be involved in carbohydrate binding. SEQ ID NO:732 sets forth the amino acid sequence of a DNA clone, identified herein as cDNA ID 23751471 (SEQ ID NO:731), that is predicted to encode a polypeptide containing an X8 domain.

A regulatory protein can comprise the amino acid sequence set forth in SEQ ID NO:732. Alternatively, a regulatory protein can be a homolog, ortholog, or variant of the polypeptide having the amino acid sequence set forth in SEQ ID NO:732. For example, a regulatory protein can have an amino acid sequence with at least 35% sequence identity, e.g., 35%, 40%, 45%, 50%, 55%, 56%, 57%, 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:732.

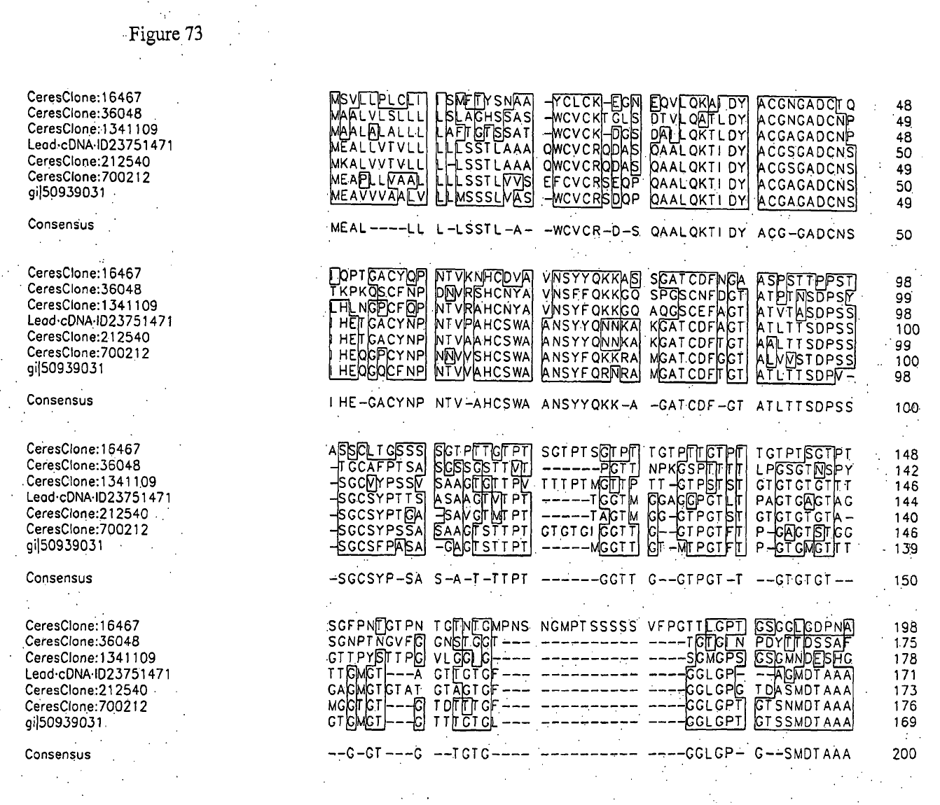

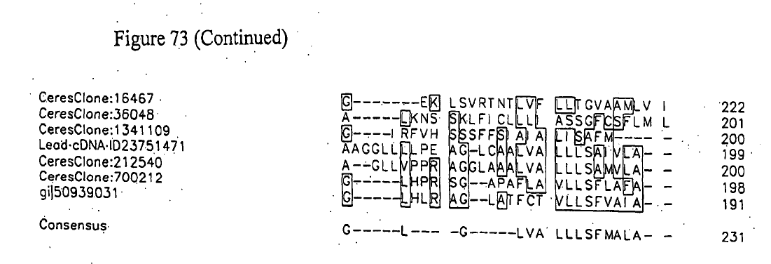

Amino acid sequences of homologs and/or orthologs of the polypeptide having the amino acid sequence set forth in SEQ ID NO:732 are provided in FIG. 73. FIG. 73 also includes a consensus amino acid sequence determined by aligning homologous and/or orthologous amino acid sequences with the amino acid sequence set forth in SEQ ID NO:732.

For example, the alignment in FIG. 73 provides the amino acid sequences of cDNA ID 23751471 (SEQ ID NO:732), CeresClone:212540 (SEQ ID NO:733), gi|50939031 (SEQ ID NO:734), CeresClone:700212 (SEQ ID NO:735), CeresClone:1341109 (SEQ ID NO:736), CeresClone:16467 (SEQ ID NO:740), and CeresClone:36048 (SEQ ID NO:746). Other homologs and/or orthologs of SEQ ID NO:732 include Ceres CLONE ID no. 517837 (SEQ ID NO:737), Public GI no. 16323412 (SEQ ID NO:738), Public GI no. 21553768 (SEQ ID NO:739), Public GI no. 51970462 (SEQ ID NO:741), Public GI no. 21592859 (SEQ ID NO:742), Ceres CLONE ID no. 33347 (SEQ ID NO:743), Public GI no. 26452180 (SEQ ID NO:744), and Public GI no. 9759459 (SEQ ID NO:745).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:733-746 or the consensus sequence set forth in FIG. 73.

A regulatory protein can contain a PsbP domain. The PsbP polypeptide family consists of the 23 kDa subunit of oxygen evolving system of photosystem II or PsbP from various plants (where it is encoded by the nuclear genome) and Cyanobacteria. Both PsbP and PsbQ are regulators that are necessary for the biogenesis of optically active PSII. The 23 kDa PsbP protein is required for PSII to be fully operational in vivo. PsbP increases the affinity of the water oxidation site for chloride ions and provides the conditions required for high affinity binding of calcium ions. PsbP is encoded in the nuclear genome in plants. SEQ ID NO:1382 sets forth the amino acid sequence of a DNA clone, identified herein as cDNA ID 23367406 (SEQ ID NO:1381), that is predicted to encode a polypeptide containing a PsbP domain.

A regulatory protein can comprise the amino acid sequence set forth in SEQ ID NO:1382. Alternatively, a regulatory protein can be a homolog, ortholog, or variant of the polypeptide having the amino acid sequence set forth in SEQ ID NO:1382. For example, a regulatory protein can have an amino acid sequence with at least 75% sequence identity, e.g., 75%, 80%, 85%, 90%, 95%, 97%, 98%, or 99% sequence identity, to the amino acid sequence set forth in SEQ ID NO:1382.

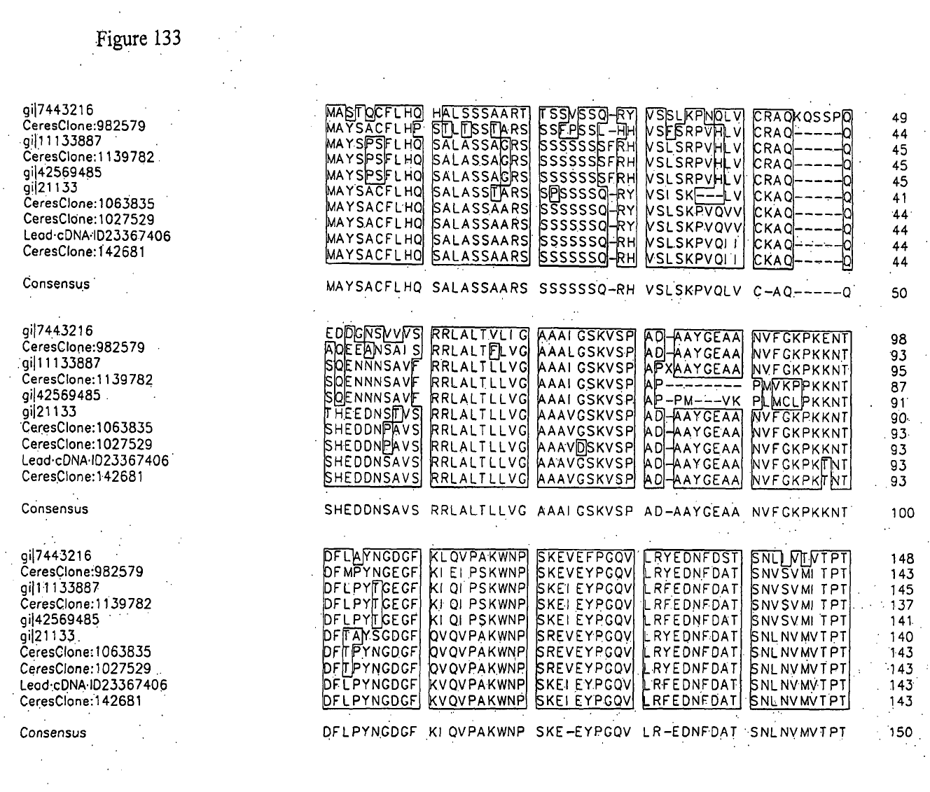

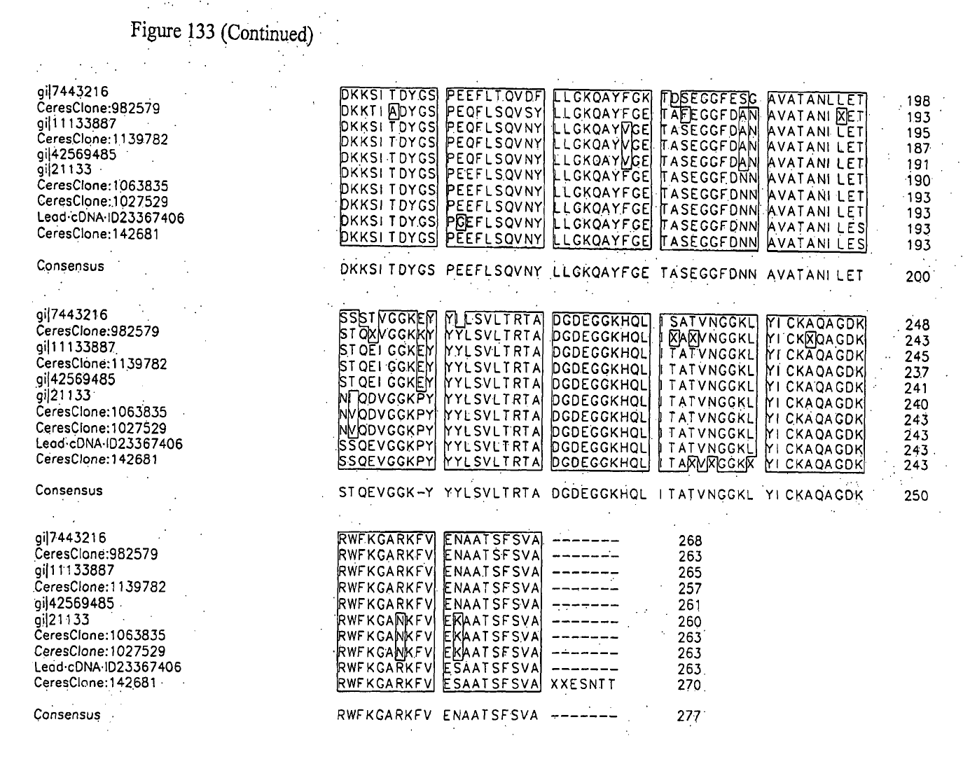

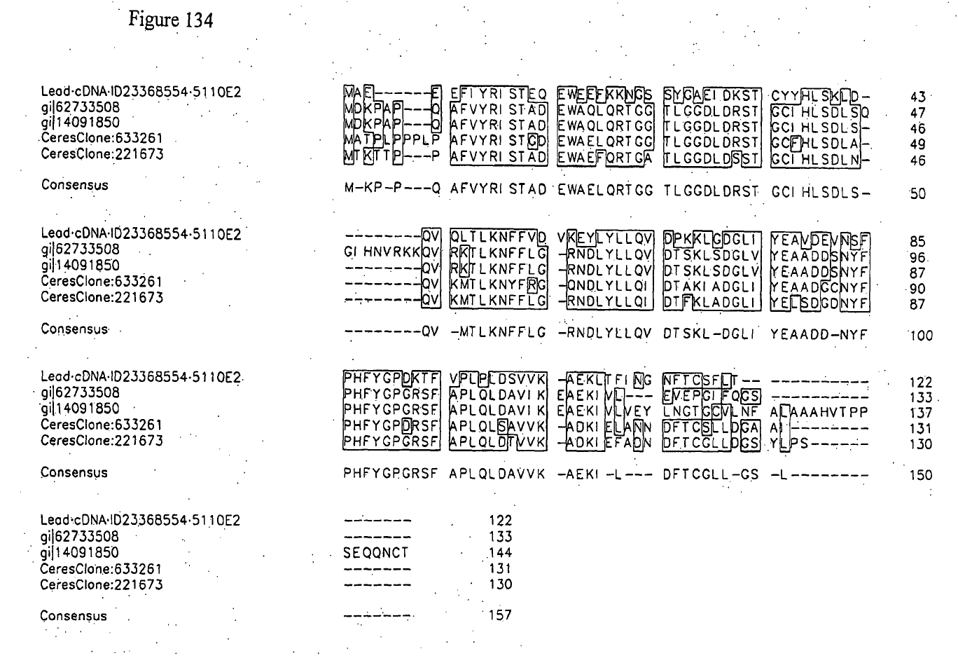

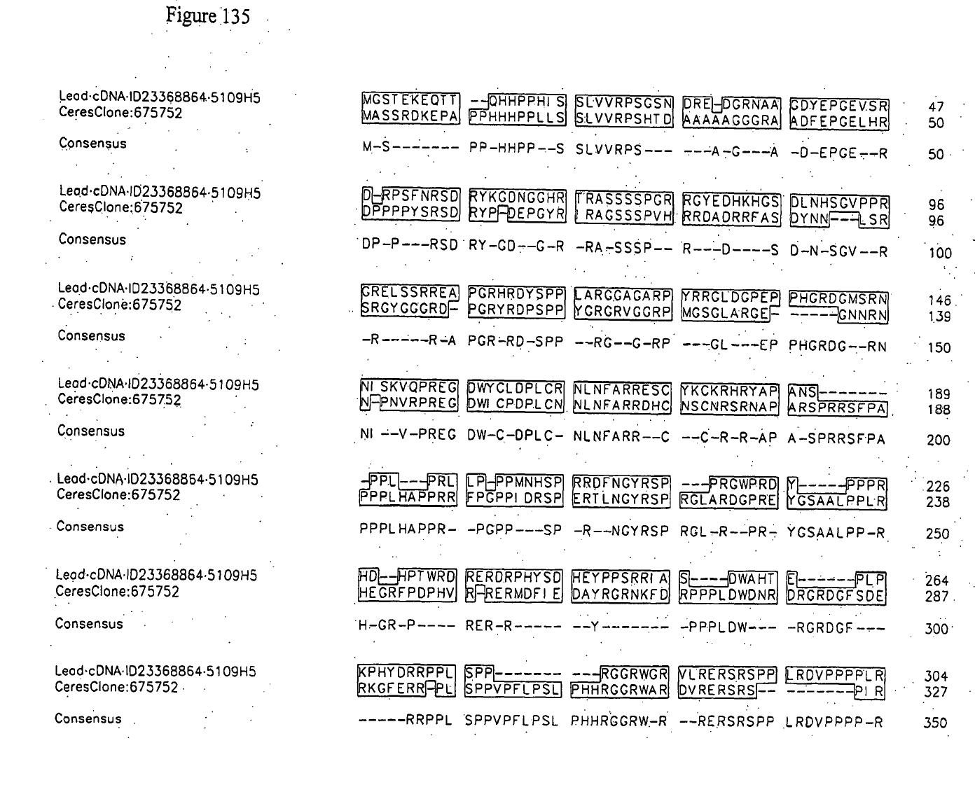

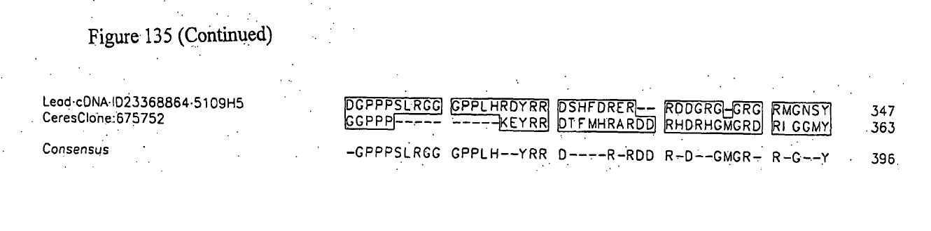

Amino acid sequences of homologs and/or orthologs of the polypeptide having the amino acid sequence set forth in SEQ ID NO:1382 are provided in FIG. 133. FIG. 133 also includes a consensus amino acid sequence determined by aligning homologous and/or orthologous amino acid sequences with the amino acid sequence set forth in SEQ ID NO:1382.

For example, the alignment in FIG. 133 provides the amino acid sequences of cDNA ID 23367406 (SEQ ID NO:1382), CeresClone:142681 (SEQ ID NO:1383), CeresClone:1063835 (SEQ ID NO:1384), CeresClone:1027529 (SEQ ID NO:1385), gi|21133 (SEQ ID NO:1386), gi|11133887 (SEQ ID NO:1387), CeresClone:1139782 (SEQ ID NO:1388), gi|42569485 (SEQ ID NO:1390), CeresClone:982579 (SEQ ID NO:1391), and gi|7443216 (SEQ ID NO:1392). Other homologs and/or orthologs of SEQ ID NO:1382 include Public GI no. 2880056 (SEQ ID NO:1389).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:1383-1392 or the consensus sequence set forth in FIG. 133.

A regulatory protein can contain a p450 domain characteristic of a cytochrome P450 polypeptide. The cytochrome P450 enzymes constitute a superfamily of haemthiolate proteins. P450 enzymes usually act as terminal oxidases in multicomponent electron transfer chains, called P450-containing monooxygenase systems, and are involved in metabolism of a plethora of both exogenous and endogenous compounds. The conserved core is composed of a coil referred to as the “meander,” a four-helix bundle, helices J and K, and two sets of beta-sheets. These regions constitute the haem-binding loop (with an absolutely conserved cysteine that serves as the 5th ligand for the haem iron), the proton-transfer groove, and the absolutely conserved EXXR motif in helix K. SEQ ID NO:1423 sets forth the amino acid sequence of a DNA clone, identified herein as cDNA ID 23516818 (SEQ ID NO:1422), that is predicted to encode a polypeptide containing a p450 domain.

A regulatory protein can comprise the amino acid sequence set forth in SEQ ID NO:1423. Alternatively, a regulatory protein can be a homolog, ortholog, or variant of the polypeptide having the amino acid sequence set forth in SEQ ID NO:1423. For example, a regulatory protein can have an amino acid sequence with at least 65% sequence identity, e.g., 65%, 70%, 75%, 80%, 85%, 90%, 95%, 97%, 98%, or 99% sequence identity, to the amino acid sequence set forth in SEQ ID NO:1423.

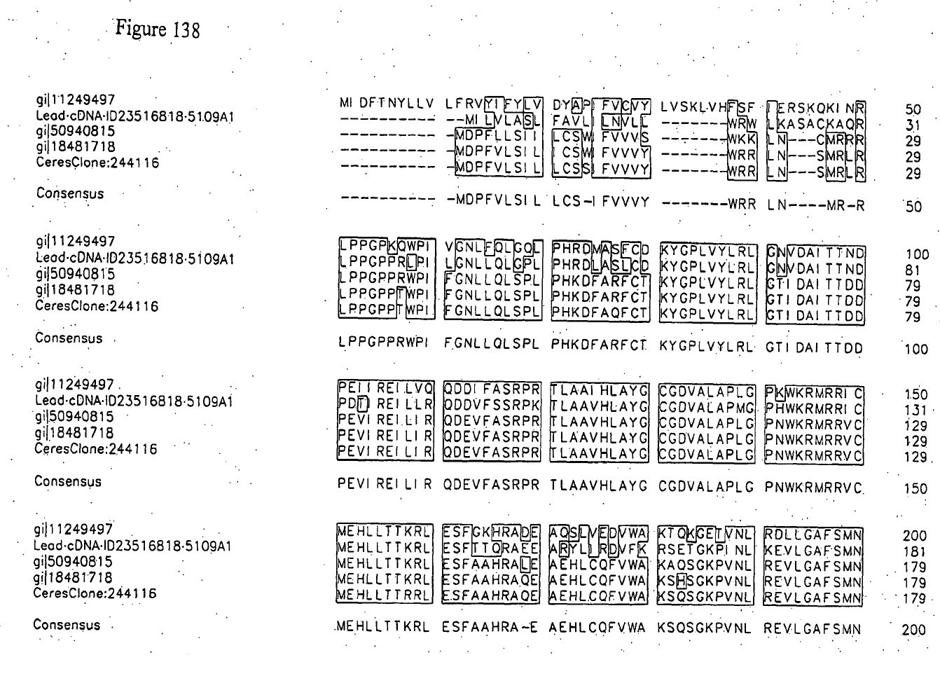

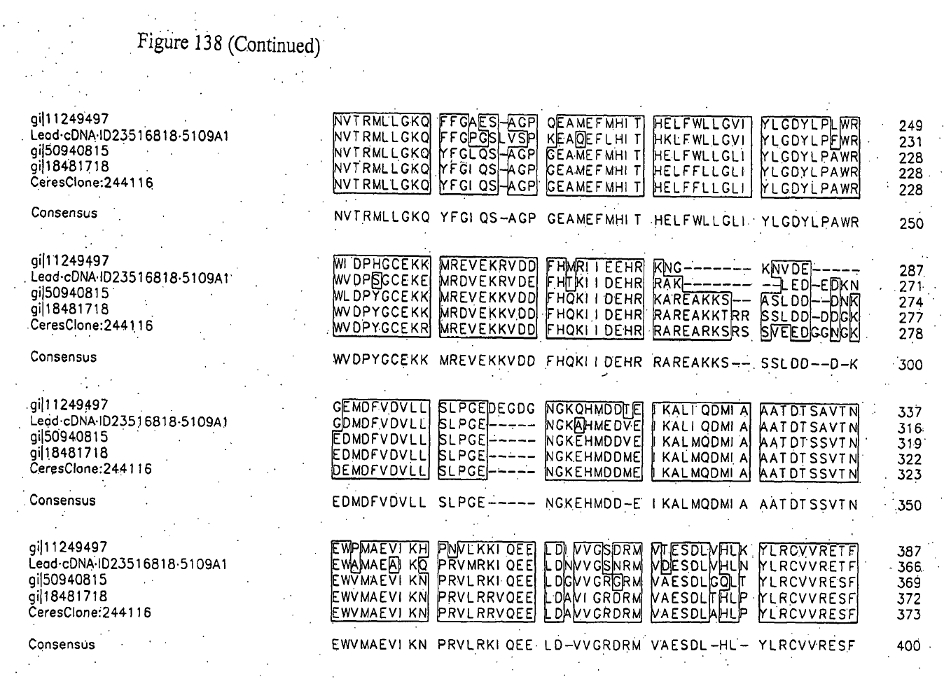

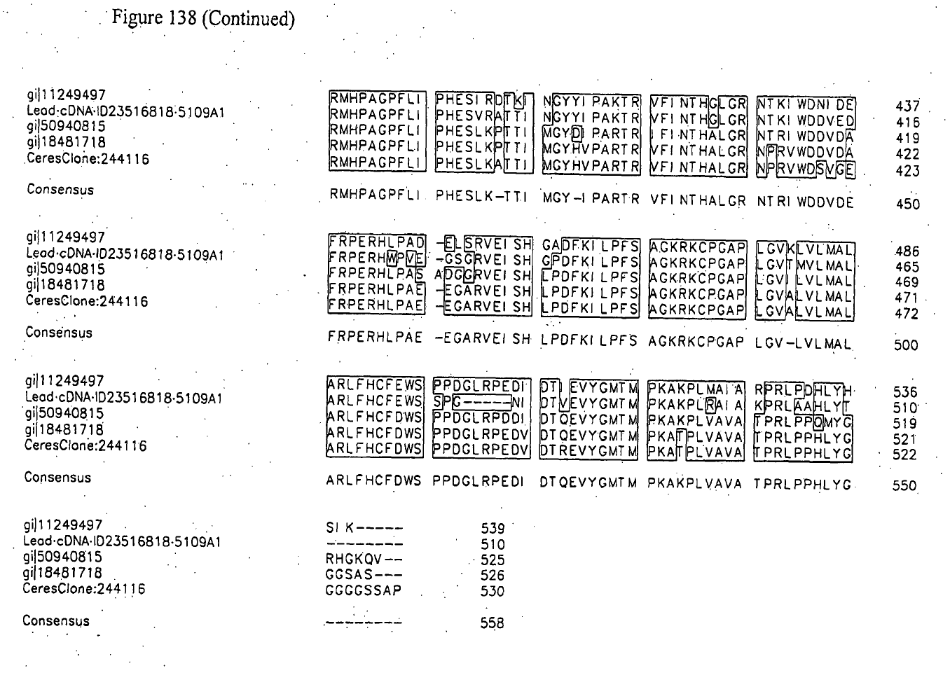

Amino acid sequences of homologs and/or orthologs of the polypeptide having the amino acid sequence set forth in SEQ ID NO:1423 are provided in FIG. 138. FIG. 138 also includes a consensus amino acid sequence determined by aligning homologous and/or orthologous amino acid sequences with the amino acid sequence set forth in SEQ ID NO:1423.

For example, the alignment in FIG. 138 provides the amino acid sequences of cDNA ID 23516818 (5109A1; SEQ ID NO:1423), gi|11249497 (SEQ ID NO:1424), gi|50940815 (SEQ ID NO:1425), gi|18481718 (SEQ ID NO:1426), and CeresClone:244116 (SEQ ID NO:1427).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:1424-1427 or the consensus sequence set forth in FIG. 138.

A regulatory protein can contain a zf-Tim10_DDP domain characteristic of a Tim10/DDP family zinc finger polypeptide. Members of the Tim10/DDP family contain a putative zinc binding domain with four conserved cysteine residues. The zf-Tim10_DDP domain is found in the human disease protein Deafness Dystonia Protein 1. Members of the Tim10/DDP family, such as Tim9 and Tim10, are involved in mitochondrial protein import. SEQ ID NO:1042 sets forth the amino acid sequence of a DNA clone, identified herein as cDNA ID 23386664 (SEQ ID NO:1041), that is predicted to encode a Tim 10/DDP family zinc finger polypeptide.

A regulatory protein can comprise the amino acid sequence set forth in SEQ ID NO:1042. Alternatively, a regulatory protein can be a homolog, ortholog, or variant of the polypeptide having the amino acid sequence set forth in SEQ ID NO:1042. For example, a regulatory protein can have an amino acid sequence with at least 30% sequence identity, e.g., 30%, 35%, 40%, 45%, 50%, 55%, 60%, 65%, 70%, 75%, 80%, 85%, 90%, 95%, 97%, 98%, or 99% sequence identity, to the amino acid sequence set forth in SEQ ID NO:1042.

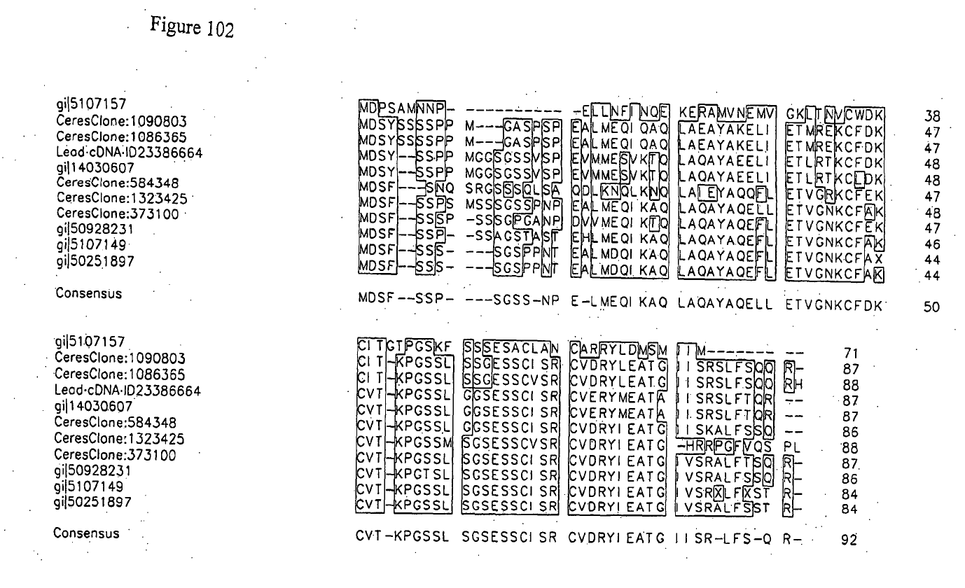

Amino acid sequences of homologs and/or orthologs of the polypeptide having the amino acid sequence set forth in SEQ ID NO:1042 are provided in FIG. 102. FIG. 102 also includes a consensus amino acid sequence determined by aligning homologous and/or orthologous amino acid sequences with the amino acid sequence set forth in SEQ ID NO:1042.

For example, the alignment in FIG. 102 provides the amino acid sequences of cDNA ID 23386664 (SEQ ID NO:1042), gi|14030607 (SEQ ID NO:1043), CeresClone:1090803 (SEQ ID NO:1045), CeresClone:1086365 (SEQ ID NO:1047), CeresClone:1323425 (SEQ ID NO:1048), CeresClone:373100 (SEQ ID NO:1050), gi|50251897 (SEQ ID NO:1051), gi|5107149 (SEQ ID NO:1052), gi|50928231 (SEQ ID NO:1053), CeresClone:584348 (SEQ ID NO:1055), and gi|5107157 (SEQ ID NO:1056). Other homologs and/or orthologs of SEQ ID NO:1042 include Public GI no. 5107082 (SEQ ID NO:1044), Ceres CLONE ID no. 946808 (SEQ ID NO:1046), Ceres CLONE ID no. 617980 (SEQ ID NO:1049), and Ceres CLONE ID no. 714267 (SEQ ID NO:1054).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:1043-1056 or the consensus sequence set forth in FIG. 102.

A regulatory protein can contain a LEA_{—}2 domain characteristic of a late embryogenesis abundant polypeptide. Different types of LEA polypeptides are expressed at different stages of late embryogenesis in higher plant seed embryos and under conditions of dehydration stress. The LEA_{—}2 family represents a group of LEA proteins that appear to be distinct from those in LEA_{—}4. SEQ ID NO:93 sets forth the amino acid sequence of a DNA clone, identified herein as cDNA ID 23819377 (SEQ ID NO:92), that is predicted to encode a polypeptide containing a LEA_{—}2 domain.

A regulatory protein can comprise the amino acid sequence set forth in SEQ ID NO:93. Alternatively, a regulatory protein can be a homolog, ortholog, or variant of the polypeptide having the amino acid sequence set forth in SEQ ID NO:93. For example, a regulatory protein can have an amino acid sequence with at least 40% sequence identity, e.g., 40%, 45%, 50%, 55%, 60%, 65%, 70%, 75%, 80%, 85%, 90%, 95%, 97%, 98%, or 99% sequence identity, to the amino acid sequence set forth in SEQ ID NO:93.

A regulatory protein can contain a C1_{—}2 domain and a C1_{—}3 domain. The C1_{—}2 domain is rich in cysteines and histidines. The pattern of conservation is similar to that found in the C1_{—}1 domain. Therefore, the C1_{—}2 domain has been designated DC1 for divergent C1 domain. The C1_{—}2 domain probably also binds two zinc ions and has been observed to bind to molecules such as diacylglycerol. C1_{—}2 domains are found in plant polypeptides. Like the C1_{—}2 domain, the C1_{—}3 domain also exhibits a pattern of conservation similar that found in C1_{—}1. SEQ ID NO:828 sets forth the amino acid sequence of a DNA clone, identified herein as cDNA ID 23523867 (SEQ ID NO:827), that is predicted to encode a polypeptide containing a C1_{—}2 domain and a C1_{—}3 domain.

A regulatory protein can comprise the amino acid sequence set forth in SEQ ID NO:828. Alternatively, a regulatory protein can be a homolog, ortholog, or variant of the polypeptide having the amino acid sequence set forth in SEQ ID NO:828. For example, a regulatory protein can have an amino acid sequence with at least 20% sequence identity, e.g., 20%, 25%, 30%, 35%, 40%, 45%, 50%, 55%, 60%, 65%, 70%, 75%, 80%, 85%, 90%, 95%, 97%, 98%, or 99% sequence identity, to the amino acid sequence set forth in SEQ ID NO:828.

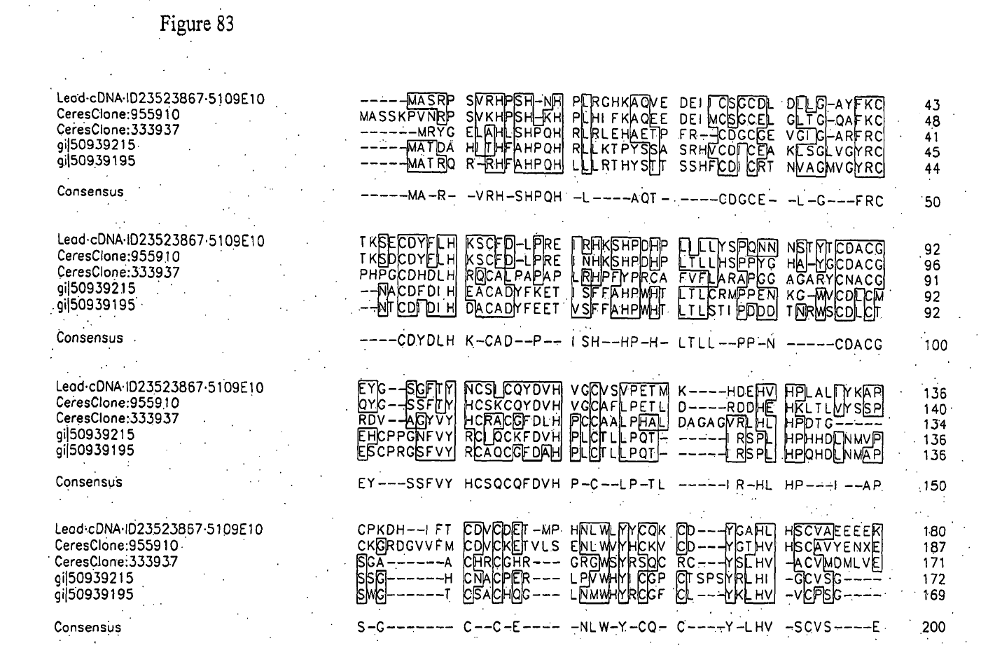

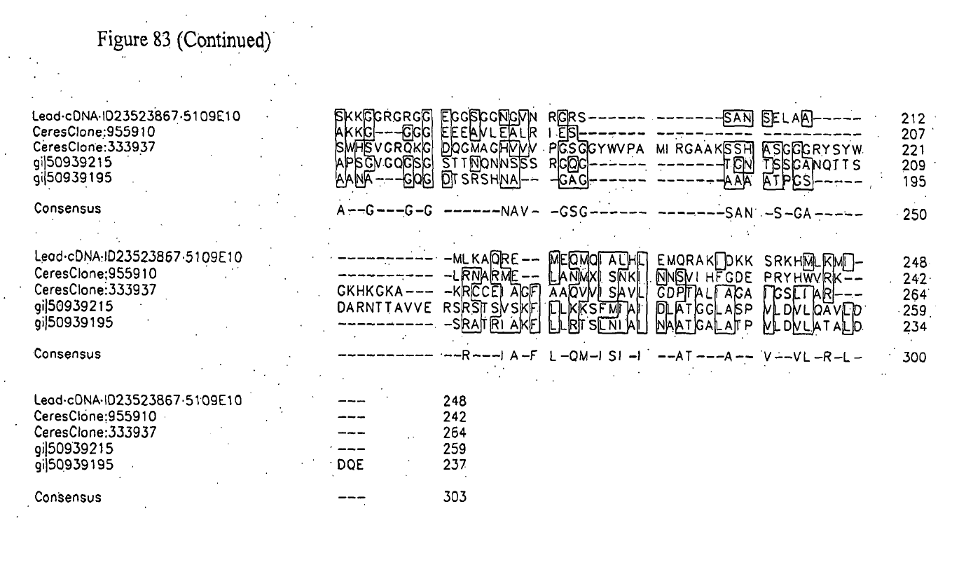

Amino acid sequences of homologs and/or orthologs of the polypeptide having the amino acid sequence set forth in SEQ ID NO:828 are provided in FIG. 83. FIG. 83 also includes a consensus amino acid sequence determined by aligning homologous and/or orthologous amino acid sequences with the amino acid sequence set forth in SEQ ID NO:828.

For example, the alignment in FIG. 83 provides the amino acid sequences of cDNA ID 23523867 (5109E10; SEQ ID NO:828), CeresClone:955910 (SEQ ID NO:829), gi|50939215 (SEQ ID NO:830), gi|50939195 (SEQ ID NO:831), and CeresClone:333937 (SEQ ID NO:832).

In some cases, a regulatory protein can include a polypeptide having at least 80% sequence identity, e.g., 80%, 85%, 90%, 93%, 95%, 97%, 98%, or 99% sequence identity, to an amino acid sequence corresponding to any of SEQ ID NOs:829-832 or the consensus sequence set forth in FIG. 83.

A regulatory protein can have a domain, such as a DUF952 or DUF1313 domain, that is characteristic of a hypothetical polypeptide. The DUF952 family consists of several hypothetical bacterial and plant proteins of unknown function. The DUF1313 family consists of several hypothetical plant proteins of around 100 residues in length. SEQ ID NO:1394 sets forth the amino acid sequence of a DNA clone, identified herein as cDNA ID 23368554 (SEQ ID NO:1393), that is predicted to encode a polypeptide containing a DUF952 domain. SEQ ID NO:1440 sets forth the amino acid sequence of a DNA clone, identified herein as cDNA ID 23814706 (SEQ ID NO:1439), that is predicted to encode a polypeptide containing a DUF1313 domain.

A regulatory protein can comprise the amino acid sequence set forth in SEQ ID NO:1394 or SEQ ID NO:1440. Alternatively, a regulatory protein can be a homolog, ortholog, or variant of the polypeptide having the amino acid sequence set forth in SEQ ID NO:1394 or SEQ ID NO:1440. For example, a regulatory protein can have an amino acid sequence with at least 95% sequence identity, e.g., 96%, 97%, 98%, or 99% sequence identity, to the amino acid sequence set forth in SEQ ID NO:1394 or SEQ ID NO:1440. SEQ ID NO:200, SEQ ID NO:205, SEQ ID NO:225, SEQ ID NO:490, SEQ ID NO:632, SEQ ID NO:639, SEQ ID NO:703, SEQ ID NO:869, SEQ ID NO:871, SEQ ID NO:906, SEQ ID NO:1212, SEQ ID NO:1248, SEQ ID NO:1374, SEQ ID NO:1380, SEQ ID NO:1401, SEQ ID NO:1413, SEQ ID NO:1421, and SEQ ID NO:1452 set forth the amino acid sequences of DNA clones, identified herein as cDNA ID 13610509 (SEQ ID NO:199), cDNA ID 23503364 (SEQ ID NO:204), cDNA ID 23544026 (SEQ ID NO:224), cDNA ID 23357171 (SEQ ID NO:489), cDNA ID 24375036 (SEQ ID NO:631), cDNA ID 23544992 (SEQ ID NO:638), cDNA ID 23740916 (SEQ ID NO:702), cDNA ID 23543586 (SEQ ID NO:868), cDNA ID 4950532 (SEQ ID NO:870), cDNA ID 23557650 (SEQ ID NO:905), cDNA ID 23699071 (SEQ ID NO:1211), cDNA ID 23697027 (SEQ ID NO:1247), cDNA ID 23428062 (SEQ ID NO:1373), cDNA ID 1823190 (SEQ ID NO:1379), cDNA ID 23368864 (SEQ ID NO:1400), cDNA ID 23374628 (SEQ ID NO:1412), cDNA ID 23509990 (SEQ ID NO:1420), and cDNA ID 2706717 (SEQ ID NO:1451), respectively, each of which is predicted to encode a polypeptide that does not have homology to an existing protein family based on Pfam analysis.

A regulatory protein can comprise the amino acid sequence set forth in SEQ ID NO:200, SEQ ID NO:205, SEQ ID NO:225, SEQ ID NO:490, SEQ ID NO:632, SEQ ID NO:639, SEQ ID NO:703, SEQ ID NO:869, SEQ ID NO:871, SEQ ID NO:906, SEQ ID NO:1212, SEQ ID NO:1248, SEQ ID NO:1374, SEQ ID NO:1380, SEQ ID NO:1401, SEQ ID NO:1413, SEQ ID NO:1421, or SEQ ID NO:1452. Alternatively, a regulatory protein can be a homolog, ortholog, or variant of the polypeptide having the amino acid sequence set forth in SEQ ID NO:200, SEQ ID NO:205, SEQ ID NO:225, SEQ ID NO:490, SEQ ID NO:632, SEQ ID NO:639, SEQ ID NO:703, SEQ ID NO:869, SEQ ID NO:871, SEQ ID NO:906, SEQ ID NO:1212, SEQ ID NO:1248, SEQ ID NO:1374, SEQ ID NO:1380, SEQ ID NO:1401, SEQ ID NO:1413, SEQ ID NO:1421, or SEQ ID NO:1452. For example, a regulatory protein can have an amino acid sequence with at least 95% sequence identity, e.g., 96%, 97%, 98%, or 99% sequence identity, to the amino acid sequence set forth in SEQ ID NO:200, SEQ ID NO:205, SEQ ID NO:225, SEQ ID NO:490, SEQ ID NO:632, SEQ ID NO:639, SEQ ID NO:703, SEQ ID NO:869, SEQ ID NO:871, SEQ ID NO:906, SEQ ID NO:1212, SEQ ID NO:1248, SEQ ID NO:1374, SEQ ID NO:1380, SEQ ID NO:1401, SEQ ID NO:1413, SEQ. ID NO:1421, or SEQ ID NO:1452.

A regulatory protein encoded by a recombinant nucleic acid can be a native regulatory protein, i.e., one or more additional copies of the coding sequence for a regulatory protein that is naturally present in the cell. Alternatively, a regulatory protein can be heterologous to the cell, e.g., a transgenic Papaveraceae plant can contain the coding sequence for a transcription factor polypeptide from a Catharanthus plant.

A regulatory protein can include additional amino acids that are not involved in modulating gene expression, and thus can be longer than would otherwise be the case. For example, a regulatory protein can include an amino acid sequence that functions as a reporter. Such a regulatory protein can be a fusion protein in which a green fluorescent protein (GFP) polypeptide is fused to, e.g., SEQ ID NO:80, or in which a yellow fluorescent protein (YFP) polypeptide is fused to, e.g., SEQ ID NO:144. In some embodiments, a regulatory protein includes a purification tag, a chloroplast transit peptide, a mitochondrial transit peptide, or a leader sequence added to the amino or carboxyl terminus.

Regulatory protein candidates suitable for use in the invention can be identified by analysis of nucleotide and polypeptide sequence alignments. For example, performing a query on a database of nucleotide or polypeptide sequences can identify homologs and/or orthologs of regulatory proteins. Sequence analysis can involve BLAST, Reciprocal BLAST, or PSI-BLAST analysis of nonredundant databases using known regulatory protein amino acid sequences. Those polypeptides in the database that have greater than 40% sequence identity can be identified as candidates for further evaluation for suitability as regulatory proteins. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains suspected of being present in regulatory proteins, e.g., conserved functional domains.

The identification of conserved regions in a template or subject polypeptide can facilitate production of variants of regulatory proteins. Conserved regions can be identified by locating a region within the primary amino acid sequence of a template polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains at sanger.ac.uk/Pfam and genome.wustl.edu/Pfam. A description of the information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999).

Conserved regions also can be determined by aligning sequences of the same or related polypeptides from closely related species. Closely related species preferably are from the same family. In some embodiments, alignment of sequences from two different species is adequate. For example, sequences from Arabidopsis and Zea mays can be used to identify one or more conserved regions.

Typically, polypeptides that exhibit at least about 40% amino acid sequence identity are useful to identify conserved regions. Conserved regions of related polypeptides can exhibit at least 45% amino acid sequence identity, e.g., at least 50%, at least 60%, at least 70%, at least 80%, or at least 90% amino acid sequence identity. In some embodiments, a conserved region of target and template polypeptides exhibit at least 92%, 94%, 96%, 98%, or 99% amino acid sequence identity. Amino acid sequence identity can be deduced from amino acid or nucleotide sequences. In certain cases, highly conserved domains have been identified within regulatory proteins. These conserved regions can be useful in identifying functionally similar (orthologous) regulatory proteins.

In some instances, suitable regulatory proteins can be synthesized on the basis of consensus functional domains and/or conserved regions in polypeptides that are homologous regulatory proteins. Domains are groups of substantially contiguous amino acids in a polypeptide that can be used to characterize protein families and/or parts of proteins. Such domains have a “fingerprint” or “signature” that can comprise conserved (1) primary sequence, (2) secondary structure, and/or (3) three-dimensional conformation. Generally, domains are correlated with specific in vitro and/or in vivo activities. A domain can have a length of from 10 amino acids to 400 amino acids, e.g., 10 to 50 amino acids, or 25 to 100 amino acids, or 35 to 65 amino acids, or 35 to 55 amino acids, or 45 to 60 amino acids, or 200 to 300 amino acids, or 300 to 400 amino acids.

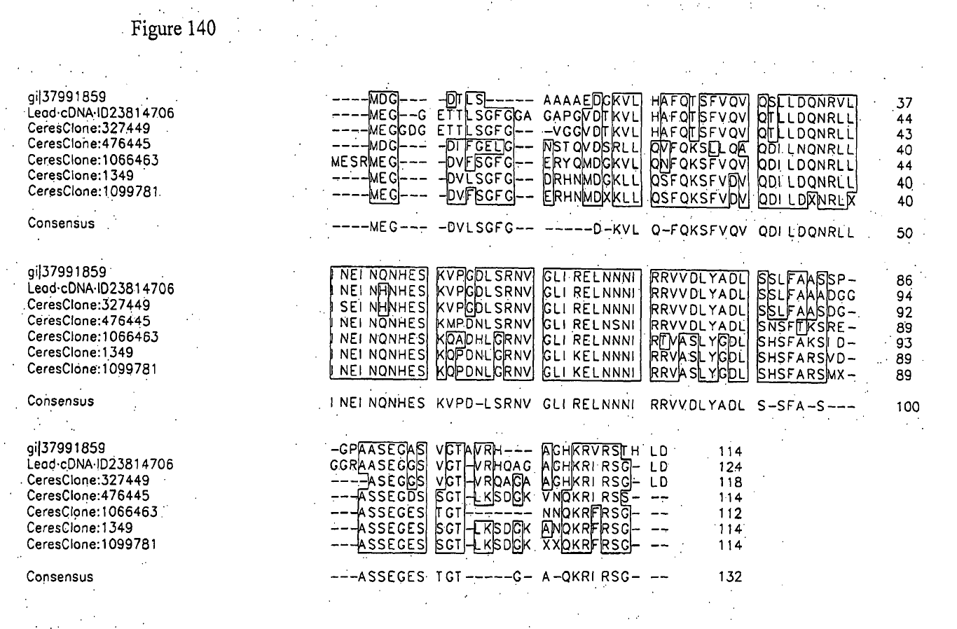

Representative homologs and/or orthologs of regulatory proteins are shown in FIGS. 1-140. Each Figure represents an alignment of the amino acid sequence of a regulatory protein with the amino acid sequences of corresponding homologs and/or orthologs. Amino acid sequences of regulatory proteins and their corresponding homologs and/or orthologs have been aligned to identify conserved amino acids and to determine consensus sequences that contain frequently occurring amino acid residues at particular positions in the aligned sequences, as shown in FIGS. 1-140. A dash in an aligned sequence represents a gap, i.e., a lack of an amino acid at that position. Identical amino acids or conserved amino acid substitutions among aligned sequences are identified by boxes.

Each consensus sequence is comprised of conserved regions. Each conserved region contains a sequence of contiguous amino acid residues. A dash in a consensus sequence indicates that the consensus sequence either lacks an amino acid at that position or includes an amino acid at that position. If an amino acid is present, the residue at that position corresponds to one found in any aligned sequence at that position.

Useful polypeptides can be constructed based on the consensus sequence in any of FIGS. 1-140. Such a polypeptide includes the conserved regions in the selected consensus sequence, arranged in the order depicted in the Figure from amino-terminal end to carboxy-terminal end. Such a polypeptide may also include zero, one, or more than one amino acid in positions marked by dashes. When no amino acids are present at positions marked by dashes, the length of such a polypeptide is the sum of the amino acid residues in all conserved regions. When amino acids are present at all positions marked by dashes, such a polypeptide has a length that is the sum of the amino acid residues in all conserved regions and all dashes.

A conserved domain in certain cases may be 1) a localization domain, 2) an activation domain, 3) a repression domain, 4) an oligomerization domain or 5) a DNA binding domain. Consensus domains and conserved regions can be identified by homologous polypeptide sequence analysis as described above. The suitability of polypeptides for use as regulatory proteins can be evaluated by functional complementation studies.

Alternatively, a regulatory protein can be a fragment of a naturally occurring regulatory protein. In certain cases, such as transcription factor regulatory proteins, a fragment can comprise the DNA-binding and transcription-regulating domains of the naturally occurring regulatory protein.

Additional information on regulatory protein domains is provided below.

### DNA Binding Domain

A regulatory protein can include a domain, termed a DNA binding domain, which binds to a recognized site on DNA. A DNA binding domain of a regulatory protein can bind to one or more specific cis-responsive promoter motifs described herein. The typical result is modulation of transcription from a transcriptional start site associated with and operably linked to the cis-responsive motif. In some embodiments, binding of a DNA binding domain to a cis-responsive motif in planta involves other cellular components, which can be supplied by the plant.

### Transactivation Domain

A regulatory protein can have discrete DNA binding and transactivation domains. Typically, transactivation domains bring proteins of the cellular transcription and translation machinery into contact with the transcription start site to initiate transcription. A transactivation domain of a regulatory protein can be synthetic or can be naturally-occurring. An example of a transactivation domain is the transactivation domain of a maize transcription factor C polypeptide.

### Oligomerization Sequences

In some embodiments, a regulatory protein comprises oligomerization sequences. In some instances oligomerization is required for a ligand/regulatory protein complex or protein/protein complex to bind to a recognized DNA site. Oligomerization sequences can permit a regulatory protein to produce either homo- or heterodimers. Several motifs or domains in the amino acid sequence of a regulatory protein can influence heterodimerization or homodimerization of a given regulatory protein.

In some embodiments, transgenic plants also include a recombinant coactivator polypeptide that can interact with a regulatory protein to mediate the regulatory protein's effect on transcription of an endogenous gene. Such polypeptides include chaperonins.

In some embodiments, a recombinant coactivator polypeptide is a chimera of a non-plant coactivator polypeptide and a plant coactivator polypeptide. Thus, in some embodiments, a regulatory protein described herein binds as a heterodimer to a promoter motif. In such embodiments, plants and plant cells contain a coding sequence for a second or other regulatory protein as a dimerization or multimerization partner, in addition to the coding sequence for the first regulatory protein.

### Nucleic Acids

A nucleic acid can comprise a coding sequence that encodes any of the regulatory proteins as set forth in SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NOs:200-203, SEQ ID NOs:205-209, SEQ ID NOs:211-214, SEQ ID NOs:216-223, SEQ ID NOs:225-227, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NOs:632-635, SEQ ID NO:637, SEQ ID NOs:639-646, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NOs:703-709, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-919, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:1119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NOs:1212-1218, SEQ ID NOs:1220-1224, SEQ ID NOs:1226-1241, SEQ ID NOs:1243-1246, SEQ ID NOs:1248-1253, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NOs:1394-1399, SEQ ID NOs:1401-1402, SEQ ID NOs:1404-1411, SEQ ID NOs:1413-1419, SEQ ID NO:1421, SEQ ID NOs:1423-1427, SEQ ID NOs:1429-1438, SEQ ID NOs:1440-1450, SEQ ID NO:1452, SEQ ID NOs:1476-1484, and the consensus sequences set forth in FIGS. 1-140. In some cases, a recombinant nucleic acid construct can include a nucleic acid comprising less than the full-length coding sequence of a regulatory protein. In some cases, a recombinant nucleic acid construct can include a nucleic acid comprising a coding sequence, a gene, or a fragment of a coding sequence or gene in an antisense orientation so that the antisense strand of RNA is transcribed.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given regulatory protein can be modified such that optimal expression in a particular plant species is obtained, using appropriate codon bias tables for that species.

A nucleic acid also can comprise a nucleotide sequence corresponding to any of the regulatory regions as set forth in SEQ ID NOs:1-78 and SEQ ID NOs:1453-1475. In some cases, a nucleic acid can comprise a nucleotide sequence corresponding to any of the regulatory regions as set forth in SEQ ID NOs:1-78 and SEQ ID NOs:1453-1475 and a coding sequence that encodes any of the regulatory proteins as set forth in SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NOs:200-203, SEQ ID NOs:205-209, SEQ ID NOs:211-214, SEQ ID NOs:216-223, SEQ ID NOs:225-227, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NOs:632-635, SEQ ID NO:637, SEQ ID NOs:639-646, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NOs:703-709, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-919, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:1119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NOs:1212-1218, SEQ ID NOs:1220-1224, SEQ ID NOs:1226-1241, SEQ ID NOs:1243-1246, SEQ ID NOs:1248-1253, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NOs:1394-1399, SEQ ID NOs:1401-1402, SEQ ID NOs:1404-1411, SEQ ID NOs:1413-1419, SEQ ID NO:1421, SEQ ID NOs:1423-1427, SEQ ID NOs:1429-1438, SEQ ID NOs:1440-1450, SEQ ID NO:1452, SEQ ID NOs:1476-1484, and the consensus sequences set forth in FIGS. 1-140.

The terms “nucleic acid” and “polynucleotide” are used interchangeably herein, and refer both to RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

An isolated nucleic acid can be, for example, a naturally-occurring DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Isolated nucleic acid molecules can be produced by standard techniques. For example, polymerase chain reaction (PCR) techniques can be used to obtain an isolated nucleic acid containing a nucleotide sequence described herein. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring DNA.

As used herein, the term “percent sequence identity” refers to the degree of identity between any given query sequence and a subject sequence. A subject sequence typically has a length that is more than 80%, e.g., more than 82%, 85%, 87%, 89%, 90%, 93%, 95%, 97%, 99%, 100%, 105%, 110%, 115%, or 120%, of the length of the query sequence. A query nucleic acid or amino acid sequence is aligned to one or more subject nucleic acid or amino acid sequences using the computer program ClustalW (version 1.83, default parameters), which allows alignments of nucleic acid or protein sequences to be carried out across their entire length (global alignment). Chenna et al., Nucleic Acids Res., 31(13):3497-500 (2003).

ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uk/clustalw).

To determine a percent identity between a query sequence and a subject sequence, ClustalW divides the number of identities in the best alignment by the number of residues compared (gap positions are excluded), and multiplies the result by 100. The output is the percent identity of the subject sequence with respect to the query sequence. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

The term “exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell under consideration. For example, a transgenic plant containing an exogenous nucleic acid can be the progeny of a cross between a stably transformed plant and a non-transgenic plant. Such progeny are considered to contain the exogenous nucleic acid.

Similarly, a regulatory protein can be endogenous or exogenous to a particular plant or plant cell. Exogenous regulatory proteins, therefore, can include proteins that are native to a plant or plant cell, but that are expressed in a plant cell via a recombinant nucleic acid construct, e.g., a California poppy plant transformed with a recombinant nucleic acid construct encoding a California poppy transcription factor.

Likewise, a regulatory region can be exogenous or endogenous to a plant or plant cell. An exogenous regulatory region is a regulatory region that is part of a recombinant nucleic acid construct, or is not in its natural environment. For example, a Nicotiana promoter present on a recombinant nucleic acid construct is an exogenous regulatory region when a Nicotiana plant cell is transformed with the construct.

A transgenic plant or plant cell in which the amount and/or rate of biosynthesis of one or more sequences of interest is modulated includes at least one recombinant nucleic acid construct, e.g., a nucleic acid construct comprising a nucleic acid encoding a regulatory protein or a nucleic acid construct comprising a regulatory region as described herein. In certain cases, more than one recombinant nucleic acid construct can be included (e.g., two, three, four, five, six, or more recombinant nucleic acid constructs). For example, two recombinant nucleic acid constructs can be included, where one construct includes a nucleic acid encoding one regulatory protein, and another construct includes a nucleic acid encoding a second regulatory protein. Alternatively, one construct can include a nucleic acid encoding one regulatory protein, while another includes a regulatory region. In other cases, a plant cell can include a recombinant nucleic acid construct comprising a nucleic acid encoding a regulatory protein and further comprising a regulatory region that associates with the regulatory protein. In such cases, additional recombinant nucleic acid constructs can also be included in the plant cell, e.g., containing additional regulatory proteins and/or regulatory regions.

Vectors containing nucleic acids such as those described herein also are provided. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes a regulatory region. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The vectors provided herein also can include, for example, origins of replication, scaffold attachment regions (SARs), and/or markers. A marker gene can confer a selectable phenotype on a plant cell. For example, a marker can confer biocide resistance, such as resistance to an antibiotic (e.g., kanamycin, G418, bleomycin, or hygromycin), or an herbicide (e.g., chlorosulfuron or phosphinothricin). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

As described herein, plant cells can be transformed with a recombinant nucleic acid construct to express a polypeptide of interest. The polypeptide can then be extracted and purified using techniques known to those having ordinary skill in the art.

### Regulatory Regions

Particular regulatory regions were examined for their ability to associate with regulatory proteins described herein. The sequences of these regulatory regions are set forth in SEQ ID NOs:1453-1468. These regulatory regions were initially chosen for investigation because they were thought to be regulatory regions involved in alkaloid biosynthetic pathways in plants such as Arabidopsis, California poppy, Papaver somniferum, and Catharanthus. Using the methods described herein, regulatory proteins that can associate with some of these regulatory regions were identified, and such associations are listed in Table 4 (under Example 5 below). In turn, knowledge of a regulatory protein-regulatory region association facilitates the modulation of expression of sequences of interest that are operably linked to a given regulatory region by the associated regulatory protein. The regulatory protein associated with the regulatory region operably linked to the sequence of interest is itself operably linked to a regulatory region. The amount and specificity of expression of a regulatory protein can be modulated by selecting an appropriate regulatory region to direct expression of the regulatory protein. For example, a regulatory protein can be broadly expressed under the direction of a promoter such as a CaMV 35S promoter. Once expressed, the regulatory protein can directly or indirectly affect expression of a sequence of interest operably linked to another regulatory region, which is associated with the regulatory protein. In some cases, a regulatory protein can be expressed under the direction of a cell type- or tissue-preferential promoter, such as a cell type- or tissue-preferential promoter described below. In some embodiments, a regulatory region useful in the methods described herein has 80% or greater, e.g., 85%, 90%, 95%, 97%, 98%, 99%, or 100%, sequence identity to a regulatory region set forth in SEQ ID NOs:1453-1468.

The methods described herein can also be used to identify new regulatory region-regulatory protein association pairs. For example, an ortholog to a given regulatory protein is expected to associate with the associated regulatory region for that regulatory protein.

It should be noted that for a given regulatory protein listed in Table 4 (under Example 5 below), a regulatory region construct that includes one or more regulatory regions is set forth. A regulatory protein is expected to associate with either one or both such regulatory regions. Similarly, FIGS. 1-140 provide ortholog/homolog sequences and consensus sequences for corresponding regulatory proteins. It is contemplated that each such ortholog/homolog sequence and each polypeptide sequence that corresponds to the consensus sequence of the regulatory protein would also associate with the regulatory regions associated with the given regulatory protein as set forth in Table 4 (under Example 5 below).

The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

As used herein, the term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site, or about 2,000 nucleotides upstream of the transcription start site. A promoter typically comprises at least a core (basal) promoter. A promoter also may include at least one control element, such as an enhancer sequence, an upstream element or an upstream activation region (UAR). For example, a suitable enhancer is cis-regulatory element (−212 to −154) from the upstream region of the octopine synthase (ocs) gene. Fromm et al., The Plant Cell, 1:977-984 (1989). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Some suitable promoters initiate transcription only, or predominantly, in certain cell types. For example, a promoter that is active predominantly in a reproductive tissue (e.g., fruit, ovule, pollen, pistils, female gametophyte, egg cell, central cell, nucellus, suspensor, synergid cell, flowers, embryonic tissue, embryo sac, embryo, zygote, endosperm, integument, or seed coat) can be used. Thus, as used herein a cell type- or tissue-preferential promoter is one that drives expression preferentially in the target tissue, but may also lead to some expression in other cell types or tissues as well. Methods for identifying and characterizing promoter regions in plant genomic DNA include, for example, those described in the following references: Jordano et al., Plant Cell, 1:855-866 (1989); Bustos et al., Plant Cell, 1:839-854 (1989); Green et al., EMBO J., 7:4035-4044 (1988); Meier et al., Plant Cell, 3:309-316 (1991); and Zhang et al., Plant Physiology, 110:1069-1079 (1996).

Examples of various classes of promoters are described below. Some of the promoters indicated below are described in more detail in U.S. Patent Application Ser. Nos. 60/505,689; 60/518,075; 60/544,771; 60/558,869; 60/583,691; 60/619,181; 60/637,140; 10/950,321; 10/957,569; 11/058,689; 11/172,703; 11/208,308; and PCT/US05/23639. Nucleotide sequences of promoters are set forth in SEQ ID NOs:1-78 and SEQ ID NOs:1453-1475. It will be appreciated that a promoter may meet criteria for one classification based on its activity in one plant species, and yet meet criteria for a different classification based on its activity in another plant species.

Broadly Expressing Promoters

A promoter can be said to be “broadly expressing” when it promotes transcription in many, but not necessarily all, plant tissues. For example, a broadly expressing promoter can promote transcription of an operably linked sequence in one or more of the shoot, shoot tip (apex), and leaves, but weakly or not at all in tissues such as roots or stems. As another example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. Non-limiting examples of broadly expressing promoters that can be included in the nucleic acid constructs provided herein include the p326 (SEQ ID NO:76), YP0144 (SEQ ID NO:55), YP0190 (SEQ ID NO:59), p13879 (SEQ ID NO:75), YP0050 (SEQ ID NO:35), p32449 (SEQ ID NO:77), 21876 (SEQ ID NO:1), YP0158 (SEQ ID NO:57), YP0214 (SEQ ID NO:61), YP0380 (SEQ ID NO:70), PT0848 (SEQ ID NO:26), and PT0633 (SEQ ID NO:7) promoters. Additional examples include the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter, and ubiquitin promoters such as the maize ubiquitin-1 promoter. In some cases, the CaMV 35S promoter is excluded from the category of broadly expressing promoters.

Root Promoters

Root-active promoters confer transcription in root tissue, e.g., root endodermis, root epidermis, or root vascular tissues. In some embodiments, root-active promoters are root-preferential promoters, i.e., confer transcription only or predominantly in root tissue. Root-preferential promoters include the YP0128 (SEQ ID NO:52), YP0275 (SEQ ID NO:63), PT0625 (SEQ ID NO:6), PT0660 (SEQ ID NO:9), PT0683 (SEQ ID NO:14), and PT0758 (SEQ ID NO:22) promoters. Other root-preferential promoters include the PT0613 (SEQ ID NO:5), PT0672 (SEQ ID NO:11), PT0688 (SEQ ID NO:15), and PT0837 (SEQ ID NO:24) promoters, which drive transcription primarily in root tissue and to a lesser extent in ovules and/or seeds. Other examples of root-preferential promoters include the root-specific subdomains of the CaMV 35S promoter (Lam et al., Proc. Natl. Acad. Sci. USA, 86:7890-7894 (1989)), root cell specific promoters reported by Conkling et al., Plant Physiol., 93:1203-1211 (1990), and the tobacco RD2 promoter.

Maturing Endosperm Promoters

In some embodiments, promoters that drive transcription in maturing endosperm can be useful. Transcription from a maturing endosperm promoter typically begins after fertilization and occurs primarily in endosperm tissue during seed development and is typically highest during the cellularization phase. Most suitable are promoters that are active predominantly in maturing endosperm, although promoters that are also active in other tissues can sometimes be used. Non-limiting examples of maturing endosperm promoters that can be included in the nucleic acid constructs provided herein include the napin promoter, the Arcelin-5 promoter, the phaseolin promoter (Bustos et al., Plant Cell, 1(9):839-853 (1989)), the soybean trypsin inhibitor promoter (Riggs et al., Plant Cell, 1(6):609-621 (1989)), the ACP promoter (Baerson et al., Plant Mol. Biol., 22(2):255-267 (1993)), the stearoyl-ACP desaturase promoter (Slocombe et al., Plant Physiol., 104(4):167-176 (1994)), the soybean α subunit of β-conglycinin promoter (Chen et al., Proc. Natl. Acad. Sci. USA, 83:8560-8564 (1986)), the oleosin promoter (Hong et al., Plant Mol. Biol., 34(3):549-555 (1997)), and zein promoters, such as the 15 kD zein promoter, the 16 kD zein promoter, 19 kD zein promoter, 22 kD zein promoter and 27 kD zein promoter. Also suitable are the Osgt-1 promoter from the rice glutelin-1 gene (Zheng et al., Mol. Cell. Biol., 13:5829-5842 (1993)), the beta-amylase promoter, and the barley hordein promoter. Other maturing endosperm promoters include the YP0092 (SEQ ID NO:38), PT0676 (SEQ ID NO:12), and PT0708 (SEQ ID NO:17) promoters.

Ovary Tissue Promoters

Promoters that are active in ovary tissues such as the ovule wall and mesocarp can also be useful, e.g., a polygalacturonidase promoter, the banana TRX promoter, and the melon actin promoter. Examples of promoters that are active primarily in ovules include YP0007 (SEQ ID NO:30), YP0111 (SEQ ID NO:46), YP0092 (SEQ ID NO:38), YP0103 (SEQ ID NO:43), YP0028 (SEQ ID NO:33), YP0121 (SEQ ID NO:51), YP0008 (SEQ ID NO:31), YP0039 (SEQ ID NO:34), YP0115 (SEQ ID NO:47), YP0119 (SEQ ID NO:49), YP0120 (SEQ ID NO:50), and YP0374 (SEQ ID NO:68).

Embryo Sac/Early Endosperm Promoters

To achieve expression in embryo sac/early endosperm, regulatory regions can be used that are active in polar nuclei and/or the central cell, or in precursors to polar nuclei, but not in egg cells or precursors to egg cells. Most suitable are promoters that drive expression only or predominantly in polar nuclei or precursors thereto and/or the central cell. A pattern of transcription that extends from polar nuclei into early endosperm development can also be found with embryo sac/early endosperm-preferential promoters, although transcription typically decreases significantly in later endosperm development during and after the cellularization phase. Expression in the zygote or developing embryo typically is not present with embryo sac/early endosperm promoters.

Promoters that may be suitable include those derived from the following genes: Arabidopsis viviparous-1 (see, GenBank No. U93215); Arabidopsis atmycl (see, Urao (1996) Plant Mol. Biol., 32:571-57; Conceicao (1994) Plant, 5:493-505); Arabidopsis FIE (GenBank No. AF129516); Arabidopsis MEA; Arabidopsis FIS2 (GenBank No. AF096096); and FIE 1.1 (U.S. Pat. No. 6,906,244). Other promoters that may be suitable include those derived from the following genes: maize MAC1 (see, Sheridan (1996) Genetics, 142:1009-1020); maize Cat3 (see, GenBank No. L05934; Abler (1993) Plant Mol. Biol., 22:10131-1038). Other promoters include the following Arabidopsis promoters: YP0039 (SEQ ID NO:34), YP0101 (SEQ ID NO:41), YP0102 (SEQ ID NO:42), YP0110 (SEQ ID NO:45), YP0117 (SEQ ID NO:48), YP019 (SEQ ID NO:49), YP0137 (SEQ ID NO:53), DME, YP0285 (SEQ ID NO:64), and YP0212 (SEQ ID NO:60). Other promoters that may be useful include the following rice promoters: p530c10, pOsFIE2-2, pOsMEA, pOsYp102, and pOsYp285.

Embryo Promoters

Regulatory regions that preferentially drive transcription in zygotic cells following fertilization can provide embryo-preferential expression. Most suitable are promoters that preferentially drive transcription in early stage embryos prior to the heart stage, but expression in late stage and maturing embryos is also suitable. Embryo-preferential promoters include the barley lipid transfer protein (Ltp1) promoter (Plant Cell Rep (2001) 20:647-654), YP0097 (SEQ ID NO:40), YP0107 (SEQ ID NO:44), YP0088 (SEQ ID NO:37), YP0143 (SEQ ID NO:54), YP0156 (SEQ ID NO:56), PT0650 (SEQ ID NO:8), PT0695 (SEQ ID NO:16), PT0723 (SEQ ID NO:19), PT0838 (SEQ ID NO:25), PT0879 (SEQ ID NO:28), and PT0740 (SEQ ID NO:20).

Photosynthetic Tissue Promoters

Promoters active in photosynthetic tissue confer transcription in green tissues such as leaves and stems. Most suitable are promoters that drive expression only or predominantly in such tissues. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol., 35:773-778 (1994)), the Cab-1 promoter from wheat (Fejes et al., Plant Mol. Biol., 15:921-932 (1990)), the CAB-1 promoter from spinach (Lubberstedt et al., Plant Physiol., 104:997-1006 (1994)), the cab1R promoter from rice (Luan et al., Plant Cell, 4:971-981 (1992)), the pyruvate orthophosphate dikinase (PPDK) promoter from corn (Matsuoka et al., Proc. Natl. Acad. Sci. USA, 90:9586-9590 (1993)), the tobacco Lhcb1*2 promoter (Cerdan et al., Plant Mol. Biol., 33:245-255 (1997)), the Arabidopsis thaliana SUC2 sucrose-H+ symporter promoter (Truernit et al., Planta, 196:564-570 (1995)), and thylakoid membrane protein promoters from spinach (psaD, psaF, psaE, PC, FNR, atpC, atpD, cab, rbcS). Other photosynthetic tissue promoters include PT0535 (SEQ ID NO:3), PT0668 (SEQ ID NO:2), PT0886 (SEQ ID NO:29), YP0144 (SEQ ID NO:55), YP0380 (SEQ ID NO:70), and PT0585 (SEQ ID NO:4).

Vascular Tissue Promoters

Examples of promoters that have high or preferential activity in vascular bundles include YP0087 (SEQ ID NO:1469), YP0093 (SEQ ID NO:1470), YP0108 (SEQ ID NO:1471), YP0022 (SEQ ID NO:1472), and YP0080 (SEQ ID NO:1473). Other vascular tissue-preferential promoters include the glycine-rich cell wall protein GRP 1.8 promoter (Keller and Baumgartner, Plant Cell, 3(10):1051-1061 (1991)), the Commelina yellow mottle virus (CoYMV) promoter (Medberry et al., Plant Cell, 4(2):185-192 (1992)), and the rice tungro bacilliform virus (RTBV) promoter (Dai et al., Proc. Natl. Acad. Sci. USA, 101(2):687-692 (2004)).

Poppy Capsule Promoters

Examples of promoters that have high or preferential activity in siliques/fruits, which are botanically equivalent to capsules in opium poppy, include PT0565 (SEQ ID NO:1474) and YP0015 (SEQ ID NO:1475).

Inducible Promoters

Inducible promoters confer transcription in response to external stimuli such as chemical agents or environmental stimuli. For example, inducible promoters can confer transcription in response to hormones such as gibberellic acid or ethylene, or in response to light or drought. Examples of drought-inducible promoters include YP0380 (SEQ ID NO:70), PT0848 (SEQ ID NO:26), YP0381 (SEQ ID NO:71), YP0337 (SEQ ID NO:66), PT0633 (SEQ ID NO:7), YP0374 (SEQ ID NO:68), PT0710 (SEQ ID NO:18), YP0356 (SEQ ID NO:67), YP0385 (SEQ ID NO:73), YP0396 (SEQ ID NO:74), YP0388, YP0384 (SEQ ID NO:72), PT0688 (SEQ ID NO:15), YP0286 (SEQ ID NO:65), YP0377 (SEQ ID NO:69), PD1367 (SEQ ID NO:78), PD0901, and PD0898. Nitrogen-inducible promoters include PT0863 (SEQ ID NO:27), PT0829 (SEQ ID NO:23), PT0665 (SEQ ID NO:10), and PT0886 (SEQ ID NO:29).

Basal Promoters

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

Other Promoters

Other classes of promoters include, but are not limited to, leaf-preferential, stem/shoot-preferential, callus-preferential, guard cell-preferential, such as PT0678 (SEQ ID NO:13), and senescence-preferential promoters. Promoters designated YP0086 (SEQ ID NO:36), YP0188 (SEQ ID NO:58), YP0263 (SEQ ID NO:62), PT0758 (SEQ ID NO:22), PT0743 (SEQ ID NO:21), PT0829 (SEQ ID NO:23), YP0119 (SEQ ID NO:49), and YP0096 (SEQ ID NO:39), as described in the above-referenced patent applications, may also be useful.

Other Regulatory Regions

A 5′ untranslated region (UTR) can be included in nucleic acid constructs described herein. A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

It will be understood that more than one regulatory region may be present in a recombinant polynucleotide, e.g., introns, enhancers, upstream activation regions, transcription terminators, and inducible elements. Thus, more than one regulatory region can be operably linked to the sequence of a polynucleotide encoding a regulatory protein.

Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation.

### Sequences of Interest and Plants and Plant Cells Containing the Same

Plant cells and plants described herein are useful because expression of a sequence of interest can be modulated to achieve a desired amount and/or specificity in expression by selecting an appropriate association of regulatory region and regulatory protein. A sequence of interest operably linked to a regulatory region can encode a polypeptide or can regulate the expression of a polypeptide. In some embodiments, a sequence of interest is transcribed into an anti-sense molecule. In some embodiments, more than one sequence of interest is present in a plant, e.g., two, three, four, five, six, seven, eight, nine, or ten sequences of interest. Each sequence of interest can be present on the same nucleic acid construct in such embodiments. Alternatively, each sequence of interest can be present on separate nucleic acid constructs. The regulatory region operably linked to each sequence of interest can be the same or can be different. In addition, one or more nucleotide sequences encoding a regulatory protein can be included on a nucleic acid construct that is the same as or separate from that containing an associated regulatory region(s) operably linked to a sequence(s) of interest. The regulatory region operably linked to each sequence encoding a regulatory protein can be the same or different.

A sequence of interest that encodes a polypeptide can encode a plant polypeptide, a non-plant polypeptide, e.g., a mammalian polypeptide, a modified polypeptide, a synthetic polypeptide, or a portion of a polypeptide. A sequence of interest can be endogenous, i.e., unmodified by recombinant DNA technology from the sequence and structural relationships that occur in nature and operably linked to the unmodified regulatory region. Alternatively, a sequence of interest can be an exogenous nucleic acid.

Alkaloid Biosynthesis Sequences

In certain cases, a sequence of interest can be an endogenous or exogenous sequence associated with alkaloid biosynthesis. For example, a transgenic plant cell containing a recombinant nucleic acid encoding a regulatory protein can be effective for modulating the amount and/or rate of biosynthesis of one or more alkaloid compounds. Such effects on alkaloid compounds typically occur via modulation of transcription of one or more endogenous or exogenous sequences of interest operably linked to an associated regulatory region, e.g., endogenous sequences involved in alkaloid biosynthesis, such as native enzymes or regulatory proteins in alkaloid biosynthesis pathways, or exogenous sequences involved in alkaloid biosynthesis pathways introduced via a recombinant nucleic acid construct into a plant cell.

In some embodiments, the coding sequence can encode a polypeptide involved in alkaloid biosynthesis, e.g., an enzyme involved in biosynthesis of the alkaloid compounds described herein, or a regulatory protein (such as a transcription factor) involved in the biosynthesis pathways of the alkaloid compounds described herein. Other components that may be present in a sequence of interest include introns, enhancers, upstream activation regions, and inducible elements.

A suitable sequence of interest can encode an enzyme involved in tetrahydrobenzylisoquinoline alkaloid biosynthesis, e.g., selected from the group consisting of those encoding for tyrosine decarboxylase (YDC or TYD; EC 4.1.1.25), norcoclaurine synthase (EC 4.2.1.78), coclaurine N-methyltransferase (EC 2.1.1.140), (R,S)-norcoclaurine 6-O-methyl transferase (NOMT; EC 2.1.1.128), S-adenosyl-L-methionine:3′-hydroxy-N-methylcoclaurine 4′-O-methyltransferase 1 (HMCOMT1; EC 2.1.1.116); S-adenosyl-L-methionine:3′-hydroxy-N-methylcoclaurine 4′-O-methyltransferase 2 (HMCOMT2; EC 2.1.1.116); monophenol monooxygenase (EC1.14.18.1), N-methylcoclaurine 3′-hydroxylase (NMCH EC 1.14.13.71), (R,S)-reticuline 7-O-methyltransferase (ROMT); berbamunine synthase (EC 1.14.21.3), columbamine O-methyltransferase (EC 2.1.1.118), berberine bridge enzyme (BBE; (EC 1.21.3.3), reticuline oxidase (EC 1.21.3.4), dehydro reticulinium ion reductase (EC 1.5.1.27), (RS)-1-benzyl-1,2,3,4-tetrahydroisoquinoline N-methyltransferase (EC 2.1.1.115), (S)-scoulerine oxidase (EC 1.14.21.2), (S)-cheilanthifoline oxidase (EC 1.14.21.1), (S)-tetrahydroprotoberberine N-methyltransferase (EC 2.1.1.122), (S)-canadine synthase (EC 1.14.21.5), tetrahydroberberine oxidase (EC 1.3.3.8), columbamine oxidase (EC 1.21.3.2), and other enzymes, such as protopine-6-monooxygenase, related to the biosynthesis of tetrahydrobenzylisoquinoline alkaloids.

In other cases, a sequence of interest can be an enzyme involved in benzophenanthridine alkaloid biosynthesis, e.g., selected from the group consisting of those encoding for dihydrobenzophenanthridine oxidase (EC 1.5.3.12), dihydrosanguinarine 10-hydroxylase (EC 1.14.13.56), 10-hydroxydihydrosanguinarine 10-O-methyltransferase (EC 2.1.1.119), dihydrochelirubine 12-hydroxylase (EC 1.14.13.57), 12-hydroxydihydrochelirubine 12-O-methyltransferase (EC 2.1.1.120), and other enzymes, including dihydrobenzophenanthridine oxidase and dihydrosanguinarine 10-monooxygenase, related to the biosynthesis of benzophenanthridine alkaloids.

In yet other cases, a sequence is involved in morphinan alkaloid biosynthesis, e.g., selected from the group consisting of salutaridinol 7-O-acetyltransferase (SAT; EC 2.3.1.150), salutaridine synthase (EC 1.14.21.4), salutaridine reductase (EC 1.1.1.248), morphine 6-dehydrogenase (EC 1.1.1.218); and codeinone reductase (CR; EC 1.1.1.247); and other sequences related to the biosynthesis of morphinan/opiate alkaloids.

In other embodiments, a suitable sequence encodes an enzyme involved in purine alkaloid (e.g., xanthines, such as caffeine) biosynthesis such as xanthosine methyltransferase, 7-N-methylxanthine methyltransferase (theobromine synthase), or 3,7-dimethylxanthine methyltransferase (caffeine synthase).

In some embodiments, a suitable sequence encodes an enzyme involved in biosynthesis of indole alkaloids compounds such as tryptophane decarboxylase, strictosidine synthase, strictosidine glycosidase, dehydrogeissosshizine oxidoreductase, polyneuridine aldehyde esterase, sarpagine bridge enzyme, vinorine reductase, vinorine synthase, vinorine hydroxylase, 17-O-acetylajmalan acetylesterase, or norajamaline N-methyl transferase. In other embodiments, a suitable sequence of interest encodes an enzyme involved in biosynthesis of vinblastine, vincristine and compounds derived from them, such as tabersonine 16-hydroxylase, 16-hydroxytabersonine 16-O-methyl transferase, desacetoxyvindoline 4-hydroxylase, or desacetylvindoline O-acetyltransferasesynthase.

In still other embodiments, a suitable sequence encodes an enzyme involved in biosynthesis of pyridine, tropane, and/or pyrrolizidine alkaloids such as arginine decarboxylase, spermidine synthase, ornithine decarboxylase, putrescine N-methyl transferase, tropinone reductase, hyoscyamine 6-beta-hydroxylase, diamine oxidase, and tropinone dehydrogenase.

Other Sequences of Interest

Other sequences of interest can encode a therapeutic polypeptide for use with mammals such as humans, e.g., as set forth in Table 1, below. In certain cases, a sequence of interest can encode an antibody or antibody fragment. An antibody or antibody fragment includes a humanized or chimeric antibody, a single chain Fv antibody fragment, an Fab fragment, and an F(ab)₂ fragment. A chimeric antibody is a molecule in which different portions are derived from different animal species, such as those having a variable region derived from a mouse monoclonal antibody and a human immunoglobulin constant region. Antibody fragments that have a specific binding affinity can be generated by known techniques. Such antibody fragments include, but are not limited to, F(ab′)₂ fragments that can be produced by pepsin digestion of an antibody molecule, and Fab fragments that can be generated by deducing the disulfide bridges of F(ab′)₂ fragments. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques, such as those disclosed in U.S. Pat. No. 4,946,778. U.S. Pat. No. 6,303,341 discloses immunoglobulin receptors. U.S. Pat. No. 6,417,429 discloses immunoglobulin heavy- and light-chain polypeptides.

TABLE 1
Human Therapeutic Proteins

| | | |
|---|---|---|
| Bromelain | Humatrope ® | Proleukin ® |
| Chymopapain | Humulin ® (insulin) | Protropin ® |
| Papain ® | Infergen ® | Recombivax-HB ® |
| Activase ® | Interferon-gamma-1a | Recormon ® |
| Albutein ® | Interleukin-2 | Remicade ® (s-TNF-r) |
| Angiotensin II | Intron ® | ReoPro ® |
| Asparaginase | Leukine ® (GM-CSF) | Retavase ® (TPA) |
| Avonex ® | Nartogastrim ® | Roferon-A ® |
| Betaseron ® | Neumega ® | Pegaspargas |
| BioTropin ® | Neupogen ® | Prandin ® |
| Cerezyme ® | Norditropin ® | Procrit ® |
| Enbrel ® (s-TNF-r) | Novolin ® (insulin) | Filgastrim ® |
| Engerix-B ® | Nutropin ® | Genotropin ® |
| Epogen ® | Oncaspar ® | Geref ® |
| Sargramostrim | Tripedia ® | Trichosanthin |
| TriHIBit ® | Venoglobin-S ® (HIG) | |

A sequence of interest can encode a polypeptide or result in a transcription product anti-sense molecule that confers insect resistance, bacterial disease resistance, fungal disease resistance, viral disease resistance, nematode disease resistance, herbicide resistance, enhanced grain composition or quality, enhanced nutrient composition, nutrient transporter functions, enhanced nutrient utilization, enhanced environmental stress tolerance, reduced mycotoxin contamination, female sterility, a selectable marker phenotype, a screenable marker phenotype, a negative selectable marker phenotype, or altered plant agronomic characteristics. Specific examples include, without limitation, a chitinase coding sequence and a glucan endo-1,3-β-glucosidase coding sequence. In some embodiments, a sequence of interest encodes a bacterial ESPS synthase that confers resistance to glyphosate herbicide or a phosphinothricin acetyl transferase coding sequence that confers resistance to phosphinothricin herbicide.

A sequence of interest can encode a polypeptide involved in the production of industrial or pharmaceutical chemicals, modified and specialty oils, enzymes, or renewable non-foods such as fuels and plastics, vaccines and antibodies. U.S. Pat. No. 5,824,779 discloses phytase-protein-pigmenting concentrate derived from green plant juice. U.S. Pat. No. 5,900,525 discloses animal feed compositions containing phytase derived from transgenic alfalfa. U.S. Pat. No. 6,136,320 discloses vaccines produced in transgenic plants. U.S. Pat. No. 6,255,562 discloses insulin. U.S. Pat. No. 5,958,745 discloses the formation of copolymers of 3-hydroxy butyrate and 3-hydroxy valerate. U.S. Pat. No. 5,824,798 discloses starch synthases. U.S. Pat. No. 6,087,558 discloses the production of proteases in plants. U.S. Pat. No. 6,271,016 discloses an anthranilate synthase gene for tryptophan overproduction in plants.

### Methods of Inhibiting Expression of a Sequence of Interest

The polynucleotides and recombinant vectors described herein can be used to express or inhibit expression of a gene, such as an endogenous gene involved in alkaloid biosynthesis, e.g., to alter alkaloid biosynthetic pathways in a plant species of interest. The term “expression” refers to the process of converting genetic information of a polynucleotide into RNA through transcription, which is catalyzed by an enzyme, RNA polymerase, and into protein, through translation of mRNA on ribosomes. “(Up-regulation” or “activation” refers to regulation that increases the production of expression products (mRNA, polypeptide, or both) relative to basal or native states, while “down-regulation” or “repression” refers to regulation that decreases production of expression products (mRNA, polypeptide, or both) relative to basal or native states.

“Modulated level of gene expression” as used herein refers to a comparison of the level of expression of a transcript of a gene or the amount of its corresponding polypeptide in the presence and absence of a regulatory protein described herein, and refers to a measurable or observable change in the level of expression of a transcript of a gene or the amount of its corresponding polypeptide relative to a control plant or plant cell under the same conditions (e.g., as measured through a suitable assay such as quantitative RT-PCR, a “northern blot,” a “western blot” or through an observable change in phenotype, chemical profile, or metabolic profile). A modulated level of gene expression can include up-regulated or down-regulated expression of a transcript of a gene or polypeptide relative to a control plant or plant cell under the same conditions. Modulated expression levels can occur under different environmental or developmental conditions or in different locations than those exhibited by a plant or plant cell in its native state.

A number of nucleic acid based methods, including antisense RNA, co-suppression, ribozyme directed RNA cleavage, and RNA interference (RNAi) can be used to inhibit protein expression in plants. Antisense technology is one well-known method. In this method, a nucleic acid segment from a gene to be repressed is cloned and operably linked to a promoter so that the antisense strand of RNA is transcribed. The recombinant vector is then transformed into plants, as described above, and the antisense strand of RNA is produced. The nucleic acid segment need not be the entire sequence of the gene to be repressed, but typically will be substantially complementary to at least a portion of the sense strand of the gene to be repressed. Generally, higher homology can be used to compensate for the use of a shorter sequence. Typically, a sequence of at least 30 nucleotides is used, e.g., at least 40, 50, 80, 100, 200, 500 nucleotides or more.

Constructs containing operably linked nucleic acid molecules in the sense orientation can also be used to inhibit the expression of a gene. The transcription product can be similar or identical to the sense coding sequence of a polypeptide of interest. The transcription product can also be unpolyadenylated, lack a 5′ cap structure, or contain an unsplicable intron. Methods of co-suppression using a full-length cDNA as well as a partial cDNA sequence are known in the art. See, e.g., U.S. Pat. No. 5,231,020.

In another method, a nucleic acid can be transcribed into a ribozyme, or catalytic RNA, that affects expression of an mRNA. (See, U.S. Pat. No. 6,423,885). Ribozymes can be designed to specifically pair with virtually any target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contain a 5′-UG-3′ nucleotide sequence. The construction and production of hammerhead ribozymes is known in the art. See, for example, U.S. Pat. No. 5,254,678 and WO 02/46449 and references cited therein. Hammerhead ribozyme sequences can be embedded in a stable RNA such as a transfer RNA (tRNA) to increase cleavage efficiency in vivo. Perriman et al., Proc. Natl. Acad. Sci. USA, 92(13):6175-6179 (1995); de Feyter and Gaudron, Methods in Molecular Biology, Vol. 74, Chapter 43, “Expressing Ribozymes in Plants”, Edited by Turner, P. C., Humana Press Inc., Totowa, N.J. RNA endoribonucleases which have been described, such as the one that occurs naturally in Tetrahymena thermophila, can be useful. See, for example, U.S. Pat. Nos. 4,987,071 and 6,423,885.

RNAi can also be used to inhibit the expression of a gene. For example, a construct can be prepared that includes a sequence that is transcribed into an interfering RNA. Such an RNA can be one that can anneal to itself, e.g., a double stranded RNA having a stem-loop structure. One strand of the stem portion of a double stranded RNA comprises a sequence that is similar or identical to the sense coding sequence of the polypeptide of interest, and that is from about 10 nucleotides to about 2,500 nucleotides in length. The length of the sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA comprises a sequence that is similar or identical to the antisense strand of the coding sequence of the polypeptide of interest, and can have a length that is shorter, the same as, or longer than the corresponding length of the sense sequence. The loop portion of a double stranded RNA can be from 10 nucleotides to 5,000 nucleotides, e.g., from 15 nucleotides to 1,000 nucleotides, from 20 nucleotides to 500 nucleotides, or from 25 nucleotides to 200 nucleotides. The loop portion of the RNA can include an intron. A construct including a sequence that is transcribed into an interfering RNA is transformed into plants as described above. Methods for using RNAi to inhibit the expression of a gene are known to those of skill in the art. See, e.g., U.S. Pat. Nos. 5,034,323; 6,326,527; 6,452,067; 6,573,099; 6,753,139; and 6,777,588. See also WO 97/01952; WO 98/53083; WO 99/32619; WO 98/36083; and U.S. Patent Publications 20030175965, 20030175783, 20040214330, and 20030180945.

In some nucleic-acid based methods for inhibition of gene expression in plants, a suitable nucleic acid can be a nucleic acid analog. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered morpholino ring, or peptide nucleic acids, in which the deoxyphosphate backbone is replaced by a pseudopeptide backbone and the four bases are retained. See, for example, Summerton and Weller, 1997, Antisense Nucleic Acid Drug Dev., 7:187-195; Hyrup et al., Bioorgan. Med. Chem., 4:5-23 (1996). In addition, the deoxyphosphate backbone can be replaced with, for example, a phosphorothioate or phosphorodithioate backbone, a phosphoroamidite, or an alkyl phosphotriester backbone.

### Transgenic Plant Cells and Plants

Provided herein are transgenic plant cells and plants comprising at least one recombinant nucleic acid construct or exogenous nucleic acid. A recombinant nucleic acid construct or exogenous nucleic acid can include a regulatory region as described herein, a nucleic acid encoding a regulatory protein as described herein, or both. In certain cases, a transgenic plant cell or plant comprises at least two recombinant nucleic acid constructs or exogenous nucleic acids, one including a regulatory region, and one including a nucleic acid encoding the associated regulatory protein.

A plant or plant cell used in methods of the invention contains a recombinant nucleic acid construct as described herein. A plant or plant cell can be transformed by having a construct integrated into its genome, i.e., can be stably transformed. Stably transformed cells typically retain the introduced nucleic acid with each cell division. A plant or plant cell can also be transiently transformed such that the construct is not integrated into its genome. Transiently transformed cells typically lose all or some portion of the introduced nucleic acid construct with each cell division such that the introduced nucleic acid cannot be detected in daughter cells after a sufficient number of cell divisions. Both transiently transformed and stably transformed transgenic plants and plant cells can be useful in the methods described herein.

Typically, transgenic plant cells used in methods described herein constitute part or all of a whole plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species or for further selection of other desirable traits. Alternatively, transgenic plants can be propagated vegetatively for those species amenable to such techniques. Progeny includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC₁, F₁BC₂, F₁BC₃, and subsequent generation plants. Seeds produced by a transgenic plant can be grown and then selfed (or outcrossed and selfed) to obtain seeds homozygous for the nucleic acid construct.

Transgenic plant cells growing in suspension culture, or tissue or organ culture, can be useful for extraction of alkaloid compounds. For the purposes of this invention, solid and/or liquid tissue culture techniques can be used. When using solid medium, transgenic plant cells can be placed directly onto the medium or can be placed onto a filter film that is then placed in contact with the medium. When using liquid medium, transgenic plant cells can be placed onto a floatation device, e.g., a porous membrane that contacts the liquid medium. Solid medium typically is made from liquid medium by adding agar. For example, a solid medium can be Murashige and Skoog (MS) medium containing agar and a suitable concentration of an auxin, e.g., 2,4-dichlorophenoxyacetic acid (2,4-D), and a suitable concentration of a cytokinin, e.g., kinetin.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous regulatory protein whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation, e.g., U.S. Pat. Nos. 5,538,880, 5,204,253, 6,329,571 and 6,013,863. If a cell or tissue culture is used as the recipient tissue for transformation, plants can be regenerated from transformed cultures if desired, by techniques known to those skilled in the art. See, e.g., Allen et al., “RNAi-mediated replacement of morphine with the nonnarcotic alkaloid reticuline in opium poppy,” Nature Biotechnology 22(12):1559-1566 (2004); Chitty et al., “Genetic transformation in commercial Tasmanian cultures of opium poppy, Papaver somniferum, and movement of transgenic pollen in the field,” Funct. Plant Biol. 30:1045-1058 (2003); and Park et al., J. Exp. Botany 51(347):1005-1016 (2000).

### Plant Species

The polynucleotides and vectors described herein can be used to transform a number of monocotyledonous and dicotyledonous plants and plant cell systems. A suitable group of plant species includes dicots, such as poppy, safflower, alfalfa, soybean, cotton, coffee, rapeseed (high erucic acid and canola), or sunflower. Also suitable are monocots such as corn, wheat, rye, barley, oat, rice, millet, amaranth or sorghum. Also suitable are vegetable crops or root crops such as lettuce, carrot, onion, broccoli, peas, sweet corn, popcorn, tomato, potato, beans (including kidney beans, lima beans, dry beans, green beans) and the like. Also suitable are fruit crops such as grape, strawberry, pineapple, melon (e.g., watermelon, cantaloupe), peach, pear, apple, cherry, orange, lemon, grapefruit, plum, mango, banana, and palm.

Thus, the methods and compositions described herein can be utilized with dicotyledonous plants belonging to the orders Magniolales, Illiciales, Laurales, Piperales, Aristolochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, Santales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales. Methods described herein can also be utilized with monocotyledonous plants belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchidales, or with plants belonging to Gymnospermae, e.g., Pinales, Ginkgoales, Cycadales and Gnetales.

The invention has use over a broad range of plant species, including species from the genera Allium, Alseodaphne, Anacardium, Arachis, Asparagus, Atropa, Avena, Beilschmiedia, Brassica, Citrus, Citrullus, Capsicum, Catharanthus, Carthamus, Cocculus, Cocos, Coffea, Croton, Cucumis, Cucurbita, Daucus, Duguetia, Elaeis, Eschscholzia, Ficus, Fragaria, Glaucium, Glycine, Gossypium, Helianthus, Heterocallis, Hevea, Hordeum, Hyoscyamus, Lactuca, Landolphia, Linum, Litsea, Lolium, Lupinus, Lycopersicon, Malus, Manihot, Majorana, Medicago, Musa, Nicotiana, Olea, Oryza, Panicum, Pannesetum, Papaver, Parthenium, Persea, Phaseolus, Pinus, Pistachia, Pisum, Pyrus, Prunus, Raphanus, Rhizocarya, Ricinus, Secale, Senecio, Sinomenium, Sinapis, Solanum, Sorghum, Stephania, Theobroma, Trigonelia, Triticum, Vicia, Vinca, Vitis, Vigna, and Zea.

Particularly suitable plants with which to practice the invention include plants that are capable of producing one or more alkaloids. A “plant that is capable of producing one or more alkaloids” refers to a plant that is capable of producing one or more alkaloids even when it is not transgenic for a regulatory protein described herein. For example, a plant from the Solanaceae or Papaveraceae family is capable of producing one or more alkaloids when it is not transgenic for a regulatory protein described herein. In certain cases, a plant or plant cell may be transgenic for sequences other than the regulatory protein sequences described herein, e.g., growth factors or stress modulators, and can still be characterized as “capable of producing one or more alkaloids,” e.g., a Solanaceae family member transgenic for a growth factor but not transgenic for a regulatory protein described herein.

Useful plant families that are capable of producing one or more alkaloids include the Papaveraceae, Berberidaceae, Lauraceae, Menispermaceae, Euphorbiaceae, Leguminosae, Boraginaceae, Apocynaceae, Asclepiadaceae, Liliaceae, Gnetaceae, Erythroxylaceae, Convolvulaceae, Ranunculaeceae, Rubiaceae, Solanaceae, and Rutaceae families. The Papaveraceae family, for example, contains about 250 species found mainly in the northern temperate regions of the world and includes plants such as California poppy and Opium poppy. Useful genera within the Papaveraceae family include the Papaver (e.g., Papaver bracteatum, Papaver orientate, Papaver setigerum, and Papaver somniferum), Sanguinaria, Dendromecon, Glaucium, Meconopsis, Chelidonium, Eschscholzioideae (e.g., Eschscholzia, Eschscholzia california), and Argemone (e.g., Argemone hispida, Argemone mexicana, and Argemone munita) genera. Other alkaloid producing species with which to practice this invention include Croton salutaris, Croton balsamifera, Sinomenium acutum, Stephania cepharantha, Stephania zippeliana, Litsea sebiferea, Alseodaphne perakensis, Cocculus laurifolius, Duguetia obovata, Rhizocarya racemifera, and Beilschmiedia oreophila, or other species listed in Table 2, below.

### Alkaloid Compounds

Compositions and methods described herein are useful for producing one or more alkaloid compounds. Alkaloid compounds are nitrogenous organic molecules that are typically derived from plants. Alkaloid biosynthetic pathways often include amino acids as reactants. Alkaloid compounds can be mono-, bi-, or polycyclic compounds. Bi- or poly-cyclic compounds can include bridged structures or fused rings. In certain cases, an alkaloid compound can be a plant secondary metabolite.

The regulatory proteins described previously can modulate transcription of sequences involved in the biosynthesis of alkaloid compounds. Thus, a transgenic plant or cell comprising a recombinant nucleic acid expressing such a regulatory protein can be effective for modulating the amount and/or rate of biosynthesis of one or more of such alkaloids in a plant containing the associated regulatory region, either as a genomic sequence or introduced in a recombinant nucleic acid construct.

An amount of one or more of any individual alkaloid compound can be modulated, e.g., increased or decreased, relative to a control plant or cell not transgenic for the particular regulatory protein using the methods described herein. In certain cases, therefore, more than one alkaloid compound (e.g., two, three, four, five, six, seven, eight, nine, ten or even more alkaloid compounds) can have its amount modulated relative to a control plant or cell that is not transgenic for a regulatory protein described herein.

Alkaloid compounds can be grouped into classes based on chemical and structural features. Alkaloid classes described herein include, without limitation, tetrahydrobenzylisoquinoline alkaloids, morphinan alkaloids, benzophenanthridine alkaloids, monoterpenoid indole alkaloids, bisbenzylisoquinoline alkaloids, pyridine alkaloids, purine alkaloids, tropane alkaloids, quinoline alkaloids, terpenoid alkaloids, betaine alkaloids, steroid alkaloids, acridone alkaloids, and phenethylamine alkaloids. Other classifications may be known to those having ordinary skill in the art. Alkaloid compounds whose amounts are modulated relative to a control plant can be from the same alkaloid class or from different alkaloid classes.

In certain embodiments, a morphinan alkaloid compound that is modulated is salutaridine, salutaridinol, salutaridinol acetate, thebaine, isothebaine, papaverine, narcotine, narceine, hydrastine, oripavine, morphinone, morphine, codeine, codeinone, and neopinone. Other morphinan analog alkaloid compounds of interest include sinomenine, flavinine, oreobeiline, and zipperine.

In other embodiments, a tetrahydrobenzylisoquinoline alkaloid compound that is modulated is 2′-norberbamunine, S-coclaurine, S-norcoclaurine, R—N-methyl-coclaurine, S—N-methylcoclaurine, S-3′-hydroxy-N-methylcoclaurine, aromarine, S-3-hydroxycoclaurine, S-norreticuline, R-norreticuline, S-reticuline, R-reticuline, S-scoulerine, S-cheilanthifoline, S-stylopine, S-cis-N-methyl-stylopine, protopine, 6-hydroxy-protopine, 1,2-dehydro-reticuline, S-tetrahydrocolumbamine, columbamine, palmatine, tetrahydropalmatine, S-canadine, berberine, noscapine, S-norlaudenosoline, 6-O-methylnorlaudanosoline, and nororientaline.

In some embodiments, a benzophenanthridine alkaloid compound can be modulated, which can be dihydrosanguinarine, sanguinarine, dihydroxy-dihydro-sanguinarine, 12-hydroxy-dihydrochelirubine, 10-hydroxy-dihydro-sanguinarine, dihydro-macarpine, dihydro-chelirubine, dihydro-sanguinarine, chelirubine, 12-hydroxy-chelirubine, or macarpine.

In yet other embodiments, monoterpenoid indole alkaloid compounds that are modulated include vinblastine, vincristine, yohimbine, ajmalicine, ajmaline, and vincamine. In other cases, a pyridine alkaloid is modulated. A pyridine alkaloid can be piperine, coniine, trigonelline, arecaidine, guvacine, pilocarpine, cytosine, nicotine, and sparteine. A tropane alkaloid that can be modulated includes atropine, cocaine, tropacocaine, hygrine, ecgonine, (−) hyoscyamine, (−) scopolamine, and pelletierine. A quinoline alkaloid that is modulated can be quinine, strychnine, brucine, veratrine, or cevadine. Acronycine is an example of an acridone alkaloid.

In some cases, a phenylethylamine alkaloid can be modulated, which can be MDMA, methamphetamine, mescaline, and ephedrine. In other cases, a purine alkaloid is modulated, such as the xanthines caffeine, theobromine, theacrine, and theophylline.

Bisbenzylisoquinoline alkaloids that can be modulated in amount include (+)tubocurarine, dehatrine, (+)thalicarpine, aromoline, guatteguamerine, berbamunine, and isotetradine. Yet another alkaloid compound that can be modulated in amount is 3,4-dihydroxyphenylacetaldehyde.

Certain useful alkaloid compounds, with associated plant species that are capable of producing them, are listed in Table 2, below.

TABLE 2
Alkaloid Compound Table

| Alkaloid Name | Plant Source(s) |
|---|---|
| Apomorphine | Papaver somniferum |
| Hemsleyadine | Aconitum hemsleyanum, Hemsleya amabilis |
| Anabasine | Anabasis sphylla |
| Aconitine | Aconitum spp. |
| Anisodamine | Anisodus tanguticus |
| Anisodine | Datura sanguinera |
| Arecoline | Areca catechu |
| Atropine | Atropa belladonna, Datura stomonium |
| Homatropine | Atropa belladonna |
| Berberine | Berberis spp. and Mahonia spp. |
| Caffeine | Camellia sinensis, Theobroma cacao, Coffea |
| | arabica, Cola spp. |
| Camptothecin | Camptotheca acuminata |
| Orothecin | Camptotheca acuminata |
| 9-amino camptothecin | Camptotheca acuminata |
| Topotecan | Camptotheca acuminata |
| Irinotecan | Camptotheca acuminata |
| Castanospermine | Castanosperma australe, Alexa spp. |
| Vinblastine | Catharanthus roseus |
| Vincristine | Catharanthus roseus |
| Vinorelbine | Catharanthus roseus |
| Emetine | Alangium lamarkii, Cephaelis ipecacuanha, |
| | Psychotria spp. |
| Homoharringtonine | Cephalotaxus spp. |
| Harringtonine | Cephalotaxus spp. |
| Tubocurarine | Chondodendron tomentosum |
| Quinine | Cinchona officinalis, Cinchona spp., Remijia |
| | pedunculata |
| Quinidine | Cinchona spp., Remijia pedunculata |
| Cissampareine | Cissampelos pareira |
| Cabergoline | Claviceps pupurea |
| Colchicine | Colchicum autumnale |
| Demecolcine | Colchicum spp., Merendera spp. |
| Palmatine | Coptis japonica, Berberis spp., Mahonia spp. |
| Tetrahydropalmatine | Coptis japonica, Berberis spp., Mahonia spp. |
| Monocrotaline | Crotalaria spp. |
| Sparteine | Cytisus scoparius, Sophora pschycarpa, |
| | Ammodendron spp. |
| Changrolin | Dichroa febrifuga |
| Ephedrine | Ephedra sinica, Ephedra spp. |
| Cocaine | Erythroxylum coca |
| Rotundine | Eschsholtzia californica, Stephania sinica, |
| | Eschsholtzia spp., Argemone spp. |
| Galanthamine | Galanthus wornorii |
| Gelsemin | Gelsemium sempervivens |
| Glaucine | Glaucium flavum, Berberis spp. and |
| | Mahonia spp. |
| Indicine | Heliotropium indicum & Messerschmidia |
| | argentea |
| Hydrastine | Hydrastis canadensis |
| Hyoscyamine | Hyoscyamus, Atropa, Datura, Scopolia spp. |
| a-Lobeline | Lobelia spp. |
| Huperzine A | Lycopodium serratum (=Huperzia serrata), |
| | Lycopodium spp. |
| Ecteinascidin 743 | Marine tunicate-Ecteinascidia turbinata |
| Nicotine | Nicotiana tabacum |
| Ellipticine | Ochrosia spp., Aspidospera subincanum, |
| | Bleekeria vitiensis |
| 9-Methoxyellipticine | Ochrosia spp., Excavatia coccinea, Bleekeria |
| | vitiensis |
| Codeine | Papaver somniferum |
| Hydrocodone | Papaver somniferum |
| Hydromorphone | Papaver somniferum |
| Morphine | Papaver somniferum |
| Narceine | Papaver somniferum |
| Oxycodone | Papaver somniferum |
| Oxymorphone | Papaver somniferum |
| Papaverine | Papaver somniferum, Rauwolfia serpentina |
| Thebaine | Papaver bracteatum, Papaver spp. |
| Yohimbine | Pausinystalia yohimbe, Rauwolfia, Vinca, & |
| | Catharanthus spp. |
| Physostigmine | Physostigma venenosum |
| Pilocarpine | Pilocarpus microphyllus, Philocarpus spp. |
| Oxandrin | Pseudoxandra lucida |
| Sarpagine | Rauwolfia & Vinca spp. |
| Deserpidine | Rauwolfia canescens, Rauwolfia spp. |
| Rescinnamine | Rauwolfia spp. |
| Reserpine | Rauwolfia serpentina, Rauwolfia spp. |
| Ajmaline | Rauwolfia serpentina, Rauwolfia spp., Melodinus |
| | balansae, Tonduzia longifolia |
| Ajmalicine | Rauwolfia spp., Vinca rosea |
| Sanguinarine | Sanguinaria canadensis, |
| | Eschscholtzia californica |
| Matrine | Sophora spp. |
| Tetrandrine | Stephania tetrandra |
| Strychnine | Strychnos nux-vomica, Strychnos spp. |
| Brucine | Strychnos spp. |
| Protoveratrines A, B | Veratrum spp. |
| Cyclopamine | Vertatrum spp. |
| Veratramine | Veratrum spp. |
| Vasicine | Vinca minor, Galega officinalis |
| Vindesine | Vinca rosea |
| Vincamine | Vinca spp. |
| Buprenorphine | Papaver somniferum |
| Cimetropium Bromide | Atropa, Datura, Scopolia, Hyoscyamus spp. |
| Levallorphan | Papaver somniferum |
| Serpentine | Rauwolfvia spp. and Catharanthus spp. |
| Noscapine | Papaver somniferum |
| Scopolamine | Atropa, Datura, Scopolia, Hyoscyamus spp. |
| Salutaridine | Croton salutaris, Croton balsamifera, Papaver |
| | spp. and Glaucium spp. |
| Sinomenine | Sinomenium acutum and Stephania cepharantha |
| Flavinine | Litsea sebiferea, Alseodaphne perakensis, |
| | Cocculus laurifolius, Duguetia obovata and |
| | Rhizocarya racemifera |
| Oreobeiline | Beilschmiedia oreophila |
| Zippeline | Stephania zippeliana |

The amount of one or more alkaloid compounds can be increased or decreased in transgenic cells or tissues expressing a regulatory protein as described herein. An increase can be from about 1.5-fold to about 300-fold, or about 2-fold to about 22-fold, or about 50-fold to about 200-fold, or about 75-fold to about 130-fold, or about 5-fold to about 50-fold, or about 5-fold to about 10-fold, or about 10-fold to about 20-fold, or about 150-fold to about 200-fold, or about 20-fold to about 75-fold, or about 10-fold to about 100-fold, or about 40-fold to about 150-fold, about 100-fold to about 200-fold, about 150-fold to about 300-fold, or about 30-fold to about 50-fold higher than the amount in corresponding control cells or tissues that lack the recombinant nucleic acid encoding the regulatory protein.

In other embodiments, the alkaloid compound that is increased in transgenic cells or tissues expressing a regulatory protein as described herein is either not produced or is not detectable in corresponding control cells or tissues that lack the recombinant nucleic acid encoding the regulatory protein. Thus, in such embodiments, the increase in such an alkaloid compound is infinitely high as compared to corresponding control cells or tissues that lack the recombinant nucleic acid encoding the regulatory protein. For example, in certain cases, a regulatory protein described herein may activate a biosynthetic pathway in a plant that is not normally activated or operational in a control plant, and one or more new alkaloids that were not previously produced in that plant species can be produced.

The increase in amount of one or more alkaloids can be restricted in some embodiments to particular tissues and/or organs, relative to other tissues and/or organs. For example, a transgenic plant can have an increased amount of an alkaloid in leaf tissue relative to root or floral tissue.

In other embodiments, the amounts of one or more alkaloids are decreased in transgenic cells or tissues expressing a regulatory protein as described herein. A decrease ratio can be expressed as the ratio of the alkaloid in such a transgenic cell or tissue on a weight basis (e.g., fresh or freeze dried weight basis) as compared to the alkaloid in a corresponding control cell or tissue that lacks the recombinant nucleic acid encoding the regulatory protein. The decrease ratio can be from about 0.05 to about 0.90. In certain cases, the ratio can be from about 0.2 to about 0.6, or from about 0.4 to about 0.6, or from about 0.3 to about 0.5, or from about 0.2 to about 0.4.

In certain embodiments, the alkaloid compound that is decreased in transgenic cells or tissues expressing a regulatory protein as described herein is decreased to an undetectable level as compared to the level in corresponding control cells or tissues that lack the recombinant nucleic acid encoding the regulatory protein. Thus, in such embodiments, the decrease ratio in such an alkaloid compound is zero.

The decrease in amount of one or more alkaloids can be restricted in some embodiments to particular tissues and/or organs, relative to other tissues and/or organs. For example, a transgenic plant can have a decreased amount of an alkaloid in leaf tissue relative to root or floral tissue.

In some embodiments, the amounts of two or more alkaloids are increased and/or decreased, e.g., the amounts of two, three, four, five, six, seven, eight, nine, ten (or more) alkaloid compounds are independently increased and/or decreased. The amount of an alkaloid compound can be determined by known techniques, e.g., by extraction of alkaloid compounds followed by gas chromatography-mass spectrometry (GC-MS) or liquid chromatography-mass spectrometry (LC-MS). If desired, the structure of the alkaloid compound can be confirmed by GC-MS, LC-MS, nuclear magnetic resonance and/or other known techniques.

### Methods of Screening for Associations and Modulating Expression of Sequences of Interest

Provided herein are methods of screening for novel regulatory region-regulatory protein association pairs. The described methods can thus determine whether or not a given regulatory protein can activate a given regulatory region (e.g., to modulate expression of a sequence of interest operably linked to the given regulatory region).

A method of determining whether or not a regulatory region is activated by a regulatory protein can include determining whether or not reporter activity is detected in a plant cell transformed with a recombinant nucleic acid construct comprising a test regulatory region operably linked to a nucleic acid encoding a polypeptide having the reporter activity and with a recombinant nucleic acid construct comprising a nucleic acid encoding a regulatory protein described herein. Detection of the reporter activity indicates that the test regulatory region is activated by the regulatory protein. In certain cases, the regulatory region is a regulatory region as described herein, e.g., comprising a nucleic acid sequence having 80% or greater sequence identity to a regulatory region as set forth in SEQ ID NOs:1453-1468.

For example, a plant can be made that is stably transformed with a sequence encoding a reporter operably linked to the regulatory region under investigation. The plant is inoculated with Agrobacterium containing a sequence encoding a regulatory protein on a Ti plasmid vector. A few days after inoculation, the plant tissue is examined for expression of the reporter, or for detection of reporter activity associated with the reporter. If reporter expression or activity is observed, it can be concluded that the regulatory protein increases transcription of the reporter coding sequence, such as by binding the regulatory region. A positive result indicates that expression of the regulatory protein being tested in a plant would be effective for increasing the in planta amount and/or rate of biosynthesis of one or more sequences of interest operably linked to the associated regulatory region.

Similarly, a method of determining whether or not a regulatory region is activated by a regulatory protein can include determining whether or not reporter activity is detected in a plant cell transformed with a recombinant nucleic acid construct comprising a regulatory region as described herein operably linked to a reporter nucleic acid, and with a recombinant nucleic acid construct comprising a nucleic acid encoding a test regulatory protein. Detection of reporter activity indicates that the regulatory region is activated by the test regulatory protein. In certain cases, the regulatory protein is a regulatory protein as described herein, e.g., comprising a polypeptide sequence having 80% or greater sequence identity to a polypeptide sequence set forth in any of SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NOs:200-203, SEQ ID NOs:205-209, SEQ ID NOs:211-214, SEQ ID NOs:216-223, SEQ ID NOs:225-227, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NOs:632-635, SEQ ID NO:637, SEQ ID NOs:639-646, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NOs:703-709, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-919, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NOs:1212-1218, SEQ ID NOs:1220-1224, SEQ ID NOs:1226-1241, SEQ ID NOs:1243-1246, SEQ ID NOs:1248-1253, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NOs:1394-1399, SEQ ID NOs:1401-1402, SEQ ID NOs:1404-1411, SEQ ID NOs:1413-1419, SEQ ID NO:1421, SEQ ID NOs:1423-1427, SEQ ID NOs:1429-1438, SEQ ID NOs:1440-1450, SEQ ID NO:1452, SEQ ID NOs:1476-1484, or a consensus sequence set forth in any of FIGS. 1-140.

A transformation can be a transient transformation or a stable transformation, as discussed previously. The regulatory region and the nucleic acid encoding a test regulatory protein can be on the same or different nucleic acid constructs.

A reporter activity, such as an enzymatic or optical activity, can permit the detection of the presence of the reporter polypeptide in situ or in vivo, either directly or indirectly. For example, a reporter polypeptide can itself be bioluminescent upon exposure to light. As an alternative, a reporter polypeptide can catalyze a chemical reaction in vivo that yields a detectable product that is localized inside or that is associated with a cell that expresses the chimeric polypeptide. Exemplary bioluminescent reporter polypeptides that emit light in the presence of additional polypeptides, substrates or cofactors include firefly luciferase and bacterial luciferase. Bioluminescent reporter polypeptides that fluoresce in the absence of additional proteins, substrates or cofactors when exposed to light having a wavelength in the range of 300 nm to 600 nm include, for example: amFP486, Mut15-amFP486, Mut32-amFP486, CNFP-MODCd1 and CNFP-MODCd2; asFP600, mut1-RNFP, NE-RNFP, d1RNFP and d2RNFP; cFP484, Δ19-cFP484 and Δ38-cFP484; dgFP512; dmFP592; drFP583, E5 drFP583, E8 drFP583, E5UP drFP583, E5down drFP583, E57 drFP583, AG4 drFP583 and AG4H drFP583; drFP583/dmFP592, drFP583/dmFP592-2G and drFP583/dmFP592-Q3; dsFP483; zFP506, N65M-zFP506, d1zFP506 and d2zFP506; zFP538, M128V-zFPS38, YNFPM128V-MODCd1 and YNFPM128V-MODCd2; GFP; EGFP, ECFP, EYFP, EBFP, BFP2; d4EGFP, d2EGFP, and d1EGFP; and DsRed and DsRed1. See WO 00/34318; WO 00/34320; WO 00/34319; WO 00/34321; WO 00/34322; WO 00/34323; WO 00/34324; WO 00/34325; WO 00/34326; GenBank Accession No. AAB57606; Clontech User Manual, April 1999, PT2040-1, version PR94845; Li et al., J. Biol. Chem. 1998, 273:34970-5; U.S. Pat. No. 5,777,079; and Clontech User Manual, October 1999, PT34040-1, version PR9×217. Reporter polypeptides that catalyze a chemical reaction that yields a detectable product include, for example, β-galactosidase or β-glucuronidase. Other reporter enzymatic activities for use in the invention include neomycin phosphotransferase activity and phosphinotricin acetyl transferase activity.

In some cases, it is known that a particular transcription factor can activate transcription from a particular alkaloid regulatory region(s), e.g., a regulatory region involved in alkaloid biosynthesis. In these cases, similar methods can also be useful to screen other regulatory regions, such as other regulatory regions involved in alkaloid biosynthesis, to determine whether they are activated by the same transcription factor. Thus, the method can comprise transforming a plant cell with a nucleic acid comprising a test regulatory region operably linked to a nucleic acid encoding a polypeptide having reporter activity. The plant cell can include a recombinant nucleic acid encoding a regulatory protein operably linked to a regulatory region that drives transcription of the regulatory protein in the cell. If reporter activity is detected, it can be concluded that the regulatory protein activates transcription mediated by the test regulatory region.

Provided herein also are methods to modulate expression of sequences of interest. Modulation of expression can be expression itself, an increase in expression, or a decrease in expression. Such a method can involve transforming a plant cell with, or growing a plant cell comprising, at least one recombinant nucleic acid construct. A recombinant nucleic acid construct can include a regulatory region as described above, e.g., comprising a nucleic acid having 80% or greater sequence identity to a regulatory region set forth in SEQ ID NOs:1453-1468, where the regulatory region is operably linked to a nucleic acid encoding a sequence of interest. In some cases, a recombinant nucleic acid construct can further include a nucleic acid encoding a regulatory protein as described above, e.g., comprising a polypeptide sequence having 80% or greater sequence identity to a polypeptide sequence set forth in any of SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NOs:200-203, SEQ ID NOs:205-209, SEQ ID NOs:211-214, SEQ ID NOs:216-223, SEQ ID NOs:225-227, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NOs:632-635, SEQ ID NO:637, SEQ ID NOs:639-646, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NOs:703-709, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-919, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:1119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NOs:1212-1218, SEQ ID NOs:1220-1224, SEQ ID NOs:1226-1241, SEQ ID NOs:1243-1246, SEQ ID NOs:1248-1253, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351 SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NOs:1394-1399, SEQ ID NOs:1401-1402, SEQ ID NOs:1404-1411, SEQ ID NOs:1413-1419, SEQ ID NO:1421, SEQ ID NOs:1423-1427, SEQ ID NOs:1429-1438, SEQ ID NOs:1440-1450, SEQ ID NO:1452, SEQ ID NOs:1476-1484, or a consensus sequence set forth in any of FIGS. 1-140. In other cases, the nucleic acid encoding the described regulatory protein is contained on a second recombinant nucleic acid construct. In either case, the regulatory region and the regulatory protein are associated, e.g., as shown in Table 4 (under Example 5 below) or as described herein (e.g., all orthologs of a regulatory protein are also considered to associate with the regulatory regions shown to associate with a given regulatory protein in Table 4, under Example 5 below). A plant cell is typically grown under conditions effective for the expression of the regulatory protein.

As will be recognized by those having ordinary skill in the art, knowledge of an associated regulatory region-regulatory protein pair can also be used to modulate expression of endogenous sequences of interest that are operably linked to endogenous regulatory regions. In such cases, a method of modulating expression of a sequence of interest includes transforming a plant cell that includes an endogenous regulatory region as described herein, with a recombinant nucleic acid construct comprising a nucleic acid encoding a regulatory protein as described herein, where the regulatory region and the regulatory protein are associated as indicated in Table 4 (under Example 5 below) and as described herein. Accordingly, an orthologous sequence and a polypeptide corresponding to the consensus sequence of a given regulatory protein would also be considered to be associated with the regulatory region shown in Table 4 (under Example 5 below) to be associated with the given regulatory protein. A method for expressing an endogenous sequence of interest can include growing such a plant cell under conditions effective for the expression of the regulatory protein. An endogenous sequence of interest can in certain cases be a nucleic acid encoding a polypeptide involved in alkaloid biosynthesis, such as an alkaloid biosynthesis enzyme or a regulatory protein involved in alkaloid biosynthesis.

In other cases, knowledge of an associated regulatory region-regulatory protein pair can be used to modulate expression of exogenous sequences of interest by endogenous regulatory proteins. Such a method can include transforming a plant cell that includes a nucleic acid encoding a regulatory protein as described herein, with a recombinant nucleic acid construct comprising a regulatory region described herein, where the regulatory region is operably linked to a sequence of interest, and where the regulatory region and the regulatory protein are associated as shown in Table 4 (under Example 5 below) and described herein. A method of expressing a sequence of interest can include growing such a plant cell under conditions effective for the expression of the endogenous regulatory protein.

Also provided are methods for producing one or more alkaloids. Such a method can include growing a plant cell that includes a nucleic acid encoding an exogenous regulatory protein as described herein and an endogenous regulatory region as described herein operably linked to a sequence of interest. The regulatory protein and regulatory region are associated, as described previously. A sequence of interest can encode a polypeptide involved in alkaloid biosynthesis. A plant cell can be from a plant capable of producing one or more alkaloids. The plant cell can be grown under conditions effective for the expression of the regulatory protein. The one or more alkaloids produced can be novel alkaloids, e.g., not normally produced in a wild-type plant cell.

Alternatively, a method for producing one or more alkaloids can include growing a plant cell that includes a nucleic acid encoding an endogenous regulatory protein as described herein and a nucleic acid including an exogenous regulatory region as described herein operably linked to a sequence of interest. A sequence of interest can encode a polypeptide involved in alkaloid biosynthesis. A plant cell can be grown under conditions effective for the expression of the regulatory protein. The one or more alkaloids produced can be novel alkaloids, e.g., not normally produced in a wild-type plant cell.

Provided herein also are methods for modulating (e.g., altering, increasing, or decreasing) the amounts of one or more alkaloids in a plant cell. The method can include growing a plant cell as described above, e.g., a plant cell that includes a nucleic acid encoding an endogenous or exogenous regulatory protein, where the regulatory protein associates with, respectively, an exogenous or endogenous regulatory region operably linked to a sequence of interest. In such cases, a sequence of interest can encode a polypeptide involved in alkaloid biosynthesis. Alternatively, a sequence of interest can result in a transcription product such as an antisense RNA or interfering RNA that affects alkaloid biosynthesis pathways, e.g., by modulating the steady-state level of mRNA transcripts available for translation that encode one or more alkaloid biosynthesis enzymes.

The invention will be further described in the following examples, which do not limit the scope of the invention described in the claims.

### EXAMPLES

### Example 1

### Generation of Arabidopsis Plants Containing Alkaloid Regulatory Region::Luciferase Constructs

T-DNA binary vector constructs were made using standard molecular biology techniques. A set of constructs were made that contained a luciferase coding sequence operably linked to one or two of the regulatory regions set forth in SEQ ID NOs:1453-1457, SEQ ID NOs:1459-1463, SEQ ID NO:1465, and SEQ ID NOs:1467-1468. Each of these constructs also contained a marker gene conferring resistance to the herbicide Finale®.

Each construct was introduced into Arabidopsis ecotype Wassilewskija (WS) by the floral dip method essentially as described in Bechtold et al., C.R. Acad. Sci. Paris, 316:1194-1199 (1993). The presence of each reporter region::luciferase construct was verified by PCR. At least two independent events from each transformation were selected for further study; these events were referred to as Arabidopsis thaliana screening lines. T₁ (first generation transformant) seeds were germinated and allowed to self-pollinate. T₂ (second generation, progeny of self-pollinated T₁ plants) seeds were collected and a portion were germinated and allowed to self-pollinate. T₃ (third generation, progeny of self-pollinated T₂ plants) seeds were collected.

### Example 2

### Screening of Regulatory Proteins in Arabidopsis

T₂ or T₃ seeds of the Arabidopsis thaliana screening lines described in Example 1 were planted in soil comprising Sunshine LP5 Mix and Thermorock Vermiculite Medium #3 at a ratio of 60:40, respectively. The seeds were stratified at 4° C. for approximately two to three days. After stratification, the seeds were transferred to the greenhouse and covered with a plastic dome and tarp until most of the seeds had germinated. Plants were grown under long day conditions. Approximately seven to ten days post-germination, plants were sprayed with Finales herbicide to confirm that the plants were transgenic. Between three to four weeks after germination, the plants were used for screening.

T-DNA binary vector constructs comprising a CaMV 35S constitutive promoter operably linked to one of the regulatory protein coding sequences listed in Table 4 (under Example 5 below) were made and transformed into Agrobacterium. One colony from each transformation was selected and maintained as a glycerol stock. Two days before the experiment commenced, each transformant was inoculated into 150 μL of YEB broth containing 100 μg/mL spectinomycin, 50 μg/mL rifampicin, and 20 μM acetosyringone; grown in an incubator-shaker at 28° C.; and harvested by centrifugation at 4,000 rpm for at least 25 minutes. The supernatant was discarded, and each pellet was resuspended in a solution of 10 mM MgCl; 10 mM MES, pH 5.7; and 150 μM acetosyringone to an optical density (OD₆₀₀) of approximately 0.05 to 0.1. Each suspension was transferred to a 1 mL syringe outfitted with a 30 gauge needle.

Plants were infected by mildly wounding the surface of a leaf using the tip of a syringe/needle containing a suspension of one of the Agrobacterium transformants. A small droplet of the Agrobacterium suspension was placed on the wound area after wounding. Each leaf was wounded approximately 10 times at different positions on the same leaf. Each leaf was wounded using one Agrobacterium transformant. The syringe needle preferably did not pierce through the leaf to increase the likelihood of Agrobacterium infection on the wounded site. Treated leaves were left attached to the mother plant for at least 5 days prior to analysis.

### Example 3

### Screening of Regulatory Proteins in Nicotiana

Stable Nicotiana tabacum, cultivar Samsun, screening lines were generated by transforming Nicoliana leaf explants separately with the T-DNA binary vector constructs containing a luciferase reporter gene operably linked to one or two regulatory regions described in Example 1, following the transformation protocol essentially as described by Rogers et al., Methods in Enzymology 118:627 (1987). Leaf disks were cut from leaves of the screening lines using a paper puncher and were transiently infected with Agrobacterium clones prepared as described in Example 2. In addition, leaf disks from wild-type Nicotiana tabacum plants, cultivar SR1, were transiently infected with Agrobacterium containing a binary vector comprising a CaMV 35S constitutive promoter operably linked to a luciferase reporter coding sequence. These leaf disks were used as positive controls to indicate that the method of Agrobacterium infection was working. Some leaf disks from Nicotiana screening plants were transiently infected with Agrobacterium containing a binary construct of a CaMV 35S constitutive promoter operably linked to a GFP coding sequence. These leaf disks served as reference controls to indicate that the luciferase reporter activity in the treated disks was not merely a response to treatment with Agrobacterium.

Transient infection was performed by immersing the leaf disks in about 5 to 10 mL of a suspension of Agrobacterium culture, prepared as described in Example 2, for about 2 min. Treated leaf disks were briefly and quickly blot-dried in tissue paper and then transferred to a plate lined with paper towels sufficiently wet with 1×MS solution (adjusted to pH 5.7 with 1 N KOH and supplemented with 1 mg/L BAP and 0.25 mg/L NAA). The leaf disks were incubated in a growth chamber under long-day light/dark cycle at 22° C. for 5 days prior to analysis.

### Example 4

### Co-Infection Experiments in Nicotiana

In some cases, a mixture of two different Agrobacterium cultures was used in transient co-infection experiments in wild-type Nicotiana plants. One of the Agrobacterium cultures contained a vector comprising a regulatory region of interest operably linked to a luciferase reporter gene, and the other contained a vector that included the CaMV 35S constitutive promoter operably linked to a nucleotide sequence that coded for a regulatory factor of interest. The Agrobacterium culture and suspension were prepared as described in Example 2. The two different Agrobacterium suspensions were mixed to a final optical density (OD₆₀₀) of approximately 0.1 to 0.5. The mixture was loaded into a 1 mL syringe with a 30 gauge needle.

Depending on the size of a Nicotiana leaf, it can be divided arbitrarily into several sectors, with each sector accommodating one type of Agrobacterium mixture. Transient infection of a wild-type tobacco leaf sector was done by mildly wounding the surface of a leaf using the tip of a syringe/needle containing a mixture of Agrobacterium culture suspensions. A small droplet of the Agrobacterium suspension was placed on the wound area after wounding. Each leaf sector was wounded approximately 20 times at different positions within the same leaf sector. Treated Nicotiana leaves were left intact and attached to the mother plant for at least 5 days prior to analysis. A leaf sector treated with Agrobacterium that contained a binary construct including a CaMV 35S constitutive promoter operably linked to a GFP coding sequence was used as a reference control.

### Example 5

### Luciferase Assay and Results

Treated intact leaves from Examples 2 and 4, and leaf disks from Example 3, were collected five days after infection and placed in a square Petri dish. Each leaf was sprayed with 10 μM luciferin in 0.01% Triton X-100. Leaves were then incubated in the dark for at least a minute prior to imaging with a Night Owl™ CCD camera from Berthold Technology. The exposure time depended on the screening line being tested; in most cases the exposure time was between 2 to 5 minutes. Qualitative scoring of luciferase reporter activity from each infected leaf was done by visual inspection and comparison of images, taking into account the following criteria: (1) if the luminescence signal was higher in the treated leaf than in the 35S-GFP-treated reference control (considered the background activity of the regulatory region), and (2) if the #1 criterion occurred in at least two independent transformation events carrying the regulatory region-luciferase reporter construct. Results of the visual inspection were noted according to the rating system given in Table 3, and with respect to both the positive and negative controls.

TABLE 3
Luciferase activity scoring system

| Score | Score Comment |
|---|---|
| ++ | signal in the treated leaf is much stronger than in reference |
| | background |
| + | signal in the treated leaf is stronger than in reference background |
| +/− | weak signal but still relatively higher than reference background |
| − | no response |

Alkaloid regulatory region/regulatory protein combinations that resulted in a score of +/−, + or ++ in both independent Arabidopsis transformation events were scored as having detectable luciferase reporter activity. Combinations that resulted in a score of +/−, + or ++ in one independent Arabidopsis transformation event were also scored as having detectable reporter activity if similar ratings were observed in the Nicotiana experiment. Combinations (also referred to as associations herein) having detectable luciferase reporter activity are shown in Table 4, below.

TABLE 4
Combinations of regulatory regions and regulatory proteins producing
expression of a reporter gene operably linked to each regulatory region

| | | | | |
|---|---|---|---|---|
| Regulatory | Regulatory | Regulatory | Regulatory | |
| Region | Protein | Protein | Protein | |
| Construct | SEQ ID NO: | Gemini_ID | cDNA_ID | Screening Organism |

| | | | | |
|---|---|---|---|---|
| AtBBE2-L- | 80 | 538B12 | 23798983 | Arabidopsis thaliana |
| AtBBE5-K | | | | |
| AtBBE2-L- | 1440 | 539D12 | 23814706 | Arabidopsis thaliana |
| AtBBE5-K | | | | |
| AtBBE2-L- | 86 | 539D8 | 23389356 | Arabidopsis thaliana |
| AtBBE5-K | | | | |
| AtBBE2-L- | 93 | 540C1 | 23819377 | Arabidopsis thaliana |
| AtBBE5-K | | | | |
| AtBBE2-L- | 95 | 540E8 | 23693590 | Arabidopsis thaliana |
| AtBBE5-K | | | | |
| AtBBE2-L- | 113 | 540G4 | 23698626 | Arabidopsis thaliana |
| AtBBE5-K | | | | |
| AtBBE2-L- | 115 | 540G9 | 23663607 | Arabidopsis thaliana |
| AtBBE5-K | | | | |
| AtCR2-L- | 121 | 5109A6 | 23548978 | Arabidopsis thaliana |
| AtROX6-L | | | | and Tobacco |
| AtCR2-L- | 123 | 5109D12 | 23522096 | Arabidopsis thaliana |
| AtROX6-L | | | | |
| AtCR2-L- | 141 | 5109E7 | 23447462 | Arabidopsis thaliana |
| AtROX6-L | | | | and Tobacco |
| AtCR2-L- | 144 | 5109F10 | 23499985 | Arabidopsis thaliana |
| AtROX6-L | | | | and Tobacco |
| AtCR2-L- | 152 | 5109F11 | 23651179 | Arabidopsis thaliana |
| AtROX6-L | | | | and Tobacco |
| AtCR2-L- | 158 | 5109G4 | 24374230 | Arabidopsis thaliana |
| AtROX6-L | | | | |
| AtCR2-L- | 168 | 5109G9 | 23547976 | Arabidopsis thaliana |
| AtROX6-L | | | | and Tobacco |
| AtCR2-L- | 1401 | 5109H5 | 23368864 | Arabidopsis thaliana |
| AtROX6-L | | | | |
| AtCR2-L- | 173 | 5110A5 | 13653045 | Arabidopsis thaliana |
| AtROX6-L | | | | |
| AtCR2-L- | 187 | 5110B9 | 23477523 | Tobacco |
| AtROX6-L | | | | |
| AtCR2-L- | 200 | 5110E11 | 13610509 | Arabidopsis thaliana |
| AtROX6-L | | | | and Tobacco |
| AtCR2-L- | 205 | 5110F5 | 23503364 | Arabidopsis thaliana |
| AtROX6-L | | | | and Tobacco |
| AtCR2-L- | 211 | 5110F8 | 12676498 | Tobacco |
| AtROX6-L | | | | |
| AtCR2-L- | 216 | 5110G8 | 4984839 | Tobacco |
| AtROX6-L | | | | |
| AtCR2-L- | 225 | 5111D1 | 23544026 | Arabidopsis thaliana |
| AtROX6-L | | | | and Tobacco |
| AtCR2-L- | 229 | 5111E1 | 13579142 | Arabidopsis thaliana |
| AtROX6-L | | | | and Tobacco |
| AtCR2-L- | 235 | 531A10 | 23365150 | Arabidopsis thaliana |
| AtROX6-L | | | | |
| AtCR2-L- | 246 | 531C11 | 23411827 | Arabidopsis thaliana |
| AtROX6-L | | | | |
| AtCR2-L- | 260 | 531G6 | 23370190 | Arabidopsis thaliana |
| AtROX6-L | | | | |
| AtCR2-L- | 264 | 532A11 | 23367111 | Arabidopsis thaliana |
| AtROX6-L | | | | |
| AtCR2-L- | 284 | 532C11 | 23364997 | Arabidopsis thaliana |
| AtROX6-L | | | | |
| AtCR2-L- | 288 | 534B11 | 23376150 | Arabidopsis thaliana |
| AtROX6-L | | | | |
| AtCR2-L- | 301 | 534C3 | 23649144 | Arabidopsis thaliana |
| AtROX6-L | | | | |
| AtCR2-L- | 309 | 534H2 | 23370269 | Arabidopsis thaliana |
| AtROX6-L | | | | |
| AtCR2-L- | 325 | 537C8 | 23420310 | Arabidopsis thaliana |
| AtROX6-L | | | | |
| AtCR2-L- | 333 | 537F10 | 23764087 | Arabidopsis thaliana |
| AtROX6-L | | | | |
| AtCR2-L- | 345 | 537F8 | 23460392 | Arabidopsis thaliana |
| AtROX6-L | | | | |
| AtCR2-L- | 350 | 537G6 | 23419606 | Arabidopsis thaliana |
| AtROX6-L | | | | |
| AtCR2-L- | 356 | 538B5 | 23740209 | Arabidopsis thaliana |
| AtROX6-L | | | | |
| AtCR2-L- | 364 | 539A8 | 23374089 | Arabidopsis thaliana |
| AtROX6-L | | | | |
| AtCR2-L- | 368 | 540C4 | 23692994 | Arabidopsis thaliana |
| AtROX6-L | | | | and Tobacco |
| AtCR2-L- | 370 | 540C6 | 23666854 | Arabidopsis thaliana |
| AtROX6-L | | | | |
| AtCR2-L- | 376 | 540E4 | 23662829 | Arabidopsis thaliana |
| AtROX6-L | | | | |
| AtCR2-L- | 382 | 540H6 | 23698996 | Arabidopsis thaliana |
| AtROX6-L | | | | |
| AtCR2-L- | 387 | 554A7 | 23369491 | Arabidopsis thaliana |
| AtROX6-L | | | | and Tobacco |
| AtCR2-L- | 392 | 554B7 | 23384563 | Arabidopsis thaliana |
| AtROX6-L | | | | and Tobacco |
| AtCR2-L- | 401 | 554C7 | 23389848 | Arabidopsis thaliana |
| AtROX6-L | | | | and Tobacco |
| AtCR2-L- | 411 | 554D7 | 23384591 | Arabidopsis thaliana |
| AtROX6-L | | | | and Tobacco |
| AtCR2-L- | 419 | 554F8 | 23382112 | Arabidopsis thaliana |
| AtROX6-L | | | | and Tobacco |
| AtCR2-L- | 434 | 554H10 | 23389418 | Arabidopsis thaliana |
| AtROX6-L | | | | and Tobacco |
| AtCR2-L- | 450 | 555A10 | 23374668 | Arabidopsis thaliana |
| AtROX6-L | | | | and Tobacco |
| AtCR2-L- | 458 | 555B2 | 23365920 | Arabidopsis thaliana |
| AtROX6-L | | | | |
| AtCR2-L- | 466 | 555D2 | 23370421 | Arabidopsis thaliana |
| AtROX6-L | | | | |
| AtCR2-L- | 472 | 555D3 | 23783423 | Arabidopsis thaliana |
| AtROX6-L | | | | and Tobacco |
| AtCR2-L- | 1413 | 555F1 | 23374628 | Arabidopsis thaliana |
| AtROX6-L | | | | |
| AtCR2-L- | 490 | 555F5 | 23357171 | Arabidopsis thaliana |
| AtROX6-L | | | | and Tobacco |
| AtROX7-L | 492 | 5109A2 | 23500965 | Arabidopsis thaliana |
| AtROX7-L | 494 | 5109B2 | 23538950 | Arabidopsis thaliana |
| | | | | and Tobacco |
| AtROX7-L | 506 | 5109E11 | 24373996 | Arabidopsis thaliana |
| | | | | and Tobacco |
| AtSS1-L- | 516 | 5110C6 | 23539673 | Arabidopsis thaliana |
| AtWDC-K | | | | |
| AtSS1-L- | 211 | 5110F8 | 12676498 | Arabidopsis thaliana |
| AtWDC-K | | | | |
| AtSS1-L- | 523 | 536H6 | 23357846 | Arabidopsis thaliana |
| AtWDC-K | | | | |
| AtSS1-L- | 532 | 553G11 | 12680548 | Tobacco |
| AtWDC-K | | | | |
| AtSS1-L- | 548 | 555C9 | 23357564 | Arabidopsis thaliana |
| AtWDC-K | | | | and Tobacco |
| AtSS3-L- | 1423 | 5109A1 | 23516818 | Arabidopsis thaliana |
| AtROX7-L | | | | |
| AtSS3-L- | 563 | 5109A11 | 23502516 | Arabidopsis thaliana |
| AtROX7-L | | | | and Tobacco |
| AtSS3-L- | 565 | 5109A5 | 23660778 | Arabidopsis thaliana |
| AtROX7-L | | | | |
| AtSS3-L- | 570 | 5109B1 | 23493156 | Arabidopsis thaliana |
| AtROX7-L | | | | |
| AtSS3-L- | 572 | 5109C1 | 23518770 | Arabidopsis thaliana |
| AtROX7-L | | | | and Tobacco |
| AtSS3-L- | 574 | 5109C6 | 23653450 | Arabidopsis thaliana |
| AtROX7-L | | | | and Tobacco |
| AtSS3-L- | 579 | 5109D1 | 23467847 | Arabidopsis thaliana |
| AtROX7-L | | | | |
| AtSS3-L- | 590 | 5109E12 | 23519948 | Arabidopsis thaliana |
| AtROX7-L | | | | and Tobacco |
| AtSS3-L- | 593 | 5109E2 | 23553534 | Arabidopsis thaliana |
| AtROX7-L | | | | and Tobacco |
| AtSS3-L- | 599 | 5109F2 | 23498294 | Arabidopsis thaliana |
| AtROX7-L | | | | and Tobacco |
| AtSS3-L- | 608 | 5109H10 | 23529931 | Arabidopsis thaliana |
| AtROX7-L | | | | and Tobacco |
| AtSS3-L- | 613 | 5109H3 | 23498685 | Arabidopsis thaliana |
| AtROX7-L | | | | |
| AtSS3-L- | 619 | 5110A1 | 23515088 | Arabidopsis thaliana |
| AtROX7-L | | | | |
| AtSS3-L- | 632 | 5110A2 | 24375036 | Arabidopsis thaliana |
| AtROX7-L | | | | and Tobacco |
| AtSS3-L- | 637 | 5110A8 | 23503138 | Arabidopsis thaliana |
| AtROX7-L | | | | |
| AtSS3-L- | 639 | 5110B1 | 23544992 | Arabidopsis thaliana |
| AtROX7-L | | | | and Tobacco |
| AtSS3-L- | 648 | 5110B2 | 23517564 | Arabidopsis thaliana |
| AtROX7-L | | | | |
| AtSS3-L- | 652 | 5110B7 | 23502669 | Arabidopsis thaliana |
| AtROX7-L | | | | |
| AtSS3-L- | 657 | 5110C1 | 23528916 | Arabidopsis thaliana |
| AtROX7-L | | | | |
| AtSS3-L- | 659 | 5110D5 | 23515246 | Arabidopsis thaliana |
| AtROX7-L | | | | and Tobacco |
| AtSS3-L- | 664 | 5110E4 | 24380616 | Arabidopsis thaliana |
| AtROX7-L | | | | |
| AtSS3-L- | 671 | 5110E6 | 23503971 | Arabidopsis thaliana |
| AtROX7-L | | | | |
| AtSS3-L- | 674 | 5110E7 | 23467433 | Arabidopsis thaliana |
| AtROX7-L | | | | |
| AtSS3-L- | 1380 | 5110F1 | 1823190 | Arabidopsis thaliana |
| AtROX7-L | | | | |
| AtSS3-L- | 679 | 5110F3 | 23554709 | Arabidopsis thaliana |
| AtROX7-L | | | | |
| AtSS3-L- | 686 | 5110F4 | 23524514 | Arabidopsis thaliana |
| AtROX7-L | | | | |
| AtSS3-L- | 205 | 5110F5 | 23503364 | Arabidopsis thaliana |
| AtROX7-L | | | | |
| AtSS3-L- | 695 | 5110G1 | 23503210 | Arabidopsis thaliana |
| AtROX7-L | | | | |
| AtSS3-L- | 698 | 5110G5 | 23494809 | Arabidopsis thaliana |
| AtROX7-L | | | | |
| AtSS3-L- | 216 | 5110G8 | 4984839 | Arabidopsis thaliana |
| AtROX7-L | | | | and Tobacco |
| AtSS3-L- | 701 | 5111A1 | 23512013 | Arabidopsis thaliana |
| AtROX7-L | | | | |
| AtSS3-L- | 703 | 552A10 | 23740916 | Arabidopsis thaliana |
| AtROX7-L | | | | and Tobacco |
| AtSS3-L- | 711 | 552A12 | 23363175 | Arabidopsis thaliana |
| AtROX7-L | | | | |
| AtSS3-L- | 716 | 552A2 | 23421865 | Arabidopsis thaliana |
| AtROX7-L | | | | and Tobacco |
| AtSS3-L- | 721 | 552A7 | 23417641 | Arabidopsis thaliana |
| AtROX7-L | | | | and Tobacco |
| AtSS3-L- | 732 | 552B10 | 23751471 | Arabidopsis thaliana |
| AtROX7-L | | | | and Tobacco |
| AtSS3-L- | 748 | 552B6 | 23773450 | Arabidopsis thaliana |
| AtROX7-L | | | | and Tobacco |
| AtSS3-L- | 760 | 552C6 | 23760303 | Arabidopsis thaliana |
| AtROX7-L | | | | and Tobacco |
| AtSS3-L- | 766 | 552C7 | 23772039 | Arabidopsis thaliana |
| AtROX7-L | | | | and Tobacco |
| AtSS3-L- | 769 | 552D1 | 23792467 | Arabidopsis thaliana |
| AtROX7-L | | | | and Tobacco |
| AtSS3-L- | 777 | 552E6 | 23401404 | Arabidopsis thaliana |
| AtROX7-L | | | | and Tobacco |
| AtSS3-L- | 792 | 552G11 | 23365746 | Arabidopsis thaliana |
| AtROX7-L | | | | and Tobacco |
| AtSS3-L- | 797 | 552G5 | 23765347 | Arabidopsis thaliana |
| AtROX7-L | | | | and Tobacco |
| AtSS3-L- | 812 | 552G7 | 23768927 | Tobacco |
| AtROX7-L | | | | |
| AtSS3-L- | 820 | 552G8 | 23751503 | Arabidopsis thaliana |
| AtROX7-L | | | | |
| EcBBE-L- | 1421 | 5109A7 | 23509990 | Arabidopsis thaliana |
| EcNMCH3-L | | | | |
| EcBBE-L- | 822 | 5109D9 | 23495742 | Arabidopsis thaliana |
| EcNMCH3-L | | | | and Tobacco |
| EcBBE-L- | 828 | 5109E10 | 23523867 | Arabidopsis thaliana |
| EcNMCH3-L | | | | |
| EcBBE-L- | 834 | 5109E3 | 23516633 | Arabidopsis thaliana |
| EcNMCH3-L | | | | |
| EcBBE-L- | 639 | 5110B1 | 23544992 | Arabidopsis thaliana |
| EcNMCH3-L | | | | and Tobacco |
| EcBBE-L- | 840 | 5110B10 | 23505323 | Arabidopsis thaliana |
| EcNMCH3-L | | | | and Tobacco |
| EcBBE-L- | 1452 | 5110C2 | 2706717 | Arabidopsis thaliana |
| EcNMCH3-L | | | | |
| EcBBE-L- | 845 | 5110C3 | 23492765 | Arabidopsis thaliana |
| EcNMCH3-L | | | | |
| EcBBE-L- | 851 | 5110C4 | 23486285 | Arabidopsis thaliana |
| EcNMCH3-L | | | | |
| EcBBE-L- | 856 | 5110D4 | 23499964 | Arabidopsis thaliana |
| EcNMCH3-L | | | | and Tobacco |
| EcBBE-L- | 869 | 5110E1 | 23543586 | Arabidopsis thaliana |
| EcNMCH3-L | | | | and Tobacco |
| EcBBE-L- | 1394 | 5110E2 | 23368554 | Arabidopsis thaliana |
| EcNMCH3-L | | | | |
| EcBBE-L- | 871 | 5110F2 | 4950532 | Arabidopsis thaliana |
| EcNMCH3-L | | | | and Tobacco |
| EcBBE-L- | 205 | 5110F5 | 23503364 | Arabidopsis thaliana |
| EcNMCH3-L | | | | and Tobacco |
| EcBBE-L- | 216 | 5110G8 | 4984839 | Arabidopsis thaliana |
| EcNMCH3-L | | | | |
| EcBBE-L- | 874 | 531A11 | 23397999 | Arabidopsis thaliana |
| EcNMCH3-L | | | | and Tobacco |
| EcBBE-L- | 889 | 531A5 | 23556617 | Arabidopsis thaliana |
| EcNMCH3-L | | | | |
| EcBBE-L- | 906 | 531A9 | 23557650 | Arabidopsis thaliana |
| EcNMCH3-L | | | | and Tobacco |
| EcBBE-L- | 921 | 531B5 | 23385560 | Arabidopsis thaliana |
| EcNMCH3-L | | | | and Tobacco |
| EcBBE-L- | 931 | 531B7 | 23389966 | Arabidopsis thaliana |
| EcNMCH3-L | | | | |
| EcBBE-L- | 946 | 531C10 | 23766279 | Tobacco |
| EcNMCH3-L | | | | |
| EcBBE-L- | 964 | 531D10 | 23746932 | Arabidopsis thaliana |
| EcNMCH3-L | | | | |
| EcBBE-L- | 973 | 531D4 | 23380615 | Arabidopsis thaliana |
| EcNMCH3-L | | | | and Tobacco |
| EcBBE-L- | 983 | 531D8 | 23366147 | Arabidopsis thaliana |
| EcNMCH3-L | | | | and Tobacco |
| EcBBE-L- | 992 | 531E3 | 23416775 | Arabidopsis thaliana |
| EcNMCH3-L | | | | |
| EcBBE-L- | 1001 | 531E8 | 23359888 | Arabidopsis thaliana |
| EcNMCH3-L | | | | and Tobacco |
| EcBBE-L- | 1019 | 531E9 | 23385230 | Arabidopsis thaliana |
| EcNMCH3-L | | | | and Tobacco |
| EcBBE-L- | 1026 | 531F3 | 23359443 | Arabidopsis thaliana |
| EcNMCH3-L | | | | |
| EcBBE-L- | 1042 | 531F7 | 23386664 | Arabidopsis thaliana |
| EcNMCH3-L | | | | and Tobacco |
| EcBBE-L- | 1058 | 531G3 | 23371818 | Arabidopsis thaliana |
| EcNMCH3-L | | | | and Tobacco |
| EcBBE-L- | 260 | 531G6 | 23370190 | Arabidopsis thaliana |
| EcNMCH3-L | | | | |
| EcBBE-L- | 1068 | 531G7 | 23471864 | Arabidopsis thaliana |
| EcNMCH3-L | | | | and Tobacco |
| EcBBE-L- | 1074 | 532A5 | 23370870 | Arabidopsis thaliana |
| EcNMCH3-L | | | | |
| EcBBE-L- | 1087 | 532B5 | 23361688 | Arabidopsis thaliana |
| EcNMCH3-L | | | | and Tobacco |
| EcBBE-L- | 1102 | 533B2 | 23448883 | Arabidopsis thaliana |
| EcNMCH3-L | | | | |
| EcBBE-L- | 1404 | 533C2 | 23372744 | Arabidopsis thaliana |
| EcNMCH3-L | | | | |
| EcBBE-L- | 1119 | 533D2 | 23389186 | Arabidopsis thaliana |
| EcNMCH3-L | | | | |
| EcBBE-L- | 1127 | 533E1 | 23380898 | Arabidopsis thaliana |
| EcNMCH3-L | | | | |
| EcBBE-L- | 1138 | 533F7 | 23383311 | Arabidopsis thaliana |
| EcNMCH3-L | | | | |
| EcBBE-L- | 1147 | 533G4 | 23384792 | Arabidopsis thaliana |
| EcNMCH3-L | | | | |
| EcBBE-L- | 1158 | 533G7 | 23360311 | Arabidopsis thaliana |
| EcNMCH3-L | | | | and Tobacco |
| EcBBE-L- | 1165 | 534C5 | 23375896 | Arabidopsis thaliana |
| EcNMCH3-L | | | | |
| EcBBE-L- | 1171 | 534C6 | 23376628 | Arabidopsis thaliana |
| EcNMCH3-L | | | | |
| EcBBE-L- | 1178 | 534G2 | 23369842 | Arabidopsis thaliana |
| EcNMCH3-L | | | | and Tobacco |
| EcBBE-L- | 1382 | 534H5 | 23367406 | Arabidopsis thaliana |
| EcNMCH3-L | | | | |
| EcBBE-L- | 1192 | 534H6 | 23416869 | Arabidopsis thaliana |
| EcNMCH3-L | | | | |
| EcBBE-L- | 1202 | 536D8 | 23785125 | Arabidopsis thaliana |
| EcNMCH3-L | | | | |
| EcBBE-L- | 1210 | 540E5 | 23694932 | Arabidopsis thaliana |
| EcNMCH3-L | | | | and Tobacco |
| EcBBE-L- | 1212 | 540F5 | 23699071 | Arabidopsis thaliana |
| EcNMCH3-L | | | | and Tobacco |
| EcBBE-L- | 716 | 552A2 | 23421865 | Arabidopsis thaliana |
| EcNMCH3-L | | | | |
| EcBBE-L- | 721 | 552A7 | 23417641 | Arabidopsis thaliana |
| EcNMCH3-L | | | | and Tobacco |
| EcBBE-L- | 1220 | 552B7 | 23527182 | Arabidopsis thaliana |
| EcNMCH3-L | | | | and Tobacco |
| EcBBE-L- | 1226 | 552C1 | 23747378 | Arabidopsis thaliana |
| EcNMCH3-L | | | | and Tobacco |
| EcBBE-L- | 769 | 552D1 | 23792467 | Arabidopsis thaliana |
| EcNMCH3-L | | | | and Tobacco |
| EcBBE-L- | 1429 | 552E4 | 23699979 | Arabidopsis thaliana |
| EcNMCH3-L | | | | and Tobacco |
| EcBBE-L- | 1243 | 552F4 | 23691708 | Arabidopsis thaliana |
| EcNMCH3-L | | | | and Tobacco |
| EcBBE-L- | 1248 | 552G2 | 23697027 | Arabidopsis thaliana |
| EcNMCH3-L | | | | and Tobacco |
| EcBBE-L- | 1255 | 554A10 | 23416843 | Arabidopsis thaliana |
| EcNMCH3-L | | | | |
| EcBBE-L- | 1261 | 555C1 | 23449314 | Arabidopsis thaliana |
| EcNMCH3-L | | | | |
| EcBBE-L- | 1279 | 555C7 | 23390282 | Arabidopsis thaliana |
| EcNMCH3-L | | | | and Tobacco |
| EcBBE-L- | 466 | 555D2 | 23370421 | Arabidopsis thaliana |
| EcNMCH3-L | | | | |
| EcBBE-L- | 1297 | 555E1 | 23380202 | Arabidopsis thaliana |
| EcNMCH3-L | | | | and Tobacco |
| EcBBE-L- | 1310 | 555F2 | 23396143 | Arabidopsis thaliana |
| EcNMCH3-L | | | | |
| PsBBE-L | 572 | 5109C1 | 23518770 | Tobacco |
| PsBBE-L | 563 | 5109A11 | 23502516 | Tobacco |
| PsBBE-L | 229 | 5111E1 | 13579142 | Tobacco |
| PsBBE-L | 983 | 531D8 | 23366147 | Tobacco |
| PsBBE-L | 973 | 531D4 | 23380615 | Tobacco |
| PsBBE-L | 1138 | 533F7 | 23383311 | Tobacco |
| PsBBE-L | 411 | 554D7 | 23384591 | Tobacco |
| PsBBE-L | 1321 | 533G1 | 23389279 | Tobacco |
| PsBBE-L | 350 | 537G6 | 23419606 | Tobacco |
| PsBBE-L | 325 | 537C8 | 23420310 | Tobacco |
| PsBBE-L | 1323 | 552F6 | 23420963 | Tobacco |
| PsBBE-L | 889 | 531A5 | 23556617 | Tobacco |
| PsBBE-L | 370 | 540C6 | 23666854 | Tobacco |
| PsBBE-L | 368 | 540C4 | 23692994 | Tobacco |
| PsBBE-L | 356 | 538B5 | 23740209 | Tobacco |
| PsBBE-L | 703 | 552A10 | 23740916 | Tobacco |
| PsBBE-L | 964 | 531D10 | 23746932 | Tobacco |
| PsBBE-L | 1226 | 552C1 | 23747378 | Tobacco |
| PsBBE-L | 732 | 552B10 | 23751471 | Tobacco |
| PsBBE-L | 80 | 538B12 | 23798983 | Tobacco |
| PsBBE-L | 1335 | 553H9 | 23369680 | Tobacco |
| PsBBE-L | 1340 | 531A4 | 23373703 | Tobacco |
| PsBBE-L | 1342 | 555A2 | 23449316 | Tobacco |
| PsHMCOMT2-L | 828 | 5109E10 | 23523867 | Tobacco |
| PsHMCOMT2-L | 613 | 5109H3 | 23498685 | Tobacco |
| PsHMCOMT2-L | 1158 | 533G7 | 23360311 | Tobacco |
| PsHMCOMT2-L | 281 | 532C11 | 23364997 | Tobacco |
| PsHMCOMT2-L | 264 | 532A11 | 23367111 | Tobacco |
| PsHMCOMT2-L | 1178 | 534G2 | 23369842 | Tobacco |
| PsHMCOMT2-L | 309 | 534H2 | 23370269 | Tobacco |
| PsHMCOMT2-L | 411 | 554D7 | 23384591 | Tobacco |
| PsHMCOMT2-L | 350 | 537G6 | 23419606 | Tobacco |
| PsHMCOMT2-L | 1068 | 531G7 | 23471864 | Tobacco |
| PsHMCOMT2-L | 648 | 5110B2 | 23517564 | Tobacco |
| PsHMCOMT2-L | 376 | 540E4 | 23662829 | Tobacco |
| PsHMCOMT2-L | 370 | 540C6 | 23666854 | Tobacco |
| PsHMCOMT2-L | 703 | 552A10 | 23740916 | Tobacco |
| PsHMCOMT2-L | 1226 | 552C1 | 23747378 | Tobacco |
| PsHMCOMT2-L | 769 | 552D1 | 23792467 | Tobacco |
| PsHMCOMT2-L | 1351 | 553D1 | 23557531 | Tobacco |
| PsROMT-L | 572 | 5109C1 | 23518770 | Tobacco |
| PsROMT-L | 574 | 5109C6 | 23653450 | Tobacco |
| PsROMT-L | 200 | 5110E11 | 13610509 | Tobacco |
| PsROMT-L | 281 | 532C11 | 23364997 | Tobacco |
| PsROMT-L | 235 | 531A10 | 23365150 | Tobacco |
| PsROMT-L | 792 | 552G11 | 23365746 | Tobacco |
| PsROMT-L | 983 | 531D8 | 23366147 | Tobacco |
| PsROMT-L | 260 | 531G6 | 23370190 | Tobacco |
| PsROMT-L | 466 | 555D2 | 23370421 | Tobacco |
| PsROMT-L | 1353 | 553C11 | 23377150 | Tobacco |
| PsROMT-L | 1297 | 555E1 | 23380202 | Tobacco |
| PsROMT-L | 401 | 554C7 | 23389848 | Tobacco |
| PsROMT-L | 1279 | 555C7 | 23390282 | Tobacco |
| PsROMT-L | 1358 | 531B11 | 23402435 | Tobacco |
| PsROMT-L | 246 | 531C11 | 23411827 | Tobacco |
| PsROMT-L | 1369 | 552H6 | 23418435 | Tobacco |
| PsROMT-L | 1374 | 533D7 | 23428062 | Tobacco |
| PsROMT-L | 1068 | 531G7 | 23471864 | Tobacco |
| PsROMT-L | 906 | 531A9 | 23557650 | Tobacco |
| PsROMT-L | 1248 | 552G2 | 23697027 | Tobacco |
| PsROMT-L | 964 | 531D10 | 23746932 | Tobacco |
| PsROMT-L | 766 | 552C7 | 23772039 | Tobacco |
| PsROMT-L | 472 | 555D3 | 23783423 | Tobacco |
| PsROMT-L | 1376 | 531E11 | 23394987 | Tobacco |
| PsROMT-L | 1378 | 553B11 | 23368763 | Tobacco |
| PsROMT-L | 1351 | 553D1 | 23557531 | Tobacco |
| PsROMT-L | 1335 | 553H9 | 23369680 | Tobacco |

Legend:
L = Luciferase
K = Kanamycin (neomycin phosphotransferase)
AtBBE2 = Arabidopsis berberine bridge enzyme gene 2 promoter
AtBBE5 = Arabidopsis berberine bridge enzyme gene 5 promoter
AtCR2 = Arabidopsis putative codeinone reductase gene 2 promoter
AtROX6 = Arabidopsis putative reticuline oxidase gene 6 promoter
AtROX7 = Arabidopsis putative reticuline oxidase gene 7 promoter
EcBBE = Eschscholzia californica berberine bridge enzyme gene promoter
EcNMCH3 = Eschscholzia californica N-methylcoclaurine 3′-hydroxylase gene promoter
AtSS1 = Arabidopsis putative strictosidine synthase gene 1 promoter
AtSS3 = Arabidopsis putative strictosidine synthase gene 3 promoter
AtWDC = Arabidopsis putative tryptophan decarboxylase gene promoter
PsBBE = Papaver somniferum berberine bridge enzyme promoter
PsHMCOMT2 = Papaver somniferum hydroxy N-methyl S-coclaurine 4-O-methyltransferase 2 gene promoter
PsROMT = Papaver somniferum (R,S)-reticuline 7-O-methyltransferase gene promoter

### Example 6

### Determination of Functional Homolog and/or Ortholog Sequences

A subject sequence was considered a functional homolog or ortholog of a query sequence if the subject and query sequences encoded proteins having a similar function and/or activity. A process known as Reciprocal BLAST (Rivera et al., Proc. Natl. Acad. Sci. USA, 95:6239-6244 (1998)) was used to identify potential functional homolog and/or ortholog sequences from databases consisting of all available public and proprietary peptide sequences, including NR from NCBI and peptide translations from Ceres clones.

Before starting a Reciprocal BLAST process, a specific query polypeptide was searched against all peptides from its source species using BLAST in order to identify polypeptides having sequence identity of 80% or greater to the query polypeptide and an alignment length of 85% or greater along the shorter sequence in the alignment. The query polypeptide and any of the aforementioned identified polypeptides were designated as a cluster.

The main Reciprocal BLAST process consists of two rounds of BLAST searches; forward search and reverse search. In the forward search step, a query polypeptide sequence, “polypeptide A,” from source species SA was BLASTed against all protein sequences from a species of interest. Top hits were determined using an E-value cutoff of 10⁻⁵ and an identity cutoff of 35%. Among the top hits, the sequence having the lowest E-value was designated as the best hit, and considered a potential functional homolog or ortholog. Any other top hit that had a sequence identity of 80% or greater to the best hit or to the original query polypeptide was considered a potential functional homolog or ortholog as well. This process was repeated for all species of interest.

In the reverse search round, the top hits identified in the forward search from all species were BLASTed against all protein sequences from the source species SA. A top hit from the forward search that returned a polypeptide from the aforementioned cluster as its best hit was also considered as a potential functional homolog or ortholog.

Functional homologs and/or orthologs were identified by manual inspection of potential functional homolog and/or ortholog sequences. Representative functional homologs and/or orthologs for SEQ ID NO:80, SEQ ID NO:86, SEQ ID NO:95, SEQ ID NO:115, SEQ ID NO:123, SEQ ID NO:141, SEQ ID NO:144, SEQ ID NO:158, SEQ ID NO:168, SEQ ID NO:173, SEQ ID NO:187, SEQ ID NO:200, SEQ ID NO:205, SEQ ID NO:211, SEQ ID NO:216, SEQ ID NO:225, SEQ ID NO:229, SEQ ID NO:235, SEQ ID NO:246, SEQ ID NO:260, SEQ ID NO:264, SEQ ID NO:281, SEQ ID NO:288, SEQ ID NO:301, SEQ ID NO:309, SEQ ID NO:325, SEQ ID NO:333, SEQ ID NO:345, SEQ ID NO:350, SEQ ID NO:356, SEQ ID NO:364, SEQ ID NO:370, SEQ ID NO:376, SEQ ID NO:382, SEQ ID NO:387, SEQ ID NO:392, SEQ ID NO:401, SEQ ID NO:411, SEQ ID NO:419, SEQ ID NO:434, SEQ ID NO:450, SEQ ID NO:458, SEQ ID NO:466, SEQ ID NO:472, SEQ ID NO:494, SEQ ID NO:506, SEQ ID NO:516, SEQ ID NO:523, SEQ ID NO:532, SEQ ID NO:548, SEQ ID NO:565, SEQ ID NO:574, SEQ ID NO:579, SEQ ID NO:593, SEQ ID NO:599, SEQ ID NO:608, SEQ ID NO:613, SEQ ID NO:619, SEQ ID NO:632, SEQ ID NO:639, SEQ ID NO:648, SEQ ID NO:652, SEQ ID NO:659, SEQ ID NO:664, SEQ ID NO:674, SEQ ID NO:686, SEQ ID NO:695, SEQ ID NO:698, SEQ ID NO:703, SEQ ID NO:711, SEQ ID NO:716, SEQ ID NO:721, SEQ ID NO:732, SEQ ID NO:748, SEQ ID NO:760, SEQ ID NO:766, SEQ ID NO:769, SEQ ID NO:777, SEQ ID NO:792, SEQ ID NO:797, SEQ ID NO:812, SEQ ID NO:822, SEQ ID NO:828, SEQ ID NO:834, SEQ ID NO:840, SEQ ID NO:845, SEQ ID NO:851, SEQ ID NO:856, SEQ ID NO:874, SEQ ID NO:889, SEQ ID NO:906, SEQ ID NO:921, SEQ ID NO:931, SEQ ID NO:946, SEQ ID NO:964, SEQ ID NO:973, SEQ ID NO:983, SEQ ID NO:992, SEQ ID NO:1001, SEQ ID NO:1019, SEQ ID NO:1026, SEQ ID NO:1042, SEQ ID NO:1058, SEQ ID NO:1068, SEQ ID NO:1074, SEQ ID NO:1087, SEQ ID NO:1102, SEQ ID NO:1119, SEQ ID NO:1127, SEQ ID NO:1138, SEQ ID NO:1147, SEQ ID NO:1158, SEQ ID NO:1165, SEQ ID NO:1171, SEQ ID NO:1178, SEQ ID NO:1192, SEQ ID NO:1202, SEQ ID NO:1212, SEQ ID NO:1220, SEQ ID NO:1226, SEQ ID NO:1243, SEQ ID NO:1248, SEQ ID NO:1255, SEQ ID NO:1261, SEQ ID NO:1279, SEQ ID NO:1297, SEQ ID NO:1310, SEQ ID NO:1323, SEQ ID NO:1335, SEQ ID NO:1353, SEQ ID NO:1358, SEQ ID NO:1369, SEQ ID NO:1382, SEQ ID NO:1394, SEQ ID NO:1401, SEQ ID NO:1404, SEQ ID NO:1413, SEQ ID NO:1423, SEQ ID NO:1429, and SEQ ID NO:1440 are shown in FIGS. 1-140, respectively. The percent identities of functional homologs and/or orthologs to SEQ ID NO:80, SEQ ID NO:86, SEQ ID NO:95, SEQ ID NO:115, SEQ ID NO:123, SEQ ID NO:141, SEQ ID NO:144, SEQ ID NO:152, SEQ ID NO:158, SEQ ID NO:168, SEQ ID NO:173, SEQ ID NO:187, SEQ ID NO:200, SEQ ID NO:205, SEQ ID NO:211, SEQ ID NO:216, SEQ ID NO:225, SEQ ID NO:229, SEQ ID NO:235, SEQ ID NO:246, SEQ ID NO:260, SEQ ID NO:264, SEQ ID NO:281, SEQ ID NO:288, SEQ ID NO:301, SEQ ID NO:309, SEQ ID NO:325, SEQ ID NO:333, SEQ ID NO:345, SEQ ID NO:350, SEQ ID NO:356, SEQ ID NO:364, SEQ ID NO:370, SEQ ID NO:376, SEQ ID NO:382, SEQ ID NO:387, SEQ ID NO:392, SEQ ID NO:401, SEQ ID NO:411, SEQ ID NO:419, SEQ ID NO:434, SEQ ID NO:450, SEQ ID NO:458, SEQ ID NO:466, SEQ ID NO:472, SEQ ID NO:494, SEQ ID NO:506, SEQ ID NO:516, SEQ ID NO:523, SEQ ID NO:532, SEQ ID NO:548, SEQ ID NO:565, SEQ ID NO:574, SEQ ID NO:579, SEQ ID NO:590, SEQ ID NO:593, SEQ ID NO:599, SEQ ID NO:608, SEQ ID NO:613, SEQ ID NO:619, SEQ ID NO:632, SEQ ID NO:639, SEQ ID NO:648, SEQ ID NO:652, SEQ ID NO:659, SEQ ID NO:664, SEQ ID NO:671, SEQ ID NO:674, SEQ ID NO:679, SEQ ID NO:686, SEQ ID NO:695, SEQ ID NO:698, SEQ ID NO:703, SEQ ID NO:711, SEQ ID NO:716, SEQ ID NO:721, SEQ ID NO:732, SEQ ID NO:748, SEQ ID NO:760, SEQ ID NO:766, SEQ ID NO:769, SEQ ID NO:777, SEQ ID NO:792, SEQ ID NO:797, SEQ ID NO:812, SEQ ID NO:822, SEQ ID NO:828, SEQ ID NO:834, SEQ ID NO:840, SEQ ID NO:845, SEQ ID NO:851, SEQ ID NO:856, SEQ ID NO:871, SEQ ID NO:874, SEQ ID NO:889, SEQ ID NO:906, SEQ ID NO:921, SEQ ID NO:931, SEQ ID NO:946, SEQ ID NO:964, SEQ ID NO:973, SEQ ID NO:983, SEQ ID NO:992, SEQ ID NO:1001, SEQ ID NO:1019, SEQ ID NO:1026, SEQ ID NO:1042, SEQ ID NO:1058, SEQ ID NO:1068, SEQ ID NO:1074, SEQ ID NO:1087, SEQ ID NO:1102, SEQ ID NO:1119, SEQ ID NO:1127, SEQ ID NO:1138, SEQ ID NO:1147, SEQ ID NO:1158, SEQ ID NO:1165, SEQ ID NO:1171, SEQ ID NO:1178, SEQ ID NO:1192, SEQ ID NO:1202, SEQ ID NO:1212, SEQ ID NO:1220, SEQ ID NO:1226, SEQ ID NO:1243, SEQ ID NO:1248, SEQ ID NO:1255, SEQ ID NO:1261, SEQ ID NO:1279, SEQ ID NO:1297, SEQ ID NO:1310, SEQ ID NO:1323, SEQ ID NO:1335, SEQ ID NO:1342, SEQ ID NO:1353, SEQ ID NO:1358, SEQ ID NO:1369, SEQ ID NO:1382, SEQ ID NO:1394, SEQ ID NO:1401, SEQ ID NO:1404, SEQ ID NO:1413, SEQ ID NO:1423, SEQ ID NO:1429, and SEQ ID NO:1440 are shown below in Tables 5-150, respectively.

TABLE 5
Percent identity to Ceres cDNA ID 23798983 (SEQ ID NO: 80)

| | | SEQ ID | | |
|---|---|---|---|---|
| Designation | Species | NO: | % Identity | e-value |
| Ceres CLONE | Triticum aestivum | 81 | 86.2 | 2.10E−97 |
| ID no. 916120 | | | | |
| Ceres CLONE | Glycine max | 82 | 57.4 | 1.89E−48 |
| ID no. 464614 | | | | |
| Public GI no. | Arabidopsis thaliana | 83 | 54.1 | 4.59E−40 |
| 62320596 | | | | |
| Public GI no. | Arabidopsis thaliana | 84 | 53.6 | 1.20E−39 |
| 42566740 | | | | |

TABLE 6
Percent identity to Ceres cDNA ID 23389356 (SEQ ID NO: 86)

| | | SEQ ID | | |
|---|---|---|---|---|
| Designation | Species | NO: | % Identity | e-value |
| Ceres CLONE ID | Zea mays | 87 | 48.9 | 1.90E−27 |
| no. 1446017 | | | | |
| Public GI no. | Oryza sativa subsp. | 88 | 46.5 | 7.19E−26 |
| 53370700 | japonica | | | |
| Ceres CLONE ID | Zea mays | 89 | 43.3 | 1.99E−22 |
| no. 316709 | | | | |
| Ceres CLONE ID | Zea mays | 90 | 42.5 | 6.50E−25 |
| no. 1627559 | | | | |
| Ceres CLONE ID | Zea mays | 91 | 39.7 | 3.90E−27 |
| no. 284127 | | | | |

TABLE 7
Percent identity to Ceres cDNA ID 23693590 (SEQ ID NO: 95)

| | | SEQ ID | % | |
|---|---|---|---|---|
| Designation | Species | NO: | Identity | e-value |
| Public GI no. 1370160 | Lotus japonicus | 96 | 91.9 | 1.10E−102 |
| Public GI no. 560504 | Vicia faba | 97 | 91.9 | 3.70E−102 |
| Public GI no. 541980 | Vicia faba | 98 | 91 | 4.20E−101 |
| Ceres CLONE ID no. 6827 | Arabidopsis thaliana | 99 | 88.2 | 1.00E−97 |
| Public GI no. 5714658 | Gossypium hirsutum | 100 | 86.1 | 1.10E−95 |
| Public GI no. 5714660 | Gossypium hirsutum | 101 | 85.2 | 6.70E−94 |
| Public GI no. 34913324 | Oryza sativa subsp. | 102 | 79 | 1.19E−85 |
| | japonica | | | |
| Ceres CLONE ID no. 221941 | Zea mays | 103 | 78.3 | 7.59E−86 |
| Public GI no. 303730 | Pisum sativum | 104 | 72.1 | 5.70E−65 |
| Public GI no. 218228 | Oryza sativa | 105 | 65 | 9.19E−58 |
| Ceres CLONE ID no. 789317 | Triticum aestivum | 106 | 64.9 | 1.20E−66 |
| Ceres CLONE ID no. 1068093 | Brassica napus | 107 | 64.4 | 7.09E−67 |
| Public GI no. 53792703 | Oryza sativa | 108 | 64.4 | 3.99E−57 |
| Public GI no. 974778 | Beta vulgaris subsp. | 109 | 59.5 | 5.50E−60 |
| | vulgaris | | | |
| Public GI no. 3025293 | Chlamydomonas | 110 | 58.9 | 4.90E−59 |
| | reinhardtii | | | |
| Public GI no. 6688535 | Lycopersicon | 111 | 55.9 | 1.19E−57 |
| | esculentum | | | |

TABLE 8
Percent identity to Ceres cDNA ID 23663607 (SEQ ID NO: 115)

| | | SEQ ID | % | |
|---|---|---|---|---|
| Designation | Species | NO: | Identity | e-value |
| Public | Oryza sativa subsp. | 116 | 51.1 | 2.19E−47 |
| GI no. 34911396 | japonica | | | |
| Public | Arabidopsis thaliana | 117 | 50.3 | 1.39E−63 |
| GI no. 12324210 | | | | |
| Public | Oryza sativa subsp. | 118 | 50 | 1.90E−57 |
| GI no. 56784967 | japonica | | | |
| Public | Oryza sativa subsp. | 119 | 47.5 | 1.20E−48 |
| GI no. 50932649 | japonica | | | |

TABLE 9
Percent identity to Ceres cDNA ID 23522096 (SEQ ID NO: 123)

| | | SEQ ID | | |
|---|---|---|---|---|
| Designation | Species | NO: | % Identity | e-value |
| Public GI no. 30523252 | Brassica oleracea var. | 124 | 51.6 | 2.80E−06 |
| | capitata | | | |
| Ceres CLONE ID no. 244495 | Zea mays | 125 | 50.6 | 3.00E−09 |
| Ceres CLONE ID no. 326824 | Zea mays | 126 | 50.6 | 3.90E−09 |
| Public GI no. 45181459 | Brassica rapa | 127 | 50 | 3.80E−06 |
| Public GI no. 52789958 | Gossypium hirsutum | 128 | 44.9 | 2.09E−06 |
| Public GI no. 82313 | Antirrhinum majus | 129 | 41.5 | 6.19E−07 |
| Public GI no. 20219014 | Lycopersicon | 130 | 41.5 | 8.39E−07 |
| | esculentum | | | |
| Public GI no. 6580941 | Picea abies | 131 | 41.2 | 1.39E−06 |
| Public GI no. 45268960 | Physcomitrella patens | 132 | 40.8 | 8.99E−06 |
| Public GI no. 55792842 | Physalis peruviana | 133 | 40.3 | 1.90E−07 |
| Public GI no. 6580939 | Picea abies | 134 | 40.3 | 3.50E−06 |
| Public GI no. 46917358 | Asparagus virgatus | 135 | 40.1 | 3.70E−07 |
| Public GI no. 30523364 | Brassica rapa | 136 | 39.7 | 2.80E−06 |
| Public GI no. 55792848 | Physalis pubescens | 137 | 39.4 | 1.89E−06 |
| Public GI no. 22091477 | Daucus carota subsp. | 138 | 39.1 | 3.69E−06 |
| | sativus | | | |
| Public GI no. 5031217 | Liquidambar | 139 | 39 | 4.29E−06 |
| | styraciflua | | | |

TABLE 10
Percent identity to Ceres cDNA ID 23447462 (SEQ ID NO: 141)

| | | | % | |
|---|---|---|---|---|
| Designation | Species | SEQ ID NO: | Identity | e-value |
| Public | Oryza sativa subsp. | 142 | 39.2 | 0 |
| GI no. 50923905 | japonica | | | |

TABLE 11
Percent identity to Ceres cDNA ID 23499985 (SEQ ID NO: 144)

| | | SEQ ID | | |
|---|---|---|---|---|
| Designation | Species | NO: | % Identity | e-value |
| Public | Sorghum bicolor | 145 | 47.9 | 2.20E−07 |
| GI no. 1076760 | | | | |
| Public | Sorghum bicolor | 146 | 47.9 | 4.90E−07 |
| GI no. 297482 | | | | |
| Public | Hordeum vulgare | 147 | 46.5 | 4.39E−06 |
| GI no. 1869928 | subsp. vulgare | | | |
| Ceres CLONE ID | Zea mays | 148 | 43.8 | 1.90E−07 |
| no. 986028 | | | | |
| Public | Oryza sativa subsp. | 149 | 43.6 | 9.30E−10 |
| GI no. 12039274 | japonica | | | |
| Public | Coix lacryma-jobi | 150 | 43 | 8.50E−07 |
| GI no. 463212 | | | | |

TABLE 12
Percent identity to Ceres cDNA ID 23651179 (SEQ ID NO: 152)

| | | SEQ ID | % | |
|---|---|---|---|---|
| Designation | Species | NO: | Identity | e-value |
| Public GI no. | Arabidopsis thaliana | 153 | 97.9 | 5.40E−124 |
| 29027741 | | | | |
| Public GI no. | Arabidopsis thaliana | 154 | 97.5 | 4.90E−123 |
| 29027743 | | | | |
| Public GI no. | Arabidopsis thaliana | 155 | 96.6 | 3.40E−122 |
| 29027745 | | | | |
| Public GI no. | Arabidopsis thaliana | 156 | 96.2 | 7.10E−122 |
| 29027747 | | | | |

TABLE 13
Percent identity to Ceres cDNA ID 24374230 (SEQ ID NO: 158)

| | | SEQ ID | % | |
|---|---|---|---|---|
| Designation | Species | NO: | Identity | e-value |
| Ceres CLONE ID no. 1507510 | Glycine max | 159 | 91.3 | 0 |
| Ceres CLONE ID no. 602357 | Glycine max | 160 | 77.3 | 4.70E−118 |
| Ceres CLONE ID no. 557575 | Glycine max | 161 | 77 | 1.50E−119 |
| Ceres CLONE ID no. 1119778 | Glycine max | 162 | 78.5 | 1.40E−52 |
| Public GI no. 50931081 | Oryza sativa subsp. | 163 | 71 | 9.10E−106 |
| | japonica | | | |
| Ceres CLONE ID no. 500887 | Zea mays | 164 | 70.1 | 3.90E−105 |
| Ceres CLONE ID no. 221299 | Zea mays | 165 | 68.1 | 2.79E−104 |
| Ceres CLONE ID no. 702388 | Triticum aestivum | 166 | 66.9 | 2.10E−99 |

TABLE 14
Percent identity to Ceres cDNA ID 23547976 (SEQ ID NO: 168)

| | | SEQ ID | % | |
|---|---|---|---|---|
| Designation | Species | NO: | Identity | e-value |
| Ceres CLONE | Zea mays | 169 | 48.9 | 4.69E−08 |
| ID no. 1358913 | | | | |
| Public GI | Thellungiella halophila | 170 | 48.9 | 6.69E−07 |
| no. 20340241 | | | | |
| Public GI | Hevea brasiliensis | 171 | 44.8 | 8.79E−07 |
| no. 37901055 | | | | |

TABLE 15
Percent identity to Ceres cDNA ID 13653045 (SEQ ID NO: 173)

| | | SEQ ID | % | |
|---|---|---|---|---|
| Designation | Species | NO: | Identity | e-value |
| Public GI no. 11385590 | Nicotiana tabacum | 174 | 70.6 | 1.20E−117 |
| Public GI no. 11385596 | Nicotiana tabacum | 175 | 70.1 | 4.20E−117 |
| Public GI no. 57899209 | Oryza sativa subsp. | 176 | 69.3 | 2.80E−72 |
| | japonica | | | |
| Ceres CLONE ID no. 1563222 | Zea mays | 177 | 67.2 | 1.39E−107 |
| Public GI no. 11385602 | Nicotiana tabacum | 178 | 63.8 | 4.10E−103 |
| Public GI no. 38564733 | Helianthus annuus | 179 | 33.7 | 6.60E−13 |
| Public GI no. 11385590_T | Artificial Sequence | 180 | 69.9 | 1.00E−131 |
| Public GI no. 11385596_T | Artificial Sequence | 181 | 69.5 | 1.00E−130 |
| Public GI no. 57899209_T | Artificial Sequence | 182 | 54 | 3.00E−88 |
| Ceres CLONE ID no. 1563222_T | Artificial Sequence | 183 | 65.3 | 1.00E−120 |
| Public GI no. 11385602_T | Artificial Sequence | 184 | 67.9 | 1.00E−115 |
| Public GI no. 38564733_T | Artificial Sequence | 185 | 26.4 | 3.00E−18 |

TABLE 16
Percent identity to Ceres cDNA ID 23477523 (SEQ ID NO: 187)

| | | SEQ ID | % | |
|---|---|---|---|---|
| Designation | Species | NO: | Identity | e-value |
| Public GI no. 9967526 | Brassica oleracea | 188 | 81.4 | 1.00E−113 |
| | var. capitata | | | |
| Public GI no. 50511733 | Brassica napus | 189 | 80.8 | 4.49E−113 |
| Public GI no. 50511731 | Brassica rapa subsp. | 190 | 80.5 | 6.70E−110 |
| | pekinensis | | | |
| Public GI no. 50511725 | rassica rapa subsp. | 191 | 80.5 | 1.39E−109 |
| | chinensis | | | |
| Public GI no. 50511729 | Brassica oleracea | 192 | 80.4 | 5.09E−112 |
| | var. capitata | | | |
| Public GI no. 50511727 | Brassica oleracea | 193 | 78.9 | 5.90E−111 |
| | var. capitata | | | |
| Public GI no. 27262829 | Brassica rapa subsp. rapa | 194 | 78.7 | 4.39E−106 |
| Public GI no. 27262839 | Brassica oleracea | 195 | 78.5 | 6.70E−110 |
| | var. gongylodes | | | |
| Public GI no. 27262831 | Brassica oleracea | 196 | 78.5 | 8.59E−110 |
| | var. acephala | | | |
| Public GI no. 27262837 | Brassica oleracea | 197 | 78.1 | 2.89E−109 |
| | var. italica | | | |
| Public GI no. 27262833 | Brassica oleracea | 198 | 77.8 | 2.29E−109 |
| | var. botrytis | | | |

TABLE 17
Percent identity to Ceres cDNA ID 13610509 (SEQ ID NO: 200)

| | | SEQ ID | % | |
|---|---|---|---|---|
| Designation | Species | NO: | Identity | e-value |
| Ceres CLONE ID no. 514234 | Glycine max | 201 | 53.6 | 1.00E−112 |
| Public GI no. 66947626 | Medicago truncatula | 202 | 51.9 | 1.00E−112 |
| Ceres CLONE ID no. 324706 | Zea mays | 203 | 38.3 | 1.00E−72 |

TABLE 18
Percent identity to Ceres cDNA ID 23503364 (SEQ ID NO: 205)

| | | SEQ ID | % | |
|---|---|---|---|---|
| Designation | Species | NO: | Identity | e-value |
| Ceres CLONE ID no. 475115 | Glycine max | 206 | 59.7 | 0 |
| Ceres CLONE ID no. 925463 | Triticum aestivum | 207 | 49.2 | 3.89E−10 |
| Public GI no. 34902824 | Oryza sativa subsp. | 208 | 51.4 | 8.19E−105 |
| | japonica | | | |
| Ceres CLONE ID no. 281953 | Zea mays | 209 | 48.2 | 7.80E−100 |

TABLE 19
Percent identity to Ceres cDNA ID 12676498 (SEQ ID NO: 211)

| | | SEQ ID | % | |
|---|---|---|---|---|
| Designation | Species | NO: | Identity | e-value |
| Public GI no. 34895192 | Oryza sativa | 212 | 61.3 | 0 |
| | subsp. japonica | | | |
| Public GI no. 2959360 | Zea mays | 213 | 61.3 | 0 |
| Public GI no. 53792821 | Oryza sativa | 214 | 61.28 | 0 |
| | subsp. japonica | | | |

TABLE 20
Percent identity to Ceres cDNA ID 4984839 (SEQ ID NO: 216)

| | | SEQ ID | % | |
|---|---|---|---|---|
| Designation | Species | NO: | Identity | e-value |
| Public GI no. 31580813 | Brassica napus | 217 | 62 | 1.60E−46 |
| Public GI no. 17933458 | Brassica napus | 218 | 60.5 | 6.20E−45 |
| Public GI no. 17933450 | Brassica napus | 219 | 59.6 | 2.29E−45 |
| Ceres CLONE ID no. 1065387 | Brassica napus | 220 | 59.5 | 1.79E−45 |
| Public GI no. 17933456 | Brassica napus | 221 | 59.4 | 7.10E−44 |
| Ceres CLONE ID no. 1091989 | Brassica napus | 222 | 59.4 | 7.10E−44 |
| Public GI no. 30523252 | Brassica oleracea | 223 | 58.6 | 2.09E−44 |
| | var. capitata | | | |

TABLE 21
Percent identity to Ceres cDNA ID 23544026 (SEQ ID NO: 225)

| | | SEQ ID | % | |
|---|---|---|---|---|
| Designation | Species | NO: | Identity | e-value |
| Ceres CLONE ID no. 2553 | Arabidopsis thaliana | 226 | 98.5 | 0 |
| Ceres CLONE ID no. 659863 | Glycine max | 227 | 55.4 | 3.00E−75 |

TABLE 22
Percent identity to Ceres cDNA ID 13579142 (SEQ ID NO: 229)

| | | SEQ ID | % | |
|---|---|---|---|---|
| Designation | Species | NO: | Identity | e-value |
| Ceres CLONE ID no. 463860 | Glycine max | 230 | 57.9 | 5.00E−98 |
| Public GI no. 50927857 | Oryza sativa subsp. | 231 | 54.9 | 2.00E−89 |
| | japonica | | | |
| Ceres CLONE ID no. 296774 | Zea mays | 232 | 51.7 | 2.00E−83 |
| Ceres CLONE ID no. 843076 | Triticum aestivum | 233 | 54.4 | 5.00E−85 |

TABLE 23
Percent identity to Ceres cDNA ID 23365150 (SEQ ID NO: 235)

| | | SEQ ID | % | |
|---|---|---|---|---|
| Designation | Species | NO: | Identity | e-value |
| Public GI no. 4996642 | Oryza sativa subsp. | 236 | 63.8 | 2.00E−36 |
| | japonica | | | |
| Public GI no. 50253202 | Oryza sativa subsp. | 237 | 63.8 | 4.09E−36 |
| | japonica | | | |
| Public GI no. 47900733 | Solanum demissum | 238 | 61.2 | 1.30E−33 |
| Public GI no. 7489820 | Zea mays | 239 | 59.5 | 1.59E−31 |
| Public GI no. 4996644 | Oryza sativa subsp. | 240 | 56.9 | 2.49E−29 |
| | japonica | | | |
| Public GI no. 37051125 | Pisum sativum | 241 | 52.5 | 9.29E−39 |
| Ceres CLONE ID no. 543840 | Glycine max | 242 | 52.4 | 1.10E−36 |
| Public GI no. 33332411 | Oryza sativa | 243 | 49.4 | 4.69E−31 |
| Public GI no. 42556524 | Triticum aestivum | 244 | 46 | 4.69E−31 |

TABLE 24
Percent identity to Ceres cDNA ID 23411827 (SEQ ID NO: 246)

| | | SEQ ID | % | |
|---|---|---|---|---|
| Designation | Species | NO: | Identity | e-value |
| Public GI no. 20259679 | Arabidopsis thaliana | 247 | 61.5 | 1.10E−100 |
| Public GI no. 25354653 | Arabidopsis thaliana | 248 | 61.5 | 9.30E−97 |
| Public GI no. 34900512 | Oryza sativa subsp. | 249 | 58.8 | 3.50E−81 |
| | japonica | | | |
| Public GI no. 51100730 | Ipomoea nil | 250 | 58.2 | 8.29E−96 |
| Public GI no. 46395277 | Pinus thunbergii | 251 | 58.1 | 6.60E−80 |
| Ceres CLONE ID no. 374770 | Zea mays | 252 | 54 | 5.10E−80 |
| Public GI no. 5081557 | Petunia x hybrida | 253 | 52.9 | 4.99E−89 |
| Public GI no. 53830033 | Triticum urartu | 254 | 52.3 | 4.50E−81 |
| Public GI no. 53801434 | Triticum monococcum | 255 | 52.3 | 1.90E−80 |
| Public GI no. 53830021 | Triticum aestivum | 256 | 52.2 | 9.30E−81 |
| | subsp. spelta | | | |
| Public GI no. 53830029 | Triticum aestivum | 257 | 52 | 9.30E−81 |
| Public GI no. 53830035 | Triticum turgidum | 258 | 51.8 | 2.50E−80 |
| | subsp. carthlicum | | | |

TABLE 25
Percent identity to Ceres cDNA ID 23370190 (SEQ ID NO: 260)

| | | SEQ ID | % | |
|---|---|---|---|---|
| Designation | Species | NO: | Identity | e-value |
| Ceres CLONE ID no. 287298 | Zea mays | 261 | 67.5 | 7.60E−61 |
| Ceres CLONE ID no. 533616 | Glycine max | 262 | 63 | 2.79E−63 |
| Public GI no. 38196013 | Brassica oleracea | 1476 | 66.3 | 1.10E−40 |
| Public GI no. 60460512 | Gossypium hirsutum | 1477 | 63.8 | 1.00E−49 |
| Public GI no. 38260661 | Arabidopsis pumila | 1478 | 63.7 | 1.40E−40 |
| Ceres CLONE ID no. 1242254 | Glycine max | 1479 | 61.7 | 2.90E−61 |
| Public GI no. 38260624 | Arabidopsis arenosa | 1480 | 58.2 | 1.40E−40 |
| Public GI no. 34906436 | Oryza sativa subsp. | 1481 | 57.8 | 1.30E−65 |
| | japonica | | | |
| Public GI no. 56605376 | Cucumis sativus | 1482 | 56.2 | 6.40E−66 |
| Ceres CLONE ID no. 673872 | Glycine max | 1483 | 56.1 | 2.39E−82 |
| Ceres CLONE ID no. 997341 | Zea mays | 1484 | 50.5 | 2.10E−42 |

TABLE 26
Percent identity to Ceres cDNA ID 23367111 (SEQ ID NO: 264)

| | | SEQ ID | % | |
|---|---|---|---|---|
| Designation | Species | NO: | Identity | e-value |
| Public GI no. 55585713 | Nicotiana tabacum | 265 | 82.8 | 7.59E−30 |
| Public GI no. 30526297 | Lycopersicon | 266 | 75.2 | 4.19E−31 |
| | esculentum | | | |
| Public GI no. 57012875 | Nicotiana sylvestris | 267 | 74.1 | 3.00E−32 |
| Public GI no. 57012757 | Nicotiana tabacum | 268 | 74.1 | 3.29E−31 |
| Ceres CLONE ID no. 953351 | Brassica napus | 269 | 74 | 2.40E−50 |
| Public GI no. 4099914 | Stylosanthes hamata | 270 | 73 | 9.29E−35 |
| Public GI no. 50931913 | Oryza sativa subsp. | 271 | 72.6 | 6.79E−31 |
| | japonica | | | |
| Public GI no. 4099921 | Stylosanthes hamata | 272 | 72 | 5.49E−33 |
| Public GI no. 37625035 | Vitis aestivalis | 273 | 71.4 | 9.99E−36 |
| Ceres CLONE ID no. 326267 | Zea mays | 274 | 71.2 | 1.29E−30 |
| Public GI no. 28274832 | Lycopersicon | 275 | 69.6 | 6.79E−31 |
| | esculentum | | | |
| Public GI no. 55824383 | Cucumis sativus | 276 | 68.1 | 3.00E−34 |
| Ceres CLONE ID no. 554848 | Glycine max | 277 | 65.5 | 4.80E−32 |
| Public GI no. 55419650 | Gossypium hirsutum | 278 | 63.8 | 8.39E−32 |
| Ceres CLONE ID no. 280241 | Zea mays | 279 | 61.1 | 2.59E−31 |

TABLE 27
Percent identity to Ceres cDNA ID 23364997 (SEQ ID NO: 281)

| | | SEQ ID | % | |
|---|---|---|---|---|
| Designation | Species | NO: | Identity | e-value |
| Public GI no. 11994583 | Arabidopsis thaliana | 282 | 96.7 | 1.30E−99 |
| Ceres CLONE ID no. 1021269 | Triticum aestivum | 283 | 58.5 | 1.70E−42 |
| Ceres CLONE ID no. 592400 | Glycine max | 284 | 57.8 | 4.90E−59 |
| Ceres CLONE ID no. 302213 | Zea mays | 285 | 53.8 | 3.09E−41 |
| Public GI no. 50900102 | Oryza sativa subsp. | 286 | 51.9 | 1.89E−41 |
| | japonica | | | |

TABLE 28
Percent identity to Ceres cDNA ID 23376150 (SEQ ID NO: 288)

| | | SEQ ID | % | |
|---|---|---|---|---|
| Designation | Species | NO: | Identity | e-value |
| Public GI no. 32362301 | Arabidopsis thaliana | 289 | 99 | 0 |
| Public GI no. 8569103 | Arabidopsis thaliana | 290 | 98.6 | 0 |
| Ceres CLONE ID no. 597353 | Glycine max | 291 | 62.2 | 4.30E−83 |
| Ceres CLONE ID no. 244954 | Zea mays | 292 | 54.6 | 1.49E−64 |
| Public GI no. 34105723 | Zea mays | 293 | 54.6 | 5.19E−71 |
| Public GI no. 34105719 | Zea mays | 294 | 54 | 1.49E−64 |
| Public GI no. 34912214 | Oryza sativa subsp. | 295 | 53.7 | 1.00E−67 |
| | japonica | | | |
| Ceres CLONE ID no. 292556 | Zea mays | 296 | 53.2 | 2.40E−66 |
| Public GI no. 33286863 | Zea mays | 297 | 53.2 | 2.40E−66 |
| Ceres CLONE ID no. 241094 | Zea mays | 298 | 52.7 | 6.40E−66 |
| Ceres CLONE ID no. 727806 | Triticum aestivum | 299 | 52.5 | 4.80E−68 |

TABLE 29
Percent identity to Ceres cDNA ID 23649144 (SEQ ID NO: 301)

| | | SEQ ID | % | |
|---|---|---|---|---|
| Designation | Species | NO: | Identity | e-value |
| Public GI no. 22137220 | Arabidopsis thaliana | 302 | 98.9 | 0 |
| Ceres CLONE ID no. 460973 | Zea mays | 303 | 75.7 | 6.29E−114 |
| Ceres CLONE ID no. 464226 | Glycine max | 304 | 75.6 | 3.49E−113 |
| Public GI no. 50915436 | Oryza sativa subsp. | 305 | 74.8 | 1.30E−108 |
| | japonica | | | |
| Ceres CLONE ID no. 1069366 | Glycine max | 306 | 73.4 | 6.89E−108 |
| Public GI no. 50915434 | Oryza sativa subsp. | 307 | 63.8 | 1.10E−79 |
| | japonica | | | |

TABLE 30
Percent identity to Ceres cDNA ID 23370269 (SEQ ID NO: 309)

| | | SEQ ID | % | |
|---|---|---|---|---|
| Designation | Species | NO: | Identity | e-value |
| Ceres CLONE ID no. 38635 | Arabidopsis thaliana | 310 | 86.6 | 3.60E−118 |
| Public GI no. 21593407 | Arabidopsis thaliana | 311 | 86.6 | 3.60E−118 |
| Public GI no. 28827386 | Arabidopsis thaliana | 312 | 86.2 | 9.70E−118 |
| Ceres CLONE ID no. 1375513 | Zea mays | 313 | 86.1 | 6.49E−112 |
| Ceres CLONE ID no. 1242841 | Glycine max | 314 | 82.6 | 4.80E−116 |
| Public GI no. 12651665 | Medicago sativa | 315 | 81.4 | 7.49E−111 |
| Public GI no. 14192880 | Oryza sativa | 316 | 74.5 | 5.30E−94 |
| Public GI no. 50939155 | Oryza sativa subsp. | 317 | 74.5 | 5.30E−94 |
| | japonica | | | |
| Ceres CLONE ID no. 1063922 | Zea mays | 318 | 74.1 | 2.70E−99 |
| Public GI no. 62701860 | Oryza sativa subsp. | 319 | 70.7 | 1.60E−85 |
| | japonica | | | |
| Ceres CLONE ID no. 293659 | Zea mays | 320 | 70.3 | 3.19E−94 |
| Ceres CLONE ID no. 1372772 | Zea mays | 321 | 69.1 | 3.00E−91 |
| Ceres CLONE ID no. 262186 | Zea mays | 322 | 69.1 | 4.10E−94 |
| Ceres CLONE ID no. 484170 | Zea mays | 323 | 68.3 | 2.59E−92 |

TABLE 31
Percent identity to Ceres cDNA ID 23420310 (SEQ ID NO: 325)

| | | SEQ ID | % | |
|---|---|---|---|---|
| Designation | Species | NO: | Identity | e-value |
| Public GI no. 10177159 | Arabidopsis thaliana | 326 | 84.2 | 5.30E−46 |
| Ceres CLONE ID no. 853230 | Glycine max | 327 | 68 | 2.60E−14 |
| Public GI no. 57899525 | Oryza sativa subsp. | 328 | 56.6 | 8.79E−21 |
| | japonica | | | |
| Public GI no. 34897256 | Oryza sativa subsp. | 329 | 53.7 | 3.30E−21 |
| | japonica | | | |
| Ceres CLONE ID no. 892520 | Triticum aestivum | 330 | 53.3 | 3.20E−16 |
| Ceres CLONE ID no. 303140 | Zea mays | 331 | 48.6 | 6.19E−18 |

TABLE 32
Percent identity to Ceres cDNA ID 23764087 (SEQ ID NO: 333)

| | | SEQ ID | % | |
|---|---|---|---|---|
| Designation | Species | NO: | Identity | e-value |
| Public GI no. 34910442 | Oryza sativa subsp. | 334 | 94.23 | 0 |
| | japonica | | | |
| Public GI no. 45510867 | Triticum aestivum | 335 | 92.61 | 0 |
| Public GI no. 8777442 | Arabidopsis thaliana | 336 | 78.85 | 0 |
| Ceres CLONE ID no. 36525 | Arabidopsis thaliana | 337 | 75.7 | 0 |
| Public GI no. 13924514 | Arabidopsis thaliana | 338 | 75.7 | 0 |
| Ceres CLONE ID no. 1242960 | Glycine max | 339 | 73.15 | 0 |
| Public GI no. 6635379 | Brassica oleracea | 340 | 70.64 | 2.89E−115 |
| Ceres CLONE ID no. 530281 | Glycine max | 341 | 70.56 | 0 |
| Public GI no. 7484992 | Arabidopsis thaliana | 342 | 61.14 | 1.19E−102 |
| Public GI no. 13924516 | Arabidopsis thaliana | 343 | 57.44 | 0 |

TABLE 33
Percent identity to Ceres cDNA ID 23460392 (SEQ ID NO: 345)

| | | SEQ ID | % | |
|---|---|---|---|---|
| Designation | Species | NO: | Identity | e-value |
| Public GI no. 51971865 | Arabidopsis thaliana | 346 | 84.5 | 1.50E−57 |
| Public GI no. 7268798 | Triticum aestivum | 347 | 87.9 | 1.40E−38 |
| Ceres CLONE ID no. 783489 | Arabidopsis thaliana | 348 | 43.5 | 1.09E−13 |

TABLE 34
Percent identity to Ceres cDNA ID 23419606 (SEQ ID NO: 350)

| | | SEQ ID | % | |
|---|---|---|---|---|
| Designation | Species | NO: | Identity | e-value |
| Ceres CLONE ID no. 965028 | Brassica napus | 351 | 97.1 | 1.79E−109 |
| Ceres CLONE ID no. 2347 | Arabidopsis thaliana | 352 | 79.6 | 2.39E−82 |
| Public GI no. 21592411 | Arabidopsis thaliana | 353 | 79.6 | 2.39E−82 |
| Public GI no. 21387011 | Arabidopsis thaliana | 354 | 79.1 | 1.69E−81 |

TABLE 35
Percent identity to Ceres cDNA ID 23740209 (SEQ ID NO: 356)

| | | SEQ ID | % | |
|---|---|---|---|---|
| Designation | Species | NO: | Identity | e-value |
| Public GI no. 50940237 | Oryza sativa subsp. | 357 | 69.4 | 4.60E−47 |
| | japonica | | | |
| Ceres CLONE ID no. 617111 | Triticum aestivum | 358 | 62.2 | 7.20E−42 |
| Ceres CLONE ID no. 207075 | Arabidopsis thaliana | 359 | 53.2 | 8.29E−25 |
| Public GI no. 21554154 | Arabidopsis thaliana | 360 | 53.2 | 8.29E−25 |
| Public GI no. 9759080 | Arabidopsis thaliana | 361 | 51.4 | 5.59E−19 |
| Ceres CLONE ID no. 471377 | Glycine max | 362 | 49 | 2.60E−28 |

TABLE 36
Percent identity to Ceres cDNA ID 23374089 (SEQ ID NO: 364)

| | | SEQ ID | % | |
|---|---|---|---|---|
| Designation | Species | NO: | Identity | e-value |
| Public GI no. 50726625 | Oryza sativa subsp. | 365 | 51.5 | 4.49E−49 |
| | japonica | | | |
| Ceres CLONE ID no. 755158 | Triticum aestivum | 366 | 50.4 | 3.00E−36 |

TABLE 37
Percent identity to Ceres cDNA ID 23666854 (SEQ ID NO: 370)

| | | SEQ ID | % | |
|---|---|---|---|---|
| Designation | Species | NO: | Identity | e-value |
| Ceres CLONE ID no. 480900 | Glycine max | 371 | 98.9 | 4.00E−96 |
| Ceres CLONE ID no. 652078 | Glycine max | 372 | 89.2 | 1.60E−85 |
| Public GI no. 22136722 | Arabidopsis thaliana | 373 | 62.7 | 7.59E−47 |
| Public GI no. 7578881 | Spinacia oleracea | 374 | 60.8 | 1.89E−48 |

TABLE 38
Percent identity to Ceres cDNA ID 23662829 (SEQ ID NO: 376)

| | | SEQ ID | % | |
|---|---|---|---|---|
| Designation | Species | NO: | Identity | e-value |
| Ceres CLONE ID no. 12573 | Arabidopsis thaliana | 377 | 63.5 | 6.60E−80 |
| Public GI no. 21537266 | Arabidopsis thaliana | 378 | 63.5 | 6.60E−80 |
| Public GI no. 7269949 | Arabidopsis thaliana | 379 | 63.5 | 6.60E−80 |
| Ceres CLONE ID no. 246144 | Zea mays | 380 | 59.5 | 2.30E−70 |

TABLE 39
Percent identity to Ceres cDNA ID 23698996 (SEQ ID NO: 382)

| | | SEQ ID | % | |
|---|---|---|---|---|
| Designation | Species | NO: | Identity | e-value |
| Public GI no. 50906419 | Oryza sativa subsp. | 383 | 57.9 | 1.20E−69 |
| | japonica | | | |
| Public GI no. 15220810 | Arabidopsis thaliana | 384 | 55.3 | 7.50E−79 |
| Ceres CLONE ID no. 275358 | Zea mays | 385 | 52.8 | 4.29E−76 |

TABLE 40
Percent identity to Ceres cDNA ID 23369491 (SEQ ID NO: 387)

| | | SEQ ID | % | |
|---|---|---|---|---|
| Designation | Species | NO: | Identity | e-value |
| Ceres CLONE ID no. 463738 | Glycine max | 388 | 84.5 | 0 |
| Public GI no. 50923675 | Oryza sativa subsp. | 389 | 80.7 | 0 |
| | japonica | | | |
| Ceres CLONE ID no. 1213577 | Zea mays | 390 | 78.9 | 0 |

TABLE 41
Percent identity to Ceres cDNA ID 23384563 (SEQ ID NO: 392)

| | | SEQ ID | % | |
|---|---|---|---|---|
| Designation | Species | NO: | Identity | e-value |
| Ceres CLONE ID no. 14909 | Arabidopsis thaliana | 393 | 98.8 | 9.89E−93 |
| Ceres CLONE ID no. 33126 | Arabidopsis thaliana | 394 | 98.8 | 2.59E−92 |
| Ceres CLONE ID no. 1338585 | Arabidopsis thaliana | 395 | 98.8 | 2.59E−92 |
| Public GI no. 39653273 | Medicago sativa | 396 | 98.8 | 3.29E−92 |
| Ceres CLONE ID no. 276776 | Zea mays | 397 | 98.3 | 2.59E−92 |
| Ceres CLONE ID no. 1535974 | Zea mays | 398 | 98.3 | 3.29E−92 |
| Ceres CLONE ID no. 240510 | Zea mays | 399 | 98.3 | 2.59E−92 |

TABLE 42
Percent identity to Ceres cDNA ID 23389848 (SEQ ID NO: 401)

| | | SEQ ID | % | |
|---|---|---|---|---|
| Designation | Species | NO: | Identity | e-value |
| Ceres CLONE ID no. 1388526 | Zea mays | 402 | 89.1 | 1.20E−110 |
| Public GI no. 55775124 | Oryza sativa subsp. | 403 | 66.1 | 3.60E−72 |
| | japonica | | | |
| Ceres CLONE ID no. 477450 | Glycine max | 404 | 65.2 | 7.99E−75 |
| Public GI no. 34897896 | Oryza sativa subsp. | 405 | 64.6 | 2.59E−69 |
| | japonica | | | |
| Ceres CLONE ID no. 700178 | Triticum aestivum | 406 | 56.2 | 4.20E−62 |
| Public GI no. 48209876 | Solanum demissum | 407 | 44.3 | 6.89E−37 |
| Public GI no. 48209951 | Solanum demissum | 408 | 44.3 | 8.80E−37 |
| Public GI no. 48057564 | Solanum demissum | 409 | 43.9 | 1.10E−36 |

TABLE 43
Percent identity to Ceres cDNA ID 23384591 (SEQ ID NO: 411)

| | | SEQ ID | % | |
|---|---|---|---|---|
| Designation | Species | NO: | Identity | e-value |
| Public GI no. 9663025 | Arabidopsis thaliana | 412 | 98.5 | 0 |
| Ceres CLONE ID no. 305349 | Zea mays | 413 | 58.1 | 2.10E−60 |
| Ceres CLONE ID no. 220215 | Zea mays | 414 | 56.4 | 2.10E−58 |
| Public GI no. 50945933 | Oryza sativa subsp. | 415 | 53.8 | 5.59E−58 |
| | japonica | | | |
| Public GI no. 52077258 | Oryza sativa subsp. | 416 | 51.8 | 1.39E−61 |
| | japonica | | | |
| Ceres CLONE ID no. 246718 | Zea mays | 417 | 46.7 | 2.80E−49 |

TABLE 44
Percent identity to Ceres cDNA ID 23382112 (SEQ ID NO: 419)

| | | SEQ ID | % | |
|---|---|---|---|---|
| Designation | Species | NO: | Identity | e-value |
| Public GI no. 15293163 | Arabidopsis thaliana | 420 | 98.5 | 0 |
| Public GI no. 34902154 | Oryza sativa subsp. | 421 | 74.2 | 1.99E−62 |
| | japonica | | | |
| Ceres CLONE ID no. 363807 | Zea mays | 422 | 72.8 | 9.99E−59 |
| Public GI no. 62546183 | Glycine max | 423 | 70.8 | 2.39E−82 |
| Public GI no. 15148914 | Phaseolus vulgaris | 424 | 70.4 | 1.90E−80 |
| Public GI no. 56744294 | Solanum demissum | 425 | 68.6 | 1.40E−79 |
| Public GI no. 51871853 | Solanum tuberosum | 426 | 67.9 | 2.50E−55 |
| Public GI no. 53749460 | Solanum demissum | 427 | 66.6 | 3.19E−80 |
| Public GI no. 56785066 | Oryza sativa | 428 | 62.5 | 2.20E−56 |
| Public GI no. 51702424 | Triticum aestivum | 429 | 62.5 | 4.59E−63 |
| Public GI no. 52353038 | Lycopersicon | 430 | 61.8 | 8.60E−60 |
| | esculentum | | | |
| Public GI no. 21105748 | Petunia x hybrida | 431 | 58.5 | 1.99E−55 |
| Public GI no. 4218535 | Triticum sp. | 432 | 57 | 1.20E−53 |

TABLE 45
Percent identity to Ceres cDNA ID 23389418 (SEQ ID NO: 434)

| | | SEQ ID | % | |
|---|---|---|---|---|
| Designation | Species | NO: | Identity | e-value |
| Ceres CLONE ID no. 942980 | Brassica napus | 435 | 72.8 | 2.10E−35 |
| Ceres CLONE ID no. 1265097 | Brassica napus | 436 | 69.3 | 5.80E−33 |
| Ceres CLONE ID no. 571184 | Glycine max | 437 | 64.2 | 2.09E−28 |
| Ceres CLONE ID no. 1052457 | Triticum aestivum | 438 | 63.4 | 1.10E−27 |
| Ceres CLONE ID no. 1609912 | Parthenium | 439 | 62.7 | 3.60E−24 |
| | argentatum | | | |
| Ceres CLONE ID no. 323551 | Zea mays | 440 | 59 | 2.69E−19 |
| Public GI no. 57117314 | Populus x canescens | 441 | 53.3 | 2.00E−08 |
| Public GI no. 50928191 | Oryza sativa subsp. | 442 | 53.2 | 4.90E−20 |
| | japonica | | | |
| Public GI no. 50253143 | Oryza sativa subsp. | 443 | 50.5 | 4.39E−19 |
| | japonica | | | |
| Public GI no. 23451086 | Medicago sativa | 444 | 46.6 | 1.49E−10 |
| Public GI no. 38228693 | Fagus sylvatica | 445 | 46.5 | 3.59E−08 |
| Public GI no. 37901055 | Hevea brasiliensis | 446 | 46 | 1.50E−09 |
| Public GI no. 20340241 | Thellungiella | 447 | 42.3 | 5.50E−12 |
| | halophila | | | |
| Public GI no. 20152976 | Hordeum vulgare | 448 | 42.1 | 2.80E−08 |
| | subsp. vulgare | | | |

TABLE 46
Percent identity to Ceres cDNA ID 23374668 (SEQ ID NO: 450)

| | | SEQ ID | % | |
|---|---|---|---|---|
| Designation | Species | NO: | Identity | e-value |
| Public GI no. 10177389 | Arabidopsis thaliana | 451 | 97.3 | 0 |
| Ceres CLONE ID no. 463247 | Glycine max | 452 | 47.9 | 6.09E−61 |
| Public GI no. 53791916 | Oryza sativa subsp. | 453 | 39.2 | 2.09E−51 |
| | japonica | | | |
| Ceres CLONE ID no. 265056 | Zea mays | 454 | 38.7 | 1.79E−45 |
| Ceres CLONE ID no. 336108 | Zea mays | 455 | 38.7 | 1.79E−45 |
| Ceres CLONE ID no. 906800 | Triticum aestivum | 456 | 38.5 | 1.50E−48 |

TABLE 47
Percent identity to Ceres cDNA ID 23365920 (SEQ ID NO: 458)

| | | SEQ ID | % | |
|---|---|---|---|---|
| Designation | Species | NO: | Identity | e-value |
| Public GI no. 5616313 | Pisum sativum | 459 | 47.4 | 7.59E−70 |
| Ceres CLONE ID no. 751992 | Triticum aestivum | 460 | 47.3 | 1.09E−63 |
| Ceres CLONE ID no. 833872 | Triticum aestivum | 461 | 47.2 | 3.49E−65 |
| Public GI no. 62901482 | Oryza sativa subsp. | 462 | 46.1 | 6.40E−66 |
| | japonica | | | |
| Public GI no. 34906988 | Oryza sativa subsp. | 463 | 46 | 3.09E−57 |
| | japonica | | | |
| Ceres CLONE ID no. 1579587 | Zea mays | 464 | 43.6 | 2.30E−68 |

TABLE 48
Percent identity to Ceres cDNA ID 23370421 (SEQ ID NO: 466)

| | | SEQ ID | % | |
|---|---|---|---|---|
| Designation | Species | NO: | Identity | e-value |
| Ceres CLONE ID no. 870962 | Brassica napus | 467 | 81.3 | 5.39E−85 |
| Ceres CLONE ID no. 562536 | Glycine max | 468 | 67.8 | 1.30E−60 |
| Ceres CLONE ID no. 1032823 | Triticum aestivum | 469 | 53.7 | 3.69E−45 |
| Ceres CLONE ID no. 314156 | Zea mays | 470 | 51.2 | 4.10E−39 |

TABLE 49
Percent identity to Ceres cDNA ID 23783423 (SEQ ID NO: 472)

| | | SEQ ID | % | |
|---|---|---|---|---|
| Designation | Species | NO: | Identity | e-value |
| Public GI no. 9367307 | Hordeum vulgare | 473 | 80.6 | 2.30E−93 |
| | subsp. vulgare | | | |
| Public GI no. 62510920 | Oryza sativa subsp. | 474 | 77.6 | 1.20E−94 |
| | japonica | | | |
| Public GI no. 28630957 | Lolium perenne | 475 | 77.6 | 1.20E−87 |
| Public GI no. 6175371 | Oryza sativa | 476 | 77.2 | 4.10E−94 |
| Public GI no. 33309864 | Elaeis guineensis | 477 | 65 | 4.60E−72 |
| Public GI no. 6467974 | Dendrobium grex | 478 | 64.1 | 6.20E−68 |
| | Madame Thong-In | | | |
| Public GI no. 1483232 | Betula pendula | 479 | 63.5 | 1.89E−66 |
| Public GI no. 38229935 | Asarum caudigerum | 480 | 62.6 | 5.70E−65 |
| Ceres CLONE ID no. 510092 | Zea mays | 481 | 61.3 | 9.99E−66 |
| Public GI no. 29372764 | Zea mays | 482 | 61.3 | 9.99E−66 |
| Public GI no. 33355661 | Crocus sativus | 483 | 61.1 | 2.00E−69 |
| Public GI no. 30090030 | Triticum monococcum | 484 | 60.9 | 9.29E−65 |
| Public GI no. 32478105 | Tradescantia | 485 | 60.6 | 2.40E−66 |
| | virginiana | | | |
| Public GI no. 58423002 | Triticum turgidum | 486 | 60.5 | 2.49E−64 |
| Public GI no. 33391153 | Crocus sativus | 487 | 60.4 | 4.49E−65 |
| Public GI no. 39843110 | Dendrocalamus | 488 | 58.5 | 9.29E−65 |
| | latiflorus | | | |

TABLE 50
Percent identity to Ceres cDNA ID 23538950 (SEQ ID NO: 494)

| | | SEQ ID | % | |
|---|---|---|---|---|
| Designation | Species | NO: | Identity | e-value |
| Ceres CLONE ID no. 111288 | Arabidopsis thaliana | 495 | 94.6 | 1.89E−66 |
| Ceres CLONE ID no. 567184 | Glycine max | 496 | 58.3 | 1.99E−22 |
| Ceres CLONE ID no. 967417 | Brassica napus | 497 | 50 | 8.09E−25 |
| Ceres CLONE ID no. 1360570 | Zea mays | 498 | 49.6 | 1.60E−21 |
| Ceres CLONE ID no. 701370 | Triticum aestivum | 499 | 47.2 | 1.60E−23 |
| Public GI no. 5031281 | Prunus armeniaca | 500 | 47 | 9.90E−23 |
| Public GI no. 35187687 | Oryza sativa subsp. | 501 | 43.6 | 1.99E−20 |
| | indica | | | |
| Ceres CLONE ID no. 849111 | Triticum aestivum | 502 | 42.7 | 6.39E−18 |
| Public GI no. 34910634 | Oryza sativa subsp. | 503 | 40.9 | 1.39E−22 |
| | japonica | | | |
| Ceres CLONE ID no. 1609861 | Parthenium | 504 | 39.3 | 2.80E−17 |
| | argentatum | | | |

TABLE 51
Percent identity to Ceres cDNA ID 24373996 (SEQ ID NO: 506)

| | | SEQ ID | % | |
|---|---|---|---|---|
| Designation | Species | NO: | Identity | e-value |
| Ceres CLONE ID no. 563014 | Glycine max | 507 | 49.7 | 1.29E−49 |
| Public GI no. 22795037 | Populus x canescens | 508 | 48.2 | 1.69E−40 |
| Public GI no. 41059804 | Capsicum annuum | 509 | 47.3 | 2.59E−44 |
| Ceres CLONE ID no. 464515 | Glycine max | 510 | 45.2 | 1.89E−48 |
| Ceres CLONE ID no. 883322 | Triticum aestivum | 511 | 41.9 | 5.29E−30 |
| Ceres CLONE ID no. 244940 | Zea mays | 512 | 40.7 | 1.60E−28 |
| Ceres CLONE ID no. 995691 | Zea mays | 513 | 39.8 | 1.40E−29 |
| Public GI no. 50926652 | Oryza sativa subsp. | 514 | 38.4 | 1.70E−26 |
| | japonica | | | |

TABLE 52
Percent identity to Ceres cDNA ID 23539673 (SEQ ID NO: 516)

| | | SEQ ID | % | |
|---|---|---|---|---|
| Designation | Species | NO: | Identity | e-value |
| Ceres CLONE ID no. 477085 | Glycine max | 517 | 64.8 | 0 |
| Ceres CLONE ID no. 387243 | Zea mays | 518 | 55.1 | 2.40E−130 |
| Ceres CLONE ID no. 379975 | Zea mays | 519 | 54.9 | 0 |
| Public GI no. 50898950 | Oryza sativa subsp. | 520 | 52.2 | 1.79E−125 |
| | japonica | | | |
| Public GI no. 50898952 | Oryza sativa subsp. | 521 | 52.2 | 3.99E−128 |
| | japonica | | | |

TABLE 53
Percent identity to Ceres cDNA ID 23357846 (SEQ ID NO: 523)

| | | SEQ ID | % | |
|---|---|---|---|---|
| Designation | Species | NO: | Identity | e-value |
| Ceres CLONE ID no. 539578 | Glycine max | 524 | 75.5 | 3.79E−29 |
| Ceres CLONE ID no. 596339 | Glycine max | 525 | 74.4 | 9.99E−29 |
| Ceres CLONE ID no. 986002 | Zea mays | 526 | 59.4 | 2.19E−19 |
| Public GI no. 2104677 | Vicia faba | 527 | 53 | 4.69E−22 |
| Public GI no. 23496521 | Lotus japonicus | 528 | 52.4 | 1.09E−22 |
| Public GI no. 6018699 | Lycopersicon | 529 | 52.1 | 1.39E−22 |
| | esculentum | | | |
| Public GI no. 50725042 | Oryza sativa subsp. | 530 | 51.7 | 1.60E−23 |
| | japonica | | | |

TABLE 54
Percent identity to Ceres cDNA ID 12680548 (SEQ ID NO: 532)

| | | SEQ ID | % | |
|---|---|---|---|---|
| Designation | Species | NO: | Identity | e-value |
| Public GI no. 62632894 | Arabidopsis thaliana | 533 | 97.9 | 2.89E−93 |
| Ceres CLONE ID no. 1065387 | Brassica napus | 534 | 87.1 | 1.99E−78 |
| Public GI no. 17933450 | Brassica napus | 535 | 87.1 | 1.99E−78 |
| Public GI no. 31580813 | Brassica napus | 536 | 87 | 7.79E−77 |
| Public GI no. 30523250 | Raphanus sativus | 537 | 85 | 9.20E−74 |
| Public GI no. 30523252 | Brassica oleracea | 538 | 84.6 | 2.10E−76 |
| | var. capitata | | | |
| Ceres CLONE ID no. 963001 | Brassica napus | 539 | 84.6 | 1.10E−75 |
| Public GI no. 30523362 | Brassica napus | 540 | 84.6 | 1.10E−75 |
| Ceres CLONE ID no. 1091989 | Brassica napus | 541 | 84.4 | 4.89E−75 |
| Public GI no. 17933456 | Brassica napus | 542 | 84.4 | 4.89E−75 |
| Public GI no. 30523360 | Brassica rapa | 543 | 84.1 | 6.29E−75 |
| Public GI no. 30523364 | Brassica rapa | 544 | 83.5 | 2.10E−74 |
| Public GI no. 45181459 | Brassica rapa | 545 | 83 | 3.50E−74 |
| Public GI no. 30523366 | Brassica rapa | 546 | 83 | 5.60E−74 |

TABLE 55
Percent identity to Ceres cDNA ID 23357564 (SEQ ID NO: 548)

| | | SEQ ID | % | |
|---|---|---|---|---|
| Designation | Species | NO: | Identity | e-value |
| Ceres CLONE ID no. 11615 | Arabidopsis thaliana | 549 | 90.7 | 2.79E−63 |
| Public GI no. 17104699 | Arabidopsis thaliana | 550 | 90.7 | 2.79E−63 |
| Ceres CLONE ID no. 1027567 | Glycine max | 551 | 90 | 6.79E−62 |
| Ceres CLONE ID no. 1060767 | Zea mays | 552 | 89.3 | 1.39E−61 |
| Ceres CLONE ID no. 1034616 | Brassica napus | 553 | 86.8 | 5.59E−58 |
| Ceres CLONE ID no. 1058733 | Glycine max | 554 | 83.5 | 1.70E−58 |
| Public GI no. 2894109 | Solanum tuberosum | 555 | 74.1 | 2.19E−49 |
| Ceres CLONE ID no. 782784 | Glycine max | 556 | 73.7 | 5.90E−47 |
| Public GI no. 18645 | Glycine max | 557 | 73.7 | 5.90E−47 |
| Ceres CLONE ID no. 721511 | Glycine max | 558 | 73.7 | 7.59E−47 |
| Ceres CLONE ID no. 641329 | Glycine max | 559 | 73.7 | 7.59E−47 |
| Public GI no. 7446213 | Nicotiana tabacum | 560 | 73.3 | 1.60E−46 |
| Public GI no. 1052956 | Ipomoea nil | 561 | 67.1 | 1.79E−45 |

TABLE 56
Percent identity to Ceres cDNA ID 23660778 (SEQ ID NO: 565)

| | | SEQ ID | % | |
|---|---|---|---|---|
| Designation | Species | NO: | Identity | e-value |
| Public GI no. 50251990 | Oryza sativa subsp. | 566 | 46.3 | 1.70E−26 |
| | japonica | | | |
| Ceres CLONE ID no. 304939 | Zea mays | 567 | 49.3 | 1.60E−23 |
| Ceres CLONE ID no. 569545 | Triticum aestivum | 568 | 52.8 | 2.00E−23 |

TABLE 57
Percent identity to Ceres cDNA ID 23653450 (SEQ ID NO: 574)

| | | SEQ ID | % | |
|---|---|---|---|---|
| Designation | Species | NO: | Identity | e-value |
| Public GI no. 50938747 | Oryza sativa subsp. | 575 | 59 | 0 |
| | japonica | | | |
| Ceres CLONE ID no. 458156 | Zea mays | 576 | 58.7 | 0 |
| Ceres CLONE ID no. 918824 | Triticum aestivum | 577 | 55.5 | 3.20E−126 |

TABLE 58
Percent identity to Ceres cDNA ID 23467847 (SEQ ID NO: 579)

| | | SEQ ID | % | |
|---|---|---|---|---|
| Designation | Species | NO: | Identity | e-value |
| Public GI no. 63252923 | Prunus persica | 580 | 42.3 | 3.00E−75 |
| Ceres CLONE ID no. 363807 | Zea mays | 581 | 70.2 | 5.50E−60 |
| Public GI no. 58013003 | Saccharum | 582 | 58.7 | 1.00E−62 |
| | officinarum | | | |
| Public GI no. 52353038 | Lycopersicon | 583 | 66.6 | 2.29E−63 |
| | esculentum | | | |
| Public GI no. 34902154 | Oryza sativa subsp. | 584 | 63.8 | 1.39E−63 |
| | japonica | | | |
| Public GI no. 21105748 | Petunia x hybrida | 585 | 62.4 | 1.80E−54 |
| Public GI no. 66275772 | Triticum aestivum | 586 | 52.7 | 1.00E−70 |
| Public GI no. 53749460 | Solarium demissum | 587 | 58.1 | 8.89E−60 |
| Public GI no. 15148914 | Phaseolus vulgaris | 588 | 56.3 | 2.20E−56 |

TABLE 59
Percent identity to Ceres cDNA ID 23519948 (SEQ ID NO: 590)

| | | SEQ ID | % | |
|---|---|---|---|---|
| Designation | Species | NO: | Identity | e-value |
| Public GI no. 4249383 | Arabidopsis | 591 | 95.4 | 0 |
| | thaliana | | | |

TABLE 60
Percent identity to Ceres cDNA ID 23553534 (SEQ ID NO: 593)

| | | SEQ ID | % | |
|---|---|---|---|---|
| Designation | Species | NO: | Identity | e-value |
| Ceres CLONE ID no. 956332 | Brassica napus | 594 | 96.8 | 9.99E−59 |
| Ceres CLONE ID no. 1049567 | Glycine max | 595 | 80.8 | 3.49E−49 |
| Public GI no. 34898438 | Oryza sativa subsp. | 596 | 75.9 | 2.59E−37 |
| | japonica | | | |
| Ceres CLONE ID no. 280534 | Zea mays | 597 | 72.5 | 7.49E−40 |

TABLE 61
Percent identity to Ceres cDNA ID 23498294 (SEQ ID NO: 599)

| | | SEQ ID | % | |
|---|---|---|---|---|
| Designation | Species | NO: | Identity | e-value |
| Ceres CLONE ID no. 957882 | Brassica napus | 600 | 87 | 1.60E−110 |
| Public GI no. 50726297 | Oryza sativa subsp. | 601 | 65.4 | 2.80E−72 |
| | japonica | | | |
| Ceres CLONE ID no. 739665 | Triticum aestivum | 602 | 62.5 | 5.79E−72 |
| Ceres CLONE ID no. 294374 | Zea mays | 603 | 61.7 | 7.40E−72 |
| Ceres CLONE ID no. 372141 | Zea mays | 604 | 61.2 | 7.59E−70 |
| Ceres CLONE ID no. 656020 | Glycine max | 605 | 56.7 | 1.80E−70 |
| Public GI no. 3334756 | Medicago sativa | 606 | 36.4 | 2.79E−10 |
| | subsp. x varia | | | |

TABLE 62
Percent identity to Ceres cDNA ID 23529931 (SEQ ID NO: 608)

| | | SEQ ID | % | |
|---|---|---|---|---|
| Designation | Species | NO: | Identity | e-value |
| Ceres CLONE ID no. 1021260 | Triticum aestivum | 609 | 75.3 | 9.79E−22 |
| Ceres CLONE ID no. 239775 | Zea mays | 610 | 68.5 | 5.19E−48 |
| Ceres CLONE ID no. 316607 | Zea mays | 611 | 67.9 | 1.39E−47 |

TABLE 63
Percent identity to Ceres cDNA ID 23498685 (SEQ ID NO: 613)

| | | SEQ ID | % | |
|---|---|---|---|---|
| Designation | Species | NO: | Identity | e-value |
| Public GI no. 52077327 | Oryza sativa subsp. | 614 | 67.2 | 2.00E−53 |
| | japonica | | | |
| Ceres CLONE ID no. 1044645 | Glycine max | 615 | 65.8 | 3.70E−54 |
| Ceres CLONE ID no. 1548279 | Zea mays | 616 | 64.4 | 6.90E−53 |
| Ceres CLONE ID no. 727056 | Triticum aestivum | 617 | 62.3 | 1.00E−51 |

TABLE 64
Percent identity to Ceres cDNA ID 23515088 (SEQ ID NO: 619)

| | | SEQ ID | % | |
|---|---|---|---|---|
| Designation | Species | NO: | Identity | e-value |
| Public GI no. 50916012 | Oryza sativa subsp. | 620 | 58.7 | 8.69E−31 |
| | japonica | | | |
| Public GI no. 861091 | Pisum sativum | 621 | 53.6 | 2.19E−40 |
| Public GI no. 2346972 | Petunia x hybrida | 622 | 53.3 | 5.30E−46 |
| Ceres CLONE ID no. 519630 | Glycine max | 623 | 51.4 | 1.70E−49 |
| Public GI no. 7228329 | Medicago sativa | 624 | 45.4 | 2.69E−19 |
| | subsp. x varia | | | |
| Public GI no. 2981169 | Nicotiana tabacum | 625 | 45.1 | 4.09E−16 |
| Public GI no. 55734108 | Catharanthus roseus | 626 | 43 | 1.10E−21 |
| Public GI no. 33331578 | Capsicum annuum | 627 | 41.8 | 1.49E−16 |
| Public GI no. 51871855 | Solanum tuberosum | 628 | 41.8 | 3.69E−10 |
| Public GI no. 2058506 | Brassica rapa | 629 | 40.8 | 8.09E−25 |
| Public GI no. 2058504 | Brassica rapa | 630 | 40.2 | 2.70E−24 |

TABLE 65
Percent identity to Ceres cDNA ID 24375036 (SEQ ID NO: 632)

| | | SEQ ID | % | |
|---|---|---|---|---|
| Designation | Species | NO: | Identity | e-value |
| Ceres CLONE ID no. 971843 | Brassica napus | 633 | 59.47 | 1.19E−38 |
| Ceres CLONE ID no. 361557 | Zea mays | 634 | 28.72 | 5.39E−20 |
| Ceres CLONE ID no. 535370 | Glycine max | 635 | 29.9 | 9.00E−24 |

TABLE 66
Percent identity to Ceres cDNA ID 23544992 (SEQ ID NO: 639)

| | | SEQ ID | % | |
|---|---|---|---|---|
| Designation | Species | NO: | Identity | e-value |
| Public GI no. 1362020 | Arabidopsis thaliana | 640 | 91.6 | 5.89E−95 |
| Public GI no. 51536147 | Oryza sativa subsp. | 641 | 52.8 | 1.09E−20 |
| | japonica | | | |
| Ceres CLONE ID no. 1060169 | Glycine max | 642 | 48.4 | 2.49E−16 |
| Public GI no. 50913293 | Oryza sativa subsp. | 643 | 46.9 | 2.90E−29 |
| | japonica | | | |
| Ceres CLONE ID no. 1461776 | Zea mays | 644 | 46.3 | 2.40E−27 |
| Public GI no. 50946585 | Oryza sativa subsp. | 645 | 43.3 | 4.09E−23 |
| | japonica | | | |
| Public GI no. 18390109 | Sorghum bicolor | 646 | 42.7 | 1.39E−20 |

TABLE 67
Percent identity to Ceres cDNA ID 23517564 (SEQ ID NO: 648)

| | | SEQ ID | % | |
|---|---|---|---|---|
| Designation | Species | NO: | Identity | e-value |
| Ceres CLONE ID no. 936276 | Triticum | 649 | 42.8 | 3.20E−09 |
| | aestivum | | | |
| Ceres CLONE ID no. 234834 | Zea mays | 650 | 49.3 | 1.00E−12 |

TABLE 68
Percent identity to Ceres cDNA ID 23502669 (SEQ ID NO: 652)

| | | SEQ ID | % | |
|---|---|---|---|---|
| Designation | Species | NO: | Identity | e-value |
| Public GI no. 20502805 | Zea mays | 653 | 44.8 | 2.80E−119 |
| Public GI no. 34912988 | Oryza sativa subsp. | 654 | 42.4 | 2.80E−119 |
| | japonica | | | |
| Public GI no. 20467991 | Triticum monococcum | 655 | 41.5 | 6.80E−114 |

TABLE 69
Percent identity to Ceres cDNA ID 23515246 (SEQ ID NO: 659)

| | | SEQ ID | % | |
|---|---|---|---|---|
| Designation | Species | NO: | Identity | e-value |
| Public GI no. 50911537 | Oryza sativa subsp. | 660 | 45.7 | 1.30E−77 |
| | japonica | | | |
| Public GI no. 50911543 | Oryza sativa subsp. | 661 | 43.2 | 8.99E−67 |
| | japonica | | | |
| Ceres CLONE ID no. 788036 | Triticum aestivum | 662 | 40.1 | 5.19E−71 |

TABLE 70
Percent identity to Ceres cDNA ID 24380616 (SEQ ID NO: 664)

| | | SEQ ID | % | |
|---|---|---|---|---|
| Designation | Species | NO: | Identity | e-value |
| Ceres CLONE ID no. 280261 | Zea mays | 665 | 67.7 | 2.30E−100 |
| Public GI no. 50947859 | Oryza sativa subsp. | 666 | 67.8 | 6.79E−101 |
| | japonica | | | |
| Public GI no. 51965036 | Oryza sativa subsp. | 667 | 67.8 | 6.79E−101 |
| | japonica | | | |
| Ceres CLONE ID no. 365048 | Zea mays | 668 | 67.7 | 2.30E−100 |
| Ceres CLONE ID no. 1325022 | Triticum aestivum | 669 | 66.9 | 8.10E−98 |

TABLE 71
Percent identity to Ceres cDNA ID 23503971 (SEQ ID NO: 671)

| | | SEQ ID | % | |
|---|---|---|---|---|
| Designation | Species | NO: | Identity | e-value |
| Public GI no. 28973559 | Arabidopsis | 672 | 90.2 | 0 |
| | thaliana | | | |

TABLE 72
Percent identity to Ceres cDNA ID 23467433 (SEQ ID NO: 674)

| | | SEQ ID | % | |
|---|---|---|---|---|
| Designation | Species | NO: | Identity | e-value |
| Public GI no. 62320769 | Arabidopsis thaliana | 675 | 98.6 | 0 |
| Ceres CLONE ID no. 265352 | Zea mays | 676 | 60.9 | 0 |
| Public GI no. 50928925 | Oryza sativa subsp. | 677 | 59.8 | 0 |
| | japonica | | | |

TABLE 73
Percent identity to Ceres cDNA ID 23554709 (SEQ ID NO: 679)

| | | SEQ ID | % | |
|---|---|---|---|---|
| Designation | Species | NO: | Identity | e-value |
| Public GI no. 30690321 | Arabidopsis thaliana | 680 | 94.4 | 0 |
| Public GI no. 3047075 | Arabidopsis thaliana | 681 | 93.9 | 0 |
| Public GI no. 32402458 | Arabidopsis thaliana | 682 | 93.9 | 0 |
| Public GI no. 32402460 | Arabidopsis thaliana | 683 | 84 | 0 |
| Public GI no. 3047087 | Arabidopsis thaliana | 684 | 83.2 | 0 |

TABLE 74
Percent identity to Ceres cDNA ID 23524514 (SEQ ID NO: 686)

| | | SEQ ID | % | |
|---|---|---|---|---|
| Designation | Species | NO: | Identity | e-value |
| Ceres CLONE ID no. 38286 | Arabidopsis thaliana | 687 | 86.4 | 3.50E−97 |
| Public GI no. 21593352 | Arabidopsis thaliana | 688 | 86.4 | 3.50E−97 |
| Public GI no. 12083200 | Arabidopsis thaliana | 689 | 85.9 | 4.50E−97 |
| Ceres CLONE ID no. 566396 | Glycine max | 690 | 83.2 | 5.39E−85 |
| Public GI no. 5139697 | Cucumis sativus | 691 | 72.8 | 9.99E−66 |
| Ceres CLONE ID no. 1113630 | Glycine max | 692 | 71.8 | 4.49E−65 |
| Public GI no. 53748471 | Plantago major | 693 | 71.6 | 7.99E−75 |

TABLE 75
Percent identity to Ceres cDNA ID 23503210 (SEQ ID NO: 695)

| | | SEQ ID | % | |
|---|---|---|---|---|
| Designation | Species | NO: | Identity | e-value |
| Ceres CLONE ID no. 654820 | Glycine | 696 | 58 | 5.59E−67 |
| | max | | | |

TABLE 76
Percent identity to Ceres cDNA ID 23494809 (SEQ ID NO: 698)

| | | SEQ ID | % | |
|---|---|---|---|---|
| Designation | Species | NO: | Identity | e-value |
| Public GI no. 32455231 | Glycine | 699 | 98.1 | 0 |
| | max | | | |

TABLE 77
Percent identity to Ceres cDNA ID 23740916 (SEQ ID NO: 703)

| | | SEQ ID | % | |
|---|---|---|---|---|
| Designation | Species | NO: | Identity | e-value |
| Public GI no. 20197010 | Arabidopsis thaliana | 704 | 57.4 | 3.39E−67 |
| Ceres CLONE ID no. 114879 | Arabidopsis thaliana | 705 | 57.4 | 4.39E−67 |
| Public GI no. 21536909 | Arabidopsis thaliana | 706 | 57.4 | 4.39E−67 |
| Ceres CLONE ID no. 524672 | Glycine max | 707 | 54.6 | 3.60E−72 |
| Ceres CLONE ID no. 570129 | Triticum aestivum | 708 | 73.6 | 1.39E−111 |
| Public GI no. 53793441 | Oryza sativa subsp. | 709 | 75 | 1.09E−111 |
| | japonica | | | |

TABLE 78
Percent identity to Ceres cDNA ID 23363175 (SEQ ID NO: 711)

| | | SEQ ID | % | |
|---|---|---|---|---|
| Designation | Species | NO: | Identity | e-value |
| Public GI no. 34896098 | Oryza sativa subsp. | 712 | 65.5 | 0 |
| | japonica | | | |
| Ceres CLONE ID no. 930868 | Triticum aestivum | 713 | 37.5 | 2.50E−48 |
| Public GI no. 50949055 | Oryza sativa | 714 | 36.6 | 5.30E−50 |

TABLE 79
Percent identity to Ceres cDNA ID 23421865 (SEQ ID NO: 716)

| | | SEQ ID | % | |
|---|---|---|---|---|
| Designation | Species | NO: | Identity | e-value |
| Public GI no. 27808566 | Arabidopsis thaliana | 717 | 89.4 | 6.39E−121 |
| Ceres CLONE ID no. 710195 | Glycine max | 718 | 68 | 4.59E−79 |
| Ceres CLONE ID no. 222899 | Zea mays | 719 | 59.4 | 2.79E−65 |

TABLE 80
Percent identity to Ceres cDNA ID 23417641 (SEQ ID NO: 721)

| | | SEQ ID | % | |
|---|---|---|---|---|
| Designation | Species | NO: | Identity | e-value |
| Ceres CLONE ID no. 982869 | Brassica napus | 722 | 98.4 | 0 |
| Public GI no. 20258977 | Arabidopsis thaliana | 723 | 84.5 | 1.99E−108 |
| Ceres CLONE ID no. 538662 | Glycine max | 724 | 68.5 | 9.90E−61 |
| Public GI no. 18874263 | Antirrhinum majus | 725 | 67.2 | 3.10E−50 |
| Public GI no. 56605378 | Cucumis sativus | 726 | 67.1 | 8.39E−64 |
| Public GI no. 51557078 | Hevea brasiliensis | 727 | 63.5 | 2.59E−53 |
| Public GI no. 12005328 | Hevea brasiliensis | 728 | 63.5 | 3.29E−53 |
| Ceres CLONE ID no. 833986 | Triticum aestivum | 729 | 60.5 | 2.99E−43 |
| Public GI no. 53749253 | Oryza sativa subsp. | 730 | 58.5 | 1.09E−45 |
| | japonica | | | |

TABLE 81
Percent identity to Ceres cDNA ID 23751471 (SEQ ID NO: 732)

| | | SEQ ID | % | |
|---|---|---|---|---|
| Designation | Species | NO: | Identity | e-value |
| Ceres CLONE ID no. 212540 | Zea mays | 733 | 87.56 | 7.19E−80 |
| Public GI no. 50939031 | Oryza sativa subsp. | 734 | 67.54 | 1.39E−60 |
| | japonica | | | |
| Ceres CLONE ID no. 700212 | Triticum aestivum | 735 | 60.51 | 1.70E−57 |
| Ceres CLONE ID no. 1341109 | Glycine max | 736 | 48.45 | 3.00E−42 |
| Ceres CLONE ID no. 517837 | Glycine max | 737 | 47.96 | 1.90E−40 |
| Public GI no. 16323412 | Arabidopsis thaliana | 738 | 42.64 | 1.49E−31 |
| Public GI no. 21553768 | Arabidopsis thaliana | 739 | 41.62 | 4.00E−31 |
| Ceres CLONE ID no. 16467 | Arabidopsis thaliana | 740 | 41.62 | 4.00E−31 |
| Public GI no. 51970462 | Arabidopsis thaliana | 741 | 38.74 | 4.49E−32 |
| Public GI no. 21592859 | Arabidopsis thaliana | 742 | 38.34 | 3.90E−33 |
| Ceres CLONE ID no. 33347 | Arabidopsis thaliana | 743 | 38.34 | 3.90E−33 |
| Public GI no. 26452180 | Arabidopsis thaliana | 744 | 38.34 | 1.69E−32 |
| Public GI no. 9759459 | Arabidopsis thaliana | 745 | 37.24 | 9.99E−33 |
| Ceres CLONE ID no. 36048 | Arabidopsis thaliana | 746 | 37.24 | 9.99E−33 |

TABLE 82
Percent identity to Ceres cDNA ID 23773450 (SEQ ID NO: 748)

| | | SEQ ID | % | |
|---|---|---|---|---|
| Designation | Species | NO: | Identity | e-value |
| Public GI no. 7446515 | Zea mays | 749 | 98 | 0 |
| Public GI no. 50251892 | Oryza sativa subsp. | 750 | 86.2 | 5.40E−101 |
| | japonica | | | |
| Public GI no. 44888603 | Hordeum vulgare | 751 | 85.2 | 2.49E−103 |
| | subsp. vulgare | | | |
| Public GI no. 3688591 | Triticum aestivum | 752 | 84.8 | 9.70E−102 |
| Public GI no. 13958339 | Poa annua | 753 | 85.2 | 9.70E−102 |
| Public GI no. 28630959 | Lolium perenne | 754 | 84 | 6.79E−101 |
| Public GI no. 40644776 | Triticum aestivum | 755 | 93.6 | 5.90E−63 |
| Public GI no. 47681319 | Dendrocalamus | 756 | 87 | 7.39E−104 |
| | latiflorus | | | |
| Public GI no. 7544096 | Petunia x hybrida | 757 | 68.6 | 1.50E−73 |
| Public GI no. 20385586 | Vitis vinifera | 758 | 73.1 | 5.10E−80 |

TABLE 83
Percent identity to Ceres cDNA ID 23760303 (SEQ ID NO: 760)

| | | SEQ ID | % | |
|---|---|---|---|---|
| Designation | Species | NO: | Identity | e-value |
| Public GI no. 50947859 | Oryza sativa subsp. | 761 | 91.3 | 0 |
| | japonica | | | |
| Public GI no. 51965036 | Oryza sativa subsp. | 762 | 91.3 | 0 |
| | japonica | | | |
| Ceres CLONE ID no. 1325022 | Triticum aestivum | 763 | 85.8 | 0 |
| Ceres CLONE ID no. 1343742 | Arabidopsis thaliana | 764 | 67.7 | 2.30E−100 |

TABLE 84
Percent identity to Ceres cDNA ID 23772039 (SEQ ID NO: 766)

| | | SEQ ID | % | |
|---|---|---|---|---|
| Designation | Species | NO: | Identity | e-value |
| Ceres CLONE ID no. 864432 | Triticum aestivum | 767 | 79.26 | 9.49E−110 |

TABLE 85
Percent identity to Ceres cDNA ID 23792467 (SEQ ID NO: 769)

| | | SEQ ID | % | |
|---|---|---|---|---|
| Designation | Species | NO: | Identity | e-value |
| Public GI no. 32470645 | Solanum | 770 | 63.1 | 1.20E−41 |
| | bulbocastanum | | | |
| Ceres CLONE ID no. 537360 | Glycine max | 771 | 57.9 | 6.00E−70 |
| Public GI no. 30699418 | Arabidopsis thaliana | 772 | 56.4 | 9.90E−61 |
| Public GI no. 4835766 | Arabidopsis thaliana | 773 | 56.1 | 3.20E−64 |
| Ceres CLONE ID no. 677527 | Triticum aestivum | 774 | 50 | 5.79E−40 |
| Public GI no. 4519671 | Nicotiana tabacum | 775 | 45.6 | 3.60E−31 |

TABLE 86
Percent identity to Ceres cDNA ID 23401404 (SEQ ID NO: 777)

| | | SEQ ID | % | |
|---|---|---|---|---|
| Designation | Species | NO: | Identity | e-value |
| Public GI no. 34910914 | Oryza sativa subsp. | 778 | 60.9 | 2.69E−19 |
| | japonica | | | |
| Ceres CLONE ID no. 1064154 | Zea mays | 779 | 58 | 1.40E−38 |
| Ceres CLONE ID no. 113582 | Arabidopsis thaliana | 780 | 55.3 | 2.79E−40 |
| Public GI no. 21536857 | Arabidopsis thaliana | 781 | 55.3 | 2.79E−40 |
| Public GI no. 2894109 | Solanum tuberosum | 782 | 44.2 | 2.69E−19 |
| Ceres CLONE ID no. 686294 | Triticum aestivum | 783 | 43 | 5.20E−16 |
| Public GI no. 436424 | Pisum sativum | 784 | 42.3 | 4.39E−19 |
| Public GI no. 950053 | Hordeum vulgare | 785 | 41.3 | 1.19E−16 |
| | subsp. vulgare | | | |
| Public GI no. 7446213 | Nicotiana tabacum | 786 | 40.9 | 1.99E−16 |
| Public GI no. 729737 | Vicia faba | 787 | 40.8 | 6.90E−21 |
| Public GI no. 7446231 | Canavalia gladiata | 788 | 38.9 | 9.40E−17 |
| Public GI no. 729736 | Ipomoea nil | 789 | 38.3 | 2.40E−18 |
| Public GI no. 1052956 | Ipomoea nil | 790 | 37.5 | 3.90E−18 |

TABLE 87
Percent identity to Ceres cDNA ID 23365746 (SEQ ID NO: 792)

| | | SEQ ID | % | |
|---|---|---|---|---|
| Designation | Species | NO: | Identity | e-value |
| Public GI no. 34907424 | Oryza sativa subsp. | 793 | 60 | 0 |
| | japonica | | | |
| Ceres CLONE ID no. 475016 | Glycine max | 794 | 51.9 | 1.20E−114 |
| Ceres CLONE ID no. 1571937 | Zea mays | 795 | 49.7 | 6.40E−105 |

TABLE 88
Percent identity to Ceres cDNA ID 23765347 (SEQ ID NO: 797)

| | | SEQ ID | % | |
|---|---|---|---|---|
| Designation | Species | NO: | Identity | e-value |
| Public GI no. 50944571 | Oryza sativa subsp. | 798 | 76.7 | 2.29E−86 |
| | japonica | | | |
| Ceres CLONE ID no. 239069 | Zea mays | 799 | 76.6 | 2.20E−79 |
| Ceres CLONE ID no. 677527 | Triticum aestivum | 800 | 71.8 | 5.70E−81 |
| Ceres CLONE ID no. 317477 | Zea mays | 801 | 71.2 | 3.99E−80 |
| Ceres CLONE ID no. 242603 | Zea mays | 802 | 70.8 | 1.99E−78 |
| Ceres CLONE ID no. 38327 | Arabidopsis thaliana | 803 | 66.4 | 1.29E−49 |
| Public GI no. 21593358 | Arabidopsis thaliana | 804 | 66.4 | 1.29E−49 |
| Ceres CLONE ID no. 463968 | Glycine max | 805 | 62.5 | 3.19E−48 |
| Ceres CLONE ID no. 6626 | Arabidopsis thaliana | 806 | 57.7 | 1.00E−51 |
| Public GI no. 21594046 | Arabidopsis thaliana | 807 | 57.7 | 1.00E−51 |
| Public GI no. 42572521 | Arabidopsis thaliana | 808 | 57.7 | 2.40E−50 |
| Ceres CLONE ID no. 581430 | Glycine max | 809 | 57.1 | 7.29E−49 |
| Public GI no. 32470645 | Solanum | 810 | 53.4 | 3.89E−43 |
| | bulbocastanum | | | |

TABLE 89
Percent identity to Ceres cDNA ID 23768927 (SEQ ID NO: 812)

| | | SEQ ID | % | |
|---|---|---|---|---|
| Designation | Species | NO: | Identity | e-value |
| Public GI no. 51964894 | Oryza sativa subsp. | 813 | 71.36 | 5.39E−68 |
| | japonica | | | |
| Public GI no. 16974539 | Arabidopsis thaliana | 814 | 50.5 | 3.50E−41 |
| Ceres CLONE ID no. 557659 | Glycine max | 815 | 41.79 | 8.10E−33 |
| Public GI no. 51964894_T | Artificial Sequence | 816 | 70.5 | 2.00E−76 |
| Public GI no. 16974539_T | Artificial Sequence | 817 | 50.8 | 4.00E−48 |
| Ceres CLONE ID no. 557659_T | Artificial Sequence | 818 | 42.6 | 2.00E−38 |

TABLE 90
Percent identity to Ceres cDNA ID 23495742 (SEQ ID NO: 822)

| | | SEQ ID | % | |
|---|---|---|---|---|
| Designation | Species | NO: | Identity | e-value |
| Public GI no. 57999638 | Closterium | 823 | 54.9 | 7.19E−19 |
| | peracerosum- | | | |
| | strigosum- | | | |
| | littorale | | | |
| | complex | | | |
| Ceres CLONE ID no. 1067477 | Brassica napus | 824 | 51.4 | 3.30E−17 |
| Public GI no. 42795299 | Mimulus lewisii | 825 | 51 | 3.89E−08 |
| Ceres CLONE ID no. 244495 | Zea mays | 826 | 43.6 | 7.90E−20 |

TABLE 91
Percent identity to Ceres cDNA ID 23523867 (SEQ ID NO: 828)

| | | SEQ ID | % | |
|---|---|---|---|---|
| Designation | Species | NO: | Identity | e-value |
| Ceres CLONE ID no. 955910 | Brassica napus | 829 | 55.98 | 1.60E−68 |
| Public GI no. 50939215 | Oryza sativa subsp. | 830 | 21.63 | 2.39E−17 |
| | japonica | | | |
| Public GI no. 50939195 | Oryza sativa | 831 | 24.46 | 7.29E−16 |
| Ceres CLONE ID no. 333937 | Zea mays | 832 | 20.97 | 7.20E−14 |

TABLE 92
Percent identity to Ceres cDNA ID 23516633 (SEQ ID NO: 834)

| | | SEQ ID | % | |
|---|---|---|---|---|
| Designation | Species | NO: | Identity | e-value |
| Public | Arabidopsis thaliana | 835 | 85.3 | 1.09E−70 |
| GI no. 6899920 | | | | |
| Public | Populus tremula x | 836 | 55.2 | 9.80E−38 |
| GI no. 20269055 | Populus tremuloides | | | |
| Public | Populus tremula x | 837 | 53.5 | 1.30E−33 |
| GI no. 20269053 | Populus tremuloides | | | |
| Ceres CLONE | Glycine max | 838 | 50.2 | 6.40E−34 |
| ID no. 675127 | | | | |

TABLE 93
Percent identity to Ceres cDNA ID 23505323 (SEQ ID NO: 840)

| | | SEQ ID | % | |
|---|---|---|---|---|
| Designation | Species | NO: | Identity | e-value |
| Ceres CLONE | Arabidopsis thaliana | 841 | 98.8 | 0 |
| ID no. 15350 | | | | |
| Ceres CLONE | Zea mays | 842 | 61.2 | 3.50E−104 |
| ID no. 300033 | | | | |
| Ceres CLONE | Triticum aestivum | 843 | 51.7 | 2.40E−114 |
| ID no. 557223 | | | | |

TABLE 94
Percent identity to Ceres cDNA ID 23492765 (SEQ ID NO: 845)

| | | SEQ ID | | |
|---|---|---|---|---|
| Designation | Species | NO: | % Identity | e-value |
| Ceres CLONE ID | Glycine max | 846 | 59.6 | 5.00E−72 |
| no. 669185 | | | | |
| Ceres CLONE ID | Zea mays | 847 | 55.8 | 1.20E−53 |
| no. 381106 | | | | |
| Public GI | Oryza sativa subsp. | 848 | 55.3 | 1.80E−54 |
| no. 55297106 | japonica | | | |
| Public GI | Oryza sativa subsp. | 849 | 55 | 3.10E−50 |
| no. 34911652 | japonica | | | |

TABLE 95
Percent identity to Ceres cDNA ID 23486285 (SEQ ID NO: 851)

| | | SEQ ID | % | |
|---|---|---|---|---|
| Designation | Species | NO: | Identity | e-value |
| Ceres CLONE ID | Arabidopsis thaliana | 852 | 94.7 | 0 |
| no. 100484 | | | | |
| Ceres CLONE ID | Triticum aestivum | 853 | 58.3 | 4.00E−96 |
| no. 847458 | | | | |
| Public GI no. | Oryza sativa subsp. | 854 | 58.1 | 9.30E−97 |
| 50909371 | japonica | | | |

TABLE 96
Percent identity to Ceres cDNA ID 23499964 (SEQ ID NO: 856)

| | | SEQ ID | % | |
|---|---|---|---|---|
| Designation | Species | NO: | Identity | e-value |
| Ceres CLONE ID | Glycine max | 857 | 54.8 | 8.00E−43 |
| no. 546084 | | | | |
| Ceres CLONE ID | Zea mays | 858 | 53.6 | 4.39E−42 |
| no. 1567551 | | | | |
| Public GI | Oryza sativa subsp. | 859 | 53.2 | 1.49E−43 |
| no. 50428739 | japonica | | | |
| Ceres CLONE ID | Glycine max | 860 | 52.3 | 3.79E−13 |
| no. 1170120 | | | | |
| Ceres CLONE ID | Zea mays | 861 | 49.7 | 1.00E−42 |
| no. 1603581 | | | | |
| Ceres CLONE ID | Glycine max | 862 | 48 | 4.89E−43 |
| no. 536343 | | | | |
| Ceres CLONE ID | Glycine max | 863 | 44.4 | 1.10E−36 |
| no. 526354 | | | | |
| Ceres CLONE ID | Glycine max | 864 | 43 | 5.79E−40 |
| no. 478622 | | | | |
| Ceres CLONE ID | Glycine max | 865 | 42.6 | 4.19E−37 |
| no. 472335 | | | | |
| Ceres CLONE ID | Triticum aestivum | 866 | 38.8 | 1.30E−33 |
| no. 576107 | | | | |
| Ceres CLONE ID | Zea mays | 867 | 37.1 | 2.00E−23 |
| no. 1503655 | | | | |

TABLE 97
Percent identity to Ceres cDNA ID 4950532 (SEQ ID NO: 871)

| | | SEQ ID | % | |
|---|---|---|---|---|
| Designation | Species | NO: | Identity | e-value |
| Public | Arabidopsis thaliana | 872 | 98.8 | 1.79E−68 |
| GI no. 28394029 | | | | |

TABLE 98
Percent identity to Ceres cDNA ID 23397999 (SEQ ID NO: 874)

| | | SEQ ID | | |
|---|---|---|---|---|
| Designation | Species | NO: | % Identity | e-value |
| Ceres CLONE ID no. 374770 | Zea mays | 875 | 67.1 | 2.00E−50 |
| Public GI no. 21717332 | Malus x domestica | 876 | 64.8 | 4.20E−56 |
| Public GI no. 11181612 | Picea abies | 877 | 64.2 | 1.10E−53 |
| Public GI no. 28894445 | Antirrhinum majus | 878 | 59.5 | 9.79E−54 |
| Public GI no. 20259679 | Arabidopsis thaliana | 879 | 51.4 | 2.90E−59 |
| Public GI no. 42570959 | Arabidopsis thaliana | 880 | 51.4 | 2.20E−56 |
| Public GI no. 25354653 | Arabidopsis thaliana | 881 | 51.4 | 6.79E−56 |
| Public GI no. 34900512 | Oryza sativa subsp. | 882 | 50.4 | 1.59E−54 |
| | japonica | | | |
| Public GI no. 13173164 | Pisum sativum | 883 | 49.3 | 4.59E−56 |
| Public GI no. 51100730 | Ipomoea nil | 884 | 48.1 | 8.70E−58 |
| Public GI no. 5081557 | Petunia x hybrida | 885 | 46.9 | 7.40E−56 |
| Public GI no. 53801434 | Triticum monococcum | 886 | 46.3 | 1.20E−50 |
| Public GI no. 53830031 | Triticum aestivum | 887 | 46.3 | 1.89E−50 |
| | subsp. macha | | | |

TABLE 99
Percent identity to Ceres cDNA ID 23556617 (SEQ ID NO: 889)

| | | SEQ ID | % | |
|---|---|---|---|---|
| Designation | Species | NO: | Identity | e-value |
| Public GI no. 23194453 | Gossypium hirsutum | 890 | 80.1 | 1.79E−86 |
| Public GI no. 60100358 | Lotus japonicus | 891 | 79.2 | 6.89E−85 |
| Public GI no. 3646326 | Malus x domestica | 892 | 78.1 | 1.89E−73 |
| Ceres CLONE ID no. 1044034 | Glycine max | 893 | 77.9 | 4.79E−84 |
| Public GI no. 4103342 | Cucumis sativus | 894 | 77.6 | 2.99E−84 |
| Public GI no. 2997615 | Cucumis sativus | 895 | 77.6 | 1.19E−82 |
| Public GI no. 20385590 | Vitis vinifera | 896 | 77.5 | 5.50E−83 |
| Public GI no. 27763670 | Momordica charantia | 897 | 76.6 | 1.90E−82 |
| Public GI no. 57157565 | Asparagus virgatus | 898 | 70.2 | 8.20E−73 |
| Public GI no. 42794560 | Meliosma dilleniifolia | 899 | 69.9 | 2.50E−71 |
| Public GI no. 29467048 | Agapanthus praecox | 900 | 69.6 | 2.70E−74 |
| Public GI no. 48727598 | Akebia trifoliata | 901 | 68 | 6.49E−73 |
| Public GI no. 21955182 | Hyacinthus orientalis | 902 | 67.5 | 1.39E−70 |
| Public GI no. 1568513 | Petunia x hybrida | 903 | 67.2 | 2.20E−72 |
| Public GI no. 1067169 | Petunia x hybrida | 904 | 66.3 | 2.89E−70 |

TABLE 100
Percent identity to Ceres cDNA ID 23557650 (SEQ ID NO: 906)

| | | SEQ ID | | |
|---|---|---|---|---|
| Designation | Species | NO: | % Identity | e-value |
| Ceres CLONE ID no. 1033993 | Brassica napus | 907 | 95.6 | 5.79E−40 |
| Ceres CLONE ID no. 703180 | Triticum aestivum | 908 | 75.5 | 3.79E−29 |
| Ceres CLONE ID no. 560681 | Glycine max | 909 | 75.5 | 3.79E−29 |
| Ceres CLONE ID no. 562428 | Glycine max | 910 | 75.5 | 3.79E−29 |
| Ceres CLONE ID no. 560948 | Glycine max | 911 | 71.1 | 8.10E−27 |
| Ceres CLONE ID no. 630731 | Glycine max | 912 | 70 | 4.39E−26 |
| Ceres CLONE ID no. 653656 | Glycine max | 913 | 68.5 | 1.60E−23 |
| Ceres CLONE ID no. 663844 | Glycine max | 914 | 67.4 | 4.09E−23 |
| Public GI no. 50929085 | Oryza sativa subsp. | 915 | 59.7 | 1.70E−17 |
| | indica | | | |
| Public GI no. 50912765 | Oryza sativa subsp. | 916 | 52.9 | 1.19E−16 |
| | japonica | | | |
| Ceres CLONE ID no. 503296 | Zea mays | 917 | 50.5 | 7.39E−17 |
| Ceres CLONE ID no. 486120 | Zea mays | 918 | 48.8 | 6.39E−18 |
| Ceres CLONE ID no. 237390 | Zea mays | 919 | 48.2 | 7.30E−10 |

TABLE 101
Percent identity to Ceres cDNA ID 23385560 (SEQ ID NO: 921)

| | | SEQ ID | % | |
|---|---|---|---|---|
| Designation | Species | NO: | Identity | e-value |
| Ceres CLONE | Arabidopsis thaliana | 922 | 81.6 | 2.99E−107 |
| ID no. 1014844 | | | | |
| Public GI no. | Arabidopsis thaliana | 923 | 81.6 | 2.99E−107 |
| 18857720 | | | | |
| Public GI no. | Glycine max | 924 | 72.9 | 8.20E−73 |
| 1234900 | | | | |
| Ceres CLONE | Glycine max | 925 | 71.3 | 2.20E−72 |
| ID no. 527278 | | | | |
| Public GI no. | Pimpinella | 926 | 71 | 8.49E−71 |
| 1149535 | brachycarpa | | | |
| Ceres CLONE | Glycine max | 927 | 68.1 | 8.90E−76 |
| ID no. 514259 | | | | |
| Public GI no. | Capsella rubella | 928 | 61.8 | 1.20E−69 |
| 8919876 | | | | |
| Public GI no. | Lycopersicon | 929 | 61.5 | 8.89E−60 |
| 992598 | esculentum | | | |

TABLE 102
Percent identity to Ceres cDNA ID 23389966 (SEQ ID NO: 931)

| | | SEQ ID | % | |
|---|---|---|---|---|
| Designation | Species | NO: | Identity | e-value |
| Public GI no. 20197615 | Arabidopsis thaliana | 932 | 98.3 | 5.90E−63 |
| Ceres CLONE ID no. 18215 | Arabidopsis thaliana | 933 | 97.5 | 4.00E−64 |
| Public GI no. 21536606 | Arabidopsis thaliana | 934 | 97.5 | 1.19E−62 |
| Ceres CLONE ID no. 105261 | Arabidopsis thaliana | 935 | 97.5 | 1.19E−62 |
| Ceres CLONE ID no. 23214 | Arabidopsis thaliana | 936 | 97.5 | 4.00E−64 |
| Ceres CLONE ID no. 207629 | Arabidopsis thaliana | 937 | 97.2 | 1.40E−54 |
| Ceres CLONE ID no. 24667 | Arabidopsis thaliana | 938 | 96.5 | 1.30E−56 |
| Ceres CLONE ID no. 1006473 | Arabidopsis thaliana | 939 | 96.2 | 3.70E−54 |
| Ceres CLONE ID no. 118878 | Arabidopsis thaliana | 940 | 96.1 | 7.69E−54 |
| Ceres CLONE ID no. 12459 | Arabidopsis thaliana | 941 | 94.1 | 3.50E−58 |
| Ceres CLONE ID no. 1354021 | Arabidopsis thaliana | 942 | 93.3 | 1.50E−57 |
| Public GI no. 30017217 | Arabidopsis thaliana | 943 | 93.3 | 1.50E−57 |
| Ceres CLONE ID no. 109026 | Arabidopsis thaliana | 944 | 91.4 | 1.80E−54 |

TABLE 103
Percent identity to Ceres cDNA ID 23766279 (SEQ ID NO: 946)

| | | SEQ ID | % | |
|---|---|---|---|---|
| Designation | Species | NO: | Identity | e-value |
| Public GI no. 57283093 | Oryza sativa subsp. | 947 | 84.2 | 1.69E−90 |
| | japonica | | | |
| Public GI no. 33621119 | Oryza sativa subsp. | 948 | 84 | 5.60E−90 |
| | japonica | | | |
| Public GI no. 33621117 | Oryza sativa subsp. | 949 | 83.8 | 7.39E−88 |
| | japonica | | | |
| Public GI no. 9367232 | Hordeum vulgare | 950 | 77.8 | 1.49E−82 |
| | subsp. vulgare | | | |
| Public GI no. 9367234 | Hordeum vulgare | 951 | 77.6 | 4.50E−81 |
| | subsp. vulgare | | | |
| Ceres CLONE ID no. 354084 | Zea mays | 952 | 58.6 | 1.90E−57 |
| Public GI no. 29372750 | Zea mays | 953 | 58.6 | 1.90E−57 |
| Public GI no. 10944320 | Arabidopsis thaliana | 954 | 57.3 | 3.60E−56 |
| Public GI no. 51968624 | Arabidopsis thaliana | 955 | 57.3 | 9.50E−56 |
| Public GI no. 33943515 | Brassica rapa | 956 | 56.9 | 3.19E−55 |
| Public GI no. 33943513 | Brassica rapa | 957 | 56.9 | 5.20E−55 |
| Public GI no. 6652756 | Paulownia kawakamii | 958 | 55.9 | 1.80E−54 |
| Public GI no. 16549058 | Magnolia | 959 | 54.6 | 8.50E−55 |
| | praecocissima | | | |
| Public GI no. 30983948 | Eucalyptus occidentalis | 960 | 54.5 | 2.30E−54 |
| Public GI no. 30575602 | Eucalyptus grandis | 961 | 54 | 9.79E−54 |
| Public GI no. 22779230 | Ipomoea batatas | 962 | 52.2 | 1.40E−54 |

TABLE 104
Percent identity to Ceres cDNA ID 23746932 (SEQ ID NO: 964)

| | | SEQ ID | % | |
|---|---|---|---|---|
| Designation | Species | NO: | Identity | e-value |
| Public GI no. | Zea mays | 965 | 94.7 | 5.39E−108 |
| 29372750 | | | | |
| Public GI no. | Oryza sativa subsp. | 966 | 89.8 | 5.50E−99 |
| 62148942 | japonica | | | |
| Public GI no. | Oryza sativa subsp. | 967 | 82.8 | 4.89E−91 |
| 51091146 | japonica | | | |
| Ceres CLONE | Zea mays | 968 | 73 | 5.29E−62 |
| ID no. 300498 | | | | |
| Public GI no. | Zea mays | 969 | 71.7 | 2.20E−79 |
| 29372754 | | | | |
| Ceres CLONE ID | Zea mays | 970 | 71.3 | 5.89E−79 |
| no. 277135 | | | | |
| Public GI no. | Hordeum vulgare | 971 | 62 | 7.00E−60 |
| 9367234 | subsp. vulgare | | | |

TABLE 105
Percent identity to Ceres cDNA ID 23380615 (SEQ ID NO: 973)

| | | SEQ ID | % | |
|---|---|---|---|---|
| Designation | Species | NO: | Identity | e-value |
| Ceres CLONE ID | Arabidopsis thaliana | 974 | 98.8 | 0 |
| no. 7559 | | | | |
| Public GI no. | Zea mays | 975 | 75.2 | 9.59E−79 |
| 52140010 | | | | |
| Ceres CLONE ID | Triticum aestivum | 976 | 72.8 | 2.10E−83 |
| no. 844350 | | | | |
| Public GI no. | Zea mays | 977 | 71.8 | 2.29E−86 |
| 52140009 | | | | |
| Ceres CLONE ID | Zea mays | 978 | 71.8 | 3.69E−86 |
| no. 298172 | | | | |
| Public GI no. | Zea mays | 979 | 70 | 7.50E−79 |
| 52140013 | | | | |
| Ceres CLONE ID | Glycine max | 980 | 68.7 | 7.89E−84 |
| no. 541062 | | | | |
| Public GI no. | Zea mays | 981 | 67.6 | 2.20E−79 |
| 52140015 | | | | |

TABLE 106
Percent identity to Ceres cDNA ID 23366147 (SEQ ID NO: 983)

| | | SEQ ID | % | |
|---|---|---|---|---|
| Designation | Species | NO: | Identity | e-value |
| Ceres CLONE ID no. | Glycine max | 984 | 81.8 | 7.10E−51 |
| 608818 | | | | |
| Ceres CLONE ID no. | Zea mays | 985 | 80.4 | 5.00E−50 |
| 1559765 | | | | |
| Public GI no. 115840 | Zea mays | 986 | 80.4 | 5.00E−50 |
| Public GI no. 22380 | Zea mays | 987 | 80.4 | 5.00E−50 |
| Ceres CLONE ID no. | Zea mays | 988 | 80.3 | 2.40E−50 |
| 1561235 | | | | |
| Ceres CLONE ID no. | Glycine max | 989 | 79.4 | 1.40E−52 |
| 541648 | | | | |
| Ceres CLONE ID no. | Glycine max | 990 | 78.6 | 1.59E−51 |
| 638098 | | | | |

TABLE 107
Percent identity to Ceres cDNA ID 23416775 (SEQ ID NO: 992)

| | | SEQ ID | % | |
|---|---|---|---|---|
| Designation | Species | NO: | Identity | e-value |
| Ceres CLONE ID no. 1091297 | Brassica napus | 993 | 72.6 | 2.90E−29 |
| Public GI no. 33324520 | Gossypium hirsutum | 994 | 72 | 3.29E−37 |
| Public GI no. 55741382 | Oryza sativa subsp. | 995 | 65 | 5.29E−30 |
| | japonica | | | |
| Ceres CLONE ID no. 471446 | Glycine max | 996 | 64 | 7.89E−36 |
| Ceres CLONE ID no. 472054 | Glycine max | 997 | 63.5 | 3.00E−36 |
| Ceres CLONE ID no. 1050656 | Glycine max | 998 | 63.2 | 8.80E−37 |
| Public GI no. 31324058 | Glycine max | 999 | 63.2 | 8.80E−37 |

TABLE 108
Percent identity to Ceres cDNA ID 23359888 (SEQ ID NO: 1001)

| | | SEQ ID | % | |
|---|---|---|---|---|
| Designation | Species | NO: | Identity | e-value |
| Ceres CLONE ID no. 30700 | Arabidopsis thaliana | 1002 | 93.8 | 0 |
| Public GI no. 23397033 | Arabidopsis thaliana | 1003 | 93.8 | 0 |
| Public GI no. 19698881 | Arabidopsis thaliana | 1004 | 93.5 | 0 |
| Public GI no. 19697 | Nicotiana | 1005 | 93.5 | 0 |
| | plumbaginifolia | | | |
| Public GI no. 21555870 | Arabidopsis thaliana | 1006 | 93.5 | 0 |
| Public GI no. 475216 | Nicotiana tabacum | 1007 | 93.2 | 0 |
| Public GI no. 2119938 | Nicotiana tabacum | 1008 | 93.2 | 0 |
| Public GI no. 2119934 | Nicotiana tabacum | 1009 | 93.2 | 0 |
| Public GI no. 2119932 | Nicotiana tabacum | 1010 | 93 | 0 |
| Public GI no. 485949 | Nicotiana tabacum | 1011 | 93 | 0 |
| Public GI no. 485945 | Nicotiana tabacum | 1012 | 93 | 0 |
| Public GI no. 485943 | Nicotiana tabacum | 1013 | 93 | 0 |
| Public GI no. 2119933 | Nicotiana tabacum | 1014 | 93 | 0 |
| Public GI no. 485951 | Nicotiana tabacum | 1015 | 92.7 | 0 |
| Public GI no. 485987 | Nicotiana tabacum | 1016 | 92.7 | 0 |
| Public GI no. 25809054 | Pisum sativum | 1017 | 92.4 | 0 |

TABLE 109
Percent identity to Ceres cDNA ID 23385230 (SEQ ID NO: 1019)

| | | SEQ ID | % | |
|---|---|---|---|---|
| Designation | Species | NO: | Identity | e-value |
| Public GI no. 25405956 | Arabidopsis thaliana | 1020 | 84 | 1.49E−14 |
| Public GI no. 30694486 | Arabidopsis thaliana | 1021 | 84 | 2.40E−14 |
| Ceres CLONE ID no. 354956 | Zea mays | 1022 | 69.7 | 1.49E−10 |
| Public GI no. 22854970 | Brassica nigra | 1023 | 60 | 4.89E−08 |
| Public GI no. 22854950 | Brassica nigra | 1024 | 60 | 4.89E−08 |

TABLE 110
Percent identity to Ceres cDNA ID 23359443 (SEQ ID NO: 1026)

| | | SEQ ID | % | |
|---|---|---|---|---|
| Designation | Species | NO: | Identity | e-value |
| Public GI no. 1806261 | Petroselinum crispum | 1027 | 48.6 | 3.00E−52 |
| Public GI no. 100163 | Petroselinum crispum | 1028 | 48.3 | 7.29E−49 |
| Public GI no. 542187 | Zea mays | 1029 | 43.7 | 1.70E−42 |
| Public GI no. 168428 | Zea mays | 1030 | 43.6 | 8.90E−44 |
| Public GI no. 15865782 | Oryza sativa subsp. | 1031 | 43 | 1.40E−40 |
| | indica | | | |
| Ceres CLONE ID no. 235570 | Zea mays | 1032 | 42.9 | 4.89E−43 |
| Public GI no. 16797791 | Nicotiana tabacum | 1033 | 42.9 | 5.40E−53 |
| Ceres CLONE ID no. 298319 | Zea mays | 1034 | 42.8 | 1.49E−43 |
| Ceres CLONE ID no. 295738 | Zea mays | 1035 | 42.7 | 4.89E−43 |
| Public GI no. 34897226 | Oryza sativa subsp. | 1036 | 42.2 | 1.70E−42 |
| | japonica | | | |
| Public GI no. 1869928 | Hordeum vulgare | 1037 | 42.1 | 3.59E−40 |
| | subsp. vulgare | | | |
| Public GI no. 1144536 | Zea mays | 1038 | 41.8 | 1.89E−41 |
| Public GI no. 4115746 | Oryza sativa subsp. | 1039 | 41.5 | 5.70E−42 |
| | indica | | | |
| Public GI no. 7489532 | Oryza sativa subsp. | 1040 | 41.4 | 1.00E−42 |
| | Indica | | | |

TABLE 111
Percent identity to Ceres cDNA ID 23386664 (SEQ ID NO: 1042)

| | | SEQ ID | % | |
|---|---|---|---|---|
| Designation | Species | NO: | Identity | e-value |
| Public GI no. 14030607 | Arabidopsis thaliana | 1043 | 98.8 | 5.79E−40 |
| Public GI no. 5107082 | Arabidopsis thaliana | 1044 | 97.6 | 5.19E−39 |
| Ceres CLONE ID no. 1090803 | Brassica napus | 1045 | 75 | 6.99E−28 |
| Ceres CLONE ID no. 946808 | Brassica napus | 1046 | 75 | 3.50E−26 |
| Ceres CLONE ID no. 1086365 | Brassica napus | 1047 | 73.8 | 8.99E−28 |
| Ceres CLONE ID no. 1323425 | Triticum aestivum | 1048 | 71 | 2.49E−25 |
| Ceres CLONE ID no. 617980 | Triticum aestivum | 1049 | 68.9 | 1.29E−28 |
| Ceres CLONE ID no. 373100 | Zea mays | 1050 | 68.6 | 1.90E−27 |
| Public GI no. 50251897 | Oryza sativa subsp. | 1051 | 67.9 | 9.50E−24 |
| | japonica | | | |
| Public GI no. 5107149 | Oryza sativa | 1052 | 67.5 | 6.70E−23 |
| Public GI no. 50928231 | Oryza sativa subsp. | 1053 | 65.8 | 3.99E−25 |
| | japonica | | | |
| Ceres CLONE ID no. 714267 | Glycine max | 1054 | 60.7 | 4.69E−22 |
| Ceres CLONE ID no. 584348 | Glycine max | 1055 | 59.3 | 5.99E−22 |
| Public GI no. 5107157 | Malus x domestica | 1056 | 35.4 | 6.69E−07 |

TABLE 112
Percent identity to Ceres cDNA ID 23371818 (SEQ ID NO: 1058)

| | | SEQ ID | % | |
|---|---|---|---|---|
| Designation | Species | NO: | Identity | e-value |
| Public GI no. 15810073 | Arabidopsis thaliana | 1059 | 98.5 | 0 |
| Ceres CLONE ID no. 285163 | Zea mays | 1060 | 61.6 | 4.09E−46 |
| Public GI no. 50906555 | Oryza sativa subsp. | 1061 | 61.2 | 8.49E−46 |
| | japonica | | | |
| Public GI no. 34909384 | Oryza sativa subsp. | 1062 | 54.4 | 7.59E−49 |
| | japonica | | | |
| Public GI no. 17976835 | Pinus pinaster | 1063 | 47.5 | 1.90E−25 |
| Public GI no. 32396295 | Pinus taeda | 1064 | 47.5 | 2.40E−25 |
| Public GI no. 16610193 | Nicotiana tabacum | 1065 | 45.2 | 3.90E−25 |
| Public GI no. 20269057 | Populus tremula x | 1066 | 44.4 | 1.70E−24 |
| | Populus tremuloides | | | |

TABLE 113
Percent identity to Ceres cDNA ID 23471864 (SEQ ID NO: 1068)

| | | SEQ ID | % | |
|---|---|---|---|---|
| Designation | Species | NO: | Identity | e-value |
| Ceres CLONE ID no. 647941 | Glycine max | 1069 | 95.4 | 4.79E−29 |
| Ceres CLONE ID no. 1246527 | Glycine max | 1070 | 91 | 1.29E−28 |
| Ceres CLONE ID no. 1306476 | Brassica napus | 1071 | 86.5 | 1.30E−37 |
| Ceres CLONE ID no. 1259850 | Brassica napus | 1072 | 79.5 | 1.50E−32 |

TABLE 114
Percent identity to Ceres cDNA ID 23370870 (SEQ ID NO: 1074)

| | | SEQ ID | % | |
|---|---|---|---|---|
| Designation | Species | NO: | Identity | e-value |
| Public GI no. 47680447 | Triticum aestivum | 1075 | 69.5 | 1.80E−51 |
| Ceres CLONE ID no. 540373 | Glycine max | 1076 | 67.3 | 5.19E−48 |
| Ceres CLONE ID no. 347485 | Zea mays | 1077 | 65.7 | 3.99E−58 |
| Public GI no. 1370140 | Lycopersicon | 1078 | 63.5 | 4.49E−49 |
| | esculentum | | | |
| Public GI no. 20561 | Petunia x hybrida | 1079 | 62 | 7.29E−49 |
| Public GI no. 32489375 | Oryza sativa subsp. | 1080 | 58.6 | 1.50E−58 |
| | japonica | | | |
| Public GI no. 22266673 | Vitis labrusca x Vitis | 1081 | 54.7 | 2.80E−49 |
| | vinifera | | | |
| Public GI no. 22266675 | Vitis labrusca x Vitis | 1082 | 54.7 | 2.80E−49 |
| | vinifera | | | |
| Public GI no. 1732247 | Nicotiana tabacum | 1083 | 53.7 | 9.40E−49 |
| Public GI no. 5139814 | Glycine max | 1084 | 53.5 | 1.00E−49 |
| Public GI no. 6552361 | Nicotiana tabacum | 1085 | 47.6 | 5.60E−51 |

TABLE 115
Percent identity to Ceres cDNA ID 23361688 (SEQ ID NO: 1087)

| | | SEQ ID | % | |
|---|---|---|---|---|
| Designation | Species | NO: | Identity | e-value |
| Ceres CLONE ID no. 280394 | Zea mays | 1088 | 82.9 | 1.60E−62 |
| Public GI no. 50945939 | Oryza sativa | 1089 | 82.8 | 7.59E−65 |
| Public GI no. 19073336 | Sorghum bicolor | 1090 | 81.6 | 3.39E−60 |
| Public GI no. 19073332 | Sorghum bicolor | 1091 | 81.3 | 3.69E−61 |
| Ceres CLONE ID no. 1061835 | Zea mays | 1092 | 80.5 | 1.39E−61 |
| Public GI no. 19073330 | Sorghum bicolor | 1093 | 80.5 | 2.10E−60 |
| Public GI no. 13346188 | Gossypium hirsutum | 1094 | 70.3 | 1.10E−54 |
| Public GI no. 6651292 | Pimpinella | 1095 | 68.1 | 4.29E−76 |
| | brachycarpa | | | |
| Public GI no. 1430846 | Lycopersicon | 1096 | 67.7 | 1.89E−64 |
| | esculentum | | | |
| Public GI no. 34147926 | Pinus taeda | 1097 | 59.5 | 4.40E−58 |
| Public GI no. 50948253 | Oryza sativa subsp. | 1098 | 58.4 | 1.19E−62 |
| | japonica | | | |
| Public GI no. 50725788 | Oryza sativa subsp. | 1099 | 57.2 | 9.99E−66 |
| | japonica | | | |
| Public GI no. 23343579 | Oryza sativa | 1100 | 56.8 | 1.19E−64 |

TABLE 116
Percent identity to Ceres cDNA ID 23448883 (SEQ ID NO: 1102)

| | | SEQ ID | % | |
|---|---|---|---|---|
| Designation | Species | NO: | Identity | e-value |
| Ceres CLONE ID no. 92459 | Arabidopsis thaliana | 1103 | 93.2 | 9.99E−84 |
| Public GI no. 21617978 | Arabidopsis thaliana | 1104 | 93.2 | 9.99E−84 |
| Public GI no. 2829920 | Arabidopsis thaliana | 1105 | 91.6 | 3.19E−80 |
| Public GI no. 31580813 | Brassica napus | 1106 | 63.6 | 3.40E−51 |
| Ceres CLONE ID no. 1065387 | Brassica napus | 1107 | 63.5 | 4.20E−53 |
| Public GI no. 17933450 | Brassica napus | 1108 | 63.5 | 4.20E−53 |
| Public GI no. 17933458 | Brassica napus | 1109 | 62.8 | 4.40E−51 |
| Ceres CLONE ID no. 1091989 | Brassica napus | 1110 | 62.6 | 7.10E−51 |
| Public GI no. 17933456 | Brassica napus | 1111 | 62.6 | 7.10E−51 |
| Public GI no. 34591565 | Brassica oleracea var. | 1112 | 62.6 | 1.50E−50 |
| | capitata | | | |
| Public GI no. 30523250 | Raphanus sativus | 1113 | 61.7 | 1.00E−49 |
| Public GI no. 30523252 | Brassica oleracea var. | 1114 | 59.3 | 5.00E−50 |
| | capitata | | | |
| Public GI no. 45181459 | Brassica rapa | 1115 | 59.3 | 2.19E−49 |
| Ceres CLONE ID no. 963001 | Brassica napus | 1116 | 59.3 | 2.80E−49 |
| Public GI no. 30523362 | Brassica napus | 1117 | 59.3 | 2.80E−49 |

TABLE 117
Percent identity to Ceres cDNA ID 23389186 (SEQ ID NO: 1119)

| | | SEQ ID | % | |
|---|---|---|---|---|
| Designation | Species | NO: | Identity | e-value |
| Ceres CLONE ID no. 625275 | Glycine max | 1120 | 68.6 | 3.20E−87 |
| Ceres CLONE ID no. 1246429 | Glycine max | 1121 | 65.7 | 1.60E−83 |
| Public GI no. 37718893 | Oryza sativa subsp. | 1122 | 64.2 | 3.50E−81 |
| | japonica | | | |
| Ceres CLONE ID no. 937503 | Triticum aestivum | 1123 | 63.3 | 7.30E−81 |
| Ceres CLONE ID no. 400568 | Zea mays | 1124 | 60.3 | 1.29E−76 |
| Ceres CLONE ID no. 1549251 | Zea mays | 1125 | 60.3 | 5.60E−74 |

TABLE 118
Percent identity to Ceres cDNA ID 23380898 (SEQ ID NO: 1127)

| | | SEQ ID | % | |
|---|---|---|---|---|
| Designation | Species | NO: | Identity | e-value |
| Ceres CLONE ID no. 13879 | Arabidopsis thaliana | 1128 | 98.8 | 3.09E−89 |
| Public GI no. 21553354 | Arabidopsis thaliana | 1129 | 98.8 | 3.09E−89 |
| Ceres CLONE ID no. 158026 | Arabidopsis thaliana | 1130 | 98.6 | 3.40E−76 |
| Ceres CLONE ID no. 1012104 | Arabidopsis thaliana | 1131 | 98.6 | 5.39E−69 |
| Public GI no. 1346180 | Sinapis alba | 1132 | 92.5 | 1.50E−73 |
| Public GI no. 1346181 | Sinapis alba | 1133 | 87.3 | 5.50E−76 |
| Public GI no. 17819 | Brassica napus | 1134 | 81.25 | 4.00E−64 |
| Public GI no. 34851124 | Prunus avium | 1135 | 79.5 | 2.49E−64 |
| Ceres CLONE ID no. 583672 | Glycine max | 1136 | 78.1 | 1.09E−63 |

TABLE 119
Percent identity to Ceres cDNA ID 23383311 (SEQ ID NO: 1138)

| | | SEQ ID | % | |
|---|---|---|---|---|
| Designation | Species | NO: | Identity | e-value |
| Ceres CLONE ID no. 659723 | Glycine max | 1139 | 93.8 | 1.89E−34 |
| Ceres CLONE ID no. 953644 | Brassica napus | 1140 | 87.4 | 1.20E−69 |
| Ceres CLONE ID no. 1585988 | Zea mays | 1141 | 73.4 | 1.20E−39 |
| Ceres CLONE ID no. 245683 | Zea mays | 1142 | 72.4 | 1.79E−29 |
| Ceres CLONE ID no. 1283552 | Zea mays | 1143 | 72.4 | 1.79E−29 |
| Ceres CLONE ID no. 272426 | Zea mays | 1144 | 69.3 | 3.39E−33 |
| Ceres CLONE ID no. 824827 | Triticum aestivum | 1145 | 68.8 | 1.89E−34 |

TABLE 120
Percent identity to Ceres cDNA ID 23384792 (SEQ ID NO: 1147)

| | | SEQ ID | % | |
|---|---|---|---|---|
| Designation | Species | NO: | Identity | e-value |
| Ceres CLONE ID no. 467528 | Glycine max | 1148 | 54.5 | 5.79E−40 |
| Public GI no. 20269057 | Populus tremula x | 1149 | 52 | 2.39E−43 |
| | Populus tremuloides | | | |
| Public GI no. 51964528 | Oryza sativa subsp. | 1150 | 51 | 2.20E−33 |
| | japonica | | | |
| Public GI no. 50915894 | Oryza sativa subsp. | 1151 | 50 | 3.50E−33 |
| | japonica | | | |
| Public GI no. 32396299 | Pinus taeda | 1152 | 49 | 3.80E−23 |
| Public GI no. 62120254 | Oryza sativa subsp. | 1153 | 48.6 | 2.90E−29 |
| | indica | | | |
| Public GI no. 4887020 | Nicotiana tabacum | 1154 | 46.7 | 5.40E−21 |
| Public GI no. 4887022 | Nicotiana tabacum | 1155 | 44.5 | 4.69E−22 |
| Ceres CLONE ID no. 305337 | Zea mays | 1156 | 38.1 | 7.50E−24 |

TABLE 121
Percent identity to Ceres cDNA ID 23360311 (SEQ ID NO: 1158)

| | | SEQ ID | % | |
|---|---|---|---|---|
| Designation | Species | NO: | Identity | e-value |
| Ceres CLONE ID no. 627169 | Glycine max | 1159 | 76.6 | 9.99E−86 |
| Public GI no. 34914598 | Oryza sativa subsp. | 1160 | 73 | 1.99E−78 |
| | japonica | | | |
| Ceres CLONE ID no. 1397168 | Zea mays | 1161 | 70.6 | 1.59E−76 |
| Public GI no. 50909895 | Oryza sativa subsp. | 1162 | 66.9 | 2.09E−67 |
| | japonica | | | |
| Ceres CLONE ID no. 704527 | Triticum aestivum | 1163 | 65.8 | 8.09E−66 |

TABLE 122
Percent identity to Ceres cDNA ID 23375896 (SEQ ID NO: 1165)

| | | SEQ | | |
|---|---|---|---|---|
| | | ID | % | |
| Designation | Species | NO: | Identity | e-value |
| Ceres CLONE ID no. 476024 | Glycine max | 1166 | 71.1 | 1.19E−55 |
| Ceres CLONE ID no. | Triticum | 1167 | 63.8 | 2.10E−42 |
| 1017044 | aestivum | | | |
| Ceres CLONE ID no. 230052 | Zea mays | 1168 | 61.2 | 6.29E−43 |
| Ceres CLONE ID no. 341096 | Zea mays | 1169 | 58.2 | 1.59E−37 |

TABLE 123
Percent identity to Ceres cDNA ID 23376628 (SEQ ID NO: 1171)

| | | SEQ | | |
|---|---|---|---|---|
| | | ID | % | |
| Designation | Species | NO: | Identity | e-value |
| Ceres CLONE ID no. 636599 | Glycine max | 1172 | 63.3 | 7.09E−83 |
| Public GI no. 50934801 | Oryza sativa | 1173 | 53.7 | 1.19E−64 |
| | subsp. | | | |
| | japonica | | | |
| Public GI no. 31712074 | Oryza sativa | 1174 | 52.8 | 1.70E−56 |
| | subsp. | | | |
| | japonica | | | |
| Ceres CLONE ID no. 696154 | Triticum | 1175 | 51.4 | 2.90E−54 |
| | aestivum | | | |
| Ceres CLONE ID no. | Zea mays | 1176 | 50.8 | 7.19E−58 |
| 1554290 | | | | |

TABLE 124
Percent identity to Ceres cDNA ID 23369842 (SEQ ID NO: 1178)

| | | SEQ ID | % | |
|---|---|---|---|---|
| Designation | Species | NO: | Identity | e-value |
| Public GI no. 8809670 | Arabidopsis thaliana | 1179 | 81.3 | 8.00E−107 |
| Ceres CLONE ID no. 254065 | Arabidopsis thaliana | 1180 | 81.1 | 1.79E−107 |
| Public GI no. 38564314 | Arabidopsis thaliana | 1181 | 81.1 | 1.79E−107 |
| Ceres CLONE ID no. 477450 | Glycine max | 1182 | 73.6 | 3.60E−95 |
| Ceres CLONE ID no. 280814 | Zea mays | 1183 | 67.6 | 7.99E−75 |
| Public GI no. 55775124 | Oryza sativa subsp. | 1184 | 67.2 | 4.00E−73 |
| | japonica | | | |
| Ceres CLONE ID no. 295114 | Zea mays | 1185 | 66.6 | 1.80E−77 |
| Ceres CLONE ID no. 241340 | Zea mays | 1186 | 62.7 | 1.70E−79 |
| Public GI no. 32489377 | Oryza sativa subsp. | 1187 | 60.7 | 7.09E−83 |
| | japonica | | | |
| Ceres CLONE ID no. 700178 | Triticum aestivum | 1188 | 59 | 2.20E−79 |
| Public GI no. 50928853 | Oryza sativa subsp. | 1189 | 57.4 | 7.99E−75 |
| | japonica | | | |
| Public GI no. 50918277 | Oryza sativa subsp. | 1190 | 56.25 | 2.59E−69 |
| | japonica | | | |

TABLE 125
Percent identity to Ceres cDNA ID 23416869 (SEQ ID NO: 1192)

| | | SEQ | | |
|---|---|---|---|---|
| | | ID | % | |
| Designation | Species | NO: | Identity | e-value |
| Ceres CLONE ID no. 738705 | Triticum | 1193 | 59.3 | 5.19E−39 |
| | aestivum | | | |
| Ceres CLONE ID no. 892214 | Triticum | 1194 | 59.3 | 1.10E−38 |
| | aestivum | | | |
| Public GI no. 50913251 | Oryza sativa | 1195 | 58 | 4.70E−38 |
| | subsp. | | | |
| | japonica | | | |
| Ceres CLONE ID no. 341749 | Zea mays | 1196 | 55.7 | 2.30E−36 |
| Ceres CLONE ID no. 666962 | Glycine max | 1197 | 55.5 | 1.59E−37 |
| Ceres CLONE ID no. 522672 | Glycine max | 1198 | 55.5 | 3.29E−37 |
| Public GI no. 11602747 | Vicia faba | 1199 | 54 | 4.39E−35 |
| Public GI no. 11602749 | Vicia faba | 1200 | 53.7 | 1.30E−33 |

TABLE 126
Percent identity to Ceres cDNA ID 23785125 (SEQ ID NO: 1202)

| | | SEQ | | |
|---|---|---|---|---|
| | | ID | % | |
| Designation | Species | NO: | Identity | e-value |
| Ceres CLONE ID no. 841321 | Triticum | 1203 | 79.8 | 8.39E−48 |
| | aestivum | | | |
| Public GI no. 55773842 | Oryza sativa | 1204 | 69.6 | 4.79E−61 |
| | subsp. | | | |
| | japonica | | | |
| Ceres CLONE ID no. 601248 | Glycine max | 1205 | 63.6 | 7.20E−42 |
| Public GI no. 42794937 | Arabidopsis | 1206 | 60.3 | 3.99E−41 |
| | thaliana | | | |
| Ceres CLONE ID no. 959875 | Brassica | 1207 | 59.5 | 1.10E−40 |
| | napus | | | |
| Public GI no. 28372932 | Arabidopsis | 1208 | 59.5 | 1.10E−40 |
| | thaliana | | | |

TABLE 127
Percent identity to Ceres cDNA ID 23699071 (SEQ ID NO: 1212)

| | | SEQ ID | % | |
|---|---|---|---|---|
| Designation | Species | NO: | Identity | e-value |
| Ceres CLONE ID no. | Glycine max | 1213 | 79.5 | 6.10E−77 |
| 643026 | | | | |
| Public GI no. 31430853 | Oryza sativa | 1214 | 66.3 | 5.00E−98 |
| | subsp. | | | |
| | japonica | | | |
| Ceres CLONE ID no. | Zea mays | 1215 | 65 | 5.50E−99 |
| 329797 | | | | |
| Ceres CLONE ID no. | Arabidopsis | 1216 | 64.4 | 8.50E−103 |
| 38757 | thaliana | | | |
| Public GI no. 30681003 | Arabidopsis | 1217 | 62.2 | 9.40E−88 |
| | thaliana | | | |
| Ceres CLONE ID no. | Triticum | 1218 | 51.1 | 3.49E−65 |
| 570295 | aestivum | | | |

TABLE 128
Percent identity to Ceres cDNA ID 23527182 (SEQ ID NO: 1220)

| | | SEQ ID | % | |
|---|---|---|---|---|
| Designation | Species | NO: | Identity | e-value |
| Ceres CLONE ID no. | Arabidopsis | 1221 | 82.7 | 1.99E−124 |
| 1334990 | thaliana | | | |
| Public GI no. 20466045 | Arabidopsis | 1222 | 82.7 | 1.99E−124 |
| | thaliana | | | |
| Public GI no. 12711287 | Nicotiana | 1223 | 49.5 | 1.10E−40 |
| | tabacum | | | |
| Ceres CLONE ID no. | Glycine max | 1224 | 41.9 | 1.70E−42 |
| 473814 | | | | |

TABLE 129
Percent identity to Ceres cDNA ID 23747378 (SEQ ID NO: 1226)

| | | SEQ ID | | |
|---|---|---|---|---|
| Designation | Species | NO: | % Identity | e-value |
| Public GI no. 62122347 | Ginkgo biloba | 1227 | 39.8 | 9.99E−27 |
| Public GI no. 5019464 | Gnetum gnemon | 1228 | 39.8 | 3.10E−25 |
| Public GI no. 51849631 | Euryale ferox | 1229 | 40.5 | 6.50E−25 |
| Public GI no. 51849641 | Brasenia schreberi | 1230 | 41.5 | 1.39E−24 |
| Public GI no. 51849637 | Cabomba caroliniana | 1231 | 44.5 | 6.50E−25 |
| Ceres CLONE ID no. 700266 | Triticum aestivum | 1232 | 76.6 | 9.59E−79 |
| Ceres CLONE ID no. 465896 | Zea mays | 1233 | 90.3 | 1.49E−89 |
| Ceres CLONE ID no. 302467 | Zea mays | 1234 | 89.4 | 8.90E−76 |
| Public GI no. 37993053 | Eupomatia bennettii | 1235 | 38.2 | 3.99E−25 |
| Public GI no. 37993051 | Eupomatia bennettii | 1236 | 37.6 | 8.29E−25 |
| Public GI no. 34910770 | Oryza sativa subsp. | 1237 | 80.6 | 9.59E−79 |
| | japonica | | | |
| Public GI no. 51849651 | Nuphar japonica | 1238 | 42.1 | 1.49E−25 |
| Public GI no. 51849649 | Nuphar japonica | 1239 | 41.1 | 2.49E−25 |
| Public GI no. 51849635 | Nymphaea tetragona | 1240 | 41.1 | 2.49E−25 |
| Public GI no. 62867345 | Agapanthus praecox | 1241 | 37.5 | 1.39E−24 |

TABLE 130
Percent identity to Ceres cDNA ID 23691708 (SEQ ID NO: 1243)

| | | SEQ | | |
|---|---|---|---|---|
| | | ID | % | |
| Designation | Species | NO: | Identity | e-value |
| Public GI no. 9755785 | Arabidopsis | 1244 | 56.4 | 5.50E−60 |
| | thaliana | | | |
| Ceres CLONE ID no. 833439 | Triticum | 1245 | 52.8 | 7.10E−51 |
| | aestivum | | | |
| Public GI no. 50911677 | Oryza sativa | 1246 | 51.5 | 1.10E−54 |
| | subsp. | | | |
| | japonica | | | |

TABLE 131
Percent identity to Ceres cDNA ID 23697027 (SEQ ID NO: 1248)

| | | SEQ ID | % | |
|---|---|---|---|---|
| Designation | Species | NO: | Identity | e-value |
| Public GI no. 23197970 | Arabidopsis | 1249 | 79.5 | 0 |
| | thaliana | | | |
| Ceres CLONE ID no. 578919 | Glycine max | 1250 | 62.6 | 0 |
| Public GI no. 34909052 | Oryza sativa | 1251 | 61.4 | 0 |
| | subsp. | | | |
| | japonica | | | |
| Public GI no. 50939567 | Oryza sativa | 1252 | 55.4 | 0 |
| | subsp. | | | |
| | japonica | | | |
| Ceres CLONE ID no. 504165 | Zea mays | 1253 | 54.8 | 0 |

TABLE 132
Percent identity to Ceres cDNA ID 23416843 (SEQ ID NO: 1255)

| | | SEQ | | |
|---|---|---|---|---|
| | | ID | % | |
| Designation | Species | NO: | Identity | e-value |
| Ceres CLONE ID no. 554630 | Glycine max | 1256 | 51.5 | 2.10E−60 |
| Public GI no. 50911677 | Oryza sativa | 1257 | 50.3 | 1.19E−57 |
| | subsp. | | | |
| | japonica | | | |
| Ceres CLONE ID no. 655359 | Glycine max | 1258 | 48.4 | 5.50E−60 |
| Ceres CLONE ID no. 833439 | Triticum | 1259 | 46.1 | 9.79E−54 |
| | aestivum | | | |

TABLE 133
Percent identity to Ceres cDNA ID 23449314 (SEQ ID NO: 1261)

| | | SEQ | | |
|---|---|---|---|---|
| | | ID | % | |
| Designation | Species | NO: | Identity | e-value |
| Public GI no. 56749359 | Arabidopsis thaliana | 1262 | 98.5 | 0 |
| Public GI no. 3941412 | Arabidopsis thaliana | 1263 | 98.2 | 0 |
| Public GI no. 28628965 | Dendrobium sp. XMW- | 1264 | 89.6 | 3.60E−69 |
| | 2002-10 | | | |
| Ceres CLONE ID no. 1560573 | Zea mays | 1265 | 77.9 | 2.80E−71 |
| Public GI no. 82308 | Antirrhinum majus | 1266 | 72.9 | 3.70E−77 |
| Public GI no. 13346194 | Gossypium hirsutum | 1267 | 72.3 | 1.09E−84 |
| Public GI no. 42541167 | Tradescantia | 1268 | 69.5 | 2.99E−68 |
| | fluminensis | | | |
| Public GI no. 39725415 | Eucalyptus gunnii | 1269 | 68.4 | 1.40E−75 |
| Public GI no. 31980095 | Populus tremula x | 1270 | 67.8 | 1.30E−81 |
| | Populus tremuloides | | | |
| Public GI no. 1167484 | Lycopersicon | 1271 | 66.7 | 5.50E−83 |
| | esculentum | | | |
| Public GI no. 50726662 | Oryza sativa subsp. | 1272 | 66 | 3.50E−74 |
| | japonica | | | |
| Public GI no. 19053 | Hordeum vulgare | 1273 | 65.6 | 5.39E−69 |
| | subsp. vulgare | | | |
| Public GI no. 19072766 | Oryza sativa subsp. | 1274 | 65 | 2.09E−67 |
| | japonica | | | |
| Public GI no. 50948275 | Oryza sativa subsp. | 1275 | 64.6 | 1.20E−69 |
| | japonica | | | |
| Ceres CLONE ID no. 1459729 | Zea mays | 1276 | 63.8 | 4.00E−73 |
| Public GI no. 47680445 | Triticum aestivum | 1277 | 63 | 4.10E−72 |

TABLE 134
Percent identity to Ceres cDNA ID 23390282 (SEQ ID NO: 1279)

| | | SEQ ID | % | |
|---|---|---|---|---|
| Designation | Species | NO: | Identity | e-value |
| Ceres CLONE ID no. 3244 | Arabidopsis thaliana | 1280 | 98 | 1.00E−51 |
| Ceres CLONE ID no. 12459 | Arabidopsis thaliana | 1281 | 98 | 1.09E−47 |
| Ceres CLONE ID no. 39985 | Arabidopsis thaliana | 1282 | 97 | 3.80E−52 |
| Ceres CLONE ID no. 1354021 | Arabidopsis thaliana | 1283 | 97 | 4.60E−47 |
| Public GI no. 30017217 | Arabidopsis thaliana | 1284 | 97 | 4.60E−47 |
| Ceres CLONE ID no. 114551 | Arabidopsis thaliana | 1285 | 96.6 | 1.79E−45 |
| Ceres CLONE ID no. 102088 | Arabidopsis thaliana | 1286 | 96.6 | 2.89E−45 |
| Ceres CLONE ID no. 1020238 | Arabidopsis thaliana | 1287 | 96.6 | 1.79E−45 |
| Ceres CLONE ID no. 18215 | Arabidopsis thaliana | 1288 | 94 | 4.89E−43 |
| Ceres CLONE ID no. 23214 | Arabidopsis thaliana | 1289 | 94 | 4.89E−43 |
| Ceres CLONE ID no. 111974 | Arabidopsis thaliana | 1290 | 93.8 | 2.29E−45 |
| Ceres CLONE ID no. 207629 | Arabidopsis thaliana | 1291 | 93 | 7.79E−45 |
| Ceres CLONE ID no. 3929 | Arabidopsis thaliana | 1292 | 93 | 2.70E−42 |
| Public GI no. 6979332 | Oryza sativa | 1293 | 56 | 2.29E−06 |
| Public GI no. 2437817 | Alnus glutinosa | 1294 | 51.7 | 6.10E−06 |
| Public GI no. 100409 | Petunia sp. | 1295 | 39.2 | 6.10E−06 |

TABLE 135
Percent identity to Ceres cDNA ID 23380202 (SEQ ID NO: 1297)

| | | SEQ ID | | |
|---|---|---|---|---|
| Designation | Species | NO: | % Identity | e-value |
| Public GI no. 55441974 | Brassica juncea | 1298 | 70.8 | 0 |
| Public GI no. 46399063 | Brassica napus | 1299 | 70.2 | 0 |
| Public GI no. 49182274 | Lycopersicon | 1300 | 54.7 | 0 |
| | esculentum | | | |
| Public GI no. 49182280 | Beta vulgaris | 1301 | 54.5 | 0 |
| Public GI no. 21552981 | Nicotiana tabacum | 1302 | 54.2 | 0 |
| Public GI no. 60308938 | Oryza sativa subsp. | 1303 | 49.7 | 1.49E−128 |
| | indica | | | |
| Public GI no. 34906486 | Oryza sativa subsp. | 1304 | 49.7 | 3.99E−128 |
| | japonica | | | |
| Ceres CLONE ID no. 777105 | Triticum aestivum | 1305 | 49.1 | 3.50E−120 |
| Public GI no. 33087075 | Oryza sativa subsp. | 1306 | 49 | 5.29E−126 |
| | japonica | | | |
| Ceres CLONE ID no. 404146 | Zea mays | 1307 | 48.5 | 1.19E−126 |
| Public GI no. 49182284 | Helianthus annuus | 1308 | 41.4 | 1.79E−102 |

TABLE 136
Percent identity to Ceres cDNA ID 23396143 (SEQ ID NO: 1310)

| | | SEQ | | |
|---|---|---|---|---|
| | | ID | % | |
| Designation | Species | NO: | Identity | e-value |
| Public GI no. 50948535 | Oryza sativa | 1311 | 59.9 | 5.70E−79 |
| | subsp. | | | |
| | japonica | | | |
| Public GI no. 50948537 | Oryza sativa | 1312 | 59.9 | 1.50E−72 |
| | subsp. | | | |
| | japonica | | | |
| Ceres CLONE ID no. | Glycine max | 1313 | 58.4 | 1.30E−56 |
| 476283 | | | | |
| Public GI no. 7716952 | Medicago | 1314 | 56.4 | 1.19E−57 |
| | truncatula | | | |
| Public GI no. 21105746 | Petunia x hybrida | 1315 | 53.1 | 9.00E−99 |
| Public GI no. 40647397 | Lycopersicon | 1316 | 50.3 | 9.40E−41 |
| | esculentum | | | |
| Public GI no. 34902994 | Oryza sativa | 1317 | 48.4 | 7.59E−47 |
| Public GI no. 14485513 | Solanum | 1318 | 48.3 | 2.20E−41 |
| | tuberosum | | | |
| Ceres CLONE ID no. | Zea mays | 1319 | 40.9 | 5.90E−63 |
| 461297 | | | | |

TABLE 137
Percent identity to Ceres cDNA ID 23420963 (SEQ ID NO: 1323)

| | | SEQ ID | % | |
|---|---|---|---|---|
| Designation | Species | NO: | Identity | e-value |
| Public GI | Brassica oleracea | 1324 | 96.6 | 9.39E−103 |
| no. 38196019 | | | | |
| Public GI | Sisymbrium irio | 1325 | 86.89 | 7.90E−90 |
| no. 38260618 | | | | |
| Public GI | Arabidopsis arenosa | 1326 | 84.5 | 1.90E−81 |
| no. 38260631 | | | | |
| Public GI | Arabidopsis thaliana | 1327 | 84.5 | 2.39E−81 |
| no. 9759579 | | | | |
| Public GI | Arabidopsis arenosa | 1328 | 81.28 | 3.39E−82 |
| no. 38260685 | | | | |
| Public GI | Arabidopsis pumila | 1329 | 80.79 | 3.39E−82 |
| no. 38260669 | | | | |
| Public GI | Boechera drummondii | 1330 | 80.3 | 2.50E−79 |
| no. 34013890 | | | | |
| Public GI | Capsella rubella | 1331 | 78.33 | 1.30E−80 |
| no. 38260649 | | | | |
| Public GI | Arabidopsis thaliana | 1332 | 68.5 | 1.49E−65 |
| no. 19310643 | | | | |
| Public GI | Arabidopsis thaliana | 1333 | 68 | 9.99E−65 |
| no. 21554069 | | | | |

TABLE 138
Percent identity to Ceres cDNA ID 23369680 (SEQ ID NO: 1335)

| | | SEQ ID | % | |
|---|---|---|---|---|
| Designation | Species | NO: | Identity | e-value |
| Public GI | Oryza sativa subsp. | 1336 | 62.78 | 4.09E−45 |
| no. 34902106 | japonica | | | |
| Ceres CLONE ID | Triticum aestivum | 1337 | 61.54 | 6.70E−45 |
| no. 677852 | | | | |
| Ceres CLONE ID | Glycine max | 1338 | 50 | 4.09E−38 |
| no. 637282 | | | | |

TABLE 139
Percent identity to Ceres cDNA ID 23449316 (SEQ ID NO: 1342)

| | | SEQ ID | % | |
|---|---|---|---|---|
| Designation | Species | NO: | Identity | e-value |
| Ceres CLONE | Arabidopsis thaliana | 1343 | 85 | 3.00E−40 |
| ID no. 3244 | | | | |
| Ceres CLONE | Arabidopsis thaliana | 1344 | 72.8 | 2.00E−37 |
| ID no. 23214 | | | | |
| Ceres CLONE | Arabidopsis thaliana | 1345 | 72.5 | 1.00E−36 |
| ID no. 248633 | | | | |
| Ceres CLONE | Arabidopsis thaliana | 1346 | 95 | 3.00E−44 |
| ID no. 111974 | | | | |
| Ceres CLONE | Arabidopsis thaliana | 1347 | 85.1 | 1.00E−37 |
| ID no. 20104 | | | | |
| Ceres CLONE | Arabidopsis thaliana | 1348 | 82.7 | 1.00E−35 |
| ID no. 39985 | | | | |
| Ceres CLONE | Arabidopsis thaliana | 1349 | 76.8 | 6.00E−35 |
| ID no. 207629 | | | | |

TABLE 140
Percent identity to Ceres cDNA ID 23377150 (SEQ ID NO: 1353)

| | | SEQ ID | % | |
|---|---|---|---|---|
| Designation | Species | NO: | Identity | e-value |
| Public GI | Boea crassifolia | 1354 | 73.85 | 5.60E−73 |
| no. 30575840 | | | | |
| Public GI | Populus × canescens | 1355 | 73.33 | 6.69E−77 |
| no. 22795039 | | | | |
| Ceres CLONE | Glycine max | 1356 | 70.62 | 1.29E−66 |
| ID no. 543289 | | | | |

TABLE 141
Percent identity to Ceres cDNA ID 23402435 (SEQ ID NO: 1358)

| | | SEQ | | |
|---|---|---|---|---|
| | | ID | % | |
| Designation | Species | NO: | Identity | e-value |
| Public GI | Capsicum annuum | 1359 | 68.72 | 9.20E−112 |
| no. 33320073 | | | | |
| Public GI | Arabidopsis thaliana | 1360 | 65.52 | 7.89E−106 |
| no. 15810645 | | | | |
| Ceres CLONE ID | Arabidopsis thaliana | 1361 | 65.46 | 7.20E−112 |
| no. 38311 | | | | |
| Ceres CLONE ID | Arabidopsis thaliana | 1362 | 65.46 | 9.20E−112 |
| no. 25854 | | | | |
| Public GI | Arabidopsis thaliana | 1363 | 65.46 | 1.20E−111 |
| no. 21689705 | | | | |
| Ceres CLONE ID | Arabidopsis thaliana | 1364 | 65.23 | 2.10E−105 |
| no. 19561 | | | | |
| Public GI | Arabidopsis thaliana | 1365 | 65.23 | 2.69E−105 |
| no. 21554039 | | | | |
| Public GI | Arabidopsis thaliana | 1366 | 64.72 | 3.59E−101 |
| no. 20259029 | | | | |
| Ceres CLONE ID | Arabidopsis thaliana | 1367 | 64.72 | 3.59E−101 |
| no. 1335983 | | | | |

TABLE 142
Percent identity to Ceres cDNA ID 23418435 (SEQ ID NO: 1369)

| | | SEQ ID | % | |
|---|---|---|---|---|
| Designation | Species | NO: | Identity | e-value |
| Ceres CLONE ID | Glycine max | 1370 | 69.29 | 1.09E−44 |
| no. 516050 | | | | |
| Ceres CLONE ID | Triticum aestivum | 1371 | 68.35 | 1.09E−42 |
| no. 775356 | | | | |
| Ceres CLONE ID | Glycine max | 1372 | 62.58 | 1.70E−41 |
| no. 472196 | | | | |

TABLE 143
Percent identity to Ceres cDNA ID 23367406 (SEQ ID NO: 1382)

| | | SEQ | | |
|---|---|---|---|---|
| | | ID | % | |
| Designation | Species | NO: | Identity | e-value |
| Ceres CLONE ID | Arabidopsis thaliana | 1383 | 98.48 | 0 |
| no. 142681 | | | | |
| Ceres CLONE ID | Zea mays | 1384 | 93.54 | 1.09E−129 |
| no. 1063835 | | | | |
| Ceres CLONE ID | Glycine max | 1385 | 93.16 | 6.30E−129 |
| no. 1027529 | | | | |
| Public GI | Sinapis alba | 1386 | 90.77 | 3.30E−123 |
| no. 21133 | | | | |
| Public GI | Arabidopsis thaliana | 1387 | 87.45 | 1.70E−119 |
| no. 11133887 | | | | |
| Ceres CLONE ID | Arabidopsis thaliana | 1388 | 85.16 | 1.89E−111 |
| no. 1139782 | | | | |
| Public GI | Arabidopsis thaliana | 1389 | 85.16 | 2.40E−111 |
| no. 2880056 | | | | |
| Public GI | Arabidopsis thaliana | 1390 | 83.85 | 1.20E−111 |
| no. 42569485 | | | | |
| Ceres CLONE ID | Brassica napus | 1391 | 81.37 | 2.89E−115 |
| no. 982579 | | | | |
| Public GI | Nicotiana tabacum | 1392 | 80.99 | 3.59E−110 |
| no. 7443216 | | | | |

TABLE 144
Percent identity to Ceres cDNA ID 23368554 (SEQ ID NO: 1394)

| | | SEQ ID | % | |
|---|---|---|---|---|
| Designation | Species | NO: | Identity | e-value |
| Ceres CLONE ID | Zea mays | 1395 | 56.5 | 1.20E−30 |
| no. 221673 | | | | |
| Public GI | Oryza sativa subsp. | 1396 | 56 | 1.09E−24 |
| no. 62733508 | japonica | | | |
| Ceres CLONE ID | Triticum aestivum | 1397 | 55.6 | 7.60E−31 |
| no. 633261 | | | | |
| Public GI | Oryza sativa | 1398 | 55.3 | 8.99E−28 |
| no. 14091850 | | | | |
| Ceres CLONE ID | Zea mays | 1399 | 54.7 | 6.09E−29 |
| no. 457567 | | | | |

TABLE 145
Percent identity to Ceres cDNA ID 23368864 (SEQ ID NO: 1401)

| | | SEQ ID | % | |
|---|---|---|---|---|
| Designation | Species | NO: | Identity | e-value |
| Ceres CLONE ID | Glycine max | 1402 | 52.06 | 2.20E−53 |
| no. 675752 | | | | |

TABLE 146
Percent identity to Ceres cDNA ID 23372744 (SEQ ID NO: 1404)

| | | SEQ ID | % | |
|---|---|---|---|---|
| Designation | Species | NO: | Identity | e-value |
| Public GI | Arabidopsis | 1405 | 98.4 | 9.99E−100 |
| no. 25518040 | thaliana | | | |
| Ceres CLONE ID | Brassica napus | 1406 | 91.2 | 1.20E−94 |
| no. 971321 | | | | |
| Ceres CLONE ID | Glycine max | 1407 | 76.9 | 2.70E−51 |
| no. 529941 | | | | |
| Ceres CLONE ID | Zea mays | 1408 | 68.5 | 1.09E−70 |
| no. 390400 | | | | |
| Ceres CLONE ID | Zea mays | 1409 | 67 | 1.20E−69 |
| no. 237172 | | | | |
| Ceres CLONE ID | Zea mays | 1410 | 67 | 1.20E−69 |
| no. 1403244 | | | | |
| Ceres CLONE ID | Glycine max | 1411 | 60.4 | 1.39E−63 |
| no. 516604 | | | | |

TABLE 147
Percent identity to Ceres cDNA ID 23374628 (SEQ ID NO: 1413)

| | | SEQ | | |
|---|---|---|---|---|
| | | ID | % | |
| Designation | Species | NO: | Identity | e-value |
| Public GI | Arabidopsis thaliana | 1414 | 97.6 | 5.29E−110 |
| no. 15238624 | | | | |
| Ceres CLONE ID | Zea mays | 1415 | 45.6 | 7.30E−10 |
| no. 497385 | | | | |
| Ceres CLONE ID | Triticum aestivum | 1416 | 44.7 | 4.30E−12 |
| no. 639274 | | | | |
| Public GI | Oryza sativa subsp. | 1417 | 43.1 | 5.00E−11 |
| no. 50905733 | japonica | | | |
| Ceres CLONE ID | Brassica napus | 1418 | 40.8 | 2.59E−09 |
| no. 981348 | | | | |
| Ceres CLONE ID | Glycine max | 1419 | 35.8 | 5.00E−11 |
| no. 812524 | | | | |

TABLE 148
Percent identity to Ceres cDNA ID 23516818 (SEQ ID NO: 1423)

| | | SEQ ID | % | |
|---|---|---|---|---|
| Designation | Species | NO: | Identity | e-value |
| Public GI | Petunia × hybrida | 1424 | 75.49 | 0 |
| no. 11249497 | | | | |
| Public GI | Oryza sativa subsp. | 1425 | 73.91 | 0 |
| no. 50940815 | japonica | | | |
| Public GI | Sorghum bicolor | 1426 | 73.72 | 0 |
| no. 18481718 | | | | |
| Ceres CLONE ID | Zea mays | 1427 | 73.32 | 0 |
| no. 244116 | | | | |

TABLE 149
Percent identity to Ceres cDNA ID 23699979 (SEQ ID NO: 1429)

| | | SEQ ID | % | |
|---|---|---|---|---|
| Designation | Species | NO: | Identity | e-value |
| Public GI | Arabidopsis thaliana | 1430 | 86.39 | 0 |
| no. 10177422 | | | | |
| Public GI | Arabidopsis thaliana | 1431 | 85.44 | 0 |
| no. 1764100 | | | | |
| Public GI | Arabidopsis thaliana | 1432 | 85.44 | 0 |
| no. 28373943 | | | | |
| Ceres CLONE ID | Arabidopsis thaliana | 1433 | 85.16 | 0 |
| no. 11217 | | | | |
| Public GI | Arabidopsis thaliana | 1434 | 85.16 | 0 |
| no. 21536808 | | | | |
| Public GI | Arabidopsis thaliana | 1435 | 84.89 | 0 |
| no. 6562268 | | | | |
| Public GI | Oryza sativa subsp. | 1436 | 81.94 | 0 |
| no. 55296998 | japonica | | | |
| Ceres CLONE ID | Zea mays | 1437 | 81.67 | 0 |
| no. 238929 | | | | |
| Ceres CLONE ID | Triticum aestivum | 1438 | 59.04 | 2.10E−59 |
| no. 686876 | | | | |

TABLE 150
Percent identity to Ceres cDNA ID 23814706 (SEQ ID NO: 1440)

| | | SEQ ID | % | |
|---|---|---|---|---|
| Designation | Species | NO: | Identity | e-value |
| Ceres CLONE ID | Arabidopsis thaliana | 1441 | 63.1 | 8.10E−27 |
| no. 1349 | | | | |
| Public GI | Arabidopsis thaliana | 1442 | 63.1 | 8.10E−27 |
| no. 62318582 | | | | |
| Public GI | Arabidopsis thaliana | 1443 | 61.7 | 2.70E−26 |
| no. 8778455 | | | | |
| Ceres CLONE ID | Arabidopsis thaliana | 1444 | 62.2 | 3.60E−24 |
| no. 19640 | | | | |
| Public GI | Arabidopsis thaliana | 1445 | 62.2 | 3.60E−24 |
| no. 19310623 | | | | |
| Ceres CLONE ID | Brassica napus | 1446 | 59.6 | 8.29E−25 |
| no. 1099781 | | | | |
| Ceres CLONE ID | Brassica napus | 1447 | 66.6 | 6.70E−23 |
| no. 1066463 | | | | |
| Ceres CLONE ID | Glycine max | 1448 | 61.6 | 5.69E−26 |
| no. 476445 | | | | |
| Ceres CLONE ID | Zea mays | 1449 | 93.9 | 2.79E−47 |
| no. 327449 | | | | |
| Public GI | Oryza sativa subsp. | 1450 | 85.4 | 8.80E−37 |
| no. 37991859 | japonica | | | |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of determining whether or not a regulatory region is activated by a regulatory protein comprising:
determining whether or not reporter activity is detected in a plant cell transformed with:
a) a recombinant nucleic acid construct comprising a regulatory region operably linked to a nucleic acid encoding a polypeptide having said reporter activity; and
b) a recombinant nucleic acid construct comprising a nucleic acid encoding a regulatory protein comprising a polypeptide sequence having 80% or greater sequence identity to a polypeptide sequence selected from the group consisting of SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NO:200, SEQ ID NO:205, SEQ ID NOs:211-214, SEQ ID NOs:216-223, SEQ ID NOs:225-226, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NO:632, SEQ ID NO:637, SEQ ID NO:639, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NO:703, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-907, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:1119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NO:1212, SEQ ID NOs:1220-1224, SEQ ID NOs:1226-1241, SEQ ID NOs:1243-1246, SEQ ID NO:1248, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NO:1394, SEQ ID NO:1401, SEQ ID NOs:1404-1411, SEQ ID NOs:1413-1414, SEQ ID NO:1421, SEQ ID NOs:1423-1427, SEQ ID NOs:1429-1438, SEQ ID NO:1440, SEQ ID NO:1452, SEQ ID NOs:1476-1484, and the consensus sequences set forth in FIGS. 1-140,
wherein detection of said reporter activity indicates that said regulatory region is activated by said regulatory protein.

2. The method of claim 1, wherein said activation is direct or indirect.

3. The method of claim 1, wherein said nucleic acid encoding said regulatory protein is operably linked to a regulatory region, wherein said regulatory region is capable of modulating expression of said regulatory protein.

4. The method of claim 3, wherein said regulatory region capable of modulating expression of said regulatory protein is a promoter.

5-14. (canceled)

15. The method of claim 1, wherein said reporter activity is selected from an enzymatic activity and an optical activity.

16. (canceled)

17. (canceled)

18. A method of determining whether or not a regulatory region is activated by a regulatory protein comprising:
determining whether or not reporter activity is detected in a plant cell transformed with:
a) a recombinant nucleic acid construct comprising a regulatory region comprising a nucleic acid having 80% or greater sequence identity to a regulatory region selected from the group consisting of SEQ ID NOs:1453-1468 operably linked to a nucleic acid encoding a polypeptide having said reporter activity; and
b) a recombinant nucleic acid construct comprising a nucleic acid encoding a regulatory protein;
wherein detection of said reporter activity indicates that said regulatory region is activated by said regulatory protein.

19. The method of claim 18, wherein said regulatory protein comprises a polypeptide sequence having 80% or greater sequence identity to a polypeptide sequence selected from the group consisting of SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NO:200, SEQ ID NO:205, SEQ ID NOs:211-214, SEQ ID NOs:216-223, SEQ ID NOs:225-226, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NO:632, SEQ ID NO:637, SEQ ID NO:639, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NO:703, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-907, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:1119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NO:1212, SEQ ID NOs:1220-1224, SEQ ID NOs:1226-1241, SEQ ID NOs:1243-1246, SEQ ID NO:1248, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NO:1394, SEQ ID NO:1401, SEQ ID NOs:1404-1411, SEQ ID NOs:1413-1414, SEQ ID NO:1421, SEQ ID NOs:1423-1427, SEQ ID NOs:1429-1438, SEQ ID NO:1440, SEQ ID NO:1452, SEQ ID NOs:1476-1484, and the consensus sequences set forth in FIGS. 1-140.

20. A plant cell comprising an exogenous nucleic acid, said exogenous nucleic acid comprising a nucleic acid encoding a regulatory protein comprising a polypeptide sequence having 80% or greater sequence identity to a polypeptide sequence selected from the group consisting of SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NO:200, SEQ ID NO:205, SEQ ID NOs:211-214, SEQ ID NOs:216-223, SEQ ID NOs:225-226, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NO:632, SEQ ID NO:637, SEQ ID NO:639, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NO:703, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-907, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:1119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NO:1212, SEQ ID NOs:1220-1224, SEQ ID NOs:1226-1241, SEQ ID NOs:1243-1246, SEQ ID NO:1248, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NO:1394, SEQ ID NO:1401, SEQ ID NOs:1404-1411, SEQ ID NOs:1413-1414, SEQ ID NO:1421, SEQ ID NOs:1423-1427, SEQ ID NOs:1429-1438, SEQ ID NO:1440, SEQ ID NO:1452, SEQ ID NOs:1476-1484, and the consensus sequences set forth in FIGS. 1-140, wherein said nucleic acid is operably linked to a regulatory region that modulates transcription of said regulatory protein in said plant cell.

21. The plant cell of claim 20, wherein said regulatory region is a promoter.

22-28. (canceled)

29. The plant cell of claim 20, wherein said plant cell is capable of producing one or more alkaloids.

30. The plant cell of claim 20, wherein said plant cell further comprises an endogenous regulatory region that is associated with said regulatory protein.

31. The plant cell of claim 20, wherein said regulatory protein modulates transcription of an endogenous gene involved in alkaloid biosynthesis in said cell.

32. The plant cell of claim 31, wherein said endogenous gene comprises a coding sequence for an alkaloid biosynthesis enzyme.

33. The plant cell of claim 31, wherein said endogenous gene comprises a coding sequence for a regulatory protein involved in alkaloid biosynthesis.

34. (canceled)

35. The plant cell of claim 32, wherein said endogenous gene is a tetrahydrobenzylisoquinoline alkaloid biosynthesis enzyme, a benzophenanthridine alkaloid biosynthesis enzyme, a morphinan alkaloid biosynthesis enzyme, a monoterpenoid indole alkaloid biosynthesis enzyme, a bisbenzylisoquinoline alkaloid biosynthesis enzyme, a pyridine, purine, tropane, or quinoline alkaloid biosynthesis enzyme, a terpenoid, betaine, or phenethylamine alkaloid biosynthesis enzyme, or a steroid alkaloid biosynthesis enzyme.

36. The plant cell of claim 31, wherein said endogenous gene is selected from the group consisting of tyrosine decarboxylase (YDC or TYD; EC 4.1.1.25), norcoclaurine synthase (EC 4.2.1.78), coclaurine N-methyltransferase (EC 2.1.1.140), (R,S)-norcoclaurine 6-O-methyl transferase (NOMT; EC 2.1.1.128), S-adenosyl-L-methionine:3′-hydroxy-N-methylcoclaurine 4′-O-methyltransferase 1 (HMCOMT1; EC 2.1.1.116); S-adenosyl-L-methionine:3′-hydroxy-N-methylcoclaurine 4′-O-methyltransferase 2 (HMCOMT2; EC 2.1.1.116); monophenol monooxygenase (EC 1.14.18.1), N-methylcoclaurine 3′-hydroxylase (NMCH; EC 1.14.13.71), (R,S)-reticuline 7-O-methyltransferase (ROMT); berbamunine synthase (EC 1.14.21.3), columbamine O-methyltransferase (EC 2.1.1.118), berberine bridge enzyme (BBE; (EC 1.21.3.3), reticuline oxidase (EC 1.21.3.4), dehydro reticulinium ion reductase (EC 1.5.1.27), (RS)-1-benzyl-1,2,3,4-tetrahydroisoquinoline N-methyltransferase (EC 2.1.1.115), (S)-scoulerine oxidase (EC 1.14.21.2), (S)-cheilanthifoline oxidase (EC 1.14.21.1), (S)-tetrahydroprotoberberine N-methyltransferase (EC 2.1.1.122), (S)-canadine synthase (EC 1.14.21.5), tetrahydroberberine oxidase (EC 1.3.3.8), and columbamine oxidase (EC 1.21.3.2).

37. The plant cell of claim 31, wherein said endogenous gene is selected from the group consisting of those encoding for dihydrobenzophenanthridine oxidase (EC 1.5.3.12), dihydrosanguinarine 10-hydroxylase (EC 1.14.13.56), 10-hydroxydihydrosanguinarine 10-O-methyltransferase (EC 2.1.1.119), dihydrochelirubine 12-hydroxylase (EC 1.14.13.57), and 12-hydroxydihydrochelirubine 12-O-methyltransferase (EC 2.1.1.120).

38. The plant cell of claim 31, wherein said endogenous gene is selected from the group consisting of those encoding for salutaridinol 7-O-acetyltransferase (SAT; EC 2.3.1.150), salutaridine synthase (EC 1.14.21.4), salutaridine reductase (EC 1.1.1.248), morphine 6-dehydrogenase (EC 1.1.1.218); and codeinone reductase (CR; EC 1.1.1.247).

39. The plant cell of claim 20, wherein said plant cell further comprises an exogenous regulatory region operably linked to a sequence of interest, wherein said exogenous regulatory region is associated with said regulatory protein, and wherein said exogenous regulatory region comprises a nucleic acid having 80% or greater sequence identity to a regulatory region selected from the group consisting of SEQ ID NOs:1453-1468.

40. The plant cell of claim 30 wherein said plant cell is capable of producing one or more alkaloids.

41. The plant cell of claim 40, wherein at least one of said one or more alkaloids is a morphinan alkaloid or a morphinan analog alkaloid.

42. The plant cell of claim 40, wherein at least one of said one or more alkaloid compounds is a tetrahydrobenzylisoquinoline alkaloid.

43. The plant cell of claim 40, wherein at least one of said one or more alkaloids is a benzophenanthridine alkaloid.

44. The plant cell of claim 40, wherein at least one of said one or more alkaloids is a monoterpenoid indole alkaloid, a bisbenzylisoquinoline alkaloid, a pyridine, purine, tropane, or quinoline alkaloid, a terpenoid, betaine, or phenethylamine alkaloid, or a steroid alkaloid.

45. The plant cell of claim 40, wherein said plant is a member of the Papaveraceae, Menispermaceae, Lauraceae, Euphorbiaceae, Berberidaceae, Leguminosae, Boraginaceae, Apocynaceae, Asclepiadaceae, Liliaceae, Gnetaceae, Erythroxylaceae, Convolvulaceae, Ranunculaeceae, Rubiaceae, Solanaceae, or Rutaceae families.

46-48. (canceled)

49. The plant cell of claim 39, wherein said sequence of interest comprises a coding sequence for a polypeptide involved in alkaloid biosynthesis.

50. The plant cell of claim 49, wherein said polypeptide is an alkaloid biosynthesis enzyme.

51. The plant cell of claim 49, wherein said polypeptide is a regulatory protein involved in alkaloid biosynthesis.

52. The plant cell of claim 50, wherein said enzyme is a morphinan alkaloid biosynthesis enzyme.

53. The plant cell of claim 50, wherein said enzyme is a tetrahydrobenzylisoquinoline alkaloid biosynthesis enzyme.

54. The plant cell of claim 50, wherein said enzyme is a benzophenanthridine alkaloid biosynthesis enzyme.

55. The plant cell of claim 50, wherein said enzyme is a monoterpenoid indole alkaloid biosynthesis enzyme, a bisbenzylisoquinoline alkaloid biosynthesis enzyme, a pyridine, purine, tropane, or quinoline alkaloid biosynthesis enzyme, a terpenoid, betaine, or phenethylamine alkaloid biosynthesis enzyme, or a steroid alkaloid biosynthesis enzyme.

56. The plant cell of claim 50, wherein said enzyme is selected from the group consisting of salutaridinol 7-O-acetyltransferase (SAT; EC 2.3.1.150), salutaridine synthase (EC 1.14.21.4), salutaridine reductase (EC 1.1.1.248), morphine 6-dehydrogenase (EC 1.1.1.218); and codeinone reductase (CR; EC 1.1.1.247).

57. The plant cell of claim 50, wherein said enzyme is selected from the group consisting of tyrosine decarboxylase (YDC or TYD; EC 4.1.1.25), norcoclaurine synthase (EC 4.2.1.78), coclaurine N-methyltransferase (EC 2.1.1.140), (R,S)-norcoclaurine 6-O-methyl transferase (NOMT; EC 2.1.1.128), S-adenosyl-L-methionine:3′-hydroxy-N-methylcoclaurine 4′-O-methyltransferase 1 (HMCOMT1; EC 2.1.1.116); S-adenosyl-L-methionine:3′-hydroxy-N-methylcoclaurine 4′-O-methyltransferase 2 (HMCOMT2; EC 2.1.1.116); monophenol monooxygenase (EC 1.14.18.1), N-methylcoclaurine 3′-hydroxylase (NMCH; EC 1.14.13.71), (R,S)-reticuline 7-O-methyltransferase (ROMT); berbamunine synthase (EC 1.14.21.3), columbamine O-methyltransferase (EC 2.1.1.118), berberine bridge enzyme (BBE; (EC 1.21.3.3), reticuline oxidase (EC 1.21.3.4), dehydro reticulinium ion reductase (EC 1.5.1.27), (RS)-1-benzyl-1,2,3,4-tetrahydroisoquinoline N-methyltransferase (EC 2.1.1.115), (S)-scoulerine oxidase (EC 1.14.21.2), (S)-cheilanthifoline oxidase (EC 1.14.21.1), (S)-tetrahydroprotoberberine N-methyltransferase (EC 2.1.1.122), (S)-canadine synthase (EC 1.14.21.5), tetrahydroberberine oxidase (EC 1.3.3.8), and columbamine oxidase (EC 1.21.3.2).

58. The plant cell of claim 50, wherein said enzyme is selected from the group consisting of dihydrobenzophenanthridine oxidase (EC 1.5.3.12), dihydrosanguinarine 10-hydroxylase (EC 1.14.13.56), 10-hydroxydihydrosanguinarine 10-O-methyltransferase (EC 2.1.1.119), dihydrochelirubine 12-hydroxylase (EC 1.14.13.57), and 12-hydroxydihydrochelirubine 12-O-methyltransferase (EC 2.1.1.120).

59. The plant cell of claim 30 wherein said regulatory protein-regulatory region association is effective for modulating the amount of at least one alkaloid compound in said cell.

60. The plant cell of claim 59, wherein said at least one alkaloid compound is selected from the group consisting of salutaridine, salutaridinol, salutaridinol acetate, thebaine, isothebaine, papaverine, narcotine, noscapine, narceine, hydrastine, oripavine, morphinone, morphine, codeine, codeinone, and neopinone.

61. The plant cell of claim 59, wherein said at least one alkaloid compound is selected from the group consisting of berberine, palmatine, tetrahydropalmatine, S-canadine, columbamine, S-tetrahydrocolumbamine, S-scoulerine, S-cheilathifoline, S-stylopine, S-cis-N-methylstylopine, protopine, 6-hydroxyprotopine, R-norreticuline, S-norreticuline, R-reticuline, S-reticuline, 1,2-dehydroreticuline, S-3′-hydroxycoclaurine, S-norcoclaurine, S-coclaurine, S—N-methylcoclaurine, berbamunine, 2′-norberbamunine, and guatteguamerine.

62. The plant cell of claim 59, wherein said at least one alkaloid compound is selected from the group consisting of dihydro-sanguinarine, sanguinarine, dihydroxy-dihydro-sanguinarine, 12-hydroxy-dihydrochelirubine, 10-hydroxy-dihydro-sanguinarine, dihydro-macarpine, dihydro-chelirubine, dihydro-sanguinarine, chelirubine, 12-hydroxy-chelirubine, and macarpine.

63. A Papaveraceae plant comprising an exogenous nucleic acid, said exogenous nucleic acid comprising a nucleic acid encoding a regulatory protein comprising a polypeptide sequence having 80% or greater sequence identity to a polypeptide sequence selected from the group consisting of SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NO:200, SEQ ID NO:205, SEQ ID NOs:211-214, SEQ ID NOs:216-223, SEQ ID NOs:225-226, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NO:632, SEQ ID NO:637, SEQ ID NO:639, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NO:703, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-907, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:1119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NO:1212, SEQ ID NOs:1220-1224, SEQ ID NOs:1226-1241, SEQ ID NOs:1243-1246, SEQ ID NO:1248, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NO:1394, SEQ ID NO:1401, SEQ ID NOs:1404-1411, SEQ ID NOs:1413-1414, SEQ ID NO:1421, SEQ ID NOs:1423-1427, SEQ ID NOs:1429-1438, SEQ ID NO:1440, SEQ ID NO:1452, SEQ ID NOs:1476-1484, and the consensus sequences set forth in FIGS. 1-140, wherein said nucleic acid is operably linked to a regulatory region that modulates transcription of said regulatory protein in said plant cell.

64. A method of expressing a sequence of interest comprising:
growing a plant cell comprising:
a) an exogenous nucleic acid comprising a regulatory region comprising a nucleic acid having 80% or greater sequence identity to a regulatory region selected from the group consisting of SEQ ID NOs:1453-1468, wherein said regulatory region is operably linked to a sequence of interest; and
b) an exogenous nucleic acid comprising a nucleic acid encoding a regulatory protein comprising a polypeptide sequence having 80% or greater sequence identity to a polypeptide sequence selected from the group consisting of SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NO:200, SEQ ID NO:205, SEQ ID NOs:211-214, SEQ ID NOs:216-223, SEQ ID NOs:225-226, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NO:632, SEQ ID NO:637, SEQ ID NO:639, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NO:703, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-907, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:1119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NO:1212, SEQ ID NOs:1220-1224, SEQ ID NOs:1226-1241, SEQ ID NOs:1243-1246, SEQ ID NO:1248, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NO:1394, SEQ ID NO:1401, SEQ ID NOs:1404-1411, SEQ ID NOs:1413-1414, SEQ ID NO:1421, SEQ ID NOs:1423-1427, SEQ ID NOs:1429-1438, SEQ ID NO:1440, SEQ ID NO:1452, SEQ ID NOs:1476-1484, and the consensus sequences set forth in FIGS. 1-140;
wherein said regulatory region and said regulatory protein are associated, and wherein said plant cell is grown under conditions effective for the expression of said regulatory protein.

65. A method of expressing an endogenous sequence of interest comprising growing a plant cell comprising an endogenous regulatory region operably linked to a sequence of interest, wherein said endogenous regulatory region comprises a nucleic acid having 80% or greater sequence identity to a regulatory region selected from the group consisting of SEQ ID NOs:1453-1468, wherein said plant cell further comprises a nucleic acid encoding an exogenous regulatory protein, said exogenous regulatory protein comprising a polypeptide sequence having 80% or greater sequence identity to a polypeptide sequence selected from the group consisting of SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NO:200, SEQ ID NO:205, SEQ ID NOs:211-214, SEQ ID NOs:216-223, SEQ ID NOs:225-226, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NO:632, SEQ ID NO:637, SEQ ID NO:639, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NO:703, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-907, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:1119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NO:1212, SEQ ID NOs:1220-1224, SEQ ID NOs:1226-1241, SEQ ID NOs:1243-1246, SEQ ID NO:1248, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NO:1394, SEQ ID NO:1401, SEQ ID NOs:1404-1411, SEQ ID NOs:1413-1414, SEQ ID NO:1421, SEQ ID NOs:1423-1427, SEQ ID NOs:1429-1438, SEQ ID NO:1440, SEQ ID NO:1452, SEQ ID NOs:1476-1484, and the consensus sequences set forth in FIGS. 1-140, wherein said exogenous regulatory protein and said endogenous regulatory region are associated, wherein said plant cell is grown under conditions effective for the expression of said exogenous regulatory protein.

66. A method of expressing an exogenous sequence of interest comprising growing a plant cell comprising an exogenous regulatory region operably linked to a sequence of interest, wherein said exogenous regulatory region comprises a nucleic acid having 80% or greater sequence identity to a regulatory region selected from the group consisting of SEQ ID NOs:1453-1468, wherein said plant cell further comprises a nucleic acid encoding an endogenous regulatory protein, said endogenous regulatory protein comprising a polypeptide sequence having 80% or greater sequence identity to a polypeptide sequence selected from the group consisting of SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NO:200, SEQ ID NO:205, SEQ ID NOs:211-214, SEQ ID NOs:216-223, SEQ ID NOs:225-226, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NO:632, SEQ ID NO:637, SEQ ID NO:639, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NO:703, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-907, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:1119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NO:1212, SEQ ID NOs:1220-1224, SEQ ID NOs:1226-1241, SEQ ID NOs:1243-1246, SEQ ID NO:1248, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NO:1394, SEQ ID NO:1401, SEQ ID NOs:1404-1411, SEQ ID NOs:1413-1414, SEQ ID NO:1421, SEQ ID NOs:1423-1427, SEQ ID NOs:1429-1438, SEQ ID NO:1440, SEQ ID NO:1452, SEQ ID NOs:1476-1484, and the consensus sequences set forth in FIGS. 1-140, wherein said regulatory region and said regulatory protein are associated, and wherein said plant cell is grown under conditions effective for the expression of said endogenous regulatory protein.

67. The method of claim 65, wherein said sequence of interest comprises a coding sequence for a polypeptide involved in alkaloid biosynthesis.

68-71. (canceled)

72. A method of modulating the expression level of one or more endogenous Papaveraceae genes involved in alkaloid biosynthesis, said method comprising transforming a cell of a member of the Papaveraceae family with a recombinant nucleic acid construct, wherein said nucleic acid construct comprises a nucleic acid encoding a regulatory protein comprising a polypeptide sequence selected from the group consisting of SEQ ID NOs:80-84, SEQ ID NOs:86-91, SEQ ID NO:93, SEQ ID NOs:95-111, SEQ ID NO:113, SEQ ID NOs:115-119, SEQ ID NO:121, SEQ ID NOs:123-139, SEQ ID NOs:141-142, SEQ ID NOs:144-150, SEQ ID NOs:152-156, SEQ ID NOs:158-166, SEQ ID NOs:168-171, SEQ ID NOs:173-185, SEQ ID NOs:187-198, SEQ ID NO:200, SEQ ID NO:205, SEQ ID NOs:211-214, SEQ ID NOs:216-223, SEQ ID NOs:225-226, SEQ ID NOs:229-233, SEQ ID NOs:235-244, SEQ ID NOs:246-258, SEQ ID NOs:260-262, SEQ ID NOs:264-279, SEQ ID NOs:281-286, SEQ ID NOs:288-299, SEQ ID NOs:301-307, SEQ ID NOs:309-323, SEQ ID NOs:325-331, SEQ ID NOs:333-343, SEQ ID NOs:345-348, SEQ ID NOs:350-354, SEQ ID NOs:356-362, SEQ ID NOs:364-366, SEQ ID NO:368, SEQ ID NOs:370-374, SEQ ID NOs:376-380, SEQ ID NOs:382-385, SEQ ID NOs:387-390, SEQ ID NOs:392-399, SEQ ID NOs:401-409, SEQ ID NOs:411-417, SEQ ID NOs:419-432, SEQ ID NOs:434-448, SEQ ID NOs:450-456, SEQ ID NOs:458-464, SEQ ID NOs:466-470, SEQ ID NOs:472-488, SEQ ID NO:490, SEQ ID NO:492, SEQ ID NOs:494-504, SEQ ID NOs:506-514, SEQ ID NOs:516-521, SEQ ID NOs:523-530, SEQ ID NOs:532-546, SEQ ID NOs:548-561, SEQ ID NO:563, SEQ ID NOs:565-568, SEQ ID NO:570, SEQ ID NO:572, SEQ ID NOs:574-577, SEQ ID NOs:579-588, SEQ ID NOs:590-591, SEQ ID NOs:593-597, SEQ ID NOs:599-606, SEQ ID NOs:608-611, SEQ ID NOs:613-617, SEQ ID NOs:619-630, SEQ ID NO:632, SEQ ID NO:637, SEQ ID NO:639, SEQ ID NOs:648-650, SEQ ID NOs:652-655, SEQ ID NO:657, SEQ ID NOs:659-662, SEQ ID NOs:664-669, SEQ ID NOs:671-672, SEQ ID NOs:674-677, SEQ ID NOs:679-684, SEQ ID NOs:686-693, SEQ ID NOs:695-696, SEQ ID NOs:698-699, SEQ ID NO:701, SEQ ID NO:703, SEQ ID NOs:711-714, SEQ ID NOs:716-719, SEQ ID NOs:721-730, SEQ ID NOs:732-746, SEQ ID NOs:748-758, SEQ ID NOs:760-764, SEQ ID NOs:766-767, SEQ ID NOs:769-775, SEQ ID NOs:777-790, SEQ ID NOs:792-795, SEQ ID NOs:797-810, SEQ ID NOs:812-818, SEQ ID NO:820, SEQ ID NOs:822-826, SEQ ID NOs:828-832, SEQ ID NOs:834-838, SEQ ID NOs:840-843, SEQ ID NOs:845-849, SEQ ID NOs:851-854, SEQ ID NOs:856-867, SEQ ID NO:869, SEQ ID NOs:871-872, SEQ ID NOs:874-887, SEQ ID NOs:889-904, SEQ ID NOs:906-907, SEQ ID NOs:921-929, SEQ ID NOs:931-944, SEQ ID NOs:946-962, SEQ ID NOs:964-971, SEQ ID NOs:973-981, SEQ ID NOs:983-990, SEQ ID NOs:992-999, SEQ ID NOs:1001-1017, SEQ ID NOs:1019-1024, SEQ ID NOs:1026-1040, SEQ ID NOs:1042-1056, SEQ ID NOs:1058-1066, SEQ ID NOs:1068-1072, SEQ ID NOs:1074-1085, SEQ ID NOs:1087-1100, SEQ ID NOs:1102-1117, SEQ ID NOs:1119-1125, SEQ ID NOs:1127-1136, SEQ ID NOs:1138-1145, SEQ ID NOs:1147-1156, SEQ ID NOs:1158-1163, SEQ ID NOs:1165-1169, SEQ ID NOs:1171-1176, SEQ ID NOs:1178-1190, SEQ ID NOs:1192-1200, SEQ ID NOs:1202-1208, SEQ ID NO:1210, SEQ ID NO:1212, SEQ ID NOs:1220-1224, SEQ ID NOs:1226-1241, SEQ ID NOs:1243-1246, SEQ ID NO:1248, SEQ ID NOs:1255-1259, SEQ ID NOs:1261-1277, SEQ ID NOs:1279-1295, SEQ ID NOs:1297-1308, SEQ ID NOs:1310-1319, SEQ ID NO:1321, SEQ ID NOs:1323-1333, SEQ ID NOs:1335-1338, SEQ ID NO:1340, SEQ ID NOs:1342-1349, SEQ ID NO:1351, SEQ ID NOs:1353-1356, SEQ ID NOs:1358-1367, SEQ ID NOs:1369-1372, SEQ ID NO:1374, SEQ ID NO:1376, SEQ ID NO:1378, SEQ ID NO:1380, SEQ ID NOs:1382-1392, SEQ ID NO:1394, SEQ ID NO:1401, SEQ ID NOs:1404-1411, SEQ ID NOs:1413-1414, SEQ ID NO:1421, SEQ ID NOs:1423-1427, SEQ ID NOs:1429-1438, SEQ ID NO:1440, SEQ ID NO:1452, SEQ ID NOs:1476-1484, and the consensus sequences set forth in FIGS. 1-140, and wherein said nucleic acid is operably linked to a regulatory region that modulates transcription in the family member.

73-75. (canceled)
